(19) United States
(12) Patent Application Publication
(10) Pub. No.: US 2019/0351040 A1
(43) Pub. Date: Nov. 21, 2019

(54) RNA CANCER VACCINES

(71) Applicant: ModernaTX, Inc., Cambridge, MA (US)

(72) Inventors: Nicholas Valiante, Cambridge, MA (US); Ted Ashburn, Boston, MA (US); Kristen Flopson, Arlington, MA (US)

(73) Assignee: ModernaTX, Inc., Cambridge, MA (US)

(21) Appl. No.: 16/482,844

(22) PCT Filed: Oct. 26, 2017
(86) PCT No.: PCT/US2017/058595

§ 371 Date: Aug. 1, 2019

Related U.S. Application Data
(60) Provisional application No. 62/558,238, filed on Sep. 13, 2017.
(60) Provisional application No. 62/453,444, filed on Feb. 1, 2017.
(60) Provisional application No. 62/453,465, filed on Feb. 1, 2017.

(51) Int. Cl. A61K 39/00; A61K 39/39

(52) U.S. Cl. CPC A61K 39/001164; A61K 39/39; A61K 2039/55555; A61K 2039/505; A61K 2039/55516; A61K 2039/53

(57) ABSTRACT

The disclosure relates to cancer ribonucleic acid (RNA) vaccines, as well as methods of using the vaccines and compositions comprising the vaccines. In particular, the disclosure relates to concatemeric mRNA cancer vaccines encoding several cancer epitopes on a single mRNA construct, i.e. poly-epitope mRNA constructs or poly-neo-epitope constructs. The disclosure further relates to p53 and KRAS mutations, as well as incorporation of immune enhancers such as STING, e.g. mRNA constructs further encoding an immune stimulator or adjuvant. The disclosure further relates to inclusion of universal T cell epitopes, such as tetanus or diphtheria toxins to elicit an enhanced immune response.

### RELATED APPLICATIONS

This application claims the benefit under 35 U.S.C. 119(e) of the filing date of U.S. Provisional Application Ser. No. 62/453,444, filed Feb. 1, 2017, entitled “RNA CANCER VACCINES”, of U.S. Provisional Application Ser. No. 62/453,465, filed Feb. 1, 2017, entitled “IMMUNOMODULATORY THERAPEUTIC MRNA COMPOSITIONS ENCODING ACTIVATING ONCOGENE MUTATION PEPTIDES”, and of U.S. Provisional Application Ser. No. 62/558,238, filed Sep. 13, 2017, entitled “CONCATAMERIC RNA CANCER VACCINES”, the entire contents of each of which are incorporated herein by reference.

### BACKGROUND OF INVENTION

Recent theories in cancer evolution have focused on three steps including stress-induced genome instability, population diversity or heterogeneity, and genome-mediated macroevolution. The theory explains why most of the known molecular mechanisms can contribute to cancer yet there is no single dominant mechanism for the majority of clinical cases. However, the common mechanisms suggest that cancer vaccines may provide a universal solution in the treatment of cancer.

Cancer vaccines include preventive or prophylactic vaccines, which are intended to prevent cancer from developing in healthy people; and therapeutic vaccines, which are intended to treat an existing cancer by strengthening the body's natural defenses against the cancer. Cancer preventive vaccines may, for instance, target infectious agents that cause or contribute to the development of cancer in order to prevent infectious diseases from causing cancer. Gardasil® and Cervarix®, are two examples of commercially available prophylactic vaccines. Each vaccine protects against HPV infection. Other preventive cancer vaccines may target host proteins or fragments that are predicted to increase the likelihood of an individual developing cancer in the future.

Most commercial or developing vaccines (e.g., cancer vaccines) are based on whole microorganisms, protein antigens, peptides, polysaccharides or deoxyribonucleic acid (DNA) vaccines and their combinations. DNA vaccination is one technique used to stimulate humoral and cellular immune responses to antigens. The direct injection of genetically engineered DNA (e.g., naked plasmid DNA) into a living host results in a small number of its cells directly producing an antigen, resulting in a protective immunological response. With this technique, however, comes potential problems of DNA integration into the vaccine's genome, including the possibility of insertional mutagenesis, which could lead to the activation of oncogenes or the inhibition of tumor suppressor genes.

### SUMMARY OF INVENTION

Provided herein is a ribonucleic acid (RNA) cancer vaccine of an RNA (e.g., messenger RNA (mRNA)) that can safely direct the body's cellular machinery to produce nearly any cancer protein or fragment thereof of interest. In some embodiments, the RNA is a modified RNA. The RNA vaccines of the present disclosure may be used to induce a balanced immune response against cancers, comprising both cellular and humoral immunity, without risking the possibility of insertional mutagenesis, for example.

The RNA vaccines may be utilized in various settings depending on the prevalence of the cancer or the degree or level of unmet medical need. The RNA vaccines may be utilized to treat and/or prevent a cancer of various stages or degrees of metastasis. The RNA vaccines have superior properties in that they produce much larger antibody titers and produce responses earlier than alternative anti-cancer therapies including cancer vaccines. While not wishing to be bound by theory, it is believed that the RNA vaccines, as mRNA polynucleotides, are better designed to produce the appropriate protein conformation upon translation as the RNA vaccines co-opt natural cellular machinery. Unlike traditional therapies and vaccines which are manufactured ex vivo and may trigger unwanted cellular responses, the RNA vaccines are presented to the cellular system in a more native fashion.

The RNA vaccines may include a ribonucleic acid (RNA) polynucleotide having an open reading frame encoding at least one cancer antigenic polypeptide or an immunogenic fragment thereof (e.g., an immunogenic fragment capable of inducing an immune response to cancer). Other embodiments include at least one ribonucleic acid (RNA) polynucleotide having an open reading frame encoding two or more antigens or epitopes capable of inducing an immune response to cancer.

The invention in some aspects is an mRNA cancer vaccine of one or more mRNA each having an open reading frame encoding a cancer antigen peptide epitope formulated in a lipid nanoparticle, wherein the mRNA vaccine encodes 5-100 peptide epitopes and at least two of the peptide epitopes are personalized cancer antigens, and a pharmaceutically acceptable carrier or excipient.

The disclosure, in some aspects, provides an mRNA cancer vaccine comprising a lipid nanoparticle comprising one or more mRNA each having one or more open reading frames encoding 1-500 peptide epitopes which are personalized cancer antigens and a universal type II T-cell epitope.

The disclosure, in some aspects, provides an mRNA cancer vaccine comprising a lipid nanoparticle comprising one or more of the following: (a) one or more mRNA each having one or more open reading frames encoding 1-500 peptide epitopes which are personalized cancer antigens and a universal type II T-cell epitope; (b) one or more mRNA each having an open reading frame encoding an activating oncogene mutation peptide, optionally wherein the mRNA further comprises a universal type II T-cell epitope; (c) one or more mRNA each having an open reading frame encoding a cancer antigen peptide epitope, wherein the mRNA vaccine encodes 5-100 peptide epitopes and at least two of the peptide epitopes are personalized cancer antigens, optionally wherein the mRNA further comprises a universal type II T-cell epitope; and/or (d) one or more mRNA each having an open reading frame encoding a cancer antigen peptide epitope, wherein the mRNA vaccine encodes 5-100 peptide epitopes and at least three of the peptide epitopes are complex variants and at least two of the peptide epitopes are point mutations, optionally wherein the mRNA further comprises a universal type II T-cell epitope. In some embodiments, the mRNA cancer vaccine encodes 1-20 universal type II T-cell epitopes. In other embodiments, the universal type II T-cell epitope is selected from the group consisting of: ILMQYIKANSKFIGI (Tetanus toxin; SEQ ID NO: 226), FNNFTVSFWLRVPKVSASHLE, (Tetanus toxin; SEQ ID NO: 227), QYIKANSKFIGITE (Tetanus toxin; SEQ ID NO: 228) QSIALSSLMVAQAIP (Diptheria toxin; SEQ ID NO: 229), and AKFVAAWTLKAAA (pan-DR epitope; SEQ ID NO: 230).

In some embodiments, the universal type II T-cell epitope is the same universal type II T-cell epitope throughout the mRNA. In other embodiments, the universal type II T-cell epitope is repeated 1-20 times in the mRNA. In one embodiment, the universal type II T-cell epitopes are different from one another throughout the mRNA. In some embodiments, the universal type II T-cell epitope is located between every cancer antigen peptide epitope. In another embodiment, the universal type II T-cell epitope is located between every other cancer antigen peptide epitope. In one embodiment, the universal type II T-cell epitope is located between every third cancer antigen peptide epitope.

In some embodiments, one or more of the following conditions are met: (i) the activating oncogene mutation is a KRAS mutation; (ii) the KRAS mutation is a G12 mutation, optionally wherein the G12 KRAS mutation is selected from a G12D, G12V, G12S, G12C, G12A, and a G12R KRAS mutation; (iii) the KRAS mutation is a G13 mutation, optionally wherein the G13 KRAS mutation is a G13D KRAS mutation; and/or (iv) the activating oncogene mutation is a H-RAS or N-RAS mutation.

In some embodiments, one or more of the following conditions are met: (A) the mRNA has an open reading frame encoding a concatemer of two or more activating oncogene mutation peptides; (B) at least two of the peptide epitopes are separated from one another by a single Glycine, optionally wherein all of the peptide epitopes are separated from one another by a single Glycine; (C) the concatemer comprises 3-10 activating oncogene mutation peptides; and/or (D) at least two of the peptide epitopes are linked directly to one another without a linker.

In certain embodiments, one or more of the following conditions are met: (i) at least one of the peptide epitopes is a traditional cancer antigen; (ii) at least one of the peptide epitopes is a recurrent polymorphism; (iii) the recurrent polymorphism comprises a recurrent somatic cancer mutation in p53; (iv) the recurrent somatic cancer mutation in p53 is selected from the group consisting of: (A) mutations at the canonical 5′ splice site neighboring codon p.T125, inducing a retained intron having peptide sequence TAKSVTCTVSCPEGLASMRLQCLAVSPCISFVWNFGIPLHPLASCQCFFIVYPLNV (SEQ ID NO: 232) that contains epitopes AVSPCISFVW (SEQ ID NO: 233) (HLA-B*57:01, HLA-B*58:01), HPLASCQCFF (SEQ ID NO: 234) (HLA-B*35:01, HLA-B*53:01), FVWNFGIPL (SEQ ID NO: 235) (HLA-A*02:01, HLA-A*02:06, HLA-B*35:01); (B) mutations at the canonical 5′ splice site neighboring codon p.331, inducing a retained intron having peptide sequence EYFTLQVLSLGTSYQVESFQSNTQNAVFFLTVLPAIGAFAIRGQ (SEQ ID NO: 236) that contains epitopes LQVLSLGTSY (SEQ ID NO: 237) (HLA-B*15:01), FQSNTQNAVF (SEQ ID NO: 238) (HLA-B*15:01); (C) mutations at the canonical 3′ splice site neighboring codon p.126, inducing a cryptic alternative exonic 3′ splice site producing the novel spanning peptide sequence AKSVTCTMFCQLAK (SEQ ID NO: 239) that contains epitopes CTMFCQLAK (SEQ ID NO: 240) (HLA-A*11:01), KSVTCTMF (SEQ ID NO: 241) (HLA-B*58:01); and/or (D) mutations at the canonical 5′ splice site neighboring codon p.224, inducing a cryptic alternative intronic 5′ splice site producing the novel spanning peptide sequence VPYEPPEVWLALTVPPSTAWAA (SEQ ID NO: 242) that contains epitopes VPYEPPEVW (SEQ ID NO: 243) (HLA-B*53:01, HLA-B*51:01), LTVPPSTAW (SEQ ID NO: 244) (HLA-B*58:01, HLA-B*57:01), wherein the transcript codon positions refer to the canonical full-length p53 transcript ENST00000269305 (SEQ ID NO: 245) from the Ensembl v83 human genome annotation; and/or (v) the mRNA cancer vaccine does not comprise a stabilizing agent.

In some embodiments, the one or more mRNA further comprise an open reading frame encoding an immune potentiator. In other embodiments, the immune potentiator is formulated in the lipid nanoparticle. In one embodiment, the immune potentiator is formulated in a separate lipid nanoparticle. In some embodiments, the immune potentiator is a constitutively active human STING polypeptide. In one embodiment, the constitutively active human STING polypeptide comprises the amino acid sequence shown in SEQ ID NO: 1. In another embodiment, the mRNA encoding the constitutively active human STING polypeptide comprises the nucleotide sequence shown in SEQ ID NO: 170. In some embodiments, the mRNA encoding the constitutively active human STING polypeptide comprises a 3′ UTR having a miR-122 microRNA binding site. In one embodiment, the miR-122 microRNA binding site comprises the nucleotide sequence shown in SEQ ID NO: 175.

In some embodiments, the one or more mRNA each comprise a 5′ UTR comprising the nucleotide sequence set forth in SEQ ID NO: 176. In one embodiment, the one or more mRNA each comprise a poly A tail. In one embodiment, the poly A tail comprises about 100 nucleotides. In some embodiments, the one or more mRNA each comprise a 5′ Cap 1 structure.

In some embodiments, the one or more mRNA comprise at least one chemical modification. In one embodiment, the chemical modification is N1-methylpseudouridine. In another embodiment, the one or more mRNA is fully modified with N1-methylpseudouridine.

In some embodiments, the one or more mRNA encode 45-55 personalized cancer antigens. In one embodiment, the one or more mRNA encode 52 personalized cancer antigens. In some embodiments, each of the personalized cancer antigens is encoded by a separate open reading frame. In another embodiment, the peptide epitopes are in the form of a concatemeric cancer antigen comprised of 2-100 peptide epitopes, optionally wherein the concatemeric cancer antigen is comprised of 5-100 peptide epitopes.

In some embodiments, the concatemeric cancer antigen comprises one or more of: a) the 2-100 peptide epitopes, or the 5-100 peptide epitopes, are interspersed by cleavage sensitive sites; b) the mRNA encoding each peptide epitope is linked directly to one another without a linker; c) the mRNA encoding each peptide epitope is linked to one or another with a single nucleotide linker; d) each peptide epitope comprises 25-35 amino acids and includes a centrally located SNP mutation; e) at least 30% of the peptide epitopes have a highest affinity for class I MHC molecules from a subject; f) at least 30% of the peptide epitopes have a highest affinity for class II MHC molecules from a subject; g) at least 50% of the peptide epitopes have a predicated binding affinity of IC>500 nM for HLA-A, HLA-B and/or DRB1; h) the mRNA encodes 45-55 peptide epitopes; i) the mRNA encodes 52 peptide epitopes; j) 50% of the peptide epitopes have a binding affinity for class I MHC and 50% of the peptide epitopes have a binding affinity for class II MHC; k) the mRNA encoding the peptide epitopes is arranged such that the peptide epitopes are ordered to minimize pseudo-epitopes, 1) at least 30% of the peptide epitopes are class I MHC binding peptides of 15 amino acids in length; and/or m) at least 30% of the peptide epitopes are class II MHC binding peptides of 21 amino acids in length.

In some aspects, the disclosure provides an mRNA cancer vaccine comprising one or more mRNA each having one or more open reading frames encoding 45-55 peptide epitopes which are personalized cancer antigens formulated in a lipid nanoparticle.

In some aspects, the disclosure provides an mRNA cancer vaccine, comprising one or more mRNA each having one or more open reading frames encoding 45-55 peptide epitopes which are personalized cancer antigens formulated in a lipid nanoparticle; optionally wherein at least one of the peptide epitopes is an activating oncogene mutation peptide or a traditional cancer antigen, and optionally wherein at least three of the peptide epitopes are complex variants and at least two of the peptide epitopes are point mutations.

In some embodiments, the one or more mRNA encode 48-54 personalized cancer antigens. In one embodiment, the one or more mRNA encode 52 personalized cancer antigens. In some embodiments, each of the personalized cancer antigens is encoded by a separate open reading frame.

In another embodiment, the peptide epitopes are in the form of a concatemeric cancer antigen comprised of 2-100 peptide epitopes, optionally wherein the concatemeric cancer antigen is comprised of 5-100 peptide epitopes. In some embodiments, the concatemeric cancer antigen comprises one or more of: a) the 2-100 peptide epitopes, or the 5-100 peptide epitopes, are interspersed by cleavage sensitive sites; b) the mRNA encoding each peptide epitope is linked directly to one another without a linker; c) the mRNA encoding each peptide epitope is linked to one or another with a single nucleotide linker; d) each peptide epitope comprises 25-35 amino acids and includes a centrally located SNP mutation; e) at least 30% of the peptide epitopes have a highest affinity for class I MHC molecules from a subject; f) at least 30% of the peptide epitopes have a highest affinity for class II MHC molecules from a subject; g) at least 50% of the peptide epitopes have a predicated binding affinity of IC>500 nM for HLA-A, HLA-B and/or DRB1; h) the mRNA encodes 45-55 peptide epitopes; i) the mRNA encodes 52 peptide epitopes; j) 50% of the peptide epitopes have a binding affinity for class I MHC and 50% of the peptide epitopes have a binding affinity for class II MHC; k) the mRNA encoding the peptide epitopes is arranged such that the peptide epitopes are ordered to minimize pseudo-epitopes, 1) at least 30% of the peptide epitopes are class I MHC binding peptides of 15 amino acids in length; and/or m) at least 30% of the peptide epitopes are class II MHC binding peptides of 21 amino acids in length.

In some embodiments, at least two of the peptide epitopes are separated from one another by a universal type II T-cell epitope. In one embodiment, all of the peptide epitopes are separated from one another by a universal type II T-cell epitope. In another embodiment, the mRNA cancer vaccine encodes 1-20 universal type II T-cell epitopes.

In some embodiments, the universal type II T-cell epitope is selected from the group consisting of: ILMQYIKANSKFIGI (Tetanus toxin; SEQ ID NO: 226), FNNFTVSFWLRVPKVSASHLE, (Tetanus toxin; SEQ ID NO: 227), QYIKANSKFIGITE (Tetanus toxin; SEQ ID NO: 228) QSIALSSLMVAQAIP (Diptheria toxin; SEQ ID NO: 229), and AKFVAAWTLKAAA (pan-DR epitope; SEQ ID NO: 230).

In one embodiment, the universal type II T-cell epitope is the same universal type II T-cell epitope throughout the mRNA. In some embodiments, the universal type II T-cell epitope is repeated 1-20 times in the mRNA. In another embodiment, the universal type II T-cell epitopes are different from one another throughout the mRNA. In one embodiment, the universal type II T-cell epitope is located between every peptide epitope. In some embodiments, the universal type II T-cell epitope is located between every other peptide epitope. In one embodiment, the universal type II T-cell epitope is located between every third peptide epitope.

In some embodiments, the one or more mRNA further comprise an open reading frame encoding an immune potentiator. In one embodiment, the immune potentiator is formulated in the lipid nanoparticle. In another embodiment, the immune potentiator is formulated in a separate lipid nanoparticle. In some embodiments, the immune potentiator is a constitutively active human STING polypeptide. In one embodiment, the constitutively active human STING polypeptide comprises the amino acid sequence shown in SEQ ID NO: 1. In another embodiment, the mRNA encoding the constitutively active human STING polypeptide comprises the nucleotide sequence shown in SEQ ID NO: 170.

In some embodiments, one or more of the following conditions are met: (i) the activating oncogene mutation is a KRAS mutation; (ii) the KRAS mutation is a G12 mutation, optionally wherein the G12 KRAS mutation is selected from a G12D, G12V, G12S, G12C, G12A, and a G12R KRAS mutation; (iii) the KRAS mutation is a G13 mutation, optionally wherein the G13 KRAS mutation is a G13D KRAS mutation; and/or (iv) the activating oncogene mutation is a H-RAS or N-RAS mutation.

In certain embodiments, one or more of the following conditions are met: (A) the mRNA has an open reading frame encoding a concatemer of two or more activating oncogene mutation peptides; (B) at least two of the peptide epitopes are separated from one another by a single Glycine, optionally wherein all of the peptide epitopes are separated from one another by a single Glycine; (C) the concatemer comprises 3-10 activating oncogene mutation peptides; and/or (D) at least two of the peptide epitopes are linked directly to one another without a linker.

In specific embodiments, one or more of the following conditions are met: (i) at least one of the peptide epitopes is a traditional cancer antigen; (ii) at least one of the peptide epitopes is a recurrent polymorphism; (iii) the recurrent polymorphism comprises a recurrent somatic cancer mutation in p53; (iv) the recurrent somatic cancer mutation in p53 is selected from the group consisting of: (A) mutations at the canonical 5′ splice site neighboring codon p.T125, inducing a retained intron having peptide sequence TAKSVTCTVSCPEGLASMRLQCLAVSPCISFVWNFGIPLHPLASCQCFFIVYPLNV (SEQ ID NO: 232) that contains epitopes AVSPCISFVW (SEQ ID NO: 233) (HLA-B*57:01, HLA-B*58:01), HPLASCQCFF (SEQ ID NO: 234) (HLA-B*35:01, HLA-B*53:01), FVWNFGIPL (SEQ ID NO: 235) (HLA-A*02:01, HLA-A*02:06, HLA-B*35:01); (B) mutations at the canonical 5′ splice site neighboring codon p.331, inducing a retained intron having peptide sequence EYFTLQVLSLGTSYQVESFQSNTQNAVFFLTVLPAIGAFAIRGQ (SEQ ID NO: 236) that contains epitopes LQVLSLGTSY (SEQ ID NO: 237) (HLA-B*15:01), FQSNTQNAVF (SEQ ID NO: 238) (HLA-B*15:01); (C) mutations at the canonical 3′ splice site neighboring codon p.126, inducing a cryptic alternative exonic 3′ splice site producing the novel spanning peptide sequence AKSVTCTMFCQLAK (SEQ ID NO: 239) that contains epitopes CTMFCQLAK (SEQ ID NO: 240) (HLA-A*11:01), KSVTCTMF (SEQ ID NO: 241) (HLA-B*58:01); and/or (D) mutations at the canonical 5′ splice site neighboring codon p.224, inducing a cryptic alternative intronic 5′ splice site producing the novel spanning peptide sequence VPYEPPEVWLALTVPPSTAWAA (SEQ ID NO: 242) that contains epitopes VPYEPPEVW (SEQ ID NO: 243) (HLA-B*53:01, HLA-B*51:01), LTVPPSTAW (SEQ ID NO: 244) (HLA-B*58:01, HLA-B*57:01), wherein the transcript codon positions refer to the canonical full-length p53 transcript ENST00000269305 (SEQ ID NO: 245) from the Ensembl v83 human genome annotation; and/or (v) the mRNA cancer vaccine does not comprise a stabilizing agent.

Another aspect of the present disclosure is an mRNA cancer vaccine, comprising a lipid nanoparticle comprising (i) one or more mRNA each having one or more open reading frames encoding 1-500 peptide epitopes which are personalized cancer antigens, and (ii) an mRNA having an open reading frame encoding a polypeptide that enhances an immune response to the personalized cancer antigens, optionally wherein (i) and (ii) are present at mass ratio of approximately 5:1.

Another aspect of the present disclosure is an mRNA cancer vaccine, comprising: a lipid nanoparticle comprising: (i) one or more mRNA each having one or more open reading frames encoding 1-500 peptide epitopes which are personalized cancer antigens, and (ii) an mRNA having an open reading frame encoding a polypeptide that enhances an immune response to the personalized cancer antigens, optionally wherein (i) and (ii) are present at mass ratio of approximately 5:1; optionally wherein at least one of the peptide epitopes is an activating oncogene mutation peptide or a traditional cancer antigen, and optionally wherein at least three of the peptide epitopes are complex variants and at least two of the peptide epitopes are point mutations.

In some embodiments, the immune response comprises a cellular or humoral immune response characterized by: (i) stimulating Type I interferon pathway signaling; (ii) stimulating NFkB pathway signaling; (iii) stimulating an inflammatory response; (iv) stimulating cytokine production; or (v) stimulating dendritic cell development, activity or mobilization; and (vi) a combination of any of (i)-(vi).

In one embodiment, the mRNA cancer vaccine comprises a single mRNA construct encoding both the peptide epitopes and the polypeptide that enhances an immune response to the personalized cancer antigens. In another embodiment the peptide epitopes are in the form of a concatemeric cancer antigen comprised of 2-100 peptide epitopes, optionally wherein the concatemeric cancer antigen is comprised of 5-100 peptide epitopes.

In some embodiments, the concatemeric cancer antigen comprises one or more of: a) the 2-100 peptide epitopes, or the 5-100 peptide epitopes, are interspersed by cleavage sensitive sites; b) the mRNA encoding each peptide epitope is linked directly to one another without a linker; c) the mRNA encoding each peptide epitope is linked to one or another with a single nucleotide linker; d) each peptide epitope comprises 25-35 amino acids and includes a centrally located SNP mutation; e) at least 30% of the peptide epitopes have a highest affinity for class I MHC molecules from a subject; f) at least 30% of the peptide epitopes have a highest affinity for class II MHC molecules from a subject; g) at least 50% of the peptide epitopes have a predicated binding affinity of IC>500 nM for HLA-A, HLA-B and/or DRB1; h) the mRNA encodes 45-55 peptide epitopes; i) the mRNA encodes 52 peptide epitopes; j) 50% of the peptide epitopes have a binding affinity for class I MHC and 50% of the peptide epitopes have a binding affinity for class II MHC; k) the mRNA encoding the peptide epitopes is arranged such that the peptide epitopes are ordered to minimize pseudo-epitopes, 1) at least 30% of the peptide epitopes are class I MHC binding peptides of 15 amino acids in length; and/or m) at least 30% of the peptide epitopes are class II MHC binding peptides of 21 amino acids in length.

In some embodiments, each peptide epitope comprises a centrally located SNP mutation with 15 flanking amino acids on each side of the SNP mutation.

In one embodiment, the polypeptide that enhances an immune response to at least one personalized cancer antigens in a subject is a constitutively active human STING polypeptide. In one embodiment, the constitutively active human STING polypeptide comprises one or more mutations selected from the group consisting of V147L, N154S, V155M, R284M, R284K, R284T, E315Q, R375A, and combinations thereof. In another embodiment, the constitutively active human STING polypeptide comprises a V155M mutation. In another embodiment, the constitutively active human STING polypeptide comprises mutations R284M/V147L/N154S/V155M.

In some embodiments, each mRNA is formulated in the same or different lipid nanoparticle. In another embodiment, each mRNA encoding a cancer personalized cancer antigens is formulated in the same or different lipid nanoparticle. In some embodiments, each mRNA encoding a polypeptide that enhances an immune response to the personalized cancer antigens is formulated in the same or different lipid nanoparticle.

In some embodiments, each mRNA encoding a personalized cancer antigen is formulated in the same lipid nanoparticle, and each mRNA encoding a polypeptide that enhances an immune response to the personalized cancer antigen is formulated in a different lipid nanoparticle. In another embodiment, each mRNA encoding a personalized cancer antigen is formulated in the same lipid nanoparticle, and each mRNA encoding a polypeptide that enhances an immune response to the personalized cancer antigen is formulated in the same lipid nanoparticle as each mRNA encoding a personalized cancer antigen. In some embodiments, each mRNA encoding a personalized cancer antigen is formulated in a different lipid nanoparticle, and each mRNA encoding a polypeptide that enhances an immune response to the personalized cancer antigen is formulated in the same lipid nanoparticle as each mRNA encoding each personalized cancer antigen.

In some embodiments, the peptide epitopes are T cell epitopes and/or B cell epitopes.

In other embodiments, the peptide epitopes comprise a combination of T cell epitopes and B cell epitopes. In one embodiment, at least 1 of the peptide epitopes is a T cell epitope. In another embodiment, at least 1 of the peptide epitopes is a B cell epitope.

In some embodiments, the peptide epitopes have been optimized for binding strength to a MHC of the subject. In other embodiments, a TCR face for each epitope has a low similarity to endogenous proteins.

In another embodiment, the mRNA cancer vaccine further comprises a recall antigen. In some embodiments, the recall antigen is an infectious disease antigen.

In one embodiment, the mRNA cancer vaccine further comprises an mRNA having an open reading frame encoding one or more traditional cancer antigens.

In one embodiment, one or more of the following conditions are met: (i) the activating oncogene mutation is a KRAS mutation; (ii) the KRAS mutation is a G12 mutation, optionally wherein the G12 KRAS mutation is selected from a G12D, G12V, G12S, G12C, G12A, and a G12R KRAS mutation; (iii) the KRAS mutation is a G13 mutation, optionally wherein the G13 KRAS mutation is a G13D KRAS mutation; and/or (iv) the activating oncogene mutation is a H-RAS or N-RAS mutation.

In one embodiment, one or more of the following conditions are met: (A) the mRNA has an open reading frame encoding a concatemer of two or more activating oncogene mutation peptides; (B) at least two of the peptide epitopes are separated from one another by a single Glycine, optionally wherein all of the peptide epitopes are separated from one another by a single Glycine; (C) the concatemer comprises 3-10 activating oncogene mutation peptides; and/or (D) at least two of the peptide epitopes are linked directly to one another without a linker.

In one embodiment, one or more of the following conditions are met: (i) at least one of the peptide epitopes is a traditional cancer antigen; (ii) at least one of the peptide epitopes is a recurrent polymorphism; (iii) the recurrent polymorphism comprises a recurrent somatic cancer mutation in p53; (iv) the recurrent somatic cancer mutation in p53 is selected from the group consisting of: (A) mutations at the canonical 5′ splice site neighboring codon p.T125, inducing a retained intron having peptide sequence TAKSVTCTVSCPEGLASMRLQCLAVSPCISFVWNFGIPLHPLASCQCFFIVYPLNV (SEQ ID NO: 232) that contains epitopes AVSPCISFVW (SEQ ID NO: 233) (HLA-B*57:01, HLA-B*58:01), HPLASCQCFF (SEQ ID NO: 234) (HLA-B*35:01, HLA-B*53:01), FVWNFGIPL (SEQ ID NO: 235) (HLA-A*02:01, HLA-A*02:06, HLA-B*35:01); (B) mutations at the canonical 5′ splice site neighboring codon p.331, inducing a retained intron having peptide sequence YFTLQVLSLGTSYQVESFQSNTQNAVFFLTVLPAIGAFAIRGQ (SEQ ID NO: 236) that contains epitopes LQVLSLGTSY (SEQ ID NO: 237) (HLA-B*15:01), FQSNTQNAVF (SEQ ID NO: 238) (HLA-B*15:01); (C) mutations at the canonical 3′ splice site neighboring codon p.126, inducing a cryptic alternative exonic 3′ splice site producing the novel spanning peptide sequence AKSVTCTMFCQLAK (SEQ ID NO: 239) that contains epitopes CTMFCQLAK (SEQ ID NO: 240) (HLA-A*11:01), KSVTCTMF (SEQ ID NO: 241) (HLA-B*58:01); and/or (D) mutations at the canonical 5′ splice site neighboring codon p.224, inducing a cryptic alternative intronic 5′ splice site producing the novel spanning peptide sequence VPYEPPEVWLALTVPPSTAWAA (SEQ ID NO: 242) that contains epitopes VPYEPPEVW (SEQ ID NO: 243) (HLA-B*53:01, HLA-B*51:01), LTVPPSTAW (SEQ ID NO: 244) (HLA-B*58:01, HLA-B*57:01), wherein the transcript codon positions refer to the canonical full-length p53 transcript ENST00000269305 (SEQ ID NO: 245) from the Ensembl v83 human genome annotation; and/or (v) the mRNA cancer vaccine does not comprise a stabilizing agent.

In some embodiments, the lipid nanoparticle comprises a molar ratio of about 20-60% ionizable amino lipid:5-25% neutral lipid:25-55% sterol; and 0.5-15% PEG-modified lipid, optionally wherein the ionizable amino lipid is a cationic lipid. In one embodiment, the lipid nanoparticle comprises a molar ratio of about 50% compound 25:about 10% DSPC:about 38.5% cholesterol; and about 1.5% PEG-DMG. In another embodiment, the ionizable amino lipid is selected from the group consisting of for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319). In some embodiments, the lipid nanoparticle comprises a compound of Formula (I). In one embodiment, the compound of Formula (I) is Compound 25. In another embodiment, the lipid nanoparticle has a polydispersity value of less than 0.4. In some embodiments, the lipid nanoparticle has a net neutral charge at a neutral pH value.

In one embodiment, a TCR face for each epitope has a low similarity to endogenous proteins.

In another embodiment, the mRNA further comprises an open reading frame encoding an immune checkpoint modulator. In one embodiment, the mRNA cancer vaccine further comprises an additional cancer therapeutic agent; optionally wherein the additional cancer therapeutic agent is an immune checkpoint modulator. In another embodiment, the immune checkpoint modulator is an inhibitory checkpoint polypeptide. In some embodiments, the inhibitory checkpoint polypeptide inhibits PD1, PD-L1, CTLA4, TIM-3, VISTA, A2AR, B7-H3, B7-H4, BTLA, IDO, KIR, LAG3, or a combination thereof.

In some embodiments, the checkpoint inhibitor polypeptide is an antibody. In one embodiment, the inhibitory checkpoint polypeptide is an antibody selected from an anti-CTLA4 antibody or antigen-binding fragment thereof that specifically binds CTLA4, an anti-PD1 antibody or antigen-binding fragment thereof that specifically binds PD, an anti-PD-L1 antibody or antigen-binding fragment thereof that specifically binds PD-L1, and a combination thereof. In one embodiment, the checkpoint inhibitor polypeptide is an anti-PD-L1 antibody selected from atezolizumab, avelumab, or durvalumab. In another embodiment, the checkpoint inhibitor polypeptide is an anti-CTLA-4 antibody selected from tremelimumab or ipilimumab. In some embodiments, the checkpoint inhibitor polypeptide is an anti-PD1 antibody selected from nivolumab or pembrolizumab.

In some embodiments, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

The present disclosure, in another aspect, provides a method for vaccinating a subject, comprising administering to a subject having cancer the mRNA cancer vaccine described above.

In some embodiments, the mRNA vaccine is administered at a dosage level sufficient to deliver between 10 μg and 400 μg of the mRNA vaccine to the subject. In one embodiment, the mRNA vaccine is administered at a dosage level sufficient to deliver 0.033 mg, 0.1 mg, 0.2 mg, or 0.4 mg to the subject. In another embodiment, the mRNA vaccine is administered to the subject twice, three times, four times or more. In some embodiments, the mRNA vaccine is administered once a day every three weeks. In one embodiment, the mRNA vaccine is administered by intradermal, intramuscular, and/or subcutaneous administration. In another embodiment, the mRNA vaccine is administered by intramuscular administration.

In some embodiments, the method further comprises administering an additional cancer therapeutic agent; optionally wherein the additional cancer therapeutic agent is an immune checkpoint modulator to the subject. In one embodiment, the immune checkpoint modulator is an inhibitory checkpoint polypeptide. In another embodiment, the inhibitory checkpoint polypeptide inhibits PD1, PD-L1, CTLA4, TIM-3, VISTA, A2AR, B7-H3, B7-H4, BTLA, IDO, KIR, LAG3, or a combination thereof. In some embodiments, the checkpoint inhibitor polypeptide is an antibody. In other embodiments, the inhibitory checkpoint polypeptide is an antibody selected from an anti-CTLA4 antibody or antigen-binding fragment thereof that specifically binds CTLA4, an anti-PD 1 antibody or antigen-binding fragment thereof that specifically binds PD1, an anti-PD-L1 antibody or antigen-binding fragment thereof that specifically binds PD-L 1, and a combination thereof. In some embodiments, the checkpoint inhibitor polypeptide is an anti-PD-L1 antibody selected from atezolizumab, avelumab, or durvalumab. In another embodiment, the checkpoint inhibitor polypeptide is an anti-CTLA-4 antibody selected from tremelimumab or ipilimumab. In other embodiments, the checkpoint inhibitor polypeptide is an anti-PD1 antibody selected from nivolumab or pembrolizumab.

In one embodiment, the immune checkpoint modulator is administered at a dosage level sufficient to deliver 100-300 mg to the subject. In some embodiments, the immune checkpoint modulator is administered at a dosage level sufficient to deliver 200 mg to the subject. In some embodiments, the immune checkpoint modulator is administered by intravenous infusion. In one embodiment, the immune checkpoint modulator is administered to the subject twice, three times, four times or more. In some embodiments, the immune checkpoint modulator is administered to the subject on the same day as the mRNA vaccine administration.

In some embodiments, the cancer is selected from the group consisting of non-small cell lung cancer (NSCLC), small cell lung cancer, melanoma, bladder urothelial carcinoma, HPV-negative head and neck squamous cell carcinoma (HNSCC), and a solid malignancy that is microsatellite high (MSI H)/mismatch repair (MMR) deficient. In one embodiment, the NSCLC lacks an EGFR sensitizing mutation and/or an ALK translocation. In another embodiment, the solid malignancy that is microsatellite high (MSI H)/mismatch repair (MMR) deficient is selected from the group consisting of colorectal cancer, stomach adenocarcinoma, esophageal adenocarcinoma, and endometrial cancer. In some embodiments, the cancer is selected from cancer of the pancreas, peritoneum, large intestine, small intestine, biliary tract, lung, endometrium, ovary, genital tract, gastrointestinal tract, cervix, stomach, urinary tract, colon, rectum, and hematopoietic and lymphoid tissues.

The invention in some aspects is an mRNA cancer vaccine of one or more mRNA each having an open reading frame encoding a cancer antigen peptide epitope formulated in a lipid nanoparticle, wherein the mRNA vaccine encodes 5-100 peptide epitopes and at least two of the peptide epitopes are personalized cancer antigens, and a pharmaceutically acceptable carrier or excipient.

In other aspects the invention is an mRNA cancer vaccine, having one or more mRNA each having an open reading frame encoding a cancer antigen peptide epitope, wherein the mRNA vaccine encodes 5-100 peptide epitopes and at least three of the peptide epitopes is a complex variant and at least two of the peptide epitopes are point mutations, and a pharmaceutically acceptable carrier or excipient.

In some embodiments, the lipid nanoparticle comprises a molar ratio of about 20-60% cationic lipid:5-25% non-cationic lipid:25-55% sterol; and 0.5-15% PEG-modified lipid. In some embodiments, the cationic lipid is selected from the group consisting of for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319). In other embodiments, the lipid nanoparticle comprises a compound of Formula (I). In some embodiments, the compound of Formula (I) is Compound 25.

In some embodiments, the lipid nanoparticle has a polydispersity value of less than 0.4. In some embodiments, the lipid nanoparticle has a net neutral charge at a neutral pH value.

The vaccine in some embodiments is an mRNA having an open reading frame encoding a concatemeric cancer antigen comprised of the 5-100 peptide epitopes. In other embodiments at least two of the peptide epitopes are separated from one another by a single Glycine. In other embodiments the concatemeric cancer antigen comprises 20-40 peptide epitopes. In some embodiments all of the peptide epitopes are separated from one another by a single Glycine. In some embodiments at least two of the peptide epitopes are linked directly to one another without a linker.

Each peptide epitope in embodiments comprises a 25-35 amino acids and includes a centrally located SNP mutation.

In some embodiments at least 30% of the peptide epitopes have a highest affinity for class I MHC molecules from the subject. In other embodiments at least 30% of the peptide epitopes have a highest affinity for class II MHC molecules from the subject. In yet other embodiments at least 50% of the peptide epitopes have a predicted binding affinity of IC>500 nM for HLA-A, HLA-B and/or DRB1.

In some embodiments, one or more mRNAs of the invention encode up to 20 peptide epitopes. In some embodiments, one or more mRNAs of the invention encode up to 50 epitopes. In some embodiments, one or more mRNAs of the invention encode up to 100 epitopes.

According to other embodiments the mRNA encoding the peptide epitopes is arranged such that the peptide epitopes are ordered to minimize pseudo-epitopes.

Each peptide epitope may comprise 31 amino acids and includes a centrally located SNP mutation with 15 flanking amino acids on each side of the SNP mutation.

In some embodiments a TCR face for each epitope has a low similarity to endogenous proteins.

In yet other embodiments the mRNA further comprises a recall antigen. The recall antigen may be an infectious disease antigen.

In other embodiments, at least one of the peptide epitopes is a traditional cancer antigen. The vaccine in some embodiments includes an mRNA having an open reading frame encoding one or more recurrent polymorphisms. The one or more recurrent polymorphisms may comprise a recurrent somatic cancer mutation in p53. The one or more recurrent somatic cancer mutation in p53 in some embodiments are selected from the group consisting of: (A) mutations at the canonical 5′ splice site neighboring codon p.T125, inducing a retained intron having peptide sequence TAKSVTCTVSCPEGLASMRLQCLAVSPCISFVWNFGIPLHPLASCQCFFIVYPLNV (SEQ ID NO: 232) that contains epitopes AVSPCISFVW (SEQ ID NO: 233) (HLA-B*57:01, HLA-B*58:01), HPLASCQCFF (SEQ ID NO: 234) (HLA-B*35:01, HLA-B*53:01), FVWNFGIPL (SEQ ID NO: 235) (HLA-A*02:01, HLA-A*02:06, HLA-B*35:01); (B) mutations at the canonical 5′ splice site neighboring codon p.331, inducing a retained intron having peptide sequence EYFTLQVLSLGTSYQVESFQSNTQNAVFFLTVLPAIGAFAIRGQ (SEQ ID NO: 236) that contains epitopes LQVLSLGTSY (SEQ ID NO: 237) (HLA-B*15:01), FQSNTQNAVF (SEQ ID NO: 238) (HLA-B*15:01); (C) mutations at the canonical 3′ splice site neighboring codon p.126, inducing a cryptic alternative exonic 3′ splice site producing the novel spanning peptide sequence AKSVTCTMFCQLAK (SEQ ID NO: 239) that contains epitopes CTMFCQLAK (SEQ ID NO: 240) (HLA-A*11:01), KSVTCTMF (SEQ ID NO: 241) (HLA-B*58:01); and/or (D) mutations at the canonical 5′ splice site neighboring codon p.224, inducing a cryptic alternative intronic 5′ splice site producing the novel spanning peptide sequence VPYEPPEVWLALTVPPSTAWAA (SEQ ID NO: 242) that contains epitopes VPYEPPEVW (SEQ ID NO: 243) (HLA-B*53:01, HLA-B*51:01), LTVPPSTAW (SEQ ID NO: 244) (HLA-B*58:01, HLA-B*57:01), wherein the transcript codon positions refer to the canonical full-length p53 transcript ENST00000269305 (SEQ ID NO: 245) from the Ensembl v83 human genome annotation.

In some embodiments, the mRNA further comprises an open reading frame encoding an immune checkpoint modulator. In some embodiments, the mRNA cancer vaccine comprises an immune checkpoint modulator. In some embodiments, the immune checkpoint modulator is an inhibitory checkpoint polypeptide. In some embodiments, the inhibitory checkpoint polypeptide is an antibody or fragment thereof that specifically binds to a molecule selected from the group consisting of PD-1, TIM-3, VISTA, A2AR, B7-H3, B7-H4, BTLA, CTLA-4, IDO, KIR and LAG3. In some embodiments, the inhibitory checkpoint polypeptide is an anti-CTLA4 or anti-PD1 antibody. In some embodiments, the anti-PD-1 antibody is pembrolizumab.

In some embodiments, the mRNA cancer vaccine does not comprise a stabilization agent.

In some embodiments the mRNA includes at least one chemical modification. The chemical modification may be selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In other aspects a method for vaccinating a subject is provided. The method involves administering to a subject having cancer an mRNA vaccine disclosed herein.

In some embodiments, the mRNA vaccine is administered at a dosage level sufficient to deliver between 10 μg and 400 μg of the mRNA vaccine to the subject. In some embodiments, the mRNA vaccine is administered at a dosage level sufficient to deliver 0.033 mg, 0.1 mg, 0.2 mg, or 0.4 mg to the subject. In some embodiments, the mRNA vaccine is administered to the subject twice, three times, four times or more. In some embodiments, the mRNA vaccine is administered once a day every three weeks.

In some embodiments, the mRNA vaccine is administered by intradermal, intramuscular, and/or subcutaneous administration. In some embodiments, the mRNA vaccine is administered by intramuscular administration.

In some embodiments, the method further includes administering an additional cancer therapeutic agent; optionally wherein the additional cancer therapeutic agent is an immune checkpoint modulator to the subject. In some embodiments, the immune checkpoint modulator is an inhibitory checkpoint polypeptide. In some embodiments, the inhibitory checkpoint polypeptide is an antibody or fragment thereof that specifically binds to a molecule selected from the group consisting of PD-1, TIM-3, VISTA, A2AR, B7-H3, B7-H4, BTLA, CTLA-4, IDO, KIR and LAG3. In some embodiments, the inhibitory checkpoint polypeptide is an anti-PD1 antibody. In some embodiments, the anti-PD-1 antibody is pembrolizumab.

In some embodiments, the immune checkpoint modulator is administered at a dosage level sufficient to deliver 100-300 mg to the subject. In some embodiments, the immune checkpoint modulator is administered at a dosage level sufficient to deliver 200 mg to the subject.

In some embodiments, the immune checkpoint modulator is administered by intravenous infusion.

In some embodiments, the immune checkpoint modulator is administered to the subject twice, three times, four times or more. In some embodiments, the immune checkpoint modulator is administered to the subject on the same day as the mRNA vaccine administration.

In some embodiments, the cancer is selected from the group consisting of non-small cell lung cancer (NSCLC), small cell lung cancer, melanoma, bladder urothelial carcinoma, HPV-negative head and neck squamous cell carcinoma (HNSCC), and a solid malignancy that is microsatellite high (MSI H)/mismatch repair (MMR) deficient. In some embodiments, the NSCLC lacks an EGFR sensitizing mutation and/or an ALK translocation. In some embodiments, the solid malignancy that is microsatellite high (MSI H)/mismatch repair (MMR) deficient is selected from the group consisting of colorectal cancer, stomach adenocarcinoma, esophageal adenocarcinoma, and endometrial cancer. In some embodiments, the cancer is selected from cancer of the pancreas, peritoneum, large intestine, small intestine, biliary tract, lung, endometrium, ovary, genital tract, gastrointestinal tract, cervix, stomach, urinary tract, colon, rectum, and hematopoietic and lymphoid tissues.

A method for preparing an mRNA cancer vaccine is provided in other aspects. The method involves isolating a sample from a subject, identifying a plurality of cancer antigens in the sample, determining immunogenic epitopes from the plurality of cancer antigens, preparing an mRNA cancer vaccine having an open reading frame encoding the cancer antigens. A method of producing an mRNA encoding a concatemeric cancer antigen comprising between 1000 and 3000 nucleotides, is provided in other aspects of the invention. The method involves

(a) binding a first polynucleotide comprising an open reading frame encoding the cancer antigen of any one of the preceding claims and a second polynucleotide comprising a 5′-UTR to a polynucleotide conjugated to a solid support;

(b) ligating the 3′-terminus of the second polynucleotide to the 5′-terminus of the first polynucleotide under suitable conditions, wherein the suitable conditions comprise a DNA Ligase, thereby producing a first ligation product;

(c) ligating the 5′ terminus of a third polynucleotide comprising a 3′-UTR to the 3′-terminus of the first ligation product under suitable conditions, wherein the suitable conditions comprise an RNA Ligase, thereby producing a second ligation product; and

(d) releasing the second ligation product from the solid support,

thereby producing an mRNA encoding the concatemeric cancer antigen comprising between 1000 and 3000 nucleotides.

In other aspects the invention is an mRNA cancer vaccine comprising a concatemeric cancer antigen preparable according to the methods described herein.

A method for treating a subject with a personalized mRNA cancer vaccine is provided according to other aspects of the invention. The method involves identifying a set of neoepitopes by analyzing a patient transcriptome and/or a patient exome from the sample to produce a patient specific mutanome, selecting a set of neoepitopes for the vaccine from the mutanome based on MHC binding strength, MHC binding diversity, predicted degree of immunogenicity, low self reactivity, presence of activating oncogene mutations, and/or T cell reactivity, preparing the mRNA vaccine to encode the set of neoepitopes, and administering the mRNA vaccine to the subject within two months of isolating the sample from the subject. In some embodiments, the identifying comprises analyzing a patient transcriptome and/or a patient exome from a sample from the subject. In some embodiments, the sample from the subject is a biological sample, e.g., a biopsy. In some embodiments, the method further comprises isolating the sample from the subject. In some embodiments, the identifying comprises analyzing tissue-specific expression in available databases.

A method of identifying a set of neoepitopes for use in a personalized mRNA cancer vaccine having one or more polynucleotides that encode the set of neoepitopes is provided in other aspects of the invention. The method involves:

a. identifying a patient specific mutanome by analyzing a patient transcriptome and a patient exome,

b. selecting a subset of 15-500 neoepitopes from the mutanome using a weighted value for the neoepitopes based on at least three of: an assessment of gene or transcript-level expression in patient RNA-seq; variant call confidence score; RNA-seq allele-specific expression; conservative vs. non-conservative amino acid substitution; position of point mutation (Centering Score for increased TCR engagement); position of point mutation (Anchoring Score for differential HLA binding); Selfness: <100% core epitope homology with patient WES data; HLA-A and -B IC50 for 8 mers-11 mers; HLA-DRB1 IC50 for 15 mers-20 mers; promiscuity Score (i.e. number of patient HLAs predicted to bind); HLA-C IC50 for 8 mers-11 mers; HLA-DRB3-5 IC50 for 15 mers-20 mers; HLA-DQB1/A1 IC50 for 15 mers-20 mers; HLA-DPB1/A1 IC50 for 15 mers-20 mers; Class I vs Class II proportion; Diversity of patient HLA-A, -B and DRB1 allotypes covered; proportion of point mutation vs complex epitopes (e.g. frameshifts); pseudo-epitope HLA binding scores; presence and/or abundance of RNAseq reads, and

c. selecting the set of neoepitopes for use in a personalized mRNA cancer vaccine from the subset based on the highest weighted value, wherein the set of neoepitopes comprise 15-40 neoepitopes.

The invention in some aspects is an mRNA cancer vaccine of one or more mRNA each having an open reading frame encoding a cancer antigen peptide epitope, wherein the mRNA the further comprises a miRNA binding site. In some embodiment the vaccine encodes 5-100 peptide epitopes.

In some embodiments the nucleic acid vaccines described herein are chemically modified. In other embodiments the nucleic acid vaccines are unmodified.

Yet other aspects provide compositions for and methods of vaccinating a subject comprising administering to the subject a nucleic acid vaccine comprising one or more RNA polynucleotides having an open reading frame encoding a cancer antigen epitope, wherein the RNA polynucleotide does not include a stabilization element, and wherein an adjuvant is not coformulated or co-administered with the vaccine.

In other aspects the invention is a composition for or method of vaccinating a subject comprising administering to the subject a nucleic acid vaccine comprising one or more RNA polynucleotides having an open reading frame encoding a first cancer antigen epitope wherein a dosage of between 10 μg/kg and 400 μg/kg of the nucleic acid vaccine is administered to the subject. In some embodiments the dosage of the RNA polynucleotide is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the nucleic acid vaccine is administered to the subject by intradermal or intramuscular injection. In some embodiments, the nucleic acid vaccine is administered to the subject on day zero. In some embodiments, a second dose of the nucleic acid vaccine is administered to the subject on day twenty one.

In some embodiments, a dosage of 25 micrograms of the RNA polynucleotide is included in the nucleic acid vaccine administered to the subject. In some embodiments, a dosage of 100 micrograms of the RNA polynucleotide is included in the nucleic acid vaccine administered to the subject. In some embodiments, a dosage of 50 micrograms of the RNA polynucleotide is included in the nucleic acid vaccine administered to the subject. In some embodiments, a dosage of 75 micrograms of the RNA polynucleotide is included in the nucleic acid vaccine administered to the subject. In some embodiments, a dosage of 150 micrograms of the RNA polynucleotide is included in the nucleic acid vaccine administered to the subject. In some embodiments, a dosage of 400 micrograms of the RNA polynucleotide is included in the nucleic acid vaccine administered to the subject. In some embodiments, a dosage of 200 micrograms of the RNA polynucleotide is included in the nucleic acid vaccine administered to the subject. In some embodiments, the RNA polynucleotide accumulates at a 100 fold higher level in the local lymph node in comparison with the distal lymph node. In other embodiments the nucleic acid vaccine is chemically modified and in other embodiments the nucleic acid vaccine is not chemically modified.

In some embodiments, the effective amount is a total dose of 1-100 μg. In some embodiments, the effective amount is a total dose of 100 μg. In some embodiments, the effective amount is a dose of 25 μg administered to the subject a total of one or two times. In some embodiments, the effective amount is a dose of 100 μg administered to the subject a total of two times. In some embodiments, the effective amount is a dose of 1 μg-10 μg, 1 μg-20 μg, 1 μg-30 μg, 5 μg-10 μg, 5 μg-20 μg, 5 μg-30 μg, 5 μg-40 μg, 5 μg-50 μg, 10 μg-15 μg, 10 μg-20 μg, 10 μg-25 μg, 10 μg-30 μg, 10 μg-40 μg, 10 μg-50 μg, 10 μg-60 μg, 30 μg-20 μg, 15 μg-25 μg, 15 μg-30 μg, 15 μg-40 μg, 15 μg-50 μg, 20 μg-25 μg, 20 μg-30 μg, 20 μg-40 μg 20 μg-50 μg, 20 μg-60 μg, 20 μg-70 μg, 20 μg-75 g, 30 μg-35 μg, 30 μg-40 μg, 30 μg-45 μg 30 μg-50 μg, 30 μg-60 μg, 30 μg-70 μg, 30 μg-75 μg which may be administered to the subject a total of one or two times or more.

Aspects of the invention provide a nucleic acid vaccine comprising one or more RNA polynucleotides having an open reading frame encoding a first antigenic polypeptide, wherein the RNA polynucleotide does not include a stabilization element, and a pharmaceutically acceptable carrier or excipient, wherein an adjuvant is not included in the vaccine. In some embodiments, the stabilization element is a histone stem-loop. In some embodiments, the stabilization element is a nucleic acid sequence having increased GC content relative to wild type sequence.

Aspects provide nucleic acid vaccines comprising one or more RNA polynucleotides having an open reading frame comprising at least one chemical modification or optionally no chemical modification, the open reading frame encoding a first antigenic polypeptide, wherein the RNA polynucleotide is present in the formulation for in vivo administration to a subject such that the level of antigen expression in the subject significantly exceeds a level of antigen expression produced by an mRNA vaccine having a stabilizing element or formulated with an adjuvant and encoding the first antigenic polypeptide.

Other aspects provide nucleic acid vaccines comprising one or more RNA polynucleotides having an open reading frame comprising at least one chemical modification or optionally no chemical modification, the open reading frame encoding a first antigenic polypeptide, wherein the vaccine has at least 10 fold less RNA polynucleotide than is required for an unmodified mRNA vaccine to produce an equivalent antibody titer.

Aspects of the invention also provide a unit of use vaccine, comprising between 10 ug and 400 ug of one or more RNA polynucleotides having an open reading frame comprising at least one chemical modification or optionally no chemical modification, the open reading frame encoding a first antigenic polypeptide, and a pharmaceutically acceptable carrier or excipient, formulated for delivery to a human subject. In some embodiments, the vaccine further comprises a cationic lipid nanoparticle.

Aspects of the invention provide kits including a vial comprising the mRNA cancer vaccine disclosed herein. In some embodiments, the vial contains 0.1 mg to 1 mg of mRNA. In some embodiments, the vial contains 0.35 mg of mRNA. In some embodiments, the concentration of the mRNA is 1 mg/mL.

In some embodiments, the vial contains 5-15 mg of total lipid. In some embodiments, the vial contains 7 mg of total lipid. In some embodiments, the concentration of total lipid is 20 mg/mL.

In some embodiments, the mRNA cancer vaccine is a liquid.

In some embodiments, the kit further includes a syringe. In some embodiments, the syringe is suitable for intramuscular administration.

Aspects of the invention provide methods of vaccinating a subject comprising administering to the subject a single dosage of between 25 ug/kg and 400 ug/kg of a nucleic acid vaccine comprising one or more RNA polynucleotides having an open reading frame encoding a first antigenic polypeptide in an effective amount to vaccinate the subject.

The invention in some aspects is an mRNA cancer vaccine which may include an activating oncogene mutation as an antigen. In some embodiments, the activating oncogene mutation is a KRAS mutation. In some embodiments, the KRAS mutation is a G12 mutation. In some embodiments, the G12 KRAS mutation is selected from a G12D, G12V, G12S, G12C, G12A, and a G12R KRAS mutation, e.g., the G12 KRAS mutation is selected from a G12D, G12V, and a G12S KRAS mutation. In other embodiments, the KRAS mutation is a G13 mutation, e.g., the G13 KRAS mutation is a G13D KRAS mutation. In some embodiments, the activating oncogene mutation is a H-RAS or N-RAS mutation.

In some embodiments the skilled artisan will select a KRAS mutation, a HLA subtype and a tumor type based on the guidance provided herein and prepare a KRAS vaccine for therapy. In some embodiments the KRAS mutations is selected from: G12C, G12V, G12D, G13D. In some embodiments the HLA subtype is selected from: A*02:01, C*07:01, C*04:01, C*07:02. In some embodiments the tumor type is selected from colorectal, pancreatic, lung, and endometrioid.

In some embodiments, the HRAS mutation is a mutation at codon 12, codon 13, or codon 61. In some embodiments, the HRAS mutation is a 12V, 61L, or 61R mutation.

In some embodiments, the NRAS mutation is a mutation at codon 12, codon 13, or codon 61. In some embodiments, the NRAS mutation is a 12D, 13D, 61K, or 61R mutation.

Some embodiments of the present disclosure provide an mRNA cancer vaccine that include an mRNA having an open reading frame encoding a concatemer of two or more activating oncogene mutation peptides. In some embodiments, at least two of the peptide epitopes are separated from one another by a single Glycine. In some embodiments, the concatemer comprises 3-10 activating oncogene mutation peptides. In some such embodiments, all of the peptide epitopes are separated from one another by a single Glycine. In other embodiments, at least two of the peptide epitopes are linked directly to one another without a linker.

In some embodiments, the mRNA cancer vaccine further comprises a cancer therapeutic agent. In some embodiments, the mRNA cancer vaccine further comprises an inhibitory checkpoint polypeptide. For example, in some embodiments, the inhibitory checkpoint polypeptide is an antibody or fragment thereof that specifically binds to a molecule selected from the group consisting of PD-1, TIM-3, VISTA, A2AR, B7-H3, B7-H4, BTLA, CTLA-4, IDO, KIR and LAG3. In other embodiments, the mRNA cancer vaccine further comprises a recall antigen. For example, in some embodiments, the recall antigen is an infectious disease antigen.

In some embodiments, the mRNA cancer vaccine does not comprise a stabilization agent.

In some embodiments the mRNA is formulated in a lipid nanoparticle carrier such as a lipid nanoparticle carrier comprising a molar ratio of about 20-60% cationic lipid:5-25% non-cationic lipid:25-55% sterol; and 0.5-15% PEG-modified lipid. The cationic lipid may be selected from the group consisting of for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

In some embodiments the mRNA includes at least one chemical modification. The chemical modification may be selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In other aspects, a method for treating a subject is provided. The method involves administering to a subject having cancer an mRNA cancer vaccine of any one of the foregoing embodiments. In some embodiments, the mRNA cancer vaccine is administered in combination with a cancer therapeutic agent. In some embodiments, the mRNA cancer vaccine is administered in combination with an inhibitory checkpoint polypeptide. For example, in some embodiments, the mRNA cancer vaccine is an antibody or fragment thereof that specifically binds to a molecule selected from the group consisting of PD-1, TIM-3, VISTA, A2AR, B7-H3, B7-H4, BTLA, CTLA-4, IDO, KIR and LAG3.

Methods provided herein may be used for treating a subject having cancer. In some embodiments, the cancer is selected from cancer of the pancreas, peritoneum, large intestine, small intestine, biliary tract, lung, endometrium, ovary, genital tract, gastrointestinal tract, cervix, stomach, urinary tract, colon, rectum, and hematopoietic and lymphoid tissues. In some embodiments, the cancer is colorectal cancer.

In some embodiments the dosage of the mRNA cancer vaccine administered to a subject is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the mRNA cancer vaccine is administered to the subject by intradermal or intramuscular injection. In some embodiments, the mRNA cancer vaccine is administered to the subject on day zero. In some embodiments, a second dose of the mRNA cancer vaccine is administered to the subject on day twenty one.

In some embodiments, a dosage of 25 micrograms of the mRNA cancer vaccine is administered to the subject. In some embodiments, a dosage of 100 micrograms of the mRNA cancer vaccine is administered to the subject. In some embodiments, a dosage of 50 micrograms of the mRNA cancer vaccine is administered to the subject. In some embodiments, a dosage of 75 micrograms of the mRNA cancer vaccine is administered to the subject. In some embodiments, a dosage of 150 micrograms of the mRNA cancer vaccine is administered to the subject. In some embodiments, a dosage of 400 micrograms of the mRNA cancer vaccine is administered to the subject. In some embodiments, a dosage of 200 micrograms of the mRNA cancer vaccine is administered to the subject. In some embodiments, the mRNA cancer vaccine accumulates at a 100 fold higher level in the local lymph node in comparison with the distal lymph node. In other embodiments the mRNA cancer vaccine is chemically modified and in other embodiments the mRNA cancer vaccine is not chemically modified.

In some embodiments, the effective amount is a total dose of 1-100 μg. In some embodiments, the effective amount is a total dose of 100 μg. In some embodiments, the effective amount is a dose of 25 μg administered to the subject a total of one or two times. In some embodiments, the effective amount is a dose of 100 μg administered to the subject a total of two times. In some embodiments, the effective amount is a dose of 1 μg-10 μg, 1 μg-20 μg, 1 μg-30 μg, 5 μg-10 μg, 5 μg-20 μg, 5 μg-30 μg, 5 μg-40 μg, 5 μg-50 μg, 10 μg-15 μg, 10 μg-20 μg, 10 μg-25 μg, 10 μg-30 μg, 10 μg-40 μg, 10 μg-50 μg, 10 μg-60 μg, 30 μg-20 μg, 15 μg-25 μg, 15 μg-30 μg, 15 μg-40 μg, 15 μg-50 μg, 20 μg-25 μg, 20 μg-30 μg, 20 μg-40 μg 20 μg-50 μg, 20 μg-60 μg, 20 μg-70 μg, 20 μg-75 g, 30 μg-35 μg, 30 μg-40 μg, 30 μg-45 μg 30 μg-50 μg, 30 μg-60 μg, 30 μg-70 μg, 30 μg-75 μg which may be administered to the subject a total of one or two times or more.

Aspects of the invention provide methods of producing an mRNA encoding a concatemeric cancer antigen comprising between 1000 and 3000 nucleotides, the method comprising: (a) binding a first polynucleotide comprising an open reading frame encoding the cancer antigen of any one of claim 1-103 and a second polynucleotide comprising a 5′-UTR to a polynucleotide conjugated to a solid support; (b) ligating the 3′-terminus of the second polynucleotide to the 5′-terminus of the first polynucleotide under suitable conditions, wherein the suitable conditions comprise a DNA Ligase, thereby producing a first ligation product; (c) ligating the 5′ terminus of a third polynucleotide comprising a 3′-UTR to the 3′-terminus of the first ligation product under suitable conditions, wherein the suitable conditions comprise an RNA Ligase, thereby producing a second ligation product; and (d) releasing the second ligation product from the solid support, thereby producing an mRNA encoding the concatemeric cancer antigen comprising between 1000 and 3000 nucleotides.

Aspects of the invention provide methods for treating a subject with a personalized mRNA cancer vaccine, comprising identifying a set of neoepitopes to produce a patient specific mutanome, selecting a set of neoepitopes for the vaccine from the mutanome based on MHC binding strength, MHC binding diversity, predicted degree of immunogenicity, low self reactivity, and/or T cell reactivity, preparing the mRNA vaccine to encode the set of neoepitopes, and administering the mRNA vaccine to the subject within two months of isolating the sample from the subject.

Aspects of the invention provide methods of identifying a set of neoepitopes for use in a personalized mRNA cancer vaccine having one or more polynucleotides that encode the set of neoepitopes comprising: (a) identifying a patient specific mutanome by analyzing a patient transcriptome and a patient exome, (b) selecting a subset of 15-500 neoepitopes from the mutanome using a weighted value for the neoepitopes based on at least three of: an assessment of gene or transcript-level expression in patient RNA-seq; variant call confidence score; RNA-seq allele-specific expression; conservative vs. non-conservative amino acid substitution; position of point mutation (Centering Score for increased TCR engagement); position of point mutation (Anchoring Score for differential HLA binding); Selfness: <100% core epitope homology with patient WES data; HLA-A and -B IC50 for 8 mers-1 liners; HLA-DRB1 IC50 for 15 mers-20 mers; promiscuity Score; HLA-C IC50 for 8 mers-11 mers; HLA-DRB3-5 IC50 for 15 mers-20 mers; HLA-DQB1/A1 IC50 for 15 mers-20 mers; HLA-DPB1/A1 IC50 for 15 mers-20 mers; Class I vs Class II proportion; Diversity of patient HLA-A, -B and DRB1 allotypes covered; proportion of point mutation vs complex epitopes; pseudo-epitope HLA binding scores; presence and/or abundance of RNAseq reads, and (c) selecting the set of neoepitopes for use in a personalized mRNA cancer vaccine from the subset based on the highest weighted value, wherein the set of neoepitopes comprise 15-40 neoepitopes.

Aspects of the invention provide methods of identifying a set of neoepitopes for use in a personalized mRNA cancer vaccine having one or more polynucleotides that encode the set of neoepitopes comprising: (a) generating a RNA-seq sample from a patient tumor to produce a set of RNA-seq reads, (b) compiling overall counts of nucleotide sequences from all RNA-seq reads, (c) comparing sequence information between the tumor sample and a corresponding database of normal tissues of the same tissue type, and (d) selecting a set of neoepitopes for use in a personalized mRNA cancer vaccine from the subset based on the highest weighted value, wherein the set of neoepitopes comprise 15-40 neoepitopes.

The details of various embodiments of the invention are set forth in the description below. Other features, objects, and advantages of the invention will be apparent from the description and the drawings, and from the claims.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIG. 1 shows confirmation of full read through of the concatamer (SIINFEKL is SEQ ID NO: 231).

FIG. 2 shows antigen-specific responses to Class I epitopes found in both constructs.

FIG. 3 shows antigen-specific responses to Class I epitopes found exclusively in 52 mer constructs.

FIG. 4 shows antigen-specific responses to Class II epitopes found in both constructs (left) and found exclusively in the 52 mer constructs (right).

FIG. 5 is a block diagram of an exemplary computer system on which some embodiments may be implemented.

FIG. 6 shows antigen-specific responses from mice immunized with mRNA encoding a concatemer of 52 murine epitopes (adding epitopes_4a_DX_RX_perm) in combination with a STING immunopotentiator mRNA at varying antigen and STING dosages and antigen:STING ratios. Data shown is for in vitro restimulation with the peptide sequence corresponding to the Class II epitope RNA 2, encoded within the concatemer.

FIG. 7 shows antigen-specific responses from mice immunized with mRNA encoding a concatemer of 52 murine epitopes (adding epitopes_4a_DX_RX_perm) in combination with a STING immunopotentiator mRNA at varying antigen and STING dosages and antigen:STING ratios. Data shown is for in vitro restimulation with the peptide sequence corresponding to the Class II epitope RNA 3, encoded within the concatemer.

FIG. 8 shows antigen-specific responses from mice immunized with mRNA encoding a concatemer of 52 murine epitopes (adding epitopes_4a_DX_RX_perm) in combination with a STING immunopotentiator mRNA at varying antigen and STING dosages and antigen:STING ratios. Data shown is for in vitro restimulation with the peptide sequence corresponding to Class I epitope RNA 7, encoded within the concatemer.

FIG. 9 shows antigen-specific responses from mice immunized with mRNA encoding a concatemer of 52 murine epitopes (adding epitopes_4a_DX_RX_perm) in combination with a STING immunopotentiator mRNA at varying antigen and STING dosages and antigen:STING ratios. Data shown is for in vitro restimulation with the peptide sequence corresponding to Class I epitope RNA 13, encoded within the concatemer.

FIG. 10 shows antigen-specific responses from mice immunized with mRNA encoding a concatemer of 52 murine epitopes (adding epitopes_4a_DX_RX_perm) in combination with a STING immunopotentiator mRNA at varying antigen and STING dosages and antigen:STING ratios. Data shown is for in vitro restimulation with the peptide sequence corresponding to Class I epitope RNA 22, encoded within the concatemer.

FIG. 11 shows antigen-specific responses from mice immunized with mRNA encoding a concatemer of 52 murine epitopes (adding epitopes_4a_DX_RX_perm) in combination with a STING immunopotentiator mRNA at varying antigen and STING dosages and antigen:STING ratios. Data shown is for in vitro restimulation with the peptide sequence corresponding to Class II epitope RNA 10, encoded within the concatemer.

FIG. 12 is a bar graph showing antigen-specific IFN-γ T responses from mice immunized with mRNA encoding a concatemer of 20 murine epitopes (RNA 31) in combination with a STING immunopotentiator mRNA, as compared to standard adjuvants, or unformulated (not encapsulated in LNP). Data shown is for in vitro peptide restimulation with Class II epitopes (RNA 2 and RNA 3) encoded within the concatemer.

FIG. 13 is a bar graph showing antigen-specific IFN-γ T responses from mice immunized with mRNA encoding a concatemer of 20 murine epitopes (RNA 31) in combination with a STING immunopotentiator mRNA, as compared to standard adjuvants, or unformulated (not encapsulated in LNP). Data shown is for in vitro peptide restimulation with Class I epitopes (RNA 7, RNA 10, and RNA 13) encoded within the concatemer.

FIG. 14 is a bar graph showing antigen-specific IFN-γ T responses from mice immunized with mRNA encoding a concatemer of 20 murine epitopes (RNA 31) in combination with a STING immunopotentiator mRNA, wherein the STING construct was administered either simultaneously with the vaccine, 24 hours later or 48 hours later. Data shown is for in vitro peptide restimulation with either Class II epitopes (RNA 2 and RNA 3) or Class I epitopes (RNA 7, RNA 10, RNA 13) encoded within the concatemer.

FIG. 15 depicts KRAS mutations in colorectal cancer as identified in COSMIC, 2012 data set.

FIG. 16 depicts isoform-specific point mutation specificity for HRAS. Data representing total number of tumors with each point mutation were collated from COSMIC v52 release. Single base mutations generating each amino acid substitution are indicated. The most frequent mutations for each isoform for each cancer type are highlighted with grey shading. H/L: hematopoietic/lymphoid tissues. (Prior et al. Cancer Res. 2012 May 15; 72(10): 2457-2467).

FIG. 17 depicts isoform-specific point mutation specificity for KRAS. Data representing total number of tumors with each point mutation were collated from COSMIC v52 release. Single base mutations generating each amino acid substitution are indicated. The most frequent mutations for each isoform for each cancer type are highlighted with grey shading. H/L: hematopoietic/lymphoid tissues. (Prior et al. Cancer Res. 2012 May 15; 72(10): 2457-2467).

FIG. 18 depicts isoform-specific point mutation specificity for NRAS. Data representing total number of tumors with each point mutation were collated from COSMIC v52 release. Single base mutations generating each amino acid substitution are indicated. The most frequent mutations for each isoform for each cancer type are highlighted with grey shading. H/L: hematopoietic/lymphoid tissues. (Prior et al. Cancer Res. 2012 May 15; 72(10): 2457-2467).

FIG. 19 depicts secondary KRAS mutations after acquisition of EGFR blockade resistance. (Diaz et at The molecular evolution of acquired resistance to targeted EGFR blockade in colorectal cancers, Nature 486:537 (2012)).

FIG. 20 depicts secondary KRAS mutations after EGFR blockade. (Misale et al. Emergence of KRAS muations and acquired resistance to anti-EGFR therapy in colorectal cancer, Nature 486:532 (2012)).

FIG. 21 depicts NRAS and KRAS mutation frequency in colorectal cancer as identified using cBioPortal.

### DETAILED DESCRIPTION

Embodiments of the present disclosure provide RNA (e.g., mRNA) vaccines that include a polynucleotide encoding a cancer antigen. Cancer RNA vaccines, as provided herein may be used to induce a balanced immune response, comprising cellular and/or humoral immunity, without many of the risks associated with DNA vaccination. In some embodiments, a vaccine comprises at least one RNA (e.g., mRNA) polynucleotide having an open reading frame encoding a cancer antigen. In some embodiments, a vaccine comprises at least one RNA (e.g., mRNA) polynucleotide having at least one open reading frame encoding a cancer antigen and at least one open reading frame encoding a universal type II T-cell epitope. In another embodiment, a vaccine comprises at least one RNA (e.g., mRNA) polynucleotide having at least one open reading frame encoding a cancer antigen and at least one open reading frame encoding an immune potentiator (e.g., an adjuvant). In some embodiments, a vaccine comprises at least one RNA (e.g., an mRNA) polynucleotide having an open reading frame encoding a cancer antigen (e.g., an activating oncogene mutation peptide).

Although attempts have been made to produce functional RNA vaccines, including mRNA cancer vaccines, the therapeutic efficacy of these RNA vaccines have not yet been fully established. Quite surprisingly, the inventors have discovered a class of formulations for delivering mRNA vaccines that results in significantly enhanced, and in many respects synergistic, immune responses including enhanced T cell responses. The vaccines of the invention include traditional cancer vaccines as well as personalized cancer vaccines. The invention involves, in some aspects, the surprising finding that lipid nanoparticle formulations significantly enhance the effectiveness of mRNA vaccines, including chemically modified and unmodified mRNA vaccines.

The lipid nanoparticle used in the studies described herein has been used previously to deliver siRNA various in animal models as well as in humans. In view of the observations made in association with the siRNA delivery of lipid nanoparticle formulations, the fact that the lipid nanoparticle, in contrast to liposomes, is useful in cancer vaccines is quite surprising. It has been observed that therapeutic delivery of siRNA formulated in lipid nanoparticle causes an undesirable inflammatory response associated with a transient IgM response, typically leading to a reduction in antigen production and a compromised immune response. In contrast to the findings observed with siRNA, the lipid nanoparticle-mRNA cancer vaccine formulations described herein are demonstrated to generate enhanced IgG levels, sufficient for prophylactic and therapeutic methods rather than transient IgM responses. The lipid nanoparticles of the invention are not liposomes. A liposome as used herein is a lipid based structure having a lipid bilayer or monolayer shell with a nucleic acid payload in the core.

The generation of cancer antigens that elicit a desired immune response (e.g. T-cell responses) against targeted polypeptide sequences in vaccine development remains a challenging task. The invention involves technology that overcome hurdles associated with such development. Through the use of the technology of the invention, it is possible to tailor the desired immune response by selecting appropriate T or B cell cancer epitopes and formulating the epitopes or antigens for effective delivery in vivo. Additionally or alternatively, the immune response may be further augmented by selecting one or more universal type II T-cells eptiopes to be delivered in addition to appropriate T and/or B cell cancer epitopes or antigens.

Additionally or alternatively, the mRNA vaccines may include an activating oncogene mutation peptide (e.g., a KRAS mutation peptide). Prior research has shown limited ability to raise T cells specific to the oncogenic mutation. Much of this research was done in the context of the most common HLA allele (A2, which occurs in ˜50% of Caucasians). More recent work has explored the generation of specific T cells against point mutations in the context of less common HLA alleles (A11, C8). These findings have significant implications for the treatment of cancer. Oncogenic mutations are common in many cancers. The ability to target these mutations and generate T cells that are sufficient to kill tumors has broad applicability to cancer therapy. It is quite surprising that delivery of antigens using mRNA would have such a significant advantage over the delivery of peptide vaccines. Thus the invention involves, in some aspects, the surprising finding that activating oncogenic mutation antigens delivered in vivo in the form of an mRNA significantly enhances the effectiveness of cancer therapy.

HLA class I molecules are highly polymorphic trans-membrane glycoproteins composed of two polypeptide chains (heavy chain and light chain). Human leucocyte antigen, the major histocompatibility complex in humans, is specific to each individual and has hereditary features. The class I heavy chains are encoded by three genes: HLA-A, HLA-B and HLA-C. HLA class I molecules are important for establishing an immune response by presenting endogenous antigens to T lymphocytes, which initiates a chain of immune reactions that lead to tumor cell elimination by cytotoxic T cells. Altered levels of production of HLA class I antigens is a widespread phenomenon in malignancies and is accompanied by significant inhibition of anti-tumor T cell function. It represents one of the main mechanisms used by cancer cells to evade immuno-surveillance. Down regulated levels of HLA class I antigens were detected in 90% of NSCLC tumors (n=65). A reduction or loss of HLA was detected in 76% of pancreatic tumor samples (n=19). The expression of HLA class I antigens in colon cancer was dramatically reduced or undetectable in 96% of tumor samples (n=25).

Mounting evidence suggests that two general strategies are utilized by tumor cells to escape immune surveillance: immunoselection (poorly immunogenic tumor cell variants) and immunosubversion (subversion of the immune system). A correlation between changes in HLA class I antigens and the presence of KRAS codon 12 mutations was demonstrated, which suggests a possible inductive effect of KRAS codon 12 mutations on HLA class I antigen regulation in cancer progression. Many frequent cancer mutations are predicted to bind HLA Class I alleles with high-affinity (IC50<=50 nM)7 and may be suitable for prophylactic cancer vaccination.

The therapeutic mRNA can be delivered alone or in combination with other cancer therapeutics such as checkpoint inhibitors to provide a significantly enhanced immune response against tumors. The checkpoint inhibitors can enhance the effects of the mRNA encoding activing oncogenic peptides by eliminating some of the obstacles to promoting an immune response, thus allowing the activated T cells to efficiently promote an immune response against the tumor.

It has been discovered that the mRNA vaccines described herein are superior to current vaccines in several ways. First, the lipid nanoparticle (LNP) delivery is superior to other formulations including liposome or protamine based approaches described in the literature. The use of LNPs enables the effective delivery of chemically modified or unmodified mRNA vaccines. Both modified and unmodified LNP formulated mRNA vaccines are superior to conventional vaccines by a significant degree. In some embodiments the mRNA vaccines of the invention are superior to conventional vaccines by a factor of at least 10 fold, 20 fold, 40 fold, 50 fold, 100 fold, 500 fold or 1,000 fold.

Although attempts have been made to produce functional RNA vaccines, including mRNA vaccines and self-replicating RNA vaccines, the therapeutic efficacy of these RNA vaccines have not yet been fully established. Quite surprisingly, the inventors have discovered, according to aspects of the invention a class of formulations for delivering mRNA vaccines in vivo that results in significantly enhanced, and in many respects synergistic, immune responses including enhanced antigen generation and functional antibody production with neutralization capability. These results can be achieved even when significantly lower doses of the mRNA are administered in comparison with mRNA doses used in other classes of lipid based formulations. The formulations of the invention have demonstrated significant unexpected in vivo immune responses sufficient to establish the efficacy of functional mRNA vaccines as prophylactic and therapeutic agents. Additionally, self-replicating RNA vaccines rely on viral replication pathways to deliver enough RNA to a cell to produce an immunogenic response. The formulations of the invention do not require viral replication to produce enough protein to result in a strong immune response. Thus, the mRNA of the invention are not self-replicating RNA and do not include components necessary for viral replication.

The invention involves, in some aspects, the surprising finding that lipid nanoparticle (LNP) formulations significantly enhance the effectiveness of mRNA vaccines, including chemically modified and unmodified mRNA vaccines. Furthermore, it was found that immunogenicity to epitopes is similar, independent of the total number of epitopes contained within the construct. Epitopes contained in a 52 mer constructs have similar immunogenicity compared to 20mer constructs as measured by epitope-specific IFNγ responses. It was quite unexpected that the increased mRNA length was demonstrated to have no deleterious effect on immunogenicity of epitopes. The last epitope encoded in the 20mer and 52mer (SIINFEKL, SEQ ID NO: 231) was comparable, this also indicates a full read through of the concatamers. Also surprisingly, it was found that antigen-specific responses to Class I epitopes increased when the vaccines were formulated with a constitutively active immune potentiator.

The LNP used in the studies described herein has been used previously to deliver siRNA in various animal models as well as in humans. In view of the observations made in association with the siRNA delivery of LNP formulations, the fact that LNP is useful in vaccines is quite surprising. It has been observed that therapeutic delivery of siRNA formulated in LNP causes an undesirable inflammatory response associated with a transient IgM response, typically leading to a reduction in antigen production and a compromised immune response. In contrast to the findings observed with siRNA, the LNP-mRNA formulations of the invention are demonstrated herein to generate enhanced IgG levels, sufficient for prophylactic and therapeutic methods rather than transient IgM responses.

The mRNA cancer vaccines provide unique therapeutic alternatives to peptide based or DNA vaccines. When the mRNA cancer vaccine is delivered to a cell, the mRNA will be processed into a polypeptide by the intracellular machinery which can then process the polypeptide into immunosensitive fragments capable of stimulating an immune response against the tumor.

In some embodiments, the mRNA cancer vaccine may be administered with an anti-cancer therapeutic agent, including but not limited to, a traditional cancer vaccine. The mRNA cancer vaccine and anti-cancer therapeutic can be combined to enhance immune therapeutic responses even further. The mRNA cancer vaccine and other therapeutic agent may be administered simultaneously or sequentially. When the other therapeutic agents are administered simultaneously they can be administered in the same or separate formulations, but are administered at the same time. The other therapeutic agents are administered sequentially with one another and with the mRNA cancer vaccine, when the administration of the other therapeutic agents and the mRNA cancer vaccine is temporally separated. The separation in time between the administration of these compounds may be a matter of minutes or it may be longer, e.g. hours, days, weeks, months. Other therapeutic agents include but are not limited to anti-cancer therapeutic, adjuvants, cytokines, antibodies, antigens, etc.

The cancer vaccines described herein include at least one ribonucleic acid (RNA) polynucleotide having an open reading frame encoding at least one cancer antigenic polypeptide or an immunogenic fragment thereof (e.g., an immunogenic fragment capable of inducing an immune response to cancer). The antigenic peptide may be a personalized cancer antigen epitope, and/or a recurrent antigen. In some preferred embodiments the vaccine is multiple epitopes of a mixture of each of the above. Thus the cancer vaccines may be traditional or personalized cancer vaccines or mixtures thereof. A traditional cancer vaccine is a vaccine including a cancer antigen that is known to be found in cancers or tumors generally or in a specific type of cancer or tumor. Antigens that are expressed in or by tumor cells are referred to as “tumor associated antigens”. A particular tumor associated antigen may or may not also be expressed in non-cancerous cells. Many tumor mutations are known in the art.

It has been discovered surprisingly that RNA based multiepitopic cancer vaccines, whether formulated as individual epitopes or as a concatemer, can produce optimal immune stimulation through a careful balance of MHC class I epitopes and MHC class II epitopes. RNA vaccines which encode both components have enhanced immunogenicity.

Personalized vaccines, for instance, may include RNA encoding for one or more known cancer antigens specific for the tumor or cancer antigens specific for each subject, referred to as neoepitopes or subject specific epitopes or antigens (referred to as personalized antigens). A “subject specific cancer antigen” is an antigen that has been identified as being expressed in a tumor of a particular patient. The subject specific cancer antigen may or may not be typically present in tumor samples generally. Tumor associated antigens that are not expressed or rarely expressed in non-cancerous cells, or whose expression in non-cancerous cells is sufficiently reduced in comparison to that in cancerous cells and that induce an immune response induced upon vaccination, are referred to as neoepitopes. Neoepitopes, like tumor associated antigens, are completely foreign to the body and thus would not produce an immune response against healthy tissue or be masked by the protective components of the immune system. In some embodiments personalized vaccines based on neoepitopes are desirable because such vaccine formulations will maximize specificity against a patient's specific tumor. Mutation-derived neoepitopes can arise from point mutations, non-synonymous mutations leading to different amino acids in the protein; read-through mutations in which a stop codon is modified or deleted, leading to translation of a longer protein with a novel tumor-specific sequence at the C-terminus; splice site mutations that lead to the inclusion of an intron in the mature mRNA and thus a unique tumor-specific protein sequence; chromosomal rearrangements that give rise to a chimeric protein with tumor-specific sequences at the junction of 2 proteins (i.e., gene fusion); frameshift mutations or deletions that lead to a new open reading frame with a novel tumor-specific protein sequence; and translocations. Thus, in some embodiments the mRNA cancer vaccines include at least 2 cancer antigens including mutations selected from the group consisting of frame-shift mutations and recombinations or any of the other mutations described herein.

Methods for generating personalized cancer vaccines generally involve identification of mutations, e.g., using deep nucleic acid or protein sequencing techniques, identification of neoepitopes, e.g., using application of validated peptide-MHC binding prediction algorithms or other analytical techniques to generate a set of candidate T cell epitopes that may bind to patient HLA alleles and are based on mutations present in tumors, optional demonstration of antigen-specific T cells against selected neoepitopes or demonstration that a candidate neoepitope is bound to HLA proteins on the tumor surface and development of the vaccine. The mRNA cancer vaccines of the invention may include multiple copies of a single neoepitope, multiple different neoepitopes based on a single type of mutation, i.e. point mutation, multiple different neoepitopes based on a variety of mutation types, neoepitopes and other antigens, such as tumor associated antigens or recall antigens.

Examples of techniques for identifying mutations include but are not limited to dynamic allele-specific hybridization (DASH), microplate array diagonal gel electrophoresis (MADGE), pyrosequencing, oligonucleotide-specific ligation, the TaqMan system as well as various DNA “chip” technologies i.e. Affymetrix SNP chips, and methods based on the generation of small signal molecules by invasive cleavage followed by mass spectrometry or immobilized padlock probes and rolling-circle amplification.

The deep nucleic acid or protein sequencing techniques are known in the art. Any type of sequence analysis method can be used. Nucleic acid sequencing may be performed on whole tumor genomes, tumor exomes (protein-encoding DNA), tumor transcriptomes, or exosomes. Real-time single molecule sequencing-by-synthesis technologies rely on the detection of fluorescent nucleotides as they are incorporated into a nascent strand of DNA that is complementary to the template being sequenced. Other rapid high throughput sequencing methods also exist. Protein sequencing may be performed on tumor proteomes. Additionally, protein mass spectrometry may be used to identify or validate the presence of mutated peptides bound to MHC proteins on tumor cells. Peptides can be acid-eluted from tumor cells or from HLA molecules that are immunoprecipitated from tumor, and then identified using mass spectrometry. The results of the sequencing may be compared with known control sets or with sequencing analysis performed on normal tissue of the patient.

Accordingly, the present invention relates to methods for identifying and/or detecting neoepitopes of an antigen. Specifically, the invention provides methods of identifying and/or detecting tumor specific neoepitopes that are useful in inducing a tumor specific immune response in a subject. Optionally, some of these neoepitopes bind to class I HLA proteins with a greater affinity than the wild-type peptide and/or are capable of activating anti-tumor CD8 T-cells. Others bind to class II and activate CD4+ T helper cells. While the important role that class I antigens play in a vaccine have been recognized it has been discovered herein that vaccines composed of a balance of class I and class II antigens actually produce a more robust immune response than a vaccine based on class I or class II alone.

Proteins of MHC class I are present on the surface of almost all cells of the body, including most tumor cells. The proteins of MHC class I are loaded with antigens that usually originate from endogenous proteins or from pathogens present inside cells, and are then presented to cytotoxic T-lymphocytes (CTLs). T-Cell receptors are capable of recognizing and binding peptides complexed with the molecules of MHC class I. Each cytotoxic T-lymphocyte expresses a unique T-cell receptor which is capable of binding specific MHC/peptide complexes.

Using computer algorithms, it is possible to predict potential neoepitopes, i.e. peptide sequences, which are bound by the MHC molecules of class I or class II in the form of a peptide-presenting complex and then, in this form, recognized by the T-cell receptors of T-lymphocytes. Examples of programs useful for identifying peptides which will bind to MHC include for instance: Lonza Epibase, SYFPEITHI (Rammensee et al, Immunogenetics, 50 (1999), 213-219) and HLA_BIND (Parker et al., J. Immunol., 152 (1994), 163-175).

Once putative neoepitopes are selected, they can be further tested using in vitro and/or in vivo assays. Conventional in vitro lab assays, such as Elispot assays may be used with an isolate from each patient, to refine the list of neoepitopes selected based on the algorithm's predictions.

The mRNA cancer vaccines of the invention are compositions, including pharmaceutical compositions. The invention also encompasses methods for the selection, design, preparation, manufacture, formulation, and/or use of mRNA cancer vaccines. Also provided are systems, processes, devices and kits for the selection, design and/or utilization of the mRNA cancer vaccines described herein.

The mRNA vaccines of the invention may include one or more cancer antigens. In some embodiments the mRNA vaccine is composed of 45 or more, 46 or more, 47 or more, 48 or more, 49 or more, 50 or more, 51 or more, 52 or more, 53 or more, 54 or more, or 55 or more antigens. In other embodiments, the mRNA vaccine is composed of 3 or more, 4 or more, 5 or more, 6 or more, 7 or more, 8 or more, or 9 or more antigens. In other embodiments the mRNA vaccine is composed of 1000 or less, 900 or less, 500 or less, 100 or less, 75 or less, 50 or less, 40 or less, 30 or less, 20 or less or 100 or less cancer antigens. In yet other embodiments the mRNA vaccine has 3-100, 5-100, 10-100, 15-100, 20-100, 25-100, 30-100, 35-100, 40-100, 45-100, 50-100, 55-100, 60-100, 65-100, 70-100, 75-100, 80-100, 90-100, 5-50, 10-50, 15-50, 20-50, 25-50, 30-50, 35-50, 40-50, 45-50, 100-150, 100-200, 100-300, 100-400, 100-500, 50-500, 50-800, 50-1,000, or 100-1,000 cancer antigens.

In some embodiments the mRNA cancer vaccines and vaccination methods include epitopes or antigens based on specific mutations (neoepitopes) and those expressed by cancer-germline genes (antigens common to tumors found in multiple patients).

An epitope, also known as an antigenic determinant, as used herein is a portion of an antigen that is recognized by the immune system in the appropriate context, specifically by antibodies, B cells, or T cells. Epitopes include B cell epitopes and T cell epitopes. B-cell epitopes are peptide sequences which are required for recognition by specific antibody producing B-cells. B cell epitopes refer to a specific region of the antigen that is recognized by an antibody. The portion of an antibody that binds to the epitope is called a paratope. An epitope may be a conformational epitope or a linear epitope, based on the structure and interaction with the paratope. A linear, or continuous, epitope is defined by the primary amino acid sequence of a particular region of a protein. The sequences that interact with the antibody are situated next to each other sequentially on the protein, and the epitope can usually be mimicked by a single peptide. Conformational epitopes are epitopes that are defined by the conformational structure of the native protein. These epitopes may be continuous or discontinuous, i.e. components of the epitope can be situated on disparate parts of the protein, which are brought close to each other in the folded native protein structure.

T-cell epitopes are peptide sequences which, in association with proteins on APC, are required for recognition by specific T-cells. T cell epitopes are processed intracellularly and presented on the surface of APCs, where they are bound to MHC molecules including MHC class II and MHC class I. The peptide epitope may be any length that is reasonable for an epitope. In some embodiments the peptide epitope is 9-30 amino acids. In other embodiments the length is 9-22, 9-29, 9-28, 9-27, 9-26, 9-25, 9-24, 9-23, 9-21, 9-20, 9-19, 9-18, 10-22, 10-21, 10-20, 11-22, 22-21, 11-20, 12-22, 12-21, 12-20, 13-22, 13-21, 13-20, 14-19, 15-18, or 16-17 amino acids.

In some embodiments, the peptide epitopes comprise at least one MHC class I epitope and at least one MHC class II epitope. In some embodiments, at least 10% of the epitopes are MHC class I epitopes. In some embodiments, at least 20% of the epitopes are MHC class I epitopes. In some embodiments, at least 30% of the epitopes are MHC class I epitopes. In some embodiments, at least 40% of the epitopes are MHC class I epitopes. In some embodiments, at least 50%, 60%, 70%, 80%, 90% or 100% of the epitopes are MHC class I epitopes. In some embodiments, at least 10% of the epitopes are MHC class II epitopes. In some embodiments, at least 20% of the epitopes are MHC class II epitopes. In some embodiments, at least 30% of the epitopes are MHC class II epitopes. In some embodiments, at least 40% of the epitopes are MHC class II epitopes. In some embodiments, at least 50%, 60%, 70%, 80%, 90% or 100% of the epitopes are MHC class II epitopes. In some embodiments, the ratio of MHC class I epitopes to MHC class II epitopes is a ratio selected from about 10%:about 90%; about 20%:about 80%; about 30%:about 70%; about 40%:about 60%; about 50%:about 50%; about 60%:about 40%; about 70%:about 30%; about 80%:about 20%; about 90%:about 10% MHC class 1:MHC class II epitopes. In one embodiment, the ratio of MHC class I:MHC class II epitopes is 3:1. In some embodiments, the ratio of MHC class II epitopes to MHC class I epitopes is a ratio selected from about 10%:about 90%; about 20%:about 80%; about 30%:about 70%; about 40%:about 60%; about 50%:about 50%; about 60%:about 40%; about 70%:about 30%; about 80%:about 20%; about 90%:about 10% MHC class II:MHC class I epitopes. In one embodiment, the ratio of MHC class II:MHC class I epitopes is 1:3. In some embodiments, at least one of the peptide epitopes of the cancer vaccine is a B cell epitope. In some embodiments, the T cell epitope of the cancer vaccine comprises between 8-11 amino acids. In some embodiments, the B cell epitope of the cancer vaccine comprises between 13-17 amino acids.

In other aspects, the cancer vaccine of the invention comprises an mRNA vaccine encoding multiple peptide epitope antigens, arranged with one or more interspersed universal type II T-cell epitopes. The universal type II T-cell epitopes, include, but are not limited to ILMQYIKANSKFIGI (Tetanus toxin; SEQ ID NO: 226), FNNFTVSFWLRVPKVSASHLE, (Tetanus toxin; SEQ ID NO: 227), QYIKANSKFIGITE (Tetanus toxin; SEQ ID NO: 228) QSIALSSLMVAQAIP (Diptheria toxin; SEQ ID NO: 229), and AKFVAAWTLKAAA (pan-DR epitope (PADRE); SEQ ID NO: 230). In some embodiments, the mRNA vaccine comprises the same universal type II T-cell epitope. In other embodiments, the mRNA vaccine comprises 2, 3, 4, 5, 6, 7, 8, 9, 10, 15, or 20 different universal type II T-cell epitopes. In some embodiments, the one or more universal type II T-cell epitope(s) are interspersed between every cancer antigen. In other embodiments, the one or more universal type II T-cell epitope(s) are interspersed between every 2, 3, 4, 5, 6, 7, 8, 9, 10, 11, 12, 13, 14, 15, 16, 17, 18, 19, 20, 22, 24, 26, 28, 30, 35, 40, 45, 50, 55, 60, 65, 70, 75, 80, 85, 90, or 100 cancer antigens.

The cancer vaccine of the invention, in some aspects comprises an mRNA vaccine encoding multiple peptide epitope antigens arranged with a single nucleotide spacer between the epitopes or directly to one another without a spacer between the epitopes. The multiple epitope antigens includes a mixture of MHC class I epitopes and MHC class II epitopes. For instance, the multiple peptide epitope antigens may be a polypeptide having the structure: (X-G-X)₁₋₁₀(G-Y-G-Y)₁₋₁₀(G-X-G-X)₀₋₁₀(G-Y-G-Y)₀₋₁₀, (X-G)₁₋₁₀(G-Y)₁₋₁₀(G-X)₀₋₁₀(G-Y)₀₋₁₀, (X-G-X-G-X)₁₋₁₀(G-Y-G-Y)₁₋₁₀(X-G-X)₀₋₁₀(G-Y-G-Y)₀₋₁₀, (X-G-X)₁₋₁₀(G-Y-G-Y-G-Y)₁₋₁₀(X-G-X)₀₋₁₀(G-Y-G-Y)₀₋₁₀, (X-G-X-G-X-G-X)₁₋₁₀(G-Y-G-Y)₁₋₁₀(X-G-X)₀₋₁₀(G-Y-G-Y)₀₋₁₀, (X-G-X)₁₋₁₀ (G-Y-G-Y-G-Y-G-Y)₁₋₁₀(X-G-X)₀₋₁₀(G-Y-G-Y)₀₋₁₀, (X)₁₋₁₀(Y)₁₋₁₀(X)₀₋₁₀(Y)₀₋₁₀, (Y)₁₋₁₀(X)₁₋₁₀(Y)₀₋₁₀(X)₀₋₁₀, (XX)₁₋₁₀(Y)₁₋₁₀(X)₀₋₁₀(Y)₀₋₁₀, (YY)₁₋₁₀(XX)₁₋₁₀(Y)₀₋₁₀(X)₀₋₁₀, (X)₁₋₁₀(YY)₁₋₁₀(X)₀₋₁₀(Y)₀₋₁₀, (XXX)₁₋₁₀(YYY)₁₋₁₀(XX)₀₋₁₀(YY)₀₋₁₀, (YYY)₁₋₁₀(XXX)₁₋₁₀(YY)₀₋₁₀(XX)₀₋₁₀, (XY)₁₋₁₀(Y)₀₋₁₀(X)₁₋₁₀(Y)₁₋₁₀, (YX)₁₋₁₀(Y)₀₋₁₀(X)₁₋₁₀(Y)₁₋₁₀, (YX)₁₋₁₀(X)₁₋₁₀(Y)₁₋₁₀(Y)₁₋₁₀, (Y-G-Y)₁₋₁₀(G-X-G-X)₁₋₁₀(G-Y-G-Y)₀₋₁₀(G-X-G-X)₀₋₁₀, (Y-G)₁₋₁₀(G-X)₁₋₁₀(G-Y)₀₋₁₀(G-X)₀₋₁₀, (Y-G-Y-G-Y)₁₋₁₀(G-X-G-X)₁₋₁₀(Y-G-Y)₀₋₁₀(G-X-G-X)₀₋₁₀, (Y-G-Y)₁₋₁₀(G-X-G-X-G-X)₁₋₁₀(Y-G-Y)₀₋₁₀(G-X-G-X)₀₋₁₀, (Y-G-Y-G-Y-G-Y)₁₋₁₀(G-X-G-X)₁₋₁₀(Y-G-Y)₀₋₁₀(G-X-G-X)₀₋₁₀, (Y-G-Y)₁₋₁₀(G-X-G-X-G-X-G-X)₁₋₁₀(Y-G-Y)₀₋₁₀(G-X-G-X)₀₋₁₀, (XY)₁₋₁₀(YX)₁₋₁₀(XY)₀₋₁₀(YX)₀₋₁₀, (YX)₁₋₁₀ (XY)₁₋₁₀(Y)₀₋₁₀(X)₀₋₁₀, (YY)₁₋₁₀(X)₁₋₁₀(Y)₀₋₁₀(X)₀₋₁₀, (XY)₁₋₁₀(XY)₁₋₁₀(X)₀₋₁₀(X)₀₋₁₀, (Y)₁₋₁₀(YX)₁₋₁₀(X)₀₋₁₀(Y)₀₋₁₀, (XYX)₁₋₁₀(YXX)₁₋₁₀(YX)₀₋₁₀(YY)₀₋₁₀, or (YYX)₁₋₁₀(XXY)₁₋₁₀(YX)₀₋₁₀(XY)₀₋₁₀,

X is an MHC class I epitope of 10-40 amino acids in length, Y is an MHC class II epitope of 10-40 amino acids in length, and G is glycine.

The cancer vaccine of the invention, in some aspects, comprises an mRNA vaccine encoding multiple peptide epitope antigens arranged with a centrally located single nucleotide polymorphism (SNP) mutation with flanking amino acids on each side of the SNP mutation. In some embodiments, the number of flanking amino acids on each side of the centrally located SNP mutation is 5, 6, 7, 8, 9, 10, 11, 12, 13, 14, 15, 16, 17, 18, 19, 20, 22, 24, 26, 28, or 30. In one embodiment, an epitope of the cancer vaccine comprises an SNP flanked by two Class I sequences, each sequence comprising seven amino acids. In another embodiment, an epitope of the cancer vaccine comprises a SNP flanked by two Class II sequences, each sequence comprising 10 amino acids. In some embodiments, an epitope may comprise a centrally located SNP and flanks which are both Class I sequences, both Class II sequences, or one Class I and one Class II sequence.

Immune Potentiator mRNAs

One aspect of the disclosure pertains to mRNAs that encode a polypeptide that stimulates or enhances an immune response against one or more of the cancer antigens of interest. Such mRNAs that enhance immune responses to the cancer antigen(s) of interest are referred to herein as immune potentiator mRNA constructs or immune potentiator mRNAs, including chemically modified mRNAs (mmRNAs). An immune potentiator of the disclosure enhances an immune response to an antigen of interest in a subject. The enhanced immune response can be a cellular response, a humoral response or both. As used herein, a “cellular” immune response is intended to encompass immune responses that involve or are mediated by T cells, whereas a “humoral” immune response is intended to encompass immune responses that involve or are mediated by B cells. An immune potentiator may enhance an immune response by, for example,
(i) stimulating Type I interferon pathway signaling;
(ii) stimulating NFkB pathway signaling;
(iii) stimulating an inflammatory response;
(iv) stimulating cytokine production; or
(v) stimulating dendritic cell development, activity or mobilization; and
(vi) a combination of any of (i)-(vi).

As used herein, “stimulating Type I interferon pathway signaling” is intended to encompass activating one or more components of the Type I interferon signaling pathway (e.g., modifying phosphorylation, dimerization or the like of such components to thereby activate the pathway), stimulating transcription from an interferon-sensitive response element (ISRE) and/or stimulating production or secretion of Type I interferon (e.g., IFN-α, IFN-β, IFN-ε, IFN-κ and/or IFN-ω). As used herein, “stimulating NFkB pathway signaling” is intended to encompass activating one or more components of the NFkB signaling pathway (e.g., modifying phosphorylation, dimerization or the like of such components to thereby activate the pathway), stimulating transcription from an NFkB site and/or stimulating production of a gene product whose expression is regulated by NFkB. As used herein, “stimulating an inflammatory response” is intended to encompass stimulating the production of inflammatory cytokines (including but not limited to Type I interferons, IL-6 and/or TNFα). As used herein, “stimulating dendritic cell development, activity or mobilization” is intended to encompass directly or indirectly stimulating dendritic cell maturation, proliferation and/or functional activity.

In some aspects, the disclosure provides an mRNA encoding a polypeptide that stimulates or enhances an immune response in a subject in need thereof (e.g., potentiates an immune response in the subject) by, for example, inducing adaptive immunity (e.g., by stimulating Type I interferon production), stimulating an inflammatory response, stimulating NFkB signaling and/or stimulating dendritic cell (DC) development, activity or mobilization in the subject. In some aspects, administration of an immune potentiator mRNA to a subject in need thereof enhances cellular immunity (e.g., T cell-mediated immunity), humoral immunity (e.g., B cell-mediated immunity) or both cellular and humoral immunity in the subject. In some aspects, administration of an immune potentiator mRNA stimulates cytokine production (e.g., inflammatory cytokine production), stimulates cancer antigen—specific CD8⁺ effector cell responses, stimulates antigen-specific CD4⁺ helper cell responses, increases the effector memory CD62L^{lo} T cell population, stimulates B cell activity or stimulates antigen-specific antibody production, including combinations of the foregoing responses. In some aspects, administration of an immune potentiator mRNA stimulates cytokine production (e.g., inflammatory cytokine production) and stimulates antigen-specific CD8⁺ effector cell responses. In some aspects, administration of an immune potentiator mRNA stimulates cytokine production (e.g., inflammatory cytokine production), and stimulates antigen-specific CD4⁺ helper cell responses. In some aspects, administration of an immune potentiator mRNA stimulates cytokine production (e.g., inflammatory cytokine production), and increases the effector memory CD62L^{lo} T cell population. In some aspects, administration of an immune potentiator mRNA stimulates cytokine production (e.g., inflammatory cytokine production), and stimulates B cell activity or stimulates antigen-specific antibody production.

In one embodiment, an immune potentiator increases cancer antigen-specific CD8⁺ effector cell responses (cellular immunity). For example, an immune potentiator can increase one or more indicators of antigen-specific CD8⁺ effector cell activity, including but not limited to CD8+ T cell proliferation and CD8+ T cell cytokine production. For example, in one embodiment, an immune potentiator increases production of IFN-γ, TNFα and/or IL-2 by antigen-specific CD8+ T cells. In various embodiments, an immune potentiator can increase CD8+ T cell cytokine production (e.g., IFN-γ, TNFα and/or IL-2 production) in response to an antigen (as compared to CD8+ T cell cytokine production in the absence of the immune potentiator) by at least 5% or at least 10% or at least 15% or at least 20% or at least 25% or at least 30% or at least 35% or at least 40% or at least 45% or at least 50%. For example, T cells obtained from a treated subject can be stimulated in vitro with the cancer antigens and CD8+ T cell cytokine production can be assessed in vitro. CD8+ T cell cytokine production can be determined by standard methods known in the art, including but not limited to measurement of secreted levels of cytokine production (e.g., by ELISA or other suitable method known in the art for determining the amount of a cytokine in supernatant) and/or determination of the percentage of CD8+ T cells that are positive for intracellular staining (ICS) for the cytokine. For example, intracellular staining (ICS) of CD8+ T cells for expression of IFN-γ, TNFα and/or IL-2 can be carried out by methods known in the art (see e.g., the Examples). In one embodiment, an immune potentiator increases the percentage of CD8+ T cells that are positive by ICS for one or more cytokines (e.g., IFN-γ, TNFα and/or IL-2) in response to an antigen (as compared to the percentage of CD8+ T cells that are positive by ICS for the cytokine(s) in the absence of the immune potentiator) by at least 5% or at least 10% or at least 15% or at least 20% or at least 25% or at least 30% or at least 35% or at least 40% or at least 45% or at least 50%.

In yet another embodiment, an immune potentiator increases the percentage of CD8+ T cells among the total T cell population (e.g., splenic T cells and/or PBMCs), as compared to the percentage of CD8+ T cells in the absence of the immune potentiator. For example, an immune potentiator can increase the percentage of CD8+ T cells among the total T cell population by at least 5% or at least 10% or at least 15% or at least 20% or at least 25% or at least 30% or at least 35% or at least 40% or at least 45% or at least 50%, as compared to the percentage of CD8+ T cells in the absence of the immune potentiator. The total percentage of CD8+ T cells among the total T cell population can be determined by standard methods known in the art, including but not limited to fluorescent activated cell sorting (FACS) or magnetic activated cell sorting (MACS).

In another embodiment, an immune potentiator increases a tumor-specific immune cell response, as determined by a decrease in tumor volume in vivo in the presence of the immune potentiator as compared to tumor volume in the absence of the immune potentiator. For example, an immune potentiator can decrease tumor volume by at least 5% or at least 10% or at least 15% or at least 20% or at least 25% or at least 30% or at least 35% or at least 40% or at least 45% or at least 50%, as compared to tumor volume in the absence of the immune potentiator. Measurement of tumor volume can be determined by methods well established in the art.

In another embodiment, an immune potentiator increases B cell activity (humoral immune response), for example by increasing the amount of antigen-specific antibody production, as compared to antigen-specific antibody production in the absence of the immune potentiator. For example, an immune potentiator can increase antigen-specific antibody production by at least 5% or at least 10% or at least 15% or at least 20% or at least 25% or at least 30% or at least 35% or at least 40% or at least 45% or at least 50%, as compared to antigen-specific antibody production in the absence of the immune potentiator. In one embodiment, antigen-specific IgG production is evaluated. Antigen-specific antibody production can be evaluated by methods well established in the art, including but not limited to ELISA, RIA and the like that measure the level of antigen-specific antibody (e.g., IgG) in a sample (e.g., a serum sample).

In another embodiment, an immune potentiator increases the effector memory CD62L^{lo} T cell population. For example, an immune potentiator can increase the total % of CD62L^{lo} T cells among CD8+ T cells. Among other functions, the effector memory CD62L^{lo} T cell population has been shown to have an important function in lymphocyte trafficking (see e.g., Schenkel, J. M. and Masopust, D. (2014) Immunity 41:886-897). In various embodiments, an immune potentiator can increase the total percentage of effector memory CD62L^{lo} T cells among the CD8+ T cells in response to an antigen (as compared to the total percentage of CD62L^{lo} T cells among the CD8+ T cells population in the absence of the immune potentiator) by at least 5% or at least 10% or at least 15% or at least 20% or at least 25% or at least 30% or at least 35% or at least 40% or at least 45% or at least 50%. The total percentage of effector memory CD62L^{lo} T cells among the CD8+ T cells can be determined by standard methods known in the art, including but not limited to fluorescent activated cell sorting (FACS) or magnetic activated cell sorting (MACS).

The ability of an immune potentiator mRNA construct to enhance an immune response to a cancer antigen can be evaluated in mouse model systems known in the art. In one embodiment, an immune competent mouse model system is used. In one embodiment, the mouse model system comprises C57/B16 mice (e.g., to evaluate antigen-specific CD8+ T cell responses to a cancer antigen, such as described in the Examples). In another embodiment, the mouse model system comprises BalbC mice or CD1 mice (e.g., to evaluate B cell responses, such an antigen-specific antibody responses).

In one embodiment, an immune potentiator polypeptide of the disclosure functions downstream of at least one Toll-like receptor (TLR) to thereby enhance an immune response. Accordingly, in one embodiment, the immune potentiator is not a TLR but is a molecule within a TLR signaling pathway downstream from the receptor itself.

In one embodiment, an mRNA of the disclosure encoding an immune potentiator can comprises one or more modified nucleobases. Suitable modifications are discussed further below.

In one embodiment, an mRNA of the disclosure encoding an immune potentiator is formulated into a lipid nanoparticle. In one embodiment, the lipid nanoparticle further comprises an mRNA encoding a cancer antigen. In one embodiment, the lipid nanoparticle is administered to a subject to enhance an immune response against the cancer antigen in the subject. Suitable nanoparticles and methods of use are discussed further below.

Immune Potentiator mRNAs that Stimulate Type I Interferon

In some aspects, the disclosure provides an immune potentiator mRNA encoding a polypeptide that stimulates or enhances an immune response against an antigen of interest by simulating or enhancing Type I interferon pathway signaling, thereby stimulating or enhancing Type I interferon (IFN) production. It has been established that successful induction of anti-tumor or anti-microbial adaptive immunity requires Type I IFN signaling (see e.g., Fuertes, M. B. et at (2013) Trends Immunol. 34:67-73). The production of Type I IFNs (including IFN-α, IFN-β, IFN-ε, IFN-κ and IFN-ω) plays a role in clearance of microbial infections, such as viral infections. It has also been appreciated that host cell DNA (for example derived from damaged or dying cells) is capable of inducing Type I interferon production and that the Type I IFN signaling pathway plays a role in the development of adaptive anti-tumor immunity. However, many pathogens and cancer cells have evolved mechanisms to reduce or inhibit Type I interferon responses. Thus, activation (including stimulation and/or enhancement) of the Type I IFN signaling pathway in a subject in need thereof, by providing an immune potentiator mRNA of the disclosure to the subject, stimulates or enhances an immune response in the subject in a wide variety of clinical situations, including treatment of cancer and pathogenic infections, as well as in potentiating vaccine responses to provide protective immunity.

Type I interferons (IFNs) are pro-inflammatory cytokines that are rapidly produced in multiple different cell types, typically upon viral infection, and are known to have a wide variety of effects. The canonical consequences of type I IFN production in vivo is the activation of antimicrobial cellular programs and the development of innate and adaptive immune responses. Type I IFN induces a cell-intrinsic antimicrobial state in infected and neighboring cells that limits the spread of infectious agents, particularly viral pathogens. Type I IFN also modulates innate immune cell activation (e.g., maturation of dendritic cells) to promote antigen presentation and nature killer cell functions. Type I IFN also promotes the development of high-affinity antigen-specific T and B cell responses and immunological memory (Ivashkiv and Donlin (2014) Nat Rev Immunol 14(1):36-49).

Type I IFN activates dendritic cells (DCs) and promotes their T cell stimulatory capacity through autocrine signaling (Montoya et al., (2002) Blood 99:3263-3271). Type I IFN exposure facilitates maturation of DCs via increasing the expression of chemokine receptors and adhesion molecules (e.g., to promote DC migration into draining lymph nodes), co-stimulatory molecules, and MHC class I and class II antigen presentation. DCs that mature following type I IFN exposure can effectively prime protective T cell responses (Wijesundara et al., (2014) Front Immunol 29(412) and references therein).

Type I IFN can either promote or inhibit T cell activation, proliferation, differentiation and survival depending largely on the timing of type I IFN signaling relative to T cell receptor signaling (Crouse et al., (2015) Nat Rev Immunol 15:231-242). Early studies revealed that MHC-I expression is upregulated in response to type I IFN in multiple cell types (Lindahl et al., (1976), J Infect Dis 133(Suppl):A66-A68; Lindahl et al., (1976) Proc NatlAcadSci USA 17:1284-1287) which is a requirement for optimal T cell stimulation, differentiation, expansion and cytolytic activity. Type I IFN can exert potent co-stimulatory effects on CD8 T cells, enhancing CD8 T cell proliferation and differentiation (Curtsinger et al., (2005) J Immunol 174:4465-4469; Kolumam et al., (2005) J Exp Med 202:637-650).

Similar to effects on T cells, type I IFN signaling has both positive and negative effects on B cell responses depending on the timing and context of exposure (Braun et at, (2002) Int Immunol 14(4):411-419; Lin et al, (1998) 187(1):79-87). The survival and maturation of immature B cells can be inhibited by type I IFN signaling. In contrast to immature B cells, type I IFN exposure has been shown to promote B cell activation, antibody production and isotype switch following viral infection or following experimental immunization (Le Bon et al, (2006) J Immunol 176:4:2074-2078; Swanson et al., (2010) J Exp Med 207:1485-1500).

A number of components involved in Type I IFN pathway signaling have been established, including STING, Interferon Regulatory Factors, such as IRF1, IRF3, IRF5, IRF7, IRF8, and IRF9, TBK1, IKKi, MyD88 and TRAM. Additional components involved in Type I IFN pathway signaling include TRAF3, TRAF6, IRAK-1, IRAK-4, TRIF, IPS-1, TLR-3, TLR-4, TLR-7, TLR-8, TLR-9, RIG-1, DAI, and IFI16.

Accordingly, in one embodiment, an immune potentiator mRNA encodes any of the foregoing components involved in Type I IFN pathway signaling.

Immune Potentiator mRNA Encoding STING

The present disclosure encompasses mRNA (including mmRNA) encoding STING, including constitutively active forms of STING, as immune potentiators. STING (STimulator of INterferon Genes; also known as transmembrane protein 173 (TMEM173), mediator of IRF3 activation (MITA), methionine-proline-tyrosine-serine (MPYS), and ER IFN stimulator (ERIS)) is a 379 amino acid, endoplasmic reticulum (ER) resident transmembrane protein that functions as a signaling molecule controlling the transcription of immune response genes, including type I IFNs and pro-inflammatory cytokines (Ishikawa & Barber, (2008) Nature 455:647-678; Ishikawa et al., (2009) Nature 461:788-792; Barber (2010) Nat Rev Immunol 15(12):760-770).

STING functions as a signaling adaptor linking the cytosolic detection of DNA to the TBK1/IRF3/Type I IFN signaling axis. The signaling adaptor functions of STING are activated through the direct sensing of cyclic dinucleotides (CDNs). Examples of CDNs include cyclic di-GMP (guanosine 5′-monophosphate), cyclic di-AMP (adenosine 5′-monophosphate) and cyclic GMP-AMP (cGAMP). Initially characterized as ubiquitous bacterial secondary messengers, CDNs are now known to constitute a class of pathogen-associated molecular pattern molecules (PAMPs) that activate the TBK1/IRF3/type I IFN signaling axis via direct interaction with STING. STING is capable of sensing aberrant DNA species and/or CDNs in the cytosol of the cell, including CDNs derived from bacteria, and/or from the host protein cyclic GMP-AMP synthase (cGAS). The cGAS protein is a DNA sensor that produces cGAMP in response to detection of DNA in the cytosol (Burdette et at, (2011) Nature 478:515-518; Sun et al, (2013) Science 339:786-791; Diner et a, (2013) Cell Rep 3:1355-1361; Ablasser et al., (2013) Nature 498:380-384).

Upon binding to a CDN, STING dimerizes and undergoes a conformational change that promotes formation of a complex with TANK-binding kinase 1 (TBK1) (Ouyang et al., (2012) Immunity 36(6): 1073-1086). This complex translocates to the perinuclear Golgi, resulting in delivery of TBK1 to endolysosomal compartments where it phosphorylates IRF3 and NF-κB transcription factors (Zhong et al., (2008) Immunity 29:538-550). A recent study has shown that STING functions as a scaffold by binding to both TBK1 and IRF3 to specifically promote the phosphorylation of IRF3 by TBK1 (Tanaka & Chen, (2012) Sci Signal 5(214):ra20). Activation of the IRF3-, IRF7- and NF-κB-dependent signaling pathways induces the production of cytokines and other immune response-related proteins, such as type I IFNs, which promote anti-pathogen and/or anti-tumor activity.

A number of studies have investigated the use of CDN agonists of STING as potential vaccine adjuvants or immunomodulatory agents to elicit humoral and cellular immune responses (Dubensky et al, (2013) Ther Adv Vaccines 1(4):131-143 and references therein). Initial studies demonstrated that administration of the CDN c-di-GMP attenuated Staphylococcus aureus infection in vivo, reducing the number of recovered bacterial cells in a mouse infection model yet c-di-GMP had no observable inhibitory or bactericidal effect on bacterial cells in vitro suggesting the reduction in bacterial cells was due to an effect on the host immune system (Karaolis et al, (2005) Antimicrob Agents Chemother 49:1029-1038; Karaolis et al., (2007) Infect Immun 75:4942-4950). Recent studies have shown that synthetic CDN derivative molecules formulated with granulocyte-macrophage colony-stimulating factor (GM-CSF)-producing cancer vaccines (termed STINGVAX) elicit enhanced in vivo antitumor effects in therapeutic animal models of cancer as compared to immunization with GM-CSF vaccine alone (Fu et al., (2015) Sci Transl Med 7(283):283ra52), suggesting that CDN are potent vaccine adjuvants.

Mutant STING proteins resulting from polymorphisms mapped to the human TMEM173 gene have been described exhibiting a gain-of function or constitutively active phenotype. When expressed in vitro, mutant STING alleles were shown to potently stimulate induction of type I IFN (Liu et al., (2014) N Engl J Med 371:507-518; Jeremiah et al., (2014) J Clin Invest 124:5516-5520; Dobbs et al., (2015) Cell Host Microbe 18(2):157-168; Tang & Wang, (2015) PLoS ONE 10(3):e0120090; Melki et al., (2017) J Allergy Clin Immunol In Press; Konig et al, (2017) Ann Rheum Dis 76(2):468-472; Burdette et al (2011) Nature 478:515-518).

Provided herein are modified mRNAs (mmRNAs) encoding constitutively active forms of STING, including mutant human STING isoforms for use as immune potentiators as described herein. mmRNAs encoding constitutively active forms of STING, including mutant human STING isoforms are set forth in the Sequence Listing herein. The amino acid residue numbering for mutant human STING polypeptides used herein corresponds to that used for the 379 amino acid residue wild type human STING (isoform 1) available in the art as Genbank Accession Number NP_938023.

Accordingly, in one aspect, the disclosure provides a mmRNA encoding a mutant human STING protein having a mutation at amino acid residue 155, in particular an amino acid substitution, such as a V155M mutation. In one embodiment, the mmRNA encodes an amino acid sequence as set forth in SEQ ID NO:1. In one embodiment, the STING V155M mutant is encoded by a nucleotide sequence shown in SEQ ID NO: 199. In one embodiment, the mmRNA comprises a 3′ UTR sequence as shown in SEQ ID NO: 209, which includes an miR122 binding site.

In other aspects, the disclosure provides a mmRNA encoding a mutant human STING protein having a mutation at amino acid residue 284, such as an amino acid substitution. Non-limiting examples of residue 284 substitutions include R284T, R284M and R284K. In certain embodiments, the mutant human STING protein has as a R284T mutation, for example has the amino acid sequence set forth in SEQ ID NO: 2 or is encoded by an the nucleotide sequence shown in SEQ ID NO 200. In certain embodiments, the mutant human STING protein has a R284M mutation, for example has the amino acid sequence as set forth in SEQ ID NO: 3 or is encoded by the nucleotide sequence shown in SEQ ID NO: 201. In certain embodiments, the mutant human STING protein has a R284K mutation, for example has the amino acid sequence as set forth in SEQ ID NO: 4 or 224, or is encoded by the nucleotide sequence shown in SEQ ID NO: 202 or 225.

In other aspects, the disclosure provides a mmRNA encoding a mutant human STING protein having a mutation at amino acid residue 154, such as an amino acid substitution, such as a N154S mutation. In certain embodiments, the mutant human STING protein has a N154S mutation, for example has the amino acid sequence as set forth in SEQ ID NO: 5 or is encoded by the nucleotide sequence shown in SEQ ID NO: 203.

In yet other aspects, the disclosure provides a mmRNA encoding a mutant human STING protein having a mutation at amino acid residue 147, such as an amino acid substitution, such as a V147L mutation. In certain embodiments, the mutant human STING protein having a V147L mutation has the amino acid sequence as set forth in SEQ ID NO: 6 or is encoded by the nucleotide sequence shown in SEQ ID NO: 204.

In other aspects, the disclosure provides a mmRNA encoding a mutant human STING protein having a mutation at amino acid residue 315, such as an amino acid substitution, such as a E315Q mutation. In certain embodiments, the mutant human STING protein having a E315Q mutation has the amino acid sequence as set forth in SEQ ID NO: 7 or is encoded by the nucleotide sequence shown in SEQ ID NO: 205.

In other aspects, the disclosure provides a mmRNA encoding a mutant human STING protein having a mutation at amino acid residue 375, such as an amino acid substitution, such as a R375A mutation. In certain embodiments, the mutant human STING protein having a R375A mutation has the amino acid sequence as set forth in SEQ ID NO: 8 or is encoded by the nucleotide sequence shown in SEQ ID NO: 206.

In other aspects, the disclosure provides a mmRNA encoding a mutant human STING protein having a one or more or a combination of two, three, four or more of the foregoing mutations. Accordingly, in one aspect the disclosure provides a mmRNA encoding a mutant human STING protein having one or more mutations selected from the group consisting of: V147L, N154S, V155M, R284T, R284M, R284K, E315Q and R375A, and combinations thereof. In other aspects, the disclosure provides a mmRNA encoding a mutant human STING protein having a combination of mutations selected from the group consisting of: V155M and R284T; V155M and R284M; V155M and R284K; V155M and V147L; V155M and N154S; V155M and E315Q; and V155M and R375A.

In other aspects, the disclosure provides a mmRNA encoding a mutant human STING protein having a V155M and one, two, three or more of the following mutations: R284T; R284M; R284K; V147L; N154S; E315Q; and R375A. In other aspects, the disclosure provides a mmRNA encoding a mutant human STING protein having V155M, V147L and N154S mutations. In other aspects, the disclosure provides a mmRNA encoding a mutant human STING protein having V155M, V147L, N154S mutations, and, optionally, a mutation at amino acid 284. In yet other aspects, the disclosure provides a mmRNA encoding a mutant human STING protein having V155M, V147L, N154S mutations, and a mutation at amino acid 284 selected from R284T, R284M and R284K. In other aspects, the disclosure provides a mmRNA encoding a mutant human STING protein having V155M, V147L, N154S, and R284T mutations. In other aspects, the disclosure provides a mmRNA encoding a mutant human STING protein having V155M, V147L, N154S, and R284M mutations. In other aspects, the disclosure provides a mmRNA encoding a mutant human STING protein having V155M, V147L, N154S, and R284K mutations.

In other embodiments, the disclosure provides a mmRNA encoding a mutant human STING protein having a combination of mutations at amino acid residue 147, 154, 155 and, optionally, 284, in particular amino acid substitutions, such as a V147L, N154S, V155M and, optionally, R284M. In certain embodiments, the mutant human STING protein has V147N, N154S and V155M mutations, such as the amino acid sequence as set forth in SEQ ID NO: 9 or encoded by the nucleotide sequence shown in SEQ ID NO: 207. In certain embodiments, the mutant human STING protein has R284M, V147N, N154S and V155M mutations, such as the amino acid sequence as set forth in SEQ ID NO: 10 or encoded by the nucleotide sequence shown in SEQ ID NO: 208.

In another embodiment, the disclosure provides a mmRNA encoding a mutant human STING protein that is a constitutively active truncated form of the full-length 379 amino acid wild type protein, such as a constitutively active human STING polypeptide consisting of amino acids 137-379.

### Agents for Promotion of Antigen Presenting Cells

In some embodiments the RNA vaccines can be combined with agents for promoting the production of antigen presenting cells (APCs), for instance, by converting non-APCs into pseudo-APCs. Antigen presentation is a key step in the initiation, amplification and duration of an immune response. In this process fragments of antigens are presented through the Maj or Histocompatibility Complex (MHC) or Human Leukocyte Antigens (HLA) to T cells driving an antigen-specific immune response. For immune prophylaxis and therapy, enhancing this response is important for improved efficacy. The RNA vaccines of the invention may be designed or enhanced to drive efficient antigen presentation. One method for enhancing APC processing and presentation, is to provide better targeting of the RNA vaccines to antigen presenting cells (APC). Another approach involves activating the APC cells with immune-stimulatory formulations and/or components.

Alternatively, methods for reprograming non-APC into becoming APC may be used with the RNA vaccines of the invention. Importantly, most cells that take up mRNA formulations and are targets of their therapeutic actions are not APC. Therefore, designing a way to convert these cells into APC would be beneficial for efficacy. Methods and approaches for delivering RNA vaccines, e.g., mRNA vaccines to cells while also promoting the shift of a non-APC to an APC are provided herein. In some embodiments a mRNA encoding an APC reprograming molecule is included in the RNA vaccine or coadministered with the RNA vaccine.

An APC reprograming molecule, as used herein, is a molecule that promotes a transition in a non APC cell to an APC-like phenotype. An APC-like phenotype is property that enables MHC class II processing. Thus, an APC cell having an APC-like phenotype is a cell having one or more exogenous molecules (APC reprograming molecule) which has enhanced MHC class II processing capabilities in comparison to the same cell not having the one or more exogenous molecules. In some embodiments an APC reprograming molecule is a CIITA (a central regulator of MHC Class II expression); a chaperone protein such as CLIP, HLA-DO, HLA-DM etc. (enhancers of loading of antigen fragments into MHC Class II) and/or a costimulatory molecule like CD40, CD80, CD86 etc. (enhancers of T cell antigen recognition and T cell activation).

A CIITA protein is a transactivator that enhances activation of transcription of MHC Class II genes (Steimle et al, 1993, Cell 75:135-146) by interacting with a conserved set of DNA binding proteins that associate with the class II promoter region. The transcriptional activation function of CIITA has been mapped to an amino terminal acidic domain (amino acids 26-137). A nucleic acid molecule encoding a protein that interacts with CIITA, termed CIITA-interacting protein 104 (also referred to herein as CIP104). Both CITTA and CIP104 have been shown to enhance transcription from MHC class II promoters and thus are useful as APC reprograming molecule of the invention. In some embodiments the APC reprograming molecule are full length CIITA, CIP104 or other related molecules or active fragments thereof, such as amino acids 26-137 of CIITA, or amino acids having at least 80% sequence identity thereto and maintaining the ability to enhance activation of transcription of MHC Class II genes.

In preferred embodiments the APC reprograming molecule is delivered to a subject in the form of an mRNA encoding the APC reprograming molecule. As such the RNA vaccines of the invention may include an mRNA encoding an APC reprograming molecule. In some embodiments the mRNA in monocistronic. In other embodiments it is polycistronic. In some embodiments the mRNA encoding the one or more antigens is in a separate formulation from the mRNA encoding the APC reprograming molecule. In other embodiments the mRNA encoding the one or more antigens is in the same formulation as the mRNA encoding the APC reprograming molecule. In some embodiments the mRNA encoding the one or more antigens is administered to a subject at the same time as the mRNA encoding the APC reprograming molecule. In other embodiments the mRNA encoding the one or more antigens is administered to a subject at a different time than the mRNA encoding the APC reprograming molecule. For instance, the mRNA encoding the APC reprograming molecule may be administered prior to the mRNA encoding the one or more antigens. The mRNA encoding the APC reprograming molecule may be administered immediately prior to, at least 1 hour prior to, at least 1 day prior to, at least one week prior to, or at least one month prior to the mRNA encoding the antigens.

Alternatively, the mRNA encoding the APC reprograming molecule may be administered after the mRNA encoding the one or more antigens. The mRNA encoding the APC reprograming molecule may be administered immediately after, at least 1 hour after, at least 1 day after, at least one week after, or at least one month after the mRNA encoding the antigens. In some embodiments the antigen is a cancer antigen, such as a patient specific antigen. In other embodiments the antigen is an infectious disease antigen.

In some embodiments the mRNA vaccine may include a recall antigen, also sometimes referred to as a memory antigen. A recall antigen is an antigen that has previously been encountered by an individual and for which there are pre-existent memory lymphocytes. In some embodiments the recall antigen may be an infectious disease antigen that the individual has likely encountered such as an influenza antigen. The recall antigen helps promote a more robust immune response.

The antigens or neoepitopes selected for inclusion in the mRNA vaccine typically will be high affinity binding peptides. In some aspects the antigens or neoepitopes binds an HLA protein with greater affinity than a wild-type peptide. The antigen or neoepitope has an IC50 of at least less than 5000 nM, at least less than 500 nM, at least less than 250 nM, at least less than 200 nM, at least less than 150 nM, at least less than 100 nM, at least less than 50 nM or less in some embodiments. Typically, peptides with predicted IC50<50 nM, are generally considered medium to high affinity binding peptides and will be selected for testing their affinity empirically using biochemical assays of HLA-binding. The cancer antigens can be personalized cancer antigens. Personalized RNA cancer vaccine, for instance, may include RNA encoding for one or more known cancer antigens specific for the tumor or cancer antigens specific for each subject, referred to as neoepitopes or subject specific epitopes or antigens. A “subject specific cancer antigen” is an antigen that has been identified as being expressed in a tumor of a particular patient. The subject specific cancer antigen may or may not be typically present in tumor samples generally. Tumor associated antigens that are not expressed or rarely expressed in non-cancerous cells, or whose expression in non-cancerous cells is sufficiently reduced in comparison to that in cancerous cells and that induce an immune response induced upon vaccination, are referred to as neoepitopes. Neoepitopes, like tumor associated antigens, are completely foreign to the body and thus would not produce an immune response against healthy tissue or be masked by the protective components of the immune system. In some embodiments personalized RNA cancer vaccines based on neoepitopes are desirable because such vaccine formulations will maximize specificity against a patient's specific tumor. Mutation-derived neoepitopes can arise from point mutations, non-synonymous mutations leading to different amino acids in the protein; read-through mutations in which a stop codon is modified or deleted, leading to translation of a longer protein with a novel tumor-specific sequence at the C-terminus; splice site mutations that lead to the inclusion of an intron in the mature mRNA and thus a unique tumor-specific protein sequence; chromosomal rearrangements that give rise to a chimeric protein with tumor-specific sequences at the junction of 2 proteins (i.e., gene fusion); frameshift mutations or deletions that lead to a new open reading frame with a novel tumor-specific protein sequence; and translocations. Thus, in some embodiments the RNA cancer vaccines include at least 1 cancer antigens including mutations selected from the group consisting of frame-shift mutations and recombinations or any of the other mutations described herein.

Methods for generating personalized RNA cancer vaccines generally involve identification of mutations, e.g., using deep nucleic acid or protein sequencing techniques, identification of neoepitopes, e.g., using application of validated peptide-MHC binding prediction algorithms or other analytical techniques to generate a set of candidate T cell epitopes that may bind to patient HLA alleles and are based on mutations present in tumors, optional demonstration of antigen-specific T cells against selected neoepitopes or demonstration that a candidate neoepitope is bound to HLA proteins on the tumor surface and development of the vaccine. The RNA cancer vaccines of the invention may include multiple copies of a single neoepitope, multiple different neoepitopes based on a single type of mutation, i.e. point mutation, multiple different neoepitopes based on a variety of mutation types, neoepitopes and other antigens, such as tumor associated antigens or recall antigens.

Examples of techniques for identifying mutations include but are not limited to dynamic allele-specific hybridization (DASH), microplate array diagonal gel electrophoresis (MADGE), pyrosequencing, oligonucleotide-specific ligation, the TaqMan system as well as various DNA “chip” technologies i.e. Affymetrix SNP chips, and methods based on the generation of small signal molecules by invasive cleavage followed by mass spectrometry or immobilized padlock probes and rolling-circle amplification.

The deep nucleic acid or protein sequencing techniques are known in the art. Any type of sequence analysis method can be used. Nucleic acid sequencing may be performed on whole tumor genomes, tumor exomes (protein-encoding DNA), tumor transcriptomes, or exosomes. Real-time single molecule sequencing-by-synthesis technologies rely on the detection of fluorescent nucleotides as they are incorporated into a nascent strand of DNA that is complementary to the template being sequenced. Other rapid high throughput sequencing methods also exist. Protein sequencing may be performed on tumor proteomes. Additionally, protein mass spectrometry may be used to identify or validate the presence of mutated peptides bound to MHC proteins on tumor cells. Peptides can be acid-eluted from tumor cells or from HLA molecules that are immunoprecipitated from tumor, and then identified using mass spectrometry. The results of the sequencing may be compared with known control sets or with sequencing analysis performed on normal tissue of the patient.

Accordingly, the present invention relates to methods for identifying and/or detecting neoepitopes of an antigen, such as T-cell epitopes. Specifically, the invention provides methods of identifying and/or detecting tumor specific neoepitopes that are useful in inducing a tumor specific immune response in a subject. Optionally, these neoepitopes bind to class I HLA proteins with a greater affinity than the wild-type peptide and/or are capable of activating anti-tumor CD8 T-cells. Identical mutations in any particular gene are rarely found across tumors.

Proteins of MHC class I are present on the surface of almost all cells of the body, including most tumor cells. The proteins of MHC class I are loaded with antigens that usually originate from endogenous proteins or from pathogens present inside cells, and are then presented to cytotoxic T-lymphocytes (CTLs). T-Cell receptors are capable of recognizing and binding peptides complexed with the molecules of MHC class I. Each cytotoxic T-lymphocyte expresses a unique T-cell receptor which is capable of binding specific MHC/peptide complexes.

Using computer algorithms, it is possible to predict potential neoepitopes such as T-cell epitopes, i.e. peptide sequences, which are bound by the MHC molecules of class I or class II in the form of a peptide-presenting complex and then, in this form, recognized by the T-cell receptors of T-lymphocytes. Examples of programs useful for identifying peptides which will bind to MHC include for instance: Lonza Epibase, SYFPEITHI (Rammensee et al., Immunogenetics, 50 (1999), 213-219) and HLA_BIND (Parker et al., J. Immunol., 152 (1994), 163-175).

Once putative neoepitopes are selected, they can be further tested using in vitro and/or in vivo assays. Conventional in vitro lab assays, such as Elispot assays may be used with an isolate from each patient, to refine the list of neoepitopes selected based on the algorithm's predictions. Neoepitope vaccines, methods of use thereof and methods of preparing are all described in PCT/US2016/044918 which is hereby incorporated by reference in its entirety.

The activating oncogene mutation peptides selected for inclusion in the RNA cancer vaccines typically will be high affinity binding peptides. In some aspect the activating oncogene mutation peptide binds an HLA protein with greater affinity than a wild-type peptide. The activating oncogene mutation peptides have an IC50 of at least less than 5000 nM, at least less than 500 nM, at least less than 250 nM, at least less than 200 nM, at least less than 150 nM, at least less than 100 nM, at least less than 50 nM or less in some embodiments. Typically, peptides with predicted IC50<50 nM, are generally considered medium to high affinity binding peptides and will be selected for testing their affinity empirically using biochemical assays of HLA-binding.

In a personalized cancer vaccine, the subject specific cancer antigens may be identified in a sample of a patient. For instance, the sample may be a tissue sample or a tumor sample. For instance, a sample of one or more tumor cells may be examined for the presence of subject specific cancer antigens. The tumor sample may be examined using whole genome, exome or transcriptome analysis in order to identify the subject specific cancer antigens.

Alternatively the subject specific cancer antigens may be identified in an exosome of the subject. When the antigens for a vaccine are identified in an exosome of the subject, such antigens are said to be representative of exosome antigens of the subject.

Exosomes are small microvesicles shed by cells, typically having a diameter of approximately 30-100 nm. Exosomes are classically formed from the inward invagination and pinching off of the late endosomal membrane, resulting in the formation of a multivesicular body (MVB) laden with small lipid bilayer vesicles, each of which contains a sample of the parent cell's cytoplasm. Fusion of the MVB with the cell membrane results in the release of these exosomes from the cell, and their delivery into the blood, urine, cerebrospinal fluid, or other bodily fluids. Exosomes can be recovered from any of these biological fluids for further analysis.

Nucleic acids within exosomes have a role as biomarkers for tumor antigens. An advantage of analyzing exosomes in order to identify subject specific cancer antigens, is that the method circumvents the need for biopsies. This can be particularly advantageous when the patient needs to have several rounds of therapy including identification of cancer antigens, and vaccination.

A number of methods of isolating exosomes from a biological sample have been described in the art. For example, the following methods can be used: differential centrifugation, low speed centrifugation, anion exchange and/or gel permeation chromatography, sucrose density gradients or organelle electrophoresis, magnetic activated cell sorting (MACS), nanomembrane ultrafiltration concentration, Percoll gradient isolation and using microfluidic devices. Exemplary methods are described in US Patent Publication No. 2014/0212871 for instance.

The term “biological sample” refers to a sample that contains biological materials such as a DNA, a RNA and a protein. In some embodiments, the biological sample may suitably comprise a bodily fluid from a subject. The bodily fluids can be fluids isolated from anywhere in the body of the subject, preferably a peripheral location, including but not limited to, for example, blood, plasma, serum, urine, sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid and combinations thereof.

In some embodiments, the progression of the cancer can be monitored to identify changes in the expressed antigens. Thus, in some embodiments the method also involves at least one month after the administration of a cancer mRNA vaccine, identifying at least 2 cancer antigens from a sample of the subject to produce a second set of cancer antigens, and administering to the subject a mRNA vaccine having an open reading frame encoding the second set of cancer antigens to the subject. The mRNA vaccine having an open reading frame encoding second set of antigens, in some embodiments, is administered to the subject 2 months, 3 months, 4 months, 5 months, 6 months, 8 months, 10 months, or 1 year after the mRNA vaccine having an open reading frame encoding the first set of cancer antigens. In other embodiments the mRNA vaccine having an open reading frame encoding second set of antigens is administered to the subject 1½, 2, 2½, 3, 3½, 4, 4½, or 5 years after the mRNA vaccine having an open reading frame encoding the first set of cancer antigens.

### Hotspot Mutations as Neoantigens

In population analyses of cancer, certain mutations occur in a higher percentage of patients than would be expected by chance. These “recurrent” or “hotspot” mutations have often been shown to have a “driver” role in the tumor, producing some change in the cancer cell function that is important to tumor initiation, maintenance, or metastasis, and is therefore selected for in the evolution of the tumor. In addition to their importance in tumor biology and therapy, recurrent mutations provide the opportunity for precision medicine, in which the patient population is stratified into groups more likely to respond to a particular therapy, including but not limited to targeting the mutated protein itself.

Much effort and research on recurrent mutations has focused on non-synonymous (or “missense”) single nucleotide variants (SNVs), but population analyses have revealed that a variety of more complex (non-SNV) variant classifications, such as synonymous (or “silent”), splice site, multi-nucleotide variants, insertions, and deletions, can also occur at high frequencies.

The p53 gene (official symbol TP53) is mutated more frequently than any other gene in human cancers. Large cohort studies have shown that, for most p53 mutations, the genomic position is unique to one or only a few patients and the mutation cannot be used as recurrent neoantigens for therapeutic vaccines designed for a specific population of patients. Surprisingly, a small subset of p53 loci do, however, exhibit a “hotspot” pattern, in which several positions in the gene are mutated with relatively high frequency. Strikingly, a large portion of these recurrently mutated regions occur near exon-intron boundaries, disrupting the canonical nucleotide sequence motifs recognized by the mRNA splicing machinery. Mutation of a splicing motif can alter the final mRNA sequence even if no change to the local amino acid sequence is predicted (i.e., for synonymous or intronic mutations). Therefore, these mutations are often annotated as “noncoding” by common annotation tools and neglected for further analysis, even though they may alter mRNA splicing in unpredictable ways and exert severe functional impact on the translated protein. If an alternatively spliced isoform produces an in-frame sequence change (i.e., no PTC is produced), it can escape depletion by NMD and be readily expressed, processed, and presented on the cell surface by the HLA system. Further, mutation-derived alternative splicing is usually “cryptic”, i.e., not expressed in normal tissues, and therefore may be recognized by T-cells as non-self neoantigens.

In some aspects, the present invention provides neoantigen peptide sequences resulting from certain recurrent somatic cancer mutations in p53, not limited to missense SNVs and often resulting in alternative splicing, for use as targets for therapeutic vaccination. In some embodiments, the mutation, mRNA splicing events, resulting neoantigen peptides, and/or HLA-restricted epitopes include mutations at the canonical 5′ splice site neighboring codon p.T125, inducing a retained intron having peptide sequence TAKSVTCTVSCPEGLASMRLQCLAVSPCISFVWNFGIPLHPLASCQCFFIVYPLNV (SEQ ID NO: 232) that contains epitopes AVSPCISFVW (SEQ ID NO: 233) (HLA-B*57:01, HLA-B*58:01), HPLASCQCFF (SEQ ID NO: 234) (HLA-B*35:01, HLA-B*53:01), FVWNFGIPL (SEQ ID NO: 235) (HLA-A*02:01, HLA-A*02:06, HLA-B*35:01).

In some embodiments, the mutation, mRNA splicing events, resulting neoantigen peptides, and/or HLA-restricted epitopes include mutations at the canonical 5′ splice site neighboring codon p.331, inducing a retained intron having peptide sequence EYFTLQVLSLGTSYQVESFQSNTQNAVFFLTVLPAIGAFAIRGQ (SEQ ID NO: 236) that contains epitopes LQVLSLGTSY (SEQ ID NO: 237) (HLA-B*15:01), FQSNTQNAVF (SEQ ID NO: 238) (HLA-B*15:01).

In some embodiments, the mutation, mRNA splicing events, resulting neoantigen peptides, and/or HLA-restricted epitopes include mutations at the canonical 3′ splice site neighboring codon p.126, inducing a cryptic alternative exonic 3′ splice site producing the novel spanning peptide sequence AKSVTCTMFCQLAK (SEQ ID NO: 239) that contains epitopes CTMFCQLAK (SEQ ID NO: 240) (HLA-A*11:01), KSVTCTMF (SEQ ID NO: 241) (HLA-B*58:01).

In some embodiments, the mutation, mRNA splicing events, resulting neoantigen peptides, and/or HLA-restricted epitopes include mutations at the canonical 5′ splice site neighboring codon p.224, inducing a cryptic alternative intronic 5′ splice site producing the novel spanning peptide sequence VPYEPPEVWLALTVPPSTAWAA (SEQ ID NO: 242) that contains epitopes VPYEPPEVW (SEQ ID NO: 243) (HLA-B*53:01, HLA-B*5 1:01), LTVPPSTAW (SEQ ID NO: 244) (HLA-B*58:01, HLA-B*57:01) In the foregoing sequences, the transcript codon positions refer to the canonical full-length p53 transcript ENST00000269305 (SEQ ID NO: 245) from the Ensembl v83 human genome annotation.

Mutations are typically obtained from a patient's DNA sequencing data to derive neo-epitopes for prior art peptide vaccines. mRNA expression, however, is a more direct measurement of the global space of possible neo-epitopes. For example, some tumor-specific neo-epitopes may arise from splicing changes, insertions/deletions (InDels) resulting in frameshifts, alternative promoters, or epigenetic modifications that are not easily identified using only the exome sequencing data. There is untapped value in identifying these types of complex mutations for neoantigen vaccines because they will increase the number of epitopes capable of binding a patient's unique HLA allotypes. Moreover, the complex variants will be more immunogenic and likely lead to more effective immune responses against tumors due to their difference from self proteins compared to variants resulting from a single amino acid change.

In some aspects, the invention involves a method for identifying patient specific complex mutations and formulating these mutations into effective personalized mRNA vaccines. The methods involve the use of short read RNA-Seq. A major challenge inherent to using short reads for RNA-seq is the fact that multiple mRNA transcript isoforms can be obtained from the same genomic locus, due to alternative splicing and other mechanisms.

Due to the sequencing reads being much shorter than the full-length mRNA transcript, it becomes difficult to map a set of reads back to the correct corresponding isoform within a known gene annotation model. As a result, complex variants that diverge from the known gene annotations (as are common in cancer) can be difficult to discover by standard approaches. The invention, however, involves the identification of short peptides rather than the exact exon composition of the full-length transcript. The methods for identifying short peptides that will be representative of these complex mutations involves a short k-mer counting approach to neo-epitope prediction of complex variants.

A typical next generation sequencing read is 150 base-pairs, which, if capturing a coding region, can resolve 50 codons, or 41 distinct peptide epitopes of length 9 (27 nucleotides). Therefore, using a simple, computationally scalable operation to count all 27-mers from an RNA-seq sample, the results can be compared versus normal tissue from the same sample, or to a precomputed database of 27-mers from RNA-seq of normal tissues (e.g., GTEx).

An mRNA vaccine containing neo-epitopes predicted from RNA-seq data can be created, whereby 1) all possible 27-mers are counted from all RNA-seq reads from a tumor sample, 2) the open reading frame for each read is predicted by aligning any part of the entire read to the transcriptome, and 3) 27-mer counts are compared to the corresponding 27-mer counts of the matched normal sample and/or a database of normal tissues from the same tissue type, and 4) DNA-seq data from the same tumor is used to add confidence to the neo-epitope predictions, if there is a somatic mutation found in the same gene. Regarding point (4), often a mutation can cause transcriptional or splicing changes that result in a change of the mRNA sequence that is not directly predictable from the mutation itself. For example, a splice site mutation may be predicted to cause exon skipping, but it is not possible to know with certainty which downstream exon will be chosen by the splicing machinery in its place.

In one embodiment, the invention provides an mRNA vaccine comprising a concatemeric polyepitope construct or set of individual epitope constructs containing open reading frame (ORF) coding for neoantigen peptides 1 through 4.

In one embodiment, the invention provides the selective administration of a vaccine containing or coding for peptides 1-4, based on the patient's tumor containing any of the above mutations.

In one embodiment, the invention provides the selective administration of the vaccine based on the dual criteria of the 1) patient's tumor containing any of the above mutations and 2) the patient's normal HLA type containing the corresponding HLA allele predicted to bind to the resulting neoantigen.

It has been discovered that the mRNA vaccines described herein are superior to current vaccines in several ways. First, the lipid nanoparticle (LNP) delivery is superior to other formulations including liposome or protamine based approaches described in the literature and no additional adjuvants are to be necessary. The use of LNPs enables the effective delivery of chemically modified or unmodified mRNA vaccines. Both modified and unmodified LNP formulated mRNA vaccines are superior to conventional vaccines by a significant degree. In some embodiments the mRNA vaccines of the invention are superior to conventional vaccines by a factor of at least 10 fold, 20 fold, 40 fold, 50 fold, 100 fold, 500 fold or 1,000 fold.

Although attempts have been made to produce functional RNA vaccines, including mRNA vaccines and self-replicating RNA vaccines, the therapeutic efficacy of these RNA vaccines have not yet been fully established. Quite surprisingly, the inventors have discovered, according to aspects of the invention a class of formulations for delivering mRNA vaccines in vivo that results in significantly enhanced, and in many respects synergistic, immune responses including enhanced antigen generation and functional antibody production with neutralization capability. These results can be achieved even when significantly lower doses of the mRNA are administered in comparison with mRNA doses used in other classes of lipid based formulations. The formulations of the invention have demonstrated significant unexpected in vivo immune responses sufficient to establish the efficacy of functional mRNA vaccines as prophylactic and therapeutic agents. Additionally, self-replicating RNA vaccines rely on viral replication pathways to deliver enough RNA to a cell to produce an immunogenic response. The formulations of the invention do not require viral replication to produce enough protein to result in a strong immune response. Thus, the mRNA of the invention are not self-replicating RNA and do not include components necessary for viral replication.

The invention involves, in some aspects, the surprising finding that lipid nanoparticle (LNP) formulations significantly enhance the effectiveness of mRNA vaccines, including chemically modified and unmodified mRNA vaccines. The efficacy of mRNA vaccines formulated in LNP was examined in vivo using several distinct tumor antigens. In addition to providing an enhanced immune response, the formulations of the invention generate a more rapid immune response with fewer doses of antigen than other vaccines tested. The mRNA-LNP formulations of the invention also produce quantitatively and qualitatively better immune responses than vaccines formulated in a different carriers. Additionally, the mRNA-LNP formulations of the invention are superior to other vaccines even when the dose of mRNA is lower than other vaccines.

The LNP used in the studies described herein has been used previously to deliver siRNA in various animal models as well as in humans. In view of the observations made in association with the siRNA delivery of LNP formulations, the fact that LNP is useful in vaccines is quite surprising. It has been observed that therapeutic delivery of siRNA formulated in LNP causes an undesirable inflammatory response associated with a transient IgM response, typically leading to a reduction in antigen production and a compromised immune response. In contrast to the findings observed with siRNA, the LNP-mRNA formulations of the invention are demonstrated herein to generate enhanced IgG levels, sufficient for prophylactic and therapeutic methods rather than transient IgM responses.

### Nucleic Acids/Polynucleotides

Cancer vaccines, as provided herein, comprise at least one (one or more) ribonucleic acid (RNA) polynucleotide having an open reading frame encoding at least one cancer antigenic polypeptide. The term “nucleic acid,” in its broadest sense, includes any compound and/or substance that comprises a polymer of nucleotides. These polymers are referred to as polynucleotides.

Nucleic acids (also referred to as polynucleotides) may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

In some embodiments, polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo.

The basic components of an mRNA molecule typically include at least one coding region, a 5′ untranslated region (UTR), a 3′ UTR, a 5′ cap and a poly-A tail. Polynucleotides of the present disclosure may function as mRNA but can be distinguished from wild-type mRNA in their functional and/or structural design features which serve to overcome existing problems of effective polypeptide expression using nucleic-acid based therapeutics.

In some embodiments, a RNA polynucleotide of a cancer vaccine encodes 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9 or 9-10 antigenic polypeptides. In some embodiments, a RNA polynucleotide of a cancer vaccine encodes at least 10, 20, 30, 40, 50, 60, 70, 80, 90 or 100 antigenic polypeptides. In some embodiments, a RNA polynucleotide of a cancer vaccine encodes at least 100 or at least 200 antigenic polypeptides. In some embodiments, a RNA polynucleotide of a cancer vaccine encodes 1-10, 5-15, 10-20, 15-25, 20-30, 25-35, 30-40, 35-45, 40-50, 55-65, 60-70, 65-75, 70-80, 75-85, 80-90, 85-95, 90-100, 1-50, 1-100, 2-50 or 2-100 antigenic polypeptides.

In some embodiments, a RNA polynucleotide of a cancer vaccine encodes 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9 or 9-10 activating oncogene mutation peptides. In some embodiments, a RNA polynucleotide of a cancer vaccine encodes at least 10, 20, 30, 40, 50, 60, 70, 80, 90 or 100 activating oncogene mutation peptides. In some embodiments, a RNA polynucleotide of a cancer vaccine encodes at least 100 or at least 200 activating oncogene mutation peptides. In some embodiments, a RNA polynucleotide of a cancer vaccine encodes 1-10, 5-15, 10-20, 15-25, 20-30, 25-35, 30-40, 35-45, 40-50, 55-65, 60-70, 65-75, 70-80, 75-85, 80-90, 85-95, 90-100, 1-50, 1-100, 2-50 or 2-100 activating oncogene mutation peptides.

Polynucleotides of the present disclosure, in some embodiments, are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence (e.g., a naturally-occurring or wild-type mRNA sequence encoding a polypeptide or protein of interest (e.g., an antigenic protein or polypeptide)). In some embodiments, a codon optimized sequence shares less than 90% sequence identity to a naturally-occurring or wild-type sequence (e.g., a naturally-occurring or wild-type mRNA sequence encoding a polypeptide or protein of interest (e.g., an antigenic protein or polypeptide)). In some embodiments, a codon optimized sequence shares less than 85% sequence identity to a naturally-occurring or wild-type sequence (e.g., a naturally-occurring or wild-type mRNA sequence encoding a polypeptide or protein of interest (e.g., an antigenic protein or polypeptide)). In some embodiments, a codon optimized sequence shares less than 80% sequence identity to a naturally-occurring or wild-type sequence (e.g., a naturally-occurring or wild-type mRNA sequence encoding a polypeptide or protein of interest (e.g., an antigenic protein or polypeptide)). In some embodiments, a codon optimized sequence shares less than 75% sequence identity to a naturally-occurring or wild-type sequence (e.g., a naturally-occurring or wild-type mRNA sequence encoding a polypeptide or protein of interest (e.g., an antigenic protein or polypeptide)).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a polypeptide or protein of interest (e.g., an antigenic protein or polypeptide)). In some embodiments, a codon optimized sequence shares between 65% and 75% or about 80% sequence identity to a naturally-occurring or wild-type sequence (e.g., a naturally-occurring or wild-type mRNA sequence encoding a polypeptide or protein of interest (e.g., an antigenic protein or polypeptide)).

In some embodiments a codon optimized RNA may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Antigens/Antigenic Polypeptides

In some embodiments, a cancer polypeptide (e.g., an activating oncogene mutation peptide) is longer than 5 amino acids and shorter than 50 amino acids. In some embodiments, a cancer polypeptide is longer than 25 amino acids and shorter than 50 amino acids. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. Polypeptides may also comprise single chain or multichain polypeptides such as antibodies or insulin and may be associated or linked. Most commonly, disulfide linkages are found in multichain polypeptides. The term polypeptide may also apply to amino acid polymers in which at least one amino acid residue is an artificial chemical analogue of a corresponding naturally-occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants possess at least 50% identity to a native or reference sequence. In some embodiments, variants share at least 80%, or at least 90% identity with a native or reference sequence.

In some embodiments “variant mimics” are provided. As used herein, the term “variant mimic” is one which contains at least one amino acid that would mimic an activated sequence. For example, glutamate may serve as a mimic for phosphoro-threonine and/or phosphoro-serine. Alternatively, variant mimics may result in deactivation or in an inactivated product containing the mimic, for example, phenylalanine may act as an inactivating substitution for tyrosine; or alanine may act as an inactivating substitution for serine.

“Orthologs” refers to genes in different species that evolved from a common ancestral gene by speciation. Normally, orthologs retain the same function in the course of evolution. Identification of orthologs is critical for reliable prediction of gene function in newly sequenced genomes.

“Analogs” is meant to include polypeptide variants which differ by one or more amino acid alterations, for example, substitutions, additions or deletions of amino acid residues that still maintain one or more of the properties of the parent or starting polypeptide.

The present disclosure provides several types of compositions that are polynucleotide or polypeptide based, including variants and derivatives. These include, for example, substitutional, insertional, deletion and covalent variants and derivatives. The term “derivative” is used synonymously with the term “variant” but generally refers to a molecule that has been modified and/or changed in any way relative to a reference molecule or starting molecule.

As such, polynucleotides encoding peptides or polypeptides containing substitutions, insertions and/or additions, deletions and covalent modifications with respect to reference sequences, in particular the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to polypeptides are those that have at least one amino acid residue in a native or starting sequence removed and a different amino acid inserted in its place at the same position. Substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Features” when referring to polypeptide or polynucleotide are defined as distinct amino acid sequence-based or nucleotide-based components of a molecule respectively. Features of the polypeptides encoded by the polynucleotides include surface manifestations, local conformational shape, folds, loops, half-loops, domains, half-domains, sites, termini or any combination thereof.

As used herein when referring to polypeptides the term “domain” refers to a motif of a polypeptide having one or more identifiable structural or functional characteristics or properties (e.g., binding capacity, serving as a site for protein-protein interactions).

As used herein when referring to polypeptides the terms “site” as it pertains to amino acid based embodiments is used synonymously with “amino acid residue” and “amino acid side chain.” As used herein when referring to polynucleotides the terms “site” as it pertains to nucleotide based embodiments is used synonymously with “nucleotide.” A site represents a position within a peptide or polypeptide or polynucleotide that may be modified, manipulated, altered, derivatized or varied within the polypeptide or polynucleotide based molecules.

As used herein the terms “termini” or “terminus” when referring to polypeptides or polynucleotides refers to an extremity of a polypeptide or polynucleotide respectively. Such extremity is not limited only to the first or final site of the polypeptide or polynucleotide but may include additional amino acids or nucleotides in the terminal regions. Polypeptide-based molecules may be characterized as having both an N-terminus (terminated by an amino acid with a free amino group (NH₂)) and a C-terminus (terminated by an amino acid with a free carboxyl group (COOH)). Proteins are in some cases made up of multiple polypeptide chains brought together by disulfide bonds or by non-covalent forces (multimers, oligomers). These proteins have multiple N- and C-termini. Alternatively, the termini of the polypeptides may be modified such that they begin or end, as the case may be, with a non-polypeptide based moiety such as an organic conjugate.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein 5, 10, 20, 30, 40, 50, 60, 70, 80, 90, 100 or greater than 100 amino acids in length. In another example, any protein that includes a stretch of 10, 20, 30, 40, 50, or 100 amino acids which are 40%, 50%, 60%, 70%, 80%, 90%, 95%, or 100% identical to any of the sequences described herein can be utilized in accordance with the disclosure. In some embodiments, a polypeptide includes 2, 3, 4, 5, 6, 7, 8, 9, 10, or more mutations as shown in any of the sequences provided or referenced herein. In another example, any protein that includes a stretch of 20, 30, 40, 50, or 100 amino acids that are greater than 80%, 90%, 95%, or 100% identical to any of the sequences described herein, wherein the protein has a stretch of 5, 10, 15, 20, 25, or 30 amino acids that are less than 80%, 75%, 70%, 65% or 60% identical to any of the sequences described herein can be utilized in accordance with the disclosure.

Polypeptide or polynucleotide molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity” as known in the art, refers to a relationship between the sequences of two or more polypeptides or polynucleotides, as determined by comparing the sequences. In the art, identity also means the degree of sequence relatedness between them as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453.). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g. between nucleic acid molecules (e.g. DNA molecules and/or RNA molecules) and/or between polypeptide molecules. Polymeric molecules (e.g. nucleic acid molecules (e.g. DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al, Nucleic Acids Research, 12(1), 387 (1984)), BLASTP, BLASTN, and FASTA Altschul, S. F. et al, J. Molec. Biol., 215, 403 (1990)).

### Chemical Modifications

### Modified Nucleotide Sequences Encoding Epitope Antigen Polypeptides

RNA (e.g., mRNA) vaccines of the present disclosure comprise, in some embodiments, at least one ribonucleic acid (RNA) polynucleotide having an open reading frame encoding at least one respiratory syncytial virus (RSV) antigenic polypeptide, wherein said RNA comprises at least one chemical modification.

The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population. Generally, these terms do not refer to the ribonucleotide modifications in naturally occurring 5′-terminal mRNA cap moieties.

Modifications of polynucleotides include, without limitation, those described herein, and include, but are expressly not limited to, those modifications that comprise chemical modifications. Polynucleotides (e.g., RNA polynucleotides, such as mRNA polynucleotides) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

With respect to a polypeptide, the term “modification” refers to a modification relative to the canonical set 20 amino acids. Polypeptides, as provided herein, are also considered “modified” of they contain amino acid substitutions, insertions or a combination of substitutions and insertions.

Polynucleotides (e.g., RNA polynucleotides, such as mRNA polynucleotides), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

In some embodiments, the polynucleotide (e.g., a RNA, e.g., an mRNA) of the invention comprises a chemically modified nucleobase. The invention includes modified polynucleotides comprising a polynucleotide described herein (e.g., a polynucleotide comprising a nucleotide sequence encoding one or more cancer epitope polypeptides). The modified polynucleotides can be chemically modified and/or structurally modified. When the polynucleotides of the present invention are chemically and/or structurally modified the polynucleotides can be referred to as “modified polynucleotides.”

The present disclosure provides for modified nucleosides and nucleotides of a polynucleotide (e.g., RNA polynucleotides, such as mRNA polynucleotides) encoding one or more cancer epitope polypeptides. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside including a phosphate group. Modified nucleotides can by synthesized by any useful method, such as, for example, chemically, enzymatically, or recombinantly, to include one or more modified or non-natural nucleosides. Polynucleotides can comprise a region or regions of linked nucleosides. Such regions can have variable backbone linkages. The linkages can be standard phosphodiester linkages, in which case the polynucleotides would comprise regions of nucleotides.

The modified polynucleotides disclosed herein can comprise various distinct modifications. In some embodiments, the modified polynucleotides contain one, two, or more (optionally different) nucleoside or nucleotide modifications. In some embodiments, a modified polynucleotide, introduced to a cell can exhibit one or more desirable properties, e.g., improved protein expression, reduced immunogenicity, or reduced degradation in the cell, as compared to an unmodified polynucleotide.

In some embodiments, a polynucleotide of the present invention (e.g., a polynucleotide comprising a nucleotide sequence encoding one or more cancer epitope polypeptides) is structurally modified. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” can be chemically modified to “AT-5meC-G”. The same polynucleotide can be structurally modified from “ATCG” to “ATCCCG”. Here, the dinucleotide “CC” has been inserted, resulting in a structural modification to the polynucleotide.

In some embodiments, the polynucleotides of the present invention are chemically modified. As used herein in reference to a polynucleotide, the terms “chemical modification” or, as appropriate, “chemically modified” refer to modification with respect to adenosine (A), guanosine (G), uridine (U), or cytidine (C) ribo- or deoxyribonucleosides in one or more of their position, pattern, percent or population. Generally, herein, these terms are not intended to refer to the ribonucleotide modifications in naturally occurring 5′-terminal mRNA cap moieties.

In some embodiments, the polynucleotides of the present invention can have a uniform chemical modification of all or any of the same nucleoside type or a population of modifications produced by mere downward titration of the same starting modification in all or any of the same nucleoside type, or a measured percent of a chemical modification of all any of the same nucleoside type but with random incorporation, such as where all uridines are replaced by a uridine analog, e.g., pseudouridine or 5-methoxyuridine. In another embodiment, the polynucleotides can have a uniform chemical modification of two, three, or four of the same nucleoside type throughout the entire polynucleotide (such as all uridines and all cytosines, etc. are modified in the same way).

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those polynucleotides having at least one chemical modification. One example of such non-standard base pairing is the base pairing between the modified nucleotide inosine and adenine, cytosine or uracil. Any combination of base/sugar or linker can be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”s would be substituted for “U”s.

Modifications of polynucleotides (e.g., RNA polynucleotides, such as mRNA polynucleotides), including but not limited to chemical modification, that are useful in the compositions, methods and synthetic processes of the present disclosure include, but are not limited to the following: uniformly nucleotides, nucleosides, and nucleobases: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6, N6 (dimethyl)adenine; N6-cis-hydroxy-isopentenyl-adenosine; α-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-lodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; α-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine; 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethyl cytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynyl cytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; α-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-ethyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethyl aminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methyluridine,), 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uracil; N1-ethyl-pseudo-uracil; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; α-thio-uridine; 1 (aminoalkylamino-carb onylethylenyl)-2(thio)-pseudouracil; 1 (aminoalkylaminocarbonylethylenyl)-2,4-(dithio)pseudouracil; 1 (aminoalkylaminocarbonyl ethylenyl)-4 (thio)pseudouracil; 1 (aminoalkylaminocarbonylethylenyl)-pseudouracil; 1 (aminocarb onylethylenyl)-2(thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2(thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl)pseudo-UTP; 1-Methyl-pseudo-UTP; 1-Ethyl-pseudo-UTP; 2 (thio)pseudouracil; 2′ deoxy uridine; 2′ fluorouridine; 2-(thio)uracil; 2,4-(dithio)psuedouracil; 2′ methyl, 2′amino, 2′azido, 2′fluro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; P seudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (+)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl}pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2(2-ethoxy)-ethoxy}-ethoxy]-ethoxy)-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′amino, 2′azido, 2′fluro-cytidine; 2′ methyl, 2′amino, 2′azido, 2′fluro-adenine; 2′methyl, 2′amino, 2′azido, 2′fluro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; and N6-(19-Amino-pentaoxanonadecyl)adenosine TP.

In some embodiments, the polynucleotide (e.g., RNA polynucleotide, such as mRNA polynucleotide) includes a combination of at least two (e.g., 2, 3, 4 or more) of the aforementioned modified nucleobases.

In some embodiments, the mRNA comprises at least one chemically modified nucleoside. In some embodiments, the at least one chemically modified nucleoside is selected from the group consisting of pseudouridine (ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1ψ), 1-ethyl-pseudouridine (e1ψ), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, 2,8-dimethyladenosine, 2-geranylthiouridine, 2-lysidine, 2-selenouridine, 3-(3-amino-3-carboxypropyl)-5,6-dihydrouridine, 3-(3-amino-3-carboxypropyl)pseudouridine, 3-methylpseudouridine, 5-(carboxyhydroxymethyl)-2′-O-methyluridine methyl ester, 5-aminomethyl-2-geranylthiouridine, 5-aminomethyl-2-selenouridine, 5-aminomethyluridine, 5-carbamoylhydroxymethyluridine, 5-carbamoylmethyl-2-thiouridine, 5-carboxymethyl-2-thiouridine, 5-carboxymethylaminomethyl-2-geranylthiouridine, 5-carboxymethyl aminomethyl-2-selenouridine, 5-cyanomethyluridine, 5-hydroxycytidine, 5-methylaminomethyl-2-geranylthiouridine, 7-aminocarboxypropyl-demethylwyosine, 7-aminocarboxypropylwyosine, 7-aminocarboxypropylwyosine methyl ester, 8-methyladenosine, N4,N4-dimethyl cytidine, N6-formyladenosine, N6-hydroxymethyladenosine, agmatidine, cyclic N6-threonylcarbamoyladenosine, glutamyl-queuosine, methylated undermodified hydroxywybutosine, N4,N4,2′-O-trimethylcytidine, geranylated 5-methylaminomethyl-2-thiouridine, geranylated 5-carboxymethyl aminomethyl-2-thiouridine, Qbase, preQ0base, preQlbase, and two or more combinations thereof. In some embodiments, the at least one chemically modified nucleoside is selected from the group consisting of pseudouridine, 1-methyl-pseudouridine, 1-ethyl-pseudouridine, 5-methylcytosine, 5-methoxyuridine, and a combination thereof. In some embodiments, the polynucleotide (e.g., RNA polynucleotide, such as mRNA polynucleotide) includes a combination of at least two (e.g., 2, 3, 4 or more) of the aforementioned modified nucleobases.

In some embodiments, the mRNA is a uracil-modified sequence comprising an ORF encoding one or more cancer epitope polypeptides, wherein the mRNA comprises a chemically modified nucleobase, e.g., 5-methoxyuracil. In certain aspects of the invention, when the 5-methoxyuracil base is connected to a ribose sugar, as it is in polynucleotides, the resulting modified nucleoside or nucleotide is referred to as 5-methoxyuridine. In some embodiments, uracil in the polynucleotide is at least about 25%, at least about 30%, at least about 40%, at least about 50%, at least about 60%, at least about 70%, at least about 80%, at least 90%, at least 95%, at least 99%, or about 100% 5-methoxyuracil. In one embodiment, uracil in the polynucleotide is at least 95% 5-methoxyuracil. In another embodiment, uracil in the polynucleotide is 100% 5-methoxyuracil.

In embodiments where uracil in the polynucleotide is at least 95% 5-methoxyuracil, overall uracil content can be adjusted such that an mRNA provides suitable protein expression levels while inducing little to no immune response. In some embodiments, the uracil content of the ORF is between about 105% and about 145%, about 105% and about 140%, about 110% and about 140%, about 110% and about 145%, about 115% and about 135%, about 105% and about 135%, about 110% and about 135%, about 115% and about 145%, or about 115% and about 140% of the theoretical minimum uracil content in the corresponding wild-type ORF (% Utm). In other embodiments, the uracil content of the ORF is between about 117% and about 134% or between 118% and 132% of the % UTM. In some embodiments, the uracil content of the ORF encoding one or more cancer epitope polypeptides is about 115%, about 120%, about 125%, about 130%, about 135%, about 140%, about 145%, or about 150% of the % Utm. In this context, the term “uracil” can refer to 5-methoxyuracil and/or naturally occurring uracil.

In some embodiments, the uracil content in the ORF of the mRNA encoding one or more cancer epitope polypeptides of the invention is less than about 50%, about 40%, about 30%, about 20%, about 15%, or about 12% of the total nucleobase content in the ORF. In some embodiments, the uracil content in the ORF is between about 12% and about 25% of the total nucleobase content in the ORF. In other embodiments, the uracil content in the ORF is between about 15% and about 17% of the total nucleobase content in the ORF. In one embodiment, the uracil content in the ORF of the mRNA encoding one or more cancer epitope polypeptides is less than about 20% of the total nucleobase content in the open reading frame. In this context, the term “uracil” can refer to 5-methoxyuracil and/or naturally occurring uracil.

In further embodiments, the ORF of the mRNA encoding one or more cancer epitope polypeptides of the invention comprises 5-methoxyuracil and has an adjusted uracil content containing less uracil pairs (UU) and/or uracil triplets (UUU) and/or uracil quadruplets (UUUU) than the corresponding wild-type nucleotide sequence encoding the one or more cancer epitope polypeptides. In some embodiments, the ORF of the mRNA encoding one or more cancer epitope polypeptides of the invention contains no uracil pairs and/or uracil triplets and/or uracil quadruplets. In some embodiments, uracil pairs and/or uracil triplets and/or uracil quadruplets are reduced below a certain threshold, e.g., no more than 1, 2, 3, 4, 5, 6, 7, 8, 9, 10, 11, 12, 13, 14, 15, 16, 17, 18, 19, or 20 occurrences in the ORF of the mRNA encoding the one or more cancer epitope polypeptides. In a particular embodiment, the ORF of the mRNA encoding the one or more cancer epitope polypeptides of the invention contains less than 20, 19, 18, 17, 16, 15, 14, 13, 12, 11, 10, 9, 8, 7, 6, 5, 4, 3, 2, or 1 non-phenylalanine uracil pairs and/or triplets. In another embodiment, the ORF of the mRNA encoding the one or more cancer epitope polypeptides contains no non-phenylalanine uracil pairs and/or triplets.

In further embodiments, the ORF of the mRNA encoding one or more cancer epitope polypeptides of the invention comprises 5-methoxyuracil and has an adjusted uracil content containing less uracil-rich clusters than the corresponding wild-type nucleotide sequence encoding the one or more cancer epitope polypeptides. In some embodiments, the ORF of the mRNA encoding the one or more cancer epitope polypeptides of the invention contains uracil-rich clusters that are shorter in length than corresponding uracil-rich clusters in the corresponding wild-type nucleotide sequence encoding the one or more cancer epitope polypeptides.

In further embodiments, alternative lower frequency codons are employed. At least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% of the codons in the one or more cancer epitope polypeptides-encoding ORF of the 5-methoxyuracil-comprising mRNA are substituted with alternative codons, each alternative codon having a codon frequency lower than the codon frequency of the substituted codon in the synonymous codon set. The ORF also has adjusted uracil content, as described above. In some embodiments, at least one codon in the ORF of the mRNA encoding the one or more cancer epitope polypeptides is substituted with an alternative codon having a codon frequency lower than the codon frequency of the substituted codon in the synonymous codon set.

In some embodiments, the adjusted uracil content, of the one or more cancer epitope polypeptides-encoding ORF of the 5-methoxyuracil-comprising mRNA exhibits expression levels of the one or more cancer epitope polypeptides when administered to a mammalian cell that are higher than expression levels of the one or more cancer epitope polypeptides from the corresponding wild-type mRNA. In other embodiments, the expression levels of the one or more cancer epitope polypeptides when administered to a mammalian cell are increased relative to a corresponding mRNA containing at least 95% 5-methoxyuracil and having a uracil content of about 160%, about 170%, about 180%, about 190%, or about 200% of the theoretical minimum. In yet other embodiments, the expression levels of the one or more cancer epitope polypeptides when administered to a mammalian cell are increased relative to a corresponding mRNA, wherein at least about 50%, at least about 60%, at least about 70%, at least about 80%, at least about 90%, or about 100% of uracils are 1-methylpseudouracil or pseudouracils. In some embodiments, the mammalian cell is a mouse cell, a rat cell, or a rabbit cell. In other embodiments, the mammalian cell is a monkey cell or a human cell. In some embodiments, the human cell is a HeLa cell, a BJ fibroblast cell, or a peripheral blood mononuclear cell (PBMC). In some embodiments, one or more cancer epitope polypeptides is expressed when the mRNA is administered to a mammalian cell in vivo. In some embodiments, the mRNA is administered to mice, rabbits, rats, monkeys, or humans. In one embodiment, mice are null mice. In some embodiments, the mRNA is administered to mice in an amount of about 0.01 mg/kg, about 0.05 mg/kg, about 0.1 mg/kg, or about 0.15 mg/kg. In some embodiments, the mRNA is administered intravenously or intramuscularly. In other embodiments, the one or more cancer epitope polypeptides is expressed when the mRNA is administered to a mammalian cell in vitro. In some embodiments, the expression is increased by at least about 2-fold, at least about 5-fold, at least about 10-fold, at least about 50-fold, at least about 500-fold, at least about 1500-fold, or at least about 3000-fold. In other embodiments, the expression is increased by at least about 10%, about 20%, about 30%, about 40%, about 50%, 60%, about 70%, about 80%, about 90%, or about 100%.

In some embodiments, adjusted uracil content, one or more cancer epitope polypeptides-encoding ORF of the 5-methoxyuracil-comprising mRNA exhibits increased stability. In some embodiments, the mRNA exhibits increased stability in a cell relative to the stability of a corresponding wild-type mRNA under the same conditions. In some embodiments, the mRNA exhibits increased stability including resistance to nucleases, thermal stability, and/or increased stabilization of secondary structure. In some embodiments, increased stability exhibited by the mRNA is measured by determining the half-life of the mRNA (e.g., in a plasma, cell, or tissue sample) and/or determining the area under the curve (AUC) of the protein expression by the mRNA over time (e.g., in vitro or in vivo). An mRNA is identified as having increased stability if the half-life and/or the AUC is greater than the half-life and/or the AUC of a corresponding wild-type mRNA under the same conditions.

In some embodiments, the mRNA of the present invention induces a detectably lower immune response (e.g., innate or acquired) relative to the immune response induced by a corresponding wild-type mRNA under the same conditions. In other embodiments, the mRNA of the present disclosure induces a detectably lower immune response (e.g., innate or acquired) relative to the immune response induced by an mRNA that encodes for one or more cancer epitope polypeptides but does not comprise 5-methoxyuracil under the same conditions, or relative to the immune response induced by an mRNA that encodes for one or more cancer epitope polypeptides and that comprises 5-methoxyuracil but that does not have adjusted uracil content under the same conditions. The innate immune response can be manifested by increased expression of pro-inflammatory cytokines, activation of intracellular PRRs (RIG-I, MDA5, etc), cell death, and/or termination or reduction in protein translation. In some embodiments, a reduction in the innate immune response can be measured by expression or activity level of Type 1 interferons (e.g., IFN-α, IFN-β, IFN-κ, IFN-δ, IFN-ε, IFN-τ, IFN-ω, and IFN-ζ) or the expression of interferon-regulated genes such as the toll-like receptors (e.g., TLR7 and TLR8), and/or by decreased cell death following one or more administrations of the mRNA of the invention into a cell.

In some embodiments, the expression of Type-1 interferons by a mammalian cell in response to the mRNA of the present disclosure is reduced by at least 10%, 20%, 30%, 40%, 50%, 60%, 70%, 80%, 90%, 95%, 99%, 99.9%, or greater than 99.9% relative to a corresponding wild-type mRNA, to an mRNA that encodes one or more cancer epitope polypeptides but does not comprise 5-methoxyuracil, or to an mRNA that encodes one or more cancer epitope polypeptides and that comprises 5-methoxyuracil but that does not have adjusted uracil content. In some embodiments, the interferon is IFN-β. In some embodiments, cell death frequency cased by administration of mRNA of the present disclosure to a mammalian cell is 10%, 25%, 50%, 75%, 85%, 90%, 95%, or over 95% less than the cell death frequency observed with a corresponding wild-type mRNA, an mRNA that encodes for one or more cancer epitope polypeptides but does not comprise 5-methoxyuracil, or an mRNA that encodes for one or more cancer epitope polypeptides and that comprises 5-methoxyuracil but that does not have adjusted uracil content. In some embodiments, the mammalian cell is a BJ fibroblast cell. In other embodiments, the mammalian cell is a splenocyte. In some embodiments, the mammalian cell is that of a mouse or a rat. In other embodiments, the mammalian cell is that of a human. In one embodiment, the mRNA of the present disclosure does not substantially induce an innate immune response of a mammalian cell into which the mRNA is introduced.

In some embodiments, the polynucleotide is an mRNA that comprises an ORF that encodes one or more cancer epitope polypeptides, wherein uracil in the mRNA is at least about 95% 5-methoxyuracil, wherein the uracil content of the ORF is between about 115% and about 135% of the theoretical minimum uracil content in the corresponding wild-type ORF, and wherein the uracil content in the ORF encoding the one or more cancer epitope polypeptides is less than about 23% of the total nucleobase content in the ORF. In some embodiments, the ORF that encodes the one or more cancer epitope polypeptides is further modified to decrease G/C content of the ORF (absolute or relative) by at least about 40%, as compared to the corresponding wild-type ORF. In yet other embodiments, the ORF encoding the one or more cancer epitope polypeptides contains less than 20 non-phenylalanine uracil pairs and/or triplets. In some embodiments, at least one codon in the ORF of the mRNA encoding the one or more cancer epitope polypeptides is further substituted with an alternative codon having a codon frequency lower than the codon frequency of the substituted codon in the synonymous codon set. In some embodiments, the expression of the one or more cancer epitope polypeptides encoded by an mRNA comprising an ORF wherein uracil in the mRNA is at least about 95% 5-methoxyuracil, and wherein the uracil content of the ORF is between about 115% and about 135% of the theoretical minimum uracil content in the corresponding wild-type ORF, is increased by at least about 10-fold when compared to expression of the one or more cancer epitope polypeptides from the corresponding wild-type mRNA. In some embodiments, the mRNA comprises an open ORF wherein uracil in the mRNA is at least about 95% 5-methoxyuracil, and wherein the uracil content of the ORF is between about 115% and about 135% of the theoretical minimum uracil content in the corresponding wild-type ORF, and wherein the mRNA does not substantially induce an innate immune response of a mammalian cell into which the mRNA is introduced.

In certain embodiments, the chemical modification is at nucleobases in the polynucleotides (e.g., RNA polynucleotide, such as mRNA polynucleotide). In some embodiments, modified nucleobases in the polynucleotide (e.g., RNA polynucleotide, such as mRNA polynucleotide) are selected from the group consisting of 1-methyl-pseudouridine (m1ψ), 1-ethyl-pseudouridine (e1ψ), 5-methoxy-uridine (mo5U), 5-methyl-cytidine (m5C), pseudouridine (ψ), α-thio-guanosine and α-thio-adenosine. In some embodiments, the polynucleotide includes a combination of at least two (e.g., 2, 3, 4 or more) of the aforementioned modified nucleobases.

In some embodiments, the polynucleotide (e.g., RNA polynucleotide, such as mRNA polynucleotide) comprises pseudouridine (ψ) and 5-methyl-cytidine (m5C). In some embodiments, the polynucleotide (e.g., RNA polynucleotide, such as mRNA polynucleotide) comprises 1-methyl-pseudouridine (m1ψ). In some embodiments, the polynucleotide (e.g., RNA polynucleotide, such as mRNA polynucleotide) comprises 1-ethyl-pseudouridine (e1ψ). In some embodiments, the polynucleotide (e.g., RNA polynucleotide, such as mRNA polynucleotide) comprises 1-methyl-pseudouridine (m1ψ) and 5-methyl-cytidine (m5C). In some embodiments, the polynucleotide (e.g., RNA polynucleotide, such as mRNA polynucleotide) comprises 1-ethyl-pseudouridine (e1ψ) and 5-methyl-cytidine (m5C). In some embodiments, the polynucleotide (e.g., RNA polynucleotide, such as mRNA polynucleotide) comprises 2-thiouridine (s2U). In some embodiments, the polynucleotide (e.g., RNA polynucleotide, such as mRNA polynucleotide) comprises 2-thiouridine and 5-methyl-cytidine (m5C). In some embodiments, the polynucleotide (e.g., RNA polynucleotide, such as mRNA polynucleotide) comprises methoxy-uridine (mo5U). In some embodiments, the polynucleotide (e.g., RNA polynucleotide, such as mRNA polynucleotide) comprises 5-methoxy-uridine (mo5U) and 5-methyl-cytidine (m5C). In some embodiments, the polynucleotide (e.g., RNA polynucleotide, such as mRNA polynucleotide) comprises 2′-O-methyl uridine. In some embodiments, the polynucleotide (e.g., RNA polynucleotide, such as mRNA polynucleotide) comprises 2′-O-methyl uridine and 5-methyl-cytidine (m5C). In some embodiments, the polynucleotide (e.g., RNA polynucleotide, such as mRNA polynucleotide) comprises N6-methyl-adenosine (m6A). In some embodiments, the polynucleotide (e.g., RNA polynucleotide, such as mRNA polynucleotide) comprises N6-methyl-adenosine (m6A) and 5-methyl-cytidine (m5C).

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 5-methyl-cytidine (m5C), meaning that all cytosine residues in the mRNA sequence are replaced with 5-methyl-cytidine (m5C). As another example, a polynucleotide can be uniformly modified with 1-methyl-pseudouridine, meaning that all uridine residues in the mRNA sequence are replaced with 1-methyl-pseudouridine. Similarly, a polynucleotide can be uniformly modified for any type of nucleoside residue present in the sequence by replacement with a modified residue such as any of those set forth above.

In some embodiments, the chemically modified nucleosides in the open reading frame are selected from the group consisting of uridine, adenine, cytosine, guanine, and any combination thereof.

In some embodiments, the modified nucleobase is a modified cytosine. Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified uridine. Exemplary nucleobases and nucleosides having a modified uridine include 1-methyl-pseudouridine (m1ψ), 1-ethyl-pseudouridine (e1ψ), 5-methoxy uridine, 2-thio uridine, 5-cyano uridine, 2′-O-methyl uridine, and 4′-thio uridine.

In some embodiments, a modified nucleobase is a modified adenine. Exemplary nucleobases and nucleosides having a modified adenine include 7-deaza-adenine, 1-methyl-adenosine (m1A), 2-methyl-adenine (m2A), N6-methyl-adenosine (m6A), and 2,6-Diaminopurine.

In some embodiments, a modified nucleobase is a modified guanine. Example nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine.

In some embodiments, the nucleobase modified nucleotides in the polynucleotide (e.g., RNA polynucleotide, such as mRNA polynucleotide) are 5-methoxyuridine.

In some embodiments, the polynucleotide (e.g., RNA polynucleotide, such as mRNA polynucleotide) includes a combination of at least two (e.g., 2, 3, 4 or more) of modified nucleobases.

In some embodiments, the polynucleotide (e.g., RNA polynucleotide, such as mRNA polynucleotide) comprises 5-methoxyuridine (5mo5U) and 5-methyl-cytidine (m5C).

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 5-methoxyuridine, meaning that substantially all uridine residues in the mRNA sequence are replaced with 5-methoxyuridine. Similarly, a polynucleotide can be uniformly modified for any type of nucleoside residue present in the sequence by replacement with a modified residue such as any of those set forth above.

In some embodiments, the modified nucleobase is a modified cytosine.

In some embodiments, a modified nucleobase is a modified uracil. Example nucleobases and nucleosides having a modified uracil include 5-methoxyuracil.

In some embodiments, a modified nucleobase is a modified adenine.

In some embodiments, a modified nucleobase is a modified guanine.

In some embodiments, the polynucleotides can include any useful linker between the nucleosides. Such linkers, including backbone modifications, that are useful in the composition of the present disclosure include, but are not limited to the following: 3′-alkylene phosphonates, 3′-amino phosphoramidate, alkene containing backbones, aminoalkylphosphoramidates, aminoalkylphosphotriesters, boranophosphates, —CH₂—O—N(CH₃)—CH₂—, —CH₂—N(CH₃)—N(CH₃)—CH₂—, —CH₂—NH—CH₂—, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH₃)—CH₂—CH₂—, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphorami dates.

The modified nucleosides and nucleotides (e.g., building block molecules), which can be incorporated into a polynucleotide (e.g., RNA or mRNA, as described herein), can be modified on the sugar of the ribonucleic acid. For example, the 2′ hydroxyl group (OH) can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges included methylene, propylene, ether, or amino bridges; aminoalkyl, as defined herein; aminoalkoxy, as defined herein; amino as defined herein; and amino acid, as defined herein

Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar. Such sugar modifications are taught International Patent Publication Nos. WO2013052523 and WO2014093924, the contents of each of which are incorporated herein by reference in their entireties.

The polynucleotides of the invention (e.g., a polynucleotide comprising a nucleotide sequence encoding one or more cancer epitope polypeptides or a functional fragment or variant thereof) can include a combination of modifications to the sugar, the nucleobase, and/or the internucleoside linkage. These combinations can include any one or more modifications described herein.

The polynucleotides of the present disclosure may be partially or fully modified along the entire length of the molecule. For example, one or more or all or a given type of nucleotide (e.g., purine or pyrimidine, or any one or more or all of A, G, U, C) may be uniformly modified in a polynucleotide of the invention, or in a given predetermined sequence region thereof (e.g., in the mRNA including or excluding the polyA tail). In some embodiments, all nucleotides X in a polynucleotide of the present disclosure (or in a given sequence region thereof) are modified nucleotides, wherein X may any one of nucleotides A, G, U, C, or any one of the combinations A+G, A+U, A+C, G+U, G+C, U+C, A+G+U, A+G+C, G+U+C or A+G+C.

The polynucleotide may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The polynucleotides may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the polynucleotides may contain a modified pyrimidine such as a modified uracil or cytosine. In some embodiments, at least 5%, at least 10%, at least 25%, at least 50%, at least 80%, at least 90% or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

Thus, in some embodiments, the RNA vaccines comprise a 5′UTR element, an optionally codon optimized open reading frame, and a 3′UTR element, a poly(A) sequence and/or a polyadenylation signal wherein the RNA is not chemically modified.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridineor 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵ s2U), 5-aminomethyl-2-thio-uridine (nm⁵s²U), 5-methylaminomethyl-uridine (mnm⁵U), 5-methyl aminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethyl aminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(m⁵ s2U), 1-taurinomethyl-4-thio-pseudouridine, 5-methyl-uridine (m⁵U, i.e., having the nucleobase deoxythymine), 1-methyl-pseudouridine (m¹ψ), 1-ethyl-pseudouridine (e1ψ), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenyl amino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms²m⁶A), N6-isopentenyl-adenosine (i⁶A), 2-methylthio-N6-isopentenyl-adenosine (ms²i⁶A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶₂Am), 1,2′-O-dimethyl-adenosine (m Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²₂G), N2,7-dimethyl-guanosine (m^{2,7}G), N2, N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²₂Gm), 1-methyl-2′-O-methyl-guanosine (m Gm), N2,7-dimethyl-2′-O-methyl-guanosine (m^{2,7}Gm), 2′-O-methyl-inosine (Im), 1,2′-O-dimethyl-inosine (m¹Im), 2′-O-ribosylguanosine (phosphate) (Gr(p)), 1-thio-guanosine, 06-methyl-guanosine, 2′-F-ara-guanosine, and 2′-F-guanosine.

In Vitro Transcription of RNA (e.g., mRNA)

Cancer vaccines of the present disclosure comprise at least one RNA polynucleotide, such as a mRNA (e.g., modified mRNA). mRNA, for example, is transcribed in vitro from template DNA, referred to as an “in vitro transcription template.” In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

In some embodiments, a polynucleotide includes 200 to 3,000 nucleotides. For example, a polynucleotide may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

In other aspects, the invention relates to a method for preparing an mRNA cancer vaccine by IVT methods. In vitro transcription (IVT) methods permit template-directed synthesis of RNA molecules of almost any sequence. The size of the RNA molecules that can be synthesized using IVT methods range from short oligonucleotides to long nucleic acid polymers of several thousand bases. IVT methods permit synthesis of large quantities of RNA transcript (e.g., from microgram to milligram quantities) (Beckert et al., Synthesis of RNA by in vitro transcription, Methods Mol Biol. 703:29-41(2011); Rio et al. RNA: A Laboratory Manual. Cold Spring Harbor: Cold Spring Harbor Laboratory Press, 2011, 205-220.; Cooper, Geoffery M. The Cell: A Molecular Approach. 4th ed. Washington D.C.: ASM Press, 2007. 262-299). Generally, IVT utilizes a DNA template featuring a promoter sequence upstream of a sequence of interest. The promoter sequence is most commonly of bacteriophage origin (ex. the T7, T3 or SP6 promoter sequence) but many other promotor sequences can be tolerated including those designed de novo. Transcription of the DNA template is typically best achieved by using the RNA polymerase corresponding to the specific bacteriophage promoter sequence. Exemplary RNA polymerases include, but are not limited to T7 RNA polymerase, T3 RNA polymerase, or SP6 RNA polymerase, among others. IVT is generally initiated at a dsDNA but can proceed on a single strand.

It will be appreciated that mRNA vaccines of the present disclosure, e.g., mRNAs encoding the cancer antigen or e.g., activating oncogene mutation peptide, may be made using any appropriate synthesis method. For example, in some embodiments, mRNA vaccines of the present disclosure are made using IVT from a single bottom strand DNA as a template and complementary oligonucleotide that serves as promotor. The single bottom strand DNA may act as a DNA template for in vitro transcription of RNA, and may be obtained from, for example, a plasmid, a PCR product, or chemical synthesis. In some embodiments, the single bottom strand DNA is linearized from a circular template. The single bottom strand DNA template generally includes a promoter sequence, e.g., a bacteriophage promoter sequence, to facilitate IVT. Methods of making RNA using a single bottom strand DNA and a top strand promoter complementary oligonucleotide are known in the art. An exemplary method includes, but is not limited to, annealing the DNA bottom strand template with the top strand promoter complementary oligonucleotide (e.g., T7 promoter complementary oligonucleotide, T3 promoter complementary oligonucleotide, or SP6 promoter complementary oligonucleotide), followed by IVT using an RNA polymerase corresponding to the promoter sequence, e.g., aT7 RNA polymerase, a T3 RNA polymerase, or an SP6 RNA polymerase.

IVT methods can also be performed using a double-stranded DNA template. For example, in some embodiments, the double-stranded DNA template is made by extending a complementary oligonucleotide to generate a complementary DNA strand using strand extension techniques available in the art. In some embodiments, a single bottom strand DNA template containing a promoter sequence and sequence encoding one or more epitopes of interest is annealed to a top strand promoter complementary oligonucleotide and subjected to a PCR-like process to extend the top strand to generate a double-stranded DNA template. Alternatively or additionally, a top strand DNA containing a sequence complementary to the bottom strand promoter sequence and complementary to the sequence encoding one or more epitopes of interest is annealed to a bottom strand promoter oligonucleotide and subjected to a PCR-like process to extend the bottom strand to generate a double-stranded DNA template. In some embodiments, the number of PCR-like cycles ranges from 1 to 20 cycles, e.g., 3 to 10 cycles. In some embodiments, a double-stranded DNA template is synthesized wholly or in part by chemical synthesis methods. The double-stranded DNA template can be subjected to in vitro transcription as described herein.

In another aspect, mRNA vaccines of the present disclosure, e.g., mRNAs encoding the cancer antigen or eptiope, may be made using two DNA strands that are complementary across an overlapping portion of their sequence, leaving single-stranded overhangs (i.e., sticky ends) when the complementary portions are annealed. These single-stranded overhangs can be made double-stranded by extending using the other strand as a template, thereby generating double-stranded DNA. In some cases, this primer extension method can permit larger ORFs to be incorporated into the template DNA sequence, e.g., as compared to sizes incorporated into the template DNA sequences obtained by top strand DNA synthesis methods. In the primer extension method, a portion of the 3′-end of a first strand (in the 5″-3′ direction) is complementary to a portion the 3′-end of a second strand (in the 3′-5′ direction). In some such embodiments, the single first strand DNA may include a sequence of a promoter (e.g., T7, T3, or SP6), optionally a 5′-UTR, and some or all of an ORF (e.g., a portion of the 5′-end of the ORF). In some embodiments, the single second strand DNA may include complementary sequences for some or all of an ORF (e.g., a portion complementary to the 3′-end of the ORF), and optionally a 3′-UTR, a stop sequence, and/or a poly(A) tail. Methods of making RNA using two synthetic DNA strands may include annealing the two strands with overlapping complementary portions, followed by primer extension using one or more PCR-like cycles to extend the strands to generate a double-stranded DNA template. In some embodiments, the number of PCR-like cycles ranges from 1 to 20 cycles, e.g., 3 to 10 cycles. Such double-stranded DNA can be subjected to in vitro transcription as described herein.

In another aspect, mRNA vaccines of the present disclosure, e.g., mRNAs encoding the cancer antigen or eptiope, may be made using synthetic double-stranded linear DNA molecules, such as gBlocks® (Integrated DNA Technologies, Coralville, Iowa), as the double-stranded DNA template. An advantage to such synthetic double-stranded linear DNA molecules is that they provide a longer template from which to generate mRNAs. For example, gBlocks® can range in size from 45-1000 (e.g., 125-750 nucleotides). In some embodiments, a synthetic double-stranded linear DNA template includes a full length 5′-UTR, a full length 3′-UTR, or both. A full length 5′-UTR may be up to 100 nucleotides in length, e.g., about 40-60 nucleotides. A full length 3′-UTR may be up to 300 nucleotides in length, e.g., about 100-150 nucleotides.

To facilitate generation of longer constructs, two or more double-stranded linear DNA molecules and/or gene fragments that are designed with overlapping sequences on the 3′ strands may be assembled together using methods known in art. For example, the Gibson Assembly™ Method (Synthetic Genomics, Inc., La Jolla, Calif.) may be performed with the use of a mesophilic exonuclease that cleaves bases from the 5′-end of the double-stranded DNA fragments, followed by annealing of the newly formed complementary single-stranded 3′-ends, polymerase-dependent extension to fill in any single-stranded gaps, and finally, covalent joining of the DNA segments by a DNA ligase.

In another aspect, mRNA vaccines of the present disclosure, e.g., mRNAs encoding the cancer antigen or epitope, may be made using chemical synthesis of the RNA. Methods, for instance, involve annealing a first polynucleotide comprising an open reading frame encoding the polypeptide and a second polynucleotide comprising a 5′-UTR to a complementary polynucleotide conjugated to a solid support. The 3′-terminus of the second polynucleotide is then ligated to the 5′-terminus of the first polynucleotide under suitable conditions. Suitable conditions include the use of a DNA Ligase. The ligation reaction produces a first ligation product. The 5′ terminus of a third polynucleotide comprising a 3′-UTR is then ligated to the 3′-terminus of the first ligation product under suitable conditions. Suitable conditions for the second ligation reaction include an RNA Ligase. A second ligation product is produced in the second ligation reaction. The second ligation product is released from the solid support to produce an mRNA encoding a polypeptide of interest. In some embodiments the mRNA is between 30 and 1000 nucleotides.

An mRNA encoding a polypeptide of interest may also be prepared by binding a first polynucleotide comprising an open reading frame encoding the polypeptide to a second polynucleotide comprising 3′-UTR to a complementary polynucleotide conjugated to a solid support. The 5′-terminus of the second polynucleotide is ligated to the 3′-terminus of the first polynucleotide under suitable conditions. The suitable conditions include a DNA Ligase. The method produces a first ligation product. A third polynucleotide comprising a 5′-UTR is ligated to the first ligation product under suitable conditions to produce a second ligation product. The suitable conditions include an RNA Ligase, such as T4 RNA. The second ligation product is released from the solid support to produce an mRNA encoding a polypeptide of interest.

In some embodiments the first polynucleotide features a 5′-triphosphate and a 3′-OH. In other embodiments the second polynucleotide comprises a 3′-OH. In yet other embodiments, the third polynucleotide comprises a 5′-triphosphate and a 3′-OH. The second polynucleotide may also include a 5′-cap structure. The method may also involve the further step of ligating a fourth polynucleotide comprising a poly-A region at the 3′-terminus of the third polynucleotide. The fourth polynucleotide may comprise a 5′-triphosphate.

The method may or may not comprise reverse phase purification. The method may also include a washing step wherein the solid support is washed to remove unreacted polynucleotides. The solid support may be, for instance, a capture resin. In some embodiments the method involves dT purification.

In accordance with the present disclosure, template DNA encoding the mRNA vaccines of the present disclosure includes an open reading frame (ORF) encoding one or more cancer epitopes. In some embodiments, the template DNA includes an ORF of up to 1000 nucleotides, e.g., about 10-350, 30-300 nucleotides or about 50-250 nucleotides. In some embodiments, the template DNA includes an ORF of about 150 nucleotides. In some embodiments, the template DNA includes an ORF of about 200 nucleotides.

In some embodiments, IVT transcripts are purified from the components of the IVT reaction mixture after the reaction takes place. For example, the crude IVT mix may be treated with RNase-free DNase to digest the original template. The mRNA can be purified using methods known in the art, including but not limited to, precipitation using an organic solvent or column based purification method. Commercial kits are available to purify RNA, e.g., MEGACLEAR™ Kit (Ambion, Austin, Tex.). The mRNA can be quantified using methods known in the art, including but not limited to, commercially available instruments, e.g., NanoDrop. Purified mRNA can be analyzed, for example, by agarose gel electrophoresis to confirm the RNA is the proper size and/or to confirm that no degradation of the RNA has occurred.

Untranslated Regions (UTRs)

Untranslated regions (UTRs) are nucleic acid sections of a polynucleotide before a start codon (5′UTR) and after a stop codon (3′UTR) that are not translated. In some embodiments, a polynucleotide (e.g., a ribonucleic acid (RNA), e.g., a messenger RNA (mRNA)) of the invention comprising an open reading frame (ORF) encoding one or more cancer antigen or epitope further comprises UTR (e.g., a 5′UTR or functional fragment thereof, a 3′UTR or functional fragment thereof, or a combination thereof).

A UTR can be homologous or heterologous to the coding region in a polynucleotide. In some embodiments, the UTR is homologous to the ORF encoding the one or more cancer epitope polypeptides. In some embodiments, the UTR is heterologous to the ORF encoding the one or more cancer epitope polypeptides. In some embodiments, the polynucleotide comprises two or more 5′UTRs or functional fragments thereof, each of which have the same or different nucleotide sequences. In some embodiments, the polynucleotide comprises two or more 3′UTRs or functional fragments thereof, each of which have the same or different nucleotide sequences.

In some embodiments, the 5′UTR or functional fragment thereof, 3′ UTR or functional fragment thereof, or any combination thereof is sequence optimized.

In some embodiments, the 5′UTR or functional fragment thereof, 3′ UTR or functional fragment thereof, or any combination thereof comprises at least one chemically modified nucleobase, e.g., 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′UTR or 3′UTR comprises one or more regulatory features of a full length 5′ or 3′ UTR, respectively.

Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG (SEQ ID NO: 246), where R is a purine (adenine or guanine) three bases upstream of the start codon (AUG), which is followed by another ‘G’. 5′UTRs also have been known to form secondary structures that are involved in elongation factor binding.

By engineering the features typically found in abundantly expressed genes of specific target organs, one can enhance the stability and protein production of a polynucleotide. For example, introduction of 5′UTR of liver-expressed mRNA, such as albumin, serum amyloid A, Apolipoprotein A/B/E, transferrin, alpha fetoprotein, erythropoietin, or Factor VIII, can enhance expression of polynucleotides in hepatic cell lines or liver. Likewise, use of 5′UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

In some embodiments, the 5′UTR and the 3′UTR can be heterologous. In some embodiments, the 5′UTR can be derived from a different species than the 3′UTR. In some embodiments, the 3′UTR can be derived from a different species than the 5′UTR.

Co-owned International Patent Application No. PCT/US2014/021522 (Publ. No. WO/2014/164253, incorporated herein by reference in its entirety) provides a listing of exemplary UTRs that can be utilized in the polynucleotide of the present invention as flanking regions to an ORF.

Exemplary UTRs of the application include, but are not limited to, one or more 5′UTR and/or 3′UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone e (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

Other exemplary 5′ and 3′ UTRs include, but are not limited to, those described in Karikó et al, Mol. Ther. 2008 16(11):1833-1840; Karikó et al., Mol. Ther. 2012 20(5):948-953; Karikó et al, Nucleic Acids Res. 2011 39(21):e142; Strong et al., Gene Therapy 1997 4:624-627; Hansson et al., J. Biol. Chem. 2015 290(9):5661-5672; Yu et al, Vaccine 2007 25(10):1701-1711; Cafri et al., Mol. Ther. 2015 23(8):1391-1400; Andries et al., Mol. Pharm. 2012 9(8):2136-2145; Crowley et al., Gene Ther. 2015 Jun. 30, doi:10.1038/gt.2015.68; Ramunas et al., FASEB J. 2015 29(5):1930-1939; Wang et al., Curr. Gene Ther. 2015 15(4):428-435; Holtkamp et al., Blood 2006 108(13):4009-4017; Kormann et al, Nat. Biotechnol. 2011 29(2):154-157; Poleganov et al., Hum. Gen. Ther. 2015 26(11):751-766; Warren et al., Cell Stem Cell 2010 7(5):618-630; Mandal and Rossi, Nat. Protoc. 2013 8(3):568-582; Holcik and Liebhaber, PNAS 1997 94(6):2410-2414; Ferizi et al., Lab Chip. 2015 15(17):3561-3571; Thess et al., Mol. Ther. 2015 23(9): 1456-1464; Boros et al., PLoS One 2015 10(6):e0131141; Boros et al., J. Photochem. Photobiol. B. 2013 129:93-99; Andries et al., J. Control. Release 2015 217:337-344; Zinckgraf et al, Vaccine 2003 21(15): 1640-9; Garneau et al., J. Virol. 2008 82(2):880-892; Holden and Harris, Virology 2004 329(1):119-133; Chiu et al, J. Virol. 2005 79(13):8303-8315; Wang et al, EMBO J. 1997 16(13):4107-4116; Al-Zoghaibi et al, Gene 2007 391(1-2):130-9; Vivinus et al, Eur. J. Biochem. 2001 268(7):1908-1917; Gan and Rhoads, J. 5 Biol. Chem. 1996 271(2):623-626; Boado et al, J. Neurochem. 1996 67(4): 1335-1343; Knirsch and Clerch, Biochem. Biophys. Res. Commun. 2000 272(1):164-168; Chung et al, Biochemistry 1998 37(46):16298-16306; Izquierdo and Cuevza, Biochem. J. 2000 346 Pt 3:849-855; Dwyer et at, J. Neurochem. 1996 66(2):449-458; Black et al, Mol. Cell. Biol. 1997 17(5):2756-2763; Izquierdo and Cuevza, Mol. Cell. Biol. 1997 17(9):5255-5268; U.S. Pat. Nos. 8,278,036; 8,748,089; 8,835,108; 9,012,219; US2010/0129877; US2011/0065103; US2011/0086904; US2012/0195936; US2014/020675; US2013/0195967; US2014/029490; US2014/0206753; WO2007/036366; WO2011/015347; WO2012/072096; WO2013/143555; WO2014/071963; WO2013/185067; WO2013/182623; WO2014/089486; WO2013/185069; WO2014/144196; WO2014/152659; 2014/152673; WO2014/152940; WO2014/152774; WO2014/153052; WO2014/152966, WO2014/152513; WO2015/101414; WO2015/101415; WO2015/062738; and WO2015/024667; the contents of each of which are incorporated herein by reference in their entirety.

In some embodiments, the 5′UTR is selected from the group consisting of a (3-globin 5′UTR; a 5′UTR containing a strong Kozak translational initiation signal; a cytochrome b-245α polypeptide (CYBA) 5′UTR; a hydroxysteroid (17-13) dehydrogenase (HSD17B4) 5′UTR; a Tobacco etch virus (TEV) 5′UTR; a Venezuelen equine encephalitis virus (TEEV) 5′UTR; a 5′ proximal open reading frame of rubella virus (RV) RNA encoding nonstructural proteins; a Dengue virus (DEN) 5′UTR; a heat shock protein 70 (Hsp70) 5′UTR; a eIF4G 5′UTR; a GLUT1 5′UTR; functional fragments thereof and any combination thereof.

In some embodiments, the 3′UTR is selected from the group consisting of a β-globin 3′UTR; a CYBA 3′UTR; an albumin 3′UTR; a growth hormone (GH) 3′UTR; a VEEV 3′UTR; a hepatitis B virus (HBV) 3′UTR; α-globin 3′UTR; a DEN 3′UTR; a PAV barley yellow dwarf virus (BYDV-PAV) 3′UTR; an elongation factor 1 al (EEF1A1) 3′UTR; a manganese superoxide dismutase (MnSOD) 3′UTR; a β subunit of mitochondrial H(+)-ATP synthase (β-mRNA) 3′UTR; a GLUT1 3′UTR; a MEF2A 3′UTR; a β-F1-ATPase 3′UTR; functional fragments thereof and combinations thereof.

Other exemplary UTRs include, but are not limited to, one or more of the UTRs, including any combination of UTRs, disclosed in WO2014/164253, the contents of which are incorporated herein by reference in their entirety. Shown in Table 21 of U.S. Provisional Application No. 61/775,509 and in Table 22 of U.S. Provisional Application No. 61/829,372, the contents of each are incorporated herein by reference in their entirety, is a listing start and stop sites for 5′UTRs and 3′UTRs. In Table 21, each 5′UTR (5′-UTR-005 to 5′-UTR 68511) is identified by its start and stop site relative to its native or wild-type (homologous) transcript (ENST; the identifier used in the ENSEMBL database).

Wild-type UTRs derived from any gene or mRNA can be incorporated into the polynucleotides of the invention. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, and sequences available at www.addgene.org/Derrick_Rossi/, the contents of each are incorporated herein by reference in their entirety. UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the polynucleotide comprises multiple UTRs, e.g., a double, a triple or a quadruple 5′UTR or 3′UTR. For example, a double UTR comprises two copies of the same UTR either in series or substantially in series. For example, a double beta-globin 3′UTR can be used (see US2010/0129877, the contents of which are incorporated herein by reference in its entirety).

In certain embodiments, the polynucleotides of the invention comprise a 5′UTR and/or a 3′UTR selected from any of the UTRs disclosed herein. In some embodiments, the 5′UTR and/or the 3′ UTR comprise:

| Name | SEQ ID NO: |
|---|---|
| 5′UTR-001 (Upstream UTR) | 247 |
| 5′UTR-002 (Upstream UTR) | 248 |
| 5′UTR-003 (Upstream UTR) | 249 |
| 5′UTR-004 (Upstream UTR) | 250 |
| 5′UTR-005 (Upstream UTR) | 251 |
| 5′UTR-006 (Upstream UTR) | 252 |
| 5′UTR-007 (Upstream UTR) | 253 |
| 5′UTR-008 (Upstream UTR) | 254 |
| 5′UTR-009 (Upstream UTR) | 255 |
| 5′UTR-010 (Upstream UTR) | 256 |
| 5′UTR-011 (Upstream UTR) | 257 |
| 5′UTR-012 (Upstream UTR) | 258 |
| 5′UTR-013 (Upstream UTR) | 259 |
| 5′UTR-014 (Upstream UTR) | 260 |
| 5′UTR-015 (Upstream UTR) | 261 |
| 5′UTR-016 (Upstream UTR) | 262 |
| 5′UTR-017 (Upstream UTR) | 263 |
| 5′UTR-018 (Upstream UTR) | 264 |
| 142-3p 5′UTR-001 (Upstream UTR including miR142-3p | 265 |
| binding site) | |
| 142-3p 5′UTR-002 (Upstream UTR including miR142-3p | 266 |
| binding site) | |
| 142-3p 5′UTR-003 (Upstream UTR including miR142-3p | 267 |
| binding site) | |
| 142-3p 5′UTR-004 (Upstream UTR including miR142-3p | 268 |
| binding site) | |
| 142-3p 5′UTR-005 (Upstream UTR including miR142-3p | 269 |
| binding site) | |
| 142-3p 5′UTR-006 (Upstream UTR including miR142-3p | 270 |
| binding site) | |
| 142-3p 5′UTR-007 (Upstream UTR including miR142-3p | 271 |
| binding site) | |
| 3′UTR comprises: 3′UTR-001 (Creatine Kinase UTR) | 272 |
| 3′UTR-002 (Myoglobin UTR) | 273 |
| 3′UTR-003 (α-actin UTR) | 274 |
| 3′UTR-004 (Albumin UTR) | 275 |
| 3′UTR-005 (α-globin UTR) | 276 |
| 3′UTR-006 (G-CSF UTR) | 277 |
| 3′UTR-007 (Col1a2; collagen, type I, alpha 2 UTR) | 278 |
| 3′UTR-008 (Col6a2; collagen, type VI, alpha 2 UTR) | 279 |
| 3′UTR-009 (RPN1; ribophorin I UTR) | 280 |
| 3′UTR-010 (LRP1; low density lipoprotein receptor- | 281 |
| related protein 1 UTR) | |
| 3′UTR-011 (Nnt1; cardiotrophin-like cytokine factor | 282 |
| 1 UTR) | |
| 3′UTR-012 (Col6a1; collagen, type VI, alpha 1 UTR) | 283 |
| 3′UTR-013 (Calr; calreticulin UTR) | 284 |
| 3′UTR-014 (Col1a1; collagen, type I, alpha 1 UTR) | 285 |
| 3′UTR-015 (Plod1; procollagen-lysine, 2-oxoglutarate | 286 |
| 5-dioxygenase 1 UTR) | |
| 3′UTR-016 (Nucb1; nucleobindin 1 UTR) | 287 |
| 3′UTR-017 (α-globin) | 288 |
| 3′UTR-018 | 289 |
| 3′ UTR with miR 142-3p binding site | 290 |
| 3′ UTR with miR 126-3p binding site | 291 |
| 3′ UTR with miR 142-3p and miR 126-3p binding sites | 292 |
| 3′ UTR with 3 miR 142-3p binding sites | 293 |
| 3′UTR with miR 142-5p binding site | 294 |
| 3′UTR with 3 miR 142-5p binding sites | 295 |
| 3′UTR with 2 miR 142-5p binding sites and 1 miR | 296 |
| 142-3p binding site | |
| 3′UTR with miR 142-3p binding site, P1 insertion | 297 |
| 3′UTR with miR 142-3p binding site, P2 insertion | 298 |
| 3′UTR with miR 142-3p binding site, P3 insertion | 299 |
| 3′UTR with miR 155-5p binding site | 300 |
| 3′ UTR with 3 miR 155-5p binding sites | 301 |
| 3′UTR with 2 miR 155-5p binding sites and 1 miR | 302 |
| 142-3p binding site | |

In certain embodiments, the 5′UTR and/or 3′UTR sequence of the invention comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of 5′UTR sequences comprising any of SEQ ID NOs: 247-271 and/or 3′UTR sequences comprises any of SEQ ID NOs: 272-302, and any combination thereof.

The polynucleotides of the invention can comprise combinations of features. For example, the ORF can be flanked by a 5′UTR that comprises a strong Kozak translational initiation signal and/or a 3′UTR comprising an oligo(dT) sequence for templated addition of a poly-A tail. A 5′UTR can comprise a first polynucleotide fragment and a second polynucleotide fragment from the same and/or different UTRs (see, e.g., US2010/0293625, herein incorporated by reference in its entirety).

It is also within the scope of the present invention to have patterned UTRs. As used herein “patterned UTRs” include a repeating or alternating pattern, such as ABABAB or AABBAABBAABB or ABCABCABC or variants thereof repeated once, twice, or more than 3 times. In these patterns, each letter, A, B, or C represent a different UTR nucleic acid sequence.

Other non-UTR sequences can be used as regions or subregions within the polynucleotides of the invention. For example, introns or portions of intron sequences can be incorporated into the polynucleotides of the invention. Incorporation of intronic sequences can increase protein production as well as polynucleotide expression levels. In some embodiments, the polynucleotide of the invention comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide of the invention comprises 5′ and/or 3′ sequence associated with the 5′ and/or 3′ ends of rubella virus (RV) genomic RNA, respectively, or deletion derivatives thereof, including the 5′ proximal open reading frame of RV RNA encoding nonstructural proteins (e.g., see Pogue et al., J. Virol. 67(12):7106-7117, the contents of which are incorporated herein by reference in their entirety). Viral capsid sequences can also be used as a translational enhancer, e.g., the 5′ portion of a capsid sequence, (e.g., semliki forest virus and sindbis virus capsid RNAs as described in Sjoberg et al, Biotechnology (NY) 1994 12(11): 1127-1131, and Frolov and Schlesinger J. Virol. 1996 70(2): 1182-1190, the contents of each of which are incorporated herein by reference in their entirety). In some embodiments, the polynucleotide comprises an IRES instead of a 5′UTR sequence. In some embodiments, the polynucleotide comprises an ORF and a viral capsid sequence. In some embodiments, the polynucleotide comprises a synthetic 5′UTR in combination with a non-synthetic 3′UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can include those described in US2009/0226470, incorporated herein by reference in its entirety, and others known in the art. As a non-limiting example, the TEE can be located between the transcription promoter and the start codon. In some embodiments, the 5′UTR comprises a TEE.

In one aspect, a TEE is a conserved element in a UTR that can promote translational activity of a nucleic acid such as, but not limited to, cap-dependent or cap-independent translation. The conservation of these sequences has been shown across 14 species including humans. See, e.g., Panek et al, “An evolutionary conserved pattern of 18S rRNA sequence complementarity to mRNA 5′UTRs and its implications for eukaryotic gene translation regulation,” Nucleic Acids Research 2013, doi:10.1093/nar/gkt548, incorporated herein by reference in its entirety.

In one non-limiting example, the TEE comprises the TEE sequence in the 5′-leader of the Gtx homeodomain protein. See Chappell et al, PNAS 2004 101:9590-9594, incorporated herein by reference in its entirety.

In another non-limiting example, the TEE comprises a TEE having one or more of the sequences of SEQ ID NOs: 1-35 in US2009/0226470, US2013/0177581, and WO2009/075886; SEQ ID NOs: 1-5 and 7-645 in WO2012/009644; and SEQ ID NO: 1 WO1999/024595, U.S. Pat. Nos. 6,310,197, and 6,849,405; the contents of each of which are incorporated herein by reference in their entirety.

In some embodiments, the TEE is an internal ribosome entry site (IRES), HCV-IRES, or an IRES element such as, but not limited to, those described in: U.S. Pat. No. 7,468,275, US2007/0048776, US2011/0124100, WO2007/025008, and WO2001/055369; the contents of each of which re incorporated herein by reference in their entirety. The IRES elements can include, but are not limited to, the Gtx sequences (e.g., Gtx9-nt, Gtx8-nt, Gtx7-nt) as described by Chappell et at, PNAS 2004 101:9590-9594, Zhou et al, PNAS 2005 102:6273-6278, US2007/0048776, US2011/0124100, and WO2007/025008; the contents of each of which are incorporated herein by reference in their entirety.

“Translational enhancer polynucleotide” or “translation enhancer polynucleotide sequence” refer to a polynucleotide that includes one or more of the TEE provided herein and/or known in the art (see. e.g., U.S. Pat. Nos. 6,310,197, 6,849,405, 7,456,273, 7,183,395, US2009/0226470, US2007/0048776, US2011/0124100, US2009/0093049, US2013/0177581, WO2009/075886, WO2007/025008, WO2012/009644, WO2001/055371, WO1999/024595, EP2610341A1, and EP2610340A1; the contents of each of which are incorporated herein by reference in their entirety), or their variants, homologs, or functional derivatives. In some embodiments, the polynucleotide of the invention comprises one or multiple copies of a TEE. The TEE in a translational enhancer polynucleotide can be organized in one or more sequence segments. A sequence segment can harbor one or more of the TEEs provided herein, with each TEE being present in one or more copies. When multiple sequence segments are present in a translational enhancer polynucleotide, they can be homogenous or heterogeneous. Thus, the multiple sequence segments in a translational enhancer polynucleotide can harbor identical or different types of the TEE provided herein, identical or different number of copies of each of the TEE, and/or identical or different organization of the TEE within each sequence segment. In one embodiment, the polynucleotide of the invention comprises a translational enhancer polynucleotide sequence.

In some embodiments, a 5′UTR and/or 3′UTR of a polynucleotide of the invention comprises at least one TEE or portion thereof that is disclosed in: WO1999/024595, WO2012/009644, WO2009/075886, WO2007/025008, WO1999/024595, WO2001/055371, EP2610341A1, EP2610340A1, U.S. Pat. Nos. 6,310,197, 6,849,405, 7,456,273, 7,183,395, US2009/0226470, US2011/0124100, US2007/0048776, US2009/0093 049, or US2013/0177581, the contents of each are incorporated herein by reference in their entirety.

In some embodiments, a 5′UTR and/or 3′UTR of a polynucleotide of the invention comprises a TEE that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to a TEE disclosed in: US2009/0226470, US2007/0048776, US2013/0177581, US2011/0124100, WO1999/024595, WO2012/009644, WO2009/075886, WO2007/025008, EP2610341A1, EP2610340A1, U.S. Pat. Nos. 6,310,197, 6,849,405, 7,456,273, 7,183,395, Chappell et al, PNAS 2004 101:9590-9594, Zhou et al, PNAS 2005 102:6273-6278, and Supplemental Table 1 and in Supplemental Table 2 of Wellensiek et al., “Genome-wide profiling of human cap-independent translation-enhancing elements,” Nature Methods 2013, DOI:10.1038/NMETH.2522; the contents of each of which are incorporated herein by reference in their entirety.

In some embodiments, a 5′UTR and/or 3′UTR of a polynucleotide of the invention comprises a TEE which is selected from a 5-30 nucleotide fragment, a 5-25 nucleotide fragment, a 5-20 nucleotide fragment, a 5-15 nucleotide fragment, or a 5-10 nucleotide fragment (including a fragment of 5, 6, 7, 8, 9, 10, 11, 12, 13, 14, 15, 16, 17, 18, 19, 20, 21, 22, 23, 24, 25, 26, 27, 28, 29, or 30 nucleotides) of a TEE sequence disclosed in: US2009/0226470, US2007/0048776, US2013/0177581, US2011/0124100, WO1999/024595, WO2012/009644, WO2009/075886, WO2007/025008, EP2610341A1, EP2610340A1, U.S. Pat. Nos. 6,310,197, 6,849,405, 7,456,273, 7,183,395, Chappell et al, PNAS 2004 101:9590-9594, Zhou et al, PNAS 2005 102:6273-6278, and Supplemental Table 1 and in Supplemental Table 2 of Wellensiek et al, “Genome-wide profiling of human cap-independent translation-enhancing elements,” Nature Methods 2013, DOI: 10.1038/NMETH.2522.

In some embodiments, a 5′UTR and/or 3′UTR of a polynucleotide of the invention comprises a TEE which is a transcription regulatory element described in any of U.S. Pat. Nos. 7,456,273, 7,183,395, US2009/0093049, and WO2001/055371, the contents of each of which are incorporated herein by reference in their entirety. The transcription regulatory elements can be identified by methods known in the art, such as, but not limited to, the methods described in U.S. Pat. Nos. 7,456,273, 7,183,395, US2009/0093049, and WO2001/055371.

In some embodiments, a 5′UTR and/or 3′UTR comprising at least one TEE described herein can be incorporated in a monocistronic sequence such as, but not limited to, a vector system or a nucleic acid vector. As non-limiting examples, the vector systems and nucleic acid vectors can include those described in U.S. Pat. Nos. 7,456,273, 7,183,395, US2007/0048776, US2009/0093049, US2011/0124100, WO2007/025008, and WO2001/055371.

In some embodiments, a 5′UTR and/or 3′UTR of a polynucleotide of the invention comprises a TEE or portion thereof described herein. In some embodiments, the TEEs in the 3′UTR can be the same and/or different from the TEE located in the 5′UTR.

In some embodiments, a 5′UTR and/or 3′UTR of a polynucleotide of the invention can include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. In one embodiment, the 5′UTR of a polynucleotide of the invention can include 1-60, 1-55, 1-50, 1-45, 1-40, 1-35, 1-30, 1-25, 1-20, 1-15, 1-10, 9, 8, 7, 6, 5, 4, 3, 2, or 1 TEE sequences. The TEE sequences in the 5′UTR of the polynucleotide of the invention can be the same or different TEE sequences. A combination of different TEE sequences in the 5′UTR of the polynucleotide of the invention can include combinations in which more than one copy of any of the different TEE sequences are incorporated. The TEE sequences can be in a pattern such as ABABAB or AABBAABBAABB or ABCABCABC or variants thereof repeated one, two, three, or more than three times. In these patterns, each letter, A, B, or C represent a different TEE nucleotide sequence.

In some embodiments, the 5′UTR and/or 3′UTR comprises a spacer to separate two TEE sequences. As a non-limiting example, the spacer can be a 15 nucleotide spacer and/or other spacers known in the art. As another non-limiting example, the 5′UTR and/or 3′UTR comprises a TEE sequence-spacer module repeated at least once, at least twice, at least 3 times, at least 4 times, at least 5 times, at least 6 times, at least 7 times, at least 8 times, at least 9 times, at least 10 times, or more than 10 times in the 5′UTR and/or 3′UTR, respectively. In some embodiments, the 5′UTR and/or 3′UTR comprises a TEE sequence-spacer module repeated 1, 2, 3, 4, 5, 6, 7, 8, 9, or 10 times.

In some embodiments, the spacer separating two TEE sequences can include other sequences known in the art that can regulate the translation of the polynucleotide of the invention, e.g., miR sequences described herein (e.g., miR binding sites and miR seeds). As a non-limiting example, each spacer used to separate two TEE sequences can include a different miR sequence or component of a miR sequence (e.g., miR seed sequence).

In some embodiments, a polynucleotide of the invention comprises a miR and/or TEE sequence. In some embodiments, the incorporation of a miR sequence and/or a TEE sequence into a polynucleotide of the invention can change the shape of the stem loop region, which can increase and/or decrease translation. See e.g., Kedde et al, Nature Cell Biology 2010 12(10):1014-20, herein incorporated by reference in its entirety).

MicroRNA (miRNA) Binding Sites

Polynucleotides of the invention can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. In some embodiments, polynucleotides including such regulatory elements are referred to as including “sensor sequences”. Non-limiting examples of sensor sequences are described in U.S. Publication 2014/0200261, the contents of which are incorporated herein by reference in their entirety.

In some embodiments, a polynucleotide (e.g., a ribonucleic acid (RNA), e.g., a messenger RNA (mRNA)) of the invention comprises an open reading frame (ORF) encoding a polypeptide of interest and further comprises one or more miRNA binding site(s). Inclusion or incorporation of miRNA binding site(s) provides for regulation of polynucleotides of the invention, and in turn, of the polypeptides encoded therefrom, based on tissue-specific and/or cell-type specific expression of naturally-occurring miRNAs.

A miRNA, e.g., a natural-occurring miRNA, is a 19-25 nucleotide long noncoding RNA that binds to a polynucleotide and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh K K, Johnston W K, Garrett-Engele P, Lim L P, Bartel D P; Mol Cell. 2007 Jul. 6; 27(1):91-105. miRNA profiling of the target cells or tissues can be conducted to determine the presence or absence of miRNA in the cells or tissues. In some embodiments, a polynucleotide (e.g., a ribonucleic acid (RNA), e.g., a messenger RNA (mRNA)) of the invention comprises one or more microRNA binding sites, microRNA target sequences, microRNA complementary sequences, or microRNA seed complementary sequences. Such sequences can correspond to, e.g., have complementarity to, any known microRNA such as those taught in US Publication US2005/0261218 and US Publication US2005/0059005, the contents of each of which are incorporated herein by reference in their entirety.

As used herein, the term “microRNA (miRNA or miR) binding site” refers to a sequence within a polynucleotide, e.g., within a DNA or within an RNA transcript, including in the 5′UTR and/or 3′UTR, that has sufficient complementarity to all or a region of a miRNA to interact with, associate with or bind to the miRNA. In some embodiments, a polynucleotide of the invention comprising an ORF encoding a polypeptide of interest and further comprises one or more miRNA binding site(s). In exemplary embodiments, a 5′UTR and/or 3′UTR of the polynucleotide (e.g., a ribonucleic acid (RNA), e.g., a messenger RNA (mRNA)) comprises the one or more miRNA binding site(s).

A miRNA binding site having sufficient complementarity to a miRNA refers to a degree of complementarity sufficient to facilitate miRNA-mediated regulation of a polynucleotide, e.g., miRNA-mediated translational repression or degradation of the polynucleotide. In exemplary aspects of the invention, a miRNA binding site having sufficient complementarity to the miRNA refers to a degree of complementarity sufficient to facilitate miRNA-mediated degradation of the polynucleotide, e.g., miRNA-guided RNA-induced silencing complex (RISC)-mediated cleavage of mRNA. The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion less than 1, 2, 3, or 4 nucleotides of the full length of a naturally-occurring miRNA sequence. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with an miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with an miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the polynucleotide comprising the miRNA binding site. In other embodiments, the miRNA binding site has imperfect complementarity so that a RISC complex comprising the miRNA induces instability in the polynucleotide comprising the miRNA binding site. In another embodiment, the miRNA binding site has imperfect complementarity so that a RISC complex comprising the miRNA represses transcription of the polynucleotide comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a polynucleotide of the invention, the polynucleotide can be targeted for degradation or reduced translation, provided the miRNA in question is available. This can reduce off-target effects upon delivery of the polynucleotide. For example, if a polynucleotide of the invention is not intended to be delivered to a tissue or cell but ends up is said tissue or cell, then a miRNA abundant in the tissue or cell can inhibit the expression of the gene of interest if one or multiple binding sites of the miRNA are engineered into the 5′UTR and/or 3′UTR of the polynucleotide.

Conversely, miRNA binding sites can be removed from polynucleotide sequences in which they naturally occur in order to increase protein expression in specific tissues. For example, a binding site for a specific miRNA can be removed from a polynucleotide to improve protein expression in tissues or cells containing the miRNA.

In one embodiment, a polynucleotide of the invention can include at least one miRNA-binding site in the 5′UTR and/or 3′UTR in order to regulate cytotoxic or cytoprotective mRNA therapeutics to specific cells such as, but not limited to, normal and/or cancerous cells. In another embodiment, a polynucleotide of the invention can include two, three, four, five, six, seven, eight, nine, ten, or more miRNA-binding sites in the 5′-UTR and/or 3′-UTR in order to regulate cytotoxic or cytoprotective mRNA therapeutics to specific cells such as, but not limited to, normal and/or cancerous cells.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profilings in tissues and/or cells in development and/or disease. Identification of miRNAs, miRNA binding sites, and their expression patterns and role in biology have been reported (e.g., Bonauer et al, Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

miRNAs and miRNA binding sites can correspond to any known sequence, including non-limiting examples described in U.S. Publication Nos. 2014/0200261, 2005/0261218, and 2005/0059005, each of which are incorporated herein by reference in their entirety.

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Specifically, miRNAs are known to be differentially expressed in immune cells (also called hematopoietic cells), such as antigen presenting cells (APCs) (e.g., dendritic cells and macrophages), macrophages, monocytes, B lymphocytes, T lymphocytes, granulocytes, natural killer cells, etc. Immune cell specific miRNAs are involved in immunogenicity, autoimmunity, the immune-response to infection, inflammation, as well as unwanted immune response after gene therapy and tissue/organ transplantation. Immune cells specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a polynucleotide can be shut-off by adding miR-142 binding sites to the 3′-UTR of the polynucleotide, enabling more stable gene transfer in tissues and cells. miR-142 efficiently degrades exogenous polynucleotides in antigen presenting cells and suppresses cytotoxic elimination of transduced cells (e.g., Annoni A et al., blood, 2009, 114, 5152-5161; Brown B D, et al, Nat med. 2006, 12(5), 585-591; Brown B D, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing a miR-142 binding site into the 5′UTR and/or 3′UTR of a polynucleotide of the invention can selectively repress gene expression in antigen presenting cells through miR-142 mediated degradation, limiting antigen presentation in antigen presenting cells (e.g., dendritic cells) and thereby preventing antigen-mediated immune response after the delivery of the polynucleotide. The polynucleotide is then stably expressed in target tissues or cells without triggering cytotoxic elimination.

In one embodiment, binding sites for miRNAs that are known to be expressed in immune cells, in particular, antigen presenting cells, can be engineered into a polynucleotide of the invention to suppress the expression of the polynucleotide in antigen presenting cells through miRNA mediated RNA degradation, subduing the antigen-mediated immune response. Expression of the polynucleotide is maintained in non-immune cells where the immune cell specific miRNAs are not expressed. For example, in some embodiments, to prevent an immunogenic reaction against a liver specific protein, any miR-122 binding site can be removed and a miR-142 (and/or mirR-146) binding site can be engineered into the 5′UTR and/or 3′UTR of a polynucleotide of the invention.

In one embodiment, binding sites for miRNAs that are known to be expressed in liver cells can be engineered into a polynucleotide of the invention to suppress the expression of the polynucleotide in liver. For example, in some embodiments, to prevent expression of an antigen in liver, any liver specific miR binding site can be engineered into the 5′UTR and/or 3′UTR of a polynucleotide of the invention.

To further drive the selective degradation and suppression in APCs and macrophage, a polynucleotide of the invention can include a further negative regulatory element in the 5′UTR and/or 3′UTR, either alone or in combination with miR-142 and/or miR-146 binding sites. As a non-limiting example, the further negative regulatory element is a Constitutive Decay Element (CDE).

Immune cell specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1--3p, hsa-let-7f-2-5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p, miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p, miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11, 288, the content of each of which is incorporated herein by reference in its entirety.)

miRNAs that are known to be expressed in the liver include, but are not limited to, miR-107, miR-122-3p, miR-122-5p, miR-1228-3p, miR-1228-5p, miR-1249, miR-129-5p, miR-1303, miR-151a-3p, miR-151a-5p, miR-152, miR-194-3p, miR-194-5p, miR-199a-3p, miR-199a-5p, miR-199b-3p, miR-199b-5p, miR-296-5p, miR-557, miR-581, miR-939-3p, and miR-939-5p. MiRNA binding sites from any liver specific miRNA can be introduced to or removed from a polynucleotide of the invention to regulate expression of the polynucleotide in the liver. Liver specific miRNA binding sites can be engineered alone or further in combination with immune cell (e.g., APC) miRNA binding sites in a polynucleotide of the invention.

miRNAs that are known to be expressed in the lung include, but are not limited to, let-7a-2-3p, let-7a-3p, let-7a-5p, miR-126-3p, miR-126-5p, miR-127-3p, miR-127-5p, miR-130a-3p, miR-130a-5p, miR-130b-3p, miR-130b-5p, miR-133a, miR-133b, miR-134, miR-18a-3p, miR-18a-5p, miR-18b-3p, miR-18b-5p, miR-24-1-5p, miR-24-2-5p, miR-24-3p, miR-296-3p, miR-296-5p, miR-32-3p, miR-337-3p, miR-337-5p, miR-381-3p, and miR-381-5p. miRNA binding sites from any lung specific miRNA can be introduced to or removed from a polynucleotide of the invention to regulate expression of the polynucleotide in the lung. Lung specific miRNA binding sites can be engineered alone or further in combination with immune cell (e.g., APC) miRNA binding sites in a polynucleotide of the invention.

miRNAs that are known to be expressed in the heart include, but are not limited to, miR-1, miR-133a, miR-133b, miR-149-3p, miR-149-5p, miR-186-3p, miR-186-5p, miR-208a, miR-208b, miR-210, miR-296-3p, miR-320, miR-451a, miR-451b, miR-499a-3p, miR-499a-5p, miR-499b-3p, miR-499b-5p, miR-744-3p, miR-744-5p, miR-92b-3p, and miR-92b-5p. mMiRNA binding sites from any heart specific microRNA can be introduced to or removed from a polynucleotide of the invention to regulate expression of the polynucleotide in the heart. Heart specific miRNA binding sites can be engineered alone or further in combination with immune cell (e.g., APC) miRNA binding sites in a polynucleotide of the invention.

miRNAs that are known to be expressed in the nervous system include, but are not limited to, miR-124-5p, miR-125a-3p, miR-125a-5p, miR-125b-1-3p, miR-125b-2-3p, miR-125b-5p, miR-1271-3p, miR-1271-5p, miR-128, miR-132-5p, miR-135a-3p, miR-135a-5p, miR-135b-3p, miR-135b-5p, miR-137, miR-139-5p, miR-139-3p, miR-149-3p, miR-149-5p, miR-153, miR-181c-3p, miR-181c-5p, miR-183-3p, miR-183-5p, miR-190a, miR-190b, miR-212-3p, miR-212-5p, miR-219-1-3p, miR-219-2-3p, miR-23a-3p, miR-23a-5p, miR-30a-5p, miR-30b-3p, miR-30b-5p, miR-30c-1-3p, miR-30c-2-3p, miR-30c-5p, miR-30d-3p, miR-30d-5p, miR-329, miR-342-3p, miR-3665, miR-3666, miR-380-3p, miR-380-5p, miR-383, miR-410, miR-425-3p, miR-425-5p, miR-454-3p, miR-454-5p, miR-483, miR-510, miR-516a-3p, miR-548b-5p, miR-548c-5p, miR-571, miR-7-1-3p, miR-7-2-3p, miR-7-5p, miR-802, miR-922, miR-9-3p, and miR-9-5p. miRNAs enriched in the nervous system further include those specifically expressed in neurons, including, but not limited to, miR-132-3p, miR-132-3p, miR-148b-3p, miR-148b-5p, miR-151a-3p, miR-151a-5p, miR-212-3p, miR-212-5p, miR-320b, miR-320e, miR-323a-3p, miR-323a-5p, miR-324-5p, miR-325, miR-326, miR-328, miR-922 and those specifically expressed in glial cells, including, but not limited to, miR-1250, miR-219-1-3p, miR-219-2-3p, miR-219-5p, miR-23a-3p, miR-23a-5p, miR-3065-3p, miR-3065-5p, miR-30e-3p, miR-30e-5p, miR-32-5p, miR-338-5p, and miR-657. miRNA binding sites from any CNS specific miRNA can be introduced to or removed from a polynucleotide of the invention to regulate expression of the polynucleotide in the nervous system. Nervous system specific miRNA binding sites can be engineered alone or further in combination with immune cell (e.g., APC) miRNA binding sites in a polynucleotide of the invention.

miRNAs that are known to be expressed in the pancreas include, but are not limited to, miR-105-3p, miR-105-5p, miR-184, miR-195-3p, miR-195-5p, miR-196a-3p, miR-196a-5p, miR-214-3p, miR-214-5p, miR-216a-3p, miR-216a-5p, miR-30a-3p, miR-33a-3p, miR-33a-5p, miR-375, miR-7-1-3p, miR-7-2-3p, miR-493-3p, miR-493-5p, and miR-944. MiRNA binding sites from any pancreas specific miRNA can be introduced to or removed from a polynucleotide of the invention to regulate expression of the polynucleotide in the pancreas. Pancreas specific miRNA binding sites can be engineered alone or further in combination with immune cell (e.g. APC) miRNA binding sites in a polynucleotide of the invention.

miRNAs that are known to be expressed in the kidney include, but are not limited to, miR-122-3p, miR-145-5p, miR-17-5p, miR-192-3p, miR-192-5p, miR-194-3p, miR-194-5p, miR-20a-3p, miR-20a-5p, miR-204-3p, miR-204-5p, miR-210, miR-216a-3p, miR-216a-5p, miR-296-3p, miR-30a-3p, miR-30a-5p, miR-30b-3p, miR-30b-5p, miR-30c-1-3p, miR-30c-2-3p, miR30c-5p, miR-324-3p, miR-335-3p, miR-335-5p, miR-363-3p, miR-363-5p, and miR-562. miRNA binding sites from any kidney specific miRNA can be introduced to or removed from a polynucleotide of the invention to regulate expression of the polynucleotide in the kidney. Kidney specific miRNA binding sites can be engineered alone or further in combination with immune cell (e.g., APC) miRNA binding sites in a polynucleotide of the invention.

miRNAs that are known to be expressed in the muscle include, but are not limited to, let-7g-3p, let-7g-5p, miR-1, miR-1286, miR-133a, miR-133b, miR-140-3p, miR-143-3p, miR-143-5p, miR-145-3p, miR-145-5p, miR-188-3p, miR-188-5p, miR-206, miR-208a, miR-208b, miR-25-3p, and miR-25-5p. MiRNA binding sites from any muscle specific miRNA can be introduced to or removed from a polynucleotide of the invention to regulate expression of the polynucleotide in the muscle. Muscle specific miRNA binding sites can be engineered alone or further in combination with immune cell (e.g., APC) miRNA binding sites in a polynucleotide of the invention.

miRNAs are also differentially expressed in different types of cells, such as, but not limited to, endothelial cells, epithelial cells, and adipocytes.

miRNAs that are known to be expressed in endothelial cells include, but are not limited to, let-7b-3p, let-7b-5p, miR-100-3p, miR-100-5p, miR-101-3p, miR-101-5p, miR-126-3p, miR-126-5p, miR-1236-3p, miR-1236-5p, miR-130a-3p, miR-130a-5p, miR-17-5p, miR-17-3p, miR-18a-3p, miR-18a-5p, miR-19a-3p, miR-19a-5p, miR-19b-1-5p, miR-19b-2-5p, miR-19b-3p, miR-20a-3p, miR-20a-5p, miR-217, miR-210, miR-21-3p, miR-21-5p, miR-221-3p, miR-221-5p, miR-222-3p, miR-222-5p, miR-23a-3p, miR-23a-5p, miR-296-5p, miR-361-3p, miR-361-5p, miR-421, miR-424-3p, miR-424-5p, miR-513a-5p, miR-92a-1-5p, miR-92a-2-5p, miR-92a-3p, miR-92b-3p, and miR-92b-5p. Many novel miRNAs are discovered in endothelial cells from deep-sequencing analysis (e.g., Voellenkle C et al., RNA, 2012, 18, 472-484, herein incorporated by reference in its entirety). miRNA binding sites from any endothelial cell specific miRNA can be introduced to or removed from a polynucleotide of the invention to regulate expression of the polynucleotide in the endothelial cells.

miRNAs that are known to be expressed in epithelial cells include, but are not limited to, let-7b-3p, let-7b-5p, miR-1246, miR-200a-3p, miR-200a-5p, miR-200b-3p, miR-200b-5p, miR-200c-3p, miR-200c-5p, miR-338-3p, miR-429, miR-451a, miR-451b, miR-494, miR-802 and miR-34a, miR-34b-5p, miR-34c-5p, miR-449a, miR-449b-3p, miR-449b-5p specific in respiratory ciliated epithelial cells, let-7 family, miR-133a, miR-133b, miR-126 specific in lung epithelial cells, miR-382-3p, miR-382-5p specific in renal epithelial cells, and miR-762 specific in corneal epithelial cells. miRNA binding sites from any epithelial cell specific miRNA can be introduced to or removed from a polynucleotide of the invention to regulate expression of the polynucleotide in the epithelial cells.

In addition, a large group of miRNAs are enriched in embryonic stem cells, controlling stem cell self-renewal as well as the development and/or differentiation of various cell lineages, such as neural cells, cardiac, hematopoietic cells, skin cells, osteogenic cells and muscle cells (e.g., Kuppusamy K T et al., Curr. Mol Med, 2013, 13(5), 757-764; Vidigal J A and Ventura A, Semin Cancer Biol. 2012, 22(5-6), 428-436; Goff L A et al., PLoS One, 2009, 4:e7192; Morin R D et al, Genome Res, 2008,18, 610-621; Yoo J K et al, Stem Cells Dev. 2012, 21(11), 2049-2057, each of which is herein incorporated by reference in its entirety). MiRNAs abundant in embryonic stem cells include, but are not limited to, let-7a-2-3p, let-a-3p, let-7a-5p, let7d-3p, let-7d-5p, miR-103a-2-3p, miR-103a-5p, miR-106b-3p, miR-106b-5p, miR-1246, miR-1275, miR-138-1-3p, miR-138-2-3p, miR-138-5p, miR-154-3p, miR-154-5p, miR-200c-3p, miR-200c-5p, miR-290, miR-301a-3p, miR-301a-5p, miR-302a-3p, miR-302a-5p, miR-302b-3p, miR-302b-5p, miR-302c-3p, miR-302c-5p, miR-302d-3p, miR-302d-5p, miR-302e, miR-367-3p, miR-367-5p, miR-369-3p, miR-369-5p, miR-370, miR-371, miR-373, miR-380-5p, miR-423-3p, miR-423-5p, miR-486-5p, miR-520c-3p, miR-548e, miR-548f, miR-548g-3p, miR-548g-5p, miR-548i, miR-548k, miR-5481, miR-548m, miR-548n, miR-548o-3p, miR-548o-5p, miR-548p, miR-664a-3p, miR-664a-5p, miR-664b-3p, miR-664b-5p, miR-766-3p, miR-766-5p, miR-885-3p, miR-885-5p, miR-93-3p, miR-93-5p, miR-941,miR-96-3p, miR-96-5p, miR-99b-3p and miR-99b-5p. Many predicted novel miRNAs are discovered by deep sequencing in human embryonic stem cells (e.g., Morin R D et al., Genome Res, 2008,18, 610-621; Goff L A et al., PLoS One, 2009, 4:e7192; Bar M et al, Stem cells, 2008, 26, 2496-2505, the content of each of which is incorporated herein by reference in its entirety).

Many miRNA expression studies are conducted to profile the differential expression of miRNAs in various cancer cells/tissues and other diseases. Some miRNAs are abnormally over-expressed in certain cancer cells and others are under-expressed. For example, miRNAs are differentially expressed in cancer cells (WO2008/154098, US2013/0059015, US2013/0042333, WO2011/157294); cancer stem cells (US2012/0053224); pancreatic cancers and diseases (US2009/0131348, US2011/0171646, US2010/0286232, U.S. Pat. No. 8,389,210); asthma and inflammation (U.S. Pat. No. 8,415,096); prostate cancer (US2013/0053264); hepatocellular carcinoma (WO2012/151212, US2012/0329672, WO2008/054828, U.S. Pat. No. 8,252,538); lung cancer cells (WO2011/076143, WO2013/033640, WO2009/070653, US2010/0323357); cutaneous T cell lymphoma (WO2013/011378); colorectal cancer cells (WO2011/0281756, WO2011/076142); cancer positive lymph nodes (WO2009/100430, US2009/0263803); nasopharyngeal carcinoma (EP2112235); chronic obstructive pulmonary disease (US2012/0264626, US2013/0053263); thyroid cancer (WO2013/066678); ovarian cancer cells (US2012/0309645, WO2011/095623); breast cancer cells (WO2008/154098, WO2007/081740, US2012/0214699), leukemia and lymphoma (WO2008/073915, US2009/0092974, US2012/0316081, US2012/0283310, WO2010/018563, the content of each of which is incorporated herein by reference in its entirety.)

As a non-limiting example, miRNA binding sites for miRNAs that are over-expressed in certain cancer and/or tumor cells can be removed from the 3′UTR of a polynucleotide of the invention, restoring the expression suppressed by the over-expressed miRNAs in cancer cells, thus ameliorating the corresponsive biological function, for instance, transcription stimulation and/or repression, cell cycle arrest, apoptosis and cell death. Normal cells and tissues, wherein miRNAs expression is not up-regulated, will remain unaffected.

miRNA can also regulate complex biological processes such as angiogenesis (e.g., miR-132) (Anand and Cheresh Curr Opin Hematol 2011 18:171-176). In the polynucleotides of the invention, miRNA binding sites that are involved in such processes can be removed or introduced, in order to tailor the expression of the polynucleotides to biologically relevant cell types or relevant biological processes. In this context, the polynucleotides of the invention are defined as auxotrophic polynucleotides.

In some embodiments, a polynucleotide of the invention comprises a miRNA binding site, wherein the miRNA binding site comprises one or more nucleotide sequences selected from TABLE 1 or described elsewhere herein, including one or more copies of any one or more of the miRNA binding site sequences. In some embodiments, a polynucleotide of the invention further comprises at least one, two, three, four, five, six, seven, eight, nine, ten, or more of the same or different miRNA binding sites selected from TABLE 1 or described elsewhere herein, including any combination thereof. In some embodiments, the miRNA binding site binds to miR-142 or is complementary to miR-142. In some embodiments, the miR-142 comprises SEQ ID NO: 303. In some embodiments, the miRNA binding site binds to miR-142-3p or miR-142-5p. In some embodiments, the miR-142-3p binding site comprises SEQ ID NO: 305. In some embodiments, the miR-142-5p binding site comprises SEQ ID NO: 307. In some embodiments, the miRNA binding site comprises a nucleotide sequence at least 80%, at least 85%, at least 90%, at least 95%, or 100% identical to SEQ ID NOs: 305 or 307.

TABLE 1
miR-142 and alternative miR-142 binding sites

| SEQ ID NO. | Description | Sequence |
|---|---|---|
| 303 | miR-142 | GACAGUGCAGUCACCCAUAAAGUAGAAAGCACUACUA |
| | | ACAGCACUGGAGGGUGUAGUGUUUCCUACUUUAUGGA |
| | | UGAGUGUACUGUG |
| 304 | miR-142-3p | UGUAGUGUUUCCUACUUUAUGGA |
| 305 | miR-142-3p binding | UCCAUAAAGUAGGAAACACUACA |
| | site | |
| 306 | miR-142-5p | CAUAAAGUAGAAAGCACUACU |
| 307 | miR-142-5p binding | AGUAGUGCUUUCUACUUUAUG |
| | site | |

In some embodiments, a miRNA binding site is inserted in the polynucleotide of the invention in any position of the polynucleotide (e.g., the 5′UTR and/or 3′UTR). In some embodiments, the 5′UTR comprises a miRNA binding site. In some embodiments, the 3′UTR comprises a miRNA binding site. In some embodiments, the 5′UTR and the 3′UTR comprise a miRNA binding site. The insertion site in the polynucleotide can be anywhere in the polynucleotide as long as the insertion of the miRNA binding site in the polynucleotide does not interfere with the translation of a functional polypeptide in the absence of the corresponding miRNA; and in the presence of the miRNA, the insertion of the miRNA binding site in the polynucleotide and the binding of the miRNA binding site to the corresponding miRNA are capable of degrading the polynucleotide or preventing the translation of the polynucleotide.

In some embodiments, a miRNA binding site is inserted in at least about 30 nucleotides downstream from the stop codon of an ORF in a polynucleotide of the invention comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the invention. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a polynucleotide of the invention.

miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′UTR of the same sequence type.

In one embodiment, other regulatory elements and/or structural elements of the 5′UTR can influence miRNA mediated gene regulation. One example of a regulatory element and/or structural element is a structured IRES (Internal Ribosome Entry Site) in the 5′UTR, which is necessary for the binding of translational elongation factors to initiate protein translation. EIF4A2 binding to this secondarily structured element in the 5′-UTR is necessary for miRNA mediated gene expression (Meijer H A et at, Science, 2013, 340, 82-85, herein incorporated by reference in its entirety). The polynucleotides of the invention can further include this structured 5′UTR in order to enhance microRNA mediated gene regulation.

At least one miRNA binding site can be engineered into the 3′UTR of a polynucleotide of the invention. In this context, at least two, at least three, at least four, at least five, at least six, at least seven, at least eight, at least nine, at least ten, or more miRNA binding sites can be engineered into a 3′UTR of a polynucleotide of the invention. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a polynucleotide of the invention. In one embodiment, miRNA binding sites incorporated into a polynucleotide of the invention can be the same or can be different miRNA sites. A combination of different miRNA binding sites incorporated into a polynucleotide of the invention can include combinations in which more than one copy of any of the different miRNA sites are incorporated. In another embodiment, miRNA binding sites incorporated into a polynucleotide of the invention can target the same or different tissues in the body. As a non-limiting example, through the introduction of tissue-, cell-type-, or disease-specific miRNA binding sites in the 3′-UTR of a polynucleotide of the invention, the degree of expression in specific cell types (e.g., hepatocytes, myeloid cells, endothelial cells, cancer cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′UTR in a polynucleotide of the invention. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′UTR. As yet another non-limiting example, a miRNA binding site can be engineered near the 5′ terminus of the 3′UTR and near the 3′ terminus of the 3′UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

In one embodiment, a polynucleotide of the invention can be engineered to include more than one miRNA site expressed in different tissues or different cell types of a subject. As a non-limiting example, a polynucleotide of the invention can be engineered to include miR-192 and miR-122 to regulate expression of the polynucleotide in the liver and kidneys of a subject. In another embodiment, a polynucleotide of the invention can be engineered to include more than one miRNA site for the same tissue.

In some embodiments, the therapeutic window and or differential expression associated with the polypeptide encoded by a polynucleotide of the invention can be altered with a miRNA binding site. For example, a polynucleotide encoding a polypeptide that provides a death signal can be designed to be more highly expressed in cancer cells by virtue of the miRNA signature of those cells. Where a cancer cell expresses a lower level of a particular miRNA, the polynucleotide encoding the binding site for that miRNA (or miRNAs) would be more highly expressed. Hence, the polypeptide that provides a death signal triggers or induces cell death in the cancer cell. Neighboring noncancer cells, harboring a higher expression of the same miRNA would be less affected by the encoded death signal as the polynucleotide would be expressed at a lower level due to the effects of the miRNA binding to the binding site or “sensor” encoded in the 3′UTR. Conversely, cell survival or cytoprotective signals can be delivered to tissues containing cancer and non-cancerous cells where a miRNA has a higher expression in the cancer cells—the result being a lower survival signal to the cancer cell and a larger survival signal to the normal cell. Multiple polynucleotides can be designed and administered having different signals based on the use of miRNA binding sites as described herein.

In some embodiments, the expression of a polynucleotide of the invention can be controlled by incorporating at least one miR binding site or sensor sequence in the polynucleotide and formulating the polynucleotide for administration. As a non-limiting example, a polynucleotide of the invention can be targeted to a tissue or cell by incorporating a miRNA binding site and formulating the polynucleotide in a lipid nanoparticle comprising a ionizable lipid (e.g., a cationic lipid), including any of the lipids described herein.

A polynucleotide of the invention can be engineered for more targeted expression in specific tissues, cell types, or biological conditions based on the expression patterns of miRNAs in the different tissues, cell types, or biological conditions. Through introduction of tissue-specific miRNA binding sites, a polynucleotide of the invention can be designed for optimal protein expression in a tissue or cell, or in the context of a biological condition.

In some embodiments, a polynucleotide of the invention can be designed to incorporate miRNA binding sites that either have 100% identity to known miRNA seed sequences or have less than 100% identity to miRNA seed sequences. In some embodiments, a polynucleotide of the invention can be designed to incorporate miRNA binding sites that have at least: 60%, 65%, 70%, 75%, 80%, 85%, 90%, 95%, 96%, 97%, 98%, or 99% identity to known miRNA seed sequences. The miRNA seed sequence can be partially mutated to decrease miRNA binding affinity and as such result in reduced downmodulation of the polynucleotide. In essence, the degree of match or mis-match between the miRNA binding site and the miRNA seed can act as a rheostat to more finely tune the ability of the miRNA to modulate protein expression. In addition, mutation in the non-seed region of a miRNA binding site can also impact the ability of a miRNA to modulate protein expression.

In one embodiment, a miRNA sequence can be incorporated into the loop of a stem loop.

In another embodiment, a miRNA seed sequence can be incorporated in the loop of a stem loop and a miRNA binding site can be incorporated into the 5′ or 3′ stem of the stem loop.

In one embodiment, a translation enhancer element (TEE) can be incorporated on the 5′end of the stem of a stem loop and a miRNA seed can be incorporated into the stem of the stem loop. In another embodiment, a TEE can be incorporated on the 5′ end of the stem of a stem loop, a miRNA seed can be incorporated into the stem of the stem loop and a miRNA binding site can be incorporated into the 3′ end of the stem or the sequence after the stem loop. The miRNA seed and the miRNA binding site can be for the same and/or different miRNA sequences.

In one embodiment, the incorporation of a miRNA sequence and/or a TEE sequence changes the shape of the stem loop region which can increase and/or decrease translation. (see e.g, Kedde et al., “A Pumilio-induced RNA structure switch in p27-3′UTR controls miR-221 and miR-22 accessibility.” Nature Cell Biology. 2010, incorporated herein by reference in its entirety).

In one embodiment, the 5′-UTR of a polynucleotide of the invention can comprise at least one miRNA sequence. The miRNA sequence can be, but is not limited to, a 19 or 22 nucleotide sequence and/or a miRNA sequence without the seed.

In one embodiment the miRNA sequence in the 5′UTR can be used to stabilize a polynucleotide of the invention described herein.

In another embodiment, a miRNA sequence in the 5′UTR of a polynucleotide of the invention can be used to decrease the accessibility of the site of translation initiation such as, but not limited to a start codon. See, e.g., Matsuda et al, PLoS One. 2010 11(5):e15057; incorporated herein by reference in its entirety, which used antisense locked nucleic acid (LNA) oligonucleotides and exon-junction complexes (EJCs) around a start codon (−4 to +37 where the A of the AUG codons is +1) in order to decrease the accessibility to the first start codon (AUG). Matsuda showed that altering the sequence around the start codon with an LNA or EJC affected the efficiency, length and structural stability of a polynucleotide. A polynucleotide of the invention can comprise a miRNA sequence, instead of the LNA or EJC sequence described by Matsuda et al, near the site of translation initiation in order to decrease the accessibility to the site of translation initiation. The site of translation initiation can be prior to, after or within the miRNA sequence. As a non-limiting example, the site of translation initiation can be located within a miRNA sequence such as a seed sequence or binding site. As another non-limiting example, the site of translation initiation can be located within a miR-122 sequence such as the seed sequence or the mir-122 binding site.

In some embodiments, a polynucleotide of the invention can include at least one miRNA in order to dampen the antigen presentation by antigen presenting cells. The miRNA can be the complete miRNA sequence, the miRNA seed sequence, the miRNA sequence without the seed, or a combination thereof. As a non-limiting example, a miRNA incorporated into a polynucleotide of the invention can be specific to the hematopoietic system. As another non-limiting example, a miRNA incorporated into a polynucleotide of the invention to dampen antigen presentation is miR-142-3p.

In some embodiments, a polynucleotide of the invention can include at least one miRNA in order to dampen expression of the encoded polypeptide in a tissue or cell of interest. As a non-limiting example, a polynucleotide of the invention can include at least one miR-122 binding site in order to dampen expression of an encoded polypeptide of interest in the liver. As another non-limiting example a polynucleotide of the invention can include at least one miR-142-3p binding site, miR-142-3p seed sequence, miR-142-3p binding site without the seed, miR-142-5p binding site, miR-142-5p seed sequence, miR-142-5p binding site without the seed, miR-146 binding site, miR-146 seed sequence and/or miR-146 binding site without the seed sequence.

In some embodiments, a polynucleotide of the invention can comprise at least one miRNA binding site in the 3′UTR in order to selectively degrade mRNA therapeutics in the immune cells to subdue unwanted immunogenic reactions caused by therapeutic delivery. As a non-limiting example, the miRNA binding site can make a polynucleotide of the invention more unstable in antigen presenting cells. Non-limiting examples of these miRNAs include mir-142-5p, mir-142-3p, mir-146a-5p, and mir-146-3p.

In one embodiment, a polynucleotide of the invention comprises at least one miRNA sequence in a region of the polynucleotide that can interact with a RNA binding protein.

In some embodiments, the polynucleotide of the invention (e.g., a RNA, e.g., an mRNA) comprising (i) a sequence-optimized nucleotide sequence (e.g., an ORF) encoding one or more wild type epitope antigens and (ii) a miRNA binding site (e.g., a miRNA binding site that binds to miR-142).

In some embodiments, the polynucleotide of the invention comprises a uracil-modified sequence encoding one or more cancer epitope polypeptides disclosed herein and a miRNA binding site disclosed herein, e.g., a miRNA binding site that binds to miR-142. In some embodiments, the uracil-modified sequence encoding one or more cancer epitope polypeptides comprises at least one chemically modified nucleobase, e.g., 5-methoxyuracil. In some embodiments, at least 95% of a type of nucleobase (e.g., uricil) in a uracil-modified sequence encoding one or more cancer epitope polypeptides of the invention are modified nucleobases. In some embodiments, at least 95% of uricil in a uracil-modified sequence encoding one or more cancer epitope polypeptides is 5-methoxyuridine. In some embodiments, the polynucleotide comprising a nucleotide sequence encoding one or more cancer epitope polypeptides disclosed herein and a miRNA binding site is formulated with a delivery agent, e.g., a LNP comprising, for instance, a lipid having the Formula (I), (IA), (II), (IIa), (IIb), (IIc), (IId) or (IIe), e.g., any of Compounds 1-232.

3′ UTR and the A URich Elements

In certain embodiments, a polynucleotide of the present invention (e.g., a polynucleotide comprising a nucleotide sequence encoding a cancer antigen epitope of the invention) further comprises a 3′ UTR. In certain embodiments, a polynucleotide of the present invention (e.g., a polynucleotide comprising a nucleotide sequence encoding an activating oncogene mutation peptide of the invention) further comprises a 3′ UTR.

3′-UTR is the section of mRNA that immediately follows the translation termination codon and often contains regulatory regions that post-transcriptionally influence gene expression. Regulatory regions within the 3′-UTR can influence polyadenylation, translation efficiency, localization, and stability of the mRNA. In one embodiment, the 3′-UTR useful for the invention comprises a binding site for regulatory proteins or microRNAs. In some embodiments, the 3′-UTR has a silencer region, which binds to repressor proteins and inhibits the expression of the mRNA. In other embodiments, the 3′-UTR comprises an AU-rich element. Proteins bind AREs to affect the stability or decay rate of transcripts in a localized manner or affect translation initiation. In other embodiments, the 3′-UTR comprises the sequence AAUAAA that directs addition of several hundred adenine residues called the poly(A) tail to the end of the mRNA transcript.

Natural or wild type 3′ UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides of the invention. When engineering specific polynucleotides, one or more copies of an ARE can be introduced to make polynucleotides of the invention less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using polynucleotides of the invention and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

Regions Having a 5′ Cap

The invention also includes a polynucleotide that comprises both a 5′ Cap and a polynucleotide of the present invention (e.g., a polynucleotide comprising a nucleotide sequence encoding a cancer antigen epitope such as an activating oncogene mutation peptide).

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or anteterminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

In some embodiments, the polynucleotides of the present invention (e.g., a polynucleotide comprising a nucleotide sequence encoding a cancer antigen epitope) incorporate a cap moiety.

In some embodiments, polynucleotides of the present invention (e.g., a polynucleotide comprising a nucleotide sequence encoding a cancer antigen epitope such as an activating oncogene mutation peptide) comprise a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, Mass.) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which can equivalently be designated 3′ O-Me-m7G(5′)ppp(5′)G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

In some embodiments, the cap is a dinucleotide cap analog. As a non-limiting example, the dinucleotide cap analog can be modified at different phosphate positions with a boranophosphate group or a phophoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dicucleotide form of a cap analog known in the art and/or described herein. Non-limiting examples of a N7-(4-chlorophenoxyethyl) substituted dicucleotide form of a cap analog include a N7-(4-chlorophenoxyethyl)-G(5′)ppp(5′)G and a N7-(4-chlorophenoxyethyl)-m3′-OG(5′)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present invention is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

Polynucleotides of the invention (e.g., a polynucleotide comprising a nucleotide sequence encoding a cancer antigen epitope) can also be capped post-manufacture (whether IVT or chemical synthesis), using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those that, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)NlmpNp (cap 1), and 7mG(5′)-ppp(5′)NlmpN2mp (cap 2).

As a non-limiting example, capping chimeric polynucleotides post-manufacture can be more efficient as nearly 100% of the chimeric polynucleotides can be capped. This is in contrast to ˜80% when a cap analog is linked to a chimeric polynucleotide in the course of an in vitro transcription reaction.

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

Poly-A Tails

In some embodiments, the polynucleotides of the present disclosure (e.g., a polynucleotide comprising a nucleotide sequence encoding a cancer antigen epitope such as an activating oncogene mutation peptide) further comprise a poly-A tail. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a polynucleotide such as an mRNA molecule in order to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long.

PolyA tails can also be added after the construct is exported from the nucleus.

According to the present invention, terminal groups on the poly A tail can be incorporated for stabilization. Polynucleotides of the present invention can include des-3′ hydroxyl tails. They can also include structural moieties or 2′-Omethyl modifications as taught by Junjie Li, et at (Current Biology, Vol. 15, 1501-1507, Aug. 23, 2005, the contents of which are incorporated herein by reference in its entirety).

The polynucleotides of the present invention can be designed to encode transcripts with alternative polyA tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi:10.1038/nrm3645) the contents of which are incorporated herein by reference in its entirety.

Unique poly-A tail lengths provide certain advantages to the polynucleotides of the present invention. Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present invention are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone.

Start Codon Region

The invention also includes a polynucleotide that comprises both a start codon region and the polynucleotide described herein (e.g., a polynucleotide comprising a nucleotide sequence encoding a cancer antigen epitope such as an activating oncogene mutation peptide). In some embodiments, the polynucleotides of the present invention can have regions that are analogous to or function like a start codon region.

In some embodiments, the translation of a polynucleotide can initiate on a codon that is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG (see Touriol et at Biology of the Cell 95 (2003) 169-178 and Matsuda and Mauro PLoS ONE, 2010 5:11; the contents of each of which are herein incorporated by reference in its entirety).

As a non-limiting example, the translation of a polynucleotide begins on the alternative start codon ACG. As another non-limiting example, polynucleotide translation begins on the alternative start codon CTG or CUG. As yet another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See, e.g., Matsuda and Mauro PLoS ONE, 2010 5:11; the contents of which are herein incorporated by reference in its entirety). Masking any of the nucleotides flanking a codon that initiates translation can be used to alter the position of translation initiation, translation efficiency, length and/or structure of a polynucleotide.

In some embodiments, a masking agent can be used near the start codon or alternative start codon in order to mask or hide the codon to reduce the probability of translation initiation at the masked start codon or alternative start codon. Non-limiting examples of masking agents include antisense locked nucleic acids (LNA) polynucleotides and exon-junction complexes (EJCs) (See, e.g., Matsuda and Mauro describing masking agents LNA polynucleotides and EJCs (PLoS ONE, 2010 5:11); the contents of which are herein incorporated by reference in its entirety).

In another embodiment, a masking agent can be used to mask a start codon of a polynucleotide in order to increase the likelihood that translation will initiate on an alternative start codon. In some embodiments, a masking agent can be used to mask a first start codon or alternative start codon in order to increase the chance that translation will initiate on a start codon or alternative start codon downstream to the masked start codon or alternative start codon.

In some embodiments, a start codon or alternative start codon can be located within a perfect complement for a miR binding site. The perfect complement of a miR binding site can help control the translation, length and/or structure of the polynucleotide similar to a masking agent. As a non-limiting example, the start codon or alternative start codon can be located in the middle of a perfect complement for a miRNA binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide can be removed from the polynucleotide sequence in order to have the translation of the polynucleotide begin on a codon that is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence in order to have translation initiate on a downstream start codon or alternative start codon. The polynucleotide sequence where the start codon was removed can further comprise at least one masking agent for the downstream start codon and/or alternative start codons in order to control or attempt to control the initiation of translation, the length of the polynucleotide and/or the structure of the polynucleotide.

Stop Codon Region

The invention also includes a polynucleotide that comprises both a stop codon region and the polynucleotide described herein (e.g., a polynucleotide comprising a nucleotide sequence encoding a cancer antigen epitope such as an activating oncogene mutation peptide). In some embodiments, the polynucleotides of the present invention can include at least two stop codons before the 3′ untranslated region (UTR). The stop codon can be selected from TGA, TAA and TAG in the case of DNA, or from UGA, UAA and UAG in the case of RNA. In some embodiments, the polynucleotides of the present invention include the stop codon TGA in the case or DNA, or the stop codon UGA in the case of RNA, and one additional stop codon. In a further embodiment the addition stop codon can be TAA or UAA. In another embodiment, the polynucleotides of the present invention include three consecutive stop codons, four stop codons, or more.

Insertions and Substitutions

The invention also includes a polynucleotide of the present disclosure that further comprises insertions and/or substitutions.

In some embodiments, the 5′UTR of the polynucleotide can be replaced by the insertion of at least one region and/or string of nucleosides of the same base. The region and/or string of nucleotides can include, but is not limited to, at least 3, at least 4, at least 5, at least 6, at least 7 or at least 8 nucleotides and the nucleotides can be natural and/or unnatural. As a non-limiting example, the group of nucleotides can include 5-8 adenine, cytosine, thymine, a string of any of the other nucleotides disclosed herein and/or combinations thereof.

In some embodiments, the 5′UTR of the polynucleotide can be replaced by the insertion of at least two regions and/or strings of nucleotides of two different bases such as, but not limited to, adenine, cytosine, thymine, any of the other nucleotides disclosed herein and/or combinations thereof. For example, the 5′UTR can be replaced by inserting 5-8 adenine bases followed by the insertion of 5-8 cytosine bases. In another example, the 5′UTR can be replaced by inserting 5-8 cytosine bases followed by the insertion of 5-8 adenine bases.

In some embodiments, the polynucleotide can include at least one substitution and/or insertion downstream of the transcription start site that can be recognized by an RNA polymerase. As a non-limiting example, at least one substitution and/or insertion can occur downstream of the transcription start site by substituting at least one nucleic acid in the region just downstream of the transcription start site (such as, but not limited to, +1 to +6). Changes to region of nucleotides just downstream of the transcription start site can affect initiation rates, increase apparent nucleotide triphosphate (NTP) reaction constant values, and increase the dissociation of short transcripts from the transcription complex curing initial transcription (Brieba et al, Biochemistry (2002) 41: 5144-5149; herein incorporated by reference in its entirety). The modification, substitution and/or insertion of at least one nucleoside can cause a silent mutation of the sequence or can cause a mutation in the amino acid sequence.

In some embodiments, the polynucleotide can include the substitution of at least 1, at least 2, at least 3, at least 4, at least 5, at least 6, at least 7, at least 8, at least 9, at least 10, at least 11, at least 12 or at least 13 guanine bases downstream of the transcription start site.

In some embodiments, the polynucleotide can include the substitution of at least 1, at least 2, at least 3, at least 4, at least 5 or at least 6 guanine bases in the region just downstream of the transcription start site. As a non-limiting example, if the nucleotides in the region are GGGAGA, the guanine bases can be substituted by at least 1, at least 2, at least 3 or at least 4 adenine nucleotides. In another non-limiting example, if the nucleotides in the region are GGGAGA the guanine bases can be substituted by at least 1, at least 2, at least 3 or at least 4 cytosine bases. In another non-limiting example, if the nucleotides in the region are GGGAGA the guanine bases can be substituted by at least 1, at least 2, at least 3 or at least 4 thymine, and/or any of the nucleotides described herein.

In some embodiments, the polynucleotide can include at least one substitution and/or insertion upstream of the start codon. For the purpose of clarity, one of skill in the art would appreciate that the start codon is the first codon of the protein coding region whereas the transcription start site is the site where transcription begins. The polynucleotide can include, but is not limited to, at least 1, at least 2, at least 3, at least 4, at least 5, at least 6, at least 7 or at least 8 substitutions and/or insertions of nucleotide bases. The nucleotide bases can be inserted or substituted at 1, at least 1, at least 2, at least 3, at least 4 or at least 5 locations upstream of the start codon. The nucleotides inserted and/or substituted can be the same base (e.g., all A or all C or all T or all G), two different bases (e.g., A and C, A and T, or C and T), three different bases (e.g., A, C and T or A, C and T) or at least four different bases.

As a non-limiting example, the guanine base upstream of the coding region in the polynucleotide can be substituted with adenine, cytosine, thymine, or any of the nucleotides described herein. In another non-limiting example, the substitution of guanine bases in the polynucleotide can be designed so as to leave one guanine base in the region downstream of the transcription start site and before the start codon (see Esvelt et al. Nature (2011) 472(7344):499-503; the contents of which is herein incorporated by reference in its entirety). As a non-limiting example, at least 5 nucleotides can be inserted at 1 location downstream of the transcription start site but upstream of the start codon and the at least 5 nucleotides can be the same base type.

According to the present disclosure, two regions or parts of a chimeric polynucleotide may be joined or ligated, for example, using triphosphate chemistry. In some embodiments, a first region or part of 100 nucleotides or less is chemically synthesized with a 5′-monophosphate and terminal 3′-desOH or blocked OH. If the region is longer than 80 nucleotides, it may be synthesized as two or more strands that will subsequently be chemically linked by ligation. If the first region or part is synthesized as a non-positionally modified region or part using IVT, conversion to the 5′-monophosphate with subsequent capping of the 3′-terminus may follow. Monophosphate protecting groups may be selected from any of those known in the art. A second region or part of the chimeric polynucleotide may be synthesized using either chemical synthesis or IVT methods, e.g., as described herein. IVT methods may include use of an RNA polymerase that can utilize a primer with a modified cap. Alternatively, a cap may be chemically synthesized and coupled to the IVT region or part.

It is noted that for ligation methods, ligation with DNA T4 ligase followed by DNAse treatment (to eliminate the DNA splint required for DNA T4 Ligase activity) should readily prevent the undesirable formation of concatenation products.

The entire chimeric polynucleotide need not be manufactured with a phosphate-sugar backbone. If one of the regions or parts encodes a polypeptide, then it is preferable that such region or part comprise a phosphate-sugar backbone.

Ligation may be performed using any appropriate technique, such as enzymatic ligation, click chemistry, orthoclick chemistry, solulink, or other bioconjugate chemistries known to those in the art. In some embodiments, the ligation is directed by a complementary oligonucleotide splint. In some embodiments, the ligation is performed without a complementary oligonucleotide splint.

In other aspects, the invention relates to kits for preparing an mRNA cancer vaccine by IVT methods. In personalized cancer vaccines, it is important to identify patient specific mutations and vaccinate the patient with one or more neoepitopes. In such vaccines, the antigen(s) encoded by the ORFs of an mRNA will be specific to the patient. The 5′- and 3′-ends of RNAs encoding the antigen(s) may be more broadly applicable, as they include untranslated regions and stabilizing regions that are common to many RNAs. Among other things, the present disclosure provides kits that include one or parts of a chimeric polynucleotide, such as one or more 5′- and/or 3′-regions of RNA, which may be combined with an ORF encoding a patient-specific epitope. For example, a kit may include a polynucleotide containing one or more of a 5′-ORF, a 3′-ORF, and a poly(A) tail. In some embodiments, each polynucleotide component is in an individual container. In other embodiments, more than one polynucleotide component is present together in a single container. In some embodiments, the kit includes a ligase enzyme. In some embodiments, provided kits include instructions for use. In some embodiments, the instructions include an instruction to ligate the epitope encoding ORF to one or more other components from the kit, e.g., 5′-ORF, a 3′-ORF, and/or a poly(A) tail.

Methods for generating personalized cancer vaccines according to the invention involve identification of mutations using techniques such as deep nucleic acid or protein sequencing methods as described herein of tissue samples. In some embodiments an initial identification of mutations in a patient's transcriptome is performed. The data from the patient's transcriptome is compared with sequence information from the patients exome in order to identify patient specific and tumor specific mutations that are expressed. The comparison produces a dataset of putative neoepitopes, referred to as a mutanome. The mutanome may include approximately 100-10,000 candidate mutations per patients. The mutanome is subject to a data probing analysis using a set of inquiries or algorithms to identify an optimal mutation set for generation of a neoantigen vaccine. In some embodiments an mRNA neoantigen vaccine is designed and manufactured. The patient is then treated with the vaccine.

The neoantigen vaccine may be a polycistronic vaccine including multiple neoepitopes or one or more single RNA vaccines or a combination thereof.

In some embodiments the entire method from the initiation of the mutation identification process to the start of patient treatment is achieved in less than 2 months. In other embodiments the whole process is achieved in 7 weeks or less, 6 weeks or less, 5 weeks or less, 4 weeks or less, 3 weeks or less, 2 weeks or less or less than 1 week. In some embodiments the whole method is performed in less than 30 days.

The mutation identification process may involve both transcriptome and exome analysis or only transcriptome or exome analysis. In some embodiments transcriptome analysis is performed first and exome analysis is performed second. The analysis is performed on a biological or tissue sample. In some embodiments a biological or tissue sample is a blood or serum sample. In other embodiments the sample is a tissue bank sample or EBV transformation of B-cells.

It has been recognized and appreciated that, by analyzing certain properties of cancer associated mutations, optimal neoepitopes may be assessed and/or selected for inclusion in an mRNA vaccine. For example, at a given time, one or more of several properties may be assessed and weighted in order to select a set of neoepitopes for inclusion in a vaccine. A property of a neoepitope or set of neoepitopes may include, for instance, an assessment of gene or transcript-level expression in patient RNA-seq or other nucleic acid analysis, tissue-specific expression in available databases, known oncogenes/tumor suppressors, variant call confidence score, RNA-seq allele-specific expression, conservative vs. non-conservative AA substitution, position of point mutation (Centering Score for increased TCR engagement), position of point mutation (Anchoring Score for differential HLA binding), Selfness: <100% core epitope homology with patient WES data, HLA-A and -B IC50 for 8 mers-1 liners, HLA-DRB1 IC50 for 15 mers-20 mers, promiscuity Score (i.e. number of patient HLAs predicted to bind), HLA-C IC50 for 8 mers-1 liners, HLA-DRB3-5 IC50 for 15 mers-20 mers, HLA-DQB1/A1 IC50 for 15 mers-20 mers, HLA-DPB1/A1 IC50 for 15 mers-20 mers, Class I vs Class II proportion, Diversity of patient HLA-A, -B and DRB1 allotypes covered, proportion of point mutation vs complex epitopes (e.g. frameshifts), and/or pseudo-epitope HLA binding scores.

In some embodiments, the properties of cancer associated mutations used to identify optimal neoepitopes are properties related to the type of mutation, abundance of mutation in patient sample, immunogenicity, lack of self-reactivity, and nature of peptide composition.

The type of mutation should be determined and considered as a factor in determining whether a putative epitope should be included in a vaccine. The type of mutation may vary. In some instances it may be desirable to include multiple different types of mutations in a single vaccine. In other instances a single type of mutation may be more desirable. A value for particular mutation can be weighted and calculated. In some embodiments, a particular mutation is a single nucleotide polymorphism (SNP). In some embodiments, a particular mutation is a complex variant, for example, a peptide sequence resulting from intron retention, complex splicing events, or insertion/deletion mutations changing the reading frame of a sequence.

The abundance of the mutation in patient sample may also be scored and factored into the decision of whether a putative epitope should be included in a vaccine. Highly abundant mutations may promote a more robust immune response.

The consideration of the immunogenicity is an important component in the selection of optimal neoepitopes for inclusion in a vaccine. Immunogenicity may be assessed for instance, by analyzing the MHC binding capacity of a neoepitope, HLA promiscuity, mutation position, predicted T cell reactivity, actual T cell reactivity, structure leading to particular conformations and resultant solvent exposure, and representation of specific amino acids. Known algorithms such as the NetMHC prediction algorithm can be used to predict capacity of a peptide to bind to common HLA-A and -B alleles. Structural assessment of a MHC bound peptide may also be conducted by in silico 3-dimensional analysis and/or protein docking programs. Use of a predicted epitope structure when bound to a MHC molecule, such as acquired from a Rosetta algorithm, may be used to evaluate the degree of solvent exposure of an amino acid residues of an epitope when the epitope is bound to a MHC molecule. T cell reactivity may be assessed experimentally with epitopes and T cells in vitro. Alternatively T cell reactivity may be assessed using T cell response/sequence datasets.

An important component of a neoepitope included in a vaccine, is a lack of self-reactivity. The putative neoepitopes may be screened to confirm that the epitope is restricted to tumor tissue, for instance, arising as a result of genetic change within malignant cells. Ideally, the epitope should not be present in normal tissue of the patient and thus, self-similar epitopes are filtered out of the dataset. A personalized coding genome may be used as a reference for comparison of neoantigen candidates to determine lack of self-reactivity. In some embodiments, a personalized coding genome is generated from an individualized transcriptome and/or exome.

The nature of peptide composition may also be considered in the epitope design. For instance a score can be provided for each putative epitope on the value of conserved versus non-conserved amino acids found in the epitope.

In some embodiments, the analysis performed by the tools described herein may include comparing different sets of properties acquired at different times from a patient, i.e. prior to and following a therapeutic intervention, from different tissue samples, from different patients having similar tumors, etc. In some embodiments, an average of peak values from one set of properties may be compared with an average of peak values from another set of properties. For example, an average value for HLA binding may be compared between two different sets of distributions. The two sets of distributions may be determined for time durations separated by days, months, or years, for instance.

Moreover, the inventors have recognized and appreciated that such data on properties of cancer mutations may be collected and analyzed using the algorithms described herein. The data is useful for identifying neoepitopes and sets of neoepitopes for the development of personalized cancer vaccines.

In some embodiments, all annotated transcripts of a tumor variant peptide are included in a vaccine in accordance with the invention. In some embodiments, translations of RNA identified in RNAseq are included in a vaccine in accordance with the present invention.

It will be appreciated that a concatamer of 2 or more peptides, e.g., 2 or more neoantigens, may create unintended new epitopes (pseudoepitopes) at peptide boundaries. To prevent or eliminate such pseudoepitopes, class I alleles may be scanned for hits across peptide boundaries in a concatamer. In some embodiments, the peptide order within the concatamer is shuffled to reduce or eliminate pseudoepitope formation. In some embodiments, a linker is used between peptides, e.g., a single amino acid linker such as glycine, to reduce or eliminate pseudoepitope formation. In some embodiments, anchor amino acids can be replaced with other amino acids which will reduce or eliminate pseudoepitope formation. In some embodiments, peptides are trimmed at the peptide boundary within the concatamer to reduce or eliminate pseudoepitope formation.

In some embodiments the multiple peptide epitope antigens are arranged and ordered to minimize pseudoepitopes. In other embodiments the multiple peptide epitope antigens are a polypeptide that is free of pseudoepitopes. When the cancer antigen epitopes are arranged in a concatemeric structure in a head to tail formation a junction is formed between each of the cancer antigen epitopes. That includes several, i.e. 1-10, amino acids from an epitope on a N-terminus of the peptide and several, i.e. 1-10, amino acids on a C-terminus of an adjacent directly linked epitope. It is important that the junction not be an immunogenic peptide that may produce an immune response. In some embodiments the junction forms a peptide sequence that binds to an HLA protein of a subject for which the personalized cancer vaccine is designed with an IC50 greater than about 50 nM. In other embodiments the junction peptide sequence binds to an HLA protein of a subject with an IC50 greater than about 10 nM, 150 nM, 200 nM, 250 nM, 300 nM, 350 nM, 400 nM, 450 nm, or 500 nM.

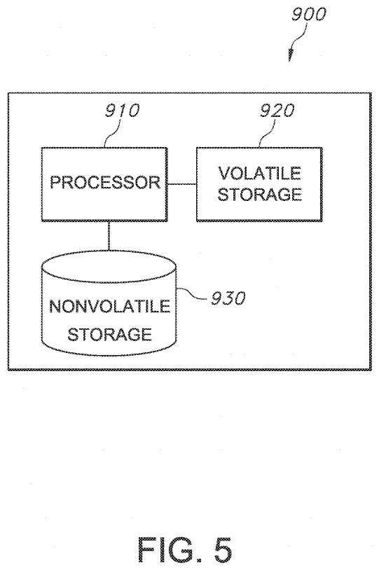

A neoepitope characterization system in accordance with the techniques described herein may take any suitable form, as embodiments are not limited in this respect. An illustrative implementation of a computer system 900 that may be used in connection with some embodiments is shown in FIG. 5. One or more computer systems such as computer system 900 may be used to implement any of the functionality described above. The computer system 900 may include one or more processors 910 and one or more computer-readable storage media (i.e., tangible, non-transitory computer-readable media), e.g., volatile storage 920 and one or more non-volatile storage media 930, which may be formed of any suitable data storage media. The processor 910 may control writing data to and reading data from the volatile storage 920 and the non-volatile storage device 930 in any suitable manner, as embodiments are not limited in this respect. To perform any of the functionality described herein, the processor 910 may execute one or more instructions stored in one or more computer-readable storage media (e.g., volatile storage 920 and/or non-volatile storage 930), which may serve as tangible, non-transitory computer-readable media storing instructions for execution by the processor 910.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation comprises at least one computer-readable storage medium (i.e., at least one tangible, non-transitory computer-readable medium), such as a computer memory (e.g., hard drive, flash memory, processor working memory, etc.), a floppy disk, an optical disk, a magnetic tape, or other tangible, non-transitory computer-readable medium, encoded with a computer program (i.e., a plurality of instructions), which, when executed on one or more processors, performs above-discussed functions. The computer-readable storage medium can be transportable such that the program stored thereon can be loaded onto any computer resource to implement techniques discussed herein. In addition, it should be appreciated that the reference to a computer program which, when executed, performs above-discussed functions, is not limited to an application program running on a host computer. Rather, the term “computer program” is used herein in a generic sense to reference any type of computer code (e.g., software or microcode) that can be employed to program one or more processors to implement above-techniques.

### GC-Rich Domains

### Definitions

GC-rich: As used herein, the term “GC-rich” refers to the nucleobase composition of a polynucleotide (e.g., mRNA), or any portion thereof (e.g., an RNA element), comprising guanine (G) and/or cytosine (C) nucleobases, or derivatives or analogs thereof, wherein the GC-content is greater than about 50%. The term “GC-rich” refers to all, or to a portion, of a polynucleotide, including, but not limited to, a gene, a non-coding region, a 5′ UTR, a 3′ UTR, an open reading frame, an RNA element, a sequence motif, or any discrete sequence, fragment, or segment thereof which comprises about 50% GC-content. In some embodiments of the disclosure, GC-rich polynucleotides, or any portions thereof, are exclusively comprised of guanine (G) and/or cytosine (C) nucleobases.

GC-content: As used herein, the term “GC-content” refers to the percentage of nucleobases in a polynucleotide (e.g., mRNA), or a portion thereof (e.g., an RNA element), that are either guanine (G) and cytosine (C) nucleobases, or derivatives or analogs thereof, (from a total number of possible nucleobases, including adenine (A) and thymine (T) or uracil (U), and derivatives or analogs thereof, in DNA and in RNA). The term “GC-content” refers to all, or to a portion, of a polynucleotide, including, but not limited to, a gene, a non-coding region, a 5′ or 3′ UTR, an open reading frame, an RNA element, a sequence motif, or any discrete sequence, fragment, or segment thereof.

Initiation Codon: As used herein, the term “initiation codon”, used interchangeably with the term “start codon”, refers to the first codon of an open reading frame that is translated by the ribosome and is comprised of a triplet of linked adenine-uracil-guanine nucleobases. The initiation codon is depicted by the first letter codes of adenine (A), uracil (U), and guanine (G) and is often written simply as “AUG”. Although natural mRNAs may use codons other than AUG as the initiation codon, which are referred to herein as “alternative initiation codons”, the initiation codons of polynucleotides described herein use the AUG codon. During the process of translation initiation, the sequence comprising the initiation codon is recognized via complementary base-pairing to the anticodon of an initiator tRNA (Met-tRNAᵢ^{Met}) bound by the ribosome. Open reading frames may contain more than one AUG initiation codon, which are referred to herein as “alternate initiation codons”.

The initiation codon plays a critical role in translation initiation. The initiation codon is the first codon of an open reading frame that is translated by the ribosome. Typically, the initiation codon comprises the nucleotide triplet AUG, however, in some instances translation initiation can occur at other codons comprised of distinct nucleotides. The initiation of translation in eukaryotes is a multistep biochemical process that involves numerous protein-protein, protein-RNA, and RNA-RNA interactions between messenger RNA molecules (mRNAs), the 40S ribosomal subunit, other components of the translation machinery (e.g., eukaryotic initiation factors; eIFs). The current model of mRNA translation initiation postulates that the pre-initiation complex (alternatively “43 S pre-initiation complex”; abbreviated as “PIC”) translocates from the site of recruitment on the mRNA (typically the 5′ cap) to the initiation codon by scanning nucleotides in a 5′ to 3′ direction until the first AUG codon that resides within a specific translation-promotive nucleotide context (the Kozak sequence) is encountered (Kozak (1989) J Cell Biol 108:229-241). Scanning by the PIC ends upon complementary base-pairing between nucleotides comprising the anticodon of the initiator Met-tRNAᵢ^{Met} transfer RNA and nucleotides comprising the initiation codon of the mRNA. Productive base-pairing between the AUG codon and the Met-tRNAᵢ^{Met} anticodon elicits a series of structural and biochemical events that culminate in the joining of the large 60S ribosomal subunit to the PIC to form an active ribosome that is competent for translation elongation.

Kozak Sequence: The term “Kozak sequence” (also referred to as “Kozak consensus sequence”) refers to a translation initiation enhancer element to enhance expression of a gene or open reading frame, and which in eukaryotes, is located in the 5′ UTR. The Kozak consensus sequence was originally defined as the sequence GCCRCC, where R=a purine, following an analysis of the effects of single mutations surrounding the initiation codon (AUG) on translation of the preproinsulin gene (Kozak (1986) Cell 44:283-292). Polynucleotides disclosed herein comprise a Kozak consensus sequence, or a derivative or modification thereof. (Examples of translational enhancer compositions and methods of use thereof, see U.S. Pat. No. 5,807,707 to Andrews et al., incorporated herein by reference in its entirety; U.S. Pat. No. 5,723,332 to Chernajovsky, incorporated herein by reference in its entirety; U.S. Pat. No. 5,891,665 to Wilson, incorporated herein by reference in its entirety.)

Leaky scanning: A phenomenon known as “leaky scanning” can occur whereby the PIC bypasses the initiation codon and instead continues scanning downstream until an alternate or alternative initiation codon is recognized. Depending on the frequency of occurrence, the bypass of the initiation codon by the PIC can result in a decrease in translation efficiency. Furthermore, translation from this downstream AUG codon can occur, which will result in the production of an undesired, aberrant translation product that may not be capable of eliciting the desired therapeutic response. In some cases, the aberrant translation product may in fact cause a deleterious response (Kracht et al, (2017) Nat Med 23(4):501-507).

Modified: As used herein “modified” or “modification” refers to a changed state or a change in composition or structure of a polynucleotide (e.g., mRNA). Polynucleotides may be modified in various ways including chemically, structurally, and/or functionally. For example, polynucleotides may be structurally modified by the incorporation of one or more RNA elements, wherein the RNA element comprises a sequence and/or an RNA secondary structure(s) that provides one or more functions (e.g., translational regulatory activity). Accordingly, polynucleotides of the disclosure may be comprised of one or more modifications (e.g., may include one or more chemical, structural, or functional modifications, including any combination thereof).

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

Nucleoside/Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Nucleic acid: As used herein, the term “nucleic acid” is used in its broadest sense and encompasses any compound and/or substance that includes a polymer of nucleotides, or derivatives or analogs thereof. These polymers are often referred to as “polynucleotides”. Accordingly, as used herein the terms “nucleic acid” and “polynucleotide” are equivalent and are used interchangeably. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, mRNAs, modified mRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Nucleic Acid Structure: As used herein, the term “nucleic acid structure” (used interchangeably with “polynucleotide structure”) refers to the arrangement or organization of atoms, chemical constituents, elements, motifs, and/or sequence of linked nucleotides, or derivatives or analogs thereof, that comprise a nucleic acid (e.g., an mRNA). The term also refers to the two-dimensional or three-dimensional state of a nucleic acid. Accordingly, the term “RNA structure” refers to the arrangement or organization of atoms, chemical constituents, elements, motifs, and/or sequence of linked nucleotides, or derivatives or analogs thereof, comprising an RNA molecule (e.g., an mRNA) and/or refers to a two-dimensional and/or three dimensional state of an RNA molecule. Nucleic acid structure can be further demarcated into four organizational categories referred to herein as “molecular structure”, “primary structure”, “secondary structure”, and “tertiary structure” based on increasing organizational complexity.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Pre-Initiation Complex (PIC): As used herein, the term “pre-initiation complex” (alternatively “43 S pre-initiation complex”; abbreviated as “PIC”) refers to a ribonucleoprotein complex comprising a 40S ribosomal subunit, eukaryotic initiation factors (eIF1, eIF1A, eIF3, eIF5), and the eIF2-GTP-Met-tRNAᵢ^{Met} ternary complex, that is intrinsically capable of attachment to the 5′ cap of an mRNA molecule and, after attachment, of performing ribosome scanning of the 5′ UTR.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2): 194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al, (2007) Nat Rev Mol Cell Biol 8(2): 113-126), translational repression element (see e.g., Blumer et al., (2002) Mech Dev 110(1-2):97-112), protein-binding RNA elements (e.g., iron-responsive element, see Selezneva et a., (2013) J Mol Biol 425(18):3301-3310), cytoplasmic polyadenylation elements (Villalba et al, (2011) Curr Opin Genet Dev 21(4):452-457), and catalytic RNA elements (e.g., ribozymes, see Scott et al, (2009) Biochim Biophys Acta 1789(9-10):634-641).

Residence time: As used herein, the term “residence time” refers to the time of occupancy of a pre-initiation complex (PIC) or a ribosome at a discrete position or location along an mRNA molecule.

Translational Regulatory Activity: As used herein, the term “translational regulatory activity” (used interchangeably with “translational regulatory function”) refers to a biological function, mechanism, or process that modulates (e.g., regulates, influences, controls, varies) the activity of the translational apparatus, including the activity of the PIC and/or ribosome. In some aspects, the desired translation regulatory activity promotes and/or enhances the translational fidelity of mRNA translation. In some aspects, the desired translational regulatory activity reduces and/or inhibits leaky scanning.

Translation of a polynucleotide comprising an open reading frame encoding a polypeptide can be controlled and regulated by a variety of mechanisms that are provided by various cis-acting nucleic acid structures. For example, naturally-occurring, cis-acting RNA elements that form hairpins or other higher-order (e.g., pseudoknot) intramolecular mRNA secondary structures can provide a translational regulatory activity to a polynucleotide, wherein the RNA element influences or modulates the initiation of polynucleotide translation, particularly when the RNA element is positioned in the 5′ UTR close to the 5′-cap structure (Pelletier and Sonenberg (1985) Cell 40(3):515-526; Kozak (1986) Proc Natl Acad Sci 83:2850-2854). Cis-acting RNA elements can also affect translation elongation, being involved in numerous frameshifting events (Namy et al, (2004) Mol Cell 13(2):157-168). Internal ribosome entry sequences (IRES) represent another type of cis-acting RNA element that are typically located in 5′ UTRs, but have also been reported to be found within the coding region of naturally-occurring mRNAs (Holcik et al (2000) Trends Genet 16(10):469-473). In cellular mRNAs, IRES often coexist with the 5′-cap structure and provide mRNAs with the functional capacity to be translated under conditions in which cap-dependent translation is compromised (Gebauer et al., (2012) Cold Spring Harb Perspect Biol 4(7):a012245). Another type of naturally-occurring cis-acting RNA element comprises upstream open reading frames (uORFs). Naturally-occurring uORFs occur singularly or multiply within the 5′ UTRs of numerous mRNAs and influence the translation of the downstream maj or ORF, usually negatively (with the notable exception of GCN4 mRNA in yeast and ATF4 mRNA in mammals, where uORFs serve to promote the translation of the downstream maj or ORF under conditions of increased eIF2 phosphorylation (Hinnebusch (2005) Annu Rev Microbiol 59:407-450)). Additional exemplary translational regulatory activities provided by components, structures, elements, motifs, and/or specific sequences comprising polynucleotides (e.g., mRNA) include, but are not limited to, mRNA stabilization or destabilization (Baker & Parker (2004) Curr Opin Cell Biol 16(3):293-299), translational activation (Villalba et al., (2011) Curr Opin Genet Dev 21(4):452-457), and translational repression (Blumer et al, (2002) Mech Dev 110(1-2):97-112). Studies have shown that naturally-occurring, cis-acting RNA elements can confer their respective functions when used to modify, by incorporation into, heterologous polynucleotides (Goldberg-Cohen et at, (2002) J Biol Chem 277(16):13635-13640).

### Modified Polynucleotides Comprising Functional RNA Elements

The present disclosure provides synthetic polynucleotides comprising a modification (e.g., an RNA element), wherein the modification provides a desired translational regulatory activity. In some embodiments, the disclosure provides a polynucleotide comprising a 5′ untranslated region (UTR), an initiation codon, a full open reading frame encoding a polypeptide, a 3′ UTR, and at least one modification, wherein the at least one modification provides a desired translational regulatory activity, for example, a modification that promotes and/or enhances the translational fidelity of mRNA translation. In some embodiments, the desired translational regulatory activity is a cis-acting regulatory activity. In some embodiments, the desired translational regulatory activity is an increase in the residence time of the 43 S pre-initiation complex (PIC) or ribosome at, or proximal to, the initiation codon. In some embodiments, the desired translational regulatory activity is an increase in the initiation of polypeptide synthesis at or from the initiation codon. In some embodiments, the desired translational regulatory activity is an increase in the amount of polypeptide translated from the full open reading frame. In some embodiments, the desired translational regulatory activity is an increase in the fidelity of initiation codon decoding by the PIC or ribosome. In some embodiments, the desired translational regulatory activity is inhibition or reduction of leaky scanning by the PIC or ribosome. In some embodiments, the desired translational regulatory activity is a decrease in the rate of decoding the initiation codon by the PIC or ribosome. In some embodiments, the desired translational regulatory activity is inhibition or reduction in the initiation of polypeptide synthesis at any codon within the mRNA other than the initiation codon. In some embodiments, the desired translational regulatory activity is inhibition or reduction of the amount of polypeptide translated from any open reading frame within the mRNA other than the full open reading frame. In some embodiments, the desired translational regulatory activity is inhibition or reduction in the production of aberrant translation products. In some embodiments, the desired translational regulatory activity is a combination of one or more of the foregoing translational regulatory activities.

Accordingly, the present disclosure provides a polynucleotide, e.g., an mRNA, comprising an RNA element that comprises a sequence and/or an RNA secondary structure(s) that provides a desired translational regulatory activity as described herein. In some aspects, the mRNA comprises an RNA element that comprises a sequence and/or an RNA secondary structure(s) that promotes and/or enhances the translational fidelity of mRNA translation. In some aspects, the mRNA comprises an RNA element that comprises a sequence and/or an RNA secondary structure(s) that provides a desired translational regulatory activity, such as inhibiting and/or reducing leaky scanning. In some aspects, the disclosure provides an mRNA that comprises an RNA element that comprises a sequence and/or an RNA secondary structure(s) that inhibits and/or reduces leaky scanning thereby promoting the translational fidelity of the mRNA.

In some embodiments, the RNA element comprises natural and/or modified nucleotides. In some embodiments, the RNA element comprises of a sequence of linked nucleotides, or derivatives or analogs thereof, that provides a desired translational regulatory activity as described herein. In some embodiments, the RNA element comprises a sequence of linked nucleotides, or derivatives or analogs thereof, that forms or folds into a stable RNA secondary structure, wherein the RNA secondary structure provides a desired translational regulatory activity as described herein. RNA elements can be identified and/or characterized based on the primary sequence of the element (e.g., GC-rich element), by RNA secondary structure formed by the element (e.g. stem-loop), by the location of the element within the RNA molecule (e.g., located within the 5′ UTR of an mRNA), by the biological function and/or activity of the element (e.g., “translational enhancer element”), and any combination thereof.

In some aspects, the disclosure provides an mRNA having one or more structural modifications that inhibits leaky scanning and/or promotes the translational fidelity of mRNA translation, wherein at least one of the structural modifications is a GC-rich RNA element. In some aspects, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In any of the foregoing or related aspects, the disclosure provides a GC-rich RNA element which comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is 70-80% cytosine, 60-70% cytosine, 50%-60% cytosine, 40-50% cytosine, 30-40% cytosine bases. In any of the foregoing or related aspects, the disclosure provides a GC-rich RNA element which comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is about 80% cytosine, about 70% cytosine, about 60% cytosine, about 50% cytosine, about 40% cytosine, or about 30% cytosine.

In any of the foregoing or related aspects, the disclosure provides a GC-rich RNA element which comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is 70-80% cytosine, 60-70% cytosine, 50%-60% cytosine, 40-50% cytosine, or 30-40% cytosine. In any of the foregoing or related aspects, the disclosure provides a GC-rich RNA element which comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is about 80% cytosine, about 70% cytosine, about 60% cytosine, about 50% cytosine, about 40% cytosine, or about 30% cytosine.

In some embodiments, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of 3, 4, 5, 6, 7, 8, 9, 10, 11, 12, 13, 14, 15, 16, 17, 18, 19, or 20 nucleotides, or derivatives or analogs thereof, linked in any order, wherein the sequence composition is >50% cytosine. In some embodiments, the sequence composition is >55% cytosine, >60% cytosine, >65% cytosine, >70% cytosine, >75% cytosine, >80% cytosine, >85% cytosine, or >90% cytosine.

In other aspects, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of about 3-30, 5-25, 10-20, 15-20 or about 20, about 15, about 12, about 10, about 6 or about 3 nucleotides, or derivatives or analogues thereof, wherein the sequence comprises a repeating GC-motif, wherein the repeating GC-motif is [CCG]n, wherein n=1 to 10, n=2 to 8, n=3 to 6, or n=4 to 5. In some embodiments, the sequence comprises a repeating GC-motif [CCG]n, wherein n=1, 2, 3, 4 or 5. In some embodiments, the sequence comprises a repeating GC-motif [CCG]n, wherein n=1, 2, or 3. In some embodiments, the sequence comprises a repeating GC-motif [CCG]n, wherein n=1. In some embodiments, the sequence comprises a repeating GC-motif [CCG]n, wherein n=2. In some embodiments, the sequence comprises a repeating GC-motif [CCG]n, wherein n=3. In some embodiments, the sequence comprises a repeating GC-motif [CCG]n, wherein n=4 (SEQ ID NO: 308). In some embodiments, the sequence comprises a repeating GC-motif [CCG]n, wherein n=5 (SEQ ID NO: 309).

In another aspect, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element comprises any one of the sequences set forth in TABLE 2. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located about 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In other aspects, the disclosure provides a modified mRNA comprising at least one modification, wherein at least one modification is a GC-rich RNA element comprising the sequence V1 [CCCCGGCGCC] (SEQ ID NO: 310) as set forth in TABLE 2, or derivatives or analogs thereof, preceding a Kozak consensus sequence in the 5′ UTR of the mRNA. In some embodiments, the GC-rich element comprises the sequence V1 as set forth in TABLE 2 located immediately adjacent to and upstream of the Kozak consensus sequence in the 5′ UTR of the mRNA. In some embodiments, the GC-rich element comprises the sequence V1 as set forth in TABLE 2 located 1, 2, 3, 4, 5, 6, 7, 8, 9 or 10 bases upstream of the Kozak consensus sequence in the 5′ UTR of the mRNA. In other embodiments, the GC-rich element comprises the sequence V1 as set forth in TABLE 2 located 1-3, 3-5, 5-7, 7-9, 9-12, or 12-15 bases upstream of the Kozak consensus sequence in the 5′ UTR of the mRNA.

In other aspects, the disclosure provides a modified mRNA comprising at least one modification, wherein at least one modification is a GC-rich RNA element comprising the sequence V2 [CCCCGGC] as set forth in TABLE 2, or derivatives or analogs thereof, preceding a Kozak consensus sequence in the 5′ UTR of the mRNA. In some embodiments, the GC-rich element comprises the sequence V2 as set forth in TABLE 2 located immediately adjacent to and upstream of the Kozak consensus sequence in the 5′ UTR of the mRNA. In some embodiments, the GC-rich element comprises the sequence V2 as set forth in TABLE 2 located 1, 2, 3, 4, 5, 6, 7, 8, 9 or 10 bases upstream of the Kozak consensus sequence in the 5′ UTR of the mRNA. In other embodiments, the GC-rich element comprises the sequence V2 as set forth in TABLE 2 located 1-3, 3-5, 5-7, 7-9, 9-12, or 12-15 bases upstream of the Kozak consensus sequence in the 5′ UTR of the mRNA.

In other aspects, the disclosure provides a modified mRNA comprising at least one modification, wherein at least one modification is a GC-rich RNA element comprising the sequence EK [GCCGCC] as set forth in TABLE 2, or derivatives or analogs thereof, preceding a Kozak consensus sequence in the 5′ UTR of the mRNA. In some embodiments, the GC-rich element comprises the sequence EK as set forth in TABLE 2 located immediately adjacent to and upstream of the Kozak consensus sequence in the 5′ UTR of the mRNA. In some embodiments, the GC-rich element comprises the sequence EK as set forth in TABLE 2 located 1, 2, 3, 4, 5, 6, 7, 8, 9 or 10 bases upstream of the Kozak consensus sequence in the 5′ UTR of the mRNA. In other embodiments, the GC-rich element comprises the sequence EK as set forth in TABLE 2 located 1-3, 3-5, 5-7, 7-9, 9-12, or 12-15 bases upstream of the Kozak consensus sequence in the 5′ UTR of the mRNA.

In yet other aspects, the disclosure provides a modified mRNA comprising at least one modification, wherein at least one modification is a GC-rich RNA element comprising the sequence VI [CCCCGGCGCC] (SEQ ID NO: 310) as set forth in TABLE 2, or derivatives or analogs thereof, preceding a Kozak consensus sequence in the 5′ UTR of the mRNA, wherein the 5′ UTR comprises the following sequence shown in TABLE 2:

(SEQ ID NO: 311)
GGGAAATAAGAGAGAAAAGAAGAGTAAGAAGAAATATAAGA.

In some embodiments, the GC-rich element comprises the sequence V1 as set forth in TABLE 2 located immediately adjacent to and upstream of the Kozak consensus sequence in the 5′ UTR sequence shown in TABLE 2. In some embodiments, the GC-rich element comprises the sequence V1 as set forth in TABLE 2 located 1, 2, 3, 4, 5, 6, 7, 8, 9 or 10 bases upstream of the Kozak consensus sequence in the 5′ UTR of the mRNA, wherein the 5′ UTR comprises the following sequence shown in TABLE 2:

(SEQ ID NO: 312)
GGGAAATAAGAGAGAAAAGAAGAGTAAGAAGAAATATAAGA.

In other embodiments, the GC-rich element comprises the sequence V1 as set forth in Table 1 located 1-3, 3-5, 5-7, 7-9, 9-12, or 12-15 bases upstream of the Kozak consensus sequence in the 5′ UTR of the mRNA, wherein the 5′ UTR comprises the following sequence shown in TABLE 2:

(SEQ ID NO: 312)
GGGAAATAAGAGAGAAAAGAAGAGTAAGAAGAAATATAAGA.

In some embodiments, the 5′ UTR comprises the following sequence set forth in TABLE 2:

(SEQ ID NO: 313)
GGGAAATAAGAGAGAAAAGAAGAGTAAGAAGAAATATAAGACCCCGGCGC
CGCCACC

TABLE 2

| SEQ ID | | |
|---|---|---|
| NO: | 5′ UTRs | 5′UTR Sequence |
| 314 | Standard | GGGAAATAAGAGAGAAAAGAAGAGTAAG |
| | | AAGAAATATAAGAGCCACC |
| 313 | V1-UTR | GGGAAATAAGAGAGAAAAGAAGAGTAAG |
| | | AAGAAATATAAGACCCCGGCGCCGCCACC |
| 315 | V2-UTR | GGGAAATAAGAGAGAAAAGAAGAGTAAG |
| | | AAGAAATATAAGACCCCGGCGCCACC |
| | GC-Rich RNA Elements | Sequence |
| | K0 (Traditional Kozak | [GCCA/GCC] |
| | consensus) | |
| | EK | [GCCGCC] |
| 310 | V1 | [CCCCGGCGCC] |
| | V2 | [CCCCGGC] |
| | (CCG)ₙ, where n = 1-10 | [CCG]ₙ |
| | (GCC)ₙ, where n = 1-10 | [GCC]ₙ |

In another aspect, the disclosure provides a modified mRNA comprising at least one modification, wherein at least one modification is a GC-rich RNA element comprising a stable RNA secondary structure comprising a sequence of nucleotides, or derivatives or analogs thereof, linked in an order which forms a hairpin or a stem-loop. In one embodiment, the stable RNA secondary structure is upstream of the Kozak consensus sequence. In another embodiment, the stable RNA secondary structure is located about 30, about 25, about 20, about 15, about 10, or about 5 nucleotides upstream of the Kozak consensus sequence. In another embodiment, the stable RNA secondary structure is located about 20, about 15, about 10 or about 5 nucleotides upstream of the Kozak consensus sequence. In another embodiment, the stable RNA secondary structure is located about 5, about 4, about 3, about 2, about 1 nucleotides upstream of the Kozak consensus sequence. In another embodiment, the stable RNA secondary structure is located about 15-30, about 15-20, about 15-25, about 10-15, or about 5-10 nucleotides upstream of the Kozak consensus sequence. In another embodiment, the stable RNA secondary structure is located 12-15 nucleotides upstream of the Kozak consensus sequence. In another embodiment, the stable RNA secondary structure has a deltaG of about −30 kcal/mol, about −20 to −30 kcal/mol, about −20 kcal/mol, about −10 to −20 kcal/mol, about −10 kcal/mol, about −5 to −10 kcal/mol.

In another embodiment, the modification is operably linked to an open reading frame encoding a polypeptide and wherein the modification and the open reading frame are heterologous.

In another embodiment, the sequence of the GC-rich RNA element is comprised exclusively of guanine (G) and cytosine (C) nucleobases.

RNA elements that provide a desired translational regulatory activity as described herein can be identified and characterized using known techniques, such as ribosome profiling. Ribosome profiling is a technique that allows the determination of the positions of PICs and/or ribosomes bound to mRNAs (see e.g., Ingolia et al., (2009) Science 324(5924):218-23, incorporated herein by reference). The technique is based on protecting a region or segment of mRNA, by the PIC and/or ribosome, from nuclease digestion. Protection results in the generation of a 30-bp fragment of RNA termed a ‘footprint’. The sequence and frequency of RNA footprints can be analyzed by methods known in the art (e.g., RNA-seq). The footprint is roughly centered on the A-site of the ribosome. If the PIC or ribosome dwells at a particular position or location along an mRNA, footprints generated at these position would be relatively common. Studies have shown that more footprints are generated at positions where the PIC and/or ribosome exhibits decreased processivity and fewer footprints where the PIC and/or ribosome exhibits increased processivity (Gardin et at, (2014) eLife 3:e03735). In some embodiments, residence time or the time of occupancy of a the PIC or ribosome at a discrete position or location along an polynucleotide comprising any one or more of the RNA elements described herein is determined by ribosome profiling.

### Methods of Treatment

Provided herein are compositions (e.g., pharmaceutical compositions), methods, kits and reagents for prevention and/or treatment of cancer in humans and other mammals. Cancer RNA vaccines can be used as therapeutic or prophylactic agents. They may be used in medicine to prevent and/or treat cancer. In exemplary aspects, the cancer RNA vaccines of the present disclosure are used to provide prophylactic protection from cancer. Prophylactic protection from cancer can be achieved following administration of a cancer RNA vaccine of the present disclosure. Vaccines can be administered once, twice, three times, four times or more but it is likely sufficient to administer the vaccine once (optionally followed by a single booster). It is more desirable, to administer the vaccine to an individual having cancer to achieve a therapeutic response. Dosing may need to be adjusted accordingly.

Once an mRNA vaccine is synthesized, it is administered to the patient. In some embodiments the vaccine is administered on a schedule for up to two months, up to three months, up to four month, up to five months, up to six months, up to seven months, up to eight months, up to nine months, up to ten months, up to eleven months, up to 1 year, up to 1 and ½ years, up to two years, up to three years, or up to four years. The schedule may be the same or varied. In some embodiments the schedule is weekly for the first 3 weeks and then monthly thereafter.

The vaccine may be administered by any route. In some embodiments the vaccine is administered by an IM or IV route.

At any point in the treatment the patient may be examined to determine whether the mutations in the vaccine are still appropriate. Based on that analysis the vaccine may be adjusted or reconfigured to include one or more different mutations or to remove one or more mutations.

### Therapeutic and Prophylactic Compositions

Provided herein are compositions (e.g., pharmaceutical compositions), methods, kits and reagents for prevention, treatment or diagnosis of cancer in humans and other mammals, For example, cancer RNA vaccines can be used as therapeutic or prophylactic agents. They may be used in medicine to prevent and/or treat cancer. In some embodiments, the cancer vaccines of the invention can be envisioned for use in the priming of immune effector cells, for example, to activate peripheral blood mononuclear cells (PBMCs) ex vivo, which are then infused (re-infused) into a subject.

In exemplary embodiments, a cancer vaccine containing RNA polynucleotides as described herein can be administered to a subject (e.g., a mammalian subject, such as a human subject), and the RNA polynucleotides are translated in vivo to produce an antigenic polypeptide.

The cancer RNA vaccines may be induced for translation of a polypeptide (e.g., antigen or immunogen) in a cell, tissue or organism. In exemplary embodiments, such translation occurs in vivo, although there can be envisioned embodiments where such translation occurs ex vivo, in culture or in vitro. In exemplary embodiments, the cell, tissue or organism is contacted with an effective amount of a composition containing a cancer RNA vaccine that contains a polynucleotide that has at least one a translatable region encoding an antigenic polypeptide.

An “effective amount” of a cancer RNA vaccine is provided based, at least in part, on the target tissue, target cell type, means of administration, physical characteristics of the polynucleotide (e.g., size, and extent of modified nucleosides) and other components of the cancer RNA vaccine, and other determinants. In general, an effective amount of the cancer RNA vaccine composition provides an induced or boosted immune response as a function of antigen production in the cell, preferably more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation from the polynucleotide, decreased nucleic acid degradation (as demonstrated, for example, by increased duration of protein translation from a modified polynucleotide), or altered antigen specific immune response of the host cell.

In some embodiments, RNA vaccines (including polynucleotides their encoded polypeptides) in accordance with the present disclosure may be used for treatment of cancer.

Cancer RNA vaccines may be administered prophylactically or therapeutically as part of an active immunization scheme to healthy individuals or early in cancer or during active cancer after onset of symptoms. In some embodiments, the amount of RNA vaccines of the present disclosure provided to a cell, a tissue or a subject may be an amount effective for immune prophylaxis.

Cancer RNA vaccines may be administered with other prophylactic or therapeutic compounds. As a non-limiting example, a prophylactic or therapeutic compound may be an immune potentiator, adjuvant, or booster. As used herein, when referring to a composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months or 1 year.

In one embodiment, the polynucleotides may be administered intramuscularly or intradermally similarly to the administration of vaccines known in the art.

The mRNA cancer vaccines may be utilized in various settings depending on the severity of the cancer or the degree or level of unmet medical need. As a non-limiting example, the mRNA cancer vaccines may be utilized to treat any stage of cancer. The mRNA cancer vaccines have superior properties in that they produce much larger antibody titers, T cell responses and produce responses early than commercially available anti-cancer vaccines. While not wishing to be bound by theory, the inventors hypothesize that the mRNA cancer vaccines, as mRNAs, are better designed to produce the appropriate protein conformation on translation as the mRNA cancer vaccines co-opt natural cellular machinery. Unlike traditional vaccines which are manufactured ex vivo and may trigger unwanted cellular responses, the mRNA cancer vaccines are presented to the cellular system in a more native fashion.

A non-limiting list of cancers that the mRNA cancer vaccines may treat is presented below. Peptide epitopes or antigens may be derived from any antigen of these cancers or tumors. Such epitopes are referred to as cancer or tumor antigens. Cancer cells may differentially express cell surface molecules during different phases of tumor progression. For example, a cancer cell may express a cell surface antigen in a benign state, yet down-regulate that particular cell surface antigen upon metastasis. As such, it is envisioned that the tumor or cancer antigen may encompass antigens produced during any stage of cancer progression. The methods of the invention may be adjusted to accommodate for these changes. For instance, several different mRNA vaccines may be generated for a particular patient. For instance a first vaccine may be used at the start of the treatment. At a later time point, a new mRNA vaccine may be generated and administered to the patient to account for different antigens being expressed.

In some embodiments, the tumor antigen is one of the following antigens: CD2, CD19, CD20, CD22, CD27, CD33, CD37, CD38, CD40, CD44, CD47, CD52, CD56, CD70, 30 CD79, CD137, 4-IBB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gplOO, gpA33, GPNMB, ICOS, IGF1R, Integrin av, Integrin av3, LAG-3, Lewis Y, Mesothelin, c-MET, MN Carbonic anhydrase IX, MUC1, MUC16, Nectin-4, NKGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2,VEGFR-1, VEGFR-2, VEGFR-3, and variants thereof.

Cancers or tumors include but are not limited to neoplasms, malignant tumors, metastases, or any disease or disorder characterized by uncontrolled cell growth such that it would be considered cancerous. The cancer may be a primary or metastatic cancer. Specific cancers that can be treated according to the present invention include, but are not limited to, those listed below (for a review of such disorders, see Fishman et al., 1985, Medicine, 2d Ed., J.B. Lippincott Co., Philadelphia). Cancers include, but are not limited to, biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma, teratomas, choriocarcinomas; stromal tumors and germ cell tumors; thyroid cancer including thyroid adenocarcinoma and medullar carcinoma; and renal cancer including adenocarcinoma and Wilms' tumor. Commonly encountered cancers include breast, prostate, lung, ovarian, colorectal, and brain cancer.

In some embodiments, the cancer is selected from the group consisting of non-small cell lung cancer (NSCLC), small cell lung cancer, melanoma, bladder urothelial carcinoma, HPV-negative head and neck squamous cell carcinoma (HNSCC), and a solid malignancy that is microsatellite high (MSI H)/mismatch repair (MMR) deficient. In some embodiments, the NSCLC lacks an EGFR sensitizing mutation and/or an ALK translocation. In some embodiments, the solid malignancy that is microsatellite high (MSI H)/mismatch repair (MMR) deficient is selected from the group consisting of colorectal cancer, stomach adenocarcinoma, esophageal adenocarcinoma, and endometrial cancer. In some embodiments, the cancer is selected from cancer of the pancreas, peritoneum, large intestine, small intestine, biliary tract, lung, endometrium, ovary, genital tract, gastrointestinal tract, cervix, stomach, urinary tract, colon, rectum, and hematopoietic and lymphoid tissues. In some embodiments, the cancer is colorectal cancer.

Provided herein are pharmaceutical compositions including cancer RNA vaccines and RNA vaccine compositions and/or complexes optionally in combination with one or more pharmaceutically acceptable excipients.

Cancer RNA vaccines may be formulated or administered alone or in conjunction with one or more other components. For instance, cancer RNA vaccines (vaccine compositions) may comprise other components including, but not limited to, immune potentiators (e.g., adjuvants). In some embodiments, cancer RNA vaccines do not include an immune potentiator or adjuvant (i.e., they are immune potentiator or adjuvant free).

In other embodiments the mRNA cancer vaccines described herein may be combined with any other therapy useful for treating the patient. For instance a patient may be treated with the mRNA cancer vaccine and an anti-cancer agent. Thus, in one embodiment, the methods of the invention can be used in conjunction with one or more cancer therapeutics, for example, in conjunction with an anti-cancer agent, a traditional cancer vaccine, chemotherapy, radiotherapy, etc. (e.g., simultaneously, or as part of an overall treatment procedure). Parameters of cancer treatment that may vary include, but are not limited to, dosages, timing of administration or duration or therapy; and the cancer treatment can vary in dosage, timing, or duration. Another treatment for cancer is surgery, which can be utilized either alone or in combination with any of the previous treatment methods. Any agent or therapy (e.g., traditional cancer vaccines, chemotherapies, radiation therapies, surgery, hormonal therapies, and/or biological therapies/immunotherapies) which is known to be useful, or which has been used or is currently being used for the prevention or treatment of cancer can be used in combination with a composition of the invention in accordance with the invention described herein. One of ordinary skill in the medical arts can determine an appropriate treatment for a subject.

Examples of such agents (i.e., anti-cancer agents) include, but are not limited to, DNA-interactive agents including, but not limited to, the alkylating agents (e.g., nitrogen mustards, e.g. Chlorambucil, Cyclophosphamide, Isofamide, Mechlorethamine, Melphalan, Uracil mustard; Aziridine such as Thiotepa; methanesulphonate esters such as Busulfan; nitroso ureas, such as Carmustine, Lomustine, Streptozocin; platinum complexes, such as Cisplatin, Carboplatin; bioreductive alkylator, such as Mitomycin, and Procarbazine, Dacarbazine and Altretamine); the DNA strand-breakage agents, e.g., Bleomycin; the intercalating topoisomerase II inhibitors, e.g., Intercalators, such as Amsacrine, Dactinomycin, Daunorubicin, Doxorubicin, Idarubicin, Mitoxantrone, and nonintercalators, such as Etoposide and Teniposide; the nonintercalating topoisomerase II inhibitors, e.g., Etoposide and Teniposde; and the DNA minor groove binder, e.g., Plicamydin; the antimetabolites including, but not limited to, folate antagonists such as Methotrexate and trimetrexate; pyrimidine antagonists, such as Fluorouracil, Fluorodeoxyuridine, CB3717, Azacitidine and Floxuridine; purine antagonists such as Mercaptopurine, 6-Thioguanine, Pentostatin; sugar modified analogs such as Cytarabine and Fludarabine; and ribonucleotide reductase inhibitors such as hydroxyurea; tubulin Interactive agents including, but not limited to, colcbicine, Vincristine and Vinblastine, both alkaloids and Paclitaxel and cytoxan; hormonal agents including, but not limited to, estrogens, conjugated estrogens and Ethinyl Estradiol and Diethylstilbesterol, Chlortrianisen and Idenestrol; progestins such as Hydroxyprogesterone caproate, Medroxyprogesterone, and Megestrol; and androgens such as testosterone, testosterone propionate; fluoxymesterone, methyltestosterone; adrenal corticosteroid, e.g., Prednisone, Dexamethasone, Methylprednisolone, and Prednisolone; leutinizing hormone releasing hormone agents or gonadotropin-releasing hormone antagonists, e.g., leuprolide acetate and goserelin acetate; antihormonal antigens including, but not limited to, antiestrogenic agents such as Tamoxifen, antiandrogen agents such as Flutamide; and antiadrenal agents such as Mitotane and Aminoglutethimide; cytokines including, but not limited to, IL-1.alpha., IL-1 0, IL-2, IL-3, IL-4, IL-5, IL-6, IL-7, IL-8, IL-9, IL-10, IL-11, IL-12, IL-13, IL-18, TGF-β, GM-CSF, M-CSF, G-CSF, TNF-α, TNF-β, LAF, TCGF, BCGF, TRF, BAF, BDG, MP, LIF, OSM, TMF, PDGF, IFN-α, IFN-β, IFN-γ, and Uteroglobins (U.S. Pat. No. 5,696,092); anti-angiogenics including, but not limited to, agents that inhibit VEGF (e.g., other neutralizing antibodies), soluble receptor constructs, tyrosine kinase inhibitors, antisense strategies, RNA aptamers and ribozymes against VEGF or VEGF receptors, Immunotoxins and coaguligands, tumor vaccines, and antibodies.

Specific examples of anti-cancer agents which can be used in accordance with the methods of the invention include, but not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interieukin II, or rIL2), interferon alpha-2a; interferon alpha-2b; interferon alpha-nl; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; angiogenesis inhibitors; anti-dorsalizing morphogenetic protein-1; ara-CDP-DL-PTBA; BCR/ABL antagonists; CaRest M3; CARN 700; casein kinase inhibitors (ICOS); clotrimazole; collismycin A; collismycin B; combretastatin A4; crambescidin 816; cryptophycin 8; curacin A; dehydrodidemnin B; didemnin B; dihydro-5-azacytidine; dihydrotaxol, duocarmycin SA; kahalalide F; lamellarin-N triacetate; leuprolide+estrogen+progesterone; lissoclinamide 7; monophosphoryl lipid A+myobacterium cell wall sk; N-acetyldinaline; N-substituted benzamides; 06-benzylguanine; placetin A; placetin B; platinum complex; platinum compounds; platinum-triamine complex; rhenium Re 186 etidronate; RII retinamide; rubiginone B1; SarCNU; sarcophytol A; sargramostim; senescence derived inhibitor 1; spicamycin D; tallimustine; 5-fluorouracil; thrombopoietin; thymotrinan; thyroid stimulating hormone; variolin B; thalidomide; velaresol; veramine; verdins; verteporfin; vinorelbine; vinxaltine; vitaxin; zanoterone; zeniplatin; and zilascorb.

The invention also encompasses administration of a composition comprising a mRNA cancer vaccine in combination with radiation therapy comprising the use of x-rays, gamma rays and other sources of radiation to destroy the cancer cells. In preferred embodiments, the radiation treatment is administered as external beam radiation or teletherapy wherein the radiation is directed from a remote source. In other preferred embodiments, the radiation treatment is administered as internal therapy or brachytherapy wherein a radioactive source is placed inside the body close to cancer cells or a tumor mass.

In specific embodiments, an appropriate anti-cancer regimen is selected depending on the type of cancer. For instance, a patient with ovarian cancer may be administered a prophylactically or therapeutically effective amount of a composition comprising a mRNA cancer vaccine in combination with a prophylactically or therapeutically effective amount of one or more other agents useful for ovarian cancer therapy, including but not limited to, intraperitoneal radiation therapy, such as P32 therapy, total abdominal and pelvic radiation therapy, cisplatin, the combination of paclitaxel (Taxol) or docetaxel (Taxotere) and cisplatin or carboplatin, the combination of cyclophosphamide and cisplatin, the combination of cyclophosphamide and carboplatin, the combination of 5-FU and leucovorin, etoposide, liposomal doxorubicin, gemcitabine or topotecan. Cancer therapies and their dosages, routes of administration and recommended usage are known in the art and have been described in such literature as the Physician's Desk Reference (56th ed., 2002).

In some preferred embodiments of the invention the mRNA cancer vaccines are administered with a T cell activator such as be an immune checkpoint modulator. Immune checkpoint modulators include both stimulatory checkpoint molecules and inhibitory checkpoint molecules i.e., an anti-CTLA4 and anti-PD1 antibody.

Stimulatory checkpoint inhibitors function by promoting the checkpoint process. Several stimulatory checkpoint molecules are members of the tumor necrosis factor (TNF) receptor superfamily—CD27, CD40, OX40, GITR and CD137, while others belong to the B7-CD28 superfamily—CD28 and ICOS. OX40 (CD134), is involved in the expansion of effector and memory T cells. Anti-OX40 monoclonal antibodies have been shown to be effective in treating advanced cancer. MEDI0562 is a humanized OX40 agonist. GITR, Glucocorticoid-Induced TNFR family Related gene, is involved in T cell expansion Several antibodies to GITR have been shown to promote an anti-tumor responses. ICOS, Inducible T-cell costimulator, is important in T cell effector function. CD27 supports antigen-specific expansion of naïve T cells and is involved in the generation of T and B cell memory. Several agonistic anti-CD27 antibodies are in development. CD122 is the Interleukin-2 receptor beta sub-unit. NKTR-214 is a CD122-biased immune-stimulatory cytokine.

Inhibitory checkpoint molecules include but are not limited to PD-1, TIM-3, VISTA, A2AR, B7-H3, B7-H4, BTLA, CTLA-4, IDO, KIR and LAG3. CTLA-4, PD-1 and its ligands are members of the CD28-B7 family of co-signaling molecules that play important roles throughout all stages of T-cell function and other cell functions. CTLA-4, Cytotoxic T-Lymphocyte-Associated protein 4 (CD152), is involved in controlling T cell proliferation.

The PD-1 receptor is expressed on the surface of activated T cells (and B cells) and, under normal circumstances, binds to its ligands (PD-L1 and PD-L2) that are expressed on the surface of antigen-presenting cells, such as dendritic cells or macrophages. This interaction sends a signal into the T cell and inhibits it. Cancer cells take advantage of this system by driving high levels of expression of PD-L1 on their surface. This allows them to gain control of the PD-1 pathway and switch off T cells expressing PD-1 that may enter the tumor microenvironment, thus suppressing the anticancer immune response. Pembrolizumab (formerly MK-3475 and lambrolizumab, trade name Keytruda) is a human antibody used in cancer immunotherapy. It targets the PD-1 receptor.

IDO, Indoleamine 2,3-dioxygenase, is a tryptophan catabolic enzyme, which suppresses T and NK cells, generates and activates Tregs and myeloid-derived suppressor cells, and promotes tumor angiogenesis. TIM-3, T-cell Immunoglobulin domain and Mucin domain 3, acts as a negative regulator of Th1/Tc1 function by triggering cell death upon interaction with its ligand, galectin-9. VISTA, V-domain Ig suppressor of T cell activation.

The checkpoint inhibitor is a molecule such as a monoclonal antibody, a humanized antibody, a fully human antibody, a fusion protein or a combination thereof or a small molecule. For instance, the checkpoint inhibitor inhibits a checkpoint protein which may be CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. Ligands of checkpoint proteins include but are not limited to CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands. In some embodiments the anti-PD-1 antibody is BMS-936558 (nivolumab). In other embodiments the anti-CTLA-4 antibody is ipilimumab (trade name Yervoy, formerly known as MDX-010 and MDX-101).

In some preferred embodiments the cancer therapeutic agents, including the checkpoint modulators, are delivered in the form of mRNA encoding the cancer therapeutic agents, e.g., anti-PD1, cytokines, chemokines or stimulatory receptors/ligands (e.g., OX40.

In some embodiments the cancer therapeutic agent is a targeted therapy. The targeted therapy may be a BRAF inhibitor such as vemurafenib (PLX4032) or dabrafenib. The BRAF inhibitor may be PLX 4032, PLX 4720, PLX 4734, GDC-0879, PLX 4032, PLX-4720, PLX 4734 and Sorafenib Tosylate. BRAF is a human gene that makes a protein called B-Raf, also referred to as proto-oncogene B-Raf and v-Raf murine sarcoma viral oncogene homolog B1. The B-Raf protein is involved in sending signals inside cells, which are involved in directing cell growth. Vemurafenib, a BRAF inhibitor, was approved by FDA for treatment of late-stage melanoma.

The T-cell therapeutic agent in other embodiments is OX40L. OX40 is a member of the tumor necrosis factor/nerve growth factor receptor (TNFR/NGFR) family. OX40 may play a role in T-cell activation as well as regulation of differentiation, proliferation or apoptosis of normal and malignant lymphoid cells.

In one aspect, the methods of the invention further comprise administering a PD-1 antagonist to the subject. In some aspects, the PD-1 antagonist is an antibody or an antigen-binding portion thereof that specifically binds to PD-1. In a particular aspect, the PD-1 antagonist is a monoclonal antibody. In some aspects, the PD-1 antagonist is selected from the group consisting of Nivolumab, Pembrolizumab, Pidilizumab, and any combination thereof.

In another aspect, the methods of the invention further comprise administering a PDL-1 antagonist to the subject. In some aspects, the PD-L1 antagonist is an antibody or an antigen-binding portion thereof that specifically binds to PD-L1. In a particular aspect, the PD-L1 antagonist is a monoclonal antibody. In some aspects, the PD-L1 antagonist is selected from the group consisting of Durvalumab, Avelumab, MED1473, BMS-936559, Atezolizumab, and any combination thereof.

In another aspect, the methods of the invention further comprise administering a CTLA-4 antagonist to the subject. In some aspects, the CTLA-4 antagonist is an antibody or an antigen-binding portion thereof that specifically binds to CTLA-4. In a particular aspect, the CTLA-4 antagonist is a monoclonal antibody. In some aspects, the CTLA-4 antagonist is selected from the group consisting of Ipilimumab, Tremelimumab, and any combination thereof.

Certain embodiments of the invention provide for a method of treating cancer in a subject in need thereof comprising administering a polynucleotide, in particular, a mRNA encoding a KRAS vaccine peptide with one or more anti-cancer agents to the subject. In some embodiments, the one or more anti-cancer agents is a checkpoint inhibitor antibody or antibodies. In some embodiments, the one or more anti-cancer agents are an mRNA encoding a checkpoint inhibitor antibody or antibodies.

In one aspect, the subject has been previously treated with a PD-1 antagonist prior to the polynucleotide of the present disclosure. In another aspect, the subject has been treated with a monoclonal antibody that binds to PD-1 prior to the polynucleotide of the present disclosure. In another aspect, the subject has been treated with an anti-PD-1 monoclonal antibody therapy prior to the polynucleotide of the present methods. In other aspects, the anti-PD-1 monoclonal antibody therapy comprises Nivolumab, Pembrolizumab, Pidilizumab, or any combination thereof.

In another aspect, the subject has been treated with a monoclonal antibody that binds to PDL-1 prior to the polynucleotide of the present disclosure. In another aspect, the subject has been treated with an anti-PDL-1 monoclonal antibody therapy prior to the polynucleotide of the present methods. In other aspects, the anti-PDL-1 monoclonal antibody therapy comprises Durvalumab, Avelumab, MEDI473, BMS-936559, Atezolizumab, or any combination thereof.

In some aspects, the subject has been treated with a CTLA-4 antagonist prior to the polynucleotide of the present disclosure. In another aspect, the subject has been previously treated with a monoclonal antibody that binds to CTLA-4 prior to the polynucleotide of the present disclosure. In another aspect, the subject has been treated with an anti-CTLA-4 monoclonal antibody prior to the polynucleotide of the present invention. In other aspects, the anti-CTLA-4 antibody therapy comprises Ipilimumab or Tremelimumab.

In one embodiment, the anti-PD-1 antibody (or an antigen-binding portion thereof) useful for the disclosure is pembrolizumab. Pembrolizumab (also known as “KEYTRUDA®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. No. 8,900,587; see also http://www.cancer.gov/drugdictionary?cdrid=695789 (last accessed: Dec. 14, 2014). Pembrolizumab has been approved by the FDA for the treatment of relapsed or refractory melanoma and advanced NSCLC.

In another embodiment, the anti-PD-1 antibody useful for the disclosure is nivolumab. Nivolumab (also known as “OPDIVO®”; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56). Nivolumab has shown activity in a variety of advanced solid tumors including renal cell carcinoma (renal adenocarcinoma, or hypernephroma), melanoma, and non-small cell lung cancer (NSCLC) (Topalian et al., 2012a; Topalian et al., 2014; Drake et al., 2013; WO 2013/173223.

In other embodiments, the anti-PD-1 antibody is MEDI0680 (formerly AMP-514), which is a monoclonal antibody against the PD-1 receptor. MEDI0680 is described, for example, in U.S. Pat. No. 8,609,089B2 or in http://www.cancer.gov/drugdictionary?cdrid=756047 (last accessed Dec. 14, 2014).

In certain embodiments, the anti-PD-1 antibody is BGB-A317, which is a humanized monoclonal antibody. BGB-A317 is described in U.S. Publ. No. 2015/0079109.

In certain embodiments, a PD-1 antagonist is AMP-224, which is a B7-DC Fc fusion protein. AMP-224 is discussed in U.S. Publ. No. 2013/0017199 or in http://www.cancer.gov/publications/dictionaries/cancer-drug?cdrid=700595 (last accessed Jul. 8, 2015).

In certain embodiments, the anti-PD-L1 antibody useful for the disclosure is MSB0010718C (also called Avelumab; See US 2014/0341917) or BMS-936559 (formerly 12A4 or MDX-1105) (see, e.g., U.S. Pat. No. 7,943,743; WO 2013/173223). In other embodiments, the anti-PD-L1 antibody is MPDL3280A (also known as RG7446) (see, e.g., Herbst et al. (2013) J Clin Oncol 31(suppl):3000. Abstract; U.S. Pat. No. 8,217,149), MEDI4736 (also called Durvalumab; Khleif (2013) In: Proceedings from the European Cancer Congress 2013; Sep. 27-Oct. 1, 2013; Amsterdam, The Netherlands.

An exemplary clinical anti-CTLA-4 antibody is the human mAb 10D1 (now known as ipilimumab and marketed as YERVOY®) as disclosed in U.S. Pat. No. 6,984,720. Another anti-CTLA-4 antibody useful for the present methods is tremelimumab (also known as CP-675,206). Tremelimumab is human IgG2 monoclonal anti-CTLA-4 antibody. Tremelimumab is described in WO/2012/122444, U.S. Publ. No. 2012/263677, or WO Publ. No. 2007/113648 A2.

The following Table (Table 10) provides examples of KRAS mutations in specific tumor types and types of therapies in use and testing. The compositions of the invention are useful in combination with any of these therapies.

TABLE 10

| | | | | Uterine endometrioid |
|---|---|---|---|---|
| | Colorectal | Pancreatic | Lung | carcinoma |
| #US KRAS* | 57,712 | 49,257 | 26,695 | 10,281 |
| Patients | | | | |
| (mKRAS Incidence) | | | | |
| % KRAS mutation | 45.0% | 97.0% | 31.0% | 21.4% |
| (vs. Total) | | | | |
| PD-L1 Inhibitors | Atezolizumab | Durvalumab | Avelumab | No |
| tested | (P3-NR) | (P2-R) | (P3-R) | |
| | Durvalumab | | Atezolizumab | |
| | (P2-NR) | | (P3-R) | |
| | | | Durvalumab | |
| | | | (P2-R) | |
| PD-1 Inhibitors | Nivolumab | Nivolumab | Nivolumab | Nivolumab |
| tested | (P2-R) | (P2-R) | (P2-R) | (P2-R) |
| | Pembrolizumab | Pembrolizumab | Pembrolizumab | Pembrolizumab |
| | (P2-R) | (P2-R) | (P2-R) | (P2-R) |
| Cancer Vaccine | No | No | GI-4000 | No |
| tested | | | (P2-C) | |
| | | | DPV-001 | |
| | | | (P2-R) | |
| KRAS Vaccine | No | No | GI-4000 | No |
| tested | | | (P2-C) | |
| | | | DPV-001 | |
| | | | (P2-R) | |
| Bull Case for KRAS | 45% w/mutant | 97% w/mutant | 31% w/mutant | 21% w/mutant KRAS |
| Vaccine | KRAS | KRAS | KRAS | |
| | Largest pt pool | Defines this tumor | 39% G12C allele | |
| | 36% G12D allele | 39% G12D Allele | 21% G12V allele | |
| | 21% G12V allele | 30% G12V Allele | | |
| Priority for KRAS | H | H | H | M |
| Vaccine (H/M/L) | | | | |

In other embodiments the cancer therapeutic agent is a cytokine. In yet other embodiments the cancer therapeutic agent is a vaccine comprising a population based tumor specific antigen.

In other embodiments, the cancer therapeutic agent is vaccine containing one or more traditional antigens expressed by cancer-germline genes (antigens common to tumors found in multiple patients, also referred to as “shared cancer antigens”). In some embodiments, a traditional antigen is one that is known to be found in cancers or tumors generally or in a specific type of cancer or tumor. In some embodiments, a traditional cancer antigen is a non-mutated tumor antigen. In some embodiments, a traditional cancer antigen is a mutated tumor antigen.

The p53 gene (official symbol TP53) is mutated more frequently than any other gene in human cancers. Large cohort studies have shown that, for most p53 mutations, the genomic position is unique to one or only a few patients and the mutation cannot be used as recurrent neoantigens for therapeutic vaccines designed for a specific population of patients. A small subset of p53 loci do, however, exhibit a “hotspot” pattern, in which several positions in the gene are mutated with relatively high frequency. Strikingly, a large portion of these recurrently mutated regions occur near exon-intron boundaries, disrupting the canonical nucleotide sequence motifs recognized by the mRNA splicing machinery.

Mutation of a splicing motif can alter the final mRNA sequence even if no change to the local amino acid sequence is predicted (i.e. for synonymous or intronic mutations). Therefore, these mutations are often annotated as “noncoding” by common annotation tools and neglected for further analysis, even though they may alter mRNA splicing in unpredictable ways and exert severe functional impact on the translated protein. If an alternatively spliced isoform produces an in-frame sequence change (i.e., no pretermination codon (PTC) is produced), it can escape depletion by nonsense-mediated mRNA decay (NMD) and be readily expressed, processed, and presented on the cell surface by the HLA system. Further, mutation-derived alternative splicing is usually “cryptic”, i.e., not expressed in normal tissues, and therefore may be recognized by T-cells as non-self neoantigens.

In some instances, the cancer therapeutic agent is a vaccine which includes one or more neoantigens which are recurrent polymorphisms (“hot spot mutations”). For example, among other things, the present invention provides neoantigen peptide sequences resulting from certain recurrent somatic cancer mutations in p53. Exemplary mutations and mRNA splicing events resulting neoantigen peptides and HLA-restricted epitopes include, but are not limited to the following:

(1) mutations at the canonical 5′ splice site neighboring codon p.T125, inducing a retained intron having peptide sequence TAKSVTCTVSCPEGLASMRLQCLAVSPCISFVWNFGIPLHPLASCQCFFIVYPLNV (SEQ ID NO: 232) that contains epitopes AVSPCISFVW (SEQ ID NO: 233) (HLA-B*57:01, HLA-B*58:01), HPLASCQCFF (SEQ ID NO: 234) (HLA-B*35:01, HLA-B*53:01), FVWNFGIPL (SEQ ID NO: 235) (HLA-A*02:01, HLA-A*02:06, HLA-B*35:01);

(2) mutations at the canonical 5′ splice site neighboring codon p.331, inducing a retained intron having peptide sequence EYFTLQVLSLGTSYQVESFQSNTQNAVFFLTVLPAIGAFAIRGQ (SEQ ID NO: 236) that contains epitopes LQVLSLGTSY (SEQ ID NO: 237) (HLA-B*15:01), FQSNTQNAVF (SEQ ID NO: 238) (HLA-B*15:01);

(3) mutations at the canonical 3′ splice site neighboring codon p.126, inducing a cryptic alternative exonic 3′ splice site producing the novel spanning peptide sequence AKSVTCTMFCQLAK (SEQ ID NO: 239) that contains epitopes CTMFCQLAK (SEQ ID NO: 240) (HLA-A*11:01), KSVTCTMF (SEQ ID NO: 241) (HLA-B*58:01); and/or

(4) mutations at the canonical 5′ splice site neighboring codon p.224, inducing a cryptic alternative intronic 5′ splice site producing the novel spanning peptide sequence VPYEPPEVWLALTVPPSTAWAA (SEQ ID NO: 242) that contains epitopes VPYEPPEVW (SEQ ID NO: 243) (HLA-B*53:01, HLA-B*51:01), LTVPPSTAW (SEQ ID NO: 244) (HLA-B*58:01, HLA-B*57:01),

wherein the transcript codon positions refer to the canonical full-length p53 transcript ENST00000269305 (SEQ ID NO: 245) from the Ensembl v83 human genome annotation.

In one embodiment, the invention provides a cancer therapeutic vaccine comprising mRNA encoding an open reading frame (ORF) coding for one or more of neoantigen peptides (1) through (4). In one embodiment, the invention provides the selective administration of a vaccine containing or coding for one or more of peptides (1)-(4), based on the patient's tumor containing any of the above mutations. In one embodiment, the invention provides the selective administration of the vaccine based on the dual criteria of the subject's tumor containing any of the above mutations and the subject's normal HLA type containing the corresponding HLA allele predicted to bind to the resulting neoantigen.

In some embodiments, the cancer therapeutic vaccine comprises one or more mRNAs encoding one or more recurrent polymorphisms. In some embodiments, the cancer therapeutic vaccine comprises one or more mRNAs encoding one or more patient specific neoantigens. In some embodiments, the cancer therapeutic vaccine comprises one or more mRNAs encoding an immune checkpoint modulator. The one or more recurrent polymorphisms, the one or more patient specific neoantigens, and/or the one or more immune checkpoint modulator can be combined in any manner. For example, it may desirable for one or more concatameric constructs to encode one the one or more recurrent polymorphisms, the one or more patient specific neoantigens, and/or the one or more immune checkpoint modulator. In other instances, it may be desirable for the one or more recurrent polymorphisms, the one or more patient specific neoantigens, and/or the one or more immune checkpoint modulator to be encoded by separate mRNA constructs. It will be appreciated that the one or more recurrent polymorphisms, the one or more patient specific neoantigens, and/or the one or more immune checkpoint modulator can be administered concurrently, or can be administered sequentially.

The mRNA cancer vaccine and anti-cancer therapeutic can be combined to enhance immune therapeutic responses even further. The mRNA cancer vaccine and other therapeutic agent may be administered simultaneously or sequentially. When the other therapeutic agents are administered simultaneously they can be administered in the same or separate formulations, but are administered at the same time. The other therapeutic agents are administered sequentially with one another and with the mRNA cancer vaccine, when the administration of the other therapeutic agents and the mRNA cancer vaccine is temporally separated. The separation in time between the administration of these compounds may be a matter of minutes or it may be longer, e.g. hours, days, weeks, months. For example, in some embodiments, the separation in time between the administration of these compounds is 1 hour, 2 hours, 3 hours 4 hours, 5 hours, 6 hours, 8 hours, 12 hours, 24 hours or more. In some embodiments, the separation in time between the administration of these compounds is 2 days, 3 days, 4 days, 5 days, 6 days, or 7 days or more. In some embodiments, the mRNA cancer vaccine is administered before the anti-cancer therapeutic. In some embodiments, the mRNA cancer vaccine is administered after the anti-cancer therapeutic.

Other therapeutic agents include but are not limited to anti-cancer therapeutic, adjuvants, cytokines, antibodies, antigens, etc.

In some aspects, provided methods include administering an mRNA cancer vaccine in combination with an immune checkpoint modulator. In some embodiments, an immune checkpoint modulator, e.g., checkpoint inhibitor such as an anti-PD-1 antibody, is administered at a dosage level sufficient to deliver 100-300 mg to the subject. In some embodiments, an immune checkpoint modulator, e.g., checkpoint inhibitor such as an anti-PD-1 antibody, is administered at a dosage level sufficient to deliver 200 mg to the subject. In some embodiments, an immune checkpoint modulator, e.g., checkpoint inhibitor such as an anti-PD-1 antibody, is administered by intravenous infusion. In some embodiments, thee immune checkpoint modulator is administered to the subject twice, three times, four times or more. In some embodiments, the immune checkpoint modulator is administered to the subject on the same day as the mRNA vaccine administration.

RNA vaccines may be formulated or administered in combination with one or more pharmaceutically-acceptable excipients. In some embodiments, vaccine compositions comprise at least one additional active substances, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, cancer RNA vaccines are administered to humans, human patients or subjects. For the purposes of the present disclosure, the phrase “active ingredient” generally refers to the RNA vaccines or the polynucleotides contained therein, for example, RNA polynucleotides (e.g., mRNA polynucleotides) encoding antigenic polypeptides.

Formulations of the vaccine compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

Cancer RNA vaccines can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with cancer RNA vaccines (e.g., for transplantation into a subject), hyaluronidase, nanoparticle mimics and combinations thereof.

### Accelerated Blood Clearance

The invention provides compounds, compositions and methods of use thereof for reducing the effect of ABC on a repeatedly administered active agent such as a biologically active agent. As will be readily apparent, reducing or eliminating altogether the effect of ABC on an administered active agent effectively increases its half-life and thus its efficacy.

In some embodiments the term reducing ABC refers to any reduction in ABC in comparison to a positive reference control ABC inducing LNP such as an MC3 LNP. ABC inducing LNPs cause a reduction in circulating levels of an active agent upon a second or subsequent administration within a given time frame. Thus a reduction in ABC refers to less clearance of circulating agent upon a second or subsequent dose of agent, relative to a standard LNP. The reduction may be, for instance, at least 10%, 15%, 20%, 25%, 30%, 35%, 40%, 45%, 50%, 55%, 60%, 65%, 70%, 75%, 80%, 85%, 90%, 95%, 98%, or 100%. In some embodiments the reduction is 10-100%, 10-50%, 20-100%, 20-50%, 30-100%, 30-50%, 40%-100%, 40-80%, 50-90%, or 50-100%. Alternatively the reduction in ABC may be characterized as at least a detectable level of circulating agent following a second or subsequent administration or at least a 2 fold, 3 fold, 4 fold, 5 fold increase in circulating agent relative to circulating agent following administration of a standard LNP. In some embodiments the reduction is a 2-100 fold, 2-50 fold, 3-100 fold, 3-50 fold, 3-20 fold, 4-100 fold, 4-50 fold, 4-40 fold, 4-30 fold, 4-25 fold, 4-20 fold, 4-15 fold, 4-10 fold, 4-5 fold, 5-100 fold, 5-50 fold, 5-40 fold, 5-30 fold, 5-25 fold, 5-20 fold, 5-15 fold, 5-10 fold, 6-100 fold, 6-50 fold, 6-40 fold, 6-30 fold, 6-25 fold, 6-20 fold, 6-15 fold, 6-10 fold, 8-100 fold, 8-50 fold, 8-40 fold, 8-30 fold, 8-25 fold, 8-20 fold, 8-15 fold, 8-10 fold, 10-100 fold, 10-50 fold, 10-40 fold, 10-30 fold, 10-25 fold, 10-20 fold, 10-15 fold, 20-100 fold, 20-50 fold, 20-40 fold, 20-30 fold, or 20-25 fold.

The disclosure provides lipid-comprising compounds and compositions that are less susceptible to clearance and thus have a longer half-life in vivo. This is particularly the case where the compositions are intended for repeated including chronic administration, and even more particularly where such repeated administration occurs within days or weeks.

Significantly, these compositions are less susceptible or altogether circumvent the observed phenomenon of accelerated blood clearance (ABC). ABC is a phenomenon in which certain exogenously administered agents are rapidly cleared from the blood upon second and subsequent administrations. This phenomenon has been observed, in part, for a variety of lipid-containing compositions including but not limited to lipidated agents, liposomes or other lipid-based delivery vehicles, and lipid-encapsulated agents. Heretofore, the basis of ABC has been poorly understood and in some cases attributed to a humoral immune response and accordingly strategies for limiting its impact in vivo particularly in a clinical setting have remained elusive.

This disclosure provides compounds and compositions that are less susceptible, if at all susceptible, to ABC. In some important aspects, such compounds and compositions are lipid-comprising compounds or compositions. The lipid-containing compounds or compositions of this disclosure, surprisingly, do not experience ABC upon second and subsequent administration in vivo. This resistance to ABC renders these compounds and compositions particularly suitable for repeated use in vivo, including for repeated use within short periods of time, including days or 1-2 weeks. This enhanced stability and/or half-life is due, in part, to the inability of these compositions to activate B1a and/or B1b cells and/or conventional B cells, pDCs and/or platelets.

This disclosure therefore provides an elucidation of the mechanism underlying accelerated blood clearance (ABC). It has been found, in accordance with this disclosure and the inventions provided herein, that the ABC phenomenon at least as it relates to lipids and lipid nanoparticles is mediated, at least in part an innate immune response involving B1a and/or B1b cells, pDC and/or platelets. B1a cells are normally responsible for secreting natural antibody, in the form of circulating IgM. This IgM is poly-reactive, meaning that it is able to bind to a variety of antigens, albeit with a relatively low affinity for each.

It has been found in accordance with the invention that some lipidated agents or lipid-comprising formulations such as lipid nanoparticles administered in vivo trigger and are subject to ABC. It has now been found in accordance with the invention that upon administration of a first dose of the LNP, one or more cells involved in generating an innate immune response (referred to herein as sensors) bind such agent, are activated, and then initiate a cascade of immune factors (referred to herein as effectors) that promote ABC and toxicity. For instance, B1a and B1b cells may bind to LNP, become activated (alone or in the presence of other sensors such as pDC and/or effectors such as IL6) and secrete natural IgM that binds to the LNP. Pre-existing natural IgM in the subject may also recognize and bind to the LNP, thereby triggering complement fixation. After administration of the first dose, the production of natural IgM begins within 1-2 hours of administration of the LNP. Typically by about 2-3 weeks the natural IgM is cleared from the system due to the natural half-life of IgM. Natural IgG is produced beginning around 96 hours after administration of the LNP. The agent, when administered in a naïve setting, can exert its biological effects relatively unencumbered by the natural IgM produced post-activation of the B1a cells or B1b cells or natural IgG. The natural IgM and natural IgG are non-specific and thus are distinct from anti-PEG IgM and anti-PEG IgG.

Although Applicant is not bound by mechanism, it is proposed that LNPs trigger ABC and/or toxicity through the following mechanisms. It is believed that when an LNP is administered to a subject the LNP is rapidly transported through the blood to the spleen. The LNPs may encounter immune cells in the blood and/or the spleen. A rapid innate immune response is triggered in response to the presence of the LNP within the blood and/or spleen. Applicant has shown herein that within hours of administration of an LNP several immune sensors have reacted to the presence of the LNP. These sensors include but are not limited to immune cells involved in generating an immune response, such as B cells, pDC, and platelets. The sensors may be present in the spleen, such as in the marginal zone of the spleen and/or in the blood. The LNP may physically interact with one or more sensors, which may interact with other sensors. In such a case the LNP is directly or indirectly interacting with the sensors. The sensors may interact directly with one another in response to recognition of the LNP. For instance many sensors are located in the spleen and can easily interact with one another. Alternatively one or more of the sensors may interact with LNP in the blood and become activated. The activated sensor may then interact directly with other sensors or indirectly (e.g., through the stimulation or production of a messenger such as a cytokine e.g., 1L6).

In some embodiments the LNP may interact directly with and activate each of the following sensors: pDC, B1a cells, B1b cells, and platelets. These cells may then interact directly or indirectly with one another to initiate the production of effectors which ultimately lead to the ABC and/or toxicity associated with repeated doses of LNP. For instance, Applicant has shown that LNP administration leads to pDC activation, platelet aggregation and activation and B cell activation. In response to LNP platelets also aggregate and are activated and aggregate with B cells. pDC cells are activated. LNP has been found to interact with the surface of platelets and B cells relatively quickly. Blocking the activation of any one or combination of these sensors in response to LNP is useful for dampening the immune response that would ordinarily occur. This dampening of the immune response results in the avoidance of ABC and/or toxicity.

The sensors once activated produce effectors. An effector, as used herein, is an immune molecule produced by an immune cell, such as a B cell. Effectors include but are not limited to immunoglobulin such as natural IgM and natural IgG and cytokines such as IL6. B1a and B1b cells stimulate the production of natural IgMs within 2-6 hours following administration of an LNP. Natural IgG can be detected within 96 hours. L6 levels are increased within several hours. The natural IgM and IgG circulate in the body for several days to several weeks. During this time the circulating effectors can interact with newly administered LNPs, triggering those LNPs for clearance by the body. For instance, an effector may recognize and bind to an LNP. The Fc region of the effector may be recognized by and trigger uptake of the decorated LNP by macrophage. The macrophage are then transported to the spleen. The production of effectors by immune sensors is a transient response that correlates with the timing observed for ABC.

If the administered dose is the second or subsequent administered dose, and if such second or subsequent dose is administered before the previously induced natural IgM and/or IgG is cleared from the system (e.g., before the 2-3 window time period), then such second or subsequent dose is targeted by the circulating natural IgM and/or natural IgG or Fc which trigger alternative complement pathway activation and is itself rapidly cleared. When LNP are administered after the effectors have cleared from the body or are reduced in number, ABC is not observed.

Thus, it is useful according to aspects of the invention to inhibit the interaction between LNP and one or more sensors, to inhibit the activation of one or more sensors by LNP (direct or indirect), to inhibit the production of one or more effectors, and/or to inhibit the activity of one or more effectors. In some embodiments the LNP is designed to limit or block interaction of the LNP with a sensor. For instance the LNP may have an altered PC and/or PEG to prevent interactions with sensors. Alternatively or additionally an agent that inhibits immune responses induced by LNPs may be used to achieve any one or more of these effects.

It has also been determined that conventional B cells are also implicated in ABC. Specifically, upon first administration of an agent, conventional B cells, referred to herein as CD 19(+), bind to and react against the agent. Unlike B1a and B1b cells though, conventional B cells are able to mount first an IgM response (beginning around 96 hours after administration of the LNPs) followed by an IgG response (beginning around 14 days after administration of the LNPs) concomitant with a memory response. Thus conventional B cells react against the administered agent and contribute to IgM (and eventually IgG) that mediates ABC. The IgM and IgG are typically anti-PEG IgM and anti-PEG IgG.

It is contemplated that in some instances, the majority of the ABC response is mediated through B1a cells and B1a-mediated immune responses. It is further contemplated that in some instances, the ABC response is mediated by both IgM and IgG, with both conventional B cells and B1a cells mediating such effects. In yet still other instances, the ABC response is mediated by natural IgM molecules, some of which are capable of binding to natural IgM, which may be produced by activated B1a cells. The natural IgMs may bind to one or more components of the LNPs, e.g., binding to a phospholipid component of the LNPs (such as binding to the PC moiety of the phospholipid) and/or binding to a PEG-lipid component of the LNPs (such as binding to PEG-DMG, in particular, binding to the PEG moiety of PEG-DMG). Since B1a expresses CD36, to which phosphatidylcholine is a ligand, it is contemplated that the CD36 receptor may mediate the activation of B1a cells and thus production of natural IgM. In yet still other instances, the ABC response is mediated primarily by conventional B cells.

It has been found in accordance with the invention that the ABC phenomenon can be reduced or abrogated, at least in part, through the use of compounds and compositions (such as agents, delivery vehicles, and formulations) that do not activate B1a cells. Compounds and compositions that do not activate B1a cells may be referred to herein as B1a inert compounds and compositions. It has been further found in accordance with the invention that the ABC phenomenon can be reduced or abrogated, at least in part, through the use of compounds and compositions that do not activate conventional B cells. Compounds and compositions that do not activate conventional B cells may in some embodiments be referred to herein as CD19-inert compounds and compositions. Thus, in some embodiments provided herein, the compounds and compositions do not activate B1a cells and they do not activate conventional B cells. Compounds and compositions that do not activate B1a cells and conventional B cells may in some embodiments be referred to herein as B1a/CD19-inert compounds and compositions.

These underlying mechanisms were not heretofore understood, and the role of B1a and B1b cells and their interplay with conventional B cells in this phenomenon was also not appreciated.

Accordingly, this disclosure provides compounds and compositions that do not promote ABC. These may be further characterized as not capable of activating B1a and/or B1b cells, platelets and/or pDC, and optionally conventional B cells also. These compounds (e.g., agents, including biologically active agents such as prophylactic agents, therapeutic agents and diagnostic agents, delivery vehicles, including liposomes, lipid nanoparticles, and other lipid-based encapsulating structures, etc.) and compositions (e.g., formulations, etc.) are particularly desirable for applications requiring repeated administration, and in particular repeated administrations that occur within with short periods of time (e.g., within 1-2 weeks). This is the case, for example, if the agent is a nucleic acid based therapeutic that is provided to a subject at regular, closely-spaced intervals. The findings provided herein may be applied to these and other agents that are similarly administered and/or that are subject to ABC.

Of particular interest are lipid-comprising compounds, lipid-comprising particles, and lipid-comprising compositions as these are known to be susceptible to ABC. Such lipid-comprising compounds particles, and compositions have been used extensively as biologically active agents or as delivery vehicles for such agents. Thus, the ability to improve their efficacy of such agents, whether by reducing the effect of ABC on the agent itself or on its delivery vehicle, is beneficial for a wide variety of active agents.

Also provided herein are compositions that do not stimulate or boost an acute phase response (ARP) associated with repeat dose administration of one or more biologically active agents.

The composition, in some instances, may not bind to IgM, including but not limited to natural IgM.

The composition, in some instances, may not bind to an acute phase protein such as but not limited to C-reactive protein.

The composition, in some instances, may not trigger a CD5(+) mediated immune response. As used herein, a CD5(+) mediated immune response is an immune response that is mediated by B1a and/or B1b cells. Such a response may include an ABC response, an acute phase response, induction of natural IgM and/or IgG, and the like.

The composition, in some instances, may not trigger a CD19(+) mediated immune response. As used herein, a CD19(+) mediated immune response is an immune response that is mediated by conventional CD19(+), CD5(−) B cells. Such a response may include induction of IgM, induction of IgG, induction of memory B cells, an ABC response, an anti-drug antibody (ADA) response including an anti-protein response where the protein may be encapsulated within an LNP, and the like.

B1a cells are a subset of B cells involved in innate immunity. These cells are the source of circulating IgM, referred to as natural antibody or natural serum antibody. Natural IgM antibodies are characterized as having weak affinity for a number of antigens, and therefore they are referred to as “poly-specific” or “poly-reactive”, indicating their ability to bind to more than one antigen. B1a cells are not able to produce IgG. Additionally, they do not develop into memory cells and thus do not contribute to an adaptive immune response. However, they are able to secrete IgM upon activation. The secreted IgM is typically cleared within about 2-3 weeks, at which point the immune system is rendered relatively naïve to the previously administered antigen. If the same antigen is presented after this time period (e.g., at about 3 weeks after the initial exposure), the antigen is not rapidly cleared. However, significantly, if the antigen is presented within that time period (e.g., within 2 weeks, including within 1 week, or within days), then the antigen is rapidly cleared. This delay between consecutive doses has rendered certain lipid-containing therapeutic or diagnostic agents unsuitable for use.

In humans, B1a cells are CD19(+), CD20(+), CD27(+), CD43(+), CD70(−) and CD5(+). In mice, B1a cells are CD19(+), CD5(+), and CD45 B cell isoform B220(+). It is the expression of CD5 which typically distinguishes B1a cells from other convention B cells. B1a cells may express high levels of CD5, and on this basis may be distinguished from other B-1 cells such as B-1b cells which express low or undetectable levels of CD5. CD5 is a pan-T cell surface glycoprotein. B1a cells also express CD36, also known as fatty acid translocase. CD36 is a member of the class B scavenger receptor family. CD36 can bind many ligands, including oxidized low density lipoproteins, native lipoproteins, oxidized phospholipids, and long-chain fatty acids.

B1b cells are another subset of B cells involved in innate immunity. These cells are another source of circulating natural IgM. Several antigens, including PS, are capable of inducing T cell independent immunity through B1b activation. CD27 is typically upregulated on B1b cells in response to antigen activation. Similar to B1a cells, the B1b cells are typically located in specific body locations such as the spleen and peritoneal cavity and are in very low abundance in the blood. The B1b secreted natural IgM is typically cleared within about 2-3 weeks, at which point the immune system is rendered relatively naïve to the previously administered antigen. If the same antigen is presented after this time period (e.g., at about 3 weeks after the initial exposure), the antigen is not rapidly cleared. However, significantly, if the antigen is presented within that time period (e.g., within 2 weeks, including within 1 week, or within days), then the antigen is rapidly cleared. This delay between consecutive doses has rendered certain lipid-containing therapeutic or diagnostic agents unsuitable for use.

In some embodiments it is desirable to block B a and/or B1b cell activation. One strategy for blocking B1a and/or B1b cell activation involves determining which components of a lipid nanoparticle promote B cell activation and neutralizing those components. It has been discovered herein that at least PEG and phosphatidylcholine (PC) contribute to B1a and B1b cell interaction with other cells and/or activation. PEG may play a role in promoting aggregation between B1 cells and platelets, which may lead to activation. PC (a helper lipid in LNPs) is also involved in activating the B1 cells, likely through interaction with the CD36 receptor on the B cell surface. Numerous particles have PEG-lipid alternatives, PEG-less, and/or PC replacement lipids (e.g. oleic acid or analogs thereof) have been designed and tested. Applicant has established that replacement of one or more of these components within an LNP that otherwise would promote ABC upon repeat administration, is useful in preventing ABC by reducing the production of natural IgM and/or B cell activation. Thus, the invention encompasses LNPs that have reduced ABC as a result of a design which eliminates the inclusion of B cell triggers.

Another strategy for blocking B1a and/or B1b cell activation involves using an agent that inhibits immune responses induced by LNPs. These types of agents are discussed in more detail below. In some embodiments these agents block the interaction between B1a/B1b cells and the LNP or platelets or pDC. For instance the agent may be an antibody or other binding agent that physically blocks the interaction. An example of this is an antibody that binds to CD36 or CD6. The agent may also be a compound that prevents or disables the B1a/B1b cell from signaling once activated or prior to activation. For instance, it is possible to block one or more components in the B1a/B1b signaling cascade the results from B cell interaction with LNP or other immune cells. In other embodiments the agent may act one or more effectors produced by the B1a/B1b cells following activation. These effectors include for instance, natural IgM and cytokines.

It has been demonstrated according to aspects of the invention that when activation of pDC cells is blocked, B cell activation in response to LNP is decreased. Thus, in order to avoid ABC and/or toxicity, it may be desirable to prevent pDC activation. Similar to the strategies discussed above, pDC cell activation may be blocked by agents that interfere with the interaction between pDC and LNP and/or B cells/platelets. Alternatively agents that act on the pDC to block its ability to get activated or on its effectors can be used together with the LNP to avoid ABC.

Platelets may also play an important role in ABC and toxicity. Very quickly after a first dose of LNP is administered to a subject platelets associate with the LNP, aggregate and are activated. In some embodiments it is desirable to block platelet aggregation and/or activation. One strategy for blocking platelet aggregation and/or activation involves determining which components of a lipid nanoparticle promote platelet aggregation and/or activation and neutralizing those components. It has been discovered herein that at least PEG contribute to platelet aggregation, activation and/or interaction with other cells. Numerous particles have PEG-lipid alternatives and PEG-less have been designed and tested. Applicant has established that replacement of one or more of these components within an LNP that otherwise would promote ABC upon repeat administration, is useful in preventing ABC by reducing the production of natural IgM and/or platelet aggregation. Thus, the invention encompasses LNPs that have reduced ABC as a result of a design which eliminates the inclusion of platelet triggers. Alternatively agents that act on the platelets to block its activity once it is activated or on its effectors can be used together with the LNP to avoid ABC.

### Measuring ABC Activity and Related Activities

Various compounds and compositions provided herein, including LNPs, do not promote ABC activity upon administration in vivo. These LNPs may be characterized and/or identified through any of a number of assays, such as but not limited to those described below.

In some embodiments the methods involve administering an LNP without producing an immune response that promotes ABC. An immune response that promotes ABC involves activation of one or more sensors, such as B1 cells, pDC, or platelets, and one or more effectors, such as natural IgM, natural IgG or cytokines such as IL6. Thus administration of an LNP without producing an immune response that promotes ABC, at a minimum involves administration of an LNP without significant activation of one or more sensors and significant production of one or more effectors. Significant used in this context refers to an amount that would lead to the physiological consequence of accelerated blood clearance of all or part of a second dose with respect to the level of blood clearance expected for a second dose of an ABC triggering LNP. For instance, the immune response should be dampened such that the ABC observed after the second dose is lower than would have been expected for an ABC triggering LNP.

### B1a or B1b Activation Assay

Certain compositions provided in this disclosure do not activate B cells, such as B1a or B1b cells (CD19+ CD5+) and/or conventional B cells (CD19+ CD5−). Activation of B1a cells, B1b cells, or conventional B cells may be determined in a number of ways, some of which are provided below. B cell population may be provided as fractionated B cell populations or unfractionated populations of splenocytes or peripheral blood mononuclear cells (PBMC). If the latter, the cell population may be incubated with the LNP of choice for a period of time, and then harvested for further analysis. Alternatively, the supernatant may be harvested and analyzed.

### Upregulation of Activation Marker Cell Surface Expression

Activation of B1a cells, B1b cells, or conventional B cells may be demonstrated as increased expression of B cell activation markers including late activation markers such as CD86. In an exemplary non-limiting assay, unfractionated B cells are provided as a splenocyte population or as a PBMC population, incubated with an LNP of choice for a particular period of time, and then stained for a standard B cell marker such as CD19 and for an activation marker such as CD86, and analyzed using for example flow cytometry. A suitable negative control involves incubating the same population with medium, and then performing the same staining and visualization steps. An increase in CD86 expression in the test population compared to the negative control indicates B cell activation.

### Pro-Inflammatory Cytokine Release

B cell activation may also be assessed by cytokine release assay. For example, activation may be assessed through the production and/or secretion of cytokines such as IL-6 and/or TNF-alpha upon exposure with LNPs of interest.

Such assays may be performed using routine cytokine secretion assays well known in the art. An increase in cytokine secretion is indicative of B cell activation.

LNP Binding/Association to and/or Uptake by B Cells

LNP association or binding to B cells may also be used to assess an LNP of interest and to further characterize such LNP. Association/binding and/or uptake/internalization may be assessed using a detectably labeled, such as fluorescently labeled, LNP and tracking the location of such LNP in or on B cells following various periods of incubation.

The invention further contemplates that the compositions provided herein may be capable of evading recognition or detection and optionally binding by downstream mediators of ABC such as circulating IgM and/or acute phase response mediators such as acute phase proteins (e.g., C-reactive protein (CRP).

### Methods of Use for Reducing ABC

Also provided herein are methods for delivering LNPs, which may encapsulate an agent such as a therapeutic agent, to a subject without promoting ABC.

In some embodiments, the method comprises administering any of the LNPs described herein, which do not promote ABC, for example, do not induce production of natural IgM binding to the LNPs, do not activate B1a and/or B1b cells. As used herein, an LNP that “does not promote ABC” refers to an LNP that induces no immune responses that would lead to substantial ABC or a substantially low level of immune responses that is not sufficient to lead to substantial ABC. An LNP that does not induce the production of natural IgMs binding to the LNP refers to LNPs that induce either no natural IgM binding to the LNPs or a substantially low level of the natural IgM molecules, which is insufficient to lead to substantial ABC. An LNP that does not activate B1a and/or B1b cells refer to LNPs that induce no response of B1a and/or B1b cells to produce natural IgM binding to the LNPs or a substantially low level of B1a and/or B1b responses, which is insufficient to lead to substantial ABC.

In some embodiments the terms do not activate and do not induce production are a relative reduction to a reference value or condition. In some embodiments the reference value or condition is the amount of activation or induction of production of a molecule such as IgM by a standard LNP such as an MC3 LNP. In some embodiments the relative reduction is a reduction of at least 30%, for example at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100%. In other embodiments the terms do not activate cells such as B cells and do not induce production of a protein such as IgM may refer to an undetectable amount of the active cells or the specific protein.

### Platelet Effects and Toxicity

The invention is further premised in part on the elucidation of the mechanism underlying dose-limiting toxicity associated with LNP administration. Such toxicity may involve coagulopathy, disseminated intravascular coagulation (DIC, also referred to as consumptive coagulopathy), whether acute or chronic, and/or vascular thrombosis. In some instances, the dose-limiting toxicity associated with LNPs is acute phase response (APR) or complement activation-related psudoallergy (CARPA).

As used herein, coagulopathy refers to increased coagulation (blood clotting) in vivo. The findings reported in this disclosure are consistent with such increased coagulation and significantly provide insight on the underlying mechanism. Coagulation is a process that involves a number of different factors and cell types, and heretofore the relationship between and interaction of LNPs and platelets has not been understood in this regard. This disclosure provides evidence of such interaction and also provides compounds and compositions that are modified to have reduced platelet effect, including reduced platelet association, reduced platelet aggregation, and/or reduced platelet aggregation. The ability to modulate, including preferably down-modulate, such platelet effects can reduce the incidence and/or severity of coagulopathy post-LNP administration. This in turn will reduce toxicity relating to such LNP, thereby allowing higher doses of LNPs and importantly their cargo to be administered to patients in need thereof.

CARPA is a class of acute immune toxicity manifested in hypersensitivity reactions (HSRs), which may be triggered by nanomedicines and biologicals. Unlike allergic reactions, CARPA typically does not involve IgE but arises as a consequence of activation of the complement system, which is part of the innate immune system that enhances the body's abilities to clear pathogens. One or more of the following pathways, the classical complement pathway (CP), the alternative pathway (AP), and the lectin pathway (LP), may be involved in CARPA. Szebeni, Molecular Immunology, 61:163-173 (2014).

The classical pathway is triggered by activation of the C1-complex, which contains. C1q, C₁r, C1s, or C1qr2s2. Activation of the C1-complex occurs when C1q binds to IgM or IgG complexed with antigens, or when C1q binds directly to the surface of the pathogen. Such binding leads to conformational changes in the C1q molecule, which leads to the activation of C1r, which in turn, cleave C1s. The C1r2s2 component now splits C4 and then C2, producing C4a, C4b, C2a, and C2b. C4b and C2b bind to form the classical pathway C3-convertase (C4b2b complex), which promotes cleavage of C3 into C3a and C3b. C3b then binds the C3 convertase to from the C5 convertase (C4b2b3b complex). The alternative pathway is continuously activated as a result of spontaneous C3 hydrolysis. Factor P (properdin) is a positive regulator of the alternative pathway. Oligomerization of properdin stabilizes the C3 convertase, which can then cleave much more C3. The C3 molecules can bind to surfaces and recruit more B, D, and P activity, leading to amplification of the complement activation.

Acute phase response (APR) is a complex systemic innate immune responses for preventing infection and clearing potential pathogens. Numerous proteins are involved in APR and C-reactive protein is a well-characterized one.

It has been found, in accordance with the invention, that certain LNP are able to associate physically with platelets almost immediately after administration in vivo, while other LNP do not associate with platelets at all or only at background levels. Significantly, those LNPs that associate with platelets also apparently stabilize the platelet aggregates that are formed thereafter. Physical contact of the platelets with certain LNPs correlates with the ability of such platelets to remain aggregated or to form aggregates continuously for an extended period of time after administration. Such aggregates comprise activated platelets and also innate immune cells such as macrophages and B cells.

### Lipid Nanoparticles (LNPs)

In one set of embodiments, lipid nanoparticles (LNPs) are provided. In one embodiment, a lipid nanoparticle comprises lipids including an ionizable lipid, a structural lipid, a phospholipid, and mRNA. Each of the LNPs described herein may be used as a formulation for the mRNA described herein. In one embodiment, a lipid nanoparticle comprises an ionizable lipid, a structural lipid, a phospholipid, and mRNA. In some embodiments, the LNP comprises an ionizable lipid, a PEG-modified lipid, a phospholipid and a structural lipid. In some embodiments, the LNP has a molar ratio of about 20-60% ionizable lipid:about 5-25% phospholipid:about 25-55% structural lipid; and about 0.5-15% PEG-modified lipid. In some embodiments, the LNP comprises a molar ratio of about 50% ionizable lipid, about 1.5% PEG-modified lipid, about 38.5% structural lipid and about 10% phospholipid. In some embodiments, the LNP comprises a molar ratio of about 55% ionizable lipid, about 2.5% PEG lipid, about 32.5% structural lipid and about 10% phospholipid. In some embodiments, the ionizable lipid is an ionizable amino or cationic lipid and the phospholipid is a neutral lipid, and the structural lipid is a cholesterol. In some embodiments, the LNP has a molar ratio of 50:38.5:10:1.5 of ionizable lipid:cholesterol:DSPC:PEG2000-DMG.

### Ionizable Amino Lipids

The present disclosure provides pharmaceutical compositions with advantageous properties. For example, the lipids described herein (e.g. those having any of Formula (I), (IA), (II), (IIa), (IIb), (IIc), (IId), (IIe), (III), (IV), (V), or (VI) may be advantageously used in lipid nanoparticle compositions for the delivery of therapeutic and/or prophylactic agents to mammalian cells or organs. For example, the lipids described herein have little or no immunogenicity. For example, the lipid compounds disclosed hereinhave a lower immunogenicity as compared to a reference lipid (e.g., MC3, KC2, or DLinDMA). For example, a formulation comprising a lipid disclosed herein and a therapeutic or prophylactic agent has an increased therapeutic index as compared to a corresponding formulation which comprises a reference lipid (e.g., MC3, KC2, or DLinDMA) and the same therapeutic or prophylactic agent. In particular, the present application provides pharmaceutical compositions comprising:

(a) a polynucleotide comprising a nucleotide sequence encoding one or more cancer epitope polypeptides; and

(b) a delivery agent.

In some embodiments, the delivery agent comprises a lipid compound having the Formula (I)

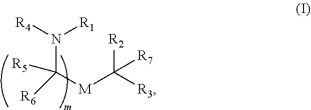

wherein

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;

each R₅ is independently selected from the group consisting of Ca-3 alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₂ alkyl and C₂₋₁₂ alkenyl;

each Y is independently a C₃₋₆ carbocycle;

each X is independently selected from the group consisting of F, Cl, Br, and I; and m is selected from 5, 6, 7, 8, 9, 10, 11, 12, and 13, or salts or stereoisomers thereof.

In some embodiments, a subset of compounds of Formula (I) includes those in which R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and m is selected from 5, 6, 7, 8, 9, 10, 11, 12, and 13,

or salts or stereoisomers thereof, wherein alkyl and alkenyl groups may be linear or branched.

In some embodiments, a subset of compounds of Formula (I) includes those in which when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, and C₁₋₃ alkyl, and each n is independently selected from 1, 2, 3, 4, and 5;

each R₅ is independently selected from the group consisting of C₁₋₃ alkyl, C₂₋₃ alkenyl, and H;

each R6 is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;

each R₅ is independently selected from the group consisting of C₁₋₃ alkyl, C₂₋₃ alkenyl, and H;

each R6 is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and a 5- to 14-membered heterocycloalkyl having one or more heteroatoms selected from N, O, and S which is substituted with one or more substituents selected from oxo (═O), OH, amino, and C₁₋₃ alkyl, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, another subset of compounds of Formula (I) includes those in which

R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, —N(R)₂ and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₈ alkyl, C₂₋₁₈ alkenyl, —R*YR″, —YR, —YR″, and H;

each R″ is independently selected from the group consisting of C₃₋₁₄ alkyl and C₃₋₁₄ alkenyl;

each R* is independently selected from the group consisting of C₁₋₁₂ alkyl, C₁₋₁₂ alkenyl, and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In yet some embodiments, another subset of compounds of Formula (I) includes those in which

R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from —N(R)₂, a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In still some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, -S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In some embodiments, another subset of compounds of Formula (I) includes those in which

R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₁₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In yet some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is —(CH₂)ₙQ or —(CH₂)ₙCHQR, where Q is —N(R)₂, and n is selected from 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₂ alkyl and C₁₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In yet some embodiments, another subset of compounds of Formula (I) includes those in which

R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is —(CH₂)ₙQ or —(CH₂)ₙCHQR, where Q is —N(R)₂, and n is selected from 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In still other embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₂ alkyl and C₁₋₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In still other embodiments, another subset of compounds of Formula (I) includes those in which

R₁ is selected from the group consisting of C₅₋₂₀ alkyl, C₅₋₂₀ alkenyl, —R*YR″, —YR″, and —R″M′R′;

R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In certain embodiments, a subset of compounds of Formula (I) includes those of Formula (IA):

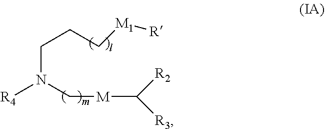

or a salt or stereoisomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl, or heterocycloalkyl; M and M′ are independently selected from —C(O)O—, —OC(O)—, —C(O)N(R′)—, —P(O)(OR′)O—, —S—S—, an aryl group, and a heteroaryl group; and

R₂ and R₃ are independently selected from the group consisting of H, C₁₋₁₄ alkyl, and C₂₋₁₄ alkenyl.

In some embodiments, a subset of compounds of Formula (I) includes those of Formula (IA), Formula (II), or a salt or stereoisomer thereof,

wherein

1 is selected from 1, 2, 3, 4, and 5; m is selected from 5, 6, 7, 8, and 9; M₁ is a bond or M′;

R₄ is unsubstituted C₁₋₃ alkyl, or —(CH₂)ₙQ, in which Q is OH, —NHC(S)N(R)₂, or —NHC(O)N(R)₂;

M and M′ are independently selected from —C(O)O—, —OC(O)—, —C(O)N(R′)—, —P(O)(OR′)O—, an aryl group, and a heteroaryl group; and

R₂ and R₃ are independently selected from the group consisting of H, C₁₋₁₄ alkyl, and C₂₋₁₄ alkenyl.

In certain embodiments, a subset of compounds of Formula (I) includes those of Formula (II):

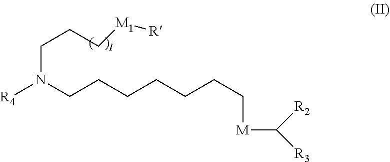

or a salt or stereoisomer thereof, wherein 1 is selected from 1, 2, 3, 4, and 5; M₁ is a bond or M′; R₄ is unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl, or heterocycloalkyl; M and M′ are independently selected from —C(O)O—, —OC(O)—, —C(O)N(R′)—, —P(O)(OR′)O—, —S—S—, an aryl group, and a heteroaryl group; and

R₂ and R₃ are independently selected from the group consisting of H, C₁₋₁₄ alkyl, and C₂₋₁₄ alkenyl.

In some embodiments, a subset of compounds of Formula (I) includes those of Formula (II), or a salt or stereoisomer thereof, wherein

1 is selected from 1, 2, 3, 4, and 5;

M1 is a bond or M′;

R₄ is unsubstituted C₁₋₃ alkyl, or —(CH₂)ₙQ, in which n is 2, 3, or 4, and Q is OH, —NHC(S)N(R)₂, or —NHC(O)N(R)₂;

M and M′ are independently selected from —C(O)O—, —OC(O)—, —C(O)N(R′)—, —P(O)(OR′)O—, an aryl group, and a heteroaryl group; and

R₂ and R₃ are independently selected from the group consisting of H, C₁₋₁₄ alkyl, and C₂₋₁₄ alkenyl.

In some embodiments, the compound of formula (I) is of the formula (IIa),

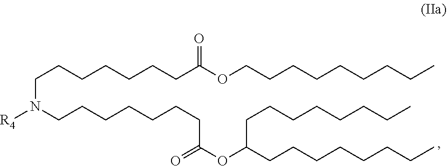

or a salt thereof, wherein R₄ is as described above.

In some embodiments, the compound of formula (I) is of the formula (IIb),

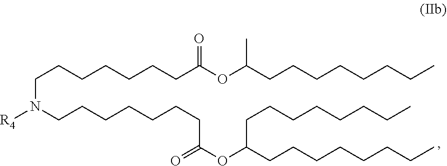

or a salt thereof, wherein R₄ is as described above.

In some embodiments, the compound of formula (I) is of the formula (IIc),

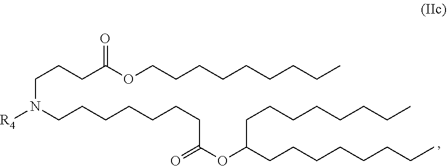

or a salt thereof, wherein R₄ is as described above.

In some embodiments, the compound of formula (I) is of the formula (IIe):

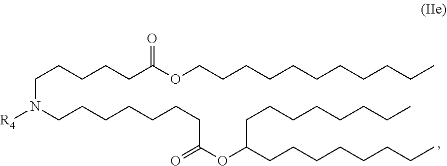

or a salt thereof, wherein R₄ is as described above.

In some embodiments, the compound of formula (IIa), (IIb), (IIc), or (IIe) comprises an R₄ which is selected from —(CH₂)ₙQ and —(CH₂)ₙCHQR, wherein Q, R and n are as defined above.

In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle, wherein R is as defined above. In some aspects, n is 1 or 2. In some embodiments, Q is OH, —NHC(S)N(R)₂, or —NHC(O)N(R)₂.

In some embodiments, the compound of formula (I) is of the formula (IId),

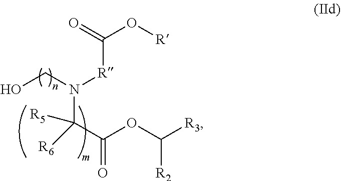

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl, n is selected from 2, 3, and 4, and R′, R″, R₅, R₆ and m are as defined above.

In some aspects of the compound of formula (IId), R₂ is C₈ alkyl. In some aspects of the compound of formula (IId), R₃ is C₅-C₉ alkyl. In some aspects of the compound of formula (IId), m is 5, 7, or 9. In some aspects of the compound of formula (IId), each R₅ is H. In some aspects of the compound of formula (IId), each R₆ is H.

In another aspect, the present application provides a lipid composition (e.g., a lipid nanoparticle (LNP)) comprising: (1) a compound having the formula (I); (2) optionally a helper lipid (e.g. a phospholipid); (3) optionally a structural lipid (e.g. a sterol); and (4) optionally a lipid conjugate (e.g. a PEG-lipid). In exemplary embodiments, the lipid composition (e.g., LNP) further comprises a polynucleotide encoding one or more cancer epitope polypeptides, e.g., a polynucleotide encapsulated therein.

As used herein, the term “alkyl” or “alkyl group” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms).

The notation “C₁₋₁₄ alkyl” means a linear or branched, saturated hydrocarbon including 1-14 carbon atoms. An alkyl group can be optionally substituted.

As used herein, the term “alkenyl” or “alkenyl group” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond.

The notation “C₂₋₁₄ alkenyl” means a linear or branched hydrocarbon including 2-14 carbon atoms and at least one double bond. An alkenyl group can include one, two, three, four, or more double bonds. For example, C₁₈ alkenyl can include one or more double bonds. A C₁₈ alkenyl group including two double bonds can be a linoleyl group. An alkenyl group can be optionally substituted.

As used herein, the term “carbocycle” or “carbocyclic group” means a mono- or multi-cyclic system including one or more rings of carbon atoms. Rings can be three, four, five, six, seven, eight, nine, ten, eleven, twelve, thirteen, fourteen, or fifteen membered rings.

The notation “C₃₋₆ carbocycle” means a carbocycle including a single ring having 3-6 carbon atoms. Carbocycles can include one or more double bonds and can be aromatic (e.g., aryl groups). Examples of carbocycles include cyclopropyl, cyclopentyl, cyclohexyl, phenyl, naphthyl, and 1,2-dihydronaphthyl groups. Carbocycles can be optionally substituted.

As used herein, the term “heterocycle” or “heterocyclic group” means a mono- or multi-cyclic system including one or more rings, where at least one ring includes at least one heteroatom. Heteroatoms can be, for example, nitrogen, oxygen, or sulfur atoms. Rings can be three, four, five, six, seven, eight, nine, ten, eleven, or twelve membered rings. Heterocycles can include one or more double bonds and can be aromatic (e.g., heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. Heterocycles can be optionally substituted.

As used herein, a “biodegradable group” is a group that can facilitate faster metabolism of a lipid in a subject. A biodegradable group can be, but is not limited to, —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group.

As used herein, an “aryl group” is a carbocyclic group including one or more aromatic rings. Examples of aryl groups include phenyl and naphthyl groups.

As used herein, a “heteroaryl group” is a heterocyclic group including one or more aromatic rings. Examples of heteroaryl groups include pyrrolyl, furyl, thiophenyl, imidazolyl, oxazolyl, and thiazolyl. Both aryl and heteroaryl groups can be optionally substituted. For example, M and M′ can be selected from the non-limiting group consisting of optionally substituted phenyl, oxazole, and thiazole. In the formulas herein, M and M′ can be independently selected from the list of biodegradable groups above.

Alkyl, alkenyl, and cyclyl (e.g., carbocyclyl and heterocyclyl) groups can be optionally substituted unless otherwise specified. Optional substituents can be selected from the group consisting of, but are not limited to, a halogen atom (e.g., a chloride, bromide, fluoride, or iodide group), a carboxylic acid (e.g., —C(O)OH), an alcohol (e.g., a hydroxyl, —OH), an ester (e.g., —C(O)OR or —OC(O)R), an aldehyde (e.g., —C(O)H), a carbonyl (e.g., —C(O)R, alternatively represented by C═O), an acyl halide (e.g., —C(O)X, in which X is a halide selected from bromide, fluoride, chloride, and iodide), a carbonate (e.g., —OC(O)OR), an alkoxy (e.g., —OR), an acetal (e.g., —C(OR)₂R″″, in which each OR are alkoxy groups that can be the same or different and R″″ is an alkyl or alkenyl group), a phosphate (e.g., P(O)₄₃), a thiol (e.g., —SH), a sulfoxide (e.g., —S(O)R), a sulfinic acid (e.g., —S(O)OH), a sulfonic acid (e.g., —S(O)₂OH), a thial (e.g., —C(S)H), a sulfate (e.g., S(O)₄²⁻), a sulfonyl (e.g., —S(O)₂—), an amide (e.g., —C(O)NR₂, or —N(R)C(O)R), an azido (e.g., —N₃), a nitro (e.g., —NO₂), a cyano (e.g., —CN), an isocyano (e.g., —NC), an acyloxy (e.g., —OC(O)R), an amino (e.g., —NR₂, —NRH, or —NH₂), a carbamoyl (e.g., —OC(O)NR₂, —OC(O)NRH, or —OC(O)NH₂), a sulfonamide (e.g., —S(O)₂NR₂, —S(O)₂NRH, —S(O)₂NH₂, —N(R)S(O)₂R, —N(H)S(O)₂R, —N(R)S(O)₂H, or —N(H)S(O)₂H), an alkyl group, an alkenyl group, and a cyclyl (e.g., carbocyclyl or heterocyclyl) group.

In any of the preceding, R is an alkyl or alkenyl group, as defined herein. In some embodiments, the substituent groups themselves can be further substituted with, for example, one, two, three, four, five, or six substituents as defined herein. For example, a C₁₋₆ alkyl group can be further substituted with one, two, three, four, five, or six substituents as described herein.

The compounds of any one of formulae (I), (IA), (II), (IIa), (IIb), (IIc), (IId), and (IIe) include one or more of the following features when applicable.

In some embodiments, R₄ is selected from the group consisting of a C₃₋₆ carbocycle, —(CH₂)ₙQ, —(CH₂)ₙCHQR, —CHQR, and —CQ(R)₂, where Q is selected from a C₃₋₆ carbocycle, 5- to 14-membered aromatic or non-aromatic heterocycle having one or more heteroatoms selected from N, O, S, and P, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, and —C(R)N(R)₂C(O)OR, and each n is independently selected from 1, 2, 3, 4, and 5.

In another embodiment, R₄ is selected from the group consisting of a C₃₋₆ carbocycle, —(CH₂)ₙQ, —(CH₂)ₙCHQR, —CHQR, and —CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —C(R)N(R)₂C(O)OR, and a 5- to 14-membered heterocycloalkyl having one or more heteroatoms selected from N, O, and S which is substituted with one or more substituents selected from oxo (═O), OH, amino, and C₁₋₃ alkyl, and each n is independently selected from 1, 2, 3, 4, and 5.

In another embodiment, R₄ is selected from the group consisting of a C₃₋₆ carbocycle, —(CH₂)ₙQ, —(CH₂)ₙCHQR, —CHQR, and —CQ(R)₂, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —C(R)N(R)₂C(O)OR, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl.

In another embodiment, R₄ is selected from the group consisting of a C₃₋₆ carbocycle, —(CH₂)ₙQ, —(CH₂)ₙCHQR, —CHQR, and —CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —C(R)N(R)₂C(O)OR, and each n is independently selected from 1, 2, 3, 4, and 5.

In another embodiment, R₄ is unsubstituted C₁₄ alkyl, e.g., unsubstituted methyl.

In certain embodiments, the disclosure provides a compound having the Formula (I), wherein R₄ is —(CH₂)ₙQ or —(CH₂)ₙCHQR, where Q is —N(R)₂, and n is selected from 3, 4, and 5.

In certain embodiments, the disclosure provides a compound having the Formula (I), wherein R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5.

In certain embodiments, the disclosure provides a compound having the Formula (I), wherein R₂ and R₃ are independently selected from the group consisting of C₂₋₁₄ alkyl, C₂₋₁₄ alkenyl, —R*YR″, —YR″, and —R*OR″, or R₂ and R₃, together with the atom to which they are attached, form a heterocycle or carbocycle, and R₄ is —(CH₂)ₙQ or —(CH₂)ₙCHQR, where Q is —N(R)₂, and n is selected from 3, 4, and 5.

In certain embodiments, R₂ and R₃ are independently selected from the group consisting of C₂₋₁₄ alkyl, C₂₋₁₄ alkenyl, —R*YR″, —YR″, and —R*OR″, or R₂ and R₃, together with the atom to which they are attached, form a heterocycle or carbocycle.

In some embodiments, R₁ is selected from the group consisting of C₅₋₂₀ alkyl and C₅₋₂₀ alkenyl.

In other embodiments, R₁ is selected from the group consisting of —R*YR″, —YR″, and —R″M′R′.

In certain embodiments, R₁ is selected from —R*YR″ and —YR″. In some embodiments, Y is a cyclopropyl group. In some embodiments, R* is C₈ alkyl or C₈ alkenyl. In certain embodiments, R″ is C₃₋₁₂ alkyl. For example, R″ can be C₃ alkyl. For example, R″ can be C₄₋₈ alkyl (e.g., C₄, C₅, C₆, C₇, or C₈ alkyl).

In some embodiments, R₁ is C₅₋₂₀ alkyl. In some embodiments, R₁ is C₆ alkyl. In some embodiments, R₁ is C₈ alkyl. In other embodiments, R₁ is C₉ alkyl. In certain embodiments, R₁ is C₁₄ alkyl. In other embodiments, R₁ is C₁₋₈ alkyl.

In some embodiments, R₁ is C₅₋₂₀ alkenyl. In certain embodiments, R₁ is C₁₈ alkenyl. In some embodiments, R₁ is linoleyl.

In certain embodiments, R₁ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl, or heptadeca-9-yl). In certain embodiments, R₁ is

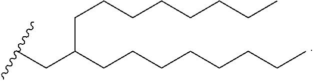 [SMILES: *CC(CCCCCCCC)CCCCCCCC]

In certain embodiments, R₁ is unsubstituted C₅₋₂₀ alkyl or C₅₋₂₀ alkenyl. In certain embodiments, R′ is substituted C₅₋₂₀ alkyl or C₅₋₂₀ alkenyl (e.g., substituted with a C₃₋₆ carbocycle such as 1-cyclopropylnonyl).

In other embodiments, R₁ is —R″M′R′.

In some embodiments, R′ is selected from —R*YR″ and —YR″. In some embodiments, Y is C₃₋₈ cycloalkyl. In some embodiments, Y is C₆₋₁₀ aryl. In some embodiments, Y is a cyclopropyl group. In some embodiments, Y is a cyclohexyl group. In certain embodiments, R* is C₁ alkyl.

In some embodiments, R″ is selected from the group consisting of C₃₋₁₂ alkyl and C₃₋₁₂ alkenyl. In some embodiments, R″ adjacent to Y is C₁ alkyl. In some embodiments, R″ adjacent to Y is C₄₋₉ alkyl (e.g., C₄, C₅, C₆, C₇ or C₈ or C₉ alkyl).

In some embodiments, R′ is selected from C₄ alkyl and C₄ alkenyl. In certain embodiments, R′ is selected from C₅ alkyl and C₅ alkenyl. In some embodiments, R′ is selected from C₆ alkyl and C₆ alkenyl. In some embodiments, R′ is selected from C₇ alkyl and C₇ alkenyl. In some embodiments, R′ is selected from C₉ alkyl and C₉ alkenyl.

In other embodiments, R′ is selected from C₁₁ alkyl and C₁₁ alkenyl. In other embodiments, R′ is selected from C₁₂ alkyl, C₁₂ alkenyl, C₁₃ alkyl, C₁₃ alkenyl, C₁₄ alkyl, C₁₄ alkenyl, C₁₅ alkyl, C₁₅ alkenyl, C₁₆ alkyl, C₁₆ alkenyl, C₁₇ alkyl, C₁₇ alkenyl, C₁₈ alkyl, and C₁₈ alkenyl. In certain embodiments, R′ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl or heptadeca-9-yl). In certain embodiments, R′ is

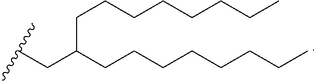 [SMILES: *CC(CCCCCCCC)CCCCCCCC]

In certain embodiments, R′ is unsubstituted C₁₋₁₈ alkyl. In certain embodiments, R′ is substituted C₁₋₁₈ alkyl (e.g., C₁₋₁₅ alkyl substituted with a C₃₋₆ carbocycle such as 1-cyclopropylnonyl).

In some embodiments, R″ is selected from the group consisting of C₃₋₁₄ alkyl and C₃₋₁₄ alkenyl. In some embodiments, R″ is C₃ alkyl, C₄ alkyl, C₅ alkyl, C₆ alkyl, C₇ alkyl, or C₈ alkyl. In some embodiments, R″ is C₉ alkyl, C₁₀ alkyl, C₁₁ alkyl, C₁₂ alkyl, C₁₃ alkyl, or C₁₄ alkyl.

In some embodiments, M′ is —C(O)O—. In some embodiments, M′ is —OC(O)—.

In other embodiments, M′ is an aryl group or heteroaryl group. For example, M′ can be selected from the group consisting of phenyl, oxazole, and thiazole.

In some embodiments, M is —C(O)O—In some embodiments, M is —OC(O)—. In some embodiments, M is —C(O)N(R′)—. In some embodiments, M is —P(O)(OR′)O—.

In other embodiments, M is an aryl group or heteroaryl group. For example, M can be selected from the group consisting of phenyl, oxazole, and thiazole.

In some embodiments, M is the same as M′. In other embodiments, M is different from M′.

In some embodiments, each R₅ is H. In certain such embodiments, each R₆ is also H.

In some embodiments, R₇ is H. In other embodiments, R₇ is C₁₋₃ alkyl (e.g., methyl, ethyl, propyl, or i-propyl).

In some embodiments, R₂ and R₃ are independently C₅₋₁₄ alkyl or C₅₋₁₄ alkenyl.

In some embodiments, R₂ and R₃ are the same. In some embodiments, R₂ and R₃ are C₈ alkyl. In certain embodiments, R₂ and R₃ are C₂ alkyl. In other embodiments, R₂ and R₃ are C₃ alkyl. In some embodiments, R₂ and R₃ are C₄ alkyl. In certain embodiments, R₂ and R₃ are C₅ alkyl. In other embodiments, R₂ and R₃ are C₆ alkyl. In some embodiments, R₂ and R₃ are C₇ alkyl.

In other embodiments, R₂ and R₃ are different. In certain embodiments, R₂ is C₈ alkyl.

In some embodiments, R₃ is C₁₋₇(e.g., C₁, C₂, C₃, C₄, C₅, C₆, or C₇ alkyl) or C₉ alkyl.

In some embodiments, R₇ and R₃ are H.

In certain embodiments, R₂ is H.

In some embodiments, m is 5, 7, or 9.

In some embodiments, R₄ is selected from —(CH₂)ₙQ and —(CH₂)ₙCHQR.

In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), —C(R)N(R)₂C(O)OR, a carbocycle, and a heterocycle.

In certain embodiments, Q is —OH.

In certain embodiments, Q is a substituted or unsubstituted 5- to 10-membered heteroaryl, e.g., Q is an imidazole, a pyrimidine, a purine, 2-amino-1,9-dihydro-6H-purin-6-one-9-yl (or guanin-9-yl), adenin-9-yl, cytosin-1-yl, or uracil-1-yl. In certain embodiments, Q is a substituted 5- to 14-membered heterocycloalkyl, e.g., substituted with one or more substituents selected from oxo (═O), OH, amino, and C₁₋₃ alkyl. For example, Q is 4-methylpiperazinyl, 4-(4-methoxybenzyl)piperazinyl, or isoindolin-2-yl-1,3-dione.

In certain embodiments, Q is an unsubstituted or substituted C₆₋₁₀ aryl (such as phenyl) or C₃₋₆ cycloalkyl.

In some embodiments, n is 1. In other embodiments, n is 2. In further embodiments, n is 3. In certain other embodiments, n is 4. For example, R₄ can be —(CH₂)₂OH. For example, R₄ can be —(CH₂)₃OH. For example, R₄ can be —(CH₂)₄OH. For example, R₄ can be benzyl. For example, R₄ can be 4-methoxybenzyl.

In some embodiments, R₄ is a C₃₋₆ carbocycle. In some embodiments, R₄ is a C₃₋₆ cycloalkyl. For example, R₄ can be cyclohexyl optionally substituted with e.g., OH, halo, C₁₋₆ alkyl, etc. For example, R₄ can be 2-hydroxycyclohexyl.

In some embodiments, R is H.

In some embodiments, R is unsubstituted C₁₋₃ alkyl or unsubstituted C₂₋₃ alkenyl. For example, R₄ can be —CH₂CH(OH)CH₃ or —CH₂CH(OH)CH₂CH₃.

In some embodiments, R is substituted C₁₋₃ alkyl, e.g., CH₂OH. For example, R₄ can be —CH₂CH(OH)CH₂OH.

In some embodiments, R₂ and R₃, together with the atom to which they are attached, form a heterocycle or carbocycle. In some embodiments, R₂ and R₃, together with the atom to which they are attached, form a 5- to 14-membered aromatic or non-aromatic heterocycle having one or more heteroatoms selected from N, O, S, and P. In some embodiments, R₂ and R₃, together with the atom to which they are attached, form an optionally substituted C₃₋₂₀ carbocycle (e.g., C₃₋₁₈ carbocycle, C₃₋₁₅ carbocycle, C₃₋₁₂ carbocycle, or C₃₋₁₀ carbocycle), either aromatic or non-aromatic. In some embodiments, R₂ and R₃, together with the atom to which they are attached, form a C₃₋₆ carbocycle. In other embodiments, R₂ and R₃, together with the atom to which they are attached, form a C₆ carbocycle, such as a cyclohexyl or phenyl group. In certain embodiments, the heterocycle or C₃₋₆ carbocycle is substituted with one or more alkyl groups (e.g., at the same ring atom or at adjacent or non-adjacent ring atoms). For example, R₂ and R₃, together with the atom to which they are attached, can form a cyclohexyl or phenyl group bearing one or more C₅ alkyl substitutions. In certain embodiments, the heterocycle or C₃₋₆ carbocycle formed by R₂ and R₃, is substituted with a carbocycle groups. For example, R₂ and R₃, together with the atom to which they are attached, can form a cyclohexyl or phenyl group that is substituted with cyclohexyl. In some embodiments, R₂ and R₃, together with the atom to which they are attached, form a C₇₋₁₅ carbocycle, such as a cycloheptyl, cyclopentadecanyl, or naphthyl group.

In some embodiments, R₄ is selected from —(CH₂)ₙQ and —(CH₂)ₙCHQR. In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle. In other embodiments, Q is selected from the group consisting of an imidazole, a pyrimidine, and a purine.

In some embodiments, R₂ and R₃, together with the atom to which they are attached, form a heterocycle or carbocycle. In some embodiments, R₂ and R₃, together with the atom to which they are attached, form a C₃₋₆ carbocycle, such as a phenyl group. In certain embodiments, the heterocycle or C₃₋₆ carbocycle is substituted with one or more alkyl groups (e.g., at the same ring atom or at adjacent or non-adjacent ring atoms). For example, R₂ and R₃, together with the atom to which they are attached, can form a phenyl group bearing one or more C₅ alkyl substitutions.

In some embodiments, a subset of compounds of Formula (I) includes those of Formula (IIa), (IIb), (IIc), or (IIe):

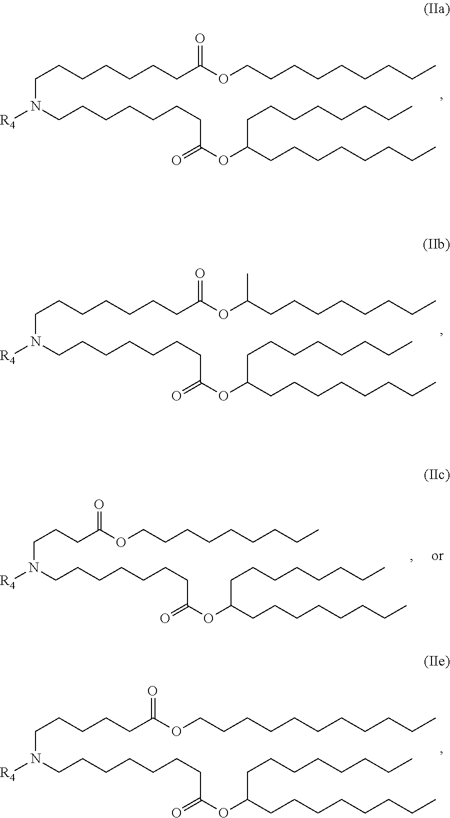

or a salt or isomer thereof, wherein R₄ is as described herein.

In some embodiments, a subset of compounds of Formula (I) includes those of Formula (IId):

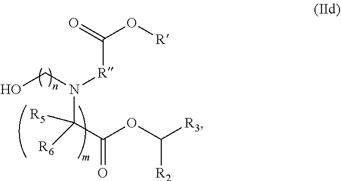

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, the pharmaceutical compositions of the present disclosure, the compound of formula (I) is selected from the group consisting of:

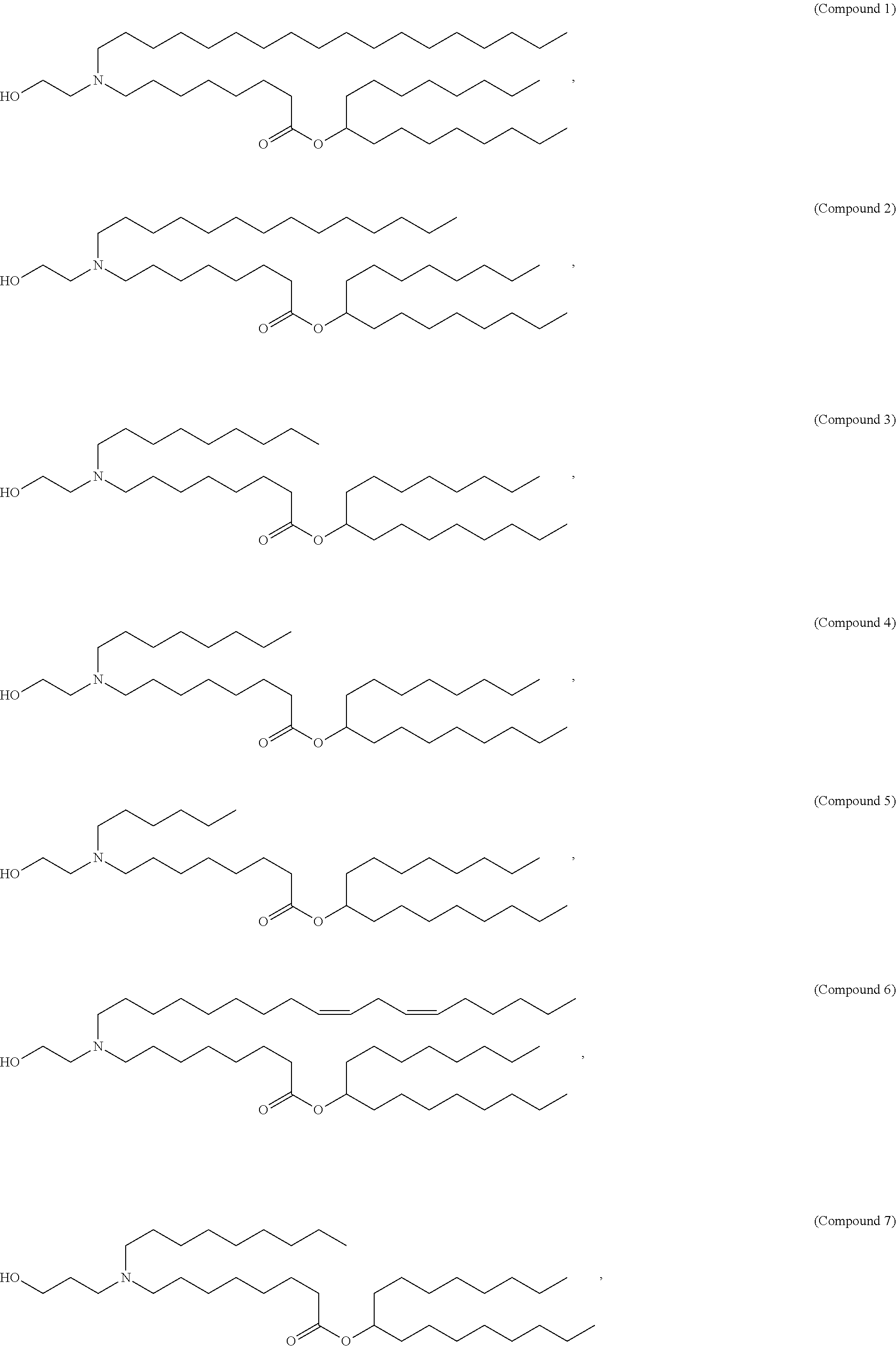

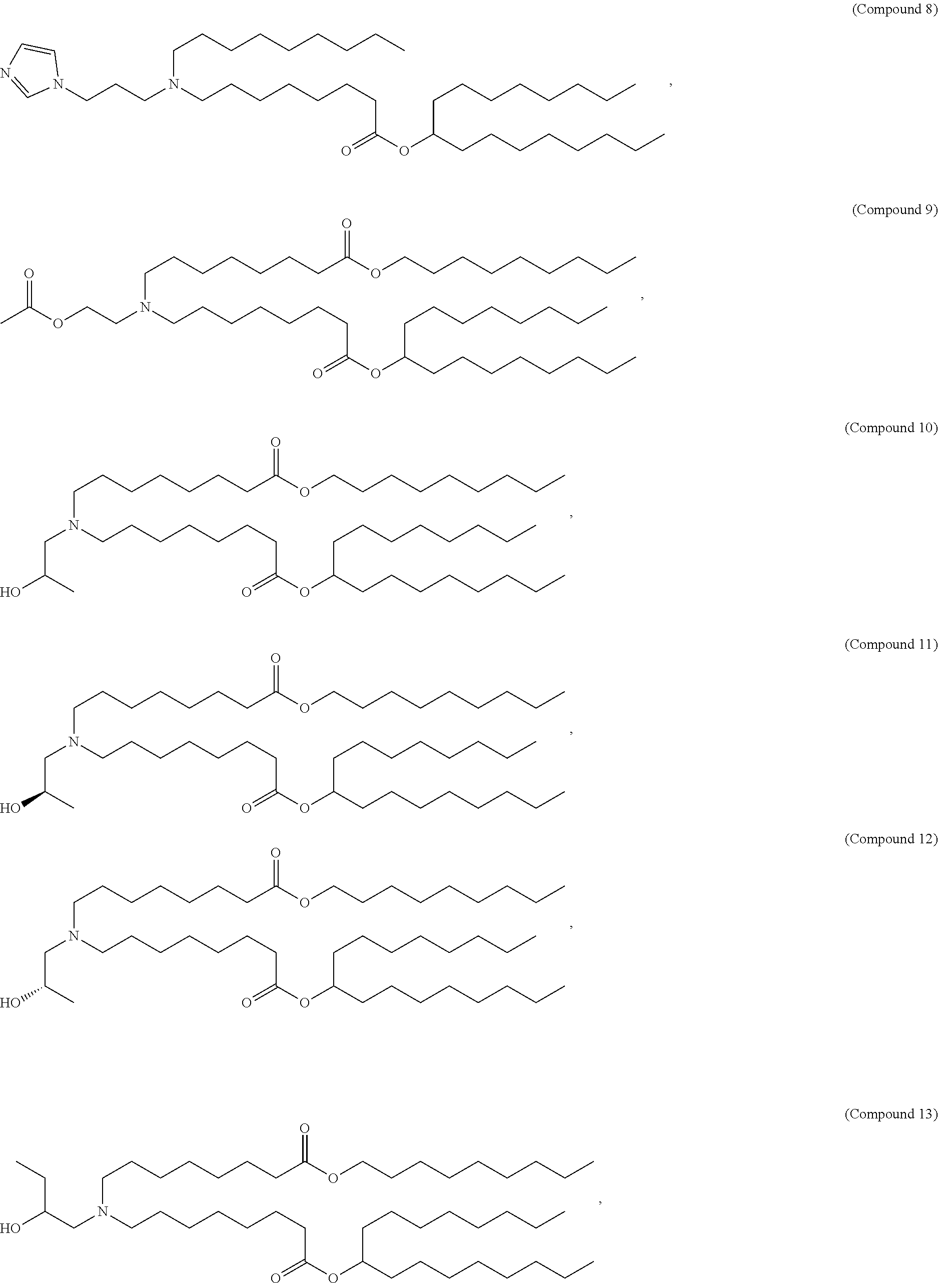

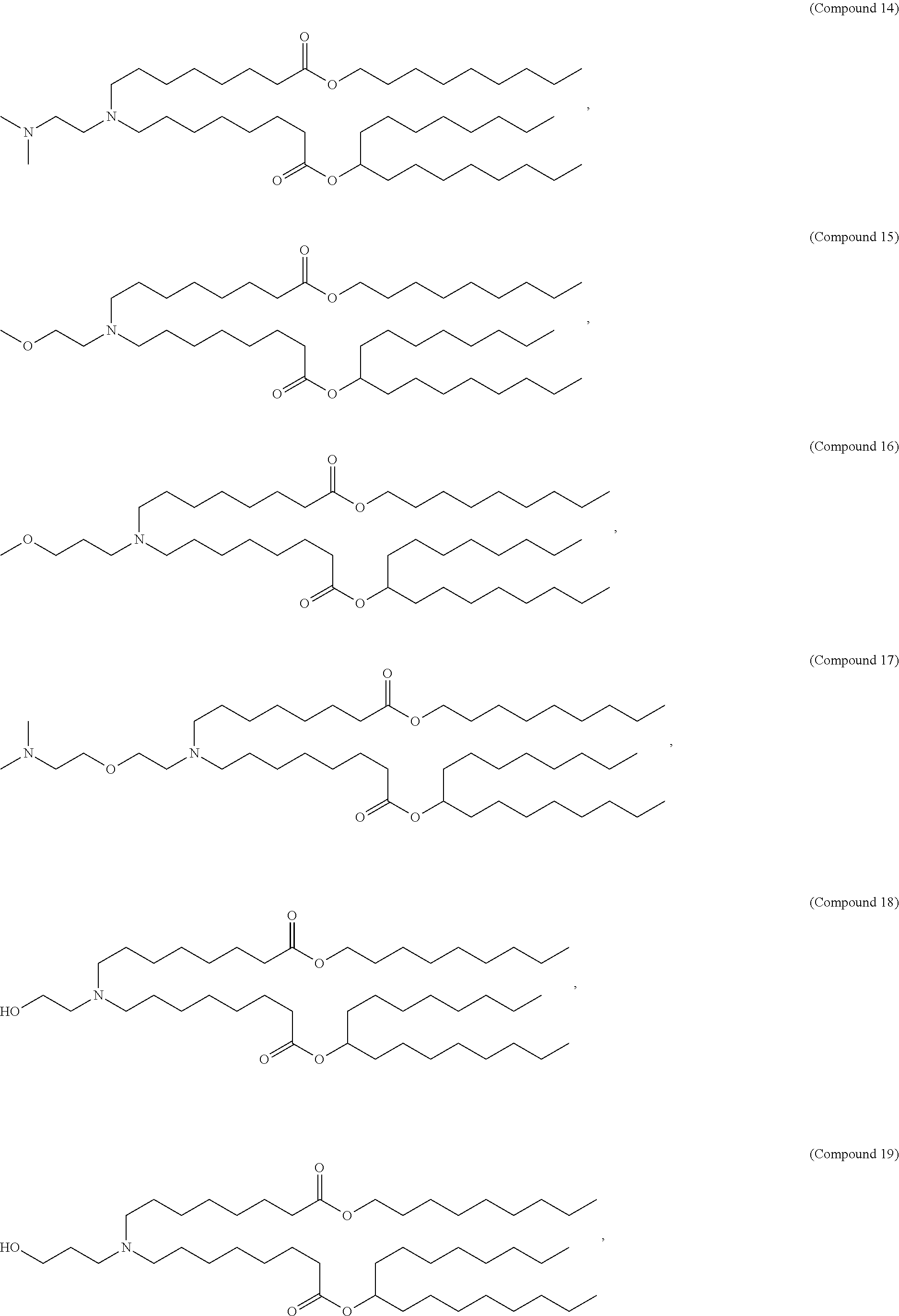

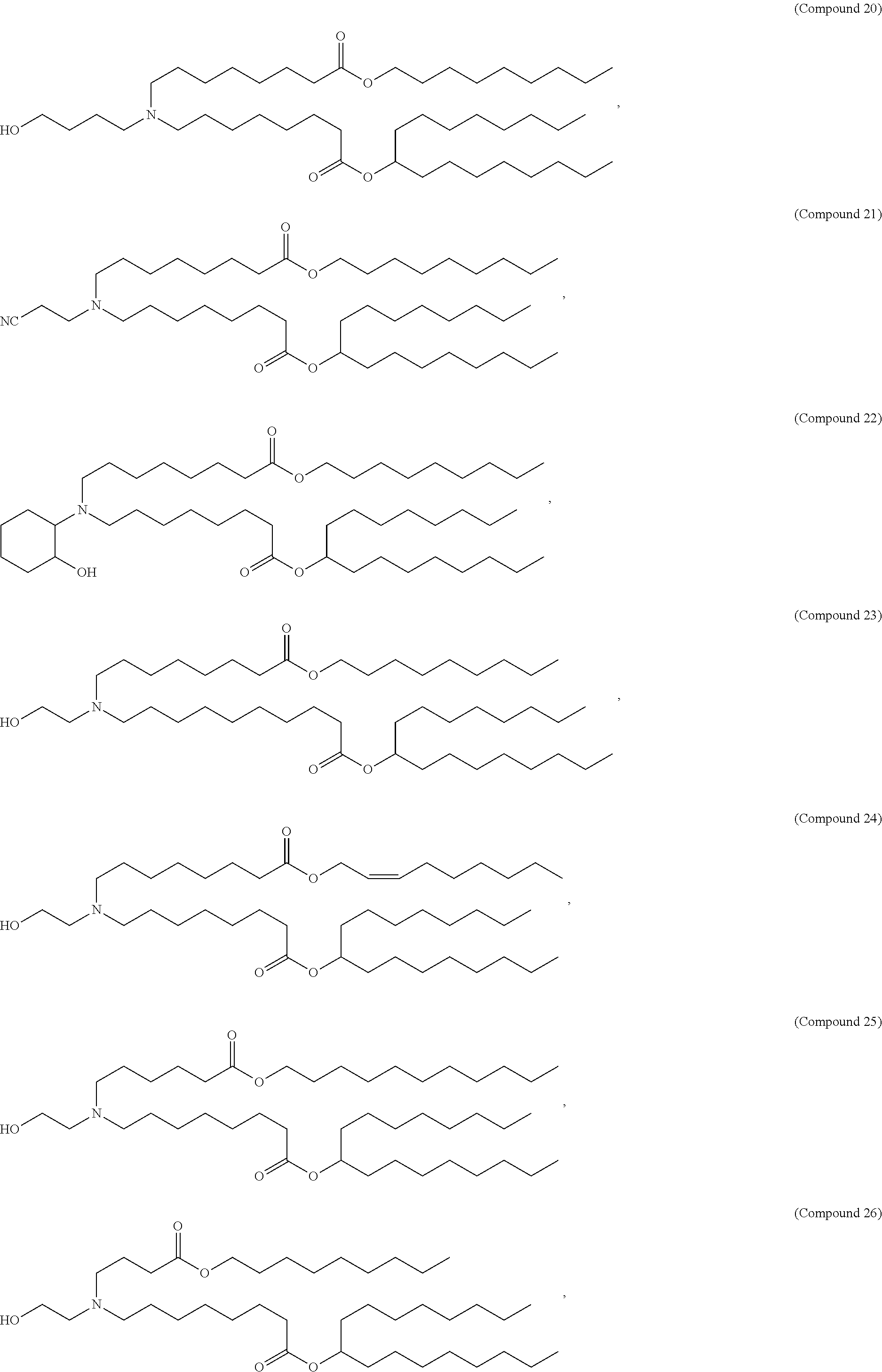

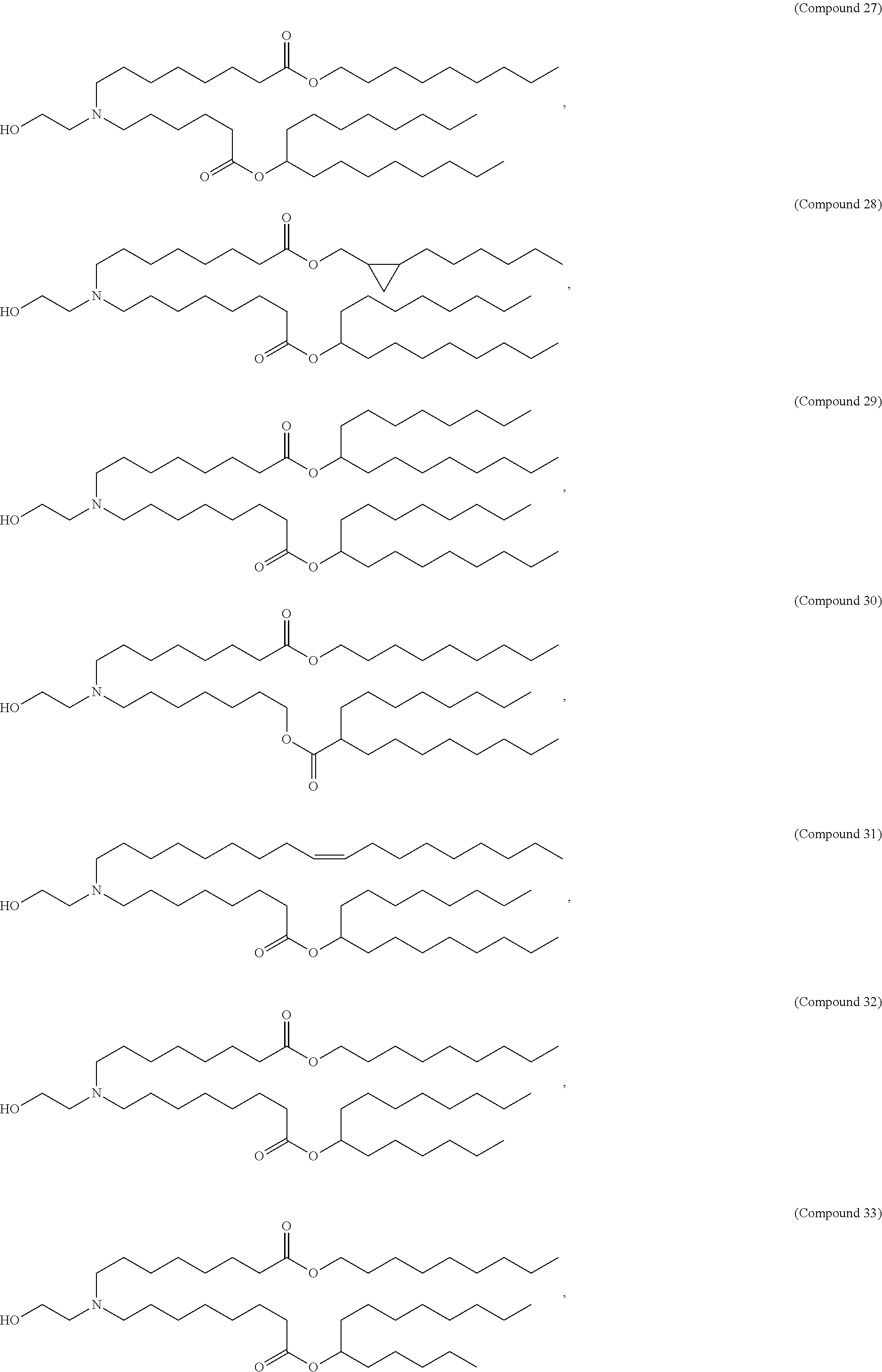

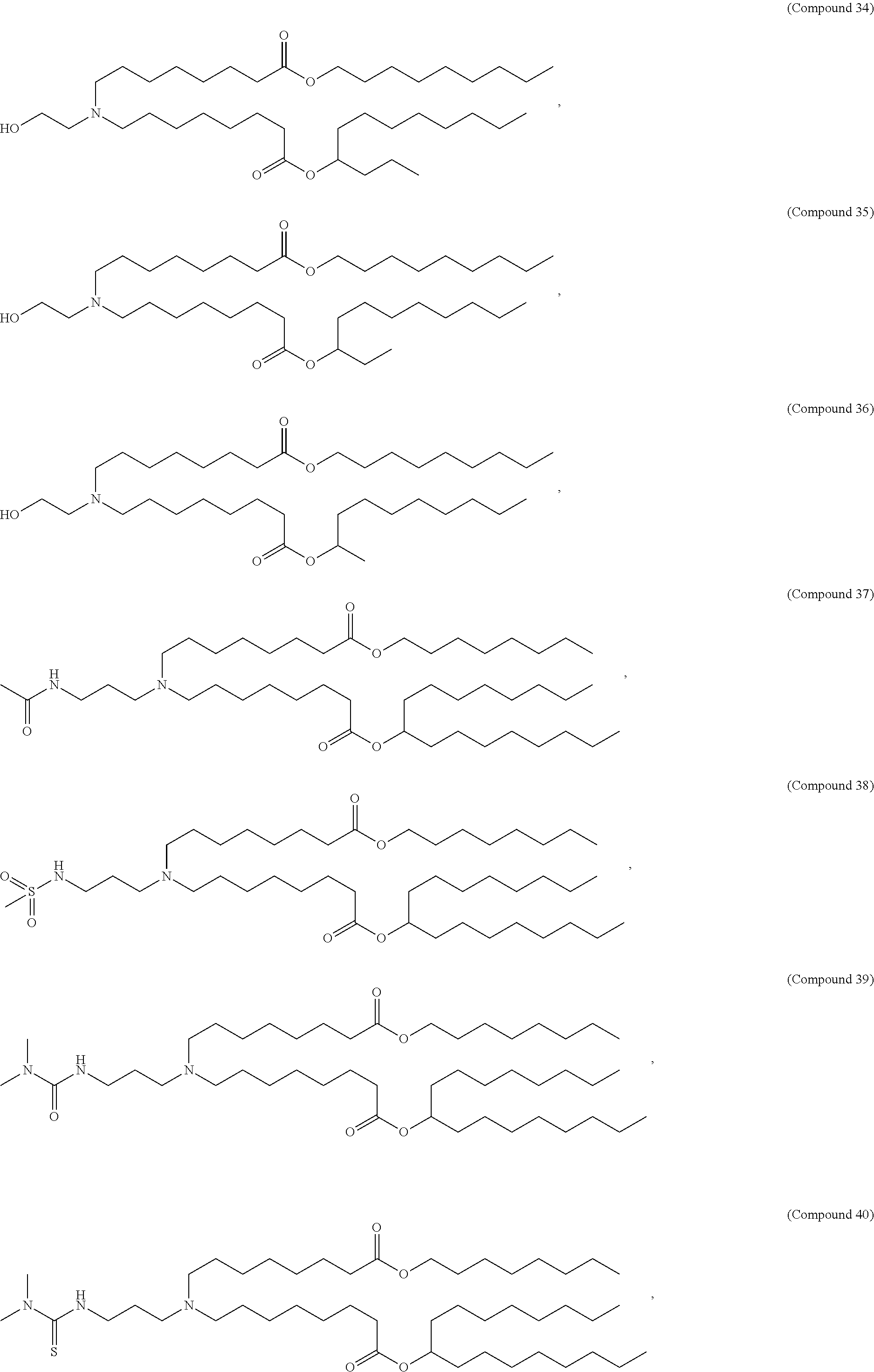

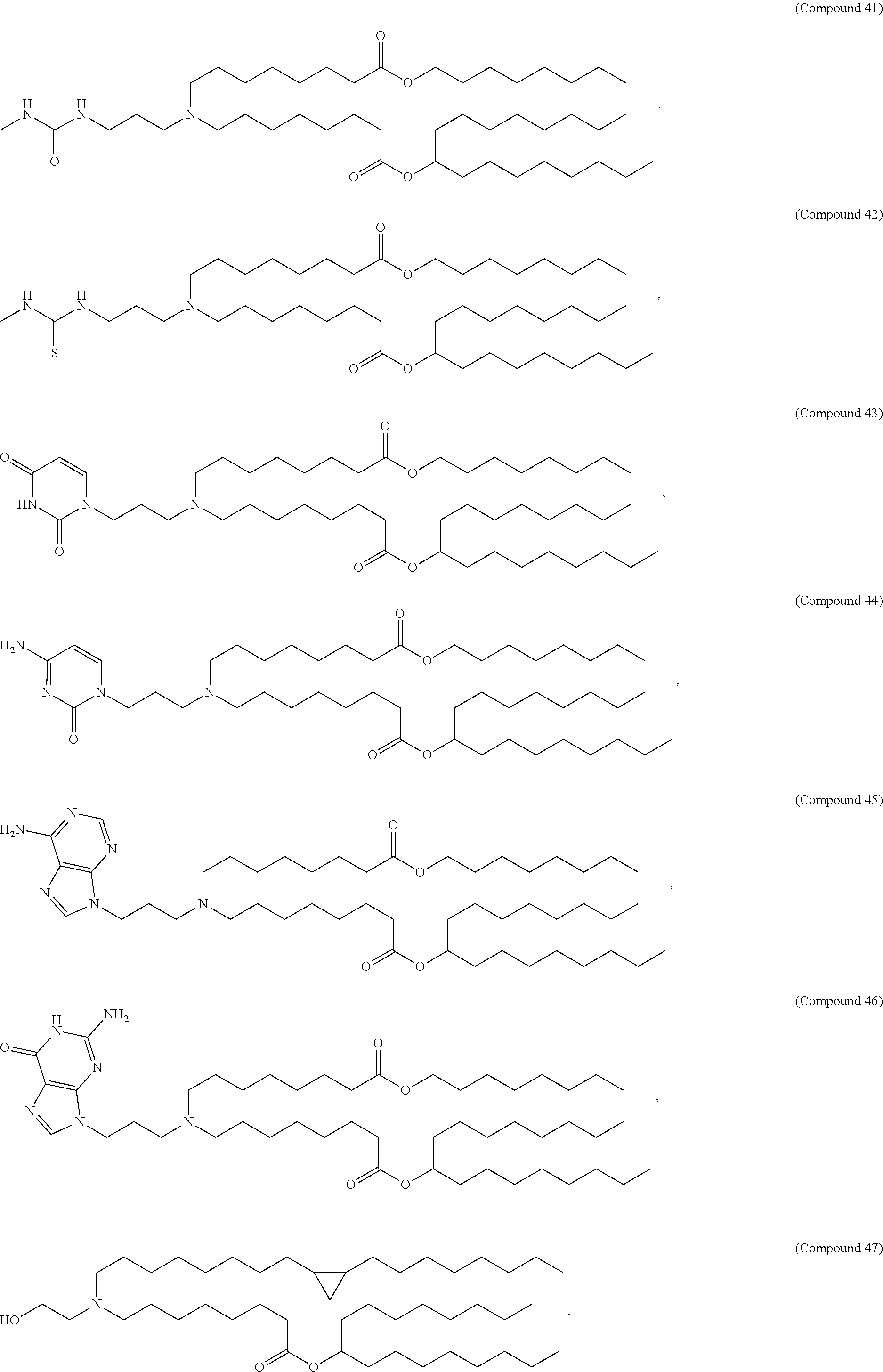

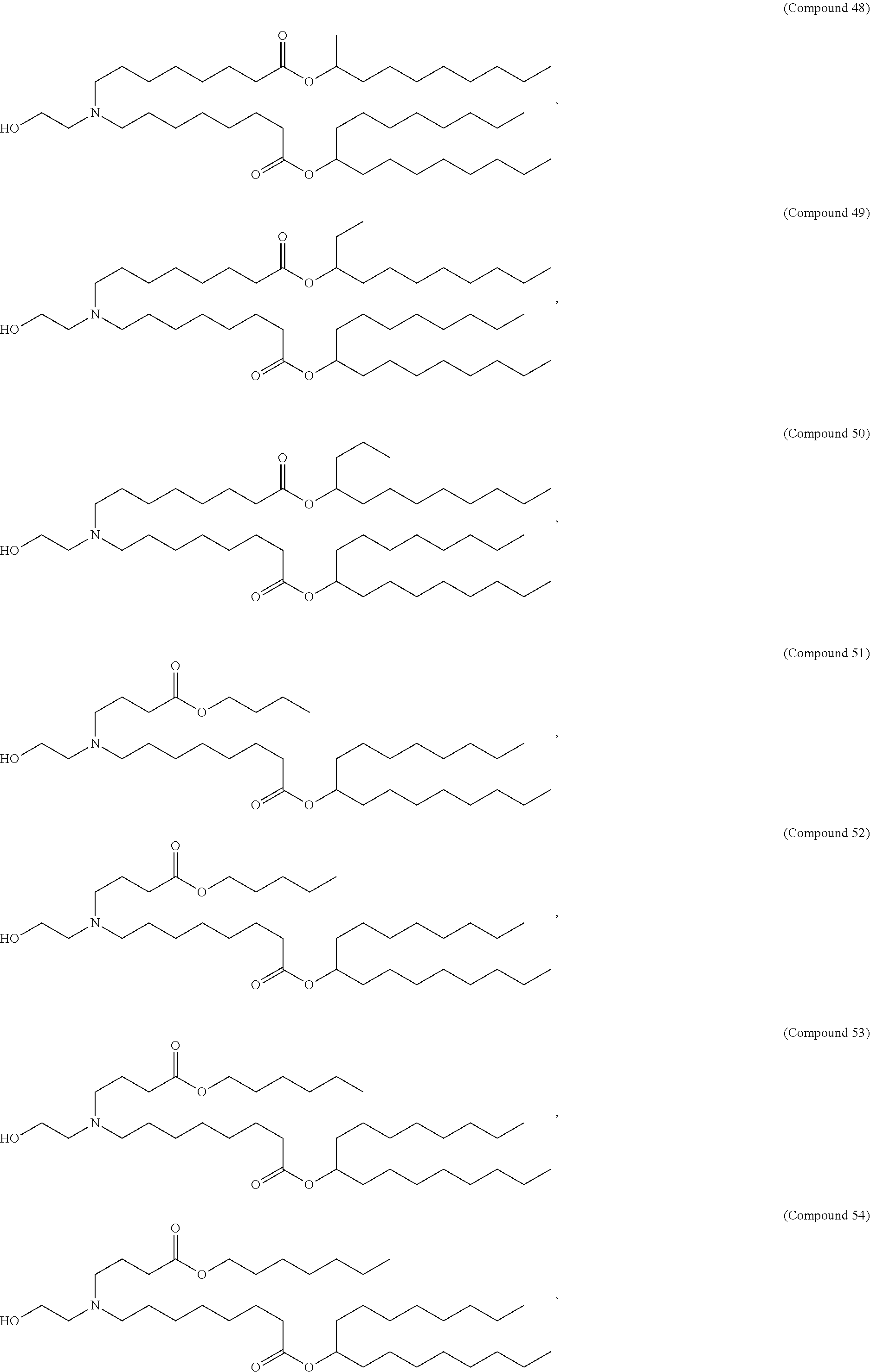

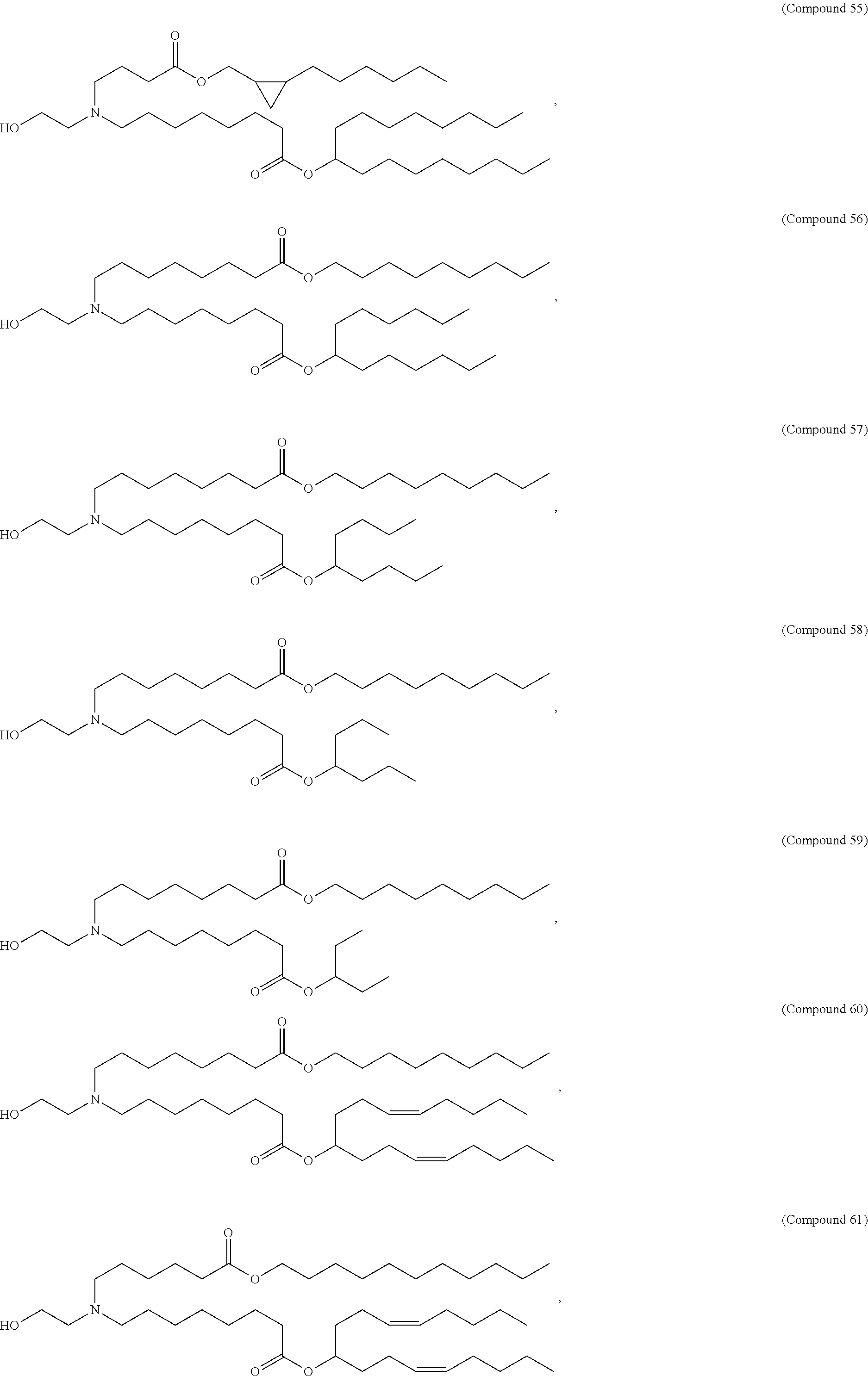

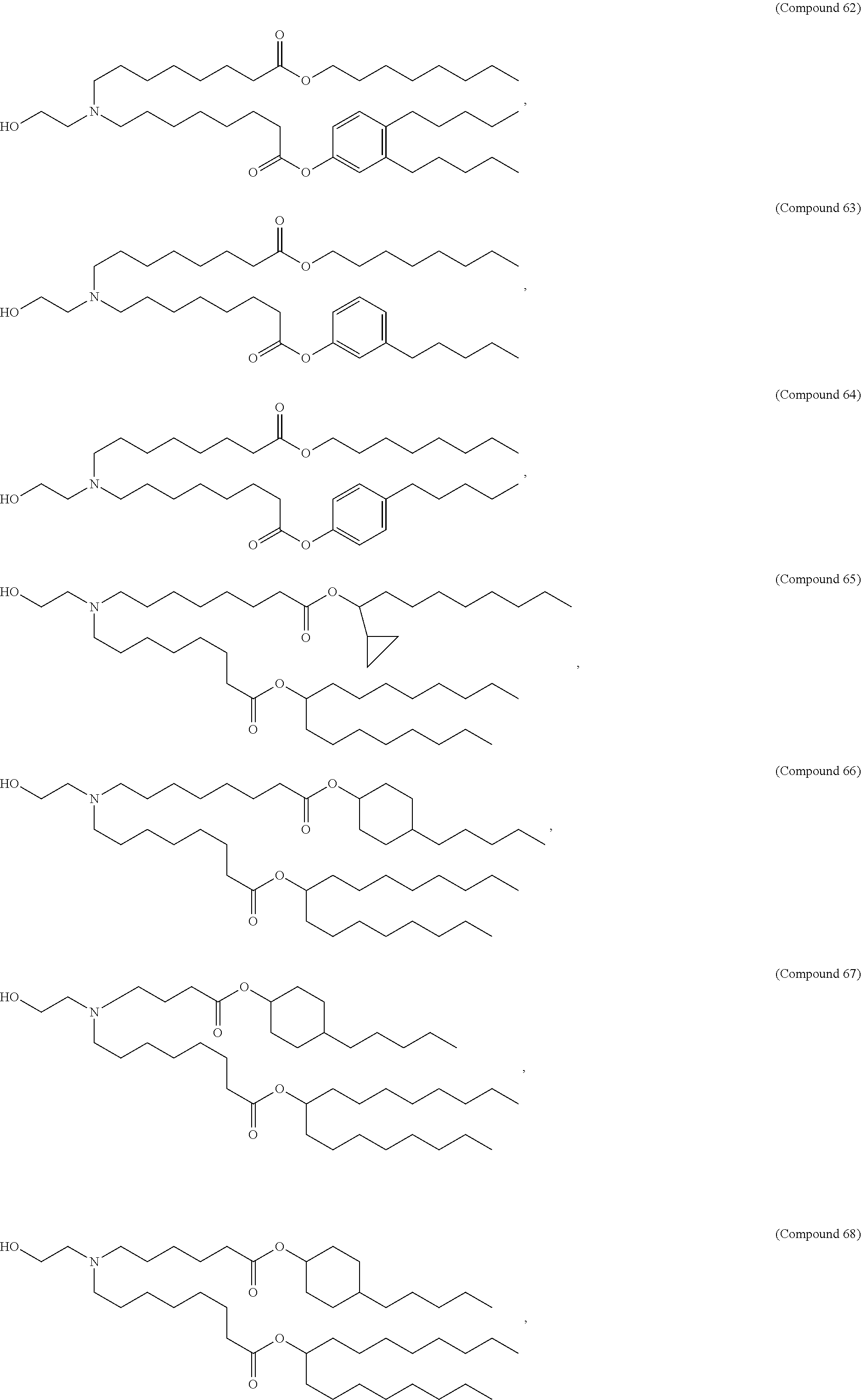

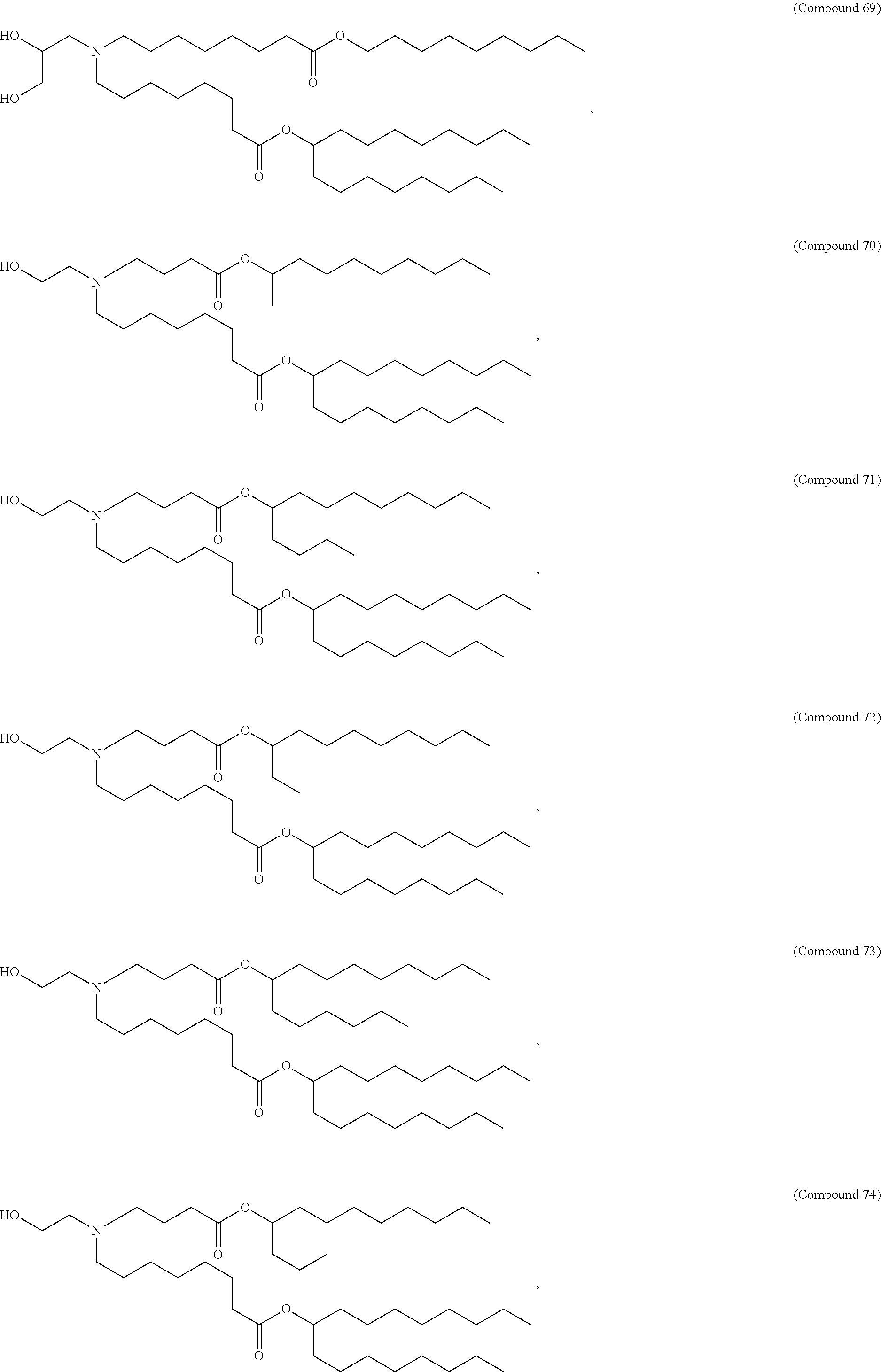

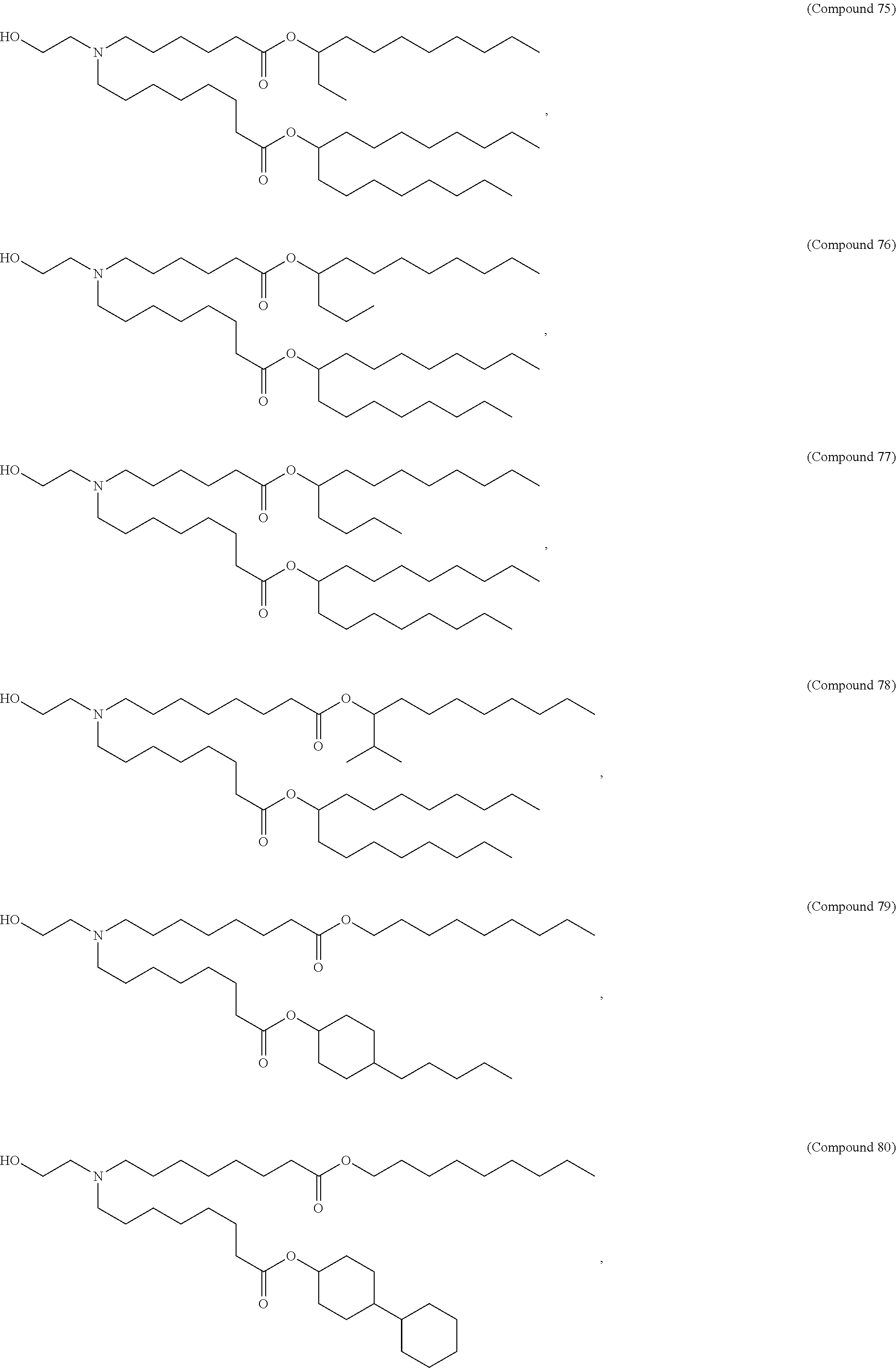

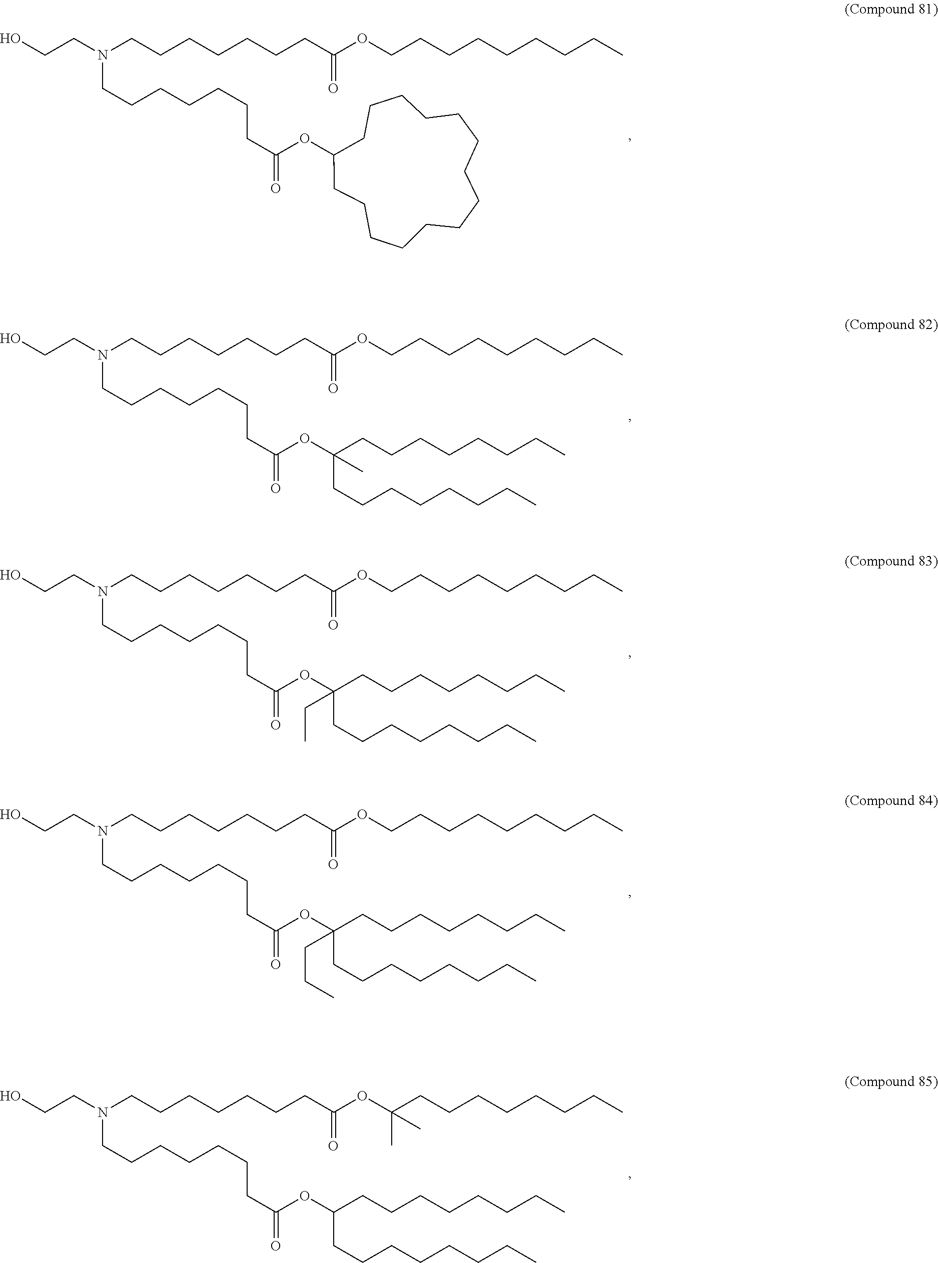

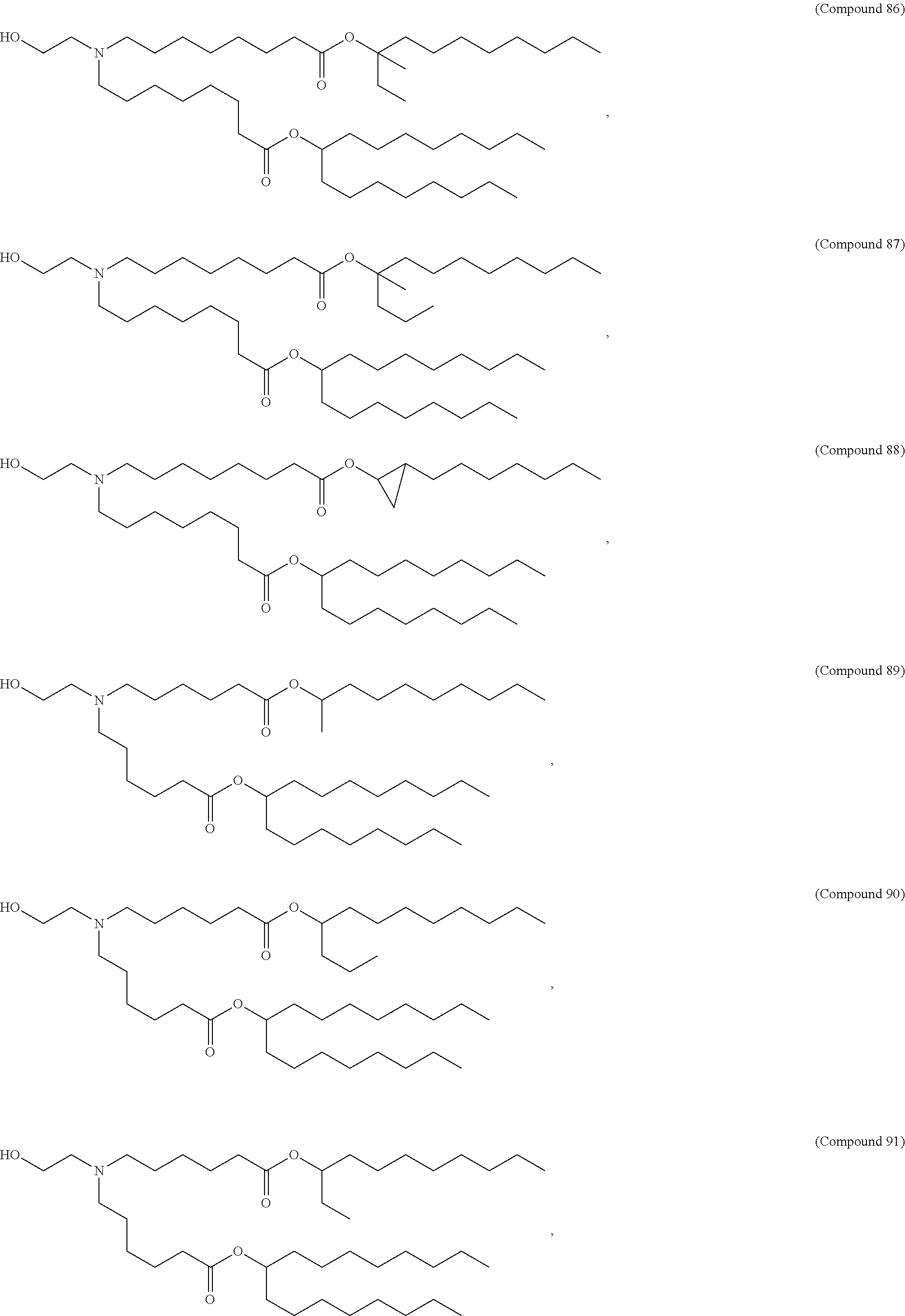

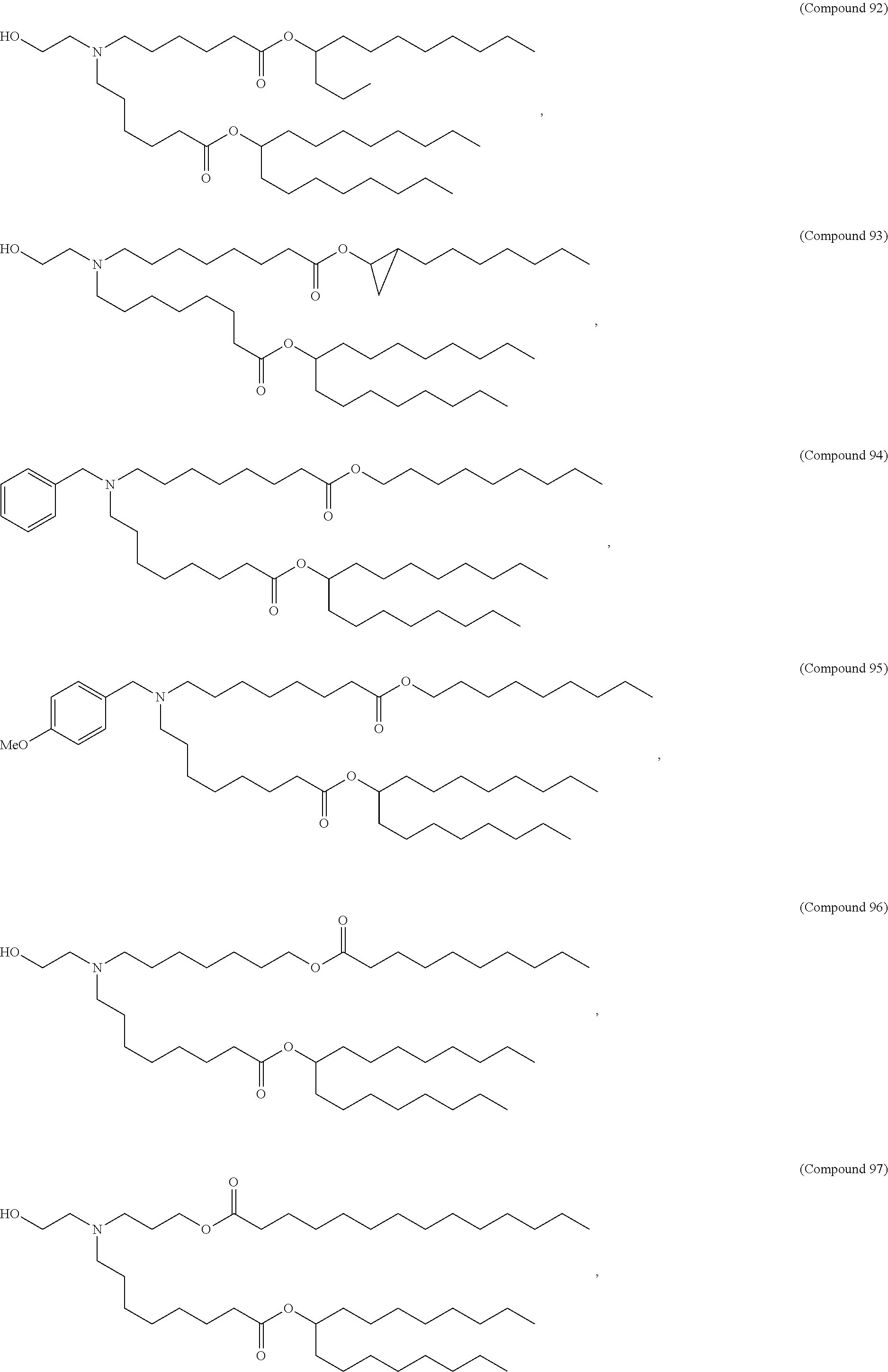

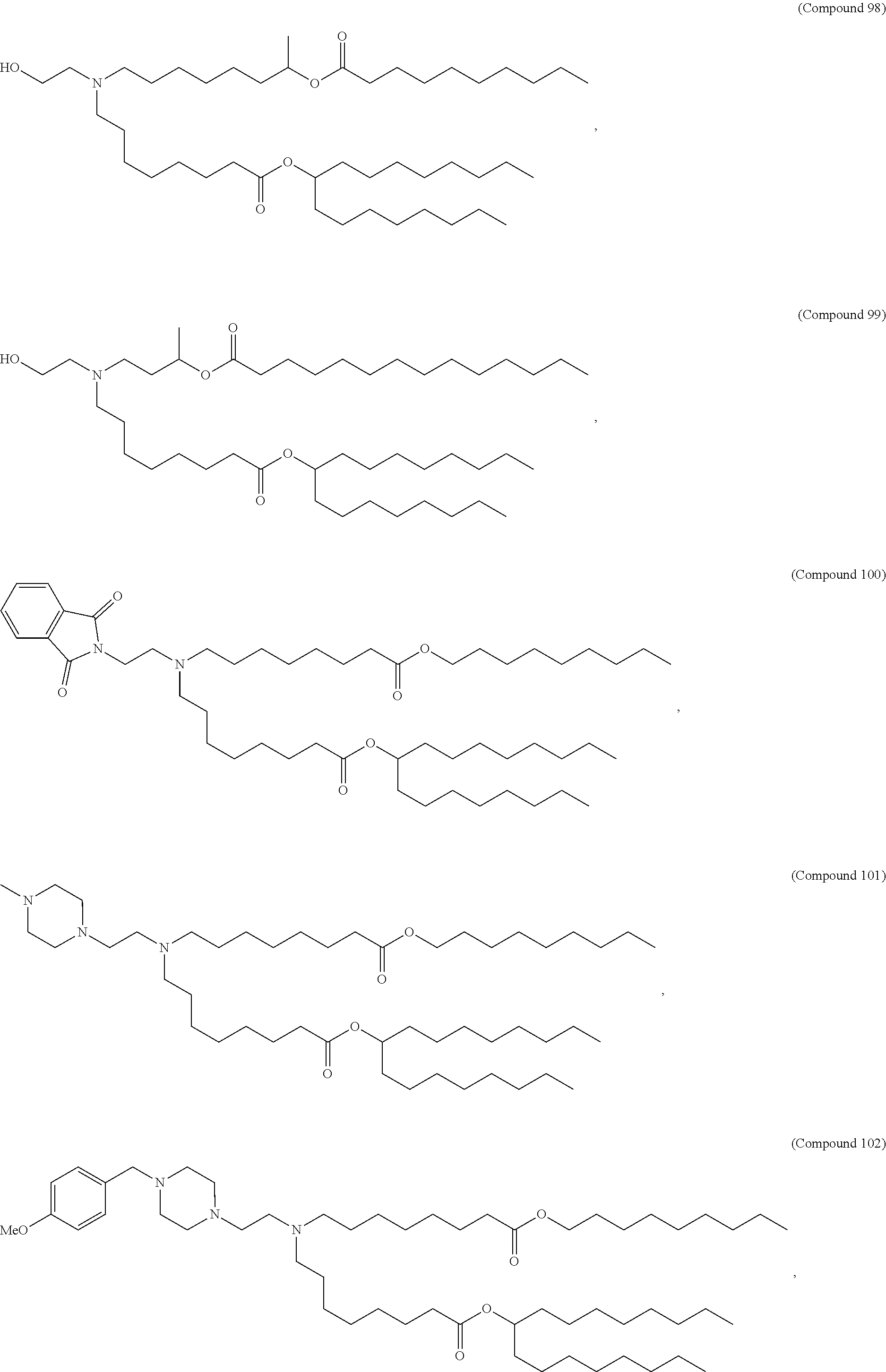

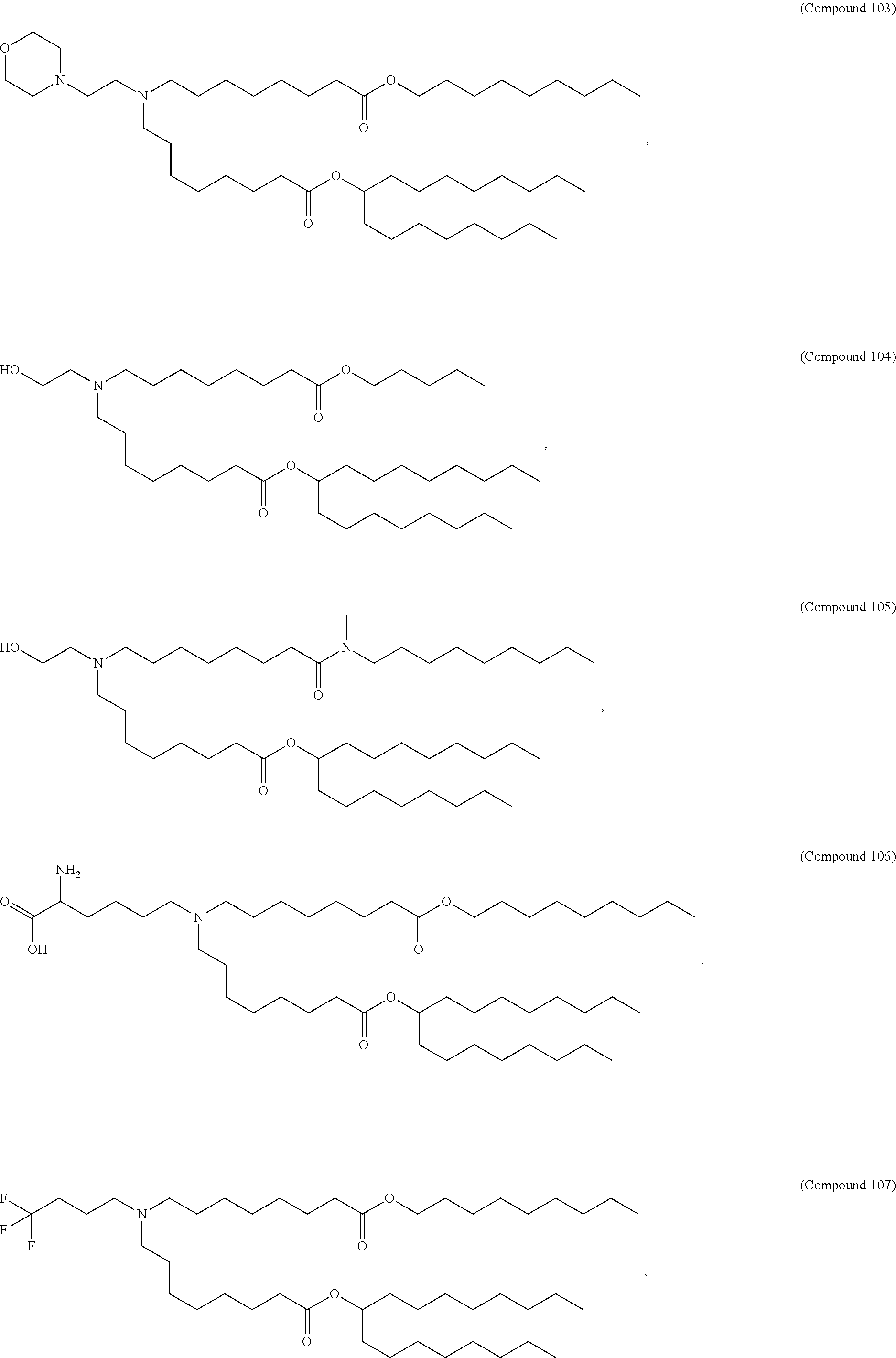

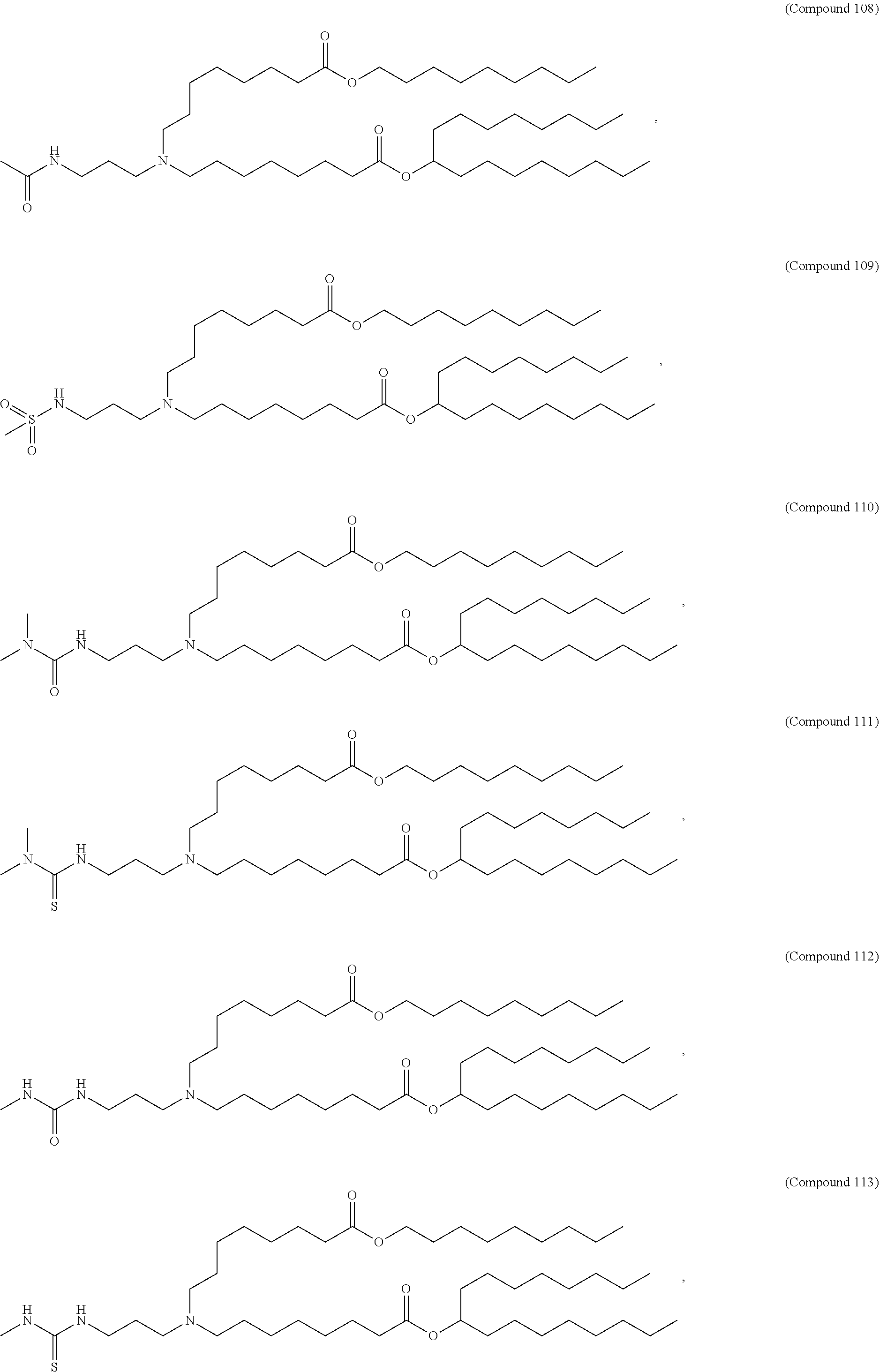

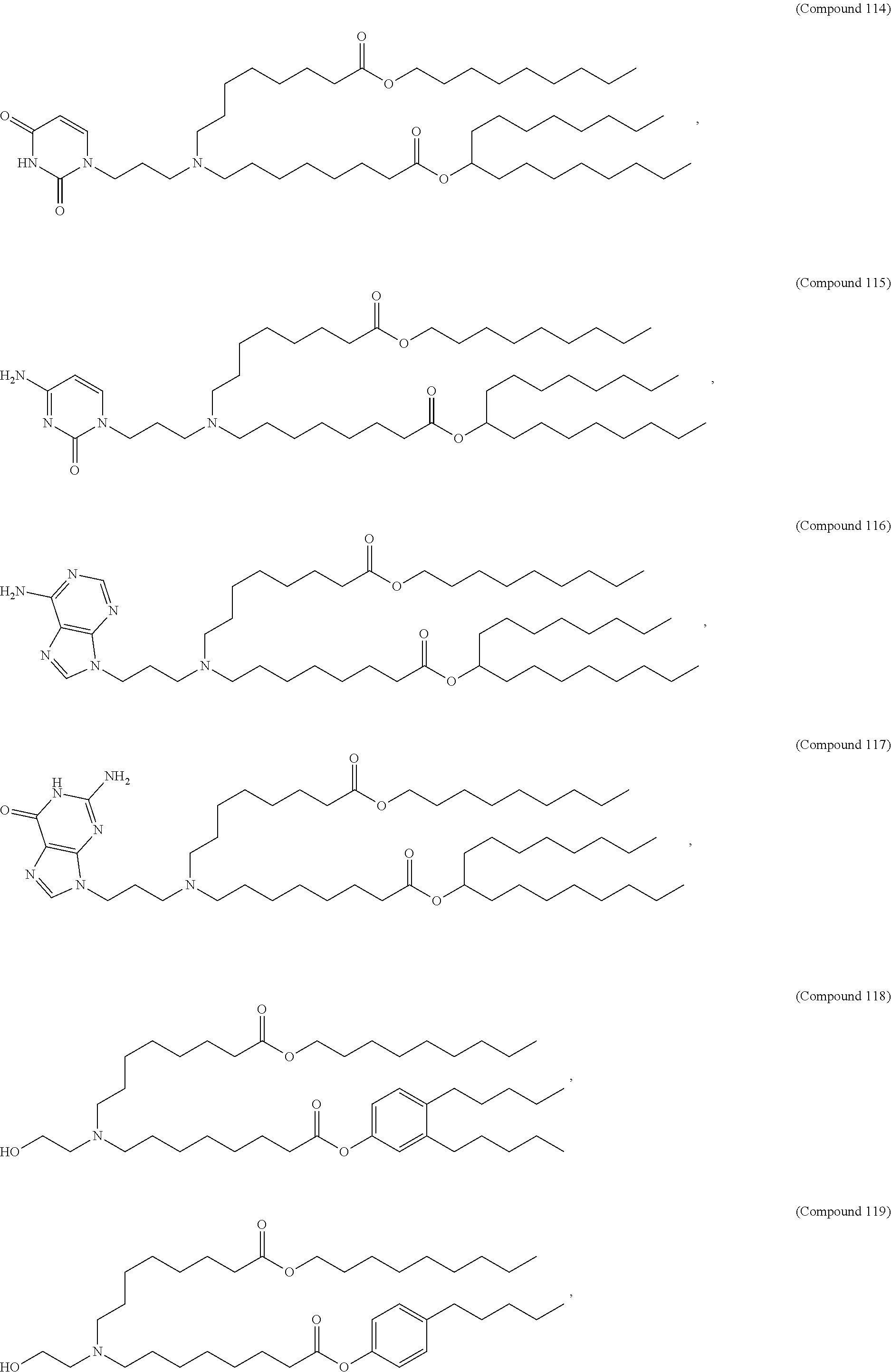

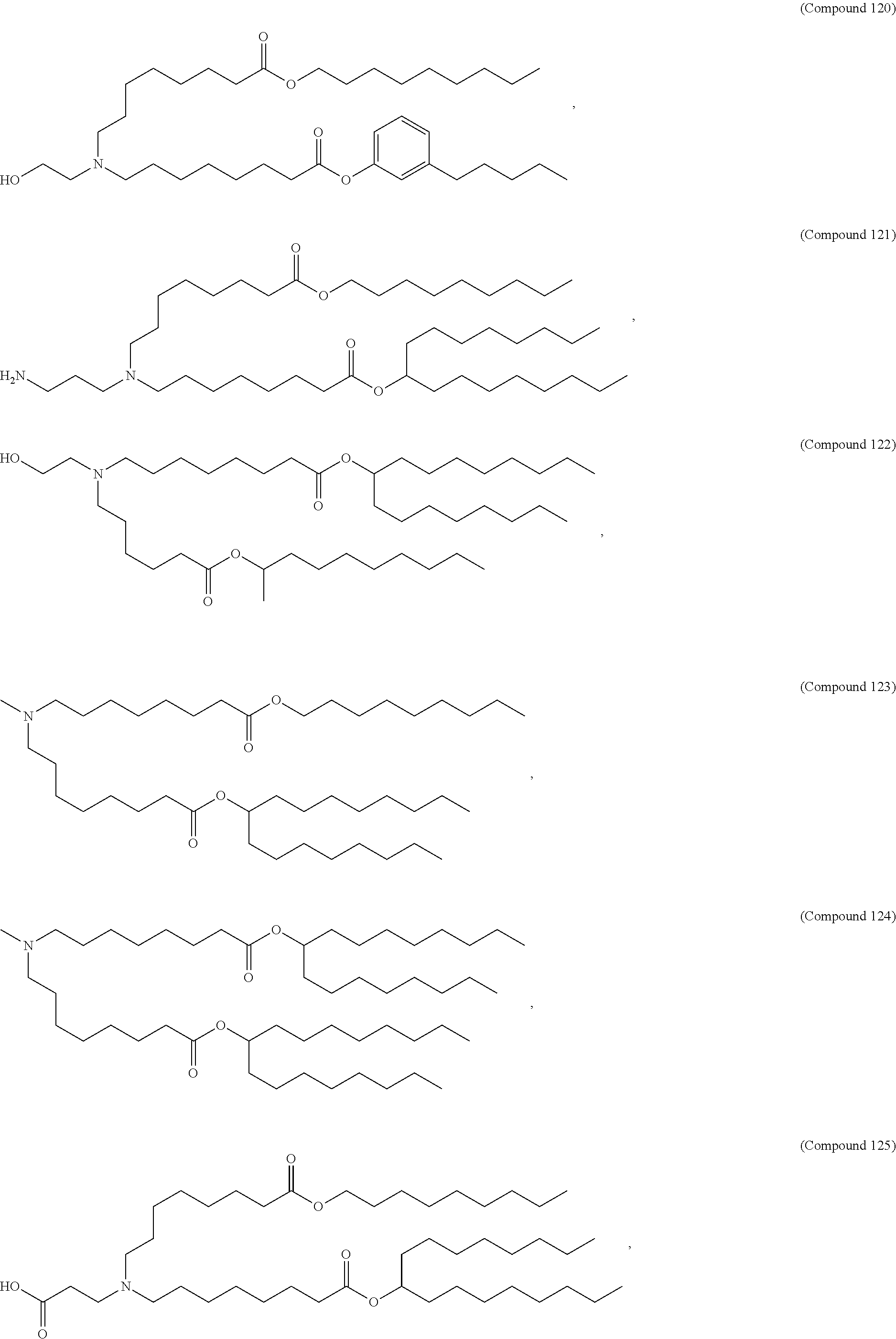

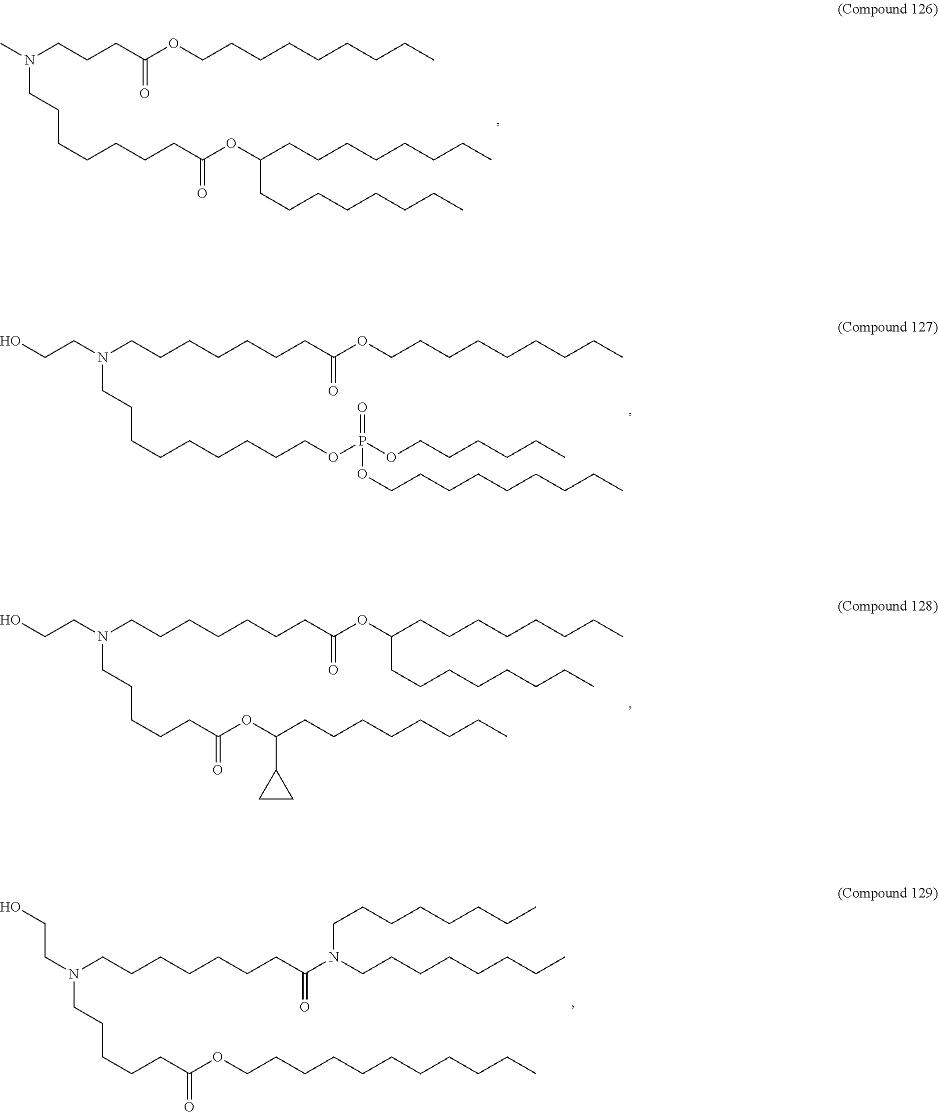

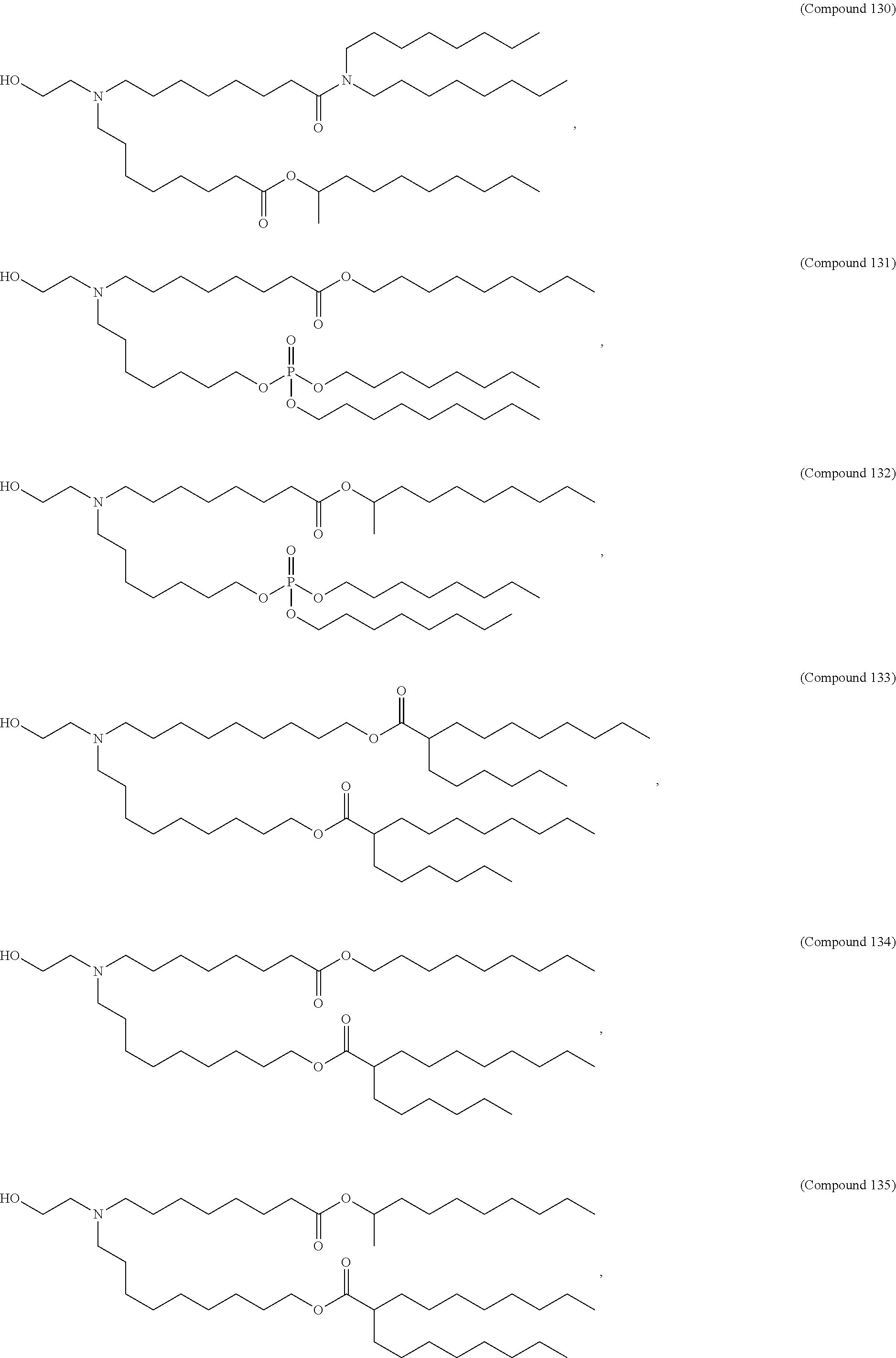

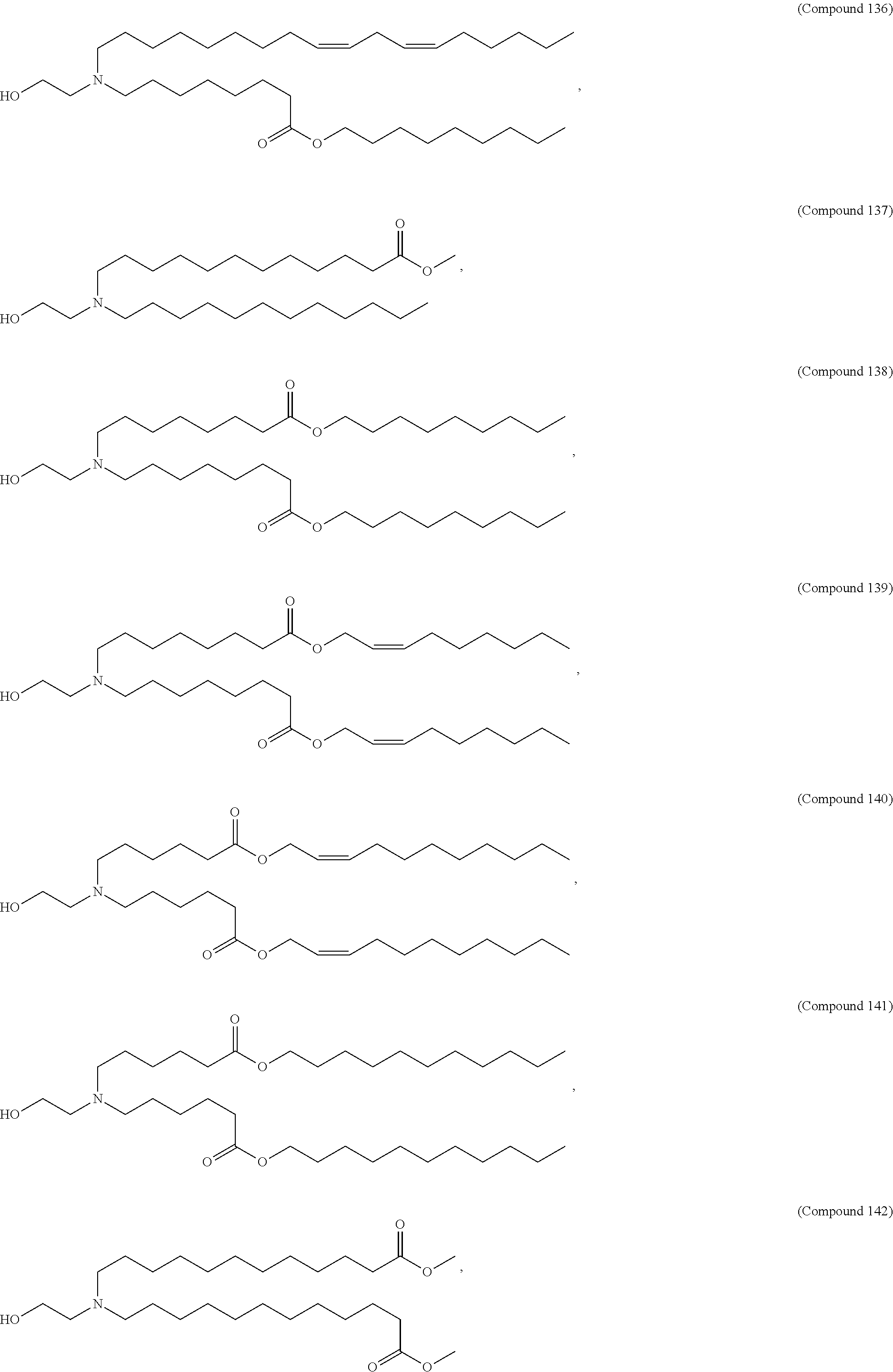

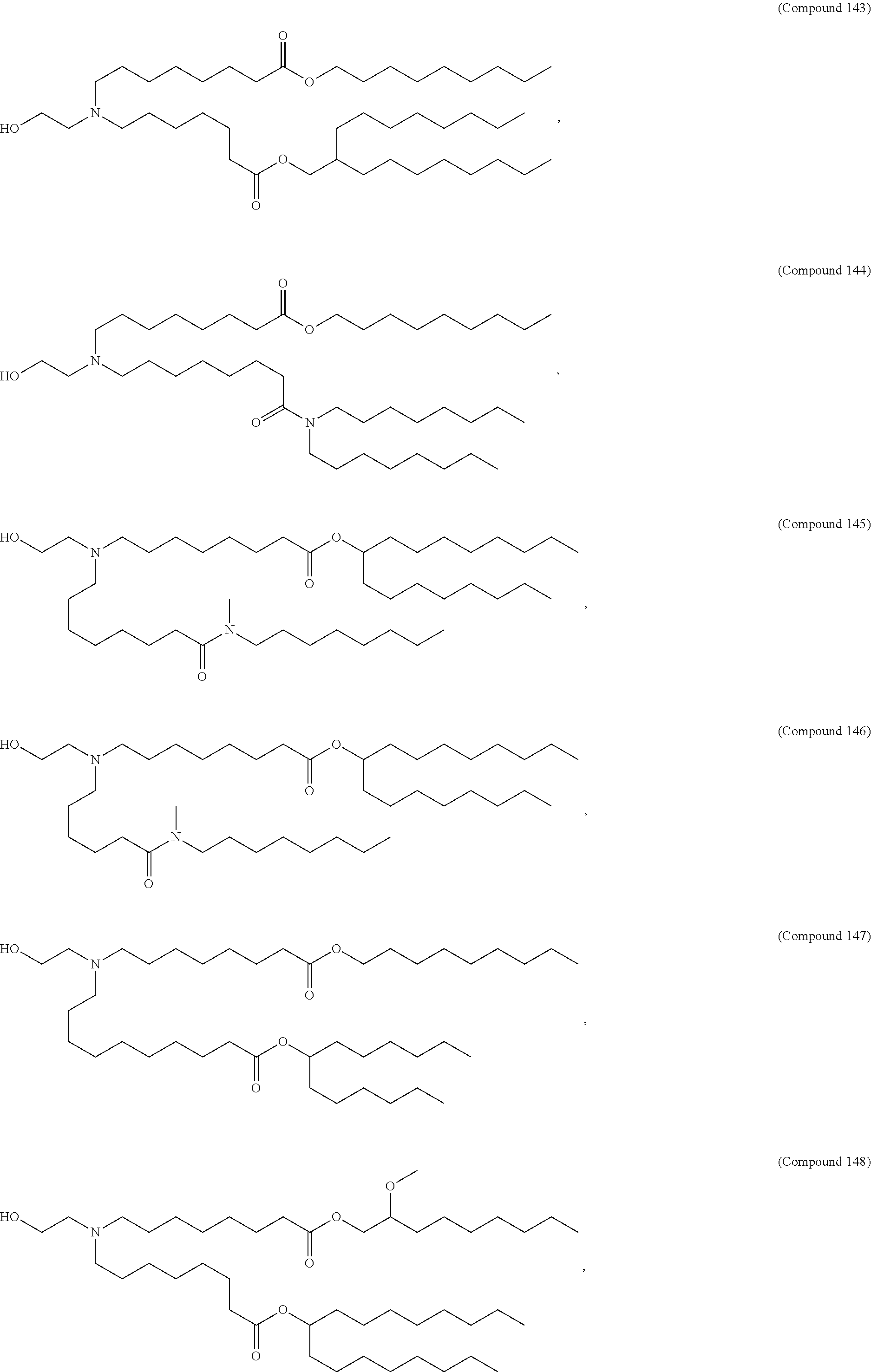

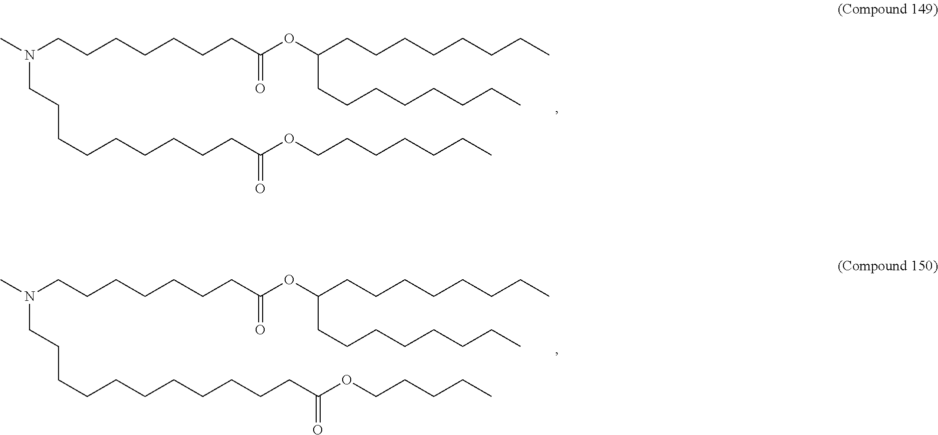

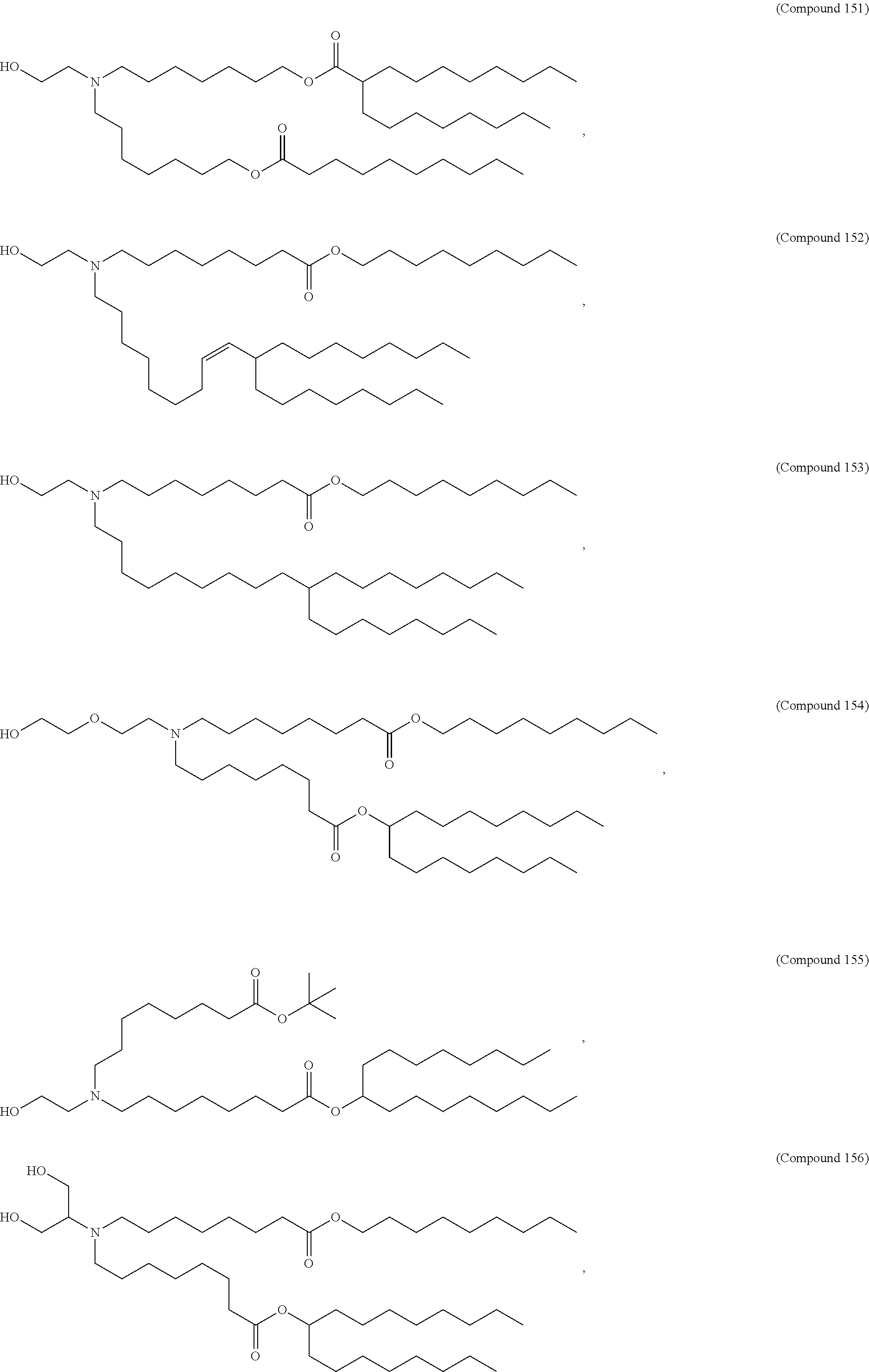

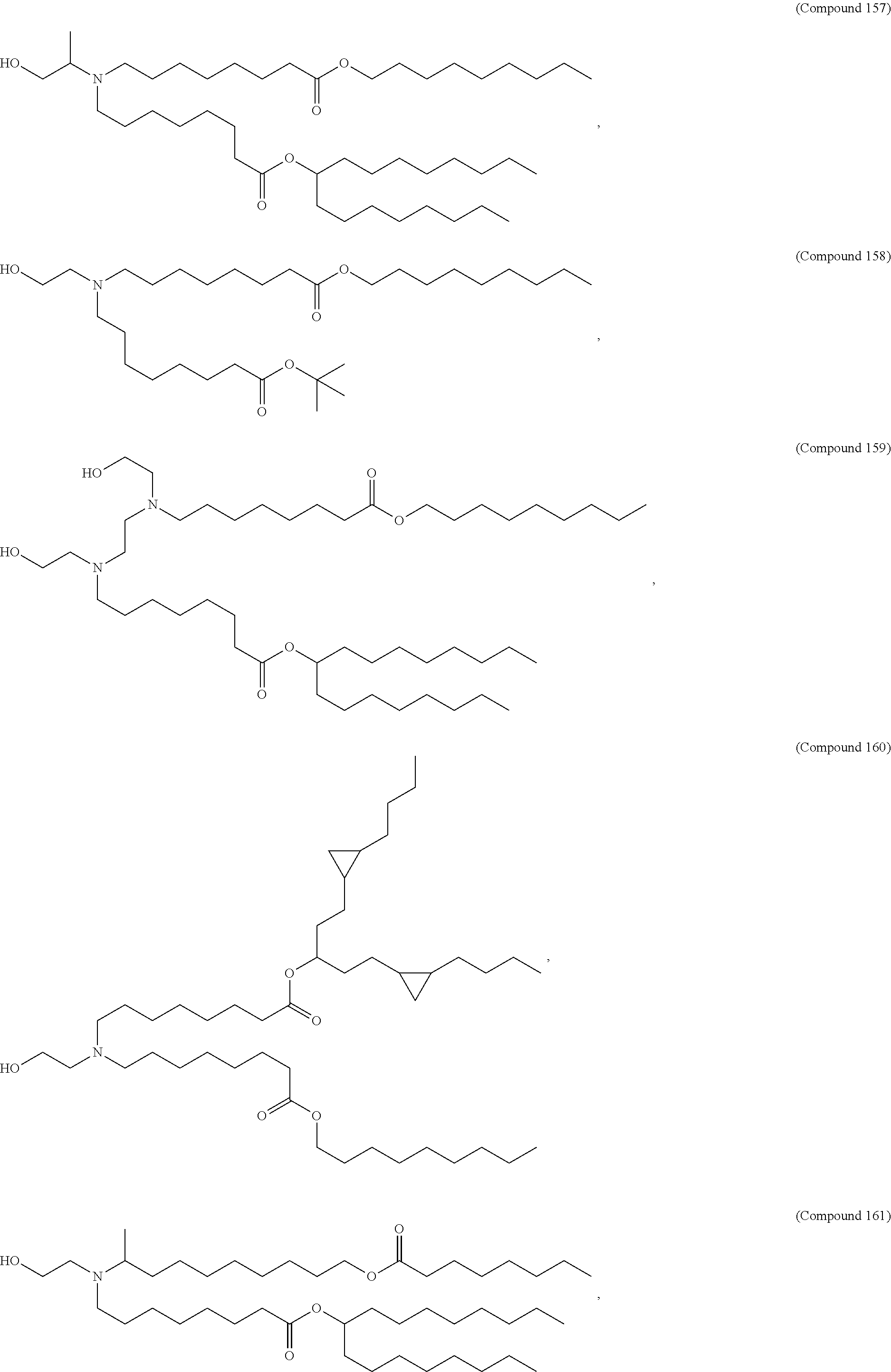

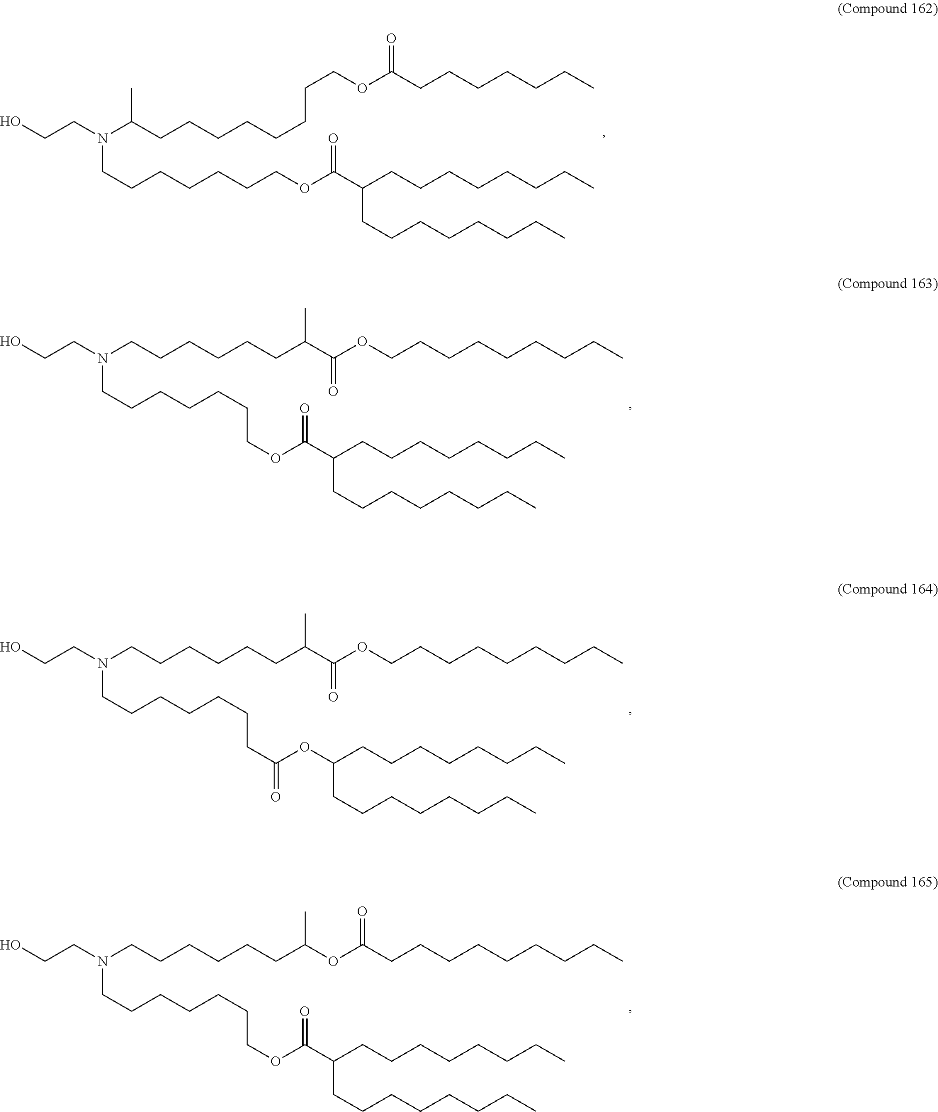

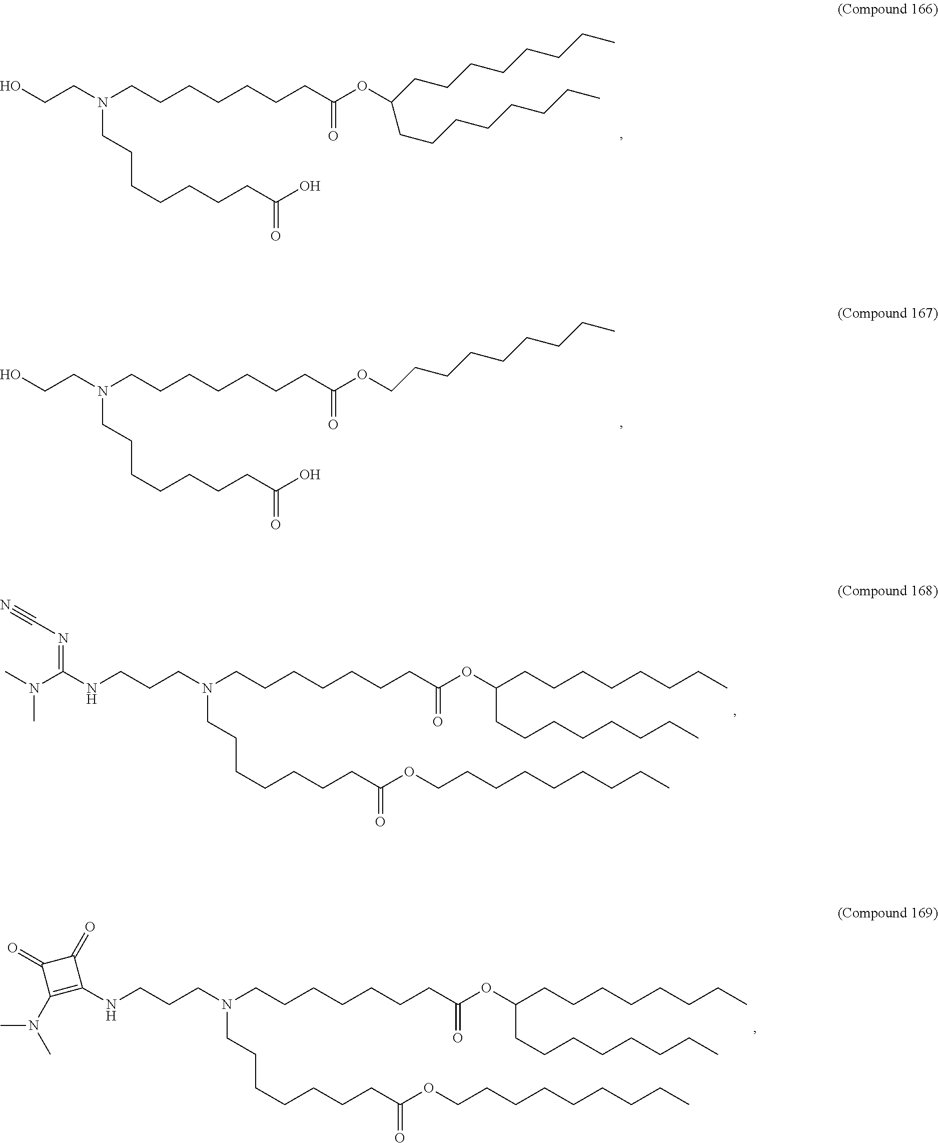

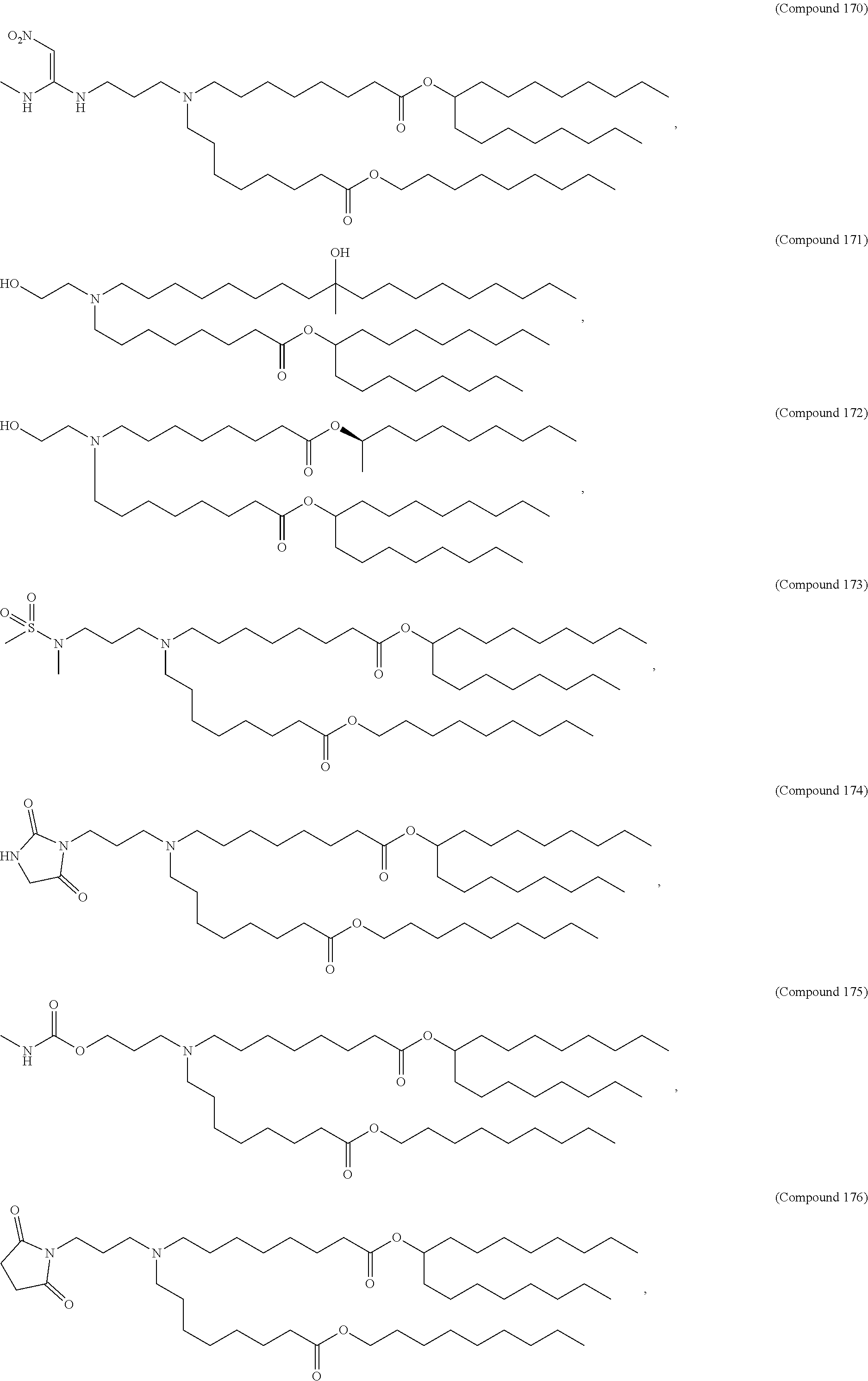

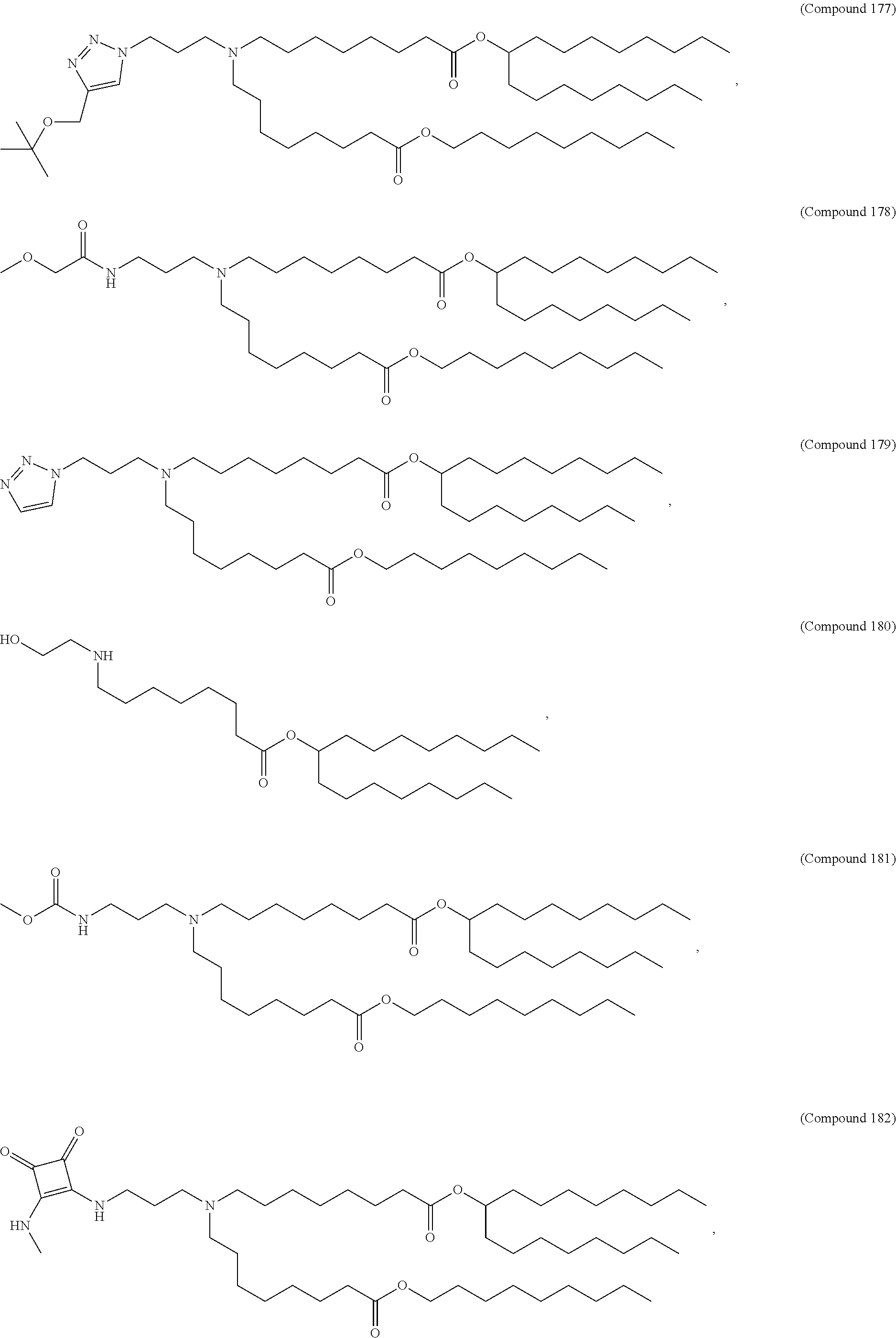

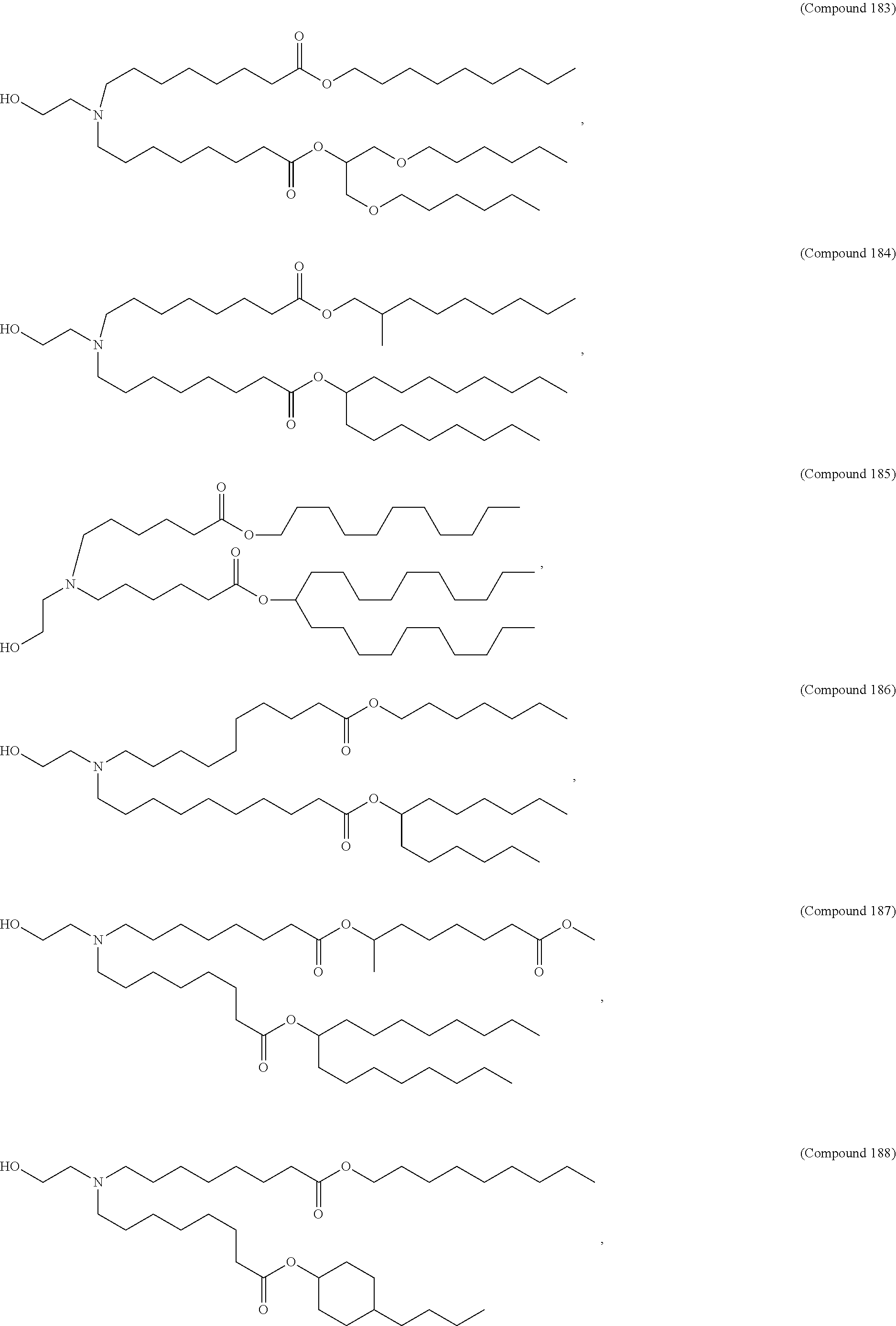

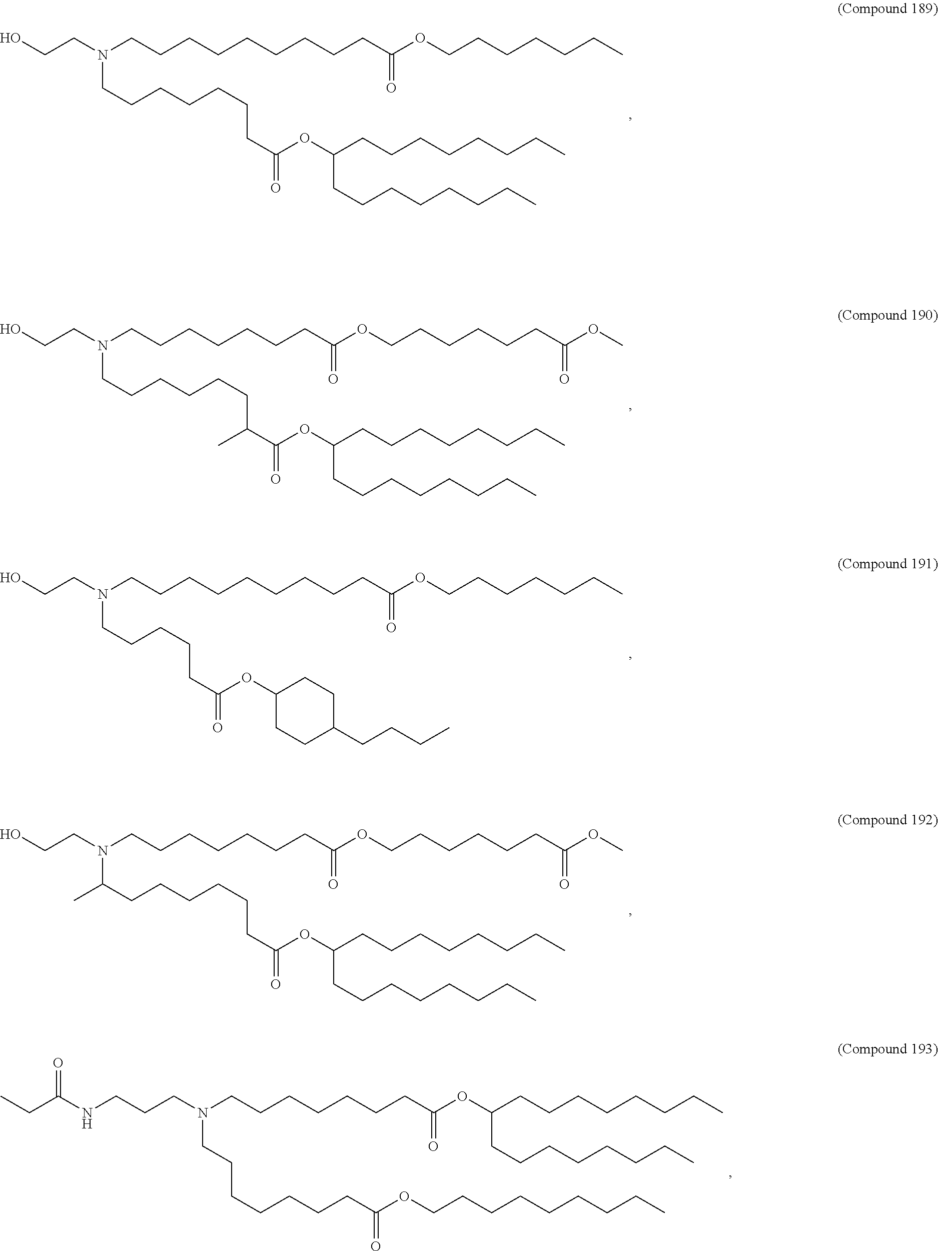

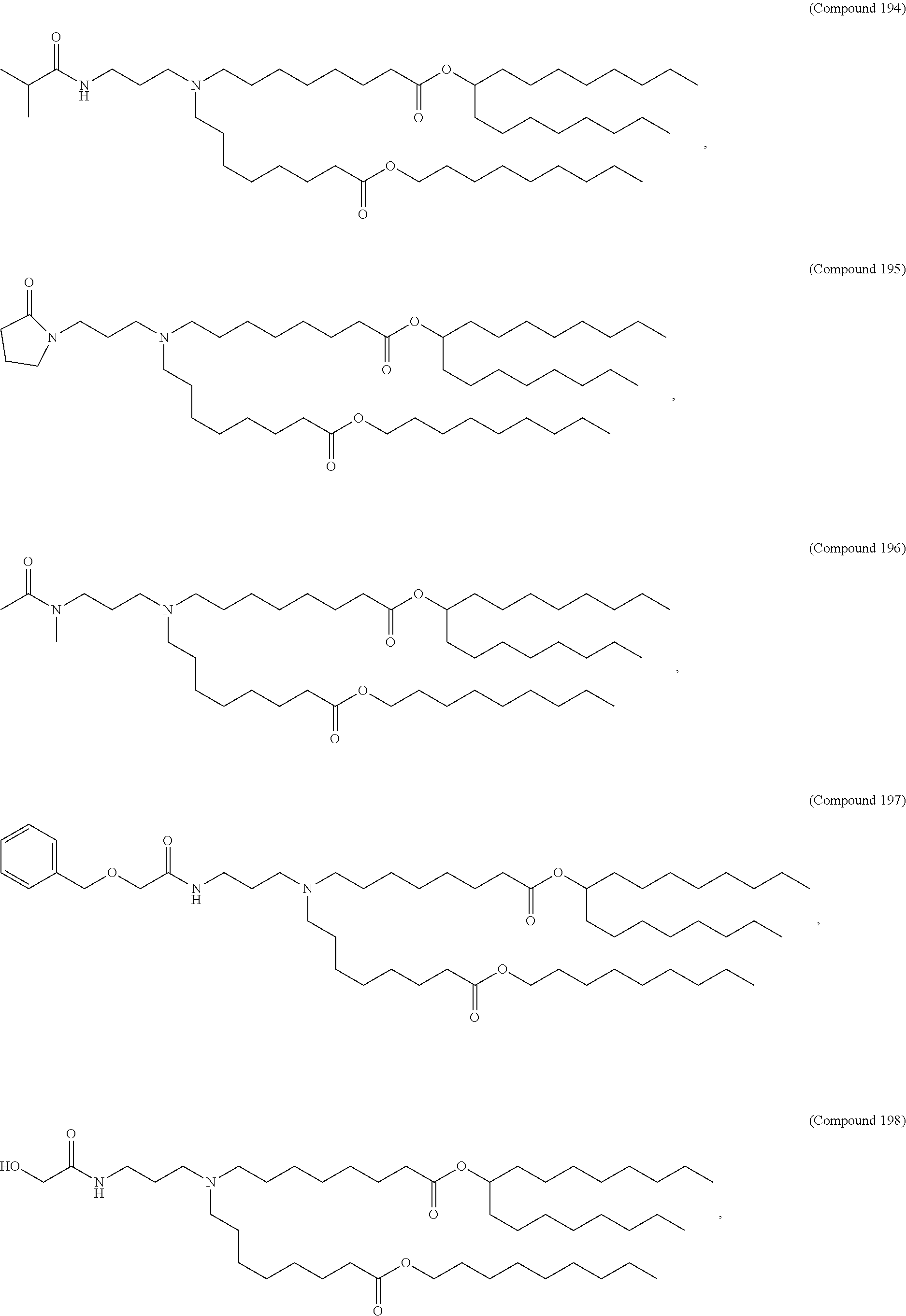

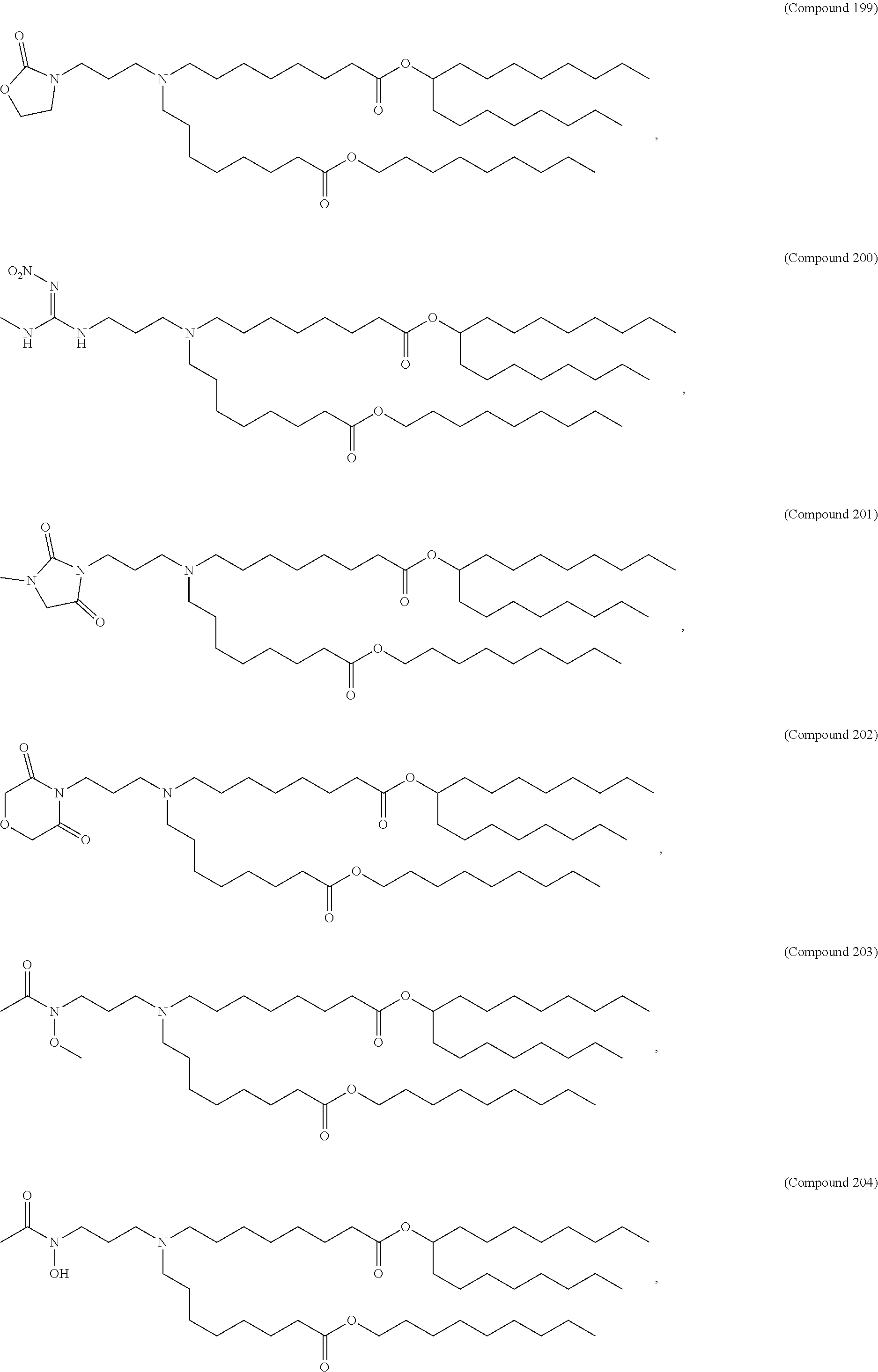

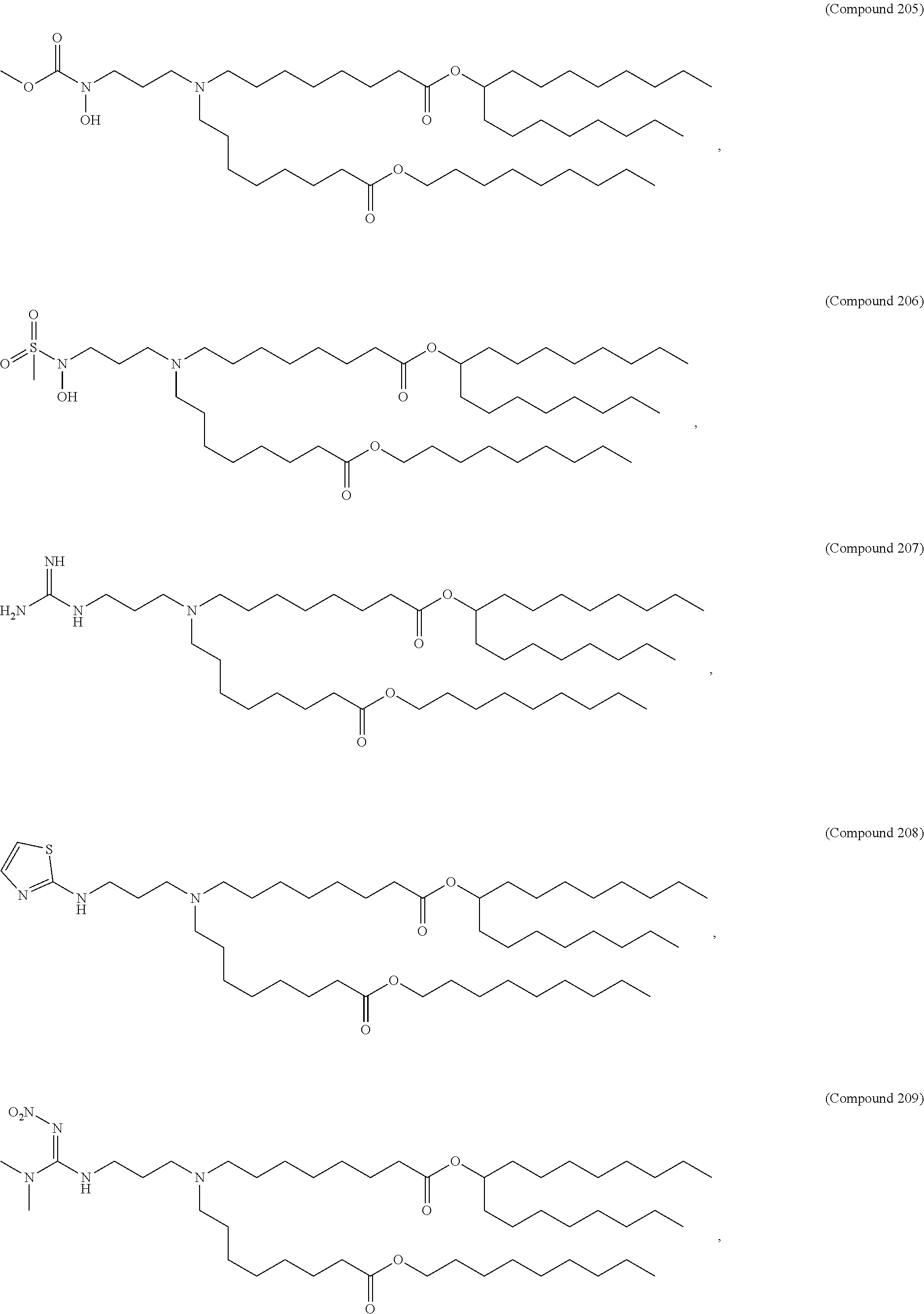

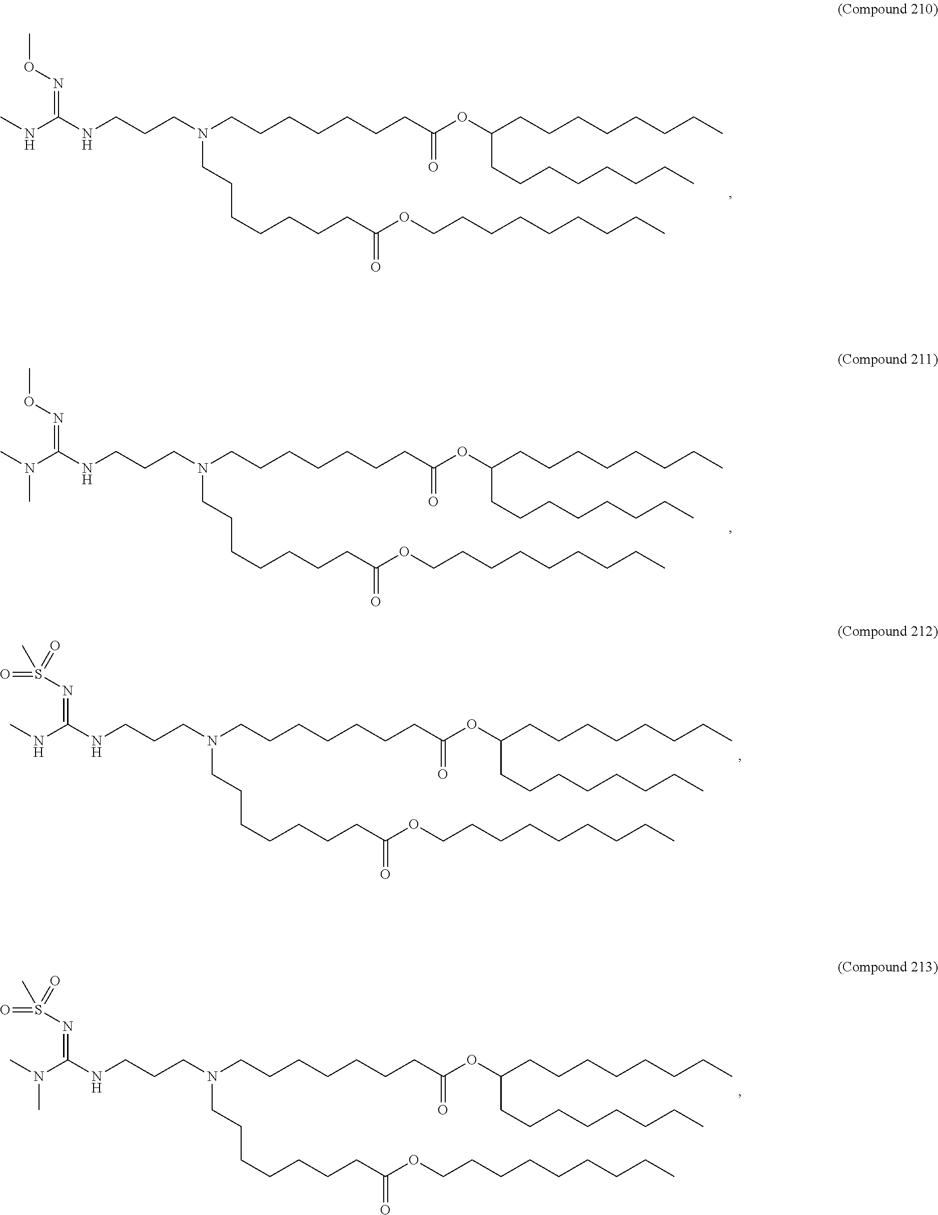

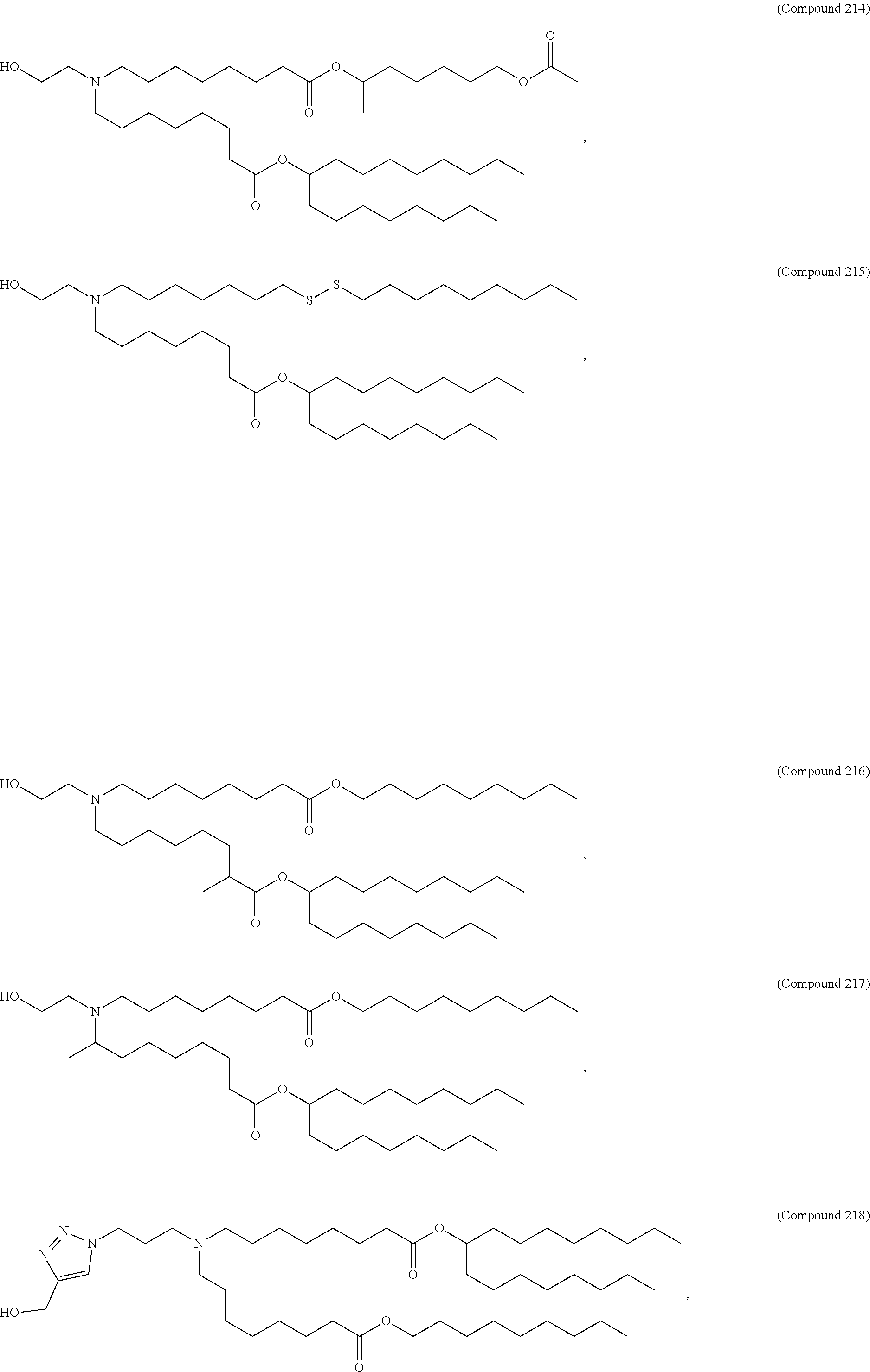

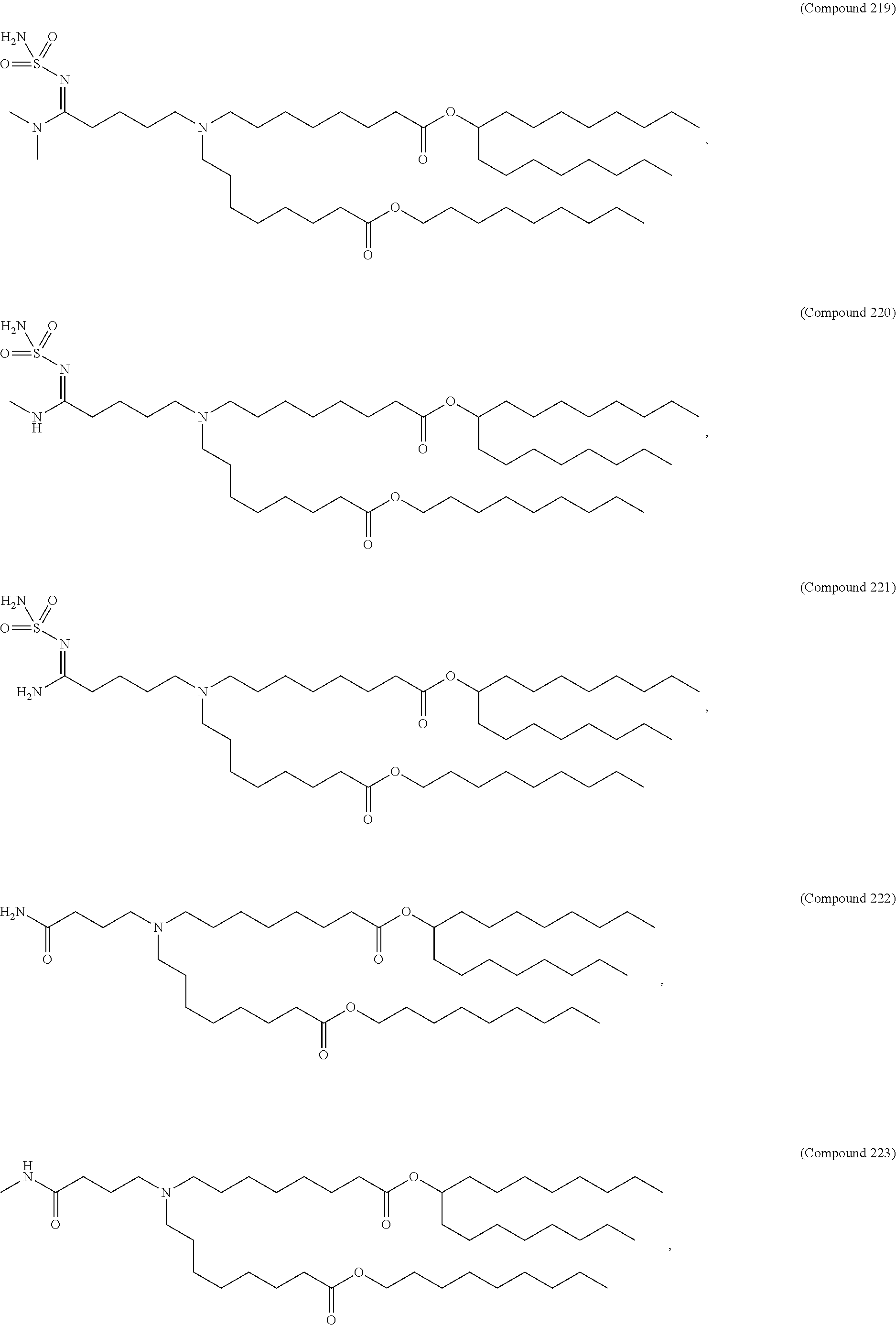

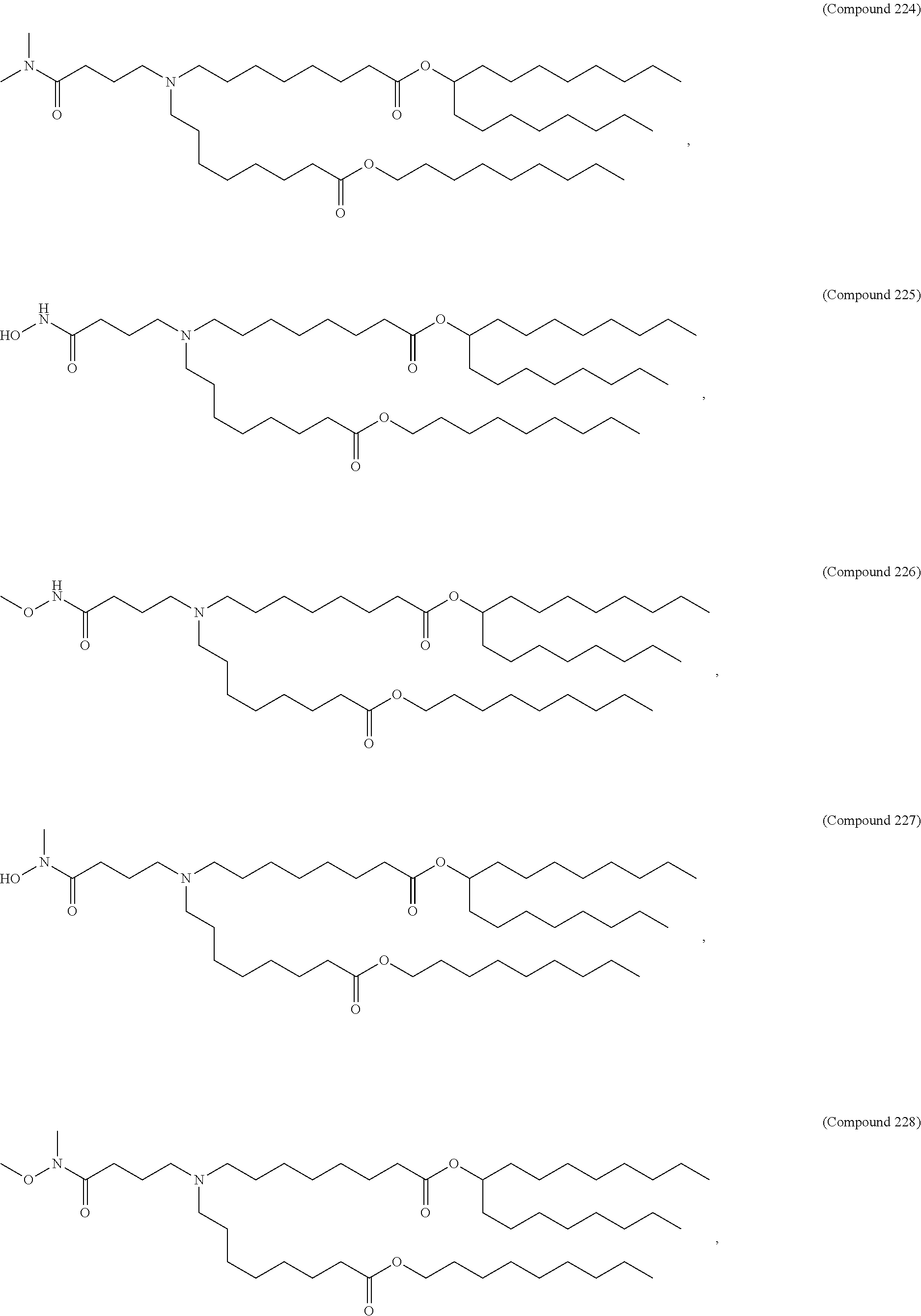

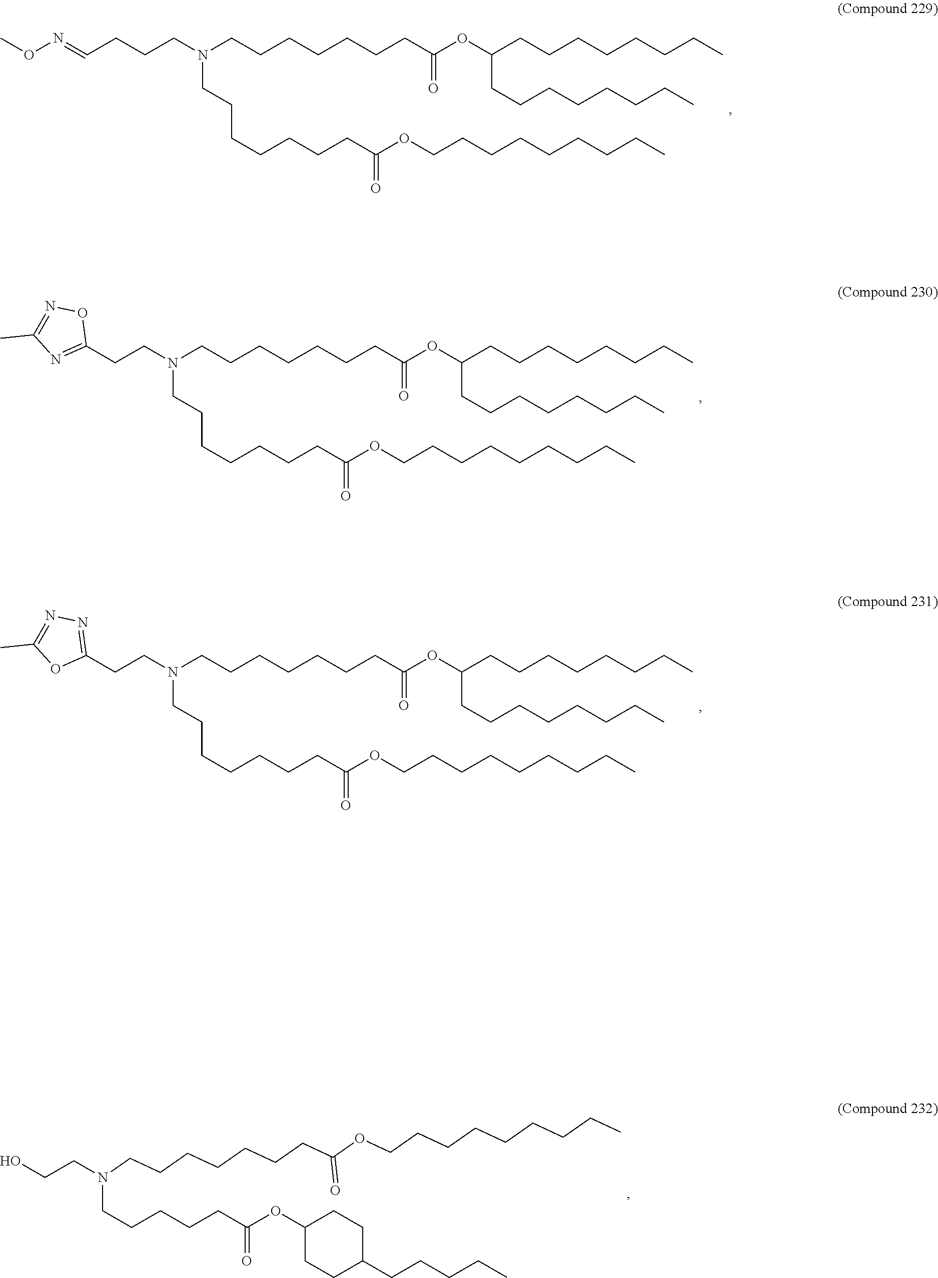

and salts or stereoisomers thereof.

In some embodiments, a nanoparticle comprises the following compound:

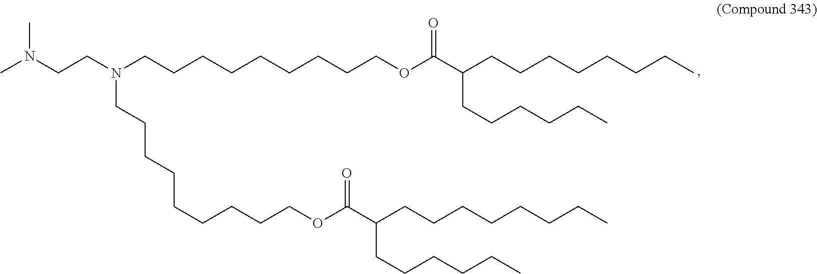 [SMILES: CCCCCCCCC(CCCCCC)C(=O)OCCCCCCCCCN(CCCCCCCCCOC(=O)C(CCCCCC)CCCCCCCC)CCN(C)C]

or salts or stereoisomers thereof.

In other embodiments, the compound of Formula (I) is selected from the group consisting of Compound 1-Compound 147, or salt or stereoisomers thereof.

In some embodiments ionizable lipids including a central piperazine moiety are provided. The lipids described herein may be advantageously used in lipid nanoparticle compositions for the delivery of therapeutic and/or prophylactic agents to mammalian cells or organs. For example, the lipids described herein have little or no immunogenicity. For example, the lipid compounds disclosed hereinhave a lower immunogenicity as compared to a reference lipid (e.g., MC3, KC2, or DLinDMA). For example, a formulation comprising a lipid disclosed herein and a therapeutic or prophylactic agent has an increased therapeutic index as compared to a corresponding formulation which comprises a reference lipid (e.g., MC3, KC2, or DLinDMA) and the same therapeutic or prophylactic agent.

In some embodiments, the delivery agent comprises a lipid compound having the formula (III)

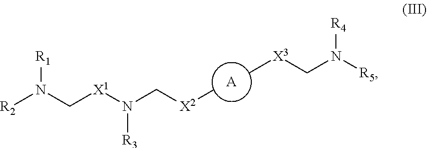

or salts or stereoisomers thereof, wherein

ring A is

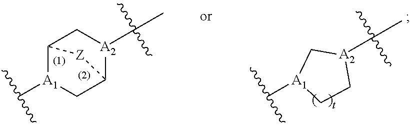

t is 1 or 2;

A₁ and A₂ are each independently selected from CH or N;

Z is CH₂ or absent wherein when Z is CH₂, the dashed lines (1) and (2) each represent a single bond; and when Z is absent, the dashed lines (1) and (2) are both absent;

R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;

each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

X¹, X², and X³ are independently selected from the group consisting of a bond, —CH₂—, —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;

each Y is independently a C₃₋₆ carbocycle;

each R* is independently selected from the group consisting of C₁₋₁₂ alkyl and C₂₋₁₂ alkenyl;

each R is independently selected from the group consisting of C₁₋₁₃ alkyl and a C₃₋₆ carbocycle;

each R′ is independently selected from the group consisting of C₁₋₁₂ alkyl, C₂₋₁₂ alkenyl, and H; and

each R″ is independently selected from the group consisting of C₃₋₁₂ alkyl and C₃₋₁₂ alkenyl,

wherein when ring A is

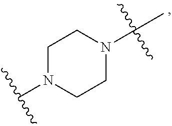 [SMILES: *N1CCN(*)CC1]

then

i) at least one of X¹, X², and X³ is not —CH₂—; and/or

ii) at least one of R₁, R₂, R₃, R₄, and R₅ is —R″MR′.

In some embodiments, the compound is of any of formulae (IIIa1)-(IIIa6):

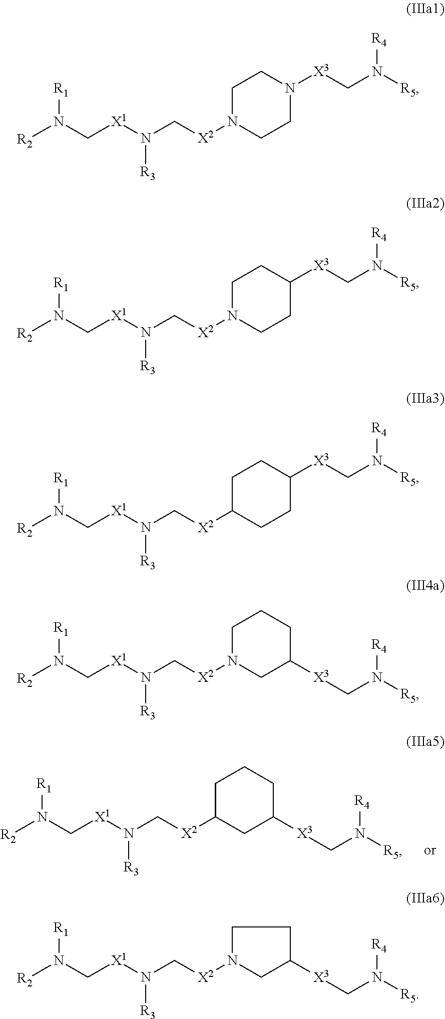

The compounds of Formula (III) or any of (IIIa1)-(IIIa6) include one or more of the following features when applicable.

In some embodiments, ring A is

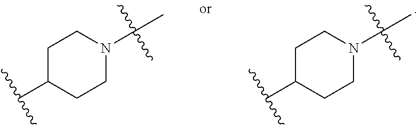 [SMILES: *C1CCN(*)CC1 ; *C1CCN(*)CC1]

In some embodiments, ring A is

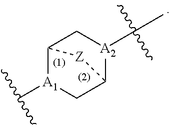

In some embodiments, ring is

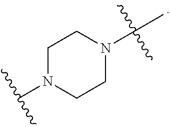 [SMILES: *N1CCN(*)CC1]

In some embodiments, ring A is

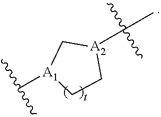

In some embodiments, ring A is

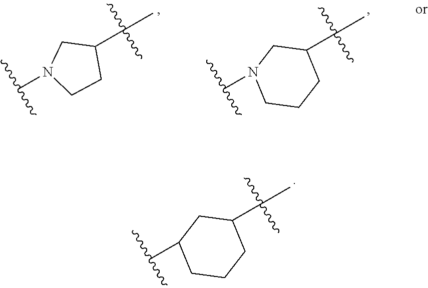 [SMILES: *C1CCN(*)C1 ; *C1CCCN(*)C1 ; *C1CCCC(*)C1]

In some embodiments, ring A is

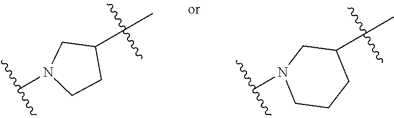 [SMILES: *C1CCN(*)C1 ; *C1CCCN(*)C1]

wherein ring, in which the N atom is connected with X².

In some embodiments, Z is CH₂.

In some embodiments, Z is absent.

In some embodiments, at least one of A₁ and A₂ is N.

In some embodiments, each of A₁ and A₂ is N.

In some embodiments, each of A₁ and A₂ is CH.

In some embodiments, A₁ is N and A₂ is CH.

In some embodiments, A₁ is CH and A₂ is N.

In some embodiments, at least one of X¹, X², and X³ is not —CH₂—. For example, in certain embodiments, X¹ is not —CH₂—. In some embodiments, at least one of X¹, X², and X³ is —C(O)—.

In some embodiments, X² is —C(O)—, —C(O)O—, —OC(O)—, —C(O)—CH₂—, —CH₂—C(O)—, —C(O)O—CH₂—, —OC(O)—CH₂—, —CH₂—C(O)O—, or —CH₂—OC(O)—.

In some embodiments, X³ is —C(O)—, —C(O)O—, —OC(O)—, —C(O)—CH₂—, —CH₂—C(O)—, —C(O)O—CH₂—, —OC(O)—CH₂—, —CH₂—C(O)O—, or —CH₂—OC(O)—. In other embodiments, X³ is —CH₂—.

In some embodiments, X³ is a bond or —(CH₂)₂—.

In some embodiments, R₁ and R₂ are the same. In certain embodiments, R₁, R₂, and R₃ are the same. In some embodiments, R₄ and R₅ are the same. In certain embodiments, R₁, R₂, R₃, R₄, and R₅ are the same.

In some embodiments, at least one of R₁, R₂, R₃, R₄, and R₅ is —R″MR′. In some embodiments, at most one of R₁, R₂, R₃, R₄, and R₅ is —R″MR′. For example, at least one of R₁, R₂, and R₃ may be —R″MR′, and/or at least one of R₄ and R₅ is —R″MR′. In certain embodiments, at least one M is —C(O)O—. In some embodiments, each M is —C(O)O—. In some embodiments, at least one M is —OC(O)—. In some embodiments, each M is —OC(O)—. In some embodiments, at least one M is —OC(O)O—. In some embodiments, each M is —OC(O)O—. In some embodiments, at least one R″ is C₃ alkyl. In certain embodiments, each R″ is C₃ alkyl. In some embodiments, at least one R″ is C₅ alkyl. In certain embodiments, each R″ is C₅ alkyl. In some embodiments, at least one R″ is C₆ alkyl. In certain embodiments, each R″ is C₆ alkyl. In some embodiments, at least one R″ is C₇ alkyl. In certain embodiments, each R″ is C₇ alkyl. In some embodiments, at least one R′ is C₅ alkyl. In certain embodiments, each R′ is C₅ alkyl. In other embodiments, at least one R′ is C₁ alkyl. In certain embodiments, each R′ is C₁ alkyl. In some embodiments, at least one R′ is C₂ alkyl. In certain embodiments, each R′ is C₂ alkyl.

In some embodiments, at least one of R₁, R₂, R₃, R₄, and R₅ is C₁₂ alkyl. In certain embodiments, each of R₁, R₂, R₃, R₄, and R₅ are C₁₂ alkyl.

In certain embodiments, the compound is selected from the group consisting of:

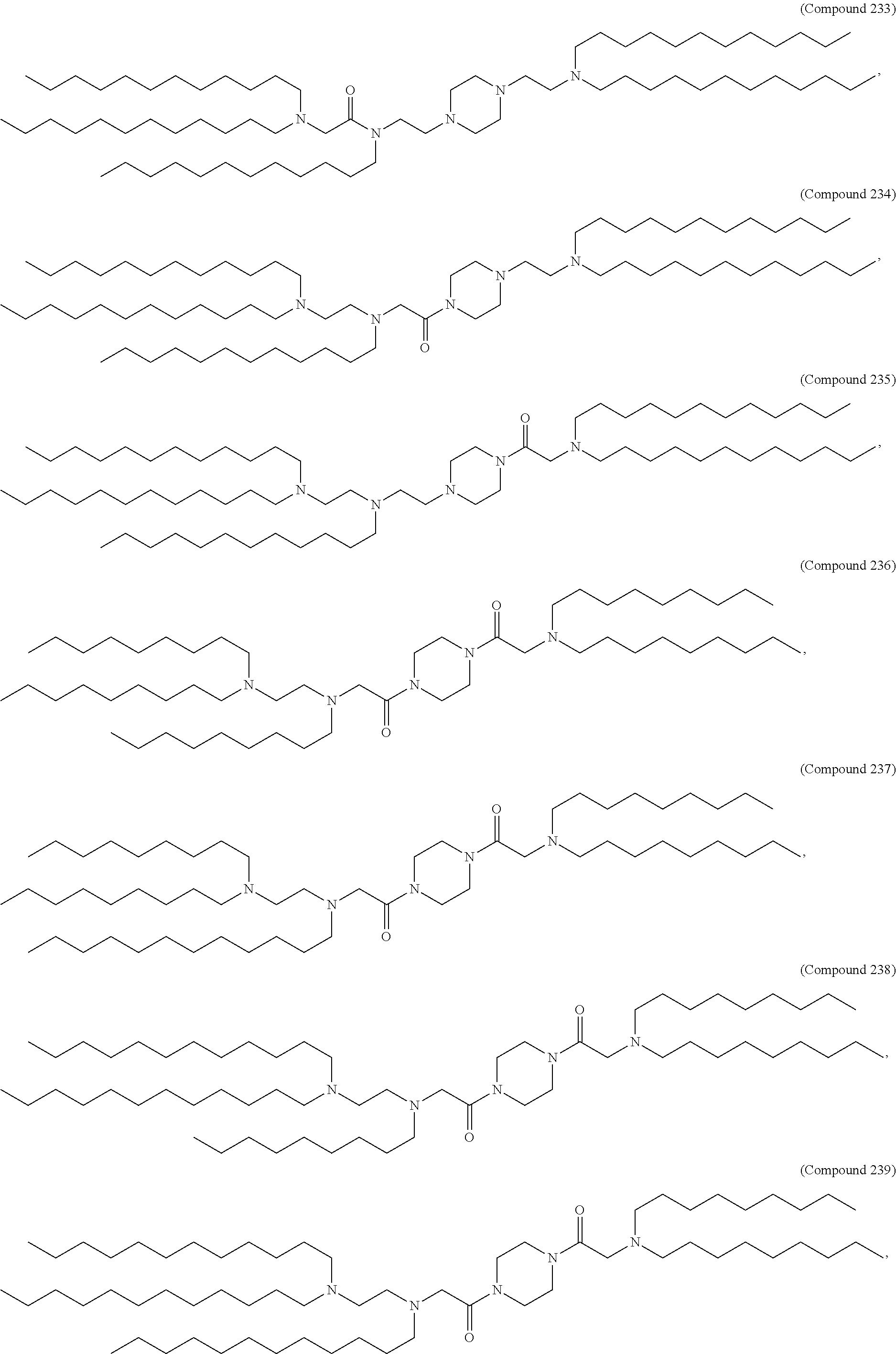

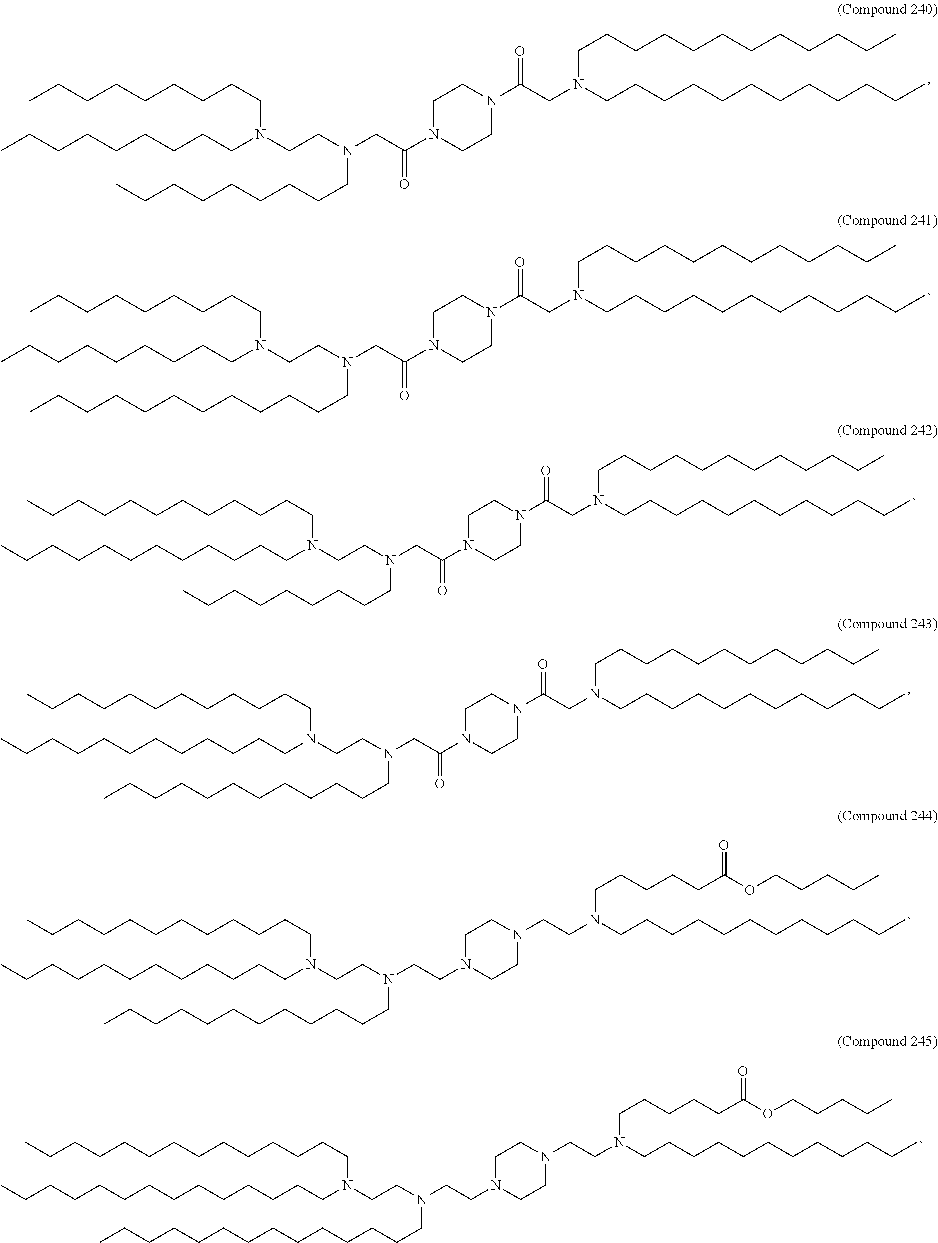

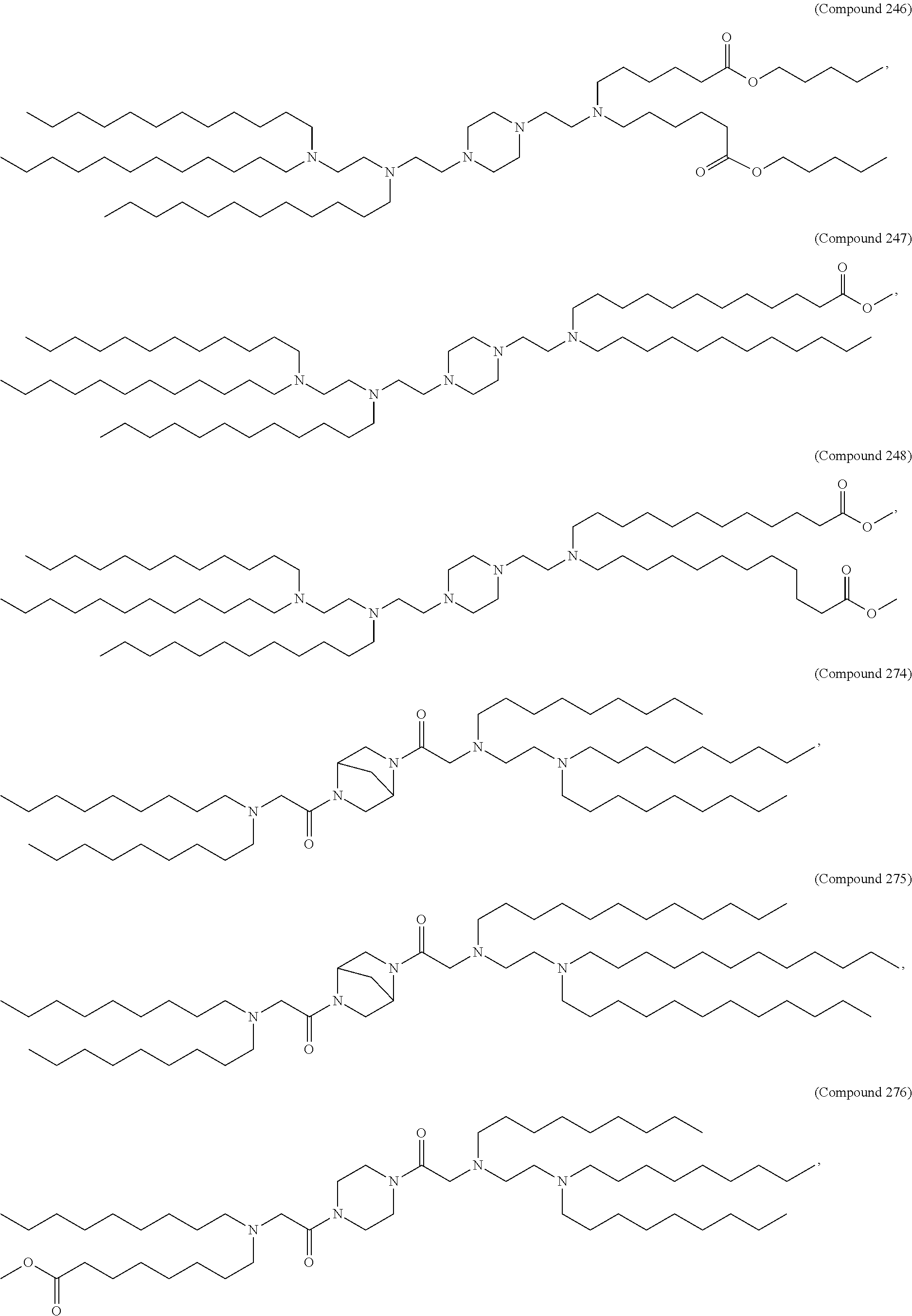

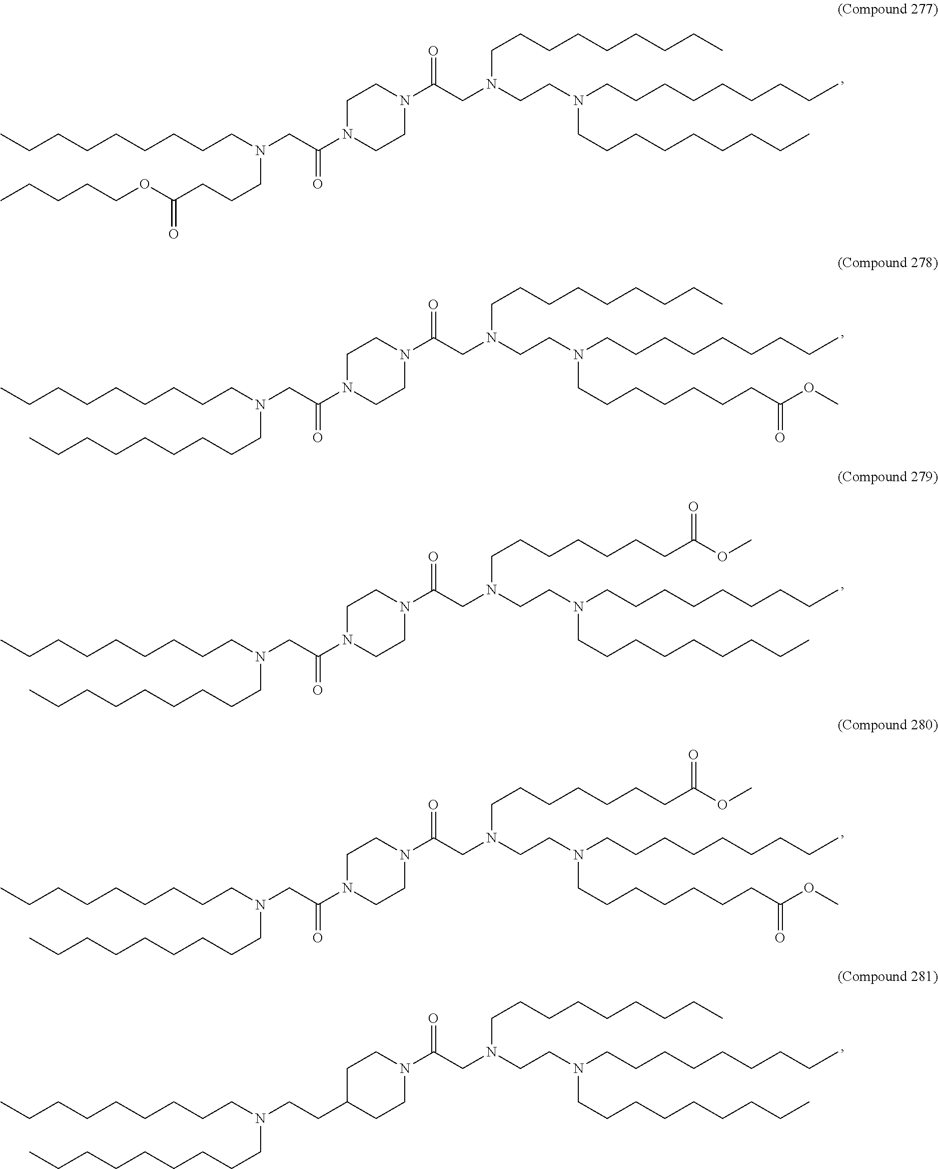

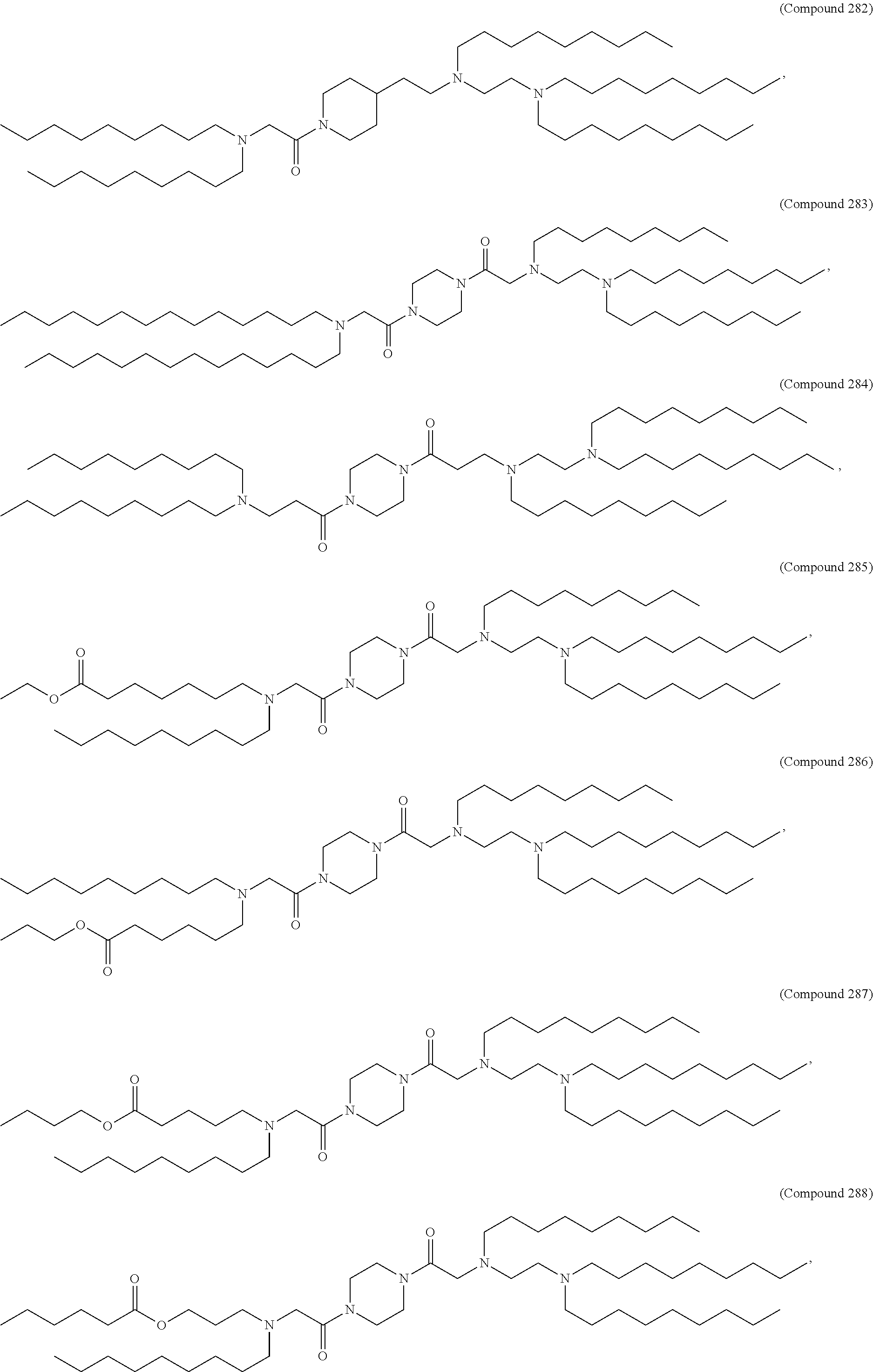

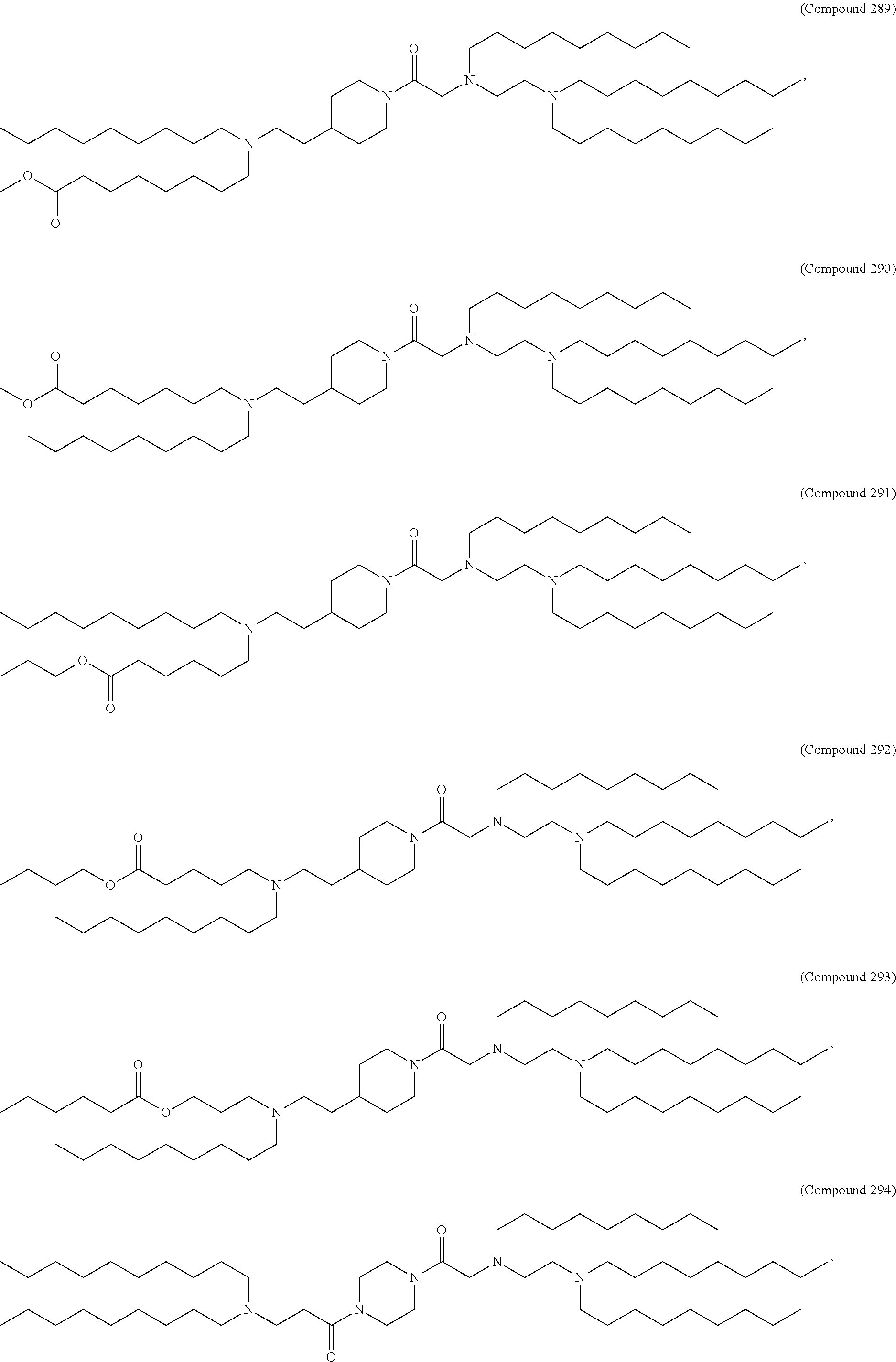

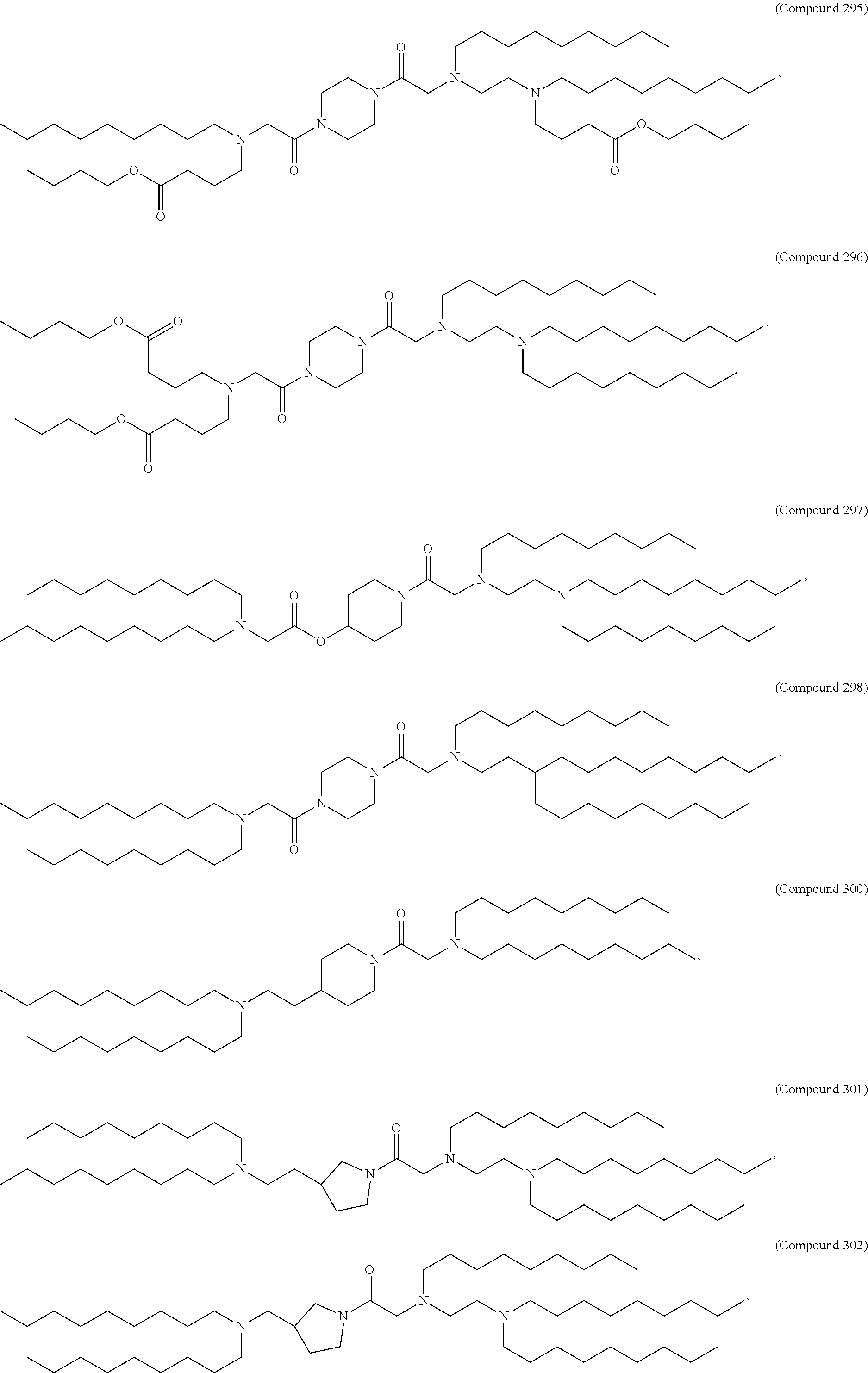

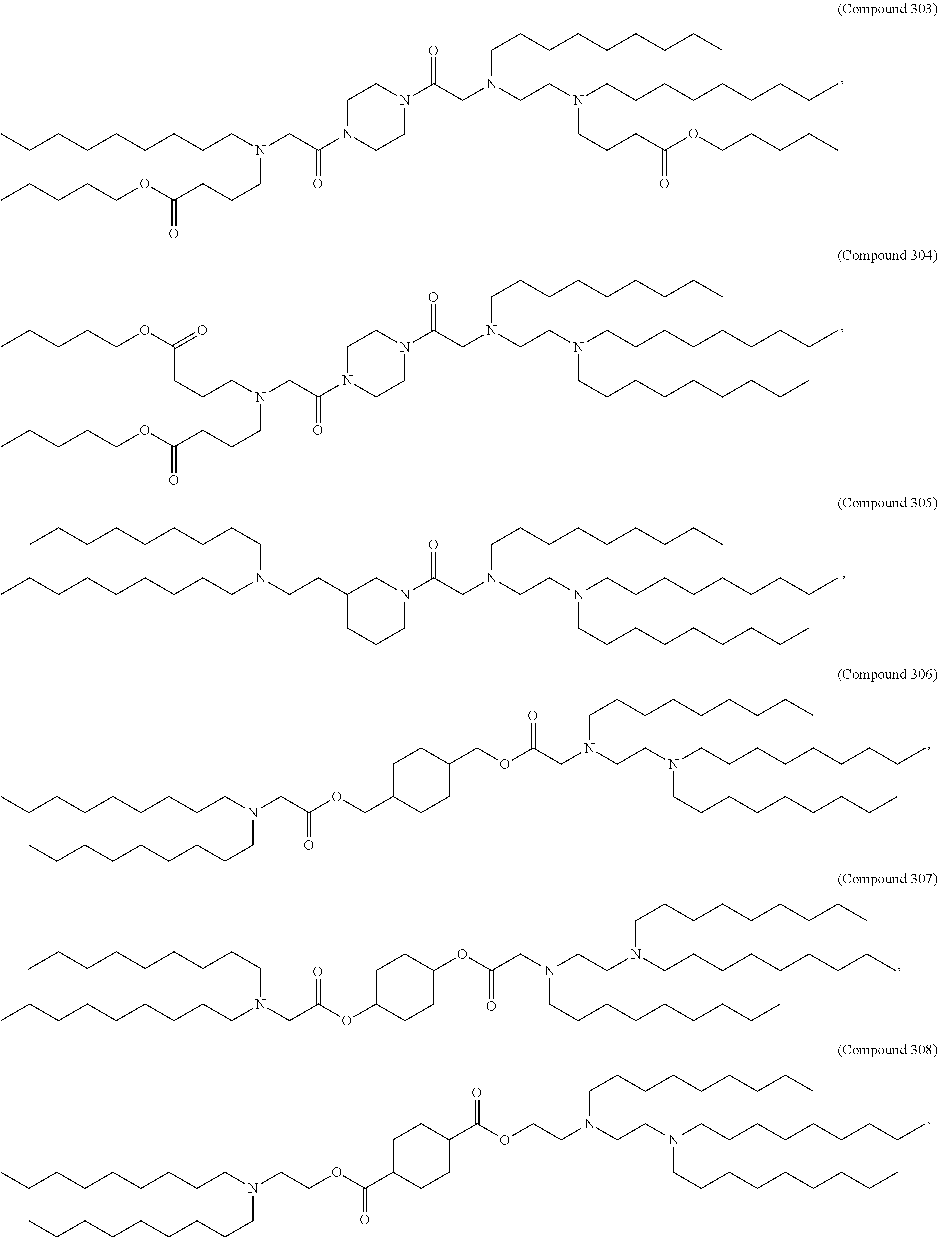

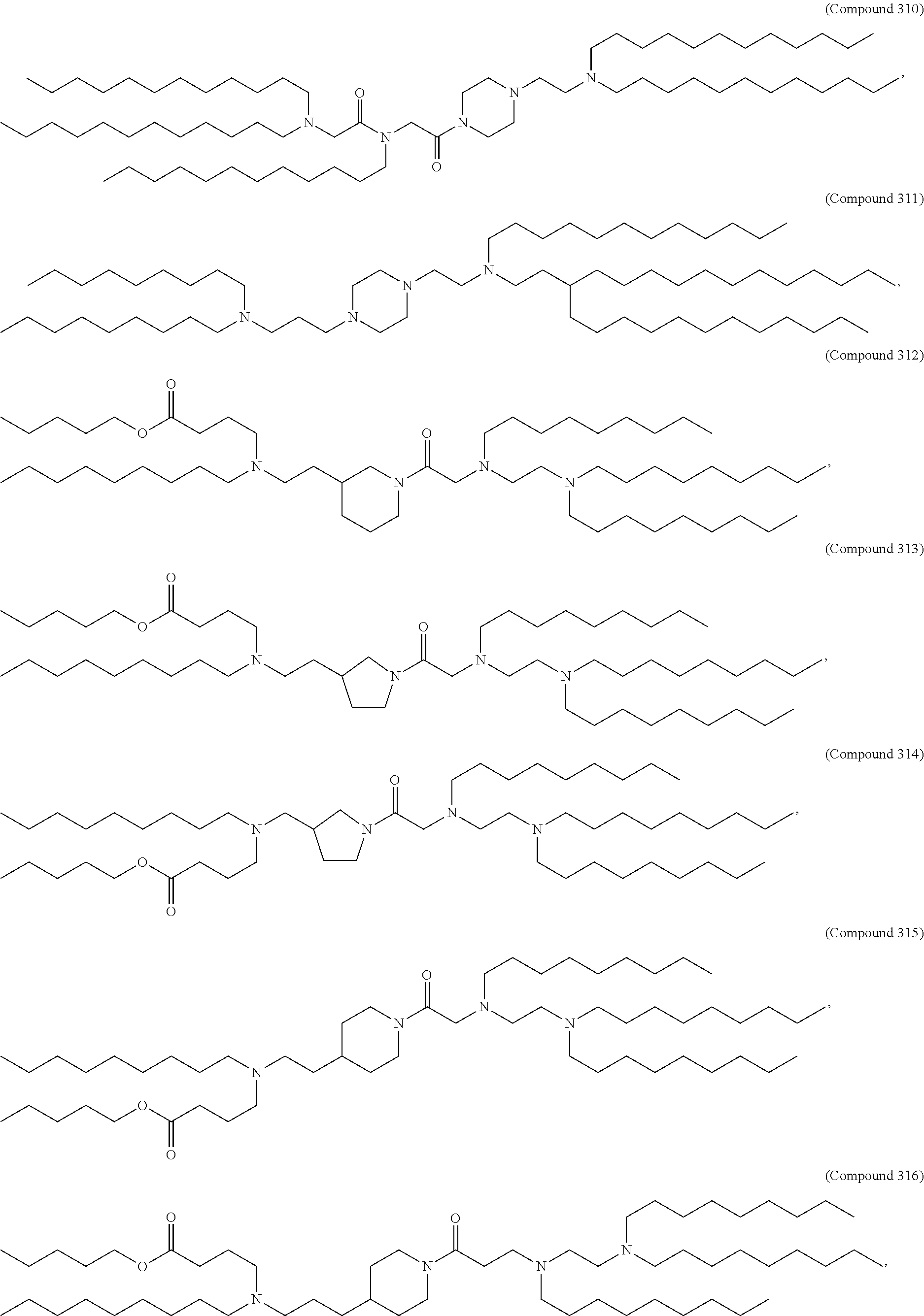

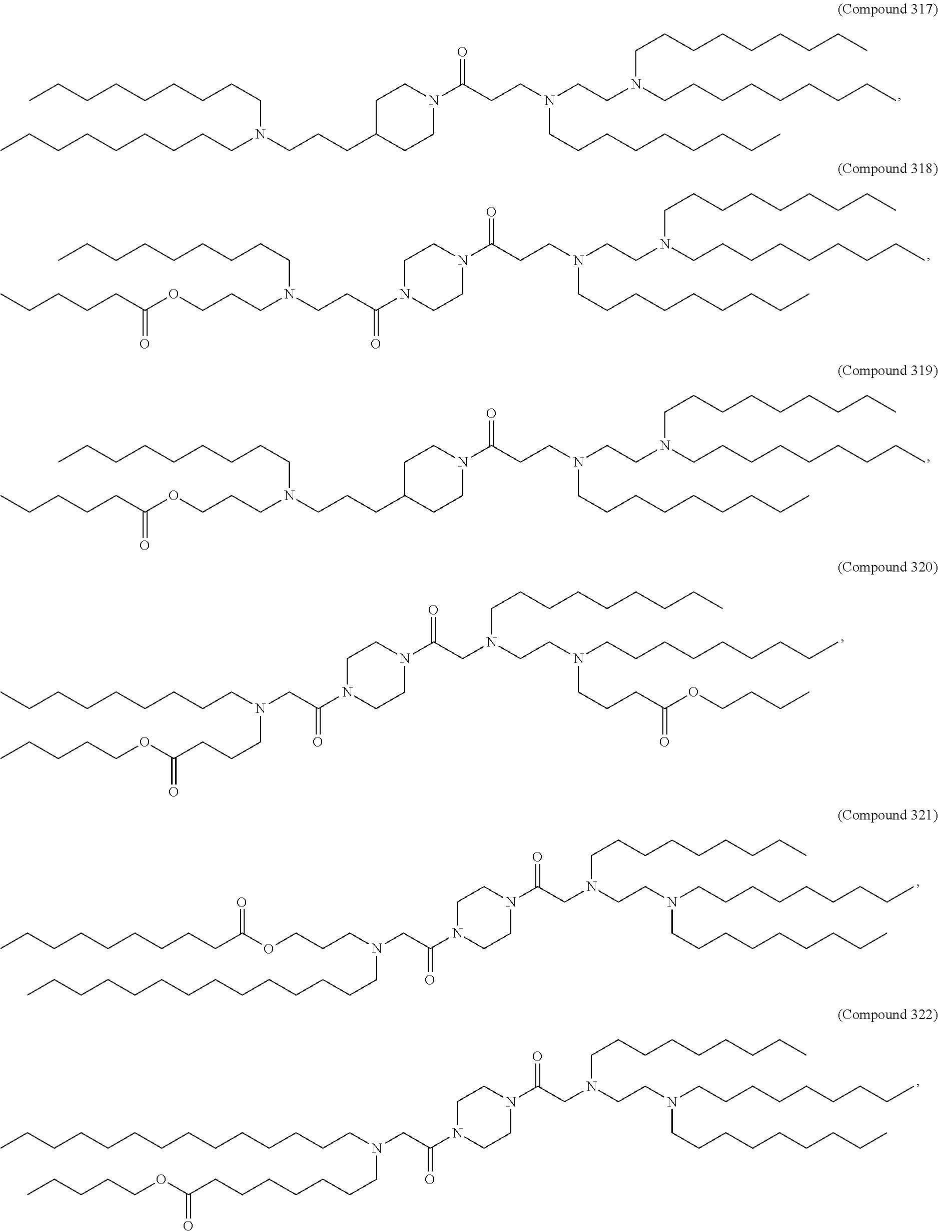

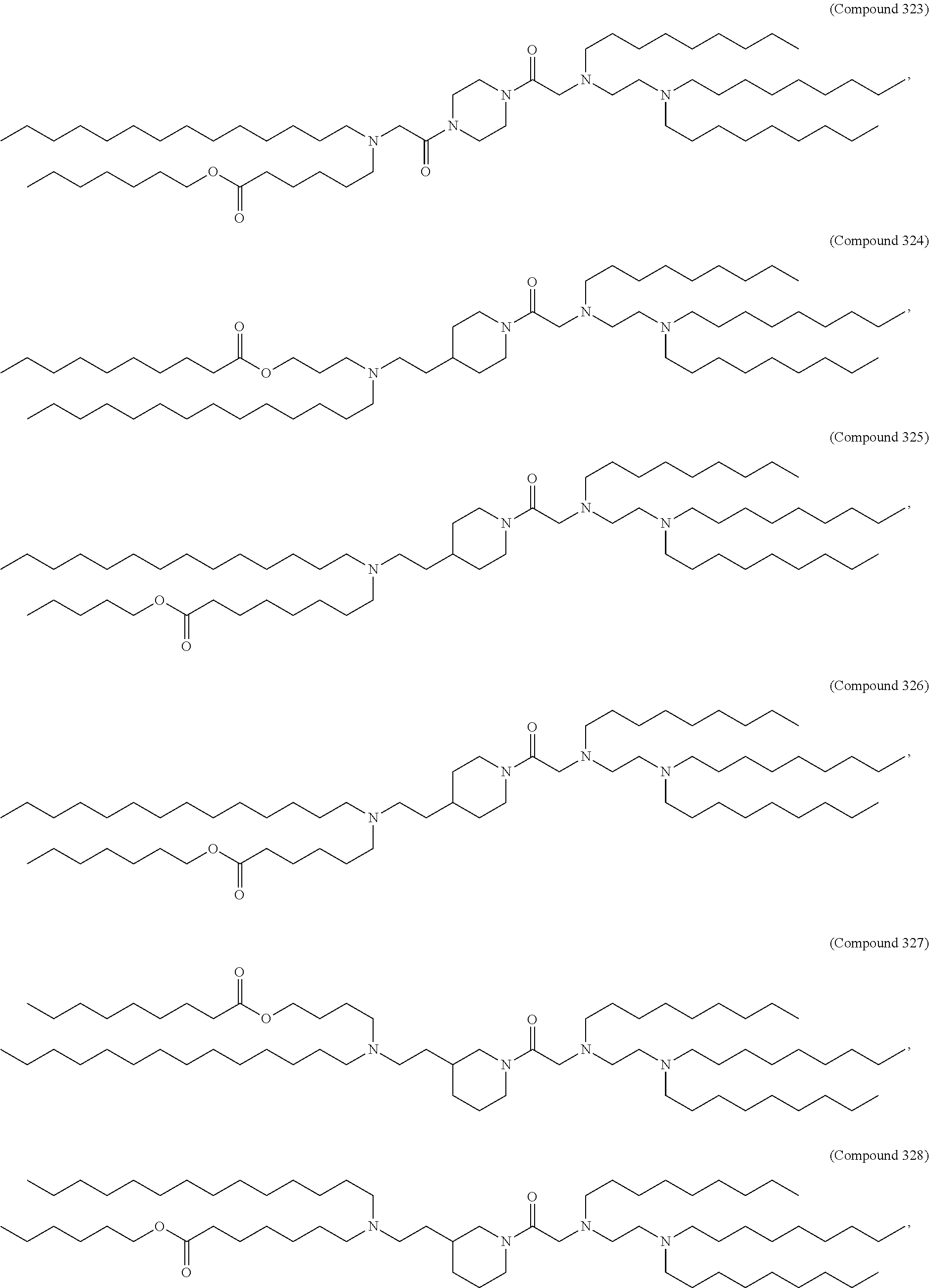

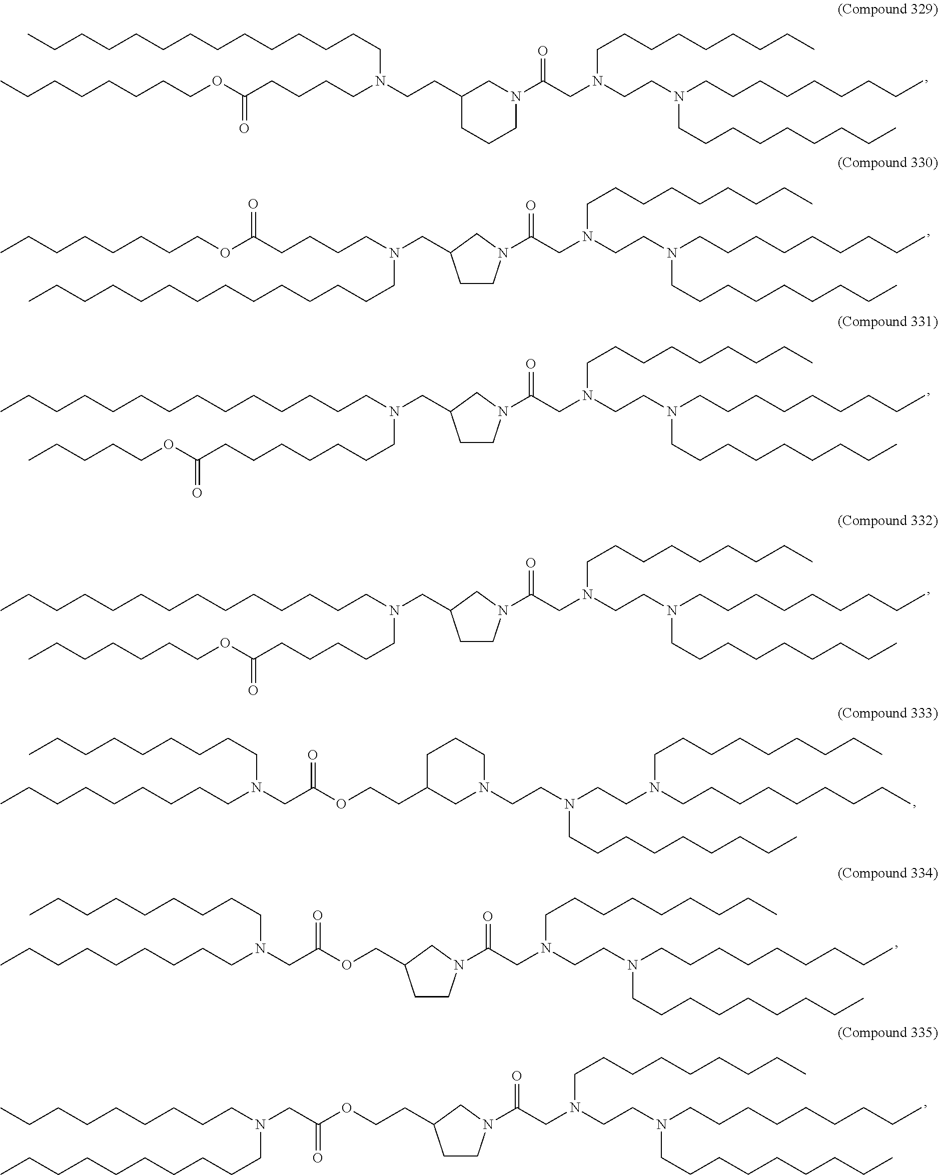

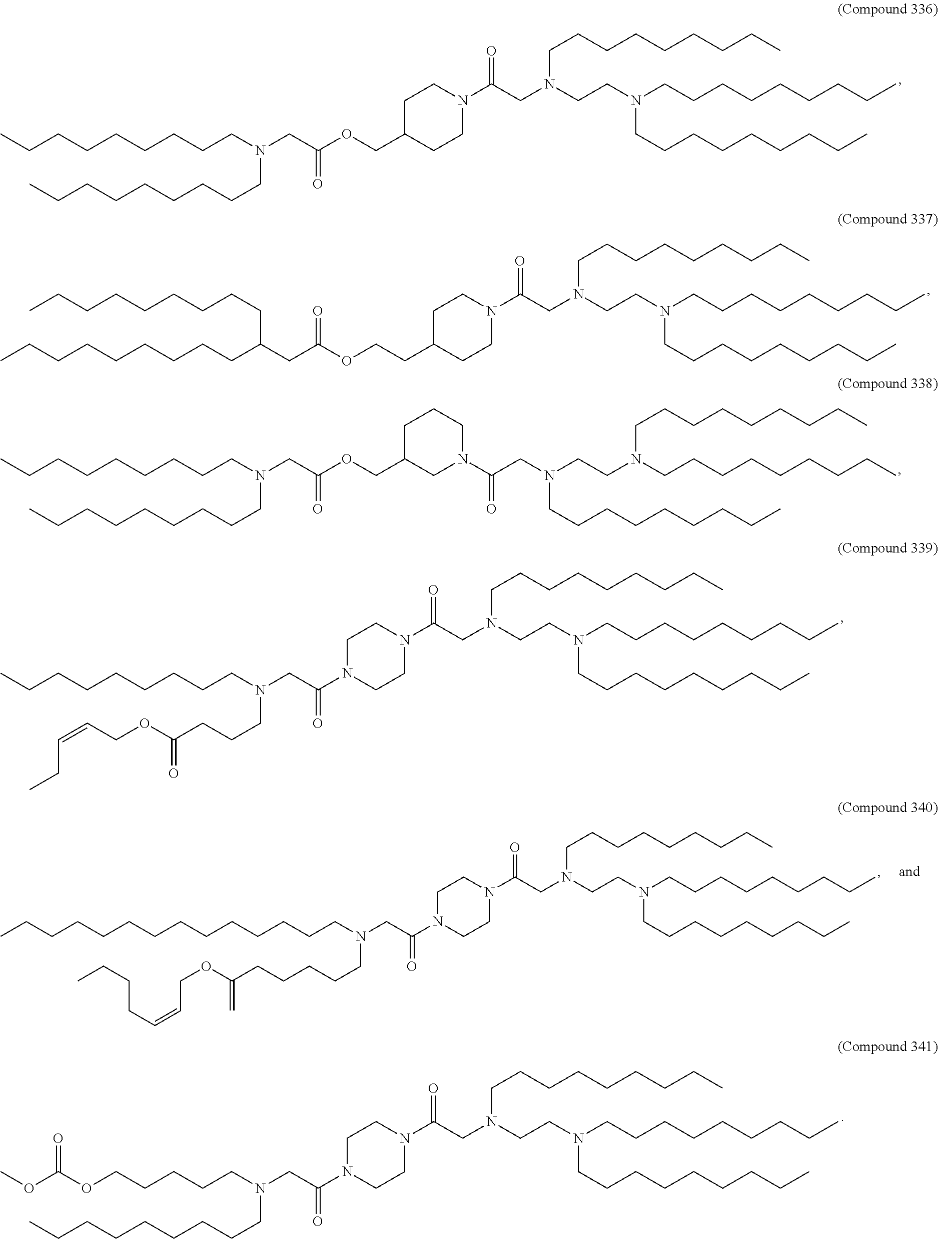

In some embodiments, the delivery agent comprises Compound 236.

In some embodiments, the delivery agent comprises a compound having the formula (IV)

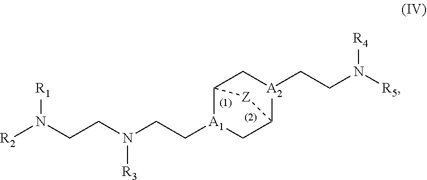

or salts or stereoisomer thereof, wherein

A₁ and A₂ are each independently selected from CH or N and at least one of A₁ and A₂ is N;

Z is CH₂ or absent wherein when Z is CH₂, the dashed lines (1) and (2) each represent a single bond; and when Z is absent, the dashed lines (1) and (2) are both absent;

R₁, R₂, R₃, R₄, and R₅ are independently selected from the group consisting of C₆₋₂₀ alkyl and C₆₋₂₀ alkenyl;

wherein when ring A is

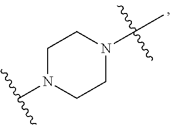 [SMILES: *N1CCN(*)CC1]

then

i) R₁, R₂, R₃, R₄, and R₅ are the same, wherein R₁ is not C₁₂ alkyl, C₁₈ alkyl, or C₁₈ alkenyl;

ii) only one of R₁, R₂, R₃, R₄, and R₅ is selected from C₆₋₂₀ alkenyl;

iii) at least one of R₁, R₂, R₃, R₄, and R₅ have a different number of carbon atoms than at least one other of R₁, R₂, R₃, R₄, and R₅;

iv) R₁, R₂, and R₃ are selected from C₆₋₂₀ alkenyl, and R₄ and R₅ are selected from C₆₋₂₀ alkyl; or

v) R₁, R₂, and R₃ are selected from C₆₋₂₀ alkyl, and R₄ and R₅ are selected from C₆₋₂₀ alkenyl.

In some embodiments, the compound is of formula (IVa):

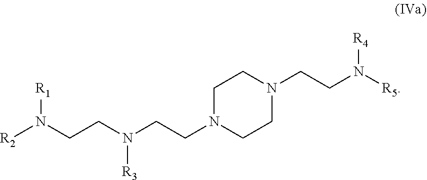

The compounds of Formula (IV) or (IVa) include one or more of the following features when applicable.

In some embodiments, Z is CH₂.

In some embodiments, Z is absent.

In some embodiments, at least one of A₁ and A₂ is N.

In some embodiments, each of A₁ and A₂ is N.

In some embodiments, each of A₁ and A₂ is CH.

In some embodiments, A₁ is N and A₂ is CH.

In some embodiments, A₁ is CH and A₂ is N.

In some embodiments, R₁, R₂, R₃, R₄, and R₅ are the same, and are not C₁₂ alkyl, C₁₈ alkyl, or C₁₈ alkenyl. In some embodiments, R₁, R₂, R₃, R₄, and R₅ are the same and are C₉ alkyl or C₁₄ alkyl.

In some embodiments, only one of R₁, R₂, R₃, R₄, and R₅ is selected from C₆₋₂₀ alkenyl. In certain such embodiments, R₁, R₂, R₃, R₄, and R₅ have the same number of carbon atoms. In some embodiments, R₄ is selected from C₅₋₂₀ alkenyl. For example, R₄ may be C₁₂ alkenyl or C₁₈ alkenyl.

In some embodiments, at least one of R₁, R₂, R₃, R₄, and R₅ have a different number of carbon atoms than at least one other of R₁, R₂, R₃, R₄, and R₅.

In certain embodiments, R₁, R₂, and R₃ are selected from C₆₋₂₀ alkenyl, and R₄ and R₅ are selected from C₆₋₂₀ alkyl. In other embodiments, R₁, R₂, and R₃ are selected from C₆₋₂₀ alkyl, and R₄ and R₅ are selected from C₆₋₂₀ alkenyl. In some embodiments, R₁, R₂, and R₃ have the same number of carbon atoms, and/or R₄ and R₅ have the same number of carbon atoms. For example, R₁, R₂, and R₃, or R₄ and R₅, may have 6, 8, 9, 12, 14, or 18 carbon atoms. In some embodiments, R₁, R₂, and R₃, or R₄ and R₅, are C₁₈ alkenyl (e.g., linoleyl). In some embodiments, R₁, R₂, and R₃, or R₄ and R₅, are alkyl groups including 6, 8, 9, 12, or 14 carbon atoms.

In some embodiments, R₁ has a different number of carbon atoms than R₂, R₃, R₄, and R₈. In other embodiments, R₃ has a different number of carbon atoms than R₁, R₂, R₄, and R₅. In further embodiments, R₄ has a different number of carbon atoms than R₁, R₂, R₃, and R₅.

In some embodiments, the compound is selected from the group consisting of:

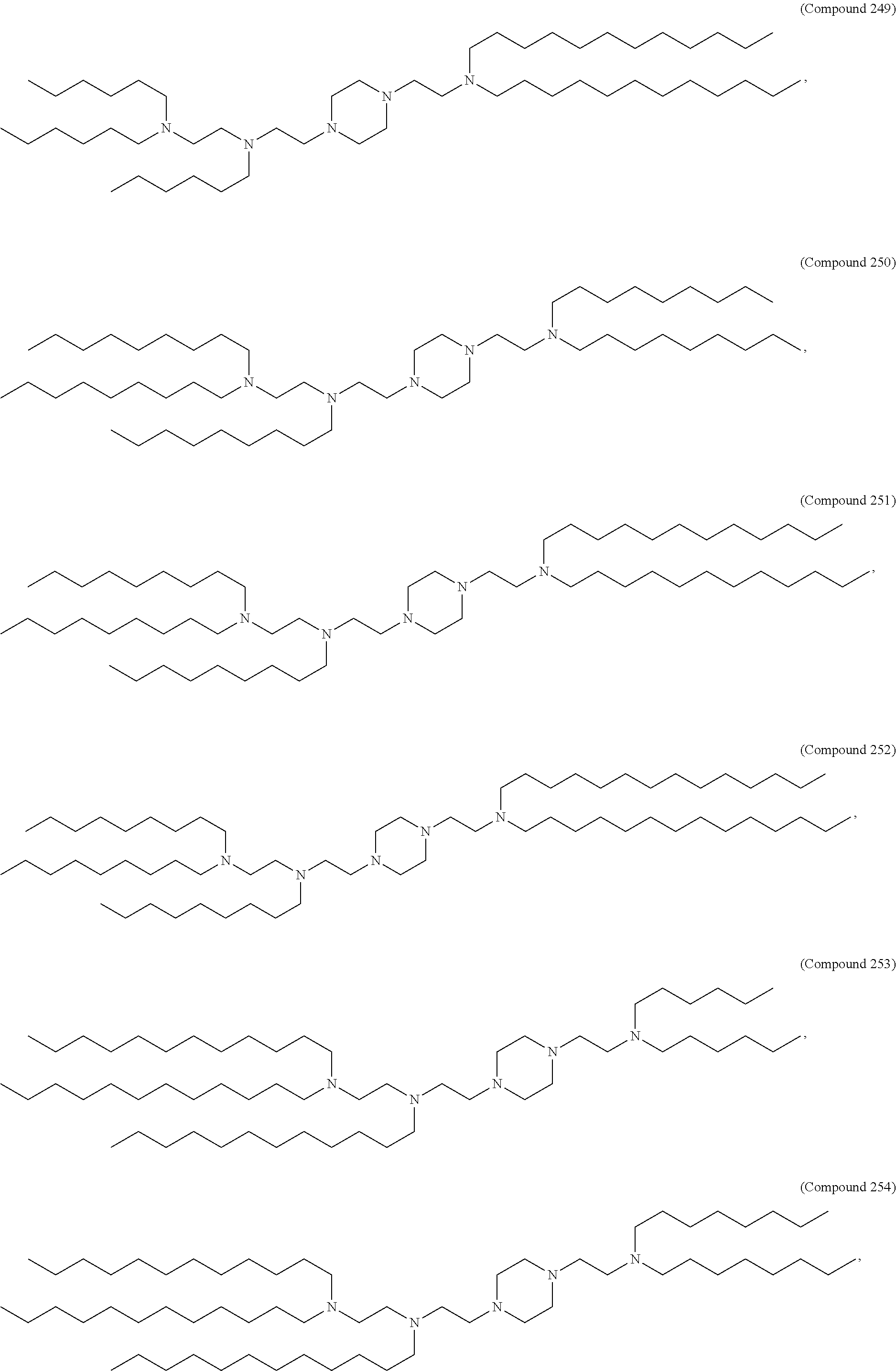

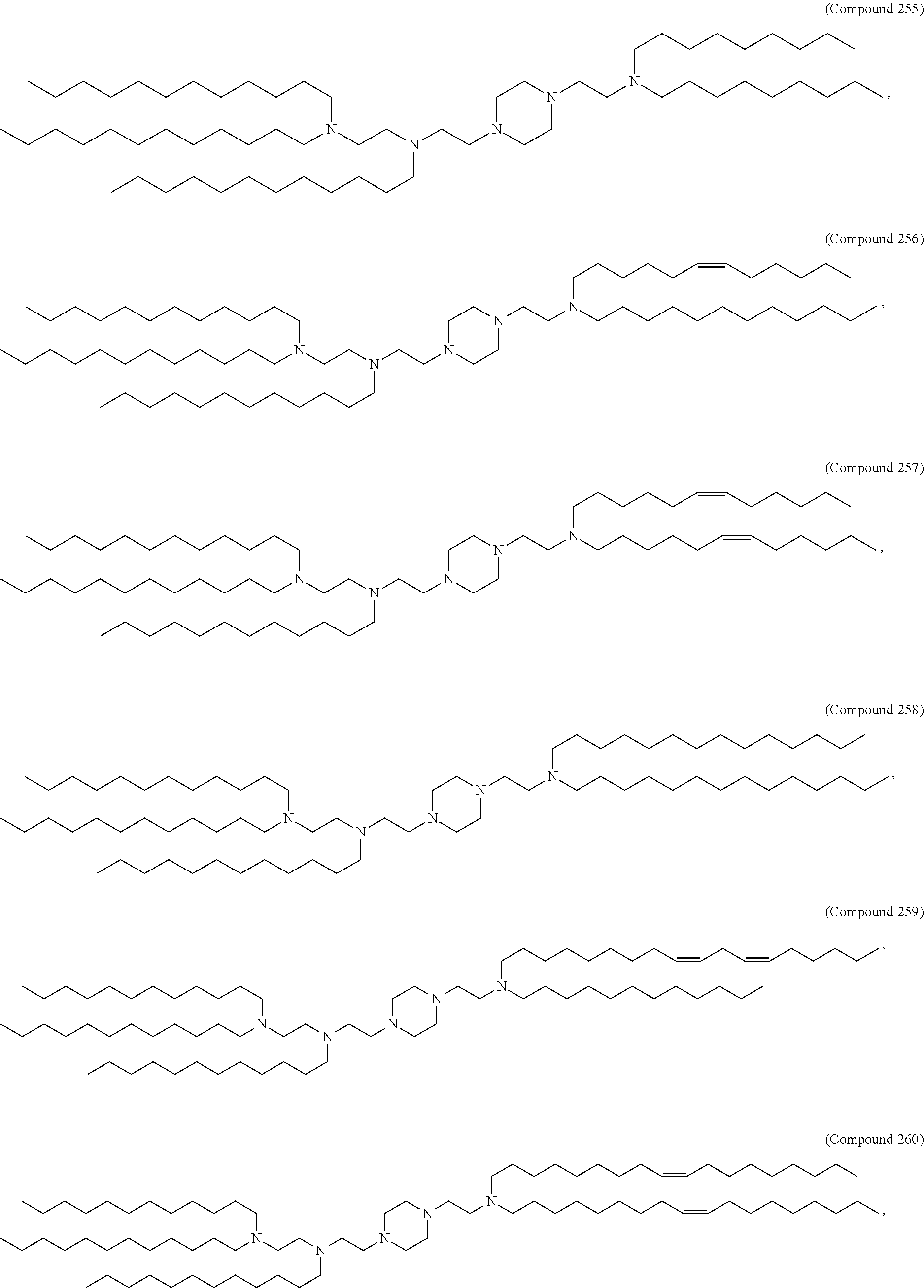

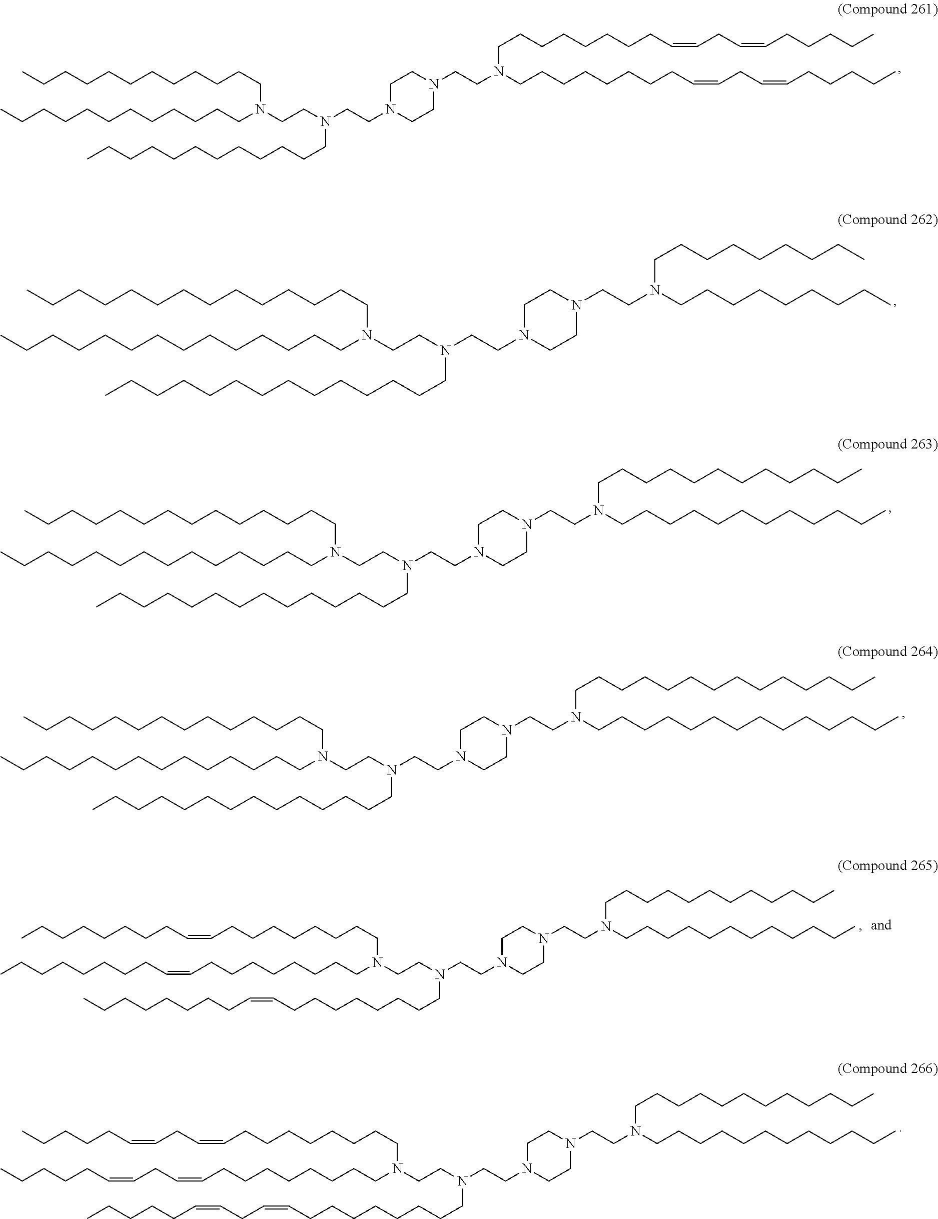

In other embodiments, the delivery agent comprises a compound having the formula (V)

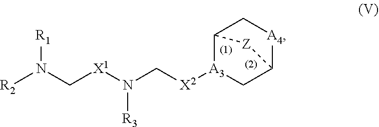

or salts or stereoisomers thereof, in which

A₃ is CH or N;

A₄ is CH₂ or NH; and at least one of A₃ and A₄ is N or NH;

Z is CH₂ or absent wherein when Z is CH₂, the dashed lines (1) and (2) each represent a single bond; and when Z is absent, the dashed lines (1) and (2) are both absent;

R₁, R₂, and R₃ are independently selected from the group consisting of C₅₋₂₀ alkyl, C₅₋₂₀ alkenyl, —R″MR′, —R*YR″, —YR″, and —R*OR″;

each M is independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

X¹ and X² are independently selected from the group consisting of —CH₂—, —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;

each Y is independently a C₃₋₆ carbocycle;

each R* is independently selected from the group consisting of C₁₋₁₂ alkyl and C₂₋₁₂ alkenyl;

each R is independently selected from the group consisting of C₁₋₃ alkyl and a C₃₋₆ carbocycle;

each R′ is independently selected from the group consisting of C₁₋₁₂ alkyl, C₂₋₁₂ alkenyl, and H; and

each R″ is independently selected from the group consisting of C₃₋₁₂ alkyl and C₃₋₁₂ alkenyl.

In some embodiments, the compound is of formula (Va):

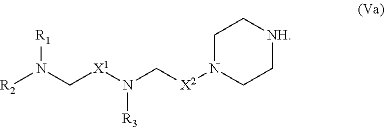

The compounds of Formula (V) or (Va) include one or more of the following features when applicable.

In some embodiments, Z is CH₂.

In some embodiments, Z is absent.

In some embodiments, at least one of A₃ and A₄ is N or NH.

In some embodiments, A₃ is N and A₄ is NH.

In some embodiments, A₃ is N and A₄ is CH₂.

In some embodiments, A₃ is CH and A₄ is NH.

In some embodiments, at least one of X¹ and X² is not —CH₂—. For example, in certain embodiments, X¹ is not —CH₂—. In some embodiments, at least one of X¹ and X² is —C(O)—.

In some embodiments, X² is —C(O)—, —C(O)O—, —OC(O)—, —C(O)—CH₂—, —CH₂—C(O)—, —C(O)O—CH₂—, —OC(O)—CH₂—, —CH₂—C(O)O—, or —CH₂—OC(O)—.

In some embodiments, R₁, R₂, and R₃ are independently selected from the group consisting of C₅₋₂₀ alkyl and C₅₋₂₀ alkenyl. In some embodiments, R₁, R₂, and R₃ are the same. In certain embodiments, R₁, R₂, and R₃ are C₆, C₉, C₁₂, or C₁₄ alkyl. In other embodiments, R₁, R₂, and R₃ are C₁₈ alkenyl. For example, R₁, R₂, and R₃ may be linoleyl.

In some embodiments, the compound is selected from the group consisting of:

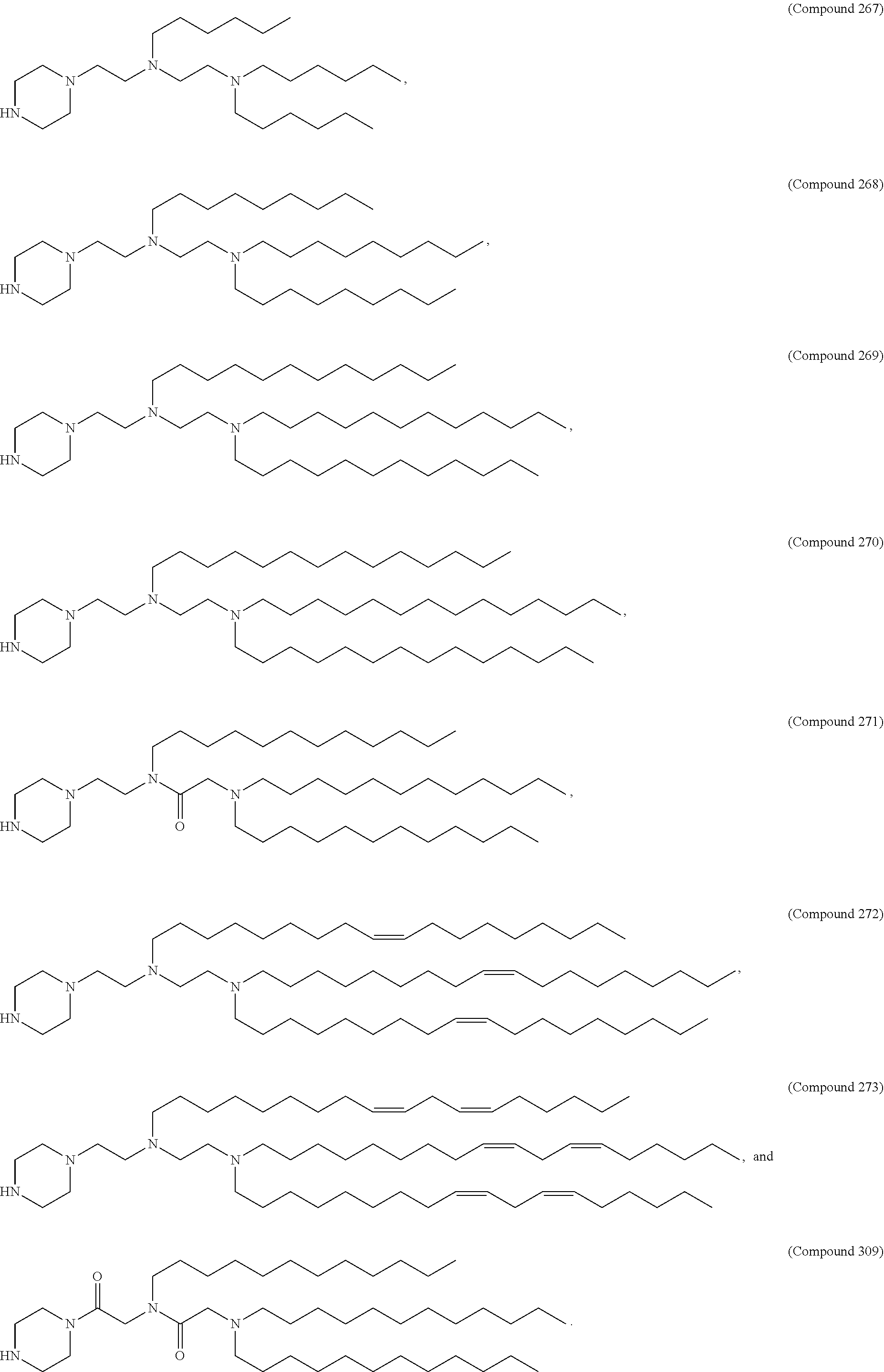

In other embodiments, the delivery agent comprises a compound having the formula (VI):

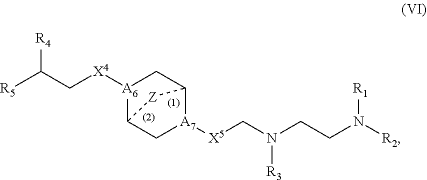

or salts or stereoisomers thereof, in which

A₆ and A₇ are each independently selected from CH or N, wherein at least one of A₆ and A₇ is N;

Z is CH₂ or absent wherein when Z is CH₂, the dashed lines (1) and (2) each represent a single bond; and when Z is absent, the dashed lines (1) and (2) are both absent;

X⁴ and X⁵ are independently selected from the group consisting of —CH₂—, —CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;

R₁, R₂, R₃, R₄, and R₅ each are independently selected from the group consisting of C₅₋₂₀ alkyl, C₅₋₂₀ alkenyl, —R″MR′, —R*YR″, —YR″, and —R*OR″;

each M is independently selected from the group consisting of —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—an aryl group, and a heteroaryl group;

each Y is independently a C₃₋₆ carbocycle;

each R* is independently selected from the group consisting of C₁₋₁₂ alkyl and C₂₋₁₂ alkenyl;

each R is independently selected from the group consisting of C₁₋₃ alkyl and a C₃₋₆ carbocycle;

each R′ is independently selected from the group consisting of C₁₋₁₂ alkyl, C₂₋₁₂ alkenyl, and H; and

each R″ is independently selected from the group consisting of C₃₋₁₂ alkyl and C₃₋₁₂ alkenyl.

In some embodiments, R₁, R₂, R₃, R₄, and R₅ each are independently selected from the group consisting of C₆₋₂₀ alkyl and C₆₋₂₀ alkenyl.

In some embodiments, R₁ and R₂ are the same. In certain embodiments, R₁, R₂, and R₃ are the same. In some embodiments, R₄ and R₅ are the same. In certain embodiments, R₁, R₂, R₃, R₄, and R₅ are the same.

In some embodiments, at least one of R₁, R₂, R₃, R₄, and R₅ is C₉₋₁₂ alkyl. In certain embodiments, each of R₁, R₂, R₃, R₄, and R₅ independently is C₉, C₁₂ or C₁₄ alkyl. In certain embodiments, each of R₁, R₂, R₃, R₄, and R₅ is C₉ alkyl.

In some embodiments, A₆ is N and A₇ is N. In some embodiments, A₆ is CH and A₇ is N.

In some embodiments, X⁴ is-CH₂- and X⁵ is —C(O)—. In some embodiments, X⁴ and X⁵ are —C(O)—.

In some embodiments, when A₆ is N and A₇ is N, at least one of X⁴ and X⁵ is not —CH₂—, e.g., at least one of X⁴ and X⁵ is —C(O)—. In some embodiments, when A₆ is N and A₇ is N, at least one of R₁, R₂, R₃, R₄, and R₅ is —R″MR′.

In some embodiments, at least one of R₁, R₂, R₃, R₄, and R₅ is not —R″MR′.

In some embodiments, the compound is

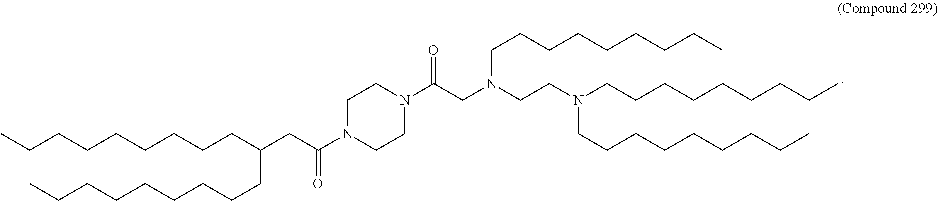 [SMILES: CCCCCCCCCC(CCCCCCCCC)CC(=O)N1CCN(C(=O)CN(CCCCCCCCC)CCN(CCCCCCCCC)CCCCCCCCC)CC1]

In other embodiments, the delivery agent comprises a compound having the formula:

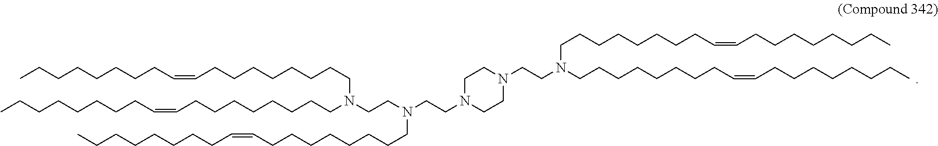 [SMILES: CCCCCCCC/C=C\CCCCCCCCN(CCCCCCCC/C=C\CCCCCCCC)CCN(CCCCCCCC/C=C\CCCCCCCC)CCN1CCN(CCN(CCCCCCCC/C=C\CCCCCCCC)CCCCCCCC/C=C\CCCCCCCC)CC1]

Amine moieties of the lipid compounds disclosed herein can be protonated under certain conditions. For example, the central amine moiety of a lipid according to formula (I) is typically protonated (i.e., positively charged) at a pH below the pKa of the amino moiety and is substantially not charged at a pH above the pKa. Such lipids can be referred to ionizable amino lipids.

In one specific embodiment, the ionizable amino lipid is Compound 18. In another embodiment, the ionizable amino lipid is Compound 236.

In some embodiments, the amount the ionizable amino lipid, e.g., compound of formula (I) ranges from about 1 mol % to 99 mol % in the lipid composition.

In one embodiment, the amount of the ionizable amino lipid, e.g., compound of formula (I) is at least about 1, 2, 3, 4, 5, 6, 7, 8, 9, 10, 11, 12, 13, 14, 15, 16, 17, 18, 19, 20, 5 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 mol % in the lipid composition.

In one embodiment, the amount of the ionizable amino lipid, e.g., the compound of formula (I) ranges from about 30 mol % to about 70 mol %, from about 35 mol % to about 65 mol %, from about 40 mol % to about 60 mol %, and from about 45 mol % to about 55 mol % in the lipid composition.

In one specific embodiment, the amount of the ionizable amino lipid, e.g., compound of formula (I) is about 50 mol % in the lipid composition.

In addition to the ionizable amino lipid disclosed herein, e.g., compound of formula (I), the lipid composition of the pharmaceutical compositions disclosed herein can comprise additional components such as phospholipids, structural lipids, PEG-lipids, and any combination thereof.

### Phospholipids

The lipid composition of the pharmaceutical composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

Examples of phospholipids include, but are not limited to, the following:

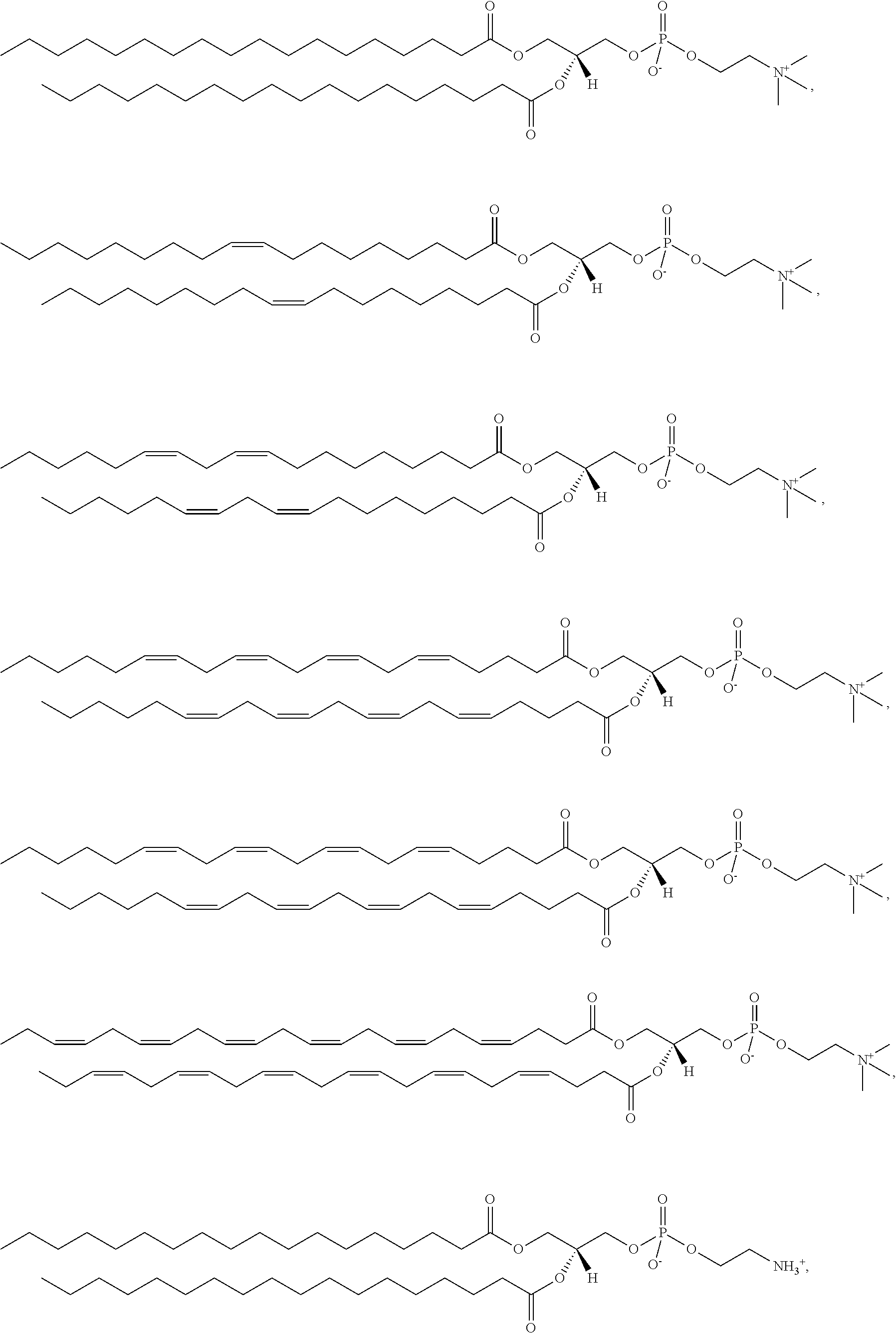

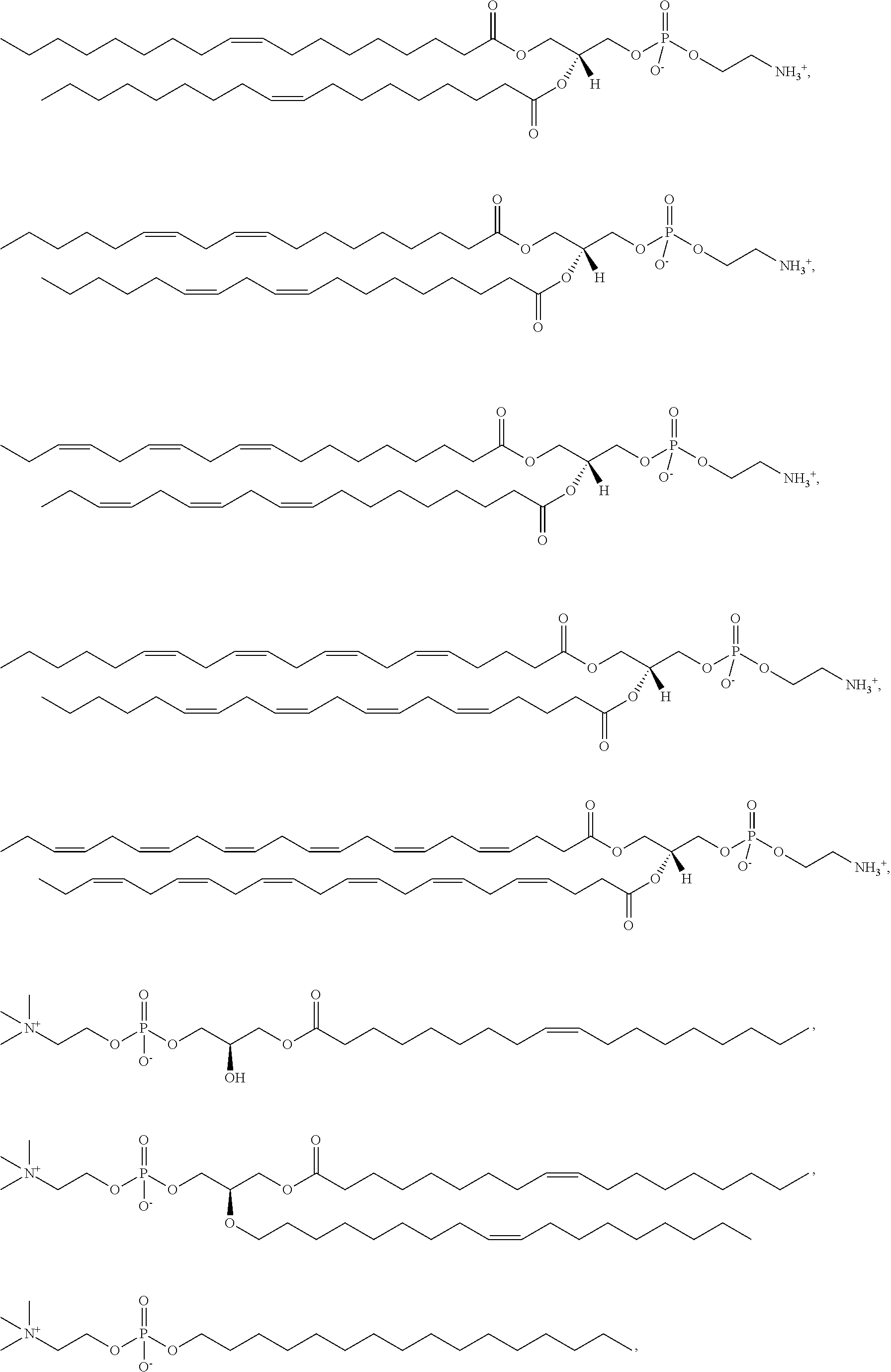

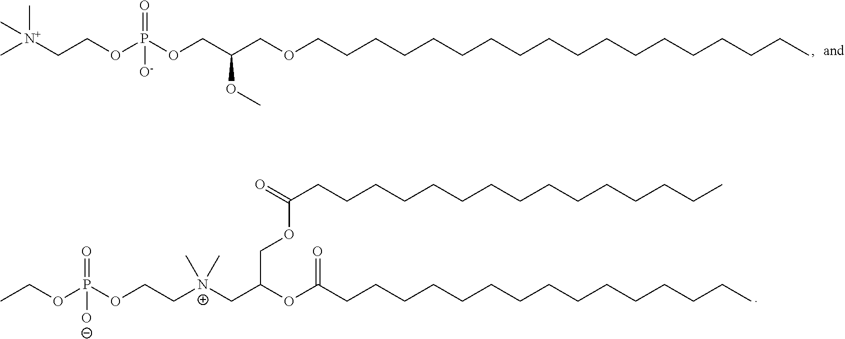

In certain embodiments, a phospholipid useful or potentially useful in the present invention is an analog or variant of DSPC. In certain embodiments, a phospholipid useful or potentially useful in the present invention is a compound of Formula (IX):

or a salt thereof, wherein:

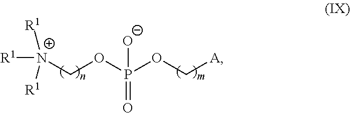

each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;

n is 1, 2, 3, 4, 5, 6, 7, 8, 9, or 10;

m is 0, 1, 2, 3, 4, 5, 6, 7, 8, 9, or 10;

A is of the formula:

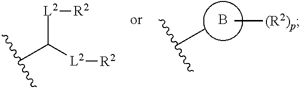

each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, -NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2;

provided that the compound is not of the formula:

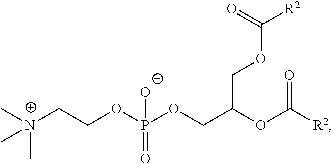

wherein each instance of R² is independently unsubstituted alkyl, unsubstituted alkenyl, or unsubstituted alkynyl.

### i) Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IX), at least one of R¹ is not methyl. In certain embodiments, at least one of R¹ is not hydrogen or methyl. In certain embodiments, the compound of Formula (IX) is of one of the following formulae:

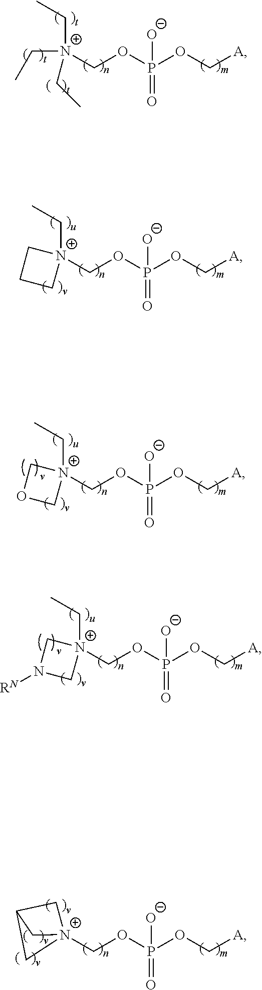

or a salt thereof, wherein:

each t is independently 1, 2, 3, 4, 5, 6, 7, 8, 9, or 10;

each u is independently 0, 1, 2, 3, 4, 5, 6, 7, 8, 9, or 10; and

each v is independently 1, 2, or 3.

In certain embodiments, the compound of Formula (IX) is of one of the following formulae:

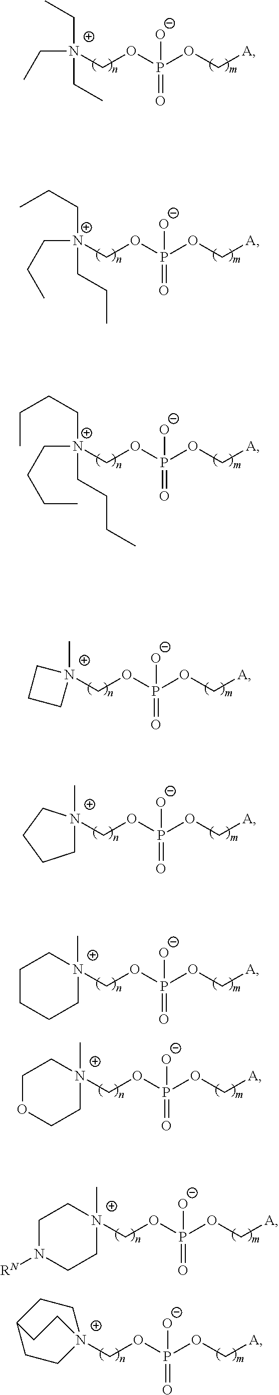

or a salt thereof.

In certain embodiments, a compound of Formula (IX) is one of the following:

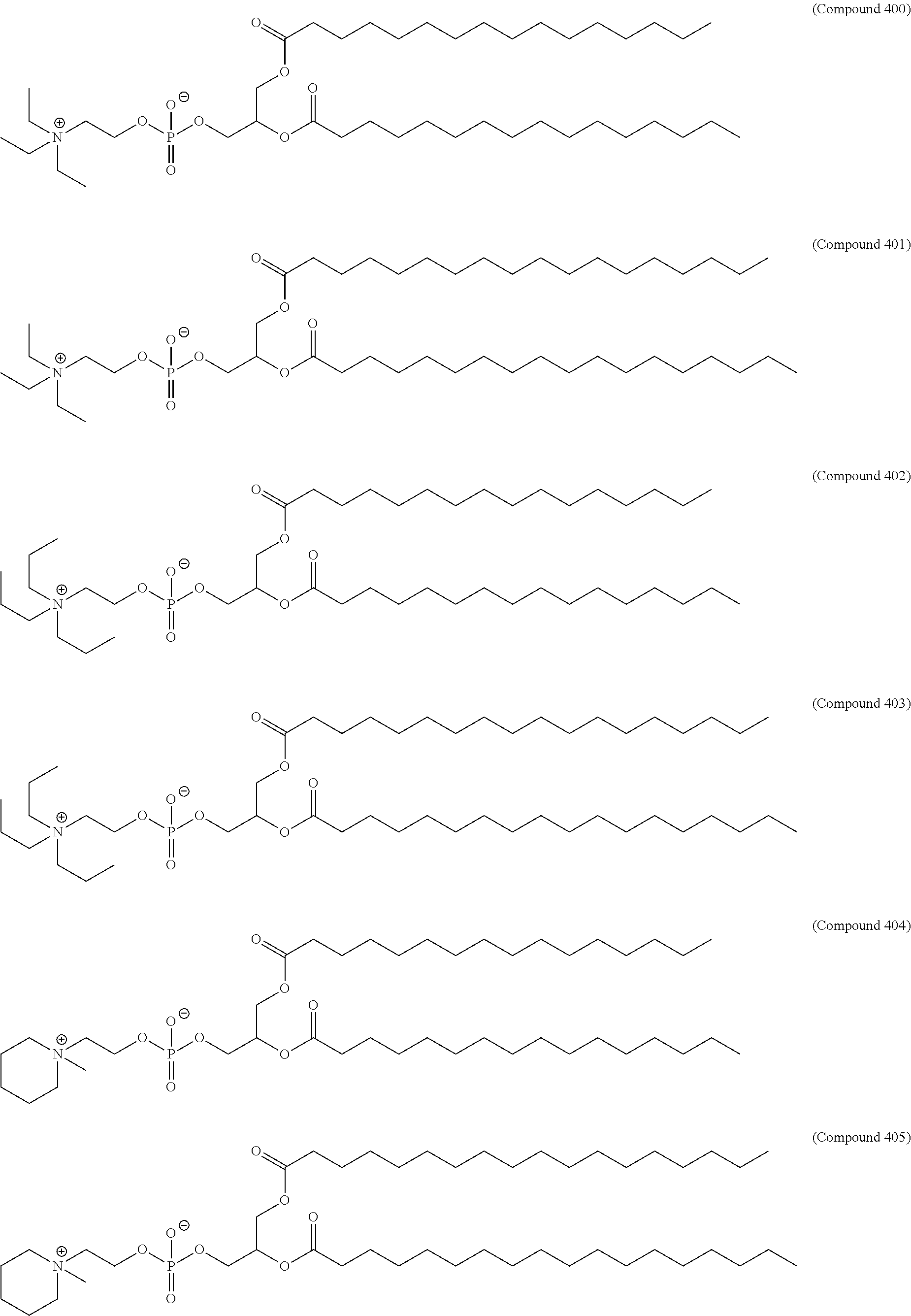

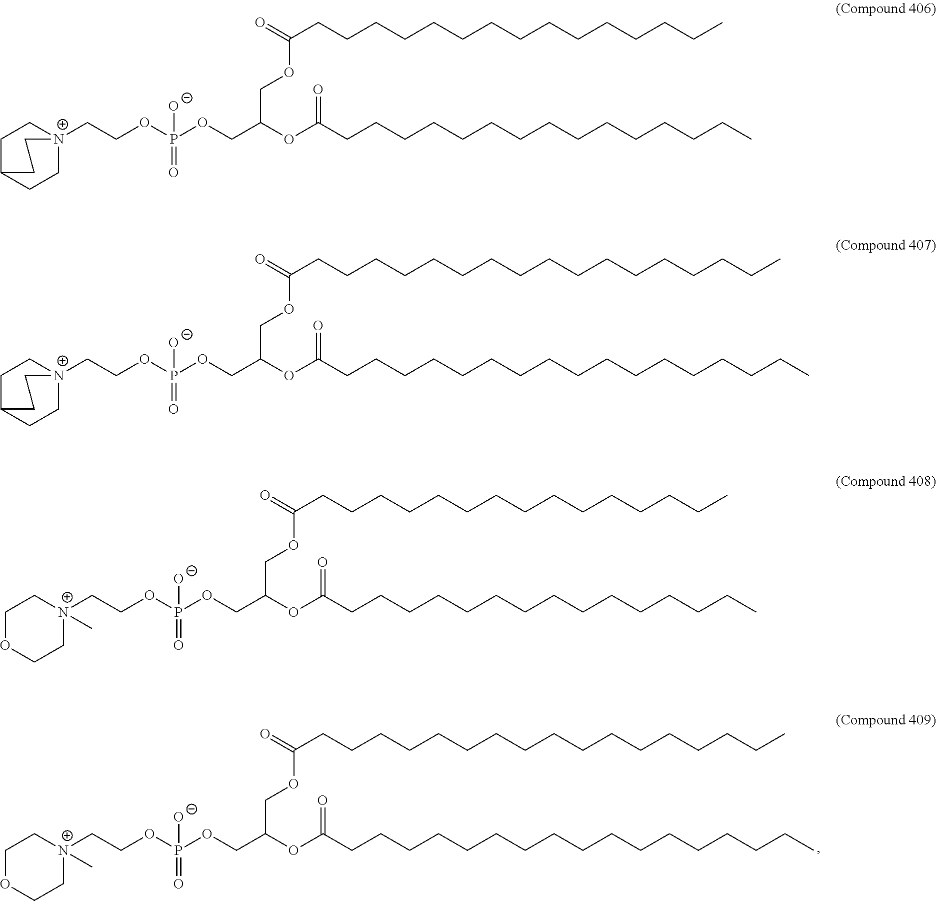 [SMILES: CCCCCCCCCCCCCCCC(=O)OCC(COP(=O)([O-])OCC[N+]12CCC(CC1)CC2)OC(=O)CCCCCCCCCCCCCCC ; CCCCCCCCCCCCCCCCCC(=O)OCC(COP(=O)([O-])OCC[N+]12CCC(CC1)CC2)OC(=O)CCCCCCCCCCCCCCCCC ; CCCCCCCCCCCCCCCC(=O)OCC(COP(=O)([O-])OCC[N+]1(C)CCOCC1)OC(=O)CCCCCCCCCCCCCCC ; CCCCCCCCCCCCCCCCCC(=O)OCC(COP(=O)([O-])OCC[N+]1(C)CCOCC1)OC(=O)CCCCCCCCCCCCCCCCC]

or a salt thereof.

In certain embodiments, a compound of Formula (IX) is of Formula (IX-a):

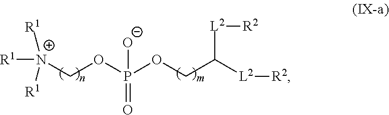

or a salt thereof.

In certain embodiments, phospholipids useful or potentially useful in the present invention comprise a modified core. In certain embodiments, a phospholipid with a modified core described herein is DSPC, or analog thereof, with a modified core structure. For example, in certain embodiments of Formula (IX-a), group A is not of the following formula:

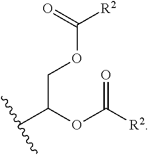

In certain embodiments, the compound of Formula (IX-a) is of one of the following formulae:

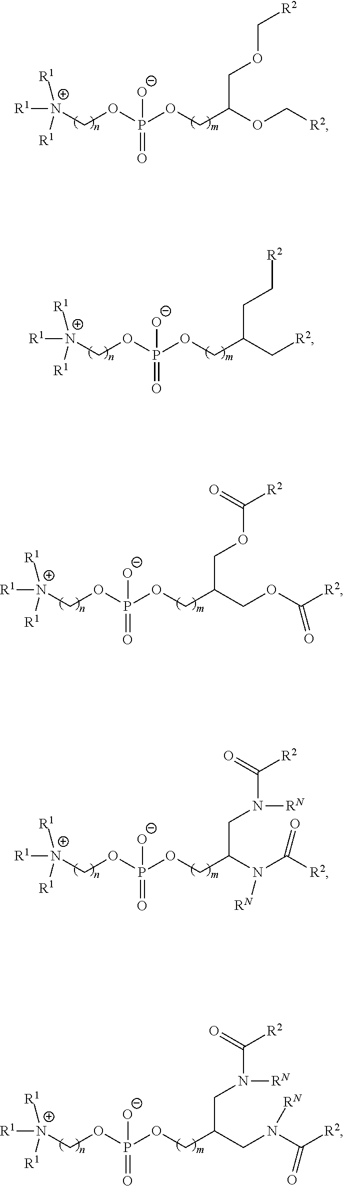

or a salt thereof

In certain embodiments, a compound of Formula (IX) is one of the following:

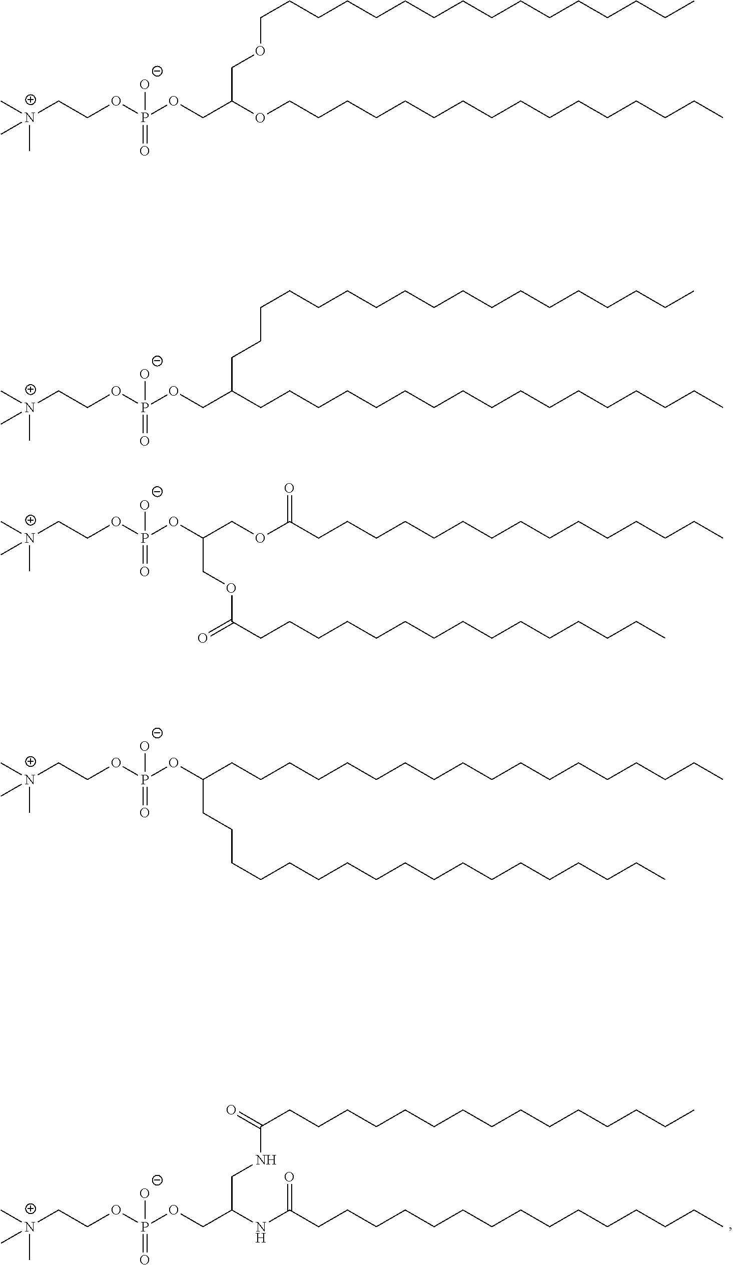

or salts thereof.

In certain embodiments, a phospholipid useful or potentially useful in the present invention comprises a cyclic moiety in place of the glyceride moiety. In certain embodiments, a phospholipid useful in the present invention is DSPC, or analog thereof, with a cyclic moiety in place of the glyceride moiety. In certain embodiments, the compound of Formula (IX) is of Formula (IX-b):

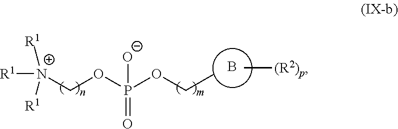

or a salt thereof.

In certain embodiments, the compound of Formula (IX-b) is of Formula (IX-b-1):

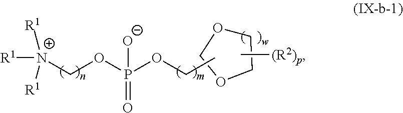

or a salt thereof, wherein:

w is 0, 1, 2, or 3.

In certain embodiments, the compound of Formula (IX-b) is of Formula (IX-b-2):

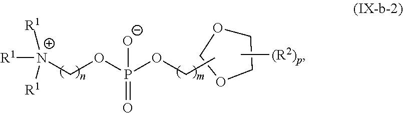

or a salt thereof.

In certain embodiments, the compound of Formula (IX-b) is of Formula (IX-b-3):

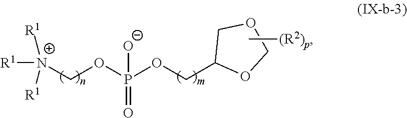

or a salt thereof.

In certain embodiments, the compound of Formula (IX-b) is of Formula (IX-b-4):

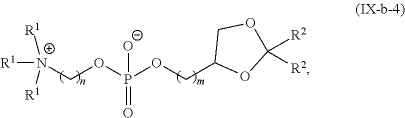

or a salt thereof.

In certain embodiments, the compound of Formula (IX-b) is one of the following:

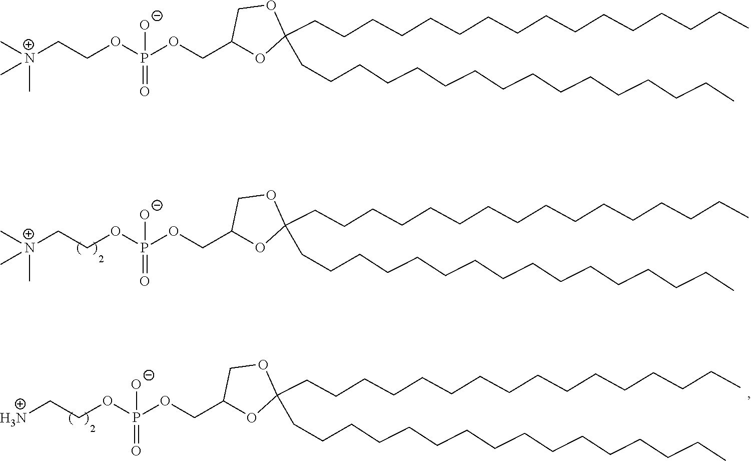

or salts thereof.

### (ii) Phospholipid Tail Modifications

In certain embodiments, a phospholipid useful or potentially useful in the present invention comprises a modified tail. In certain embodiments, a phospholipid useful or potentially useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (IX) is of Formula (IX-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In certain embodiments, the compound of Formula (IX) is of Formula (IX-c):

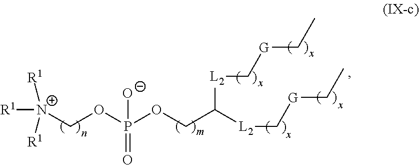

or a salt thereof, wherein:

each x is independently an integer between 0-30, inclusive; and

each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present invention.

In certain embodiments, the compound of Formula (IX-c) is of Formula (IX-c-1):

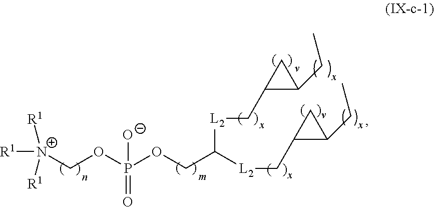

or salt thereof, wherein:

each instance of v is independently 1, 2, or 3.

In certain embodiments, the compound of Formula (IX-c) is of Formula (IX-c-2):

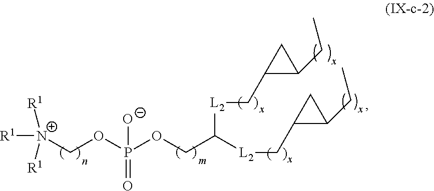

or a salt thereof.

In certain embodiments, the compound of Formula (IX-c) is of the following formula:

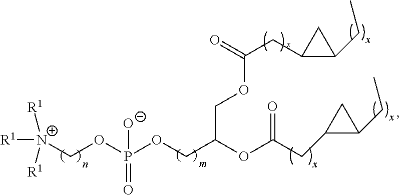

or a salt thereof.

In certain embodiments, the compound of Formula (IX-c) is the following:

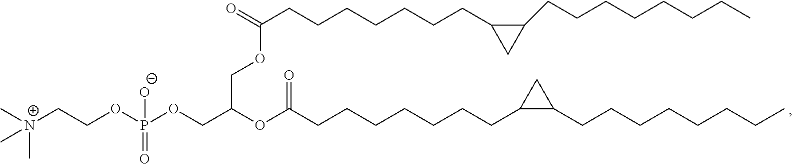 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (IX-c) is of Formula (IX-c-3):

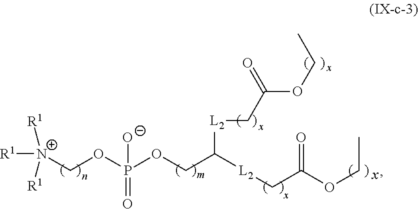

or a salt thereof.

In certain embodiments, the compound of Formula (IX-c) is of the following formulae:

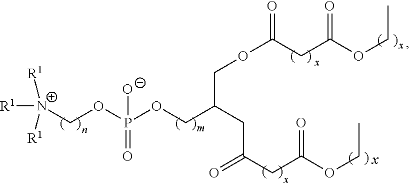

or a salt thereof.

In certain embodiments, the compound of Formula (IX-c) is the following:

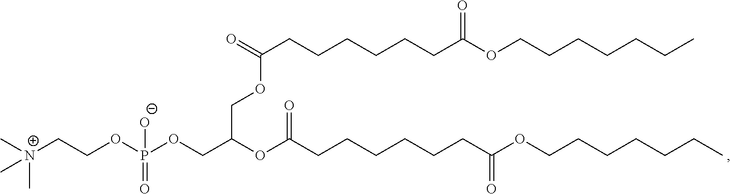 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (IX), wherein n is 1, 3, 4, 5, 6, 7, 8, 9, or 10. For example, in certain embodiments, a compound of Formula (IX) is of one of the following formulae:

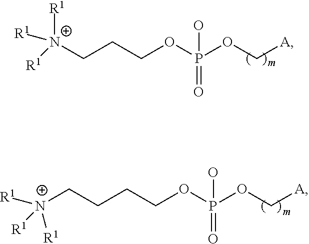

or a salt thereof.

In certain embodiments, a compound of Formula (IX) is one of the following:

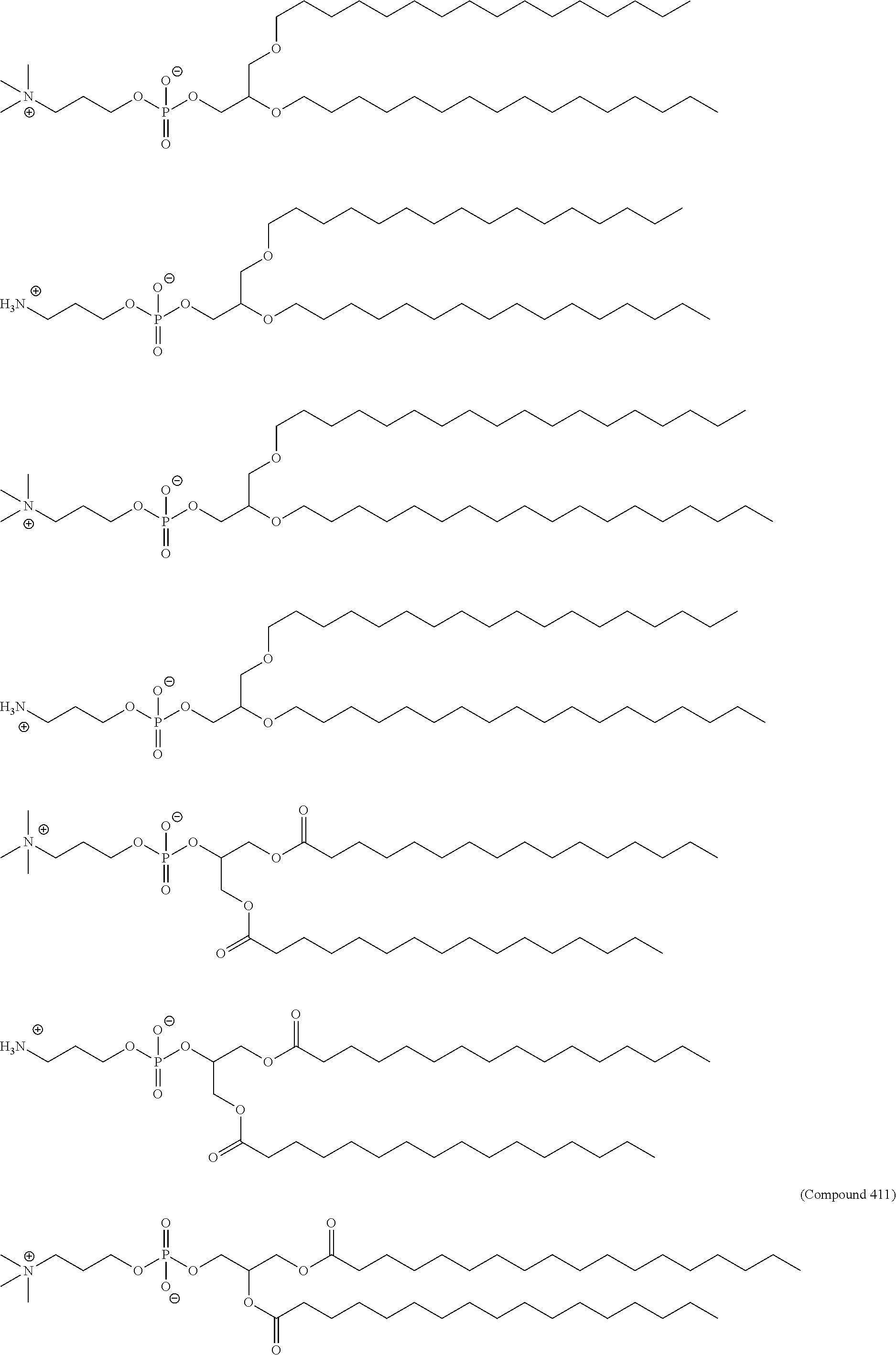

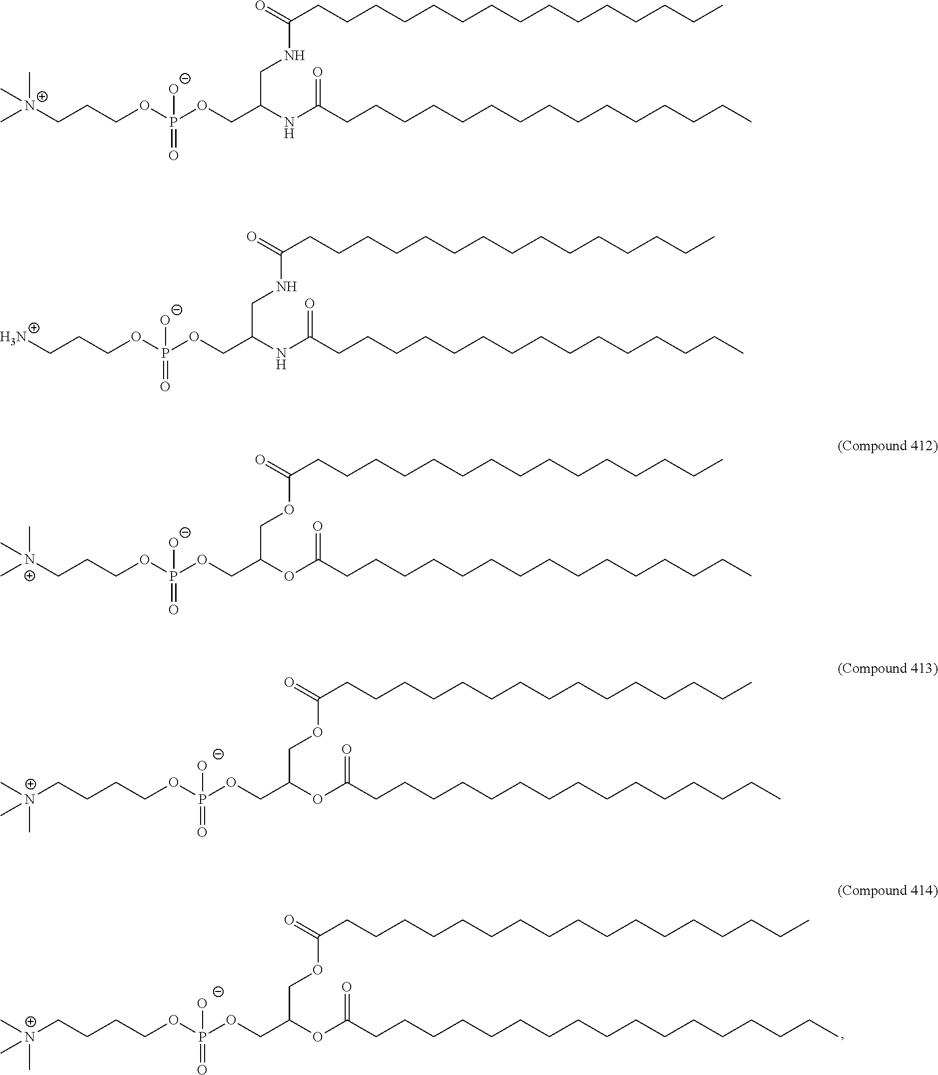

or salts thereof.

### Alternative Lipids

In certain embodiments, an alternative lipid is used in place of a phospholipid of the invention. Non-limiting examples of such alternative lipids include the following:

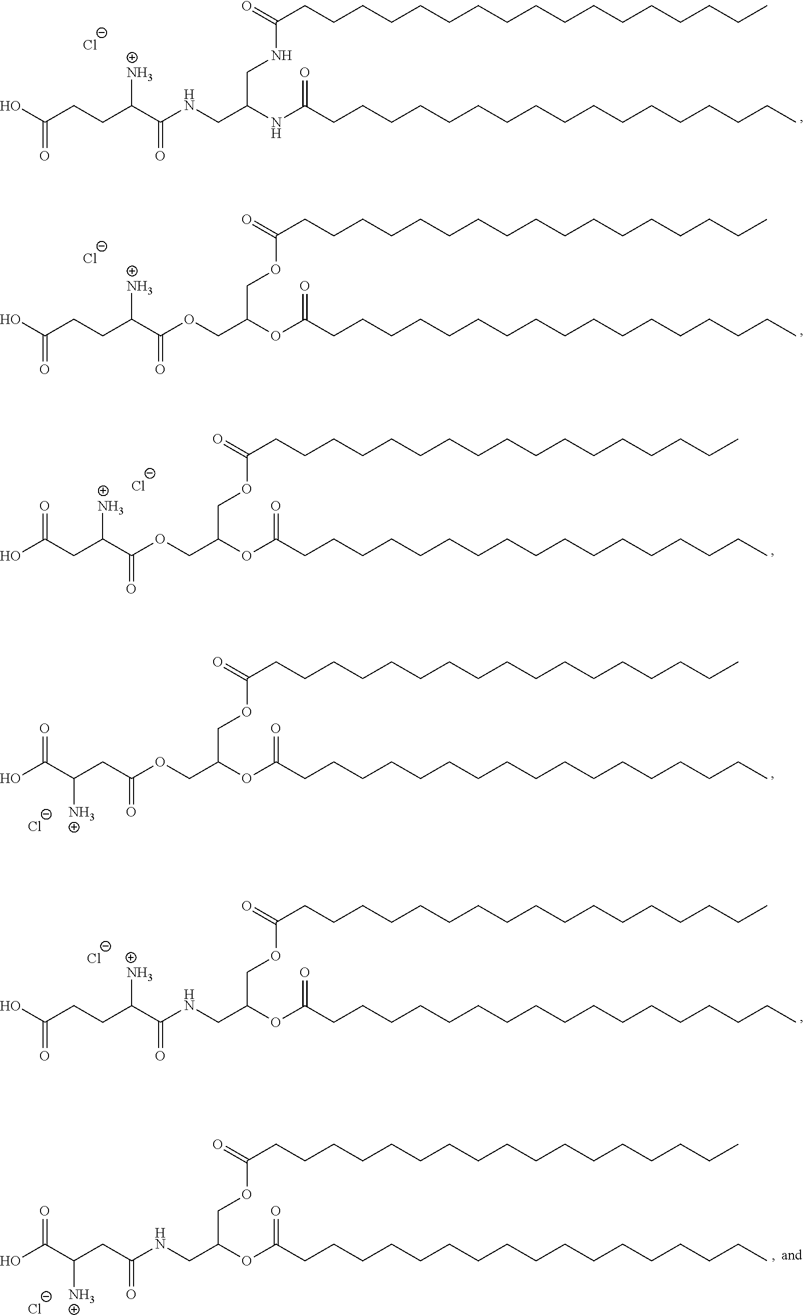

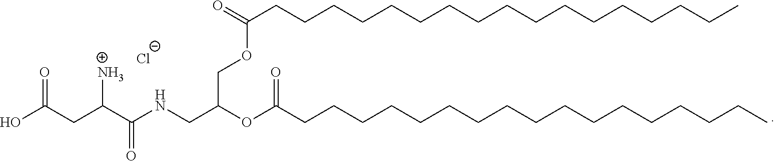 [SMILES: CCCCCCCCCCCCCCCCCC(=O)OCC(CNC(=O)C([NH3+])CC(=O)O)OC(=O)CCCCCCCCCCCCCCCCC ; [Cl-]]

### Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol. Examples of structural lipids include, but are not limited to, the following:

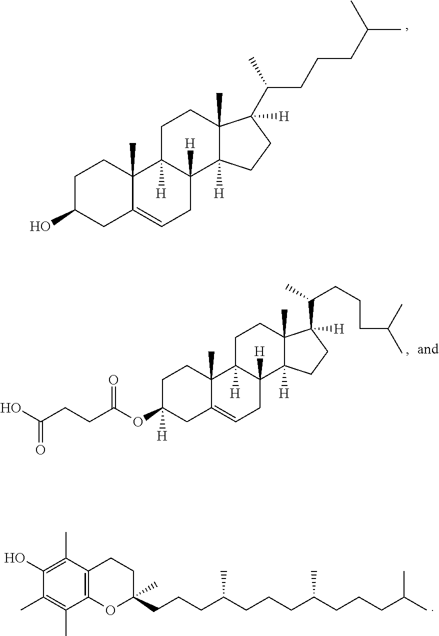

In one embodiment, the amount of the structural lipid (e.g., an sterol such as cholesterol) in the lipid composition of a pharmaceutical composition disclosed herein ranges from about 20 mol % to about 60 mol %, from about 25 mol % to about 55 mol %, from about 30 mol % to about 50 mol %, or from about 35 mol % to about 45 mol %.

In one embodiment, the amount of the structural lipid (e.g., an sterol such as cholesterol) in the lipid composition disclosed herein ranges from about 25 mol % to about 30 mol %, from about 30 mol % to about 35 mol %, or from about 35 mol % to about 40 mol %.

In one embodiment, the amount of the structural lipid (e.g., a sterol such as cholesterol) in the lipid composition disclosed herein is about 24 mol %, about 29 mol %, about 34 mol %, or about 39 mol %.

In some embodiments, the amount of the structural lipid (e.g., an sterol such as cholesterol) in the lipid composition disclosed herein is at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol %.

### Polyethylene Glycol (PEG)-Lipids

The lipid composition of a pharmaceutical composition disclosed herein can comprise one or more a polyethylene glycol (PEG) lipid.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanol amine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

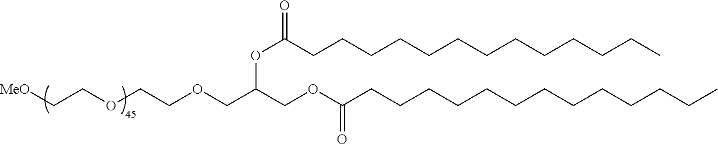

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (VII). Provided herein are compounds of Formula (VII):

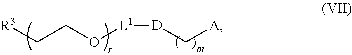

or salts thereof, wherein:

R³ is —OR^{O};

R^{O} is hydrogen, optionally substituted alkyl, or an oxygen protecting group;

r is an integer between 1 and 100, inclusive;

L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, —OC(O), OC(O)O, OC(O)N(R^{N}), NR^{N}C(O)O, or NR^{N}C(O)N(R^{N});

D is a moiety obtained by click chemistry or a moiety cleavable under physiological conditions;

m is 0, 1, 2, 3, 4, 5, 6, 7, 8, 9, or 10;

A is of the formula:

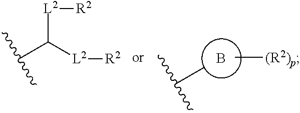

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O, C(O)S, SC(O), —C(═NR^{N}), C(═NR^{N})N(R^{N}), NR^{N}C(═NR^{N}), NR^{N}C(═NR)N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2.

In certain embodiments, the compound of Formula (VII) is a PEG-OH lipid (i.e., R³ is —OR^{O}, and R^{O} is hydrogen). In certain embodiments, the compound of Formula (VII) is of Formula (VII-OH):

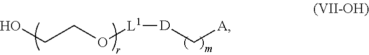

or a salt thereof.

In certain embodiments, D is a moiety obtained by click chemistry (e.g., triazole). In certain embodiments, the compound of Formula (VII) is of Formula (VII-a-1) or (VII-a-2):

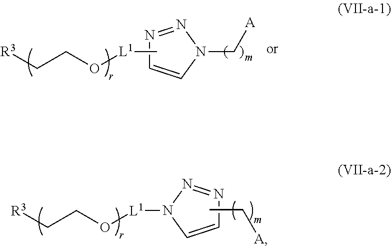

or a salt thereof.

In certain embodiments, the compound of Formula (VII) is of one of the following formulae:

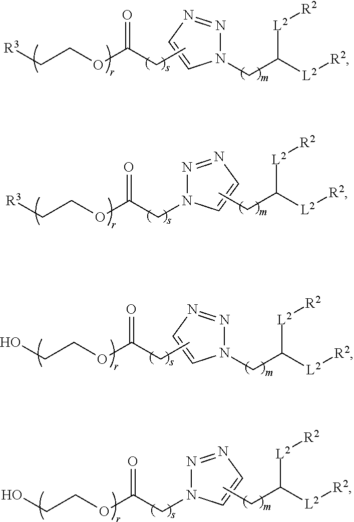

or a salt thereof, wherein

s is 0, 1, 2, 3, 4, 5, 6, 7, 8, 9, or 10.

In certain embodiments, the compound of Formula (VII) is of one of the following formulae:

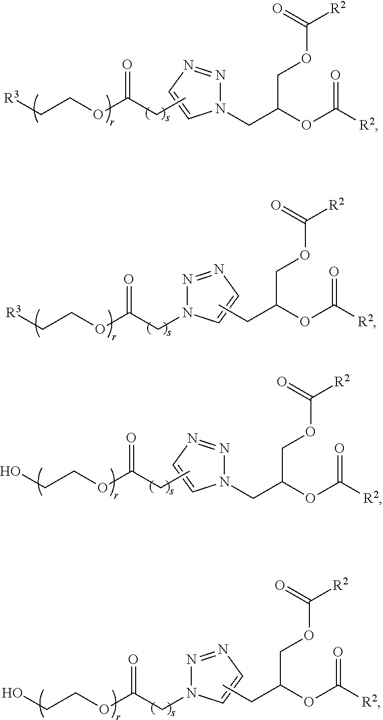

or a salt thereof.

In certain embodiments, a compound of Formula (VII) is of one of the following formulae:

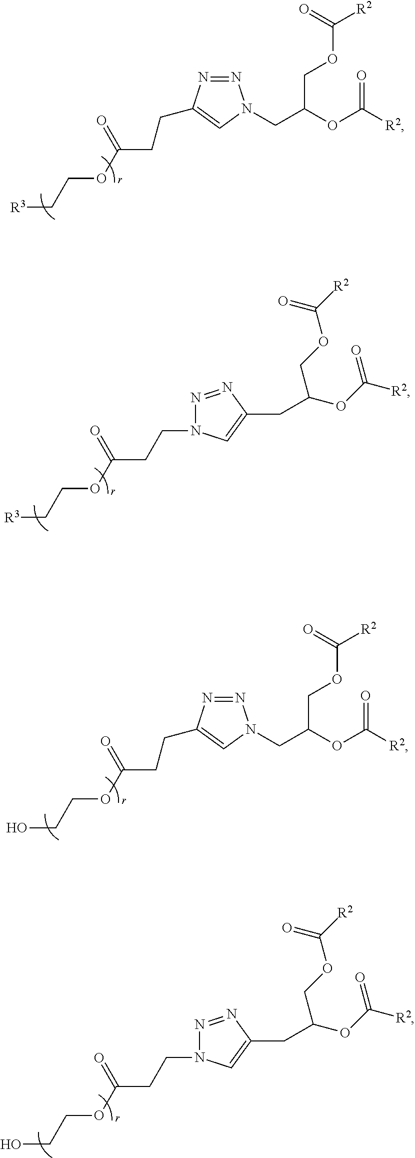

or a salt thereof.

In certain embodiments, a compound of Formula (VII) is of one of the following formulae:

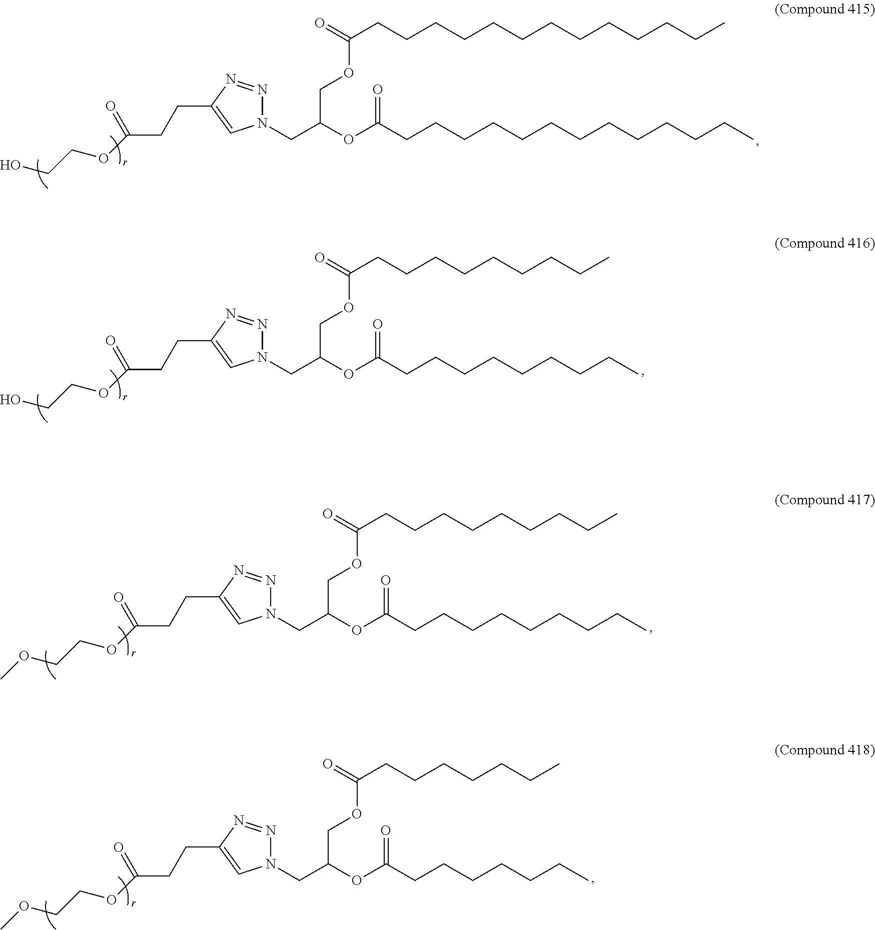

or a salt thereof.

In certain embodiments, D is a moiety cleavable under physiological conditions (e.g., ester, amide, carbonate, carbamate, urea). In certain embodiments, a compound of Formula (VII) is of Formula (VII-b-1) or (VII-b-2):

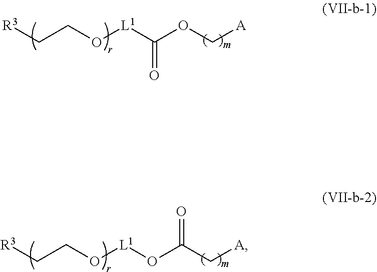

or a salt thereof

In certain embodiments, a compound of Formula (VII) is of Formula (VII-b-1-OH) or (VII-b-2-OH):

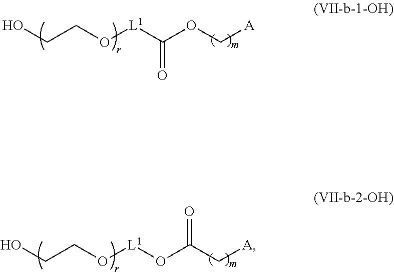

or a salt thereof.

In certain embodiments, the compound of Formula (VII) is of one of the following formulae:

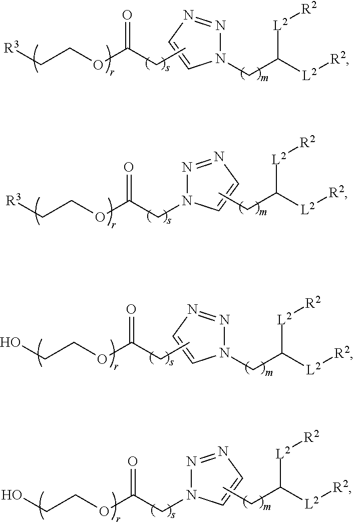

or a salt thereof.

In certain embodiments, a compound of Formula (VII) is of one of the following formulae:

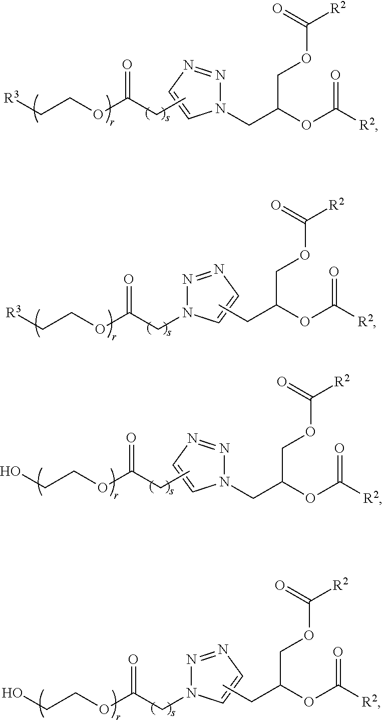

or a salt thereof.

In certain embodiments, a compound of Formula (VII) is of one of the following formulae:

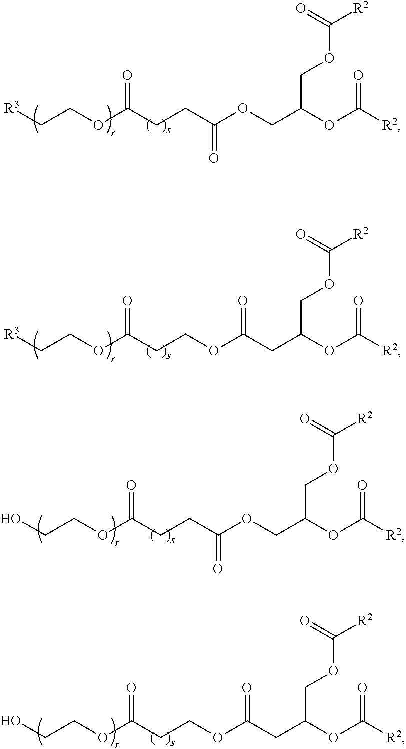

or a salt thereof.

In certain embodiments, a compound of Formula (VII) is of one of the following formulae:

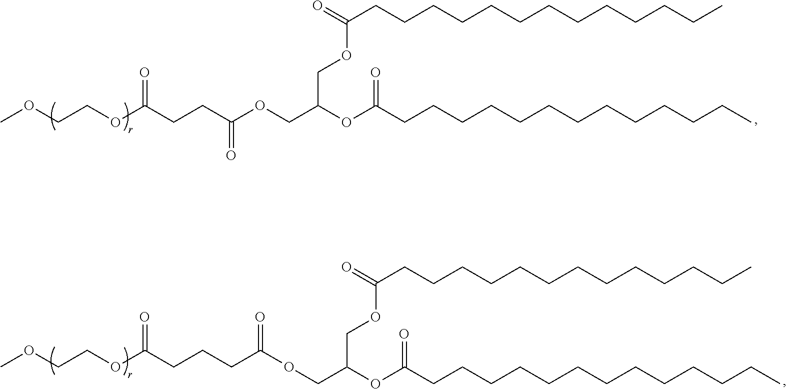

or salts thereof.

In certain embodiments, a PEG lipid useful in the present invention is a PEGylated fatty acid. In certain embodiments, a PEG lipid useful in the present invention is a compound of Formula (VIII). Provided herein are compounds of Formula (VIII):

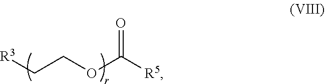

or a salts thereof, wherein:

R³ is —OR^{O};

R^{O} is hydrogen, optionally substituted alkyl or an oxygen protecting group;

r is an integer between 1 and 100, inclusive;

R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), —C(O)N(R^{N}), NR^{N}C(O), NR^{N}C(O)N(R^{N}), C(O)O, OC(O), OC(O)O, OC(O)N(R^{N}), —NR^{N}C(O)O, C(O)S, SC(O), C(═NR^{N}), C(═NR^{N})N(R^{N}), NR^{N}C(═NR^{N}), NR^{N}C(═NR^{N})N(R^{N}), —C(S), C(S)N(R^{N}), NR^{N}C(S), NR^{N}C(S)N(R^{N}), S(O), OS(O), S(O)O, OS(O)O, OS(O)₂, —S(O)₂O, OS(O)₂O, N(R^{N})S(O), S(O)N(R^{N}), N(R^{N})S(O)N(R^{N}), OS(O)N(R^{N}), N(R^{N})S(O)O, —S(O)₂, N(R^{N})S(O)₂, S(O)₂N(R^{N}), N(R^{N})S(O)₂N(R^{N}), OS(O)₂N(R^{N}), or N(R^{N})S(O)₂O; and

each instance of R^{N} is independently hydrogen, optionally substituted alkyl, or a nitrogen protecting group.

In certain embodiments, the compound of Formula (VIII) is of Formula (VIII-OH):

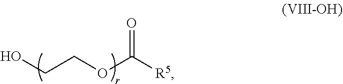

or a salt thereof. In some embodiments, r is 45. In other embodiments r is 1.

In certain embodiments, a compound of Formula (VIII) is of one of the following formulae:

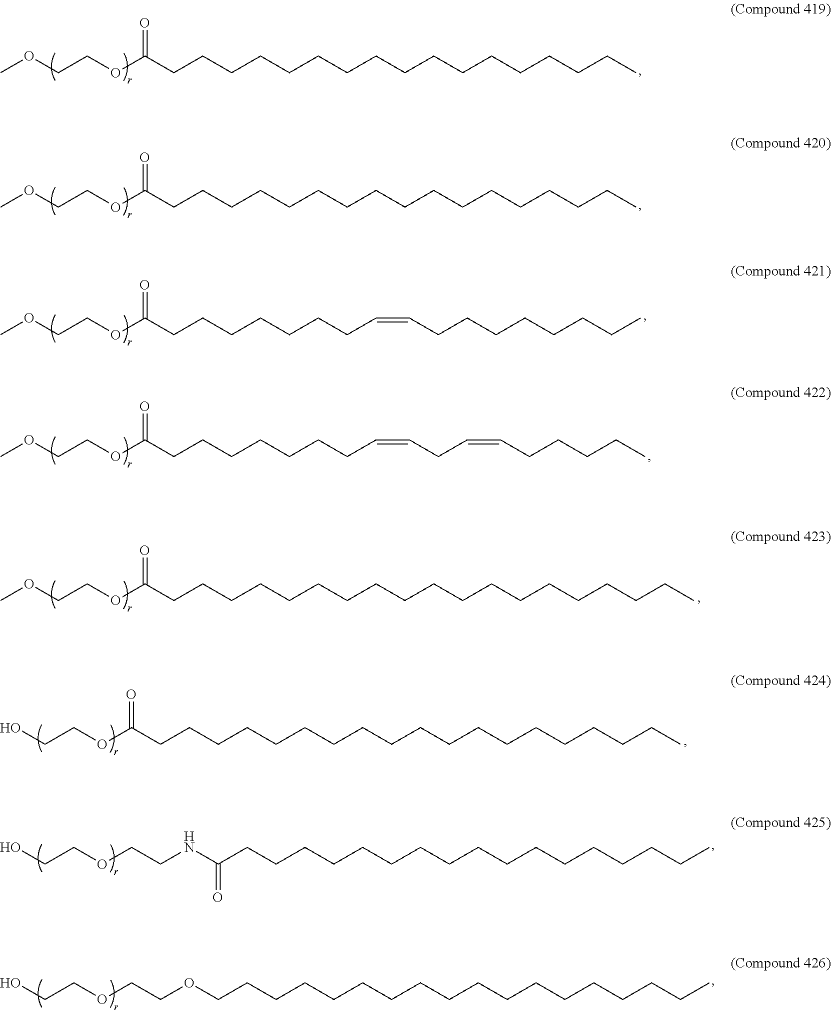

or a salt thereof. In some embodiments, r is 45.

In yet other embodiments the compound of Formula (VIII) is:

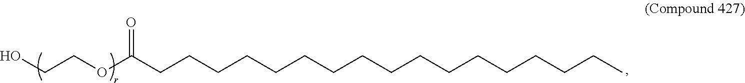

or a salt thereof.

In one embodiment, the compound of Formula (VIII) is

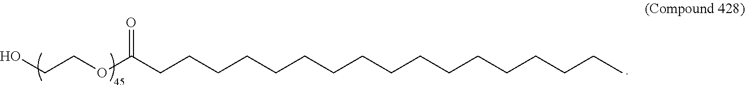

In one embodiment, the amount of PEG-lipid in the lipid composition of a pharmaceutical composition disclosed herein ranges from about 0.1 mol % to about 5 mol %, from about 0.5 mol % to about 5 mol %, from about 1 mol % to about 5 mol %, from about 1.5 mol % to about 5 mol %, from about 2 mol % to about 5 mol % mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %.

In one embodiment, the amount of PEG-lipid in the lipid composition disclosed herein is about 2 mol %. In one embodiment, the amount of PEG-lipid in the lipid composition disclosed herein is about 1.5 mol %.

In one embodiment, the amount of PEG-lipid in the lipid composition disclosed herein is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mol %.

In some aspects, the lipid composition of the pharmaceutical compositions disclosed herein does not comprise a PEG-lipid.

### Other Ionizable Amino Lipids

The lipid composition of the pharmaceutical composition disclosed herein can comprise one or more ionizable amino lipids in addition to or instead of a lipid according to Formula (I), (II), (III), (IV), (V), or (VI).

Ionizable lipids can be selected from the non-limiting group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethyl aminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), (13Z,165Z)-N,N-dimethyl-3-nonydocosa-13-16-dien-1-amine (L608), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, an ionizable amino lipid can also be a lipid including a cyclic amine group.

Ionizable lipids can also be the compounds disclosed in International Publication No. WO 2017/075531 A1, hereby incorporated by reference in its entirety. For example, the ionizable amino lipids include, but not limited to:

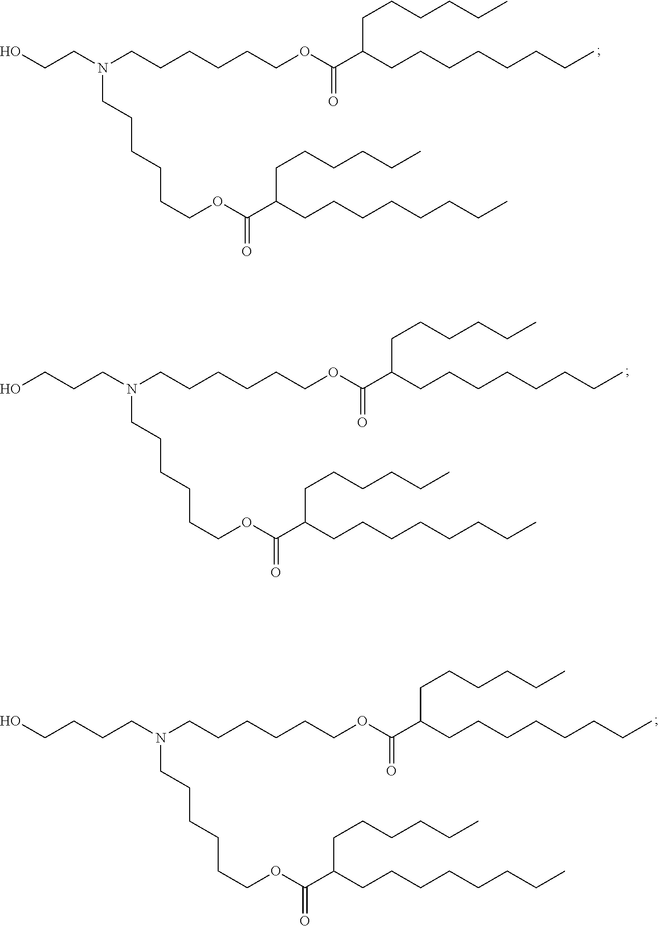 [SMILES: CCCCCCCCC(CCCCCC)C(=O)OCCCCCCN(CCO)CCCCCCOC(=O)C(CCCCCC)CCCCCCCC ; CCCCCCCCC(CCCCCC)C(=O)OCCCCCCN(CCCO)CCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC]

and any combination thereof.

Ionizable lipids can also be the compounds disclosed in International Publication No. WO 2015/199952 A1, hereby incorporated by reference in its entirety. For example, the ionizable amino lipids include, but not limited to:

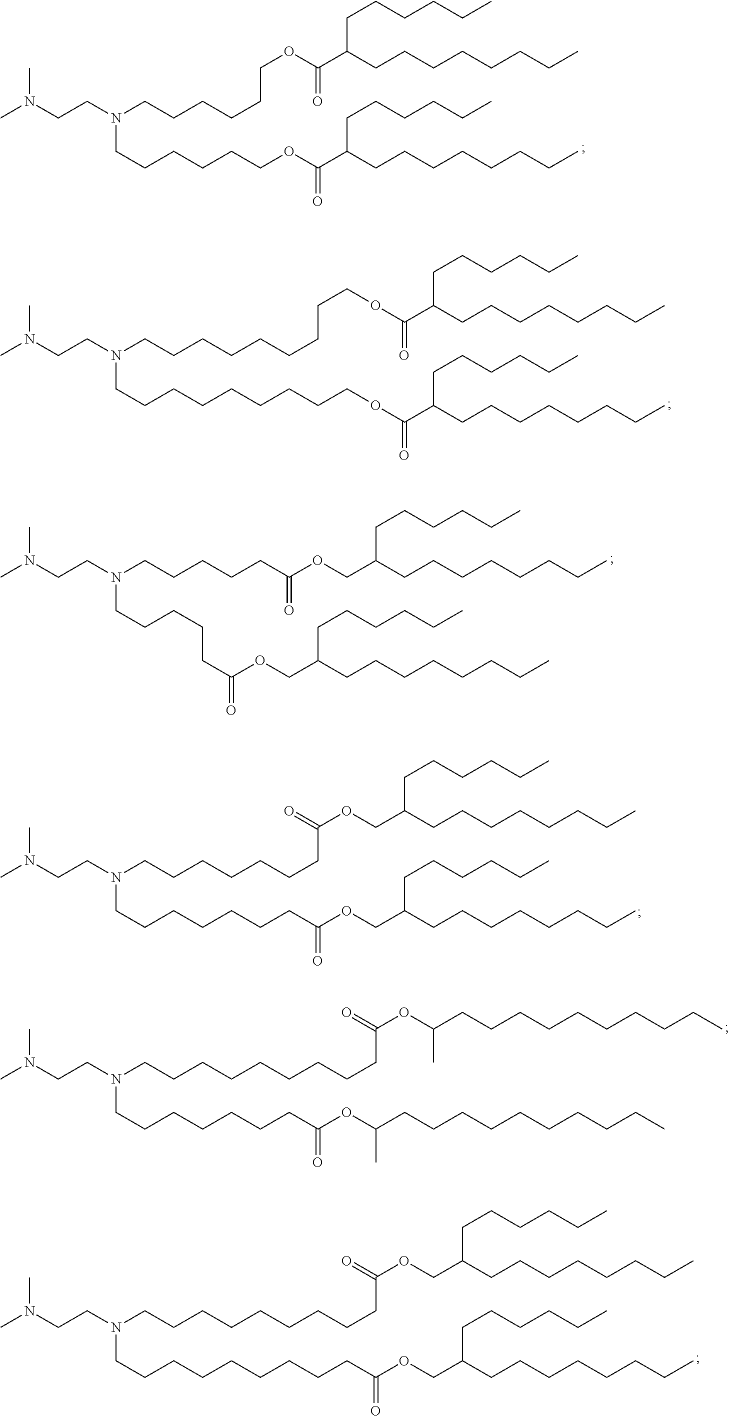

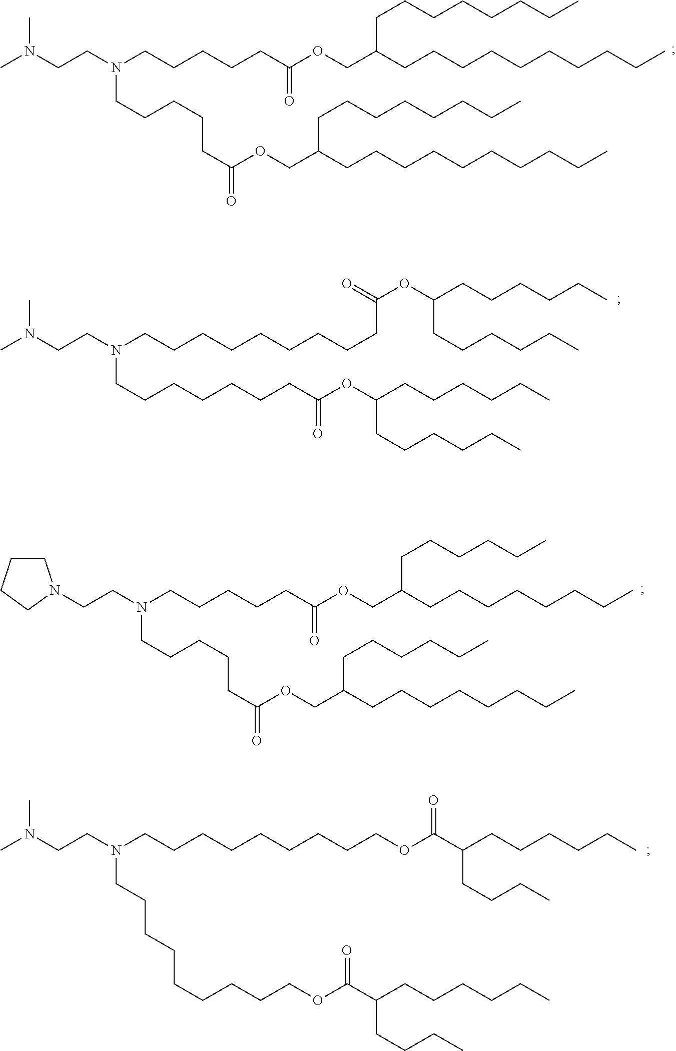

and any combination thereof.

### Nanoparticle Compositions

The lipid composition of a pharmaceutical composition disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof).

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

The ratio between the lipid composition and the polynucleotide range can be from about 10:1 to about 60:1 (wt/wt).

In some embodiments, the ratio between the lipid composition and the polynucleotide can be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1 or 60:1 (wt/wt). In some embodiments, the wt/wt ratio of the lipid composition to the polynucleotide encoding a therapeutic agent is about 20:1 or about 15:1.

In one embodiment, the lipid nanoparticles described herein can comprise polynucleotides (e.g., mRNA) in a lipid:polynucleotide weight ratio of 5:1, 10:1, 15:1, 20:1, 25:1, 30:1, 35:1, 40:1, 45:1, 50:1, 55:1, 60:1 or 70:1, or a range or any of these ratios such as, but not limited to, 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1,from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In one embodiment, the lipid nanoparticles described herein can comprise the polynucleotide in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

In some embodiments, the pharmaceutical compositions disclosed herein are formulated as lipid nanoparticles (LNP). Accordingly, the present disclosure also provides nanoparticle compositions comprising (i) a lipid composition comprising a delivery agent such as a compound of Formula (I) or (III) as described herein, and (ii) a polynucleotide encoding one or more cancer epitope polypeptides. In such nanoparticle composition, the lipid composition disclosed herein can encapsulate the polynucleotide encoding one or more cancer epitope polypeptides.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In some embodiments, the nanoparticle compositions of the present disclosure comprise at least one compound according to Formula (I), (III), (IV), (V), or (VI). For example, the nanoparticle composition can include one or more of Compounds 1-147, or one or more of Compounds 1-342. Nanoparticle compositions can also include a variety of other components. For example, the nanoparticle composition may include one or more other lipids in addition to a lipid according to Formula (I), (II), (III), (IV), (V), or (VI), such as (i) at least one phospholipid, (ii) at least one structural lipid, (iii) at least one PEG-lipid, or (iv) any combination thereof. Inclusion of structural lipid can be optional, for example when lipids according to formula III are used in the lipid nanoparticle compositions of the invention.

In some embodiments, the nanoparticle composition comprises a compound of Formula (I), (e.g., Compounds 18, 25, 26 or 48). In some embodiments, the nanoparticle composition comprises a compound of Formula (I) (e.g., Compounds 18, 25, 26 or 48) and a phospholipid (e.g., DSPC).

In some embodiments, the nanoparticle composition comprises a compound of Formula (III) (e.g., Compound 236). In some embodiments, the nanoparticle composition comprises a compound of Formula (III) (e.g., Compound 236) and a phospholipid (e.g., DOPE or DSPC).

In some embodiments, the nanoparticle composition comprises a lipid composition consisting or consisting essentially of compound of Formula (I) (e.g., Compounds 18, 25, 26 or 48). In some embodiments, the nanoparticle composition comprises a lipid composition consisting or consisting essentially of a compound of Formula (I) (e.g., Compounds 18, 25, 26 or 48) and a phospholipid (e.g., DSPC).

In some embodiments, the nanoparticle composition comprises a lipid composition consisting or consisting essentially of compound of Formula (III) (e.g., Compound 236). In some embodiments, the nanoparticle composition comprises a lipid composition consisting or consisting essentially of a compound of Formula (III) (e.g., Compound 236) and a phospholipid (e.g., DOPE or DSPC).

In one embodiment, a lipid nanoparticle comprises an ionizable lipid, a structural lipid, a phospholipid, and mRNA. In some embodiments, the LNP comprises an ionizable lipid, a PEG-modified lipid, a phospholipid and a structural lipid. In some embodiments, the LNP has a molar ratio of about 20-60% ionizable lipid:about 5-25% phospholipid:about 25-55% structural lipid; and about 0.5-15% PEG-modified lipid. In some embodiments, the LNP comprises a molar ratio of about 50% ionizable lipid, about 1.5% PEG-modified lipid, about 38.5% structural lipid and about 10% phospholipid. In some embodiments, the LNP comprises a molar ratio of about 55% ionizable lipid, about 2.5% PEG lipid, about 32.5% structural lipid and about 10% phospholipid. In some embodiments, the ionizable lipid is an ionizable amino lipid and the phospholipid is a neutral lipid, and the structural lipid is a cholesterol. In some embodiments, the LNP has a molar ratio of 50:38.5:10:1.5 of ionizable lipid: cholesterol: DSPC: PEG lipid. In some embodiments, the ionizable lipid is Compound 18 or Compound 236, and the PEG lipid is Compound 428.

In some embodiments, the LNP has a molar ratio of 50:38.5:10:1.5 of Compound 18:Cholesterol:Phospholipid:Compound 428. In some embodiments, the LNP has a molar ratio of 50:38.5:10:1.5 of Compound 18:Cholesterol:DSPC:Compound 428.

In some embodiments, the LNP has a molar ratio of 50:38.5:10:1.5 of Compound 236:Cholesterol:Phospholipid:Compound 428. In some embodiments, the LNP has a molar ratio of 50:38.5:10:1.5 of Compound 236:Cholesterol:DSPC:Compound 428.

In some embodiments, the LNP has a polydispersity value of less than 0.4. In some embodiments, the LNP has a net neutral charge at a neutral pH. In some embodiments, the LNP has a mean diameter of 50-150 nm. In some embodiments, the LNP has a mean diameter of 80-100 nm.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids leads them to form liposomes, vesicles, or membranes in aqueous media.

In some embodiments, a lipid nanoparticle (LNP) may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipids. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid, sometimes referred to in the art as an “ionizable cationic lipid”. In one embodiment, the ionizable amino lipid may have a positively charged hydrophilic head and a hydrophobic tail that are connected via a linker structure.

In addition to these, an ionizable lipid may also be a lipid including a cyclic amine group.

In one embodiment, the ionizable lipid may be selected from, but not limited to, a ionizable lipid described in International Publication Nos. WO2013086354 and WO2013116126; the contents of each of which are herein incorporated by reference in their entirety.

In yet another embodiment, the ionizable lipid may be selected from, but not limited to, formula CLI-CLXXXXII of U.S. Pat. No. 7,404,969; each of which is herein incorporated by reference in their entirety.

In one embodiment, the lipid may be a cleavable lipid such as those described in International Publication No. WO2012/170889, herein incorporated by reference in its entirety. In one embodiment, the lipid may be synthesized by methods known in the art and/or as described in International Publication Nos. WO2013/086354; the contents of each of which are herein incorporated by reference in their entirety.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

In some embodiments, the nanoparticle composition comprises a lipid composition consisting or consisting essentially of compound of Formula (I) (e.g., Compounds 18, 25, 26 or 48). In some embodiments, the nanoparticle composition comprises a lipid composition consisting or consisting essentially of a compound of Formula (I) (e.g., Compounds 18, 25, 26 or 48) and a phospholipid (e.g., DSPC or MSPC).

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide.

As used herein, “size” or “mean size” in the context of nanoparticle compositions refers to the mean diameter of a nanoparticle composition.

In one embodiment, the polynucleotide encoding one or more cancer epitope polypeptides are formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the nanoparticles have a diameter from about 10 to 500 nm. In one embodiment, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 m or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the zeta potential of the lipid nanoparticles can be from about 0 mV to about 100 mV, from about 0 mV to about 90 mV, from about 0 mV to about 80 mV, from about 0 mV to about 70 mV, from about 0 mV to about 60 mV, from about 0 mV to about 50 mV, from about 0 mV to about 40 mV, from about 0 mV to about 30 mV, from about 0 mV to about 20 mV, from about 0 mV to about 10 mV, from about 10 mV to about 100 mV, from about 10 mV to about 90 mV, from about 10 mV to about 80 mV, from about 10 mV to about 70 mV, from about 10 mV to about 60 mV, from about 10 mV to about 50 mV, from about 10 mV to about 40 mV, from about 10 mV to about 30 mV, from about 10 mV to about 20 mV, from about 20 mV to about 100 mV, from about 20 mV to about 90 mV, from about 20 mV to about 80 mV, from about 20 mV to about 70 mV, from about 20 mV to about 60 mV, from about 20 mV to about 50 mV, from about 20 mV to about 40 mV, from about 20 mV to about 30 mV, from about 30 mV to about 100 mV, from about 30 mV to about 90 mV, from about 30 mV to about 80 mV, from about 30 mV to about 70 mV, from about 30 mV to about 60 mV, from about 30 mV to about 50 mV, from about 30 mV to about 40 mV, from about 40 mV to about 100 mV, from about 40 mV to about 90 mV, from about 40 mV to about 80 mV, from about 40 mV to about 70 mV, from about 40 mV to about 60 mV, and from about 40 mV to about 50 mV. In some embodiments, the zeta potential of the lipid nanoparticles can be from about 10 mV to about 50 mV, from about 15 mV to about 45 mV, from about 20 mV to about 40 mV, and from about 25 mV to about 35 mV. In some embodiments, the zeta potential of the lipid nanoparticles can be about 10 mV, about 20 mV, about 30 mV, about 40 mV, about 50 mV, about 60 mV, about 70 mV, about 80 mV, about 90 mV, and about 100 mV.

The term “encapsulation efficiency” of a polynucleotide describes the amount of the polynucleotide that is encapsulated by or otherwise associated with a nanoparticle composition after preparation, relative to the initial amount provided. As used herein, “encapsulation” can refer to complete, substantial, or partial enclosure, confinement, surrounding, or encasement.

Encapsulation efficiency is desirably high (e.g., close to 100%). The encapsulation efficiency can be measured, for example, by comparing the amount of the polynucleotide in a solution containing the nanoparticle composition before and after breaking up the nanoparticle composition with one or more organic solvents or detergents.

Fluorescence can be used to measure the amount of free polynucleotide in a solution. For the nanoparticle compositions described herein, the encapsulation efficiency of a polynucleotide can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

The amount of a polynucleotide present in a pharmaceutical composition disclosed herein can depend on multiple factors such as the size of the polynucleotide, desired target and/or application, or other properties of the nanoparticle composition as well as on the properties of the polynucleotide.

For example, the amount of an mRNA useful in a nanoparticle composition can depend on the size (expressed as length, or molecular mass), sequence, and other characteristics of the mRNA. The relative amounts of a polynucleotide in a nanoparticle composition can also vary.

The relative amounts of the lipid composition and the polynucleotide present in a lipid nanoparticle composition of the present disclosure can be optimized according to considerations of efficacy and tolerability. For compositions including an mRNA as a polynucleotide, the N:P ratio can serve as a useful metric.

As the N:P ratio of a nanoparticle composition controls both expression and tolerability, nanoparticle compositions with low N:P ratios and strong expression are desirable. N:P ratios vary according to the ratio of lipids to RNA in a nanoparticle composition.

In general, a lower N:P ratio is preferred. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio can be from about 2:1 to about 8:1. In other embodiments, the N:P ratio is from about 5:1 to about 8:1. In certain embodiments, the N:P ratio is between 5:1 and 6:1. In one specific aspect, the N:P ratio is about is about 5.67:1.

In addition to providing nanoparticle compositions, the present disclosure also provides methods of producing lipid nanoparticles comprising encapsulating a polynucleotide. Such method comprises using any of the pharmaceutical compositions disclosed herein and producing lipid nanoparticles in accordance with methods of production of lipid nanoparticles known in the art. See, e.g., Wang et at (2015) “Delivery of oligonucleotides with lipid nanoparticles” Adv. Drug Deliv. Rev. 87:68-80; Silva et at (2015) “Delivery Systems for Biopharmaceuticals. Part I: Nanoparticles and Microparticles” Curr. Pharm. Technol. 16: 940-954; Naseri et at (2015) “Solid Lipid Nanoparticles and Nanostructured Lipid Carriers: Structure, Preparation and Application” Adv. Pharm. Bull. 5:305-13; Silva et al. (2015) “Lipid nanoparticles for the delivery of biopharmaceuticals” Curr. Pharm. Biotechnol. 16:291-302, and references cited therein.

### Kit Formulations

Kits for accomplishing these methods are also provided in other aspects of the invention. The kit includes a container housing a lipid nanoparticle formulation, a container housing a vaccine formulation, and instructions for adding a personalized mRNA cancer vaccine to the vaccine formulation to produce a personalized mRNA cancer vaccine formulation, mixing the personalized mRNA cancer vaccine formulation with the lipid nanoparticle formulation within 24 hours of administration to a subject. In some embodiments the kit includes a mRNA having an open reading frame encoding 2-100 cancer antigens.

The articles include pharmaceutical or diagnostic grade compounds of the invention in one or more containers. The article may include instructions or labels promoting or describing the use of the compounds of the invention.

As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the invention in connection with treatment of cancer.

“Instructions” can define a component of promotion, and typically involve written instructions on or associated with packaging of compositions of the invention. Instructions also can include any oral or electronic instructions provided in any manner.

Thus the agents described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended therapeutic application and the proper administration of these agents. In certain embodiments agents in a kit may be in a pharmaceutical formulation and dosage suitable for a particular application and for a method of administration of the agents.

The kit may be designed to facilitate use of the methods described herein by physicians and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for human administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container.

The kit may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The compositions of the kit may be provided as any suitable form, for example, as liquid solutions or as dried powders. When the composition provided is a dry powder, the powder may be reconstituted by the addition of a suitable solvent, which may also be provided. In embodiments where liquid forms of the composition are sued, the liquid form may be concentrated or ready to use. The solvent will depend on the compound and the mode of use or administration. Suitable solvents for drug compositions are well known and are available in the literature. The solvent will depend on the compound and the mode of use or administration.

The kits, in one set of embodiments, may comprise a carrier means being compartmentalized to receive in close confinement one or more container means such as vials, tubes, and the like, each of the container means comprising one of the separate elements to be used in the method. For example, one of the containers may comprise a positive control for an assay. Additionally, the kit may include containers for other components, for example, buffers useful in the assay.

The present invention also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, topical or mucosal delivery.

In a preferred embodiment, the unit dosage form is suitable for intravenous, intramuscular or subcutaneous delivery. Thus, the invention encompasses solutions, preferably sterile, suitable for each delivery route.

In another preferred embodiment, compositions of the invention are stored in containers with biocompatible detergents, including but not limited to, lecithin, taurocholic acid, and cholesterol; or with other proteins, including but not limited to, gamma globulins and serum albumins. More preferably, compositions of the invention are stored with human serum albumins for human uses, and stored with bovine serum albumins for veterinary uses.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures (such as methods for monitoring mean absolute lymphocyte counts, tumor cell counts, and tumor size) and other monitoring information.

More specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material. The invention also provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material. The invention further provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material. The invention further provides an article of manufacture comprising a needle or syringe, preferably packaged in sterile form, for injection of the formulation, and/or a packaged alcohol pad.

Relative amounts of the active ingredient, the pharmaceutically acceptable excipient, and/or any additional ingredients in a vaccine composition may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the package containing the pharmaceutical product contains 0.1 mg to 1 mg of mRNA. In some embodiments, the package containing the pharmaceutical product contains 0.35 mg of mRNA. In some embodiments, the concentration of the mRNA is 1 mg/mL.

In some embodiments, the package containing the pharmaceutical product contains contains 5-15 mg of total lipid. In some embodiments, the package containing the pharmaceutical product contains contains 7 mg of total lipid. In some embodiment, the concentration of total lipid is 20 mg/mL.

In some embodiments, the RNA (e.g., mRNA) vaccine compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see e.g., the range of unit doses described in International Publication No. WO2013/078199, herein incorporated by reference in its entirety). In some embodiments, the RNA (e.g., mRNA) vaccine is administered at a dosage level sufficient to deliver 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. In some embodiments, the RNA (e.g., mRNA) vaccine is administered at a dosage level sufficient to deliver between 10 μg and 400 μg of the mRNA vaccine to the subject. In some embodiments, the RNA (e.g., mRNA) vaccine is administered at a dosage level sufficient to deliver 0.033 mg, 0.1 mg, 0.2 mg, or 0.4 mg to the subject.

The desired dosage may be delivered three times a day, two times a day, once a day, every other day, every third day, every week, every two weeks, every three weeks, every four weeks, every 2 months, every three months, every 6 months, etc. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In some embodiments, the RNA vaccine compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg. In some embodiments, the RNA vaccine compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, the RNA vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, a the RNA vaccine composition may be administered three or four times, or more. In some embodiments, the mRNA vaccine composition is administered once a day every three weeks

In some embodiments, the RNA vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg, 0.025 mg, 0.100 mg or 0.400 mg.

In some embodiments the RNA vaccine for use in a method of vaccinating a subject is administered the subject a single dosage of between 10 μg/kg and 400 μg/kg of the nucleic acid vaccine in an effective amount to vaccinate the subject. In some embodiments the RNA vaccine for use in a method of vaccinating a subject is administered the subject a single dosage of between 10 μg and 400 μg of the nucleic acid vaccine in an effective amount to vaccinate the subject.

In some embodiments, the RNA vaccine composition may comprise the polynucleotide described herein, formulated in a lipid nanoparticle comprising MC3, Cholesterol, DSPC and PEG2000-DMG, the buffer trisodium citrate, sucrose and water for injection. As a non-limiting example, the composition comprises: 2.0 mg/mL of drug substance (e.g., polynucleotides encoding cancer antigens), 21.8 mg/mL of MC3, 10.1 mg/mL of cholesterol, 5.4 mg/mL of DSPC, 2.7 mg/mL of PEG2000-DMG, 5.16 mg/mL of trisodium citrate, 71 mg/mL of sucrose and 1.0 mL of water for injection.

In some embodiments, a nanoparticle (e.g., a lipid nanoparticle) has a mean diameter of 10-500 nm, 20-400 nm, 30-300 nm, 40-200 nm. In some embodiments, a nanoparticle (e.g., a lipid nanoparticle) has a mean diameter of 50-150 nm, 50-200 nm, 80-100 nm or 80-200 nm.

In some embodiments, the RNA vaccine comprises 5-15 mg of total lipid, e.g., 5, 6, 7, 8, 9, 10, 11, 12, 13, 14 or 15 mg of total lipid. In some embodiments, the RNA vaccine comprises 7 mg of total lipid. In some embodiment, the concentration of total lipid in the vaccine formulation is 10-30 mg/mL, e.g., 10, 11, 12, 13, 14, 15, 16, 17, 18, 19, 20, 21, 22, 23, 24, 25, 26, 27, 28, 29, or 30 mg/mL.

Flagellin is an approximately 500 amino acid monomeric protein that polymerizes to form the flagella associated with bacterial motion. Flagellin is expressed by a variety of flagellated bacteria (Salmonella typhimurium for example) as well as non-flagellated bacteria (such as Escherichia coli). Sensing of flagellin by cells of the innate immune system (dendritic cells, macrophages, etc.) is mediated by the Toll-like receptor 5 (TLR5) as well as by Nod-like receptors (NLRs) Ipaf and Naip5. TLRs and NLRs have been identified as playing a role in the activation of innate immune response and adaptive immune response. As such, flagellin provides an adjuvant effect in a vaccine.

The nucleotide and amino acid sequences encoding known flagellin polypeptides are publicly available in the NCBI GenBank database. The flagellin sequences from S. Typhimurium, H. Pylori, V. Cholera, S. marcesens, S. flexneri, T. pallidum, L. pneumophila, B. burgdorferei, C. difficile, R. meliloti, A. tumefaciens, R. lupini, B. clarridgeiae, P. Mirabilis, B. subtilus, L. monocytogenes, P. aeruginosa, and E. coli, among others are known.

A flagellin polypeptide, as used herein, refers to a full length flagellin protein, immunogenic fragments thereof, and peptides having at least 50% sequence identity to a flagellin protein or immunogenic fragments thereof. Exemplary flagellin proteins include flagellin from Salmonella typhi (UniPro Entry number: Q56086), Salmonella typhimurium (A0A0C9DG09), Salmonella enteritidis (A0A0C9BAB7), and Salmonella choleraesuis (Q6V2X8. In some embodiments, the flagellin polypeptide has at least 60%, 70%, 75%, 80%, 90%, 95%, 97%, 98%, or 99% sequence identity to a flagellin protein or immunogenic fragments thereof.

In some embodiments, the flagellin polypeptide is an immunogenic fragment. An immunogenic fragment is a portion of a flagellin protein that provokes an immune response. In some embodiments, the immune response is a TLR5 immune response. An example of an immunogenic fragment is a flagellin protein in which all or a portion of a hinge region has been deleted or replaced with other amino acids. For example, an antigenic polypeptide may be inserted in the hinge region. Hinge regions are the hypervariable regions of a flagellin. Hinge regions of a flagellin are also referred to as “D3 domain or region,” “propeller domain or region,” “hypervariable domain or region” and “variable domain or region.” “At least a portion of a hinge region,” as used herein, refers to any part of the hinge region of the flagellin, or the entirety of the hinge region. In other embodiments an immunogenic fragment of flagellin is a 20, 25, 30, 35, or 40 amino acid C-terminal fragment of flagellin.

The flagellin monomer is formed by domains D0 through D3. D0 and D1, which form the stem, are composed of tandem long alpha helices and are highly conserved among different bacteria. The D1 domain includes several stretches of amino acids that are useful for TLR5 activation. The entire D1 domain or one or more of the active regions within the domain are immunogenic fragments of flagellin. Examples of immunogenic regions within the D1 domain include residues 88-114 and residues 411-431 in Salmonella typhimurium FliC flagellin. Within the 13 amino acids in the 88-100 region, at least 6 substitutions are permitted between Salmonella flagellin and other flagellins that still preserve TLR5 activation. Thus, immunogenic fragments of flagellin include flagellin like sequences that activate TLR5 and contain a 13 amino acid motif that is 53% or more identical to the Salmonella sequence in 88-100 of FliC (LQRVRELAVQSAN; SEQ ID NO: 356).

In some embodiments, the RNA (e.g., mRNA) vaccine includes an RNA that encodes a fusion protein of flagellin and one or more antigenic polypeptides. A “fusion protein” as used herein, refers to a linking of two components of the construct. In some embodiments, a carboxy-terminus of the antigenic polypeptide is fused or linked to an amino terminus of the flagellin polypeptide. In other embodiments, an amino-terminus of the antigenic polypeptide is fused or linked to a carboxy-terminus of the flagellin polypeptide. The fusion protein may include, for example, one, two, three, four, five, six or more flagellin polypeptides linked to one, two, three, four, five, six or more antigenic polypeptides. When two or more flagellin polypeptides and/or two or more antigenic polypeptides are linked such a construct may be referred to as a “multimer.”

Each of the components of a fusion protein may be directly linked to one another or they may be connected through a linker. For instance, the linker may be an amino acid linker. The amino acid linker encoded for by the RNA (e.g., mRNA) vaccine to link the components of the fusion protein may include, for instance, at least one member selected from the group consisting of a lysine residue, a glutamic acid residue, a serine residue and an arginine residue. In some embodiments the linker is 1-30, 1-25, 1-25, 5-10, 5, 15, or 5-20 amino acids in length.

### Modes of Vaccine Administration

Cancer RNA vaccines may be administered by any route which results in a therapeutically effective outcome. These include, but are not limited, to intradermal, intramuscular, and/or subcutaneous administration. The present disclosure provides methods comprising administering RNA vaccines to a subject in need thereof. The exact amount required will vary from subject to subject, depending on the species, age, and general condition of the subject, the severity of the disease, the particular composition, its mode of administration, its mode of activity, and the like. Cancer RNA vaccines compositions are typically formulated in dosage unit form for ease of administration and uniformity of dosage. It will be understood, however, that the total daily usage of cancer RNA vaccines compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, cancer RNA vaccines compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see e.g., the range of unit doses described in International Publication No WO2013/078199, herein incorporated by reference in its entirety). The desired dosage may be delivered three times a day, two times a day, once a day, every other day, every third day, every week, every two weeks, every three weeks, every four weeks, every 2 months, every three months, every 6 months, etc. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In exemplary embodiments, cancer RNA vaccines compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg.

A RNA vaccine pharmaceutical composition described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

### Example 1. Manufacture of Polynucleotides

According to the present disclosure, the manufacture of polynucleotides and or parts or regions thereof may be accomplished utilizing the methods taught in International Application WO2014/152027 entitled “Manufacturing Methods for Production of RNA Transcripts”, the contents of which is incorporated herein by reference in its entirety.

Purification methods may include those taught in International Application WO2014/152030 and WO2014/152031, each of which is incorporated herein by reference in its entirety.

Detection and characterization methods of the polynucleotides may be performed as taught in WO2014/144039, which is incorporated herein by reference in its entirety.

Characterization of the polynucleotides of the disclosure may be accomplished using a procedure selected from the group consisting of polynucleotide mapping, reverse transcriptase sequencing, charge distribution analysis, and detection of RNA impurities, wherein characterizing comprises determining the RNA transcript sequence, determining the purity of the RNA transcript, or determining the charge heterogeneity of the RNA transcript. Such methods are taught in, for example, WO2014/144711 and WO2014/144767, the contents of each of which is incorporated herein by reference in its entirety.

### Example 2 Chimeric Polynucleotide Synthesis

### Introduction

According to the present disclosure, two regions or parts of a chimeric polynucleotide may be joined or ligated using triphosphate chemistry.

According to this method, a first region or part of 100 nucleotides or less is chemically synthesized with a 5′ monophosphate and terminal 3′desOH or blocked OH. If the region is longer than 80 nucleotides, it may be synthesized as two strands for ligation.

If the first region or part is synthesized as a non-positionally modified region or part using in vitro transcription (IVT), conversion the 5′monophosphate with subsequent capping of the 3′ terminus may follow.

Monophosphate protecting groups may be selected from any of those known in the art.

The second region or part of the chimeric polynucleotide may be synthesized using either chemical synthesis or IVT methods. IVT methods may include an RNA polymerase that can utilize a primer with a modified cap. Alternatively, a cap of up to 130 nucleotides may be chemically synthesized and coupled to the IVT region or part.

The entire chimeric polynucleotide need not be manufactured with a phosphate-sugar backbone. If one of the regions or parts encodes a polypeptide, then it is preferable that such region or part comprise a phosphate-sugar backbone.

Ligation is then performed using any known click chemistry, orthoclick chemistry, solulink, or other bioconjugate chemistries known to those in the art.

### Synthetic Route

The chimeric polynucleotide is made using a series of starting segments. Such segments include:

(a) Capped and protected 5′ segment comprising a normal 3′OH (SEG. 1)

(b) 5′ triphosphate segment which may include the coding region of a polypeptide and comprising a normal 3′OH (SEG. 2)

(c) 5′ monophosphate segment for the 3′ end of the chimeric polynucleotide (e.g., the tail) comprising cordycepin or no 3′OH (SEG. 3)

After synthesis (chemical or IVT), segment 3 (SEG. 3) is treated with cordycepin and then with pyrophosphatase to create the 5′monophosphate.

Segment 2 (SEG. 2) is then ligated to SEG. 3 using RNA ligase. The ligated polynucleotide is then purified and treated with pyrophosphatase to cleave the diphosphate. The treated SEG. 2-SEG. 3 construct is then purified and SEG. 1 is ligated to the 5′ terminus. A further purification step of the chimeric polynucleotide may be performed.

The yields of each step may be as much as 90-95%.

### Example 3: PCR for cDNA Production

PCR procedures for the preparation of cDNA are performed using 2×KAPA HIFI™ HotStart ReadyMix by Kapa Biosystems (Woburn, Mass.). This system includes 2×KAPA ReadyMixl2.5 μl; Forward Primer (10 μM) 0.75 μl; Reverse Primer (10 μM) 0.75 μl; Template cDNA −100 ng; and dH₂0 diluted to 25.0 μl. The reaction conditions are at 95° C. for 5 min. and 25 cycles of 98° C. for 20 sec, then 58° C. for 15 sec, then 72° C. for 45 sec, then 72° C. for 5 min. then 4° C. to termination.

The reaction is cleaned up using Invitrogen's PURELINK™ PCR Micro Kit (Carlsbad, Calif.) per manufacturer's instructions (up to 5 μg). Larger reactions will require a cleanup using a product with a larger capacity. Following the cleanup, the cDNA is quantified using the NANODROP™ and analyzed by agarose gel electrophoresis to confirm the cDNA is the expected size. The cDNA is then submitted for sequencing analysis before proceeding to the in vitro transcription reaction.

### Example 4. In Vitro Transcription (IVT)

The in vitro transcription reaction generates polynucleotides containing uniformly modified polynucleotides. Such uniformly modified polynucleotides may comprise a region or part of the polynucleotides of the disclosure. The input nucleotide triphosphate (NTP) mix is made in-house using natural and un-natural NTPs.

A typical in vitro transcription reaction includes the following:

| 1 | Template cDNA | 1.0 | μg |
|---|---|---|---|
| 2 | 10x transcription buffer (400 mM Tris-HCl | 2.0 | μl |
| | pH 8.0, 190 mM MgCl₂, | | |
| | 50 mM DTT, 10 mM Spermidine) | | |
| 3 | Custom NTPs (25 mM each) | 7.2 | μl |
| 4 | RNase Inhibitor | 20 | U |
| 5 | T7 RNA polymerase | 3000 | U |
| 6 | dH₂0 | Up to 20.0 μl. and | |
| 7 | Incubation at 37° C. for 3 hr-5 hrs. | | |

The crude IVT mix may be stored at 4° C. overnight for cleanup the next day. 1 U of RNase-free DNase is then used to digest the original template. After 15 minutes of incubation at 37° C., the mRNA is purified using Ambion's MEGACLEAR™ Kit (Austin, Tex.) following the manufacturer's instructions. This kit can purify up to 500 μg of RNA. Following the cleanup, the RNA is quantified using the NanoDrop and analyzed by agarose gel electrophoresis to confirm the RNA is the proper size and that no degradation of the RNA has occurred.

### Example 5. STING Studies

In this example, whether an immune potentiator, such as constitutively active STING, can boost T-cell responses to a concatameric vaccine was investigated. An mRNA construct encoding the RNA 31 concatemer, which encodes Class I and Class II epitopes, was used as the vaccine and the effect of STING on T-cell responses to Class I and Class II epitopes was investigated. The RNA 31 and STING mRNAs were either coformulated and delivered simultaneously, or were not coformulated, with delayed delivery of STING mRNA. Animals were given a priming dose on Day 1 and a boost on Day 15. Splenocytes were harvested on Day 22.

Different materials were tested in order to determine the immunogenicity when adding STING at various ratios to a concatemeric vaccine, to compare STING to top-ranked commercially available adjuvants, to determine whether the immunogenicity is dependent upon the timing of STING dosing, and to examine the immunogenicity of unformulated mRNA when dosed with STING. The following materials/conditions were tested: RNA 31 (3 μg), RNA 31 (3 μg) with Poly I:C (10 μg), RNA 31 (3 μg) with MPLA (5 μg), STING (1 μg)/RNA 31 (3 μg), STING (0.6 μg)/RNA 31 (3 μg), STING (0.6 μg)/RNA 54 (3 μg), STING (0.6 μg)/RNA 31 (3 μg) (24 hours later), STING (0.6 μg)/RNA 31 (3 μg) (48 hours later), STING (0.6 μg)/RNA 31 (3 μg) (unformulated), and STING (6 μg)/RNA 31 (30 μg) (unformulated). CA-54 is a concatemer of 5 Class II epitopes (all of which are contained within RNA 31).

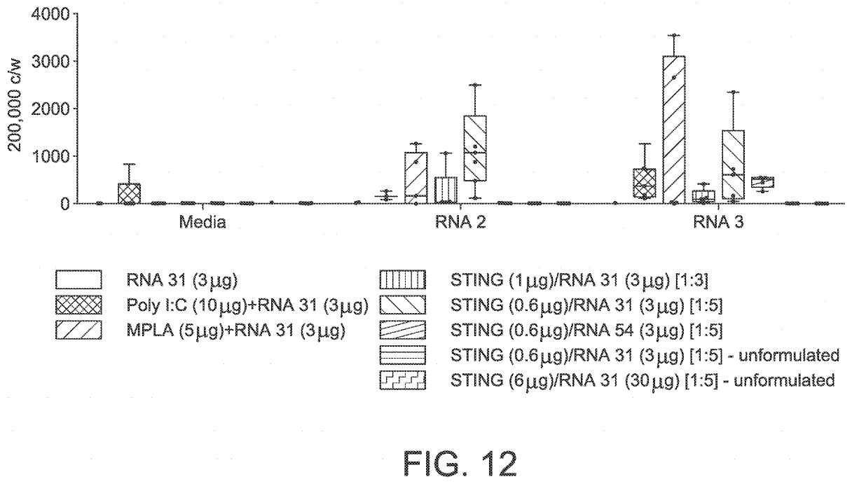

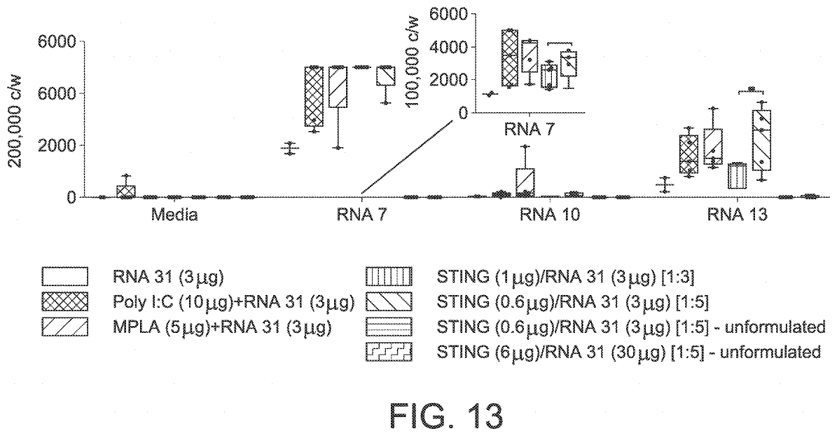

Results are shown in FIGS. 12-13. When the antigen-specific IFNγ responses were examined with Class II epitopes STING was found to boost the immune response to the MHC class II epitopes encoded by mRNA. STING behaved comparably to commercially available adjuvants (5-10 fold difference in dose). Although both ratios tested worked, the 1:5 STING:antigen ratio performed better than 1:3 combination (FIG. 12). Similar results were obtained using Class I epitopes as described above and shown in FIG. 13. Likewise, the 1:5 STING:antigen ratio was found to perform better than the 1:3 combination for class I epitopes.

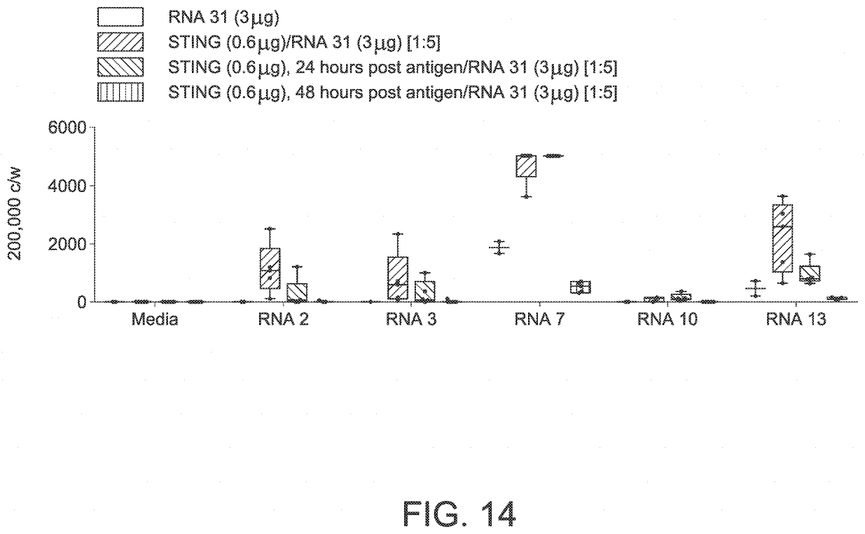

Further, it was found that dosing STING at a later time point (24 hours) had similar iimmunogenicity to codelivery (FIG. 14).

In a further experiment, the effect of different STING-to-antigen ratios was examined using 52 murine epitopes (adding eptioes_4a_DXRX_perm). Mice received a prime dose on Day 1, a boost dose on Day 8, and splenocytes were harvested on Day 15. T cell responses to re-stimulation were evaluated using ELISpot and FACS. Restimulation was performed with peptide sequences corresponding to epitopes eocnding the concatamer. T cell response to two Class II epitopes (RNA 2, RNA 3) and four Class I epitopes (RNA 7, RNA 10, RNA 13, RNA 22) were examined.

Quite surprisingly, it was found that the addition of STING across the majority of ratios tested improved T cell responses compared to antigen alone and never performed worse than antigen alone. The breadth of responsiveness was unexpected. For four of the six antigens (epitopes) tested, the addition of STING to antigen at the 10-30ug total dose consistently produced higher T cell responses than that of the 50ug dose of antigen alone. Thus, there is a wide bell curve in the ratio of STING:antigen for improved immunogenicity.

The study groups were as shown in the following table:

| STING:AG | | | | | | | | | | | | |
|---|---|---|---|---|---|---|---|---|---|---|---|---|
| Ratio | 0:1 | | 20:1 | | 5:1 | | 1:1 | | 1:5 | | 1:20 | |
| Total mRNA | [symbol] | AG | STING | AG | STING | AG | STING | AG | STING | AG | STING | AG |
| μg | (μg) | (μg) | (μg) | (μg) | (μg) | (μg) | (μg) | (μg) | (μg) | (μg) | (μg) | (μg) |
| 0.15 | 2.85 | 0.15 | | | | | | | | | | |
| 0.5 | 9.5 | 0.5 | | | | | | | | | | |
| 1.5 | 28.6 | 1.5 | | | | | | | | | | |
| 3 | 27 | 3 | 2.85 | 0.15 | 2.4 | 0.6 | 1.5 | 1.5 | 0.6 | 2.4 | 0.15 | 2.85 |
| 10 | 20 | 10 | 9.5 | 0.5 | 8.3 | 1.4 | 5.0 | 5.0 | 1.4 | 8.3 | 0.5 | 9.5 |
| 30 | 0 | 30 | 28.6 | 1.4 | 25.0 | 4.2 | 15.0 | 15.0 | 4.2 | 25.0 | 1.4 | 28.6 |
| 50 | 0 | 50 | | | | | | | | | | |

[symbol]

indicates data missing or illegible when filed

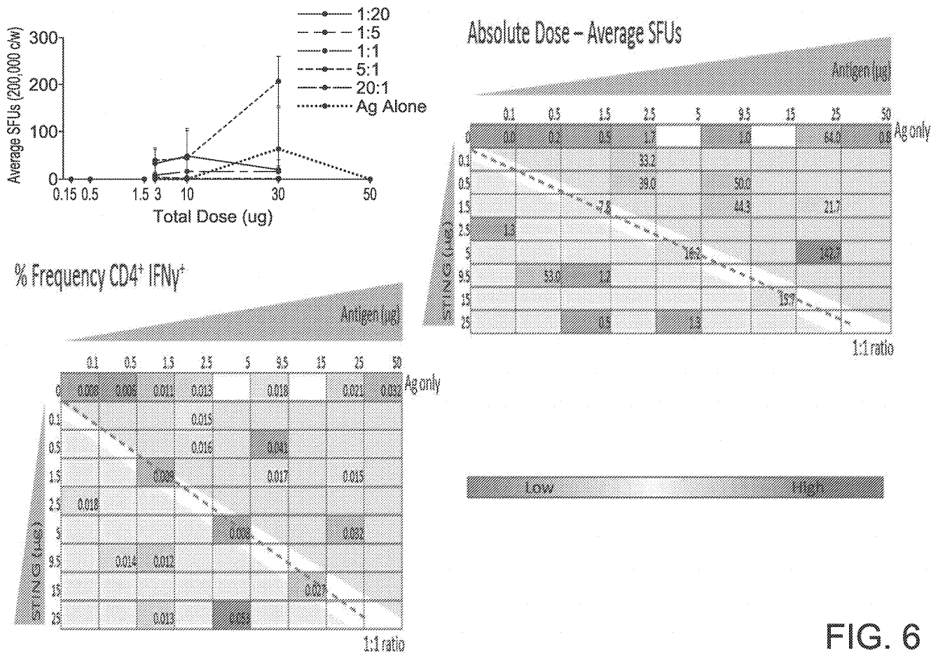

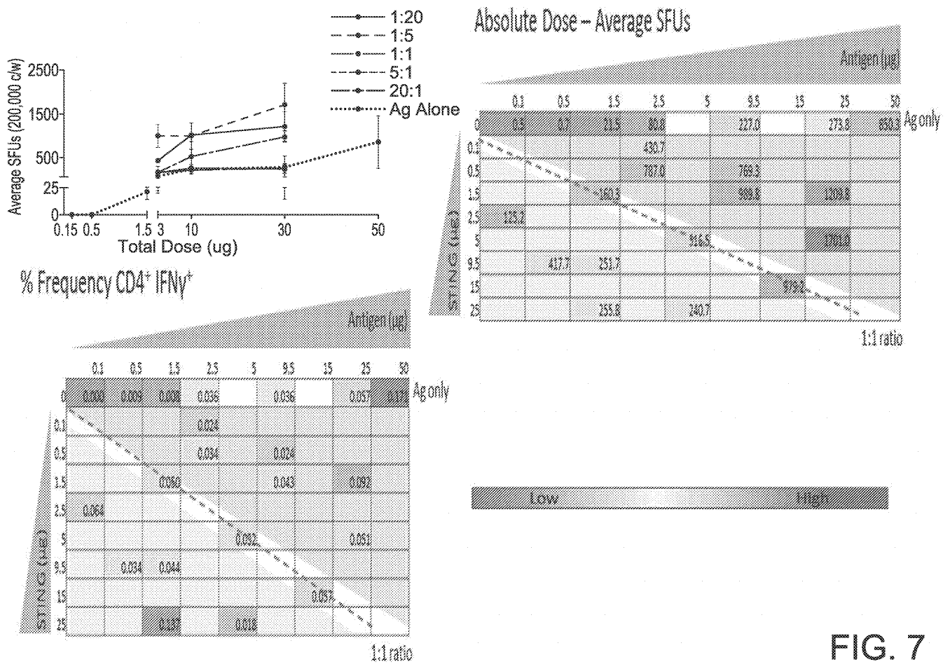

Among the Class II epitopes, RNA 2 (results shown in FIG. 6) and RNA 3 (results shown in FIG. 7) showed that adding STING increased T cell responses at ratios less than 1:1 (STING:antigen) relative to the antigen only group, including at doses up to 50 μg antigen alone. The left panel of FIG. 7 shows that adding STING increased T cell response at all ratios relative to the antigen only group.

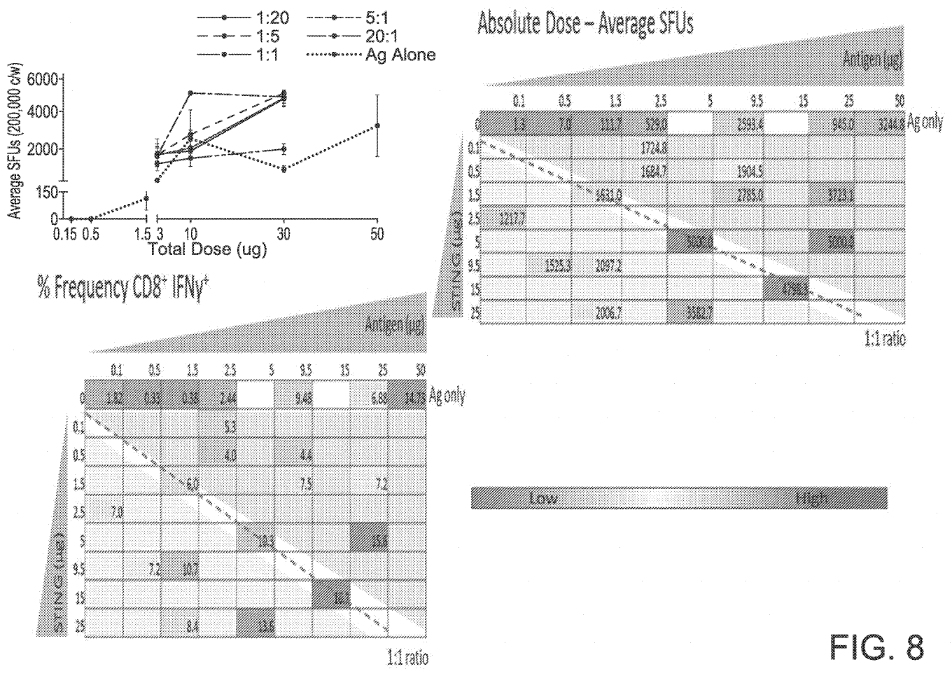

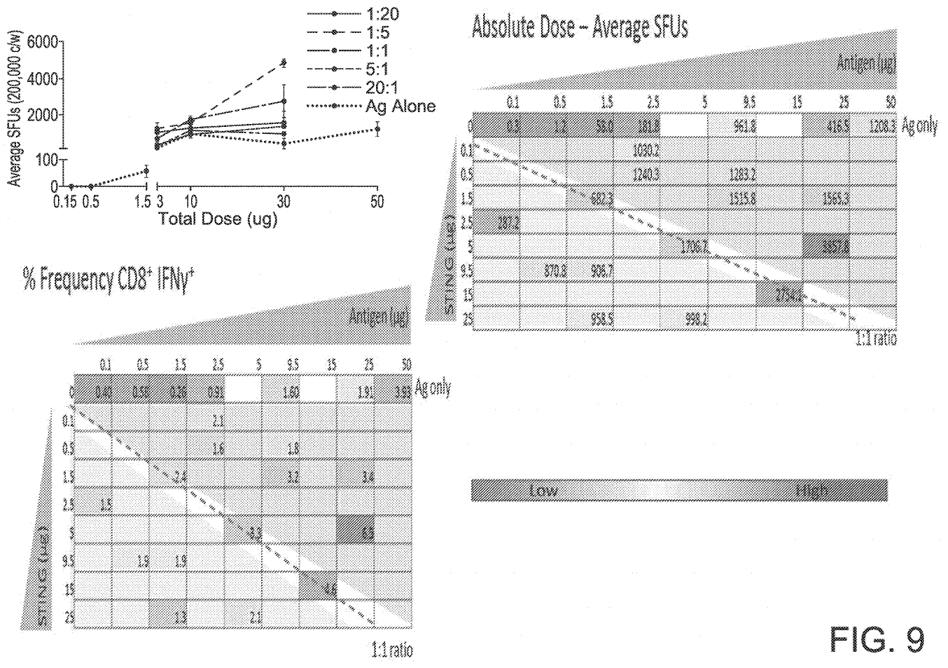

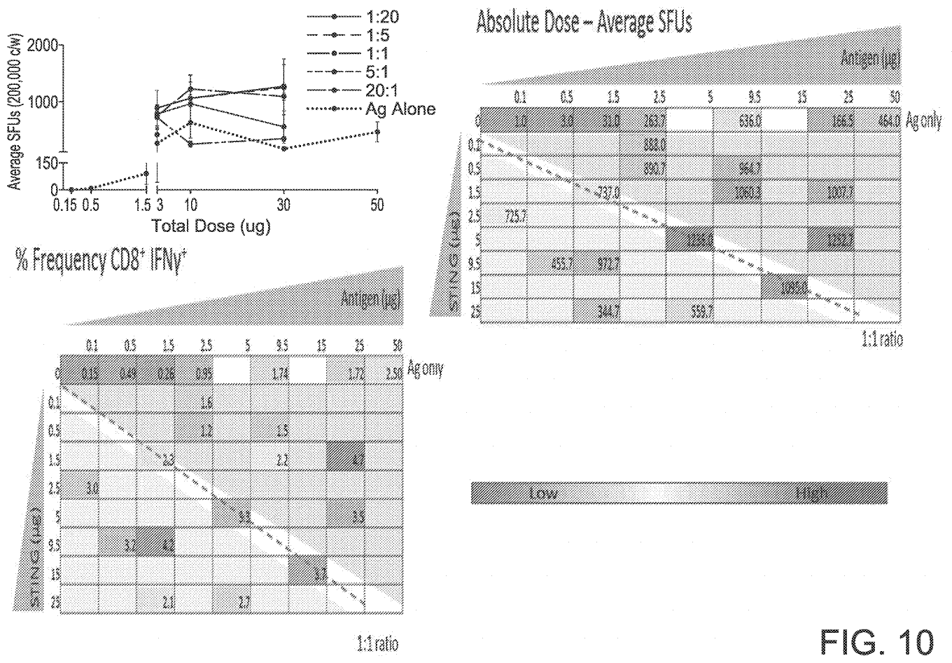

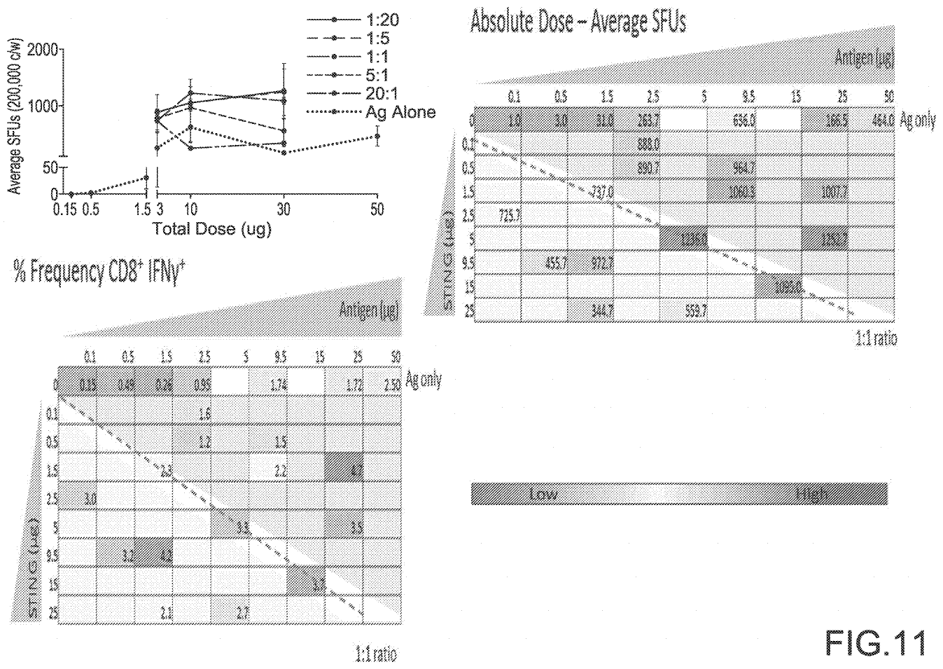

Similar results were seen with the Class I epitopes. RNA 7 (results shown in FIG. 8), RNA 13 (results shown in FIG. 9), RNA 22 (results shown in FIG. 10), and RNA 10 (results shown in FIG. 11) all showed that ratios of STING:antigen produced higher T cell responses relative to the antigen only group when compared to the total mRNA dose.

### Example 6. Concatamer Studies

Studies were conducted to examine whether full read through of longer constructs was possible and to compare immunogenicity to epitopes contained in 20 and 52 epitope constructs. For the experiments, five groups of different formulations were tested in LNPs containing Compound 257:

| | | | Class II | | Class I |
|---|---|---|---|---|---|
| | | | (number of | | (number of |
| | | Final | constructs - | | constructs - |
| | Test/Control | Concen- | number of | | number of |
| Group | Material | tration | amino acids) | | amino acids) |
| 1 | RNA 31 | 3 | 5-31 | aa | 15-31aa |
| 2 | 20 epitopes_21 flanks | 3 | 5-21 | aa | 15-21aa |
| 3 | 20 epitopes_21 flank | 3 | 5-21 | aa | 15-15aa |
| | Class II_15 flank | | | | |
| | Class I | | | | |
| 4 | 52 epitopes_21 flanks | 7.5 | 13-21 | aa | 39-21aa |
| 5 | 52 eptiopes_21 flank | 7.5 | 13-21 | aa | 39-15aa |
| | Class II_15 flank | | | | |
| | Class I | | | | |

Dosing was equi-picomolar, meaning that all groups received the same concentration of each individual epitope despite construct length. Animals were given one dose on day 0 (priming dose), a second dose on day 6 (boost), and then splenocytes were harvested on day 12 and IFNγ ELISpot was performed on samples.

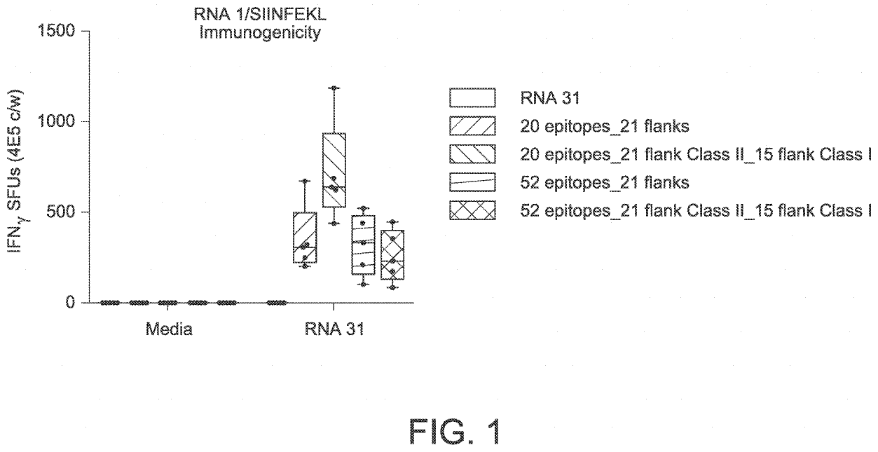

The immunogenicity of the 52 epitope-containing vaccine was examined. RNA 1/SIINFEKL (SEQ ID NO: 231) was the final epitope for each of the four constructs tested. SIINFEKL (SEQ ID NO: 231) T-cell responses in 52 epitope constructs confirm the full read through of the concatamer, as INFγ responses were observed from all test groups when re-stimulation with RNA 1/SIINFEKL (SEQ ID NO: 231) was performed (FIG. 1). Note that, as expected, there was no RNA 1 found in the RNA 31 concatamer because the concatamer did not have the RNA 1/SIINFEKL (SEQ ID NO: 231) epitope.

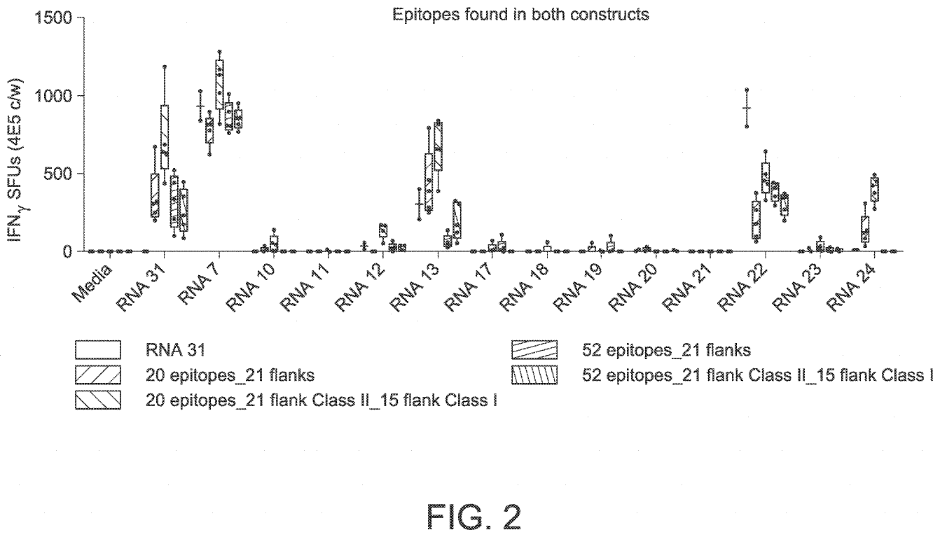

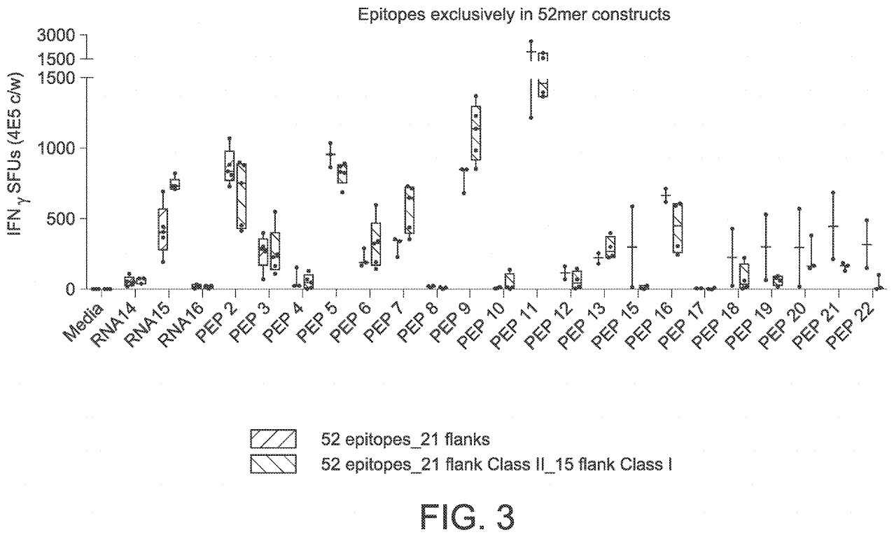

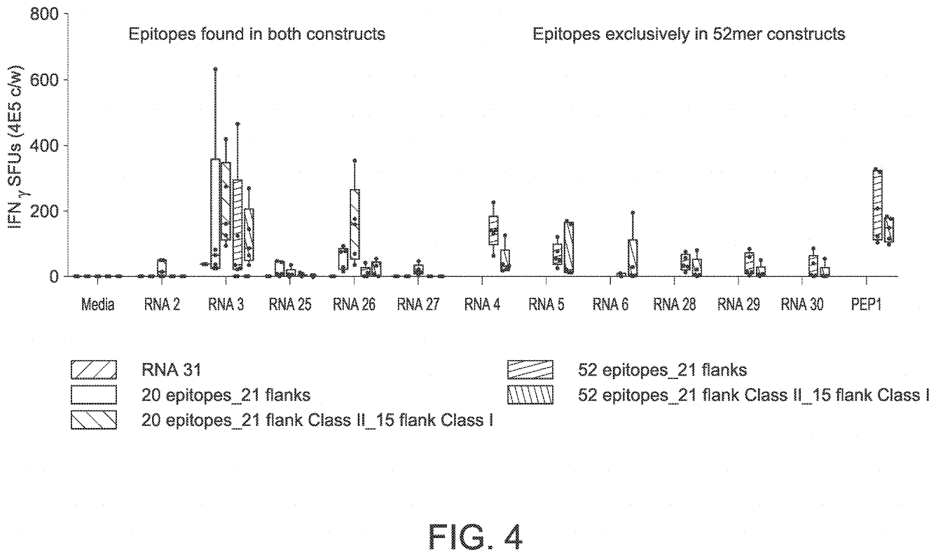

The immunogenicity between the 52 mer and 20 mer constructs was similar. For example, both behave similarly when re-stimulated with Class I epitopes (FIG. 2) Trimming the length of the Class II epitopes may improve immunogenicity, while trimming Class I epitopes from 21 to 15 amino acids did not affect immunogenicity. Further, immunogenicity to additional epitopes was detected in the 52 epitope constructs (FIG. 3). Both 52 mer and 20 mer constructs behaved comparably when re-stimulated with Class II epitopes (FIG. 4).

TABLE 3
Selected Sequences

| SEQ | |
|---|---|
| ID | |
| NO: | SEQUENCE |
| 1 | MPHSSLHPSIPCPRGHGAQKAALVLLSACLVTLWGLGEPPEHTLRYLVLHLASLQLGLLLNGVCSLAEELRHIHS |
| | RYRGSYWRTVRACLGCPLRRGALLLLSIYFYYSLPNAVGPPFTWMLALLGLSQALNILLGLKGLAPAEISAVCEKGNFN |
| | MANGLAWSYYIGYLRLILPELQARIRTYNQHNNLLRGAVSQRLYILLPLDCGVPDNLSMADPNIRFLDKLPQQTGD |
| | HAGIKDRVYSNSIYELLENGQRAGTCVLEYATPLQTLFAMSQYSQAGFSREDRLEQAKLFCRTLEDILADAPESCINNC |
| | RLIAYQEPADDSSFSLSQEVLRHLRQEEKEEVTVGSLKTSAVPSTSTMSQEPELLISGMEKPLPLRTDFS |
| | (huSTING (V155M); no epitope tag) |
| 2 | MPHSSLHPSIPCPRGHGAQKAALVLLSACLVTLWGLGEPPEHTLRYLVLHLASLQLGLLLNGVCSLAEELRHIHS |
| | RYRGSYWRTVRACLGCPLRRGALLLLSIYFYYSLPNAVGPPFTWMLALLGLSQALNILLGLKGLAPAEISAVCEKGNFN |
| | VAHGLAWSYYIGYLRLILPELQARIRTYNQHYNNLLRGAVSQRLYILLPLDCGVPDNLSMADPNIRFLDKLPQQTGDH |
| | AGIKDRVYSNSIYELLENGQRAGTCVLEYATPLQTLFAMSQYSQAGFSREDtLEQAKLFCRTLEDILADAPESQNNCRL |
| | IAYQEPADDSSFSLSQEVLRHLRQEEKEEVTVGSLKTSAVPSTSTMSQEPELLISGMEKPLPLRTDFS |
| | (Hu STING (R284T); no epitope tag) |
| 3 | MPHSSLHPSIPCPRGHGQKAALVLLSACLVTLWGLGEPPEHTLRYLVLHLASLQLGLLLNGVCSLAEELRHIHS |
| | RYRGSYWRTVRACLGCPLRRGALLLLSIYFYYSLPNAVGPPFTWMLALLGLSQALNILLGLKGLAPAEISAVCEKGNFN |
| | VAHGLAWSYYIGYLRLILPELQARIRTYNQHYNNLLRGAVSQRLYILLPLDCGVPDNLSMADPNIRFLDKLPQQTGDH |
| | AGIKDRVYSNSIYELLENGQRAGTCVLEYATPLQTLFAMSQYSQAGFSREDmLEQAKLFCRTLEDILADAPESQNNCR |
| | LIAYQEPADDSSFSLSQEVLRHLRQEEKEEVTVGSLKTSAVPSTSTMSQEPELLISGMEKPLPLRTDFS |
| | (hu STING (R284M); no epitope tag) |
| 4 | MPHSSLHPSIPCPRGHGAQKAALVLLSACLVTLWGLGEPPEHTLRYLVLHLASLCILGLLLNGVCSLAEELRHIHS |
| | RYRGSYWRTVRACLGCPLRRGALLLLSIYFYYSLPNAVGPPFTWMLALLGLSQALNILLGLKGLAPAEISAVCEKGNFN |
| | VAHGLAWSYYIGYLRLILPELQARIRTYNQHYNNLLRGAVSQRLYILLPLDCGVPDNLSMADPNIRFLDKLPQQTGDH |
| | AGIKDRVYSNSIYELLENGQRAGTCVLEYATPLQTLFAMSQYSQAGFSREDkLEQAKLFCRTLEDILADAPESQNNCR |
| | LIAYQEPADDSSFSLSQEVLRHLRQEEKEEVTVGSLKTSAVPSTSTMSQEPELLISGMEKPLPLRTDFS |
| | (Hu STING (R284K); no epitope tag) |
| 5 | MPHSSLHPSIPCPRGHGAQKAALVLLSACLVTLWGLGEPPEHTLRYLVLHLASLQLGLLLNGVCSLAEELRHIHS |
| | RYRGSYWRTVRACLGCPLRRGALLLLSIYFYYSLPNAVGPPFTWMLALLGLSQALNILLGLKGLAPAEISAVCEKGNFs |
| | VAHGLAWSYYIGYLRLILPELQARIRTYNQHYNNLLRGAVSQRLYILLPLDCGVPDNLSMADPNIRFLDKLPQQTGDH |
| | AGIKDRVYSNSIYELLENGQRAGTCVLEYATPLCITLFAMSQYSQAGFSREDRLEQAKLFCRTLEDILADAPESQNNCR |
| | LIAYQEPADDSSFSLSQEVLRHLRQEEKEEVTVGSLKTSAVPSTSTMSQEPELLISGMEKPLPLRTDFS |
| | (Hu STING (N154S); no epitope tag) |
| 6 | MPHSSLHPSIPCPRGHGAQKAALVLLSACLVTLWGLGEPPEHTLRYLVLHLASLQLGLLLNGVCSLAEELRHIHS |
| | RYRGSYWRTVRACLGCPLRRGALLLLSIYFYYSLPNAVGPPFTWMLALLGLSQALNILLGLKGLAPAEISAICEKGNFN |
| | VAHGLAWSYYIGYLRLILPELQARIRTYNQHYNNLLRGAVSQRLYILLPLDCGVPDNLSMADPNIRFLDKLPQQTGDH |
| | AGIKDRVYSNSIYELLENGQRAGTCVLEYATPLQTLFAMSQYSQAGFSREDRLEQAKLFCRTLEDILADAPESQNNCR |
| | LIAYQEPADDSSFSLSQEVLRHLRQEEKEEVTVGSLKTSAVPSTSTMSQEPELLISGMEKPLPLRTDFS |
| | (Hu STING (V147L); no epitope tag) |
| 7 | MPHSSLHPSIPCPRGHGAQKAALVLLSACLVTLWGLGEPPEHTLRYLVLHLASLQLGLLLNGVCSLAEELRHIHS |
| | RYRGSYWRTVRACLGCPLRRGALLLLSIYFYYSLPNAVGPPFTWMLALLGLSQALNILLGLKGLAPAEISAVCEKGNFN |
| | VAHGLAWSYYIGYLRLILPELQARIRTYNQHYNNLLRGAVSQRLYILLPLDCGVPDNLSMADPNIRFLDKLPQQTGDH |
| | AGIKDRVYSNSIYELLENGQRAGTCVLEYATPLQTLFAMSQYSQAGFSREDRLEQAKLFCRTLEDILADAPESQNNCR |
| | LIAYQqPADDSSFSLSQEVLRHLRQEEKEEVTVGSLKTSAVPSTSTMSQEPELLISGMEKPLPLRTDFS |
| | (Hu STING (E315Q); no epitope tag) |
| 8 | MPHSSLHPSIPCPRGHGAQKAALVLLSACLVTLWGLGEPPEHTLRYLVLHLASLQLGLLLNGVCSLAEELRHIHS |
| | RYRGSYWRTVRACLGCPLRRGALLLLSIYFYYSLPNAVGPPFTWMLALLGLSC1ALNILLGLKGLAPAEISAVCEKGNFN |
| | VAHGLAWSYYIGYLRLILPELQARIRTYNQHYNNLLRGAVSQRLYILLPLDCGVPDNLSMADPNIRFLDKLPQQTGDH |
| | AGIKDRVYSNSIYELLENGQRAGTCVLEYATPLQTLFAMSQYSQAGFSREDRLEQAKLFCRTLEDILADAPESQNNCR |
| | LIAYQEPADDSSFSLSQEVLRHLRQEEKEEVTVGSLKTSAVPSTSTMSQEPELLISGMEKPLPLaTDFS |
| | (Hu STING (R375A); no epitope tag) |
| 9 | MPHSSLHPSIPCPRGHGAQKAALVLLSACLVTLWGLGEPPEHTLRYLVLHLASLQLGLLLNGVCSLAEELRHIHS |
| | RYRGSYWRTVRACLGCPLRRGALLLLSIYFYYSLPNAVGPPFTWMLALLGLSQALNILLGLKGLAPAEISALCEKGNFS |
| | MAHGLAWSYYIGYLRLILPELQARIRTYNQHYNNLLRGAVSQRLYILLPLDCGVPDNLSMADPNIRFLDKLPQQTGD |
| | HAGIKDRVYSNSIYELLENGQRAGTCVLEYATPLQTLFAMSQYSQAGFSREDRLEQAKLFCRTLEDILADAPESQNNC |
| | RLIAYQEPADDSSFSLSQEVLRHLRQEEKEEVTVGSLKTSAVPSTSTMSQEPELLISGMEKPLPLRTDFS |
| | (Hu STING (V147L/N154S/V155M); no epitope tag) |
| 10 | MPHSSLHPSIPCPRGHGAQKAALVLLSACLVTLWGLGEPPEHTLRYLVLHLASLQLGLLLNGVCSLAEELRHIHS |
| | RYRGSYWRTVRACLGCPLRRGALLLLSIYFYYSLPNAVGPPFTWMLALLGLSQALNILLGLKGLAPAEISALCEKGNFS |
| | MAHGLAWSYYIGYLRLILPELQARIRTYNQHYNNLLRGAVSQRLYILLPLDCGVPDNLSMADPNIRFLDKLPQQTGD |
| | HAGIKDRVYSNSIYELLENGQRAGTCVLEYATPLQTLFAMSQYSQAGFSREDMLEQAKLFCRTLEDILADAPESQNN |
| | CRLIAYQEPADDSSFSLSQEVLRHLRQEEKEEVTVGSLKTSAVPSTSTMSQEPELLISGMEKPLPLRTDFS |
| | (Hu STING (R284M/V147L/N154S/V155M); no epitope tag) |
| 199 | ATGCCCCACAGTAGCCTCCACCCCAGCATCCCCTGCCCCAGAGGCCACGGCGCACAGAAGGCCGCCCTGG |
| | TGCTGCTGAGCGCCTGTCTGGTGACCCTGTGGGGTCTGGGCGAGCCCCCCGAGCACACCCTGCGGTACCTCGT |
| | GCTGCATCTGGCCAGCCTGCAGCTGGGCCTGCTGCTGAACGGCGTGTGCAGCCTGGCCGAAGAGCTGAGACA |
| | CATCCACAGCAGATACAGAGGCTCCTACTGGAGAACCGTCAGAGCCTGCCTCGGCTGTCCCCTGAGAAGAGGC |
| | GCCCTGCTGCTCCTGAGCATCTACTTCTACTACAGCCTGCCCAACGCCGTGGGCCCCCCCTTCACCTGGATGCTG |
| | GCCCTGCTGGGCCTGAGCCAGGCCCTGAACATCCTGCTGGGCCTGAAGGGCTTGGCCCCCGCCGAGATCTCCG |
| | CCGTGTGCGAGAAGGGCAACTTCAACATGGCCCATGGCCTTGCCTGGTCCTACTACATCGGCTACCTGAGACTG |
| | ATCCTGCCCGAGCTGCAGGCCAGAATCAGAACCTACAACCAGCACTACAACAACCTGCTGAGAGGCGCCGTGA |
| | GCCAAAGACTGTACATCCTGCTGCCCCTGGACTGCGGCGTGCCCGACAACCTTAGCATGGCCGACCCCAACATC |
| | AGATTCCTGGACAAGCTGCCCCAGCAGACCGGCGACCACGCCGGCATCAAGGACAGAGTGTACAGCAACAGC |
| | ATCTACGAGCTGCTGGAGAACGGCCAGAGAGCCGGCACCTGCGTGCTGGAGTACGCCACCCCCCTGCAGACCC |
| | TGTTCGCCATGAGCCAGTACAGCCAGGCCGGCTTCAGCAGAGAGGACAGACTGGAGCAAGCCAAGCTGTTCTG |
| | CAGAACCCTGGAGGACATCCTGGCGGACGCCCCCGAGAGCCAAAACAACTGCAGACTGATCGCCTACCAGGA |
| | GCCCGCCGACGACAGCAGCTTCAGCCTGAGCCAGGAAGTGCTGAGACACCTGAGACAGGAAGAGAAGGAGG |
| | AGGTGACCGTGGGAAGCCTGAAGACCAGCGCCGTGCCCAGCACCAGCACCATGAGCCAGGAGCCCGAGCTGC |
| | TGATCAGCGGCATGGAGAAGCCCCTGCCCCTGAGAACCGACTTCAGC |
| | (huSTING (V155M); no epitope tag; nucleotide sequence) |
| 200 | ATGCCTCACAGCAGCCTGCACCCTAGCATCCCTTGCCCTAGAGGCCACGGCGCCCAGAAGGCCGCCCTGG |
| | TGCTGCTGAGCGCCTGCCTGGTGACCCTGTGGGGCCTGGGCGAGCCTCCTGAGCACACCCTGAGATACCTGGT |
| | GCTGCACCTGGCCAGCCTGCAGCTGGGCCTGCTGCTGAACGGCGTGTGCAGCCTGGCCGAGGAGCTGAGACA |
| | CATCCACAGCAGATACAGAGGCAGCTACTGGAGAACCGTGAGAGCCTGCCTGGGCTGCCCTCTGAGAAGAGG |
| | CGCCCTGCTGCTGCTGAGCATCTACTTCTACTACAGCCTGCCTAACGCCGTGGGCCCTCCTTTCACCTGGATGCT |
| | GGCCCTGCTGGGCCTGAGCCAGGCCCTGAACATCCTGCTGGGCCTGAAGGGCCTGGCCCCTGCCGAGATCAGC |
| | GCCGTGTGCGAGAAGGGCAACTTCAACGTGGCCCACGGCCTGGCCTGGAGCTACTACATCGGCTACCTGAGAC |
| | TGATCCTGCCTGAGCTGCAGGCCAGAATCAGAACCTACAACCAGCACTACAACAACCTGCTGAGAGGCGCCGT |
| | GAGCCAGAGACTGTACATCCTGCTGCCTCTGGACTGCGGCGTGCCTGACAACCTGAGCATGGCCGACCCTAAC |
| | ATCAGATTCCTGGACAAGCTGCCTCAGCAGACCGGCGACCACGCCGGCATCAAGGACAGAGTGTACAGCAACA |
| | GCATCTACGAGCTGCTGGAGAACGGCCAGAGAGCCGGCACCTGCGTGCTGGAGTACGCCACCCCTCTGCAGAC |
| | CCTGTTCGCCATGAGCCAGTACAGCCAGGCCGGCTTCAGCAGAGAGGACACCCTGGAGCAGGCCAAGCTGTTC |
| | TGCAGAACCCTGGAGGACATCCTGGCCGACGCCCCTGAGAGCCAGAACAACTGCAGACTGATCGCCTACCAGG |
| | AGCCTGCCGACGACAGCAGCTTCAGCCTGAGCCAGGAGGTGCTGAGACACCTGAGACAGGAGGAGAAGGAG |
| | GAGGTGACCGTGGGCAGCCTGAAGACCAGCGCCGTGCCTAGCACCAGCACCATGAGCCAGGAGCCTGAGCTG |
| | CTGATCAGCGGCATGGAGAAGCCTCTGCCTCTGAGAACCGACTTCAGC |
| | (Hu STING (R284T); no epitope tag; nucleotide sequence) |
| 201 | ATGCCCCACAGCAGCCTGCACCCCTCCATCCCCTGTCCCAGAGGCCACGGCGCCCAGAAGGCCGCCCTGG |
| | TGCTGCTGAGCGCCTGCCTGGTGACCTTATGGGGCCTGGGCGAGCCCCCCGAGCACACCCTGAGATACCTGGT |
| | CCTGCACCTGGCCAGCCTCCAGCTGGGCCTGCTGCTCAACGGCGTGTGTAGCCTGGCCGAGGAGCTGAGACAC |
| | ATCCACAGCAGATACAGAGGCAGCTACTGGAGAACCGTGAGAGCCTGCCTGGGTTGCCCACTGAGAAGAGGA |
| | GCTCTGCTGCTGCTGAGCATCTACTTCTACTACTCGCTGCCCAACGCTGTGGGCCCCCCCTTCACCTGGATGCTG |
| | GCCCTGCTGGGTCTGAGCCAGGCCCTGAACATCCTCCTGGGCCTGAAGGGCCTGGCCCCCGCCGAGATAAGCG |
| | CCGTTTGCGAGAAGGGCAACTTCAACGTGGCCCATGGCCTGGCCTGGAGCTACTACATCGGCTACTTACGCCTG |
| | ATCCTGCCCGAGCTGCAGGCCAGAATCAGAACCTACAACCAGCATTACAACAACCTGCTGAGAGGCGCCGTGA |
| | GCCAGAGACTGTATATCCTGCTGCCCCTGGACTGCGGCGTGCCCGACAACCTGAGCATGGCCGACCCCAACATC |
| | AGATTCCTGGACAAGCTCCCCCAGCAGACCGGCGACCACGCCGGAATCAAAGACAGAGTGTATAGCAACAGCA |
| | TCTACGAGCTGCTGGAGAACGGCCAGAGAGCCGGCACCTGCGTACTGGAGTACGCCACCCCCTTGCAGACCCT |
| | GTTTGCCATGAGCCAGTACAGCCAGGCCGGCTTCAGCAGAGAGGACATGCTGGAGCAGGCCAAGCTGTTCTGC |
| | AGAACCCTGGAGGACATCCTGGCCGACGCCCCCGAGAGCCAGAACAACTGCAGACTGATCGCCTACCAAGAGC |
| | CCGCCGACGACAGCAGCTTCAGCTTAAGCCAGGAGGTGCTGAGACATCTGAGACAGGAGGAGAAGGAGGAG |
| | GTGACCGTGGGCAGCCTCAAGACCAGCGCTGTGCCCTCTACCAGCACCATGAGCCAGGAGCCCGAGCTGCTGA |
| | TCAGCGGCATGGAGAAGCCCCTGCCCCTGAGAACAGACTTCAGC |
| | (huSTING (R284M); no epitope tag; nudeotide sequence) |
| 202 | ATGCCCCATAGCAGCCTGCACCCCAGCATCCCCTGCCCCAGAGGCCACGGCGCCCAGAAGGCCGCCCTGG |
| | TCCTGCTGAGCGCATGCCTGGTCACCCTGTGGGGCCTGGGCGAGCCCCCCGAGCACACCCTGAGATACCTGGT |
| | GCTGCACCTCGCCAGCCTGCAGCTGGGCCTGCTGCTGAACGGCGTGTGCAGCCTGGCCGAGGAGCTGAGACA |
| | CATCCACAGCAGATATAGAGGCAGCTACTGGAGAACCGTGAGAGCTTGCCTCGGCTGCCCCCTGAGAAGAGGC |
| | GCCCTGCTGCTGCTGAGCATCTACTTTTACTACAGCCTGCCCAACGCTGTGGGCCCCCCTTTCACGTGGATGCTC |
| | GCCCTGCTGGGACTGAGCCAGGCCCTGAACATCCTGCTGGGCCTTAAGGGCCTAGCCCCCGCCGAGATCAGCG |
| | CCGTGTGCGAGAAGGGCAACTTCAATGTGGCCCACGGCCTGGCCTGGAGCTACTACATCGGCTACCTGAGACT |
| | GATCCTGCCCGAGCTGCAGGCCAGAATCAGAACCTACAATCAGCACTACAACAACCTGCTGAGAGGCGCCGTG |
| | AGCCAGAGACTGTACATCCTGCTGCCCCTGGACTGCGGCGTGCCCGACAACCTCAGCATGGCCGACCCCAACAT |
| | CAGATTCCTGGACAAGCTGCCCCAGCAGACCGGCGACCACGCCGGCATCAAGGATCGCGTGTACAGCAACAGC |
| | ATCTACGAGCTGCTGGAAAACGGCCAGAGAGCCGGAACCTGCGTGCTGGAGTACGCCACACCCCTGCAGACCC |
| | TGTTCGCCATGAGCCAGTACAGCCAGGCCGGCTTCAGCAGAGAGGACAAGCTGGAGCAGGCCAAGCTGTTCT |
| | GCAGAACCCTGGAGGATATCCTCGCCGACGCCCCCGAGAGCCAGAACAACTGCAGGCTGATCGCGTACCAGG |
| | AGCCCGCTGACGACAGCAGCTTTAGCCTGAGCCAGGAGGTGCTGAGACATCTGCGTCAAGAGGAAAAGGAGG |
| | AGGTGACCGTGGGCTCCCTGAAGACCAGCGCCGTGCCCAGCACCAGCACCATGAGCCAGGAGCCCGAGCTGC |
| | TGATCAGCGGCATGGAGAAGCCACTGCCCCTCAGAACCGACTTCAGC |
| | (HuSTING (R284K; no epitope tag; nudeotide sequence) |
| 203 | ATGCCTCACAGCAGCCTGCACCCTAGCATCCCTTGCCCTAGAGGCCACGGCGCCCAGAAGGCCGCCCTGG |
| | TGCTGCTGAGCGCCTGCCTGGTGACCCTGTGGGGCCTGGGCGAGCCTCCTGAGCACACCCTGAGATACCTGGT |
| | GCTGCACCTGGCCAGCCTGCAGCTGGGCCTGCTGCTGAACGGCGTGTGCAGCCTGGCCGAGGAGCTGAGACA |
| | CATCCACAGCAGATACAGAGGCAGCTACTGGAGAACCGTGAGAGCCTGCCTGGGCTGCCCTCTGAGAAGAGG |
| | CGCCCTGCTGCTGCTGAGCATCTACTTCTACTACAGCCTGCCTAACGCCGTGGGCCCTCCTTTCACCTGGATGCT |
| | GGCCCTGCTGGGCCTGAGCCAGGCCCTGAACATCCTGCTGGGCCTGAAGGGCCTGGCCCCTGCCGAGATCAGC |
| | GCCGTGTGCGAGAAGGGCAACTTCAGCGTGGCCCACGGCCTGGCCTGGAGCTACTACATCGGCTACCTGAGAC |
| | TGATCCTGCCTGAGCTGCAGGCCAGAATCAGAACCTACAACCAGCACTACAACAACCTGCTGAGAGGCGCCGT |
| | GAGCCAGAGACTGTACATCCTGCTGCCTCTGGACTGCGGCGTGCCTGACAACCTGAGCATGGCCGACCCTAAC |
| | ATCAGATTCCTGGACAAGCTGCCTCAGCAGACCGGCGACCACGCCGGCATCAAGGACAGAGTGTACAGCAACA |
| | GCATCTACGAGCTGCTGGAGAACGGCCAGAGAGCCGGCACCTGCGTGCTGGAGTACGCCACCCCTCTGCAGAC |
| | CCTGTTCGCCATGAGCCAGTACAGCCAGGCCGGCTTCAGCAGAGAGGACAGACTGGAGCAGGCCAAGCTGTTC |
| | TGCAGAACCCTGGAGGACATCCTGGCCGACGCCCCTGAGAGCCAGAACAACTGCAGACTGATCGCCTACCAGG |
| | AGCCTGCCGACGACAGCAGCTTCAGCCTGAGCCAGGAGGTGCTGAGACACCTGAGACAGGAGGAGAAGGAG |
| | GAGGTGACCGTGGGCAGCCTGAAGACCAGCGCCGTGCCTAGCACCAGCACCATGAGCCAGGAGCCTGAGCTG |
| | CTGATCAGCGGCATGGAGAAGCCTCTGCCTCTGAGAACCGACTTCAGC |
| | (Hu STING (N154S); no epitope tag; nucleotide sequence) |
| 204 | ATGCCTCACAGCAGCCTGCACCCTAGCATCCCTTGCCCTAGAGGCCACGGCGCCCAGAAGGCCGCCCTGG |
| | TGCTGCTGAGCGCCTGCCTGGTGACCCTGTGGGGCCTGGGCGAGCCTCCTGAGCACACCCTGAGATACCTGGT |
| | GCTGCACCTGGCCAGCCTGCAGCTGGGCCTGCTGCTGAACGGCGTGTGCAGCCTGGCCGAGGAGCTGAGACA |
| | CATCCACAGCAGATACAGAGGCAGCTACTGGAGAACCGTGAGAGCCTGCCTGGGCTGCCCTCTGAGAAGAGG |
| | CGCCCTGCTGCTGCTGAGCATCTACTTCTACTACAGCCTGCCTAACGCCGTGGGCCCTCCTTTCACCTGGATGCT |
| | GGCCCTGCTGGGCCTGAGCCAGGCCCTGAACATCCTGCTGGGCCTGAAGGGCCTGGCCCCTGCCGAGATCAGC |
| | GCCCTGTGCGAGAAGGGCAACTTCAACGTGGCCCACGGCCTGGCCTGGAGCTACTACATCGGCTACCTGAGAC |
| | TGATCCTGCCTGAGCTGCAGGCCAGAATCAGAACCTACAACCAGCACTACAACAACCTGCTGAGAGGCGCCGT |
| | GAGCCAGAGACTGTACATCCTGCTGCCTCTGGACTGCGGCGTGCCTGACAACCTGAGCATGGCCGACCCTAAC |
| | ATCAGATTCCTGGACAAGCTGCCTCAGCAGACCGGCGACCACGCCGGCATCAAGGACAGAGTGTACAGCAACA |
| | GCATCTACGAGCTGCTGGAGAACGGCCAGAGAGCCGGCACCTGCGTGCTGGAGTACGCCACCCCTCTGCAGAC |
| | CCTGTTCGCCATGAGCCAGTACAGCCAGGCCGGCTTCAGCAGAGAGGACAGACTGGAGCAGGCCAAGCTGTTC |
| | TGCAGAACCCTGGAGGACATCCTGGCCGACGCCCCTGAGAGCCAGAACAACTGCAGACTGATCGCCTACCAGG |
| | AGCCTGCCGACGACAGCAGCTTCAGCCTGAGCCAGGAGGTGCTGAGACACCTGAGACAGGAGGAGAAGGAG |
| | GAGGTGACCGTGGGCAGCCTGAAGACCAGCGCCGTGCCTAGCACCAGCACCATGAGCCAGGAGCCTGAGCTG |
| | CTGATCAGCGGCATGGAGAAGCCTCTGCCTCTGAGAACCGACTTCAGC |
| | (Hu STING (V147L); no epitope tag; nucleotide sequence) |
| 205 | ATGCCTCACAGCAGCCTGCACCCTAGCATCCCTTGCCCTAGAGGCCACGGCGCCCAGAAGGCCGCCCTGG |
| | TGCTGCTGAGCGCCTGCCTGGTGACCCTGTGGGGCCTGGGCGAGCCTCCTGAGCACACCCTGAGATACCTGGT |
| | GCTGCACCTGGCCAGCCTGCAGCTGGGCCTGCTGCTGAACGGCGTGTGCAGCCTGGCCGAGGAGCTGAGACA |
| | CATCCACAGCAGATACAGAGGCAGCTACTGGAGAACCGTGAGAGCCTGCCTGGGCTGCCCTCTGAGAAGAGG |
| | CGCCCTGCTGCTGCTGAGCATCTACTTCTACTACAGCCTGCCTAACGCCGTGGGCCCTCCTTTCACCTGGATGCT |
| | GGCCCTGCTGGGCCTGAGCCAGGCCCTGAACATCCTGCTGGGCCTGAAGGGCCTGGCCCCTGCCGAGATCAGC |
| | GCCGTGTGCGAGAAGGGCAACTTCAACGTGGCCCACGGCCTGGCCTGGAGCTACTACATCGGCTACCTGAGAC |
| | TGATCCTGCCTGAGCTGCAGGCCAGAATCAGAACCTACAACCAGCACTACAACAACCTGCTGAGAGGCGCCGT |
| | GAGCCAGAGACTGTACATCCTGCTGCCTCTGGACTGCGGCGTGCCTGACAACCTGAGCATGGCCGACCCTAAC |
| | ATCAGATTCCTGGACAAGCTGCCTCAGCAGACCGGCGACCACGCCGGCATCAAGGACAGAGTGTACAGCAACA |
| | GCATCTACGAGCTGCTGGAGAACGGCCAGAGAGCCGGCACCTGCGTGCTGGAGTACGCCACCCCTCTGCAGAC |
| | CCTGTTCGCCATGAGCCAGTACAGCCAGGCCGGCTTCAGCAGAGAGGACAGACTGGAGCAGGCCAAGCTGTTC |
| | TGCAGAACCCTGGAGGACATCCTGGCCGACGCCCCTGAGAGCCAGAACAACTGCAGACTGATCGCCTACCAGC |
| | AGCCTGCCGACGACAGCAGCTTCAGCCTGAGCCAGGAGGTGCTGAGACACCTGAGACAGGAGGAGAAGGAG |
| | GAGGTGACCGTGGGCAGCCTGAAGACCAGCGCCGTGCCTAGCACCAGCACCATGAGCCAGGAGCCTGAGCTG |
| | CTGATCAGCGGCATGGAGAAGCCTCTGCCTCTGAGAACCGACTTCAGC |
| | (Hu STING (E315Q); no epitope tag; nucleotide sequence) |
| 206 | ATGCCTCACAGCAGCCTGCACCCTAGCATCCCTTGCCCTAGAGGCCACGGCGCCCAGAAGGCCGCCCTGG |
| | TGCTGCTGAGCGCCTGCCTGGTGACCCTGTGGGGCCTGGGCGAGCCTCCTGAGCACACCCTGAGATACCTGGT |
| | GCTGCACCTGGCCAGCCTGCAGCTGGGCCTGCTGCTGAACGGCGTGTGCAGCCTGGCCGAGGAGCTGAGACA |
| | CATCCACAGCAGATACAGAGGCAGCTACTGGAGAACCGTGAGAGCCTGCCTGGGCTGCCCTCTGAGAAGAGG |
| | CGCCCTGCTGCTGCTGAGCATCTACTTCTACTACAGCCTGCCTAACGCCGTGGGCCCTCCTTTCACCTGGATGCT |
| | GGCCCTGCTGGGCCTGAGCCAGGCCCTGAACATCCTGCTGGGCCTGAAGGGCCTGGCCCCTGCCGAGATCAGC |
| | GCCGTGTGCGAGAAGGGCAACTTCAACGTGGCCCACGGCCTGGCCTGGAGCTACTACATCGGCTACCTGAGAC |
| | TGATCCTGCCTGAGCTGCAGGCCAGAATCAGAACCTACAACCAGCACTACAACAACCTGCTGAGAGGCGCCGT |
| | GAGCCAGAGACTGTACATCCTGCTGCCTCTGGACTGCGGCGTGCCTGACAACCTGAGCATGGCCGACCCTAAC |
| | ATCAGATTCCTGGACAAGCTGCCTCAGCAGACCGGCGACCACGCCGGCATCAAGGACAGAGTGTACAGCAACA |
| | GCATCTACGAGCTGCTGGAGAACGGCCAGAGAGCCGGCACCTGCGTGCTGGAGTACGCCACCCCTCTGCAGAC |
| | CCTGTTCGCCATGAGCCAGTACAGCCAGGCCGGCTTCAGCAGAGAGGACAGACTGGAGCAGGCCAAGCTGTTC |
| | TGCAGAACCCTGGAGGACATCCTGGCCGACGCCCCTGAGAGCCAGAACAACTGCAGACTGATCGCCTACCAGG |
| | AGCCTGCCGACGACAGCAGCTTCAGCCTGAGCCAGGAGGTGCTGAGACACCTGAGACAGGAGGAGAAGGAG |
| | GAGGTGACCGTGGGCAGCCTGAAGACCAGCGCCGTGCCTAGCACCAGCACCATGAGCCAGGAGCCTGAGCTG |
| | CTGATCAGCGGCATGGAGAAGCCTCTGCCTCTGGCCACCGACTTCAGC |
| | (Hu STING (R375A); no epitope tag; nucleotide sequence) |
| 207 | ATGCCTCACAGCAGCCTGCACCCTAGCATCCCTTGCCCTAGAGGCCACGGCGCCCAGAAGGCCGCCCTGG |
| | TGCTGCTGAGCGCCTGCCTGGTGACCCTGTGGGGCCTGGGCGAGCCTCCTGAGCACACCCTGAGATACCTGGT |
| | GCTGCACCTGGCCAGCCTGCAGCTGGGCCTGCTGCTGAACGGCGTGTGCAGCCTGGCCGAGGAGCTGAGACA |
| | CATCCACAGCAGATACAGAGGCAGCTACTGGAGAACCGTGAGAGCCTGCCTGGGCTGCCCTCTGAGAAGAGG |
| | CGCCCTGCTGCTGCTGAGCATCTACTTCTACTACAGCCTGCCTAACGCCGTGGGCCCTCCTTTCACCTGGATGCT |
| | GGCCCTGCTGGGCCTGAGCCAGGCCCTGAACATCCTGCTGGGCCTGAAGGGCCTGGCCCCTGCCGAGATCAGC |
| | GCCCTGTGCGAGAAGGGCAACTTCAGCATGGCCCACGGCCTGGCCTGGAGCTACTACATCGGCTACCTGAGAC |
| | TGATCCTGCCTGAGCTGCAGGCCAGAATCAGAACCTACAACCAGCACTACAACAACCTGCTGAGAGGCGCCGT |
| | GAGCCAGAGACTGTACATCCTGCTGCCTCTGGACTGCGGCGTGCCTGACAACCTGAGCATGGCCGACCCTAAC |
| | ATCAGATTCCTGGACAAGCTGCCTCAGCAGACCGGCGACCACGCCGGCATCAAGGACAGAGTGTACAGCAACA |
| | GCATCTACGAGCTGCTGGAGAACGGCCAGAGAGCCGGCACCTGCGTGCTGGAGTACGCCACCCCTCTGCAGAC |
| | CCTGTTCGCCATGAGCCAGTACAGCCAGGCCGGCTTCAGCAGAGAGGACAGACTGGAGCAGGCCAAGCTGTTC |
| | TGCAGAACCCTGGAGGACATCCTGGCCGACGCCCCTGAGAGCCAGAACAACTGCAGACTGATCGCCTACCAGG |
| | AGCCTGCCGACGACAGCAGCTTCAGCCTGAGCCAGGAGGTGCTGAGACACCTGAGACAGGAGGAGAAGGAG |
| | GAGGTGACCGTGGGCAGCCTGAAGACCAGCGCCGTGCCTAGCACCAGCACCATGAGCCAGGAGCCTGAGCTG |
| | CTGATCAGCGGCATGGAGAAGCCTCTGCCTCTGAGAACCGACTTCAGC |
| | (Hu STING (V147L/N154S/V155M); no epitope tag; nucleotide sequence) |
| 208 | ATGCCTCACAGCAGCCTGCACCCTAGCATCCCTTGCCCTAGAGGCCACGGCGCCCAGAAGGCCGCCCTGG |
| | TGCTGCTGAGCGCCTGCCTGGTGACCCTGTGGGGCCTGGGCGAGCCTCCTGAGCACACCCTGAGATACCTGGT |
| | GCTGCACCTGGCCAGCCTGCAGCTGGGCCTGCTGCTGAACGGCGTGTGCAGCCTGGCCGAGGAGCTGAGACA |
| | CATCCACAGCAGATACAGAGGCAGCTACTGGAGAACCGTGAGAGCCTGCCTGGGCTGCCCTCTGAGAAGAGG |
| | CGCCCTGCTGCTGCTGAGCATCTACTTCTACTACAGCCTGCCTAACGCCGTGGGCCCTCCTTTCACCTGGATGCT |
| | GGCCCTGCTGGGCCTGAGCCAGGCCCTGAACATCCTGCTGGGCCTGAAGGGCCTGGCCCCTGCCGAGATCAGC |
| | GCCCTGTGCGAGAAGGGCAACTTCAGCATGGCCCACGGCCTGGCCTGGAGCTACTACATCGGCTACCTGAGAC |
| | TGATCCTGCCTGAGCTGCAGGCCAGAATCAGAACCTACAACCAGCACTACAACAACCTGCTGAGAGGCGCCGT |
| | GAGCCAGAGACTGTACATCCTGCTGCCTCTGGACTGCGGCGTGCCTGACAACCTGAGCATGGCCGACCCTAAC |
| | ATCAGATTCCTGGACAAGCTGCCTCAGCAGACCGGCGACCACGCCGGCATCAAGGACAGAGTGTACAGCAACA |
| | GCATCTACGAGCTGCTGGAGAACGGCCAGAGAGCCGGCACCTGCGTGCTGGAGTACGCCACCCCTCTGCAGAC |
| | CCTGTTCGCCATGAGCCAGTACAGCCAGGCCGGCTTCAGCAGAGAGGACATGCTGGAGCAGGCCAAGCTGTTC |
| | TGCAGAACCCTGGAGGACATCCTGGCCGACGCCCCTGAGAGCCAGAACAACTGCAGACTGATCGCCTACCAGG |
| | AGCCTGCCGACGACAGCAGCTTCAGCCTGAGCCAGGAGGTGCTGAGACACCTGAGACAGGAGGAGAAGGAG |
| | GAGGTGACCGTGGGCAGCCTGAAGACCAGCGCCGTGCCTAGCACCAGCACCATGAGCCAGGAGCCTGAGCTG |
| | CTGATCAGCGGCATGGAGAAGCCTCTGCCTCTGAGAACCGACTTCAGC |
| | (Hu STING (R284M/V147L/N154S/V155M); no epitope tag; nucleotide sequence) |
| 209 | TGATAATAGGCTGGAGCCTCGGTGGCCTAGCTTCTTGCCCCTTGGGCCTCCCCCCAGCCCCTCCTCCCCTTC |
| | CTGCACCCGTACCCCCCAAACACCATTGTCACACTCCAGTGGTCTTTGAATAAAGTCTGAGTGGGCGGC |
| | (3′ UTR used in STING V155M construct, containing miR122 binding site) |
| 224 | MPHSSLHPSIPCPRGHGAQKAALVLLSACLVTLWGLGEPPEHTLRYLVLHLASLQLGLLLNGVCSLAEELRHIHS |
| | RYRGSYWRTVRACLGCPLRRGALLLLSIYFYYSLPNAVGPPFTWMLALLGLSQALNILLGLKGLAPAEISAVCEKGNFN |
| | VAHGLAWSYYIGYLRLILPELQARIRTYNQHYNNLLRGAVSQRLYILLPLDCGVPDNLSMADPNIRFLDKLPQQTGDH |
| | AGIKDRVYSNSIYELLENGQRAGTCVLEYATPLQTLFAMSQYSQAGFSREDkLEQAKLFCRTLEDILADAPESQNNCR |
| | LIAMEPADDSSFSLSQEVLRHLRQEEKEEVTVGSLKTSAVPSTSTMSQEPELLISGMEKPLPLRTDFST |
| | (Hu STING (R284K) var; no epitope tag) |
| 225 | ATGCCCCATAGCAGCCTGCACCCCAGCATCCCCTGCCCCAGAGGCCACGGCGCCCAGAAGGCCGCCCTGG |
| | TCCTGCTGAGCGCATGCCTGGTCACCCTGTGGGGCCTGGGCGAGCCCCCCGAGCACACCCTGAGATACCTGGT |
| | GCTGCACCTCGCCAGCCTGCAGCTGGGCCTGCTGCTGAACGGCGTGTGCAGCCTGGCCGAGGAGCTGAGACA |
| | CATCCACAGCAGATATAGAGGCAGCTACTGGAGAACCGTGAGAGCTTGCCTCGGCTGCCCCCTGAGAAGAGGC |
| | GCCCTGCTGCTGCTGAGCATCTACTTTTACTACAGCCTGCCCAACGCTGTGGGCCCCCCTTTCACGTGGATGCTC |
| | GCCCTGCTGGGACTGAGCCAGGCCCTGAACATCCTGCTGGGCCTTAAGGGCCTAGCCCCCGCCGAGATCAGCG |
| | CCGTGTGCGAGAAGGGCAACTTCAATGTGGCCCACGGCCTGGCCTGGAGCTACTACATCGGCTACCTGAGACT |
| | GATCCTGCCCGAGCTGCAGGCCAGAATCAGAACCTACAATCAGCACTACAACAACCTGCTGAGAGGCGCCGTG |
| | AGCCAGAGACTGTACATCCTGCTGCCCCTGGACTGCGGCGTGCCCGACAACCTCAGCATGGCCGACCCCAACAT |
| | CAGATTCCTGGACAAGCTGCCCCAGCAGACCGGCGACCACGCCGGCATCAAGGATCGCGTGTACAGCAACAGC |
| | ATCTACGAGCTGCTGGAAAACGGCCAGAGAGCCGGAACCTGCGTGCTGGAGTACGCCACACCCCTGCAGACCC |
| | TGTTCGCCATGAGCCAGTACAGCCAGGCCGGCTTCAGCAGAGAGGACAAGCTGGAGCAGGCCAAGCTGTTCT |
| | GCAGAACCCTGGAGGATATCCTCGCCGACGCCCCCGAGAGCCAGAACAACTGCAGGCTGATCGCGTACCAGG |
| | AGCCCGCTGACGACAGCAGCTTTAGCCTGAGCCAGGAGGTGCTGAGACATCTGCGTCAAGAGGAAAAGGAGG |
| | AGGTGACCGTGGGCTCCCTGAAGACCAGCGCCGTGCCCAGCACCAGCACCATGAGCCAGGAGCCCGAGCTGC |
| | TGATCAGCGGCATGGAGAAGCCACTGCCCCTCAGAACCGACTTCAGCACC |
| | (Hu STING (R284K) var; no epitope tag) |

### Example 7. Activating Oncogene KRAS Mutations

KRAS is the most frequently mutated oncogene in human cancer (−15%). KRAS mutations are mostly conserved in a single “hotspot”, and activate the oncogene. Prior research has shown limited ability to raise T cells specific to the oncogenic mutation. However, much of this was done in the context of the most common HLA allele (A2, which occurs in ˜50% of Caucasians). More recently, it has been demonstrated that (a) specific T cells can be generated against point mutations in the context of less common HLA alleles (A1, C8), and (b) growing these cells ex-vivo and transferring them back to the patient has mediated a dramatic tumor response in a patient with lung cancer. (N Engl J Med 2016; 375:2255-2262 Dec. 8, 2016DOI: 10.1056/NEJMoa1609279).

As shown in Table 4 below, in CRC (colorectal cancer), only 3 mutations (G12V, G12D, and G13D) account for 96% of cases. Furthermore, all CRC patients get typed for KRAS mutations as standard of care.

TABLE 4
COSMIC* case counts

| | All cancers | % | CRC | % |
|---|---|---|---|---|
| G12S | 1849 | 1% | | |
| G12V | 9213 | 4% | 5215 | 29% |
| G12C | 4535 | 2% | | |
| G12D | 13634 | 7% | 8083 | 44% |
| G12A | 2179 | 1% | | |
| G12R | 1244 | 1% | | |
| G13D | 5084 | 2% | 4267 | 23% |
| | | 18% | | 96% |
| Tested | 208629 | | 18271 | |

*http://cancer.sanger.ac.uk/cosmic/gene/analysis?In=KRAS

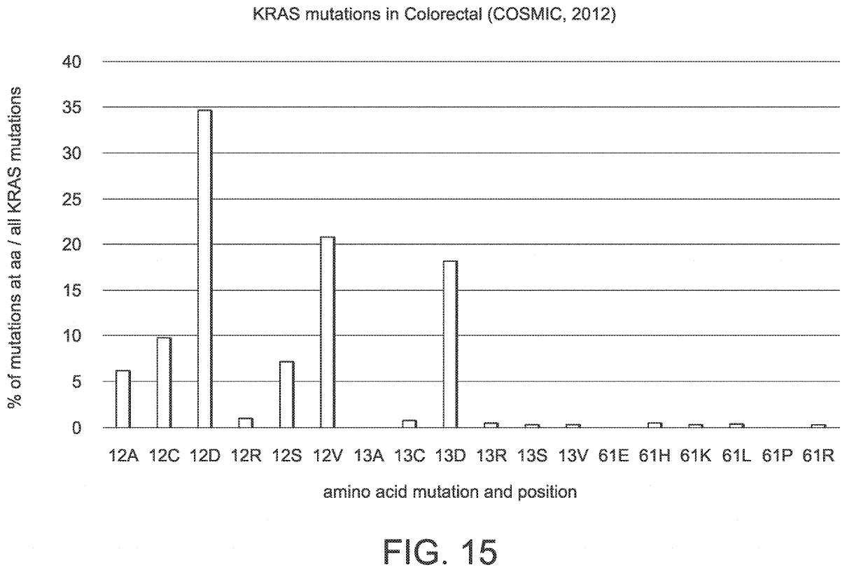

In another COSMIC data set, 73.68% of KRAS mutations in colorectal cancer are accounted for by these 3 mutations (G12V, G12D, and G13D) (FIG. 15 and Table 5).

TABLE 5

| | colon | % | rectal | % | total | % |
|---|---|---|---|---|---|---|
| 12D | 635 | 35.04 | 178 | 33.46 | 813 | 34.68 |
| 12V | 364 | 20.09 | 124 | 23.31 | 488 | 20.82 |
| 13D | 338 | 18.65 | 88 | 16.54 | 426 | 18.17 |
| | | | | | | 73.68 |

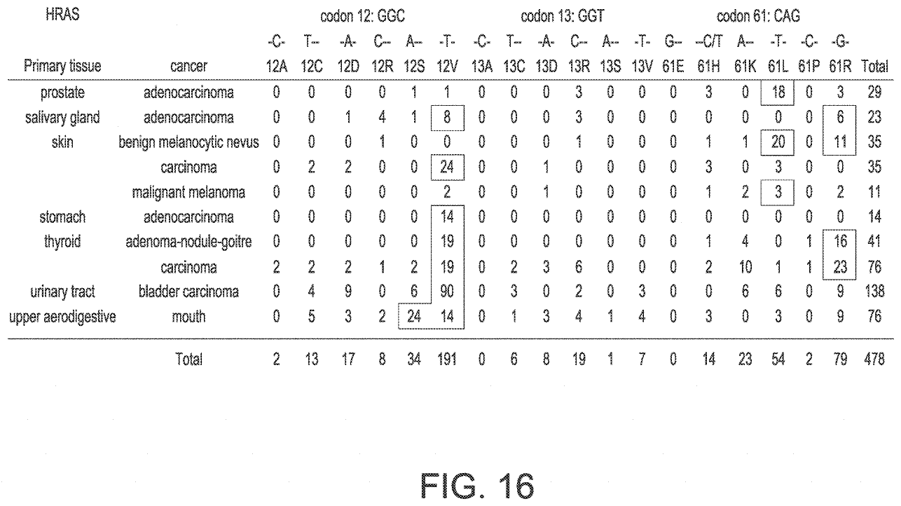

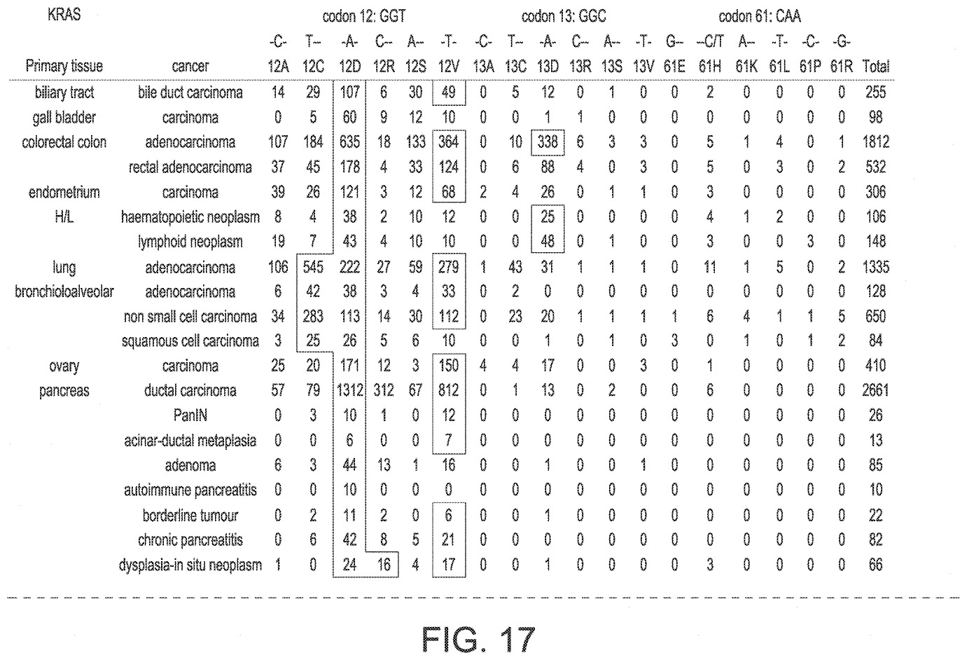

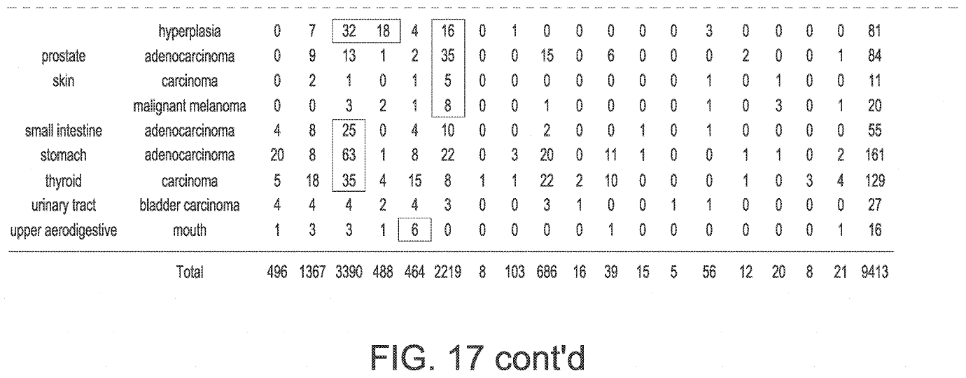

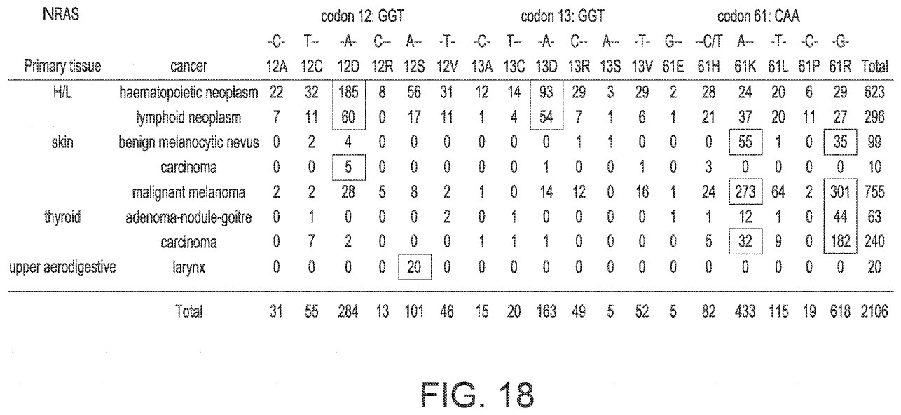

FIGS. 16, 17, and 18 depict isoform-specific point mutation specificity for HRAS, KRAS, and NRAS, respectively. Data representing total number of tumors with each point mutation were collated from COSMIC v52 release. Single base mutations generating each amino acid substitution are indicated. The most frequent mutations for each isoform for each cancer type are highlighted with grey shading. H/L: hematopoietic/lymphoid tissues. (Prior et al. Cancer Res. 2012 May 15; 72(10): 2457-2467).

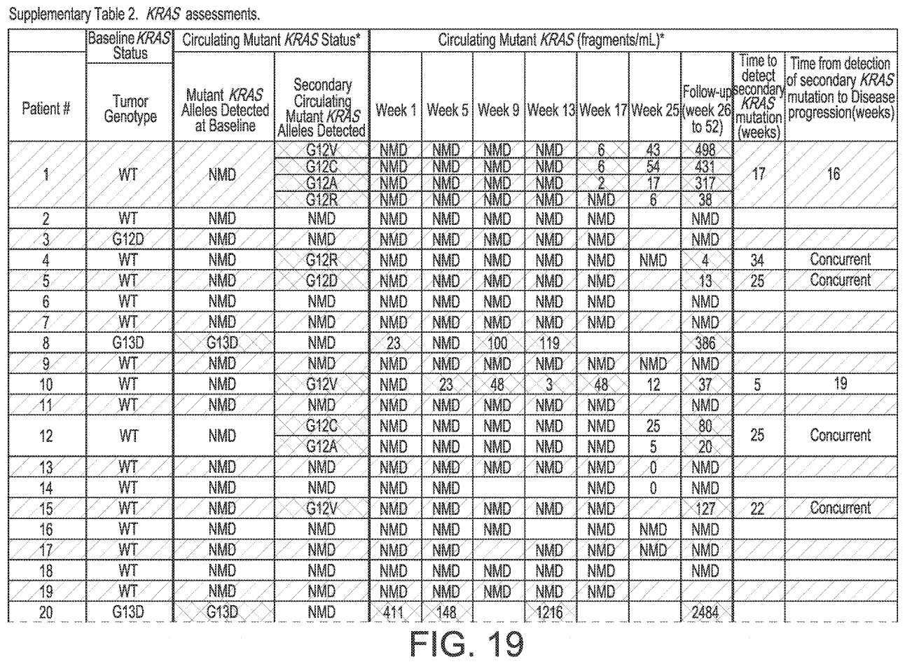

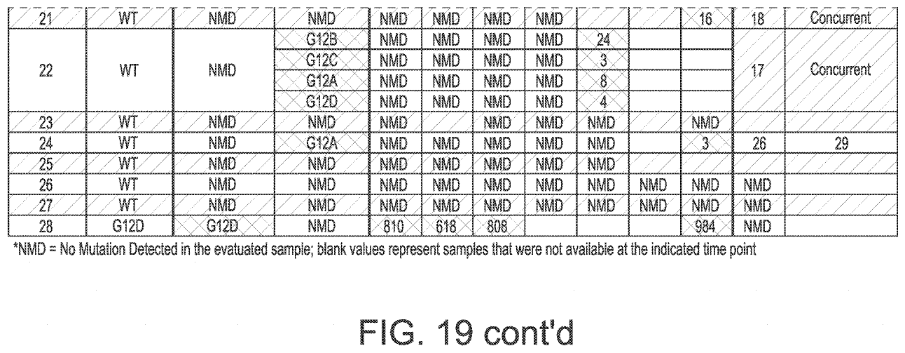

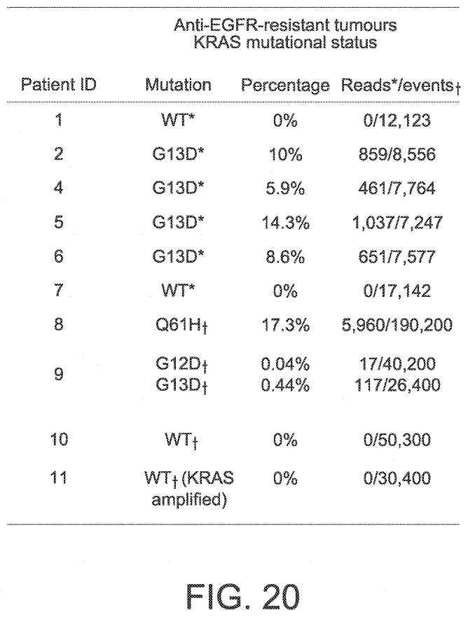

In addition, secondary KRAS mutations have been identified in EGFR blockade resistant patients. RAS is downstream of EGFR and it has been found to constitute a mechanism of resistance to EGFR blockade therapies. EGFR blockade resistant KRAS mutant tumors can be targeted using compositions and methods disclosed herein. In a few cases, more than one KRAS mutation was identified in the same patient (up to four different mutations co-occur). This mutational spectrum appears to be at least somewhat different than primary tumor missense mutants in colorectal cancer. (Diaz et al The molecular evolution of acquired resistance to targeted EGFR blockade in colorectal cancers, Nature 486:537 (2012); Misale et al Emergence of KRAS muations and acquired resistance to anti-EGFR therapy in colorectal cancer, Nature 486:532 (2012)). FIG. 19 depicts secondary KRAS mutations after acquisition of EGFR blockade resistance. (Diaz et at The molecular evolution of acquired resistance to targeted EGFR blockade in colorectal cancers, Nature 486:537 (2012)). FIG. 20 depicts secondary KRAS mutations after EGFR blockade. (Misale et at Emergence of KRAS muations and acquired resistance to anti-EGFR therapy in colorectal cancer, Nature 486:532 (2012)).

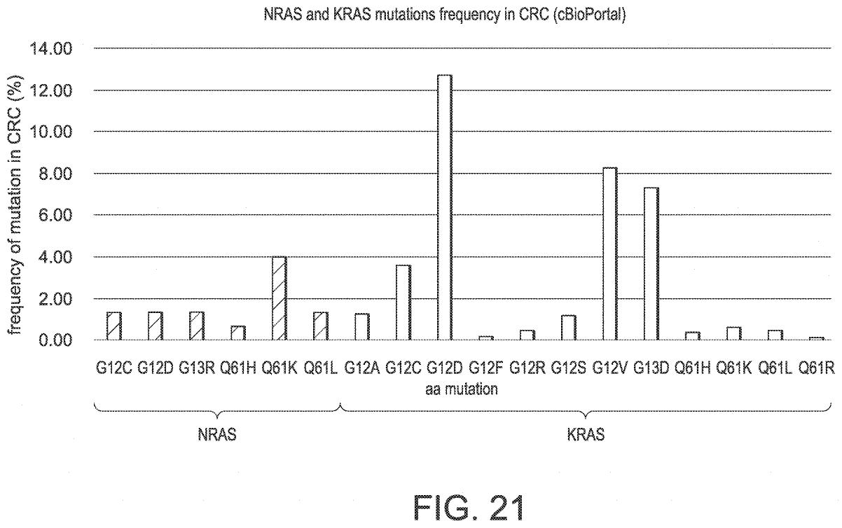

As shown in FIG. 21, NRAS is also mutated in colorectal cancer, but at a lower frequency than KRAS.

In this example, animals are administered an RNA cancer vaccine that includes an mRNA encoding at least one activating oncogene mutation peptide, e.g., at least one activating KRAS mutation. HLA*A*11:01 Tg mice (Taconic, strain 9660F, n=4) or HLA-A*2:01 Tg mice (Taconic, strain 9659F, n=4) are administered mRNA encoding mutated KRAS as follows: mRNA encoding mutated KRAS administered on day 1, bleed taken on day 8, mRNA encoding mutated KRAS administered on day 15, animal sacrificed on day 22. The test groups are shown in Table 6 as follows:

TABLE 6

| | | Test/Control | Genetic | | | Dosing |
|---|---|---|---|---|---|---|
| TEST group | Group | Material | adjuvant | Vehicle | Route | Regimen |
| KRAS-MUT | 1 | KRAS G12D | None (NTFIX) | Compound | IM | Day 1, 15 |
| | | | | 25 | | |
| | 2 | KRAS G12V | None (NTFIX) | Compound | IM | Day 1, 15 |
| | | | | 25 | | |
| | 3 | KRAS G13D | None (NTFIX) | Compound | IM | Day 1, 15 |
| | | | | 25 | | |
| No Ag | 4 | NTFIX | NTFIX | Compound | IM | Day 1, 15 |
| | | | | 25 | | |

mRNA is administered to animals at a dose of 0.5 mg/kg (10ug per 20-g animal). Ex vivo restimulation (1 ug/ml per peptide) is tested for 4 hours at 37 degrees Celsius in the presence of GolgiPlug (Brefeldin A). Intracellular cytokine staining (ICS) is tested for KRAS G12D, KRAS G12V, KRAS G13D, KRAS G12WT, KRAS G13WT, and no peptide.

mRNA encoding KRAS mutations is tested for the ability to generate T cells. Efficacy of mRNA encoding KRAS mutations is compared, for example, to peptide vaccination.

Exemplary KRAS mutant peptide sequences and mRNA constructs are shown in Tables 7-9.

TABLE 7
KRAS mutant peptide sequences

| | 9 AA sequence | 15mer | 25 mer |
|---|---|---|---|
| G12D | VVGADGVGK | KLVVVGADGVGKSAL | MTEYKLVVVGADGVGKSALTIQLIQ |
| | (SEQ ID | (SEQ ID NO: 317) | (SEQ ID NO: 318) |
| | NO: 316) | | |
| G12V | VVGAVGVGK | KLVVVGAVGVGKSAL | MTEYKLVVVGAVGVGKSALTIQLIQ |
| | (SEQ ID | (SEQ ID NO: 320) | (SEQ ID NO: 321) |
| | NO: 319) | | |
| G13D | VGAGDVGKS | LVVVGAGDVGKSALT | MTEYKLVVVGAGDVGKSALTIQLIQ |
| | (SEQ ID | (SEQ ID NO: 323) | (SEQ ID NO: 324) |
| | NO: 322) | | |
| G12C | VVGACGVGK | KLVVVGACGVGKSA | MTEYKLVVVGACGVGKSALTIQLIQ |
| | (SEQ ID | (SEQ ID NO: 326) | (SEQ ID NO: 327) |
| | NO: 325) | | |
| WT | | | MTEYKLVVVGAGGVGKSALTIQLIQ |
| | | | (SEQ ID NO: 328) |

TABLE 8
KRAS mutant amino acid sequences

| KRAS MUTANT | AMINO ACID SEQUENCE |
|---|---|
| KRAS (G12D) | MKLVVVGADGVGKSAL (SEQ ID NO: 329) |
| 15mer | |
| KRAS (G12V) | MKLVVVGAVGVGKSAL (SEQ ID NO: 330) |
| 15mer | |
| KRAS (G13D) | MLVVVGAGDVGKSALT (SEQ ID NO: 331) |
| 15mer | |
| KRAS (G12D) | MTEYKLVVVGADGVGKSALTIQLIQ (SEQ ID NO: 332) |
| 25mer | |
| KRAS (G12V) | MTEYKLVVVGAVGVGKSALTIQLIQ (SEQ ID NO: 333) |
| 25mer | |
| KRAS (G13D) | MTEYKLVVVGAGDVGKSALTIQLIQ (SEQ ID NO: 334) |
| 25mer | |
| KRAS (G12D) | MKLVVVGADGVGKSALKLVVVGADGVGKSALKLVVVGADGVGKSAL |
| 15mer\hat{ }3 | (SEQ ID NO: 335) |
| KRAS (G12V) | MKLVVVGAVGVGKSALKLVVVGAVGVGKSALKLVVVGAVGVGKSAL |
| 15mer\hat{ }3 | (SEQ ID NO: 336) |
| KRAS (G13D) | MLVVVGAGDVGKSALTLVVVGAGDVGKSALTLVVVGAGDVGKSALT |
| 15mer\hat{ }3 | (SEQ ID NO: 337) |
| KRAS (G12D) | MTEYKLVVVGADGVGKSALTIQLIQMTEYKLVVVGADGVGKSALTIQL |
| 25mer\hat{ }3 | IQMTEYKLVVVGADGVGKSALTIQLIQ (SEQ ID NO: 338) |
| KRAS (G12V) | MTEYKLVVVGAVGVGKSALTIQLIQMTEYKLVVVGAVGVGKSALTIQL |
| 25mer\hat{ }3 | IQMTEYKLVVVGAVGVGKSALTIQLIQ (SEQ ID NO: 339) |
| KRAS (G13D) | MTEYKLVVVGAGDVGKSALTIQLIQMTEYKLVVVGAGDVGKSALTIQL |
| 25mer\hat{ }3 | IQMTEYKLVVVGAGDVGKSALTIQLIQ (SEQ ID NO: 340) |
| KRAS (G12C) | MTEYKLVVVGACGVGKSALTIQLIQ (SEQ ID NO: 341) |
| 25mer | |
| KRAS (G12C) | MTEYKLVVVGACGVGKSALTIQLIQMTEYKLVVVGACGVGKSALTIQL |
| 25mer\hat{ }3 | IQMTEYKLVVVGACGVGKSALTIQLIQ (SEQ ID NO: 342) |
| KRAS(WT) 25mer | MTEYKLVVVGAGGVGKSALTIQLIQ (SEQ ID NO: 343) |

TABLE 9
KRAS mutant antigen mRNA sequences

| mRNA | Orf Sequence | |
|---|---|---|
| Name | (Amino Acid) | Orf Sequence (Nucleotide) |
| KRAS | MTEYKLVVVGADGV | ATGACCGAGTACAAGCTGGTGGTGGTGGGCGCCGAC |
| (G12D) | GKSALTIQLIQ (SEQ | GGCGTGGGCAAGAGCGCCCTGACCATCCAGCTGATC |
| 25mer | ID NO: 357) | CAG (SEQ ID NO: 344) |
| KRAS | MTEYKLVVVGAVGV | ATGACCGAGTACAAGCTGGTGGTGGTGGGCGCCGTG |
| (G12V) | GKSALTIQLIQ (SEQ | GGCGTGGGCAAGAGCGCCCTGACCATCCAGCTGATC |
| 25mer | ID NO: 358) | CAG (SEQ ID NO: 345) |
| KRAS | MTEYKLVVVGAGDV | ATGACCGAGTACAAGCTGGTGGTGGTGGGCGCCGGC |
| (G13D) | GKSALTIQLIQ | GACGTGGGCAAGAGCGCCCTGACCATCCAGCTGATC |
| 25mer | (SEQ ID NO: 359) | CAG (SEQ ID NO: 346) |
| KRAS | MTEYKLVVVGADGV | ATGACCGAGTACAAGTTAGTGGTTGTGGGCGCCGAC |
| (G12D) | GKSALTIQLIQMTEY | GGCGTGGGCAAGAGCGCCCTCACCATCCAGCTTATC |
| 25mer\hat{ }3 | KLVVVGADGVGKSA | CAGATGACGGAATATAAGTTAGTAGTAGTGGGAGCC |
| | LTIQLIQMTEYKLVV | GACGGTGTCGGCAAGTCCGCTTTGACCATTCAACTT |
| | VGADGVGKSALTIQL | ATTCAGATGACAGAGTATAAGCTGGTCGTTGTAGGC |
| | IQ (SEQ ID NO: 360) | GCAGACGGCGTTGGAAAGTCGGCACTGACGATCCAG |
| | | TTGATCCAG (SEQ ID NO: 347) |
| KRAS | MTEYKLVVVGAVGV | ATGACCGAGTACAAGCTCGTCGTGGTGGGCGCCGTG |
| (G12V) | GKSALTIQLIQMTEY | GGCGTGGGCAAGAGCGCCCTAACCATCCAGTTGATC |
| 25mer\hat{ }3 | KLVVVGAVGVGKSA | CAGATGACCGAATATAAGCTCGTGGTAGTCGGAGCG |
| | LTIQLIQMTEYKLVV | GTGGGCGTTGGCAAGTCAGCGCTAACAATACAACTA |
| | VGAVGVGKSALTIQL | ATCCAAATGACCGAATACAAGCTAGTTGTAGTCGGT |
| | IQ (SEQ ID NO: 361) | GCCGTCGGCGTTGGAAAGTCAGCCCTTACAATTCAG |
| | | CTCATTCAG (SEQ ID NO: 348) |
| KRAS | MTEYKLVVVGAGDV | ATGACCGAGTACAAGCTCGTAGTGGTTGGCGCCGGC |
| (G13D) | GKSALTIQLIQMTEY | GACGTGGGCAAGAGCGCCCTAACCATCCAGCTCATC |
| 25mer\hat{ }3 | KLVVVGAGDVGKSA | CAGATGACAGAATATAAGCTTGTGGTTGTGGGAGCA |
| | LTIQLIQMTEYKLVV | GGAGACGTGGGAAAGAGTGCGTTGACGATTCAACTC |
| | VGAGDVGKSALTIQL | ATACAGATGACCGAATACAAGTTGGTGGTGGTCGGC |
| | IQ (SEQ ID NO: 362) | GCAGGTGACGTTGGTAAGTCTGCACTAACTATACAA |
| | | CTGATCCAG (SEQ ID NO: 349) |
| KRAS | MTEYKLVVVGACGV | ATGACCGAGTACAAGCTGGTGGTGGTGGGCGCCTGC |
| (G12C) | GKSALTIQLIQ (SEQ | GGCGTGGGCAAGAGCGCCCTGACCATCCAGCTGATC |
| 25mer | ID NO: 363) | CAG (SEQ ID NO: 350) |
| KRAS | MTEYKLVVVGACGV | ATGACCGAGTACAAGCTCGTGGTTGTTGGCGCCTGC |
| (G12C) | GKSALTIQLIQMTEY | GGCGTGGGCAAGAGCGCCCTCACCATCCAGCTCATC |
| 25mer\hat{ }3 | KLVVVGACGVGKSA | CAGATGACAGAGTATAAGTTAGTCGTTGTCGGAGCT |
| | LTIQLIQMTEYKLVV | TGCGGAGTTGGAAAGTCGGCGCTCACCATTCAACTC |
| | VGACGVGKSALTIQL | ATACAAATGACAGAATATAAGTTAGTGGTGGTGGGT |
| | IQ (SEQ ID NO: 364) | GCGTGTGGCGTTGGCAAGAGTGCGCTTACTATCCAG |
| | | CTCATTCAG (SEQ ID NO: 351) |
| KRAS | MTEYKLVVVGAGGV | ATGACCGAGTACAAGCTGGTGGTGGTGGGCGCCGGC |
| (WT) | GKSALTIQLIQ (SEQ | GGCGTGGGCAAGAGCGCCCTGACCATCCAGCTGATC |
| 25mer | ID NO: 365) | CAG (SEQ ID NO: 352) |

Chemistry: uridines modified N1-methyl pseudouridine (m1Ψ)
Cap: C1
Tail: T100
5′ UTR Sequence (standard 5′ Flank (includes Production FP + T7 site + 5′UTR)):
TCAAGCTTTTGGACCCTCGTACAGAAGCTAATACGACTCACTATAGGGAAATAAG
AGAGAAAAGAAGAGTAAGAAGAAATATAAGAGCCACC (SEQ ID NO: 353)
5′ UTR Sequence (No Promoter): GGGAAATAAGAGAGAAAAGAAGAGTAAGAA
GAAATATAAGAGCCACC (SEQ ID NO: 354)
3′ UTR Sequence (Human 3′ UTR no XbaI): TGATAATAGGCTGGAGCCTCGGTGGCCA
TGCTTCTTGCCCCTTGGGCCTCCCCCCAGCCCCTCCTCCCCTTCCTGCACCCGTAC
CCCCGTGGTCTTTGAATAAAGTCTGAGTGGGCGGC (SEQ ID NO: 355)

### Example 8. Recurrent Splice Site and Silent Mutation “Hotspots” in p53

The p53 gene (official symbol TP53) is mutated more frequently than any other gene in human cancers. Large cohort studies have shown that, for most p53 mutations, the genomic position is unique to one or only a few patients and the mutation cannot be used as recurrent neoantigens for therapeutic vaccines designed for a specific population of patients. A small subset of p53 loci do, however, exhibit a “hotspot” pattern, in which several positions in the gene are mutated with relatively high frequency. Strikingly, a large portion of these recurrently mutated regions occur near exon-intron boundaries, disrupting the canonical nucleotide sequence motifs recognized by the mRNA splicing machinery. Mutation of a splicing motif can alter the final mRNA sequence even if no change to the local amino acid sequence is predicted (i.e. for synonymous or intronic mutations). Therefore, these mutations are often annotated as “noncoding” by common annotation tools and neglected for further analysis, even though they may alter mRNA splicing in unpredictable ways and exert severe functional impact on the translated protein. If an alternatively spliced isoform produces an in-frame sequence change (i.e., no PTC is produced), it can escape depletion by NMD and be readily expressed, processed, and presented on the cell surface by the HLA system. Further, mutation-derived alternative splicing is usually “cryptic”, i.e., not expressed in normal tissues, and therefore may be recognized by T-cells as non-self neoantigens.

Several mutation sites were confirmed by RNA-seq to produce retained introns or cryptic splicing. Two representative mutation-derived peptides had multiple HLA-A2 binding epitopes with no matches elsewhere in the coding genome.

Recurrent mutations in p53 that were identified included:

(1) mutations at the canonical 5′ splice site neighboring codon p.T125, inducing a retained intron having peptide sequence TAKSVTCTVSCPEGLASMRLQCLAVSPCISFVWNFGIPLHPLASCQCFFIVYPLNV (SEQ ID NO: 232) that contains epitopes AVSPCISFVW (SEQ ID NO: 233) (HLA-B*57:01, HLA-B*58:01), HPLASCQCFF (SEQ ID NO: 234) (HLA-B*35:01, HLA-B*53:01), FVWNFGIPL (SEQ ID NO: 235) (HLA-A*02:01, HLA-A*02:06, HLA-B*35:01);
(2) mutations at the canonical 5′ splice site neighboring codon p.331, inducing a retained intron having peptide sequence EYFTLQVLSLGTSYQVESFQSNTQNAVFFLTVLPAIGAFAIRGQ (SEQ ID NO: 236) that contains epitopes LQVLSLGTSY (SEQ ID NO: 237) (HLA-B*15:01), FQSNTQNAVF (SEQ ID NO: 238) (HLA-B*15:01);
(3) mutations at the canonical 3′ splice site neighboring codon p.126, inducing a cryptic alternative exonic 3′ splice site producing the novel spanning peptide sequence AKSVTCTMFCQLAK (SEQ ID NO: 239) that contains epitopes CTMFCQLAK (SEQ ID NO: 240) (HLA-A*11:01), KSVTCTMF (SEQ ID NO: 241) (HLA-B*58:01); and (4) mutations at the canonical 5′ splice site neighboring codon p.224, inducing a cryptic alternative intronic 5′ splice site producing the novel spanning peptide sequence VPYEPPEVWLALTVPPSTAWAA (SEQ ID NO: 242) that contains epitopes VPYEPPEVW (SEQ ID NO: 243 (HLA-B*53:01, HLA-B*51:01), LTVPPSTAW (SEQ ID NO: 244) (HLA-B*58:01, HLA-B*57:01), wherein the transcript codon positions refer to the canonical full-length p53 transcript ENST00000269305 (SEQ ID NO: 245) from the Ensembl v83 human genome annotation.

### EQUIVALENTS

Those skilled in the art will recognize, or be able to ascertain using no more than routine experimentation, many equivalents to the specific embodiments of the disclosure described herein. Such equivalents are intended to be encompassed by the following claims.

The term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

All references, including patent documents, disclosed herein are incorporated by reference in their entirety.

What is claimed is:

1. An mRNA cancer vaccine, comprising:
a lipid nanoparticle comprising one or more of the following:
(a) one or more mRNA each having one or more open reading frames encoding 1-500 peptide epitopes which are personalized cancer antigens and a universal type II T-cell epitope;
(b) one or more mRNA each having an open reading frame encoding an activating oncogene mutation peptide, optionally wherein the mRNA further comprises a universal type II T-cell epitope;
(c) one or more mRNA each having an open reading frame encoding a cancer antigen peptide epitope, wherein the mRNA vaccine encodes 5-100 peptide epitopes and at least two of the peptide epitopes are personalized cancer antigens, optionally wherein the mRNA further comprises a universal type II T-cell epitope; and/or
(d) one or more mRNA each having an open reading frame encoding a cancer antigen peptide epitope, wherein the mRNA vaccine encodes 5-100 peptide epitopes and at least three of the peptide epitopes are complex variants and at least two of the peptide epitopes are point mutations, optionally wherein the mRNA further comprises a universal type II T-cell epitope.

2. The mRNA cancer vaccine of claim 1, wherein the mRNA cancer vaccine encodes 1-20 universal type II T-cell epitopes.

3. The mRNA cancer vaccine of claim 2, wherein the universal type II T-cell epitope is selected from the group consisting of: ILMQYIKANSKFIGI (Tetanus toxin; SEQ ID NO: 226), FNNFTVSFWLRVPKVSASHLE, (Tetanus toxin; SEQ ID NO: 227), QYIKANSKFIGITE (Tetanus toxin; SEQ ID NO: 228) QSIALSSLMVAQAIP (Diptheria toxin; SEQ ID NO: 229), and AKFVAAWTLKAAA (pan-DR epitope; SEQ ID NO: 230).

4. The mRNA cancer vaccine of any one of claims 1-3, wherein the universal type II T-cell epitope is the same universal type II T-cell epitope throughout the mRNA.

5. The mRNA cancer vaccine of any one of claims 1-3, wherein the universal type II T-cell epitope is repeated 1-20 times in the mRNA.

6. The mRNA cancer vaccine of any one of claims 1-3, wherein the universal type II T-cell epitopes are different from one another throughout the mRNA.

7. The mRNA cancer vaccine of any one of claims 1-3, wherein the universal type II T-cell epitope is located between every cancer antigen peptide epitope.

8. The mRNA cancer vaccine of any one of claims 1-3, wherein the universal type II T-cell epitope is located between every other cancer antigen peptide epitope.

9. The mRNA cancer vaccine of any one of claims 1-3, wherein the universal type II T-cell epitope is located between every third cancer antigen peptide epitope.

10. The mRNA cancer vaccine of any preceding claim, wherein one or more of the following conditions are met:
(i) the activating oncogene mutation is a KRAS mutation;
(ii) the KRAS mutation is a G12 mutation, optionally wherein the G12 KRAS mutation is selected from a G12D, G12V, G12S, G12C, G12A, and a G12R KRAS mutation;
(iii) the KRAS mutation is a G13 mutation, optionally wherein the G13 KRAS mutation is a G13D KRAS mutation; and/or
(iv) the activating oncogene mutation is a H-RAS or N-RAS mutation.

11. The mRNA cancer vaccine of any preceding claim, wherein one or more of the following conditions are met:
(A) the mRNA has an open reading frame encoding a concatemer of two or more activating oncogene mutation peptides;
(B) at least two of the peptide epitopes are separated from one another by a single Glycine, optionally wherein all of the peptide epitopes are separated from one another by a single Glycine;
(C) the concatemer comprises 3-10 activating oncogene mutation peptides; and/or
(D) at least two of the peptide epitopes are linked directly to one another without a linker.

12. The mRNA cancer vaccine of any preceding claim, wherein one or more of the following conditions are met:
(i) at least one of the peptide epitopes is a traditional cancer antigen;
(ii) at least one of the peptide epitopes is a recurrent polymorphism;
(iii) the recurrent polymorphism comprises a recurrent somatic cancer mutation in p53;
(iv) the recurrent somatic cancer mutation in p53 is selected from the group consisting of:
(A) mutations at the canonical 5′ splice site neighboring codon p.T125, inducing a retained intron having peptide sequence TAKSVTCTVSCPEGLASMRLQCLAVSPCISFVWNFGIPLHPLASCQCFFIVYPL NV (SEQ ID NO: 232) that contains epitopes AVSPCISFVW (SEQ ID NO: 233) (HLA-B*57:01, HLA-B*58:01), HPLASCQCFF (SEQ ID NO: 234) (HLA-B*35:01, HLA-B*53:01), FVWNFGIPL (SEQ ID NO: 235) (HLA-A*02:01, HLA-A*02:06, HLA-B*35:01);
(B) mutations at the canonical 5′ splice site neighboring codon p.331, inducing a retained intron having peptide sequence EYFTLQVLSLGTSYQVESFQSNTQNAVFFLTVLPAIGAFAIRGQ (SEQ ID NO: 236) that contains epitopes LQVLSLGTSY (SEQ ID NO: 237) (HLA-B*15:01), FQSNTQNAVF (SEQ ID NO: 238) (HLA-B*15:01);
(C) mutations at the canonical 3′ splice site neighboring codon p.126, inducing a cryptic alternative exonic 3′ splice site producing the novel spanning peptide sequence AKSVTCTMFCQLAK (SEQ ID NO: 239) that contains epitopes CTMFCQLAK (SEQ ID NO: 240) (HLA-A*11:01), KSVTCTMF (SEQ ID NO: 241) (HLA-B*58:01); and/or
(D) mutations at the canonical 5′ splice site neighboring codon p.224, inducing a cryptic alternative intronic 5′ splice site producing the novel spanning peptide sequence VPYEPPEVWLALTVPPSTAWAA (SEQ ID NO: 242) that contains epitopes VPYEPPEVW (SEQ ID NO: 243) (HLA-B*53:01, HLA-B*51:01), LTVPPSTAW (SEQ ID NO: 244) (HLA-B*58:01, HLA-B*57:01),
wherein the transcript codon positions refer to the canonical full-length p53 transcript ENST00000269305 (SEQ ID NO: 245) from the Ensembl v83 human genome annotation; and/or
(v) the mRNA cancer vaccine does not comprise a stabilizing agent.

13. The mRNA cancer vaccine of any one of claims 1-3, wherein the one or more mRNA further comprise an open reading frame encoding an immune potentiator.

14. The mRNA cancer vaccine of claim 13, wherein the immune potentiator is formulated in the lipid nanoparticle.

15. The mRNA cancer vaccine of claim 13, wherein the immune potentiator is formulated in a separate lipid nanoparticle.

16. The mRNA cancer vaccine of claim 13, wherein the immune potentiator is a constitutively active human STING polypeptide.

17. The mRNA cancer vaccine of claim 13, wherein the constitutively active human STING polypeptide comprises the amino acid sequence shown in SEQ ID NO: 1.

18. The mRNA cancer vaccine of claim 13, wherein the mRNA encoding the constitutively active human STING polypeptide comprises the nucleotide sequence shown in SEQ ID NO: 170.

19. The mRNA cancer vaccine of claim 13, wherein the mRNA encoding the constitutively active human STING polypeptide comprises a 3′ UTR having a miR-122 microRNA binding site.

20. The mRNA cancer vaccine of claim 19, wherein the miR-122 microRNA binding site comprises the nucleotide sequence shown in SEQ ID NO: 175.

21. The mRNA cancer vaccine of any one claims 1-20, wherein the one or more mRNA each comprise a 5′ UTR comprising the nucleotide sequence set forth in SEQ ID NO: 176.

22. The mRNA cancer vaccine of any one of claims 1-21, wherein the one or more mRNA each comprise a poly A tail.

23. The mRNA cancer vaccine of claim 22, wherein the poly A tail comprises about 100 nucleotides.

24. The mRNA cancer vaccine of any one of claims 1-23, wherein the one or more mRNA each comprise a 5′ Cap 1 structure.

25. The mRNA cancer vaccine of any one of claims 1-24, wherein the one or more mRNA comprise at least one chemical modification.

26. The mRNA cancer vaccine of claim 25, wherein the chemical modification is N1-methylpseudouridine.

27. The mRNA cancer vaccine of claim 26, wherein the one or more mRNA is fully modified with N1-methylpseudouridine.

28. The mRNA cancer vaccine of any one of claims 1-27, wherein the one or more mRNA encode 45-55 personalized cancer antigens.

29. The mRNA cancer vaccine of any one of claims 1-27, wherein the one or more mRNA encode 52 personalized cancer antigens.

30. The mRNA cancer vaccine of any one of claims 1-27, wherein each of the personalized cancer antigens is encoded by a separate open reading frame.

31. The mRNA cancer vaccine of any one of claims 1-27, wherein the peptide epitopes are in the form of a concatemeric cancer antigen comprised of 2-100 peptide epitopes, optionally wherein the concatemeric cancer antigen is comprised of 5-100 peptide epitopes.

32. The mRNA cancer vaccine of claim 31, wherein the concatemeric cancer antigen comprises one or more of:
a) the 2-100 peptide epitopes or, optionally, 5-100 peptide epitopes are interspersed by cleavage sensitive sites;
b) the mRNA encoding each peptide epitope is linked directly to one another without a linker;
c) the mRNA encoding each peptide epitope is linked to one or another with a single nucleotide linker;
d) each peptide epitope comprises 25-35 amino acids and includes a centrally located SNP mutation;
e) at least 30% of the peptide epitopes have a highest affinity for class I MHC molecules from a subject;
f) at least 30% of the peptide epitopes have a highest affinity for class II MHC molecules from a subject;
g) at least 50% of the peptide epitopes have a predicated binding affinity of IC>500 nM for HLA-A, HLA-B and/or DRB1;
h) the mRNA encodes 45-55 peptide epitopes;
i) the mRNA encodes 52 peptide epitopes;
j) 50% of the peptide epitopes have a binding affinity for class I MHC and 50% of the peptide epitopes have a binding affinity for class II MHC;
k) the mRNA encoding the peptide epitopes is arranged such that the peptide epitopes are ordered to minimize pseudo-epitopes,
l) at least 30% of the peptide epitopes are class I MHC binding peptides of 15 amino acids in length; and/or
m) at least 30% of the peptide epitopes are class II MHC binding peptides of 21 amino acids in length.

33. An mRNA cancer vaccine, comprising:
one or more mRNA each having one or more open reading frames encoding 45-55 peptide epitopes which are personalized cancer antigens formulated in a lipid nanoparticle; optionally wherein at least one of the peptide epitopes is an activating oncogene mutation peptide or a traditional cancer antigen, and optionally wherein at least three of the peptide epitopes are complex variants and at least two of the peptide epitopes are point mutations.

34. The mRNA cancer vaccine of 33, wherein the one or more mRNA encode 48-54 personalized cancer antigens.

35. The mRNA cancer vaccine of any one of claims 33-34, wherein the one or more mRNA encode 52 personalized cancer antigens.

36. The mRNA cancer vaccine of any one of claims 33-35, wherein each of the personalized cancer antigens is encoded by a separate open reading frame.

37. The mRNA cancer vaccine of any one of claims 33-35, wherein the peptide epitopes are in the form of a concatemeric cancer antigen comprised of 2-100 peptide epitopes, optionally wherein the concatemeric cancer antigen is comprised of 5-100 peptide epitopes.

38. The mRNA cancer vaccine of claim 37, wherein the concatemeric cancer antigen comprises one or more of:
a) the 2-100 peptide epitopes or, optionally, 5-100 peptide epitopes are interspersed by cleavage sensitive sites;
b) the mRNA encoding each peptide epitope is linked directly to one another without a linker;
c) the mRNA encoding each peptide epitope is linked to one or another with a single nucleotide linker;
d) each peptide epitope comprises 25-35 amino acids and includes a centrally located SNP mutation;
e) at least 30% of the peptide epitopes have a highest affinity for class I MHC molecules from a subject;
f) at least 30% of the peptide epitopes have a highest affinity for class II MHC molecules from a subject;
g) at least 50% of the peptide epitopes have a predicated binding affinity of IC>500 nM for HLA-A, HLA-B and/or DRB1;
h) the mRNA encodes 45-55 peptide epitopes;
i) the mRNA encodes 52 peptide epitopes;
j) 50% of the peptide epitopes have a binding affinity for class I MHC and 50% of the peptide epitopes have a binding affinity for class II MHC;
k) the mRNA encoding the peptide epitopes is arranged such that the peptide epitopes are ordered to minimize pseudo-epitopes,
l) at least 30% of the peptide epitopes are class I MHC binding peptides of 15 amino acids in length; and/or
m) at least 30% of the peptide epitopes are class II MHC binding peptides of 21 amino acids in length.

39. The mRNA cancer vaccine any one of claims 33-38, wherein at least two of the peptide epitopes are separated from one another by a universal type II T-cell epitope.

40. The mRNA cancer vaccine any one of claims 33-38, wherein all of the peptide epitopes are separated from one another by a universal type II T-cell epitope.

41. The mRNA cancer vaccine any one of claims 33-38, wherein the mRNA cancer vaccine encodes 1-20 universal type II T-cell epitopes.

42. The mRNA cancer vaccine of claim 41, wherein the universal type II T-cell epitope is selected from the group consisting of: ILMQYIKANSKFIGI (Tetanus toxin; SEQ ID NO: 226), FNNFTVSFWLRVPKVSASHLE, (Tetanus toxin; SEQ ID NO: 227), QYIKANSKFIGITE (Tetanus toxin; SEQ ID NO: 228) QSIALSSLMVAQAIP (Diptheria toxin; SEQ ID NO: 229), and AKFVAAWTLKAAA (pan-DR epitope; SEQ ID NO: 230).

43. The mRNA cancer vaccine of any one of claims 39-42, wherein the universal type II T-cell epitope is the same universal type II T-cell epitope throughout the mRNA.

44. The mRNA cancer vaccine of any one of claims 39-42, wherein the universal type II T-cell epitope is repeated 1-20 times in the mRNA.

45. The mRNA cancer vaccine of any one of claims 39-42, wherein the universal type II T-cell epitopes are different from one another throughout the mRNA.

46. The mRNA cancer vaccine of any one of claims 39-42, wherein the universal type II T-cell epitope is located between every peptide epitope.

47. The mRNA cancer vaccine of any one of claims 39-42, wherein the universal type II T-cell epitope is located between every other peptide epitope.

48. The mRNA cancer vaccine of any one of claims 39-42, wherein the universal type II T-cell epitope is located between every third peptide epitope.

49. The mRNA cancer vaccine of any one of claims 33-48, wherein the one or more mRNA further comprise an open reading frame encoding an immune potentiator.

50. The mRNA cancer vaccine of claim 38, wherein the immune potentiator is formulated in the lipid nanoparticle.

51. The mRNA cancer vaccine of claim 38, wherein the immune potentiator is formulated in a separate lipid nanoparticle.

52. The mRNA cancer vaccine of claim 38, wherein the immune potentiator is a constitutively active human STING polypeptide.

53. The mRNA cancer vaccine of claim 52, wherein the constitutively active human STING polypeptide comprises the amino acid sequence shown in SEQ ID NO: 1.

54. The mRNA cancer vaccine of claim 52, wherein the mRNA encoding the constitutively active human STING polypeptide comprises the nucleotide sequence shown in SEQ ID NO: 170.

55. The mRNA cancer vaccine of any one of claims 33-54, wherein one or more of the following conditions are met:
(i) the activating oncogene mutation is a KRAS mutation;
(ii) the KRAS mutation is a G12 mutation, optionally wherein the G12 KRAS mutation is selected from a G12D, G12V, G12S, G12C, G12A, and a G12R KRAS mutation;
(iii) the KRAS mutation is a G13 mutation, optionally wherein the G13 KRAS mutation is a G13D KRAS mutation; and/or
(iv) the activating oncogene mutation is a H-RAS or N-RAS mutation.

56. The mRNA cancer vaccine of any one of claims 33-55, wherein one or more of the following conditions are met:
(A) the mRNA has an open reading frame encoding a concatemer of two or more activating oncogene mutation peptides;
(B) at least two of the peptide epitopes are separated from one another by a single Glycine, optionally wherein all of the peptide epitopes are separated from one another by a single Glycine;
(C) the concatemer comprises 3-10 activating oncogene mutation peptides; and/or
(D) at least two of the peptide epitopes are linked directly to one another without a linker.

57. The mRNA cancer vaccine of any one of claims 33-56, wherein one or more of the following conditions are met:
(i) at least one of the peptide epitopes is a traditional cancer antigen;
(ii) at least one of the peptide epitopes is a recurrent polymorphism;
(iii) the recurrent polymorphism comprises a recurrent somatic cancer mutation in p53;
(iv) the recurrent somatic cancer mutation in p53 is selected from the group consisting of:
(A) mutations at the canonical 5′ splice site neighboring codon p.T125, inducing a retained intron having peptide sequence TAKSVTCTVSCPEGLASMRLQCLAVSPCISFVWNFGIPLHPLASCQCFFIVYPL NV (SEQ ID NO: 232) that contains epitopes AVSPCISFVW (SEQ ID NO: 233) (HLA-B*57:01, HLA-B*58:01), HPLASCQCFF (SEQ ID NO: 234) (HLA-B*35:01, HLA-B*53:01), FVWNFGIPL (SEQ ID NO: 235) (HLA-A*02:01, HLA-A*02:06, HLA-B*35:01);
(B) mutations at the canonical 5′ splice site neighboring codon p.331, inducing a retained intron having peptide sequence EYFTLQVLSLGTSYQVESFQSNTQNAVFFLTVLPAIGAFAIRGQ (SEQ ID NO: 236) that contains epitopes LQVLSLGTSY (SEQ ID NO: 237) (HLA-B*15:01), FQSNTQNAVF (SEQ ID NO: 238) (HLA-B*15:01);
(C) mutations at the canonical 3′ splice site neighboring codon p.126, inducing a cryptic alternative exonic 3′ splice site producing the novel spanning peptide sequence AKSVTCTMFCQLAK (SEQ ID NO: 239) that contains epitopes CTMFCQLAK (SEQ ID NO: 240) (HLA-A*11:01), KSVTCTMF (SEQ ID NO: 241) (HLA-B*58:01); and/or
(D) mutations at the canonical 5′ splice site neighboring codon p.224, inducing a cryptic alternative intronic 5′ splice site producing the novel spanning peptide sequence VPYEPPEVWLALTVPPSTAWAA (SEQ ID NO: 242) that contains epitopes VPYEPPEVW (SEQ ID NO: 243) (HLA-B*53:01, HLA-B*51:01), LTVPPSTAW (SEQ ID NO: 244) (HLA-B*58:01, HLA-B*57:01),
wherein the transcript codon positions refer to the canonical full-length p53 transcript ENST00000269305 (SEQ ID NO: 245) from the Ensembl v83 human genome annotation; and/or
(v) the mRNA cancer vaccine does not comprise a stabilizing agent.

58. An mRNA cancer vaccine, comprising:
a lipid nanoparticle comprising:
(i) one or more mRNA each having one or more open reading frames encoding 1-500 peptide epitopes which are personalized cancer antigens, and
(ii) an mRNA having an open reading frame encoding a polypeptide that enhances an immune response to the personalized cancer antigens,
optionally wherein (i) and (ii) are present at mass ratio of approximately 5:1; optionally wherein at least one of the peptide epitopes is an activating oncogene mutation peptide or a traditional cancer antigen, and optionally wherein at least three of the peptide epitopes are complex variants and at least two of the peptide epitopes are point mutations.

59. The mRNA cancer vaccine of claim 58, wherein the immune response comprises a cellular or humoral immune response characterized by:
(i) stimulating Type I interferon pathway signaling;
(ii) stimulating NFkB pathway signaling;
(iii) stimulating an inflammatory response;
(iv) stimulating cytokine production; or
(v) stimulating dendritic cell development, activity or mobilization; and
(vi) a combination of any of (i)-(vi).

60. The mRNA cancer vaccine of claim 58, which comprises a single mRNA construct encoding both the peptide epitopes and the polypeptide that enhances an immune response to the personalized cancer antigens.

61. The mRNA cancer vaccine of claim 58 or 59, wherein the peptide epitopes are in the form of a concatemeric cancer antigen comprised of 2-100 peptide epitopes, optionally wherein the concatemeric cancer antigen is comprised of 5-100 peptide epitopes.

62. The mRNA cancer vaccine of claim 61, wherein the concatemeric cancer antigen comprises one or more of:
a) the 2-100 peptide epitopes or, optionally, 5-100 peptide epitopes are interspersed by cleavage sensitive sites;
b) the mRNA encoding each peptide epitope is linked directly to one another without a linker;
c) the mRNA encoding each peptide epitope is linked to one or another with a single nucleotide linker;
d) each peptide epitope comprises 25-35 amino acids and includes a centrally located SNP mutation;
e) at least 30% of the peptide epitopes have a highest affinity for class I MHC molecules from a subject;
f) at least 30% of the peptide epitopes have a highest affinity for class II MHC molecules from a subject;
g) at least 50% of the peptide epitopes have a predicated binding affinity of IC>500 nM for HLA-A, HLA-B and/or DRB1;
h) the mRNA encodes 45-55 peptide epitopes;
i) the mRNA encodes 52 peptide epitopes;
j) 50% of the peptide epitopes have a binding affinity for class I MHC and 50% of the peptide epitopes have a binding affinity for class II MHC;
k) the mRNA encoding the peptide epitopes is arranged such that the peptide epitopes are ordered to minimize pseudo-epitopes,
l) at least 30% of the peptide epitopes are class I MHC binding peptides of 15 amino acids in length; and/or
m) at least 30% of the peptide epitopes are class II MHC binding peptides of 21 amino acids in length.

63. The mRNA cancer vaccine of claim 62, wherein each peptide epitope comprises a centrally located SNP mutation with 7-15 flanking amino acids on each side of the SNP mutation.

64. The mRNA cancer vaccine of any one of claims 58-63, wherein the polypeptide that enhances an immune response to at least one personalized cancer antigens in a subject is a constitutively active human STING polypeptide.

65. The mRNA cancer vaccine of claim 64, wherein the constitutively active human STING polypeptide comprises one or more mutations selected from the group consisting of V147L, N154S, V155M, R284M, R284K, R284T, E315Q, R375A, and combinations thereof.

66. The mRNA cancer vaccine of claim 65, wherein the constitutively active human STING polypeptide comprises a V155M mutation.

67. The mRNA cancer vaccine of claim 65, wherein the constitutively active human STING polypeptide comprises mutations R284M/V147L/N154S/V155M.

68. The mRNA cancer vaccine of any one of claims 58-67, wherein each mRNA is formulated in the same or different lipid nanoparticle.

69. The mRNA cancer vaccine of claim 68, wherein each mRNA encoding a cancer personalized cancer antigens is formulated in the same or different lipid nanoparticle.

70. The mRNA cancer vaccine of claim 69, wherein each mRNA encoding a polypeptide that enhances an immune response to the personalized cancer antigens is formulated in the same or different lipid nanoparticle.

71. The mRNA cancer vaccine of any one of claims 68-70, wherein each mRNA encoding a personalized cancer antigen is formulated in the same lipid nanoparticle, and each mRNA encoding a polypeptide that enhances an immune response to the personalized cancer antigen is formulated in a different lipid nanoparticle.

72. The mRNA cancer vaccine of any one of claims 68-70, wherein each mRNA encoding a personalized cancer antigen is formulated in the same lipid nanoparticle, and each mRNA encoding a polypeptide that enhances an immune response to the personalized cancer antigen is formulated in the same lipid nanoparticle as each mRNA encoding a personalized cancer antigen.

73. The mRNA cancer vaccine one of claims 68-70, wherein each mRNA encoding a personalized cancer antigen is formulated in a different lipid nanoparticle, and each mRNA encoding a polypeptide that enhances an immune response to the personalized cancer antigen is formulated in the same lipid nanoparticle as each mRNA encoding each personalized cancer antigen.

74. The mRNA cancer vaccine of any one of claims 1-73, wherein the peptide epitopes are T cell epitopes and/or B cell epitopes.

75. The mRNA cancer vaccine of any one of claims 1-73, wherein the peptide epitopes comprise a combination of T cell epitopes and B cell epitopes.

76. The mRNA cancer vaccine of any one of claims 1-73, wherein at least 1 of the peptide epitopes is a T cell epitope.

77. The mRNA cancer vaccine of any one of claims 1-73, wherein at least 1 of the peptide epitopes is a B cell epitope.

78. The mRNA cancer vaccine of any one of claims 1-73, wherein the peptide epitopes have been optimized for binding strength to a MHC of the subject.

79. The mRNA cancer vaccine of claim 78, wherein a TCR face for each epitope has a low similarity to endogenous proteins.

80. The mRNA cancer vaccine of any one of claims 1-73, further comprising a recall antigen.

81. The mRNA cancer vaccine of claim 80, wherein the recall antigen is an infectious disease antigen.

82. The mRNA cancer vaccine of any one of claims 1-73, further comprising an mRNA having an open reading frame encoding one or more traditional cancer antigens.

83. The mRNA cancer vaccine of any one of claims 58-82, wherein one or more of the following conditions are met:
(i) the activating oncogene mutation is a KRAS mutation;
(ii) the KRAS mutation is a G12 mutation, optionally wherein the G12 KRAS mutation is selected from a G12D, G12V, G12S, G12C, G12A, and a G12R KRAS mutation;
(iii) the KRAS mutation is a G13 mutation, optionally wherein the G13 KRAS mutation is a G13D KRAS mutation; and/or
(iv) the activating oncogene mutation is a H-RAS or N-RAS mutation.

84. The mRNA cancer vaccine of any one of claims 58-83, wherein one or more of the following conditions are met:
(A) the mRNA has an open reading frame encoding a concatemer of two or more activating oncogene mutation peptides;
(B) at least two of the peptide epitopes are separated from one another by a single Glycine, optionally wherein all of the peptide epitopes are separated from one another by a single Glycine;
(C) the concatemer comprises 3-10 activating oncogene mutation peptides; and/or
(D) at least two of the peptide epitopes are linked directly to one another without a linker.

85. The mRNA cancer vaccine of any one of claims 58-84, wherein one or more of the following conditions are met:
(i) at least one of the peptide epitopes is a traditional cancer antigen;
(ii) at least one of the peptide epitopes is a recurrent polymorphism;
(iii) the recurrent polymorphism comprises a recurrent somatic cancer mutation in p53;
(iv) the recurrent somatic cancer mutation in p53 is selected from the group consisting of:
(A) mutations at the canonical 5′ splice site neighboring codon p.T125, inducing a retained intron having peptide sequence TAKSVTCTVSCPEGLASMRLQCLAVSPCISFVWNFGIPLHPLASCQCFFIVYPL NV (SEQ ID NO: 232) that contains epitopes AVSPCISFVW (SEQ ID NO: 233) (HLA-B*57:01, HLA-B*58:01), HPLASCQCFF (SEQ ID NO: 234) (HLA-B*35:01, HLA-B*53:01), FVWNFGIPL (SEQ ID NO: 235) (HLA-A*02:01, HLA-A*02:06, HLA-B*35:01);
(B) mutations at the canonical 5′ splice site neighboring codon p.331, inducing a retained intron having peptide sequence EYFTLQVLSLGTSYQVESFQSNTQNAVFFLTVLPAIGAFAIRGQ (SEQ ID NO: 236) that contains epitopes LQVLSLGTSY (SEQ ID NO: 237) (HLA-B*15:01), FQSNTQNAVF (SEQ ID NO: 238) (HLA-B*15:01);
(C) mutations at the canonical 3′ splice site neighboring codon p.126, inducing a cryptic alternative exonic 3′ splice site producing the novel spanning peptide sequence AKSVTCTMFCQLAK (SEQ ID NO: 239) that contains epitopes CTMFCQLAK (SEQ ID NO: 240) (HLA-A*11:01), KSVTCTMF (SEQ ID NO: 241) (HLA-B*58:01); and/or
(D) mutations at the canonical 5′ splice site neighboring codon p.224, inducing a cryptic alternative intronic 5′ splice site producing the novel spanning peptide sequence VPYEPPEVWLALTVPPSTAWAA (SEQ ID NO: 242) that contains epitopes VPYEPPEVW (SEQ ID NO: 243) (HLA-B*53:01, HLA-B*51:01), LTVPPSTAW (SEQ ID NO: 244) (HLA-B*58:01, HLA-B*57:01),
wherein the transcript codon positions refer to the canonical full-length p53 transcript ENST00000269305 (SEQ ID NO: 245) from the Ensembl v83 human genome annotation; and/or
(v) the mRNA cancer vaccine does not comprise a stabilizing agent.

86. The mRNA cancer vaccine of any one of claims 1-85, wherein the lipid nanoparticle comprises a molar ratio of about 20-60% ionizable amino lipid:5-25% neutral lipid:25-55% sterol; and 0.5-15% PEG-modified lipid, optionally wherein the ionizable amino lipid is a cationic lipid.

87. The mRNA cancer vaccine of claim 86, wherein the lipid nanoparticle comprises a molar ratio of about 50% compound 25:about 10% DSPC:about 38.5% cholesterol; and about 1.5% PEG-DMG.

88. The mRNA cancer vaccine of claim 86, wherein the ionizable amino lipid is selected from the group consisting of for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

89. The mRNA cancer vaccine of any one of claims 1-85, wherein the lipid nanoparticle comprises a compound of Formula (I).

90. The mRNA cancer vaccine of claim 89, wherein the compound of Formula (I) is Compound 25.

91. The mRNA cancer vaccine of any one of claims 1-85, wherein the lipid nanoparticle has a polydispersity value of less than 0.4.

92. The mRNA cancer vaccine of any one of claims 1-85, wherein the lipid nanoparticle has a net neutral charge at a neutral pH value.

93. The mRNA cancer vaccine of any one of claims 1-92, wherein a TCR face for each epitope has a low similarity to endogenous proteins.

94. The mRNA cancer vaccine of any one of claims 1-93, wherein the mRNA further comprises an open reading frame encoding an immune checkpoint modulator.

95. The mRNA cancer vaccine of any one of claims 1-93, further comprising an additional cancer therapeutic agent; optionally wherein the additional cancer therapeutic agent is an immune checkpoint modulator.

96. The mRNA cancer vaccine of claim 93 or 94, wherein the immune checkpoint modulator is an inhibitory checkpoint polypeptide.

97. The mRNA cancer vaccine of claim 96, wherein the inhibitory checkpoint polypeptide inhibits PD1, PD-L1, CTLA4, TIM-3, VISTA, A2AR, B7-H3, B7-H4, BTLA, IDO, KIR, LAG3, or a combination thereof.

98. The mRNA cancer vaccine of claim 97, wherein the checkpoint inhibitor polypeptide is an antibody.

99. The mRNA cancer vaccine of claim 98, wherein the inhibitory checkpoint polypeptide is an antibody selected from an anti-CTLA4 antibody or antigen-binding fragment thereof that specifically binds CTLA4, an anti-PD 1 antibody or antigen-binding fragment thereof that specifically binds PD1, an anti-PD-L1 antibody or antigen-binding fragment thereof that specifically binds PD-L1, and a combination thereof.

100. The mRNA cancer vaccine of claim 99, wherein the checkpoint inhibitor polypeptide is an anti-PD-L1 antibody selected from atezolizumab, avelumab, or durvalumab.

101. The mRNA cancer vaccine of claim 99, wherein the checkpoint inhibitor polypeptide is an anti-CTLA-4 antibody selected from tremelimumab or ipilimumab.

102. The mRNA cancer vaccine of claim 99, wherein the checkpoint inhibitor polypeptide is an anti-PD1 antibody selected from nivolumab or pembrolizumab.

103. The mRNA cancer vaccine of any one of claims 25-102, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

104. A method for vaccinating a subject, comprising:
administering to a subject having cancer the mRNA cancer vaccine of any one of claims 1-103.

105. The method of claim 104, wherein the mRNA vaccine is administered at a dosage level sufficient to deliver between 10 μg and 400 μg of the mRNA vaccine to the subject.

106. The method of claim 105, wherein the mRNA vaccine is administered at a dosage level sufficient to deliver 0.033 mg, 0.1 mg, 0.2 mg, or 0.4 mg to the subject.

107. The method of claim 104 or 105, wherein the mRNA vaccine is administered to the subject twice, three times, four times or more.

108. The method of claim 107, wherein the mRNA vaccine is administered once a day every three weeks.

109. The method of any one of claims 104-108, wherein the mRNA vaccine is administered by intradermal, intramuscular, and/or subcutaneous administration.

110. The method of claim 109, wherein the mRNA vaccine is administered by intramuscular administration.

111. The method of any one of claims 104-110, further comprising administering an additional cancer therapeutic agent; optionally wherein the additional cancer therapeutic agent is an immune checkpoint modulator to the subject.

112. The method of claim 111, wherein the immune checkpoint modulator is an inhibitory checkpoint polypeptide.

113. The method of claim 112, wherein the inhibitory checkpoint polypeptide inhibits PD1, PD-L1, CTLA4, TIM-3, VISTA, A2AR, B7-H3, B7-H4, BTLA, IDO, KIR, LAG3, or a combination thereof.

114. The method of claim 112, wherein the checkpoint inhibitor polypeptide is an antibody.

115. The method of claim 114, wherein the inhibitory checkpoint polypeptide is an antibody selected from an anti-CTLA4 antibody or antigen-binding fragment thereof that specifically binds CTLA4, an anti-PD 1 antibody or antigen-binding fragment thereof that specifically binds PD1, an anti-PD-L1 antibody or antigen-binding fragment thereof that specifically binds PD-L1, and a combination thereof.

116. The method of claim 115, wherein the checkpoint inhibitor polypeptide is an anti-PD-L1 antibody selected from atezolizumab, avelumab, or durvalumab.

117. The method of claim 115, wherein the checkpoint inhibitor polypeptide is an anti-CTLA-4 antibody selected from tremelimumab or ipilimumab.

118. The method of claim 115, wherein the checkpoint inhibitor polypeptide is an anti-PD1 antibody selected from nivolumab or pembrolizumab.

119. The method of any one of claims 111-118, wherein the immune checkpoint modulator is administered at a dosage level sufficient to deliver 100-300 mg to the subject.

120. The method of claim 119, wherein the immune checkpoint modulator is administered at a dosage level sufficient to deliver 200 mg to the subject.

121. The method of any one of claims 111-120, wherein the immune checkpoint modulator is administered by intravenous infusion.

122. The method of any one of claims 111-121, wherein the immune checkpoint modulator is administered to the subject twice, three times, four times or more.

123. The method of any one of claims 111-122, wherein the immune checkpoint modulator is administered to the subject on the same day as the mRNA vaccine administration.

124. The method of any one of claims 104-123, wherein the cancer is selected from:
(i) the group consisting of non-small cell lung cancer (NSCLC), small cell lung cancer, melanoma, bladder urothelial carcinoma, HPV-negative head and neck squamous cell carcinoma (HNSCC), and a solid malignancy that is microsatellite high (MSI H)/mismatch repair (MMR) deficient; and/or
(ii) cancer of the pancreas, peritoneum, large intestine, small intestine, biliary tract, lung, endometrium, ovary, genital tract, gastrointestinal tract, cervix, stomach, urinary tract, colon, rectum, and hematopoietic and lymphoid tissues.

125. The method of claim 124, wherein the NSCLC lacks an EGFR sensitizing mutation and/or an ALK translocation.

126. The method of claim 125, wherein the solid malignancy that is microsatellite high (MSI H)/mismatch repair (MMR) deficient is selected from the group consisting of colorectal cancer, stomach adenocarcinoma, esophageal adenocarcinoma, and endometrial cancer.

127. A method of producing an mRNA encoding a concatemeric cancer antigen comprising between 1000 and 3000 nucleotides, the method comprising:
(a) binding a first polynucleotide comprising an open reading frame encoding the cancer antigen of any one of claim 1-103 and a second polynucleotide comprising a 5′-UTR to a polynucleotide conjugated to a solid support;
(b) ligating the 3′-terminus of the second polynucleotide to the 5′-terminus of the first polynucleotide under suitable conditions, wherein the suitable conditions comprise a DNA Ligase, thereby producing a first ligation product;
(c) ligating the 5′ terminus of a third polynucleotide comprising a 3′-UTR to the 3′-terminus of the first ligation product under suitable conditions, wherein the suitable conditions comprise an RNA Ligase, thereby producing a second ligation product; and
(d) releasing the second ligation product from the solid support, thereby producing an mRNA encoding the concatemeric cancer antigen comprising between 1000 and 3000 nucleotides.

128. A method for treating a subject with a personalized mRNA cancer vaccine, comprising identifying a set of neoepitopes to produce a patient specific mutanome, selecting a set of neoepitopes for the vaccine from the mutanome based on MHC binding strength, MHC binding diversity, predicted degree of immunogenicity, low self reactivity, and/or T cell reactivity, preparing the mRNA vaccine to encode the set of neoepitopes, and administering the mRNA vaccine to the subject within two months of isolating the sample from the subject.

129. A method of identifying a set of neoepitopes for use in a personalized mRNA cancer vaccine having one or more polynucleotides that encode the set of neoepitopes comprising:
(a) identifying a patient specific mutanome by analyzing a patient transcriptome and a patient exome,
(b) selecting a subset of 15-500 neoepitopes from the mutanome using a weighted value for the neoepitopes based on at least three of: an assessment of gene or transcript-level expression in patient RNA-seq; variant call confidence score; RNA-seq allele-specific expression; conservative vs. non-conservative amino acid substitution; position of point mutation (Centering Score for increased TCR engagement); position of point mutation (Anchoring Score for differential HLA binding); Selfness: <100% core epitope homology with patient WES data; HLA-A and -B IC50 for 8 mers-11 mers; HLA-DRB1 IC50 for 15 mers-20 mers; promiscuity Score; HLA-C IC50 for 8 mers-1l mers; HLA-DRB3-5 IC50 for 15 mers-20 mers; HLA-DQB1/A1 IC50 for 15 mers-20 mers; HLA-DPB1/A1 IC50 for 15 mers-20 mers; Class I vs Class II proportion; Diversity of patient HLA-A, -B and DRB1 allotypes covered; proportion of point mutation vs complex epitopes; pseudo-epitope HLA binding scores; presence and/or abundance of RNAseq reads, and
(c) selecting the set of neoepitopes for use in a personalized mRNA cancer vaccine from the subset based on the highest weighted value, wherein the set of neoepitopes comprise 15-40 neoepitopes.

130. A method of identifying a set of neoepitopes for use in a personalized mRNA cancer vaccine having one or more polynucleotides that encode the set of neoepitopes comprising:
(a) generating a RNA-seq sample from a patient tumor to produce a set of RNA-seq reads,
(b) compiling overall counts of nucleotide sequences from all RNA-seq reads,
(c) comparing sequence information between the tumor sample and a corresponding database of normal tissues of the same tissue type, and
(d) selecting a set of neoepitopes for use in a personalized mRNA cancer vaccine from the subset based on the highest weighted value, wherein the set of neoepitopes comprise 15-40 neoepitopes.
